(19) United States
(12) Patent Application Publication
(10) Pub. No.: US 2023/0036181 A1
(43) Pub. Date: Feb. 2, 2023

(54) ANTI-GAL3 ANTIBODIES AND METHODS OF USE

(71) Applicant: TrueBinding, Inc., Foster City, CA (US)

(72) Inventors: Dongxu Sun, Los Altos, CA (US); Suhail Rasool, Palo Alto, CA (US); Catherine A. Gordon, Fremont, CA (US); Ke Hong, Saratoga, CA (US); Fan Chen, Sunnyvale, CA (US); Sara Matilda Bolin, Redwood City, CA (US); Ksenya Shchors, San Mateo, CA (US); Yadong Yu, Belmont, CA (US); Tsung-Huang Tsai, San Carlos, CA (US); Samuel A.F. Williams, Menlo Park, CA (US); Karan Lala, Palo Alto, CA (US); Heng Wu, Foster City, CA (US); Yan Wang, Concord, CA (US)

(21) Appl. No.: 17/812,159

(22) Filed: Jul. 12, 2022

Related U.S. Application Data
(63) Continuation of application No. PCT/US2021/013136, filed on Jan. 12, 2021.
(60) Provisional application No. 62/960,300, filed on Jan. 13, 2020.
(60) Provisional application No. 63/024,327, filed on May 13, 2020.
(60) Provisional application No. 63/092,069, filed on Oct. 15, 2020.
(60) Provisional application No. 63/122,409, filed on Dec. 7, 2020.

(51) Int. Cl. C07K 16/28; A61P 25/28

(52) U.S. Cl. CPC C07K 16/2851; A61P 25/28; C07K 2317/565; C07K 2317/76

(57) ABSTRACT

Disclosed herein are antibodies and compositions used for binding to Gal3. Some embodiments allow for disrupting interactions between Galectin-3 (Gal3) and cell surface markers and/or proteins associated with neurological diseases and/or proteopathies, such as Alzheimer's disease. Additionally, disclosed herein are methods of treatment and uses of the antibodies or binding fragments thereof for the treatment of fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, cancer, brain cancer, breast cancer, colorectal cancer, kidney cancer, liver cancer, lung cancer, pancreatic cancer, bladder cancer, stomach cancer, hematological malignancy, neurological diseases and/or proteopathies. Furthermore, some embodiments provided herein can cross the blood-brain barrier and can be conjugated or otherwise associated with one or more payloads for the treatment of a neurological disease.

### CROSS-REFERENCE TO RELATED APPLICATIONS

This application claims the benefit of priority of U.S. Provisional Patent Application No. 62/960,300, filed Jan. 13, 2020, U.S. Provisional Patent Application No. 63/024,327, filed May 13, 2020, U.S. Provisional Patent Application No. 63/092,069, filed Oct. 15, 2020, and U.S. Provisional Patent Application No. 63/122,409, filed Dec. 7, 2020, each of which is hereby expressly incorporated by reference in its entirety, including any appendices filed therewith.

### REFERENCE TO SEQUENCE LISTING

The present application is being filed along with a Sequence Listing in electronic format. The Sequence Listing is provided as a file entitled SeqListingIMMUT027WO.XML, which was created and last modified on Jun. 29, 2022, which is 1,230,757 bytes in size. The information in the electronic Sequence Listing is hereby incorporated by reference in its entirety.

### FIELD

Aspects of the present disclosure relate generally to antibodies or binding fragments thereof that bind to Galectin-3 (Gal3). These antibodies or binding fragments thereof can block or disrupt the interaction between Gal3 and cell surface markers and/or proteins associated with neurological disorders and/or proteopathies. These antibodies or binding fragments thereof can also cross the blood-brain barrier.

### BACKGROUND

Galectin-3 (Gal3, GAL3) is a lectin, or a carbohydrate-binding protein, with specificity towards beta-galactosides. In human cells, Gal3 is expressed and can be found in the nucleus, cytoplasm, cell surface, and in the extracellular space. Gal3 recognizes and interacts with beta-galactose conjugates on various proteins.

### SUMMARY OF THE DISCLOSURE

Disclosed herein are embodiments relating to anti-Gal3 antibodies, binding fragments thereof, and/or antigen binding molecules. In some embodiments, any such structures can be used to block an interaction between Gal3 and a cell surface marker.

In some embodiments, these cell surface markers are associated with a disease, for example, cancer or fibrosis. In some embodiments, any such structures prevent abnormal folding or accumulation of proteins. In some embodiments, any such structures can be used to treat a neurological disorder, such as but not limited to Alzheimer's disease.

In some embodiments, any such structures can be used to assist in crossing the blood brain barrier. In some embodiments, these items can be associated with one or more payload.

Disclosed herein are anti-Gal3 antibodies or binding fragments thereof comprising (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 36-44, 588-615. In some embodiments, the V_{H}-CDR2 comprises an amino acid sequence selected from SEQ ID NOs: 54-60, 616-643. In some embodiments, the V_{H}-CDR3 comprises an amino acid sequence selected from SEQ ID NOs: 70-81, 644-671. In some embodiments, the V_{L}-CDR1 comprises an amino acid sequence selected from SEQ ID NOs: 92-101, 672-699. In some embodiments, the V_{L}-CDR2 comprises an amino acid sequence selected from SEQ ID NOs: 111-116, 700-727. In some embodiments, the V_{L}-CDR3 comprises an amino acid sequence selected from SEQ ID NOs: 127-135, 728-755.

Also disclosed herein are methods of treating a neurological disorder in a subject in need thereof. In some embodiments, the methods comprise administering to the subject an effective amount of an anti-Gal3 antibody or binding fragment thereof, thereby treating the neurological disorder in the subject.

Also disclosed herein are methods of disrupting binding between Gal3 and APP or Aβ, or both. In some embodiments, the methods comprise contacting the APP or Aβ, or both, with an anti-Gal3 antibody or binding fragment thereof, thereby disrupting the binding between Gal3 and APP.

Also disclosed herein are methods of treating a proteopathy in a subject in need thereof. In some embodiments, the methods comprise administering to the subject an effective amount of an anti-Gal3 antibody or binding fragment thereof, thereby treating the proteopathy in the subject.

Also disclosed herein are methods of administering an antibody to a subject. In some embodiments, the methods comprise administering to the subject an anti-Gal3 antibody or binding fragment thereof.

Also disclosed herein are methods of promoting neuronal regeneration in a subject in need thereof. In some embodiments, the methods comprise administering to the subject an effective amount of an anti-Gal3 antibody or binding fragment thereof, thereby promoting neuronal regeneration in the subject.

Also disclosed herein are methods of disrupting binding between Gal3 and a cell surface receptor. In some embodiments, the methods comprise contacting Gal3 with an anti-Gal3 antibody or binding fragment thereof, thereby disrupting the binding between Gal3 and a cell surface receptor.

Also disclosed herein are methods of treating a disease such as an inflammatory disease, cancer, and/or fibrosis in a subject in need thereof. In some embodiments, the disease comprises fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, cancer, brain cancer, breast cancer, colorectal cancer, kidney cancer, liver cancer, lung cancer, pancreatic cancer, bladder cancer, stomach cancer, or a hematological malignancy. In some embodiments, the methods comprise administering to the subject an effective amount of an anti-Gal3 antibody or binding fragment thereof, thereby treating the disease in the subject.

Also disclosed here are anti-Gal3 antibodies or binding fragments thereof for the use in the treatment of a disease such as an inflammatory disease, cancer, and/or fibrosis in a subject in need thereof.

Also disclosed herein are anti-Gal3 antibodies or binding fragments thereof for use in the treatment of a neurodegenerative disorder in a subject in need thereof.

Also disclosed herein are anti-Gal3 antibodies or binding fragments thereof for use in the treatment of a proteopathy in a subject in need thereof.

Also disclosed herein are anti-Gal3 antibodies or binding fragments thereof for use in promoting neuronal regeneration in a subject in need thereof.

Also disclosed herein are antibody conjugates. In some embodiments, the antibody conjugates comprise an anti-Gal3 antibody or binding fragment thereof and a payload conjugated to the anti-Gal3 antibody or binding fragment thereof. In some embodiments, the antibody conjugate is able to cross the blood brain barrier. In some embodiments, the barrier is in a subject who has a blood brain barrier that is weakened or altered due to a disease that impacts the blood brain barrier, e.g., that decreases the structural integrity of the barrier.

Also disclosed herein are multi-specific antibodies. In some embodiments, the multi-specific antibodies comprise a first binding domain that binds to Gal3 and a second binding domain that binds to a therapeutic target molecule located in the brain of a subject.

Also disclosed herein are methods of delivering a payload to the central nervous system of a subject in need thereof. In some embodiments, the methods comprise administering to the subject an antibody conjugate comprising an anti-Gal3 antibody or binding fragment thereof and a payload conjugated to the anti-Gal3 antibody or binding fragment thereof, wherein the antibody conjugate is able to cross a blood-brain barrier. In some embodiments, the barrier is in a subject who has a blood brain barrier that is weakened or altered due to a disease that impacts the blood brain barrier, e.g., that decreases the structural integrity of the barrier.

Also disclosed herein are methods of increasing the permeability of a payload across the blood-brain barrier of a subject in need thereof. In some embodiments, the methods comprise conjugating an anti-Gal3 antibody or binding fragment thereof to the payload to form an antibody conjugate. In some embodiments, the barrier is in a subject who has a blood brain barrier that is weakened or altered due to a disease that impacts the blood brain barrier, e.g., that decreases the structural integrity of the barrier.

Also disclosed herein are uses of anti-Gal3 antibodies or binding fragments thereof to assist a payload to cross a blood brain barrier of a subject.

Also disclosed herein are methods of disrupting an interaction between Gal3 and a transforming growth factor beta (TGF-b) receptor.

Also disclosed herein are methods of treating fibrosis in a subject in need thereof.

Also disclosed herein are methods of treating non-alcoholic fatty liver disease (NAFLD) or non-alcoholic steatohepatitis (NASH) in a subject in need thereof.

Also disclosed herein are methods of treating an immune-related disorder in a subject in need thereof.

Also disclosed herein are methods of disrupting an interaction between Gal3 and a tumor cell surface marker.

Also disclosed herein are methods of treating cancer in a subject in need thereof.

Also disclosed herein are methods of identifying an antibody or binding fragment thereof as capable of disrupting an interaction between Gal3 and a TGF-b receptor, cell surface marker, or tumor cell surface marker.

Also disclosed herein are pharmaceutical compositions or medicaments. In some embodiments, the pharmaceutical compositions or medicaments comprise any one of the anti-Gal3 antibodies or binding fragments thereof, any one of the antibody conjugates, or any one of the multi-specific antibodies disclosed herein, and at least one pharmaceutically acceptable diluent, excipient, or carrier. In some embodiments, the composition or medicament is used for the treatment of fibrosis, liver fibrosis, non-alcoholic fatty liver disease (NAFLD), non-alcoholic steatohepatitis (NASH), kidney fibrosis, cardiac fibrosis, arterial fibrosis, venous thrombosis, or pulmonary fibrosis. In some embodiments, the composition or medicament is used for the treatment of cancer. In some embodiments, the composition or medicament is used for the treatment of an immune-related disorder.

Also disclosed herein are anti-Gal3 antibodies or binding fragments thereof for use in in the treatment of fibrosis, liver fibrosis, NAFLD, NASH, kidney fibrosis, cardiac fibrosis, arterial fibrosis, venous thrombosis, or pulmonary fibrosis.

Also disclosed herein are anti-Gal3 antibodies or binding fragments thereof for use in the treatment of cancer.

Also disclosed herein are anti-Gal3 antibodies or binding fragments thereof for use in the inhibition of tumor cell growth in vitro.

Also disclosed herein are anti-Gal3 antibodies or binding fragments thereof for use in the retardation of brain tumor growth.

Also disclosed herein are antibodies that bind to human Gal3 and competes with an anti-Gal3 antibody or binding fragment thereof for binding to human Gal3. In some embodiments, the antibodies compete with any one of the anti-Gal3 antibodies or binding fragments disclosed herein.

Also disclosed herein are methods for identifying an antibody or binding fragment capable of disrupting an interaction between Gal3 and a TGF-b receptor.

Also disclosed herein are antibodies or binding fragments thereof that bind to an N-terminal domain and/or TRD of Gal3.

Also disclosed herein are proteins comprising one or more peptide sequences having at least 80%, 85%, 90%, 95%, 99%, or 100% homology to one or more peptide sequences of FIG. 18-27.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIG. 1 depicts a graphical representation of the assessment of relative binding affinity of APP695 to Galectin-3 (GAL3) from different sources as measured by enzyme-linked immunosorbent assay (ELISA).

FIG. 2 depicts a graphical representation of the assessment of relative binding affinity of APP695 and GAL3 following blockade by anti-GAL3 antibodies as measured by ELISA.

FIG. 3 depicts the results for hippocampal dependent memory test (Morris water maze) for APPSwe transgenic mice treated with isotype control or anti-GAL3 antibody (TB001) and wild type control mice, before and after antibody treatment.

FIG. 4 depicts a graphical representation of the number of crosses during the probe trail phase of the Morris water maze for APPSwe transgenic animals and wild type control.

FIG. 5A depicts the results of analysis of Aβ protein levels in brain tissue of APPSwe transgenic and wild type mice determined by immune-blotting using monoclonal Aβ specific sequence dependent antibody (6E10).

FIG. 5B depicts a graphical representation of the intensity of the bands of FIG. 5A determined by Image J software.

FIG. 5C depicts the results of analysis of mTB001 in brain tissue of APPSwe transgenic and wild type mice as measured by ELISA.

FIG. 6A-B show the results for the Morris water maze test for Aβ42 fibril-injected mice treated with isotype control or anti-GAL3 antibody (TB001) and wild type (not injected) control, before (6A) and after (6B) antibody treatment.

FIG. 7 depicts a graphical representation of the number of crosses during the probe trial phase of the Morris water maze test for Aβ42 fibril-injected mice treated with isotype control or anti-GAL3 antibody (TB001) and wild type (not injected) control.

FIG. 8A depicts a graphical representation of the results of immunohistochemical staining of the levels of Aβ with 6E10 antibody in mouse brain tissue quantified by NIH Image J software.

FIG. 8B depicts a graphical representation of the results of immunohistochemical staining of the levels of NeuN in mouse brain tissue quantified by NIH Image J software.

FIG. 8C depicts a graphical representation of the results of immunohistochemical staining of the levels of Phospho-Tau in mouse brain tissue quantified by NIH Image J software.

FIG. 8D depicts a graphical representation of the results of immunohistochemical staining of the levels of Iba-1 in mouse brain tissue quantified by NIH Image J software.

FIG. 8E depicts a graphical representation of the results of immunohistochemical staining of the levels of Galectin-3 in mouse brain tissue quantified by NIH Image J software.

FIG. 9 depicts a graphical representation of the immunoblot bands intensity of A3 protein levels in brain tissue of Aβ42 fibril-injected and wild type mice analyzed by Image J software.

FIG. 10A-B depict graphical representations of the assessment of relative binding affinity of Aβ42 peptide (10A) or Aβ42 oligomer (10B) to Gal3 from different sources as measured by ELISA.

FIG. 11A-B depicts graphical representations of the assessment of relative binding affinity of Aβ42 peptide (11A) or Aβ42 oligomer (11B) following blockade by anti-Gal3 antibodies as measured by ELISA.

FIG. 11C depicts a graphical representation of the comparison of the efficacy of blocking the interaction by Aβ42 and Gal3 between anti-Gal3 antibodies (TB001 and TB006) or the small molecule Gal3 inhibitor TD139.

FIG. 12A depicts a graphical representation of the assessment of relative binding affinity of TLR4 to Gal3 from different sources as measured by ELISA.

FIG. 12B depicts a graphical representation of the assessment of relative binding affinity of TLR4 and Gal3 following blockade by anti-Gal3 antibodies as measured by ELISA.

FIG. 13A depicts a graphical representation of the assessment of relative binding affinity of TREM2 to Gal3 from different sources as measured by ELISA.

FIG. 13B depicts a graphical representation of the assessment of relative binding affinity of TREM2 and Gal3 following blockage by anti-Gal3 antibodies as measured by ELISA.

FIG. 14A depicts a graphical representation of the assessment of relative binding affinity of Tau oligomers and Gal3 following blockade by anti-Gal3 antibodies as measured by ELISA.

FIG. 14B depicts a graphical representation of the assessment of relative binding affinity of Tau oligomers to Gal3 from different sources as measured by ELISA.

FIG. 15A depicts a graphical representation of the assessment of relative binding affinity of alpha-synuclein and Gal3 following blockade by anti-Gal3 antibodies as measured by ELISA.

FIG. 15B depicts a graphical representation of the assessment of relative binding affinity of alpha-synuclein to Gal3 from different sources as measured by ELISA.

FIG. 16 depicts protein sequences of Gal3, amyloid-beta precursor protein (APP) isoform c (APP695), amyloid-beta peptide (1-42), TGF-b receptors, and other designated protein sequences.

FIG. 17 depicts peptide sequences of Gal3 used to generate and analyze antibodies.

FIG. 18 depicts exemplary variable heavy chain complementarity-determining region (CDR) 1 for anti-Gal3 antibodies disclosed herein. In some embodiments, any of the compositions or methods provided herein can include one or more of the CDRs provided herein.

FIG. 19 depicts exemplary variable heavy chain CDR2 for anti-Gal3 antibodies disclosed herein. In some embodiments, any of the compositions or methods provided herein can include one or more of the CDRs provided herein.

FIG. 20 depicts exemplary variable heavy chain CDR3 for anti-Gal3 antibodies disclosed herein. In some embodiments, any of the compositions or methods provided herein can include one or more of the CDRs provided herein.

FIG. 21 depicts exemplary variable light chain CDR1 for anti-Gal3 antibodies disclosed herein. In some embodiments, any of the compositions or methods provided herein can include one or more of the CDRs provided herein.

FIG. 22 depicts exemplary variable light chain CDR2 for anti-Gal3 antibodies disclosed herein. In some embodiments, any of the compositions or methods provided herein can include one or more of the CDRs provided herein.

FIG. 23 depicts exemplary variable light chain CDR3 for anti-Gal3 antibodies disclosed herein. In some embodiments, any of the compositions or methods provided herein can include one or more of the CDRs provided herein.

FIG. 24 depicts exemplary heavy chain variable region sequences for anti-Gal3 antibodies disclosed herein. In some embodiments, any of the compositions or methods provided herein can include one or more of the VH sequences provided herein.

FIG. 25 depicts exemplary light chain variable region sequences for anti-Gal3 antibodies disclosed herein. In some embodiments, any of the compositions or methods provided herein can include one or more of the VL sequences provided herein.

FIG. 26 depicts exemplary heavy chain sequences for anti-Gal3 antibodies disclosed herein. In some embodiments, any of the compositions or methods provided herein can include one or more of the heavy chain sequences provided herein.

FIG. 27 depicts exemplary light chain sequences for anti-Gal3 antibodies disclosed herein. In some embodiments, any of the compositions or methods provided herein can include one or more of the light chain sequences provided herein.

FIG. 28 depicts exemplary combinations of variable heavy chain CDR1, CDR2, and CDR3 of anti-Gal3 antibodies disclosed herein. In some embodiments, any of the compositions or methods provided herein can include one or more of the heavy chain CDR combinations provided herein.

FIG. 29 depicts exemplary combinations of variable light chain CDR1, CDR2, and CDR3 of anti-Gal3 antibodies disclosed herein. In some embodiments, any of the compositions or methods provided herein can include one or more of the light chain CDR combinations provided herein.

FIG. 30 depicts exemplary combinations of heavy and light chain CDRs of anti-Gal3 antibodies disclosed herein. In some embodiments, any of the compositions or methods provided herein can include one or more of the heavy and light chain CDR combinations provided herein.

FIG. 31 depicts exemplary combinations of heavy and light chain variable regions of anti-Gal3 antibodies disclosed herein. In some embodiments, any of the compositions or methods provided herein can include one or more of the heavy and light chain variable region combinations provided herein.

FIG. 32 depicts exemplary combinations of heavy and light chains of anti-Gal3 antibodies disclosed herein. In some embodiments, any of the compositions or methods provided herein can include one or more of the heavy and light chains provided herein.

FIG. 33 depicts the peptides that were found to bind to exemplary antibodies disclosed herein (according to the peptide nomenclature depicted in FIG. 17 and discussed herein) and binning of these exemplary antibodies.

FIG. 34A depicts plasma concentrations of exemplary anti-Gal3 antibodies conjugated to biotin in C57BL6 mice transplanted with GL261-LUC murine glioblastoma tumors at four days following i.v. administration of the anti-Gal3 antibodies.

FIG. 34B depicts concentrations of exemplary anti-Gal3 antibodies conjugated to biotin found in tumors and normal brain tissue of C57BL6 mice transplanted with GL261-LUC murine glioblastoma tumors at four days following i.v. administration of the anti-Gal3 antibodies.

FIG. 34C depicts the relative concentration of anti-Gal3 antibodies conjugated to biotin found in either tumors or normal brain tissue of C57BL6 mice transplanted with GL261-LUC murine glioblastoma tumors compared to their respective plasma at four days following i.v. administration of the anti-Gal3 antibodies.

FIG. 34D depicts an immunoblot of the apoptosis marker PARP and GAPDH loading control in brain tumor lysates isolated from C57BL6 mice transplanted with GL261-LUC murine glioblastoma tumors following i.v. administration of anti-Gal3 antibodies.

FIG. 34E depicts a graphical representation of relative amounts of PARP normalized to GAPDH loading control quantified from the immunoblot of FIG. 34D.

FIG. 35 depicts alignments of some embodiments of the VH CDR or VL CDR regions of various embodiments of anti-Gal3 antibodies. In some embodiments, any of the methods or compositions provided herein can use any 1, 2, 3, 4, 5, 6, or 7 of the consensus CDRs provided herein.

FIG. 36 depicts KD (M) values of Gal3 binding for exemplary anti-Gal3 antibodies disclosed herein.

FIG. 37 depicts nucleic acid sequences that encode for exemplary heavy chain variable regions of anti-Gal3 antibodies disclosed herein. In some embodiments, any of the compositions or methods provided herein can include one or more of the heavy chain variable regions encoded by the nucleic acids provided herein.

FIG. 38 depicts nucleic acid sequences that encode for exemplary light chain variable regions of anti-Gal3 antibodies disclosed herein. In some embodiments, any of the compositions or methods provided herein can include one or more of the light chain variable regions encoded by the nucleic acids provided herein.

FIG. 39 depicts nucleic acid sequences that encode for exemplary heavy chains of anti-Gal3 antibodies disclosed herein. In some embodiments, any of the compositions or methods provided herein can include one or more of the heavy chains encoded by the nucleic acids provided herein.

FIG. 40 depicts nucleic acid sequences that encode for exemplary light chains of anti-Gal3 antibodies disclosed herein. In some embodiments, any of the compositions or methods provided herein can include one or more of the light chains encoded by the nucleic acids provided herein.

FIG. 41A depicts a graphical representation of the assessment of relative binding affinity of transforming growth factor beta (TGF-b) receptor type 1 (TGFBR1), TGF-b receptor type 2 (TGFBR2), TGF-b receptor type 3 (TGFBR3) or combinations thereof to Galectin-3 (GAL3) as measured by enzyme-linked immunosorbent assay (ELISA).

FIG. 41B depicts binding kinetics of the interaction between Gal3 and TGF-b receptors as measured by surface plasmon resonance.

FIG. 42 depicts a graphical representation of the assessment of relative binding affinity of TGFBR1 and GAL3 following blockade by anti-GAL3 antibodies as measured by ELISA.

FIG. 43A depicts a graphical representation of the assessment of relative expression of genes associated with fibrosis in LX2 cells treated with TGF-b and either murine anti-GAL3 antibodies or vehicle control as measured by quantitative reverse transcriptase polymerase chain reaction (qRT-PCR).

FIG. 43B depicts a graphical representation of the assessment of relative expression of genes associated with fibrosis in LX2 cells treated with TGF-b and either humanized anti-GAL3 antibodies or vehicle control as measured by qRT-PCR.

FIG. 44A-D depicts graphical representations of the assessment of relative binding affinity of (A) VEGFR1, VEGFR2, VEGFR3, EGFR, PDGFRa, PDGFRb; (B) ErbB2, HGFR (cMet), TNF sRI, CTLA4, CD47, PD-L1; (C) FGFR1 alpha-IIIb; (D) FGFR1 alpha-IIIc, FGFR2 alpha-IIIc, FGFR IIIc, FGFR4 to Galectin-3 as measured by enzyme-linked immunosorbent assay (ELISA).

FIG. 44E depicts a graphical representation of the determination of binding affinity of VEGFR2, VEGFR3, EGFR, PDGFRa, PDGFRb and ErbB2 to Gal3 as measured by SPR.

FIG. 45A-E depicts graphical representations of the assessment of relative binding affinity of tumor surface receptors (A) EGFR, (B) VEGFR2, (C) VEGFR3, (D) PDGFRa, (E) PDGFRb to Gal3 following blockade by anti-Gal3 antibodies as measured by ELISA.

FIG. 46A-B depict graphical representations of the determination of binding affinity of anti-Gal3 antibody (A) clone 6H6 and (B) clone 2D10 to full length recombinant human Gal3 (rhGal3) or the C-terminal domain of Gal3 (Gal3-CRD) as measured by SPR.

FIG. 47A-B depict graphical representations of the percent of survival for (A) hepatocellular carcinoma (HCC) cells (Hep3B, HepG2) and (B) glioblastoma (GBM) tumor cells (U118) following exposure to anti-Gal3 antibodies for the duration of 72 hours compared to untreated control.

FIG. 48 depicts a graphical representation of the percent of survival for GBM tumor cells (lines U87MG, U118, LN229) following exposure to control isotype and anti-Gal3 antibodies (2D10) alone, or in combination with 100 μM of temozolomide (TMZ) for the duration of 72 hours compared to untreated control.

FIG. 49 depicts a graphical representation of tumor progression in GL261-LUC transplanted animals treated with control isotype, TMZ, anti-Gal3 antibody (2D10), or combination (TMZ+2D10) determined as a fold change of luminescent emission (flux per second) following initiation of the treatment.

FIG. 50 depicts antibody affinities (K_{D}) of anti-Gal3 humanized antibodies IMT001 (TB001) and IMT006 (TB006; 4A11.H3L1) for human, cynomolgus, and mouse Gal3. Humanized IMT001 and IMT006, derived from mouse mAbs, both have high affinity for human and cynomolgus Gal3, whereas IMT001 also has high affinity for mouse Gal3.

FIG. 51 depicts a graphical representation of the inhibition of TGF-β-induced pro-collagen production in LX-2 cells when treated with IMT001 (TB001), IMT006 (TB006; 4A11.H3L1), and hIgG4 (isotype control) at increasing concentrations of antibodies. LX-2 cells were stimulated with TGF-b (10 ng/mL) for 2 hours.

FIG. 52 depicts a graphical representation of the inhibition of TGF-β-induced pro-collagen production and Gal3 expression in LX-2 cells when treated with IMT001 and 4A11.H3L1. TGF-b stimulated expression of pro-collagen in LX-2 cells is augmented by exogenous Gal3 and inhibited by IMT006. Gal3 increased on the LX-2 cell surface (panel A) and in the culture medium (panel B) in response to TGF-b stimulation. Anti-Gal3 antibody IMT006 reduced pro-collagen protein after either TGF-b treatment or TGF-b plus rhGal3.

FIG. 53 depicts a graphical representation of the reduction of Gal3 and membrane TGFBR2 expression in LX2 cells transfected with a Gal3 short hairpin RNA (shRNA) vector, and reduction of membrane TGFbR1 expression in control LX-2 cells treated with IMT001. TGFb-R2 and Gal3 expression reduced on cell surface of LX-2 cells as a result of knockdown of Gal3. LX-2 cells were transfected with either a short hairpin RNA vector to silence Gal3 or a control vector and single clones were isolated (named LX-shGal3 and LX2-shCon, respectively). The expression of Gal3 is markedly reduced in the LX2-shGal3 in comparison to LX2-shCon, following treatment with TGF-b. The expression of TGFbR2 and Gal3 on cell membranes of LX2-shGal3 cells was reduced compared to LX2-shCon cells. On non-transfected LX-2 cells, treatment with IMT001 reduced cell membrane TGFbR1.

FIG. 54 depicts a graphical representation of the inhibition of TGF-β-induced pro-collagen production in LX-2 cells transfected with a Gal3 shRNA vector. Knockdown of Gal3 in LX-2 cells reduces TGF-b induced pro-collagen. TGF-b EC₅₀ for pro-collagen production in LX2-scramble control was 1.01 ng/mL, and TGF-b EC₅₀ for pro-collagen production in LX2-shGal3 Gal3-knockdown cells was 2.04 ng/mL.

FIG. 55 depicts a graphical representation of the pharmacokinetics of IMT001 in rat, where the half-life is approximately 2 weeks. PK of IMT001 is dose proportional in rat with half-life of approximately 2 weeks.

FIG. 56 depicts a graphical representation of the tissue distribution of TB006 (IMT006, 4A11.H3L1) in mice after a single injected dose. ELISA was used to measure IMT006 exposure in plasma and tissues.

FIG. 57 depicts a graphical representation of assaying total and unbound Gal3 in rat plasma after treatment with IMT001. A single dose of IMT001 was given at 3 mg/kg (n=3) and 30 mg/kg (n=4) via i.v. Samples were taken 21 days after dosing. In SD rats treated with 30 mg/kg IMT001, there was a 2.97 fold increase in total rGal3. Unbound rGal3 was ˜85% lower than total rGal3 in SD rats treated with 30 mg/kg IMT001. In comparison to untreated rats, unbound rGal3 was 55% lower after treatment with IMT001.

FIG. 58 depicts a graphical representation of the transcriptomics in methionine-choline deficient (MCD) mice treated with mIMT001 (murine IMT001). Statistical analysis was performed by Student's T-test; * p<0.05, ** p<0.01, *** p<0.001. Significantly enriched biological functions (by clusterProfiler analysis) were observed for the genes whose expression is both induced in the NASH model and inhibited by Ab treatment (the overlap in the Venn diagram).

FIG. 59 depicts a graphical representation of Gal3 and TGFbR1 expression in the liver of MCD mice after treatment with mIMT001.

FIG. 60 depicts a graphical representation of Gal3 and TGFbR1 expression in the liver of MCD mice after treatment with mIMT001.

FIG. 61 depicts a graphical representation of a co-immunoprecipitation analysis of TGFbR1 and TGFbR2 binding to immunoprecipitated Gal3. 293T cells were transfected with TGFbR1, TGFbR2, and Gal3-FLAG plasmids alone or in combination as depicted. The cell lysate were collected 24 hours after transfection and analyzed by FLAG IP. Over-expressed Gal3 pulled down TGFbR1/2 with high specificity. The upper bands in TGFbR2 blot are glycosylated TGFbR2 and the lower ones are non-glycosylated TGFbR2.

FIG. 62 depicts a graphical representation of Western blot analysis of Smad3 expression and phosphorylation status in LX-2 cells following TGF-β stimulation. LX2 cells were starved for 24 hours. The cells were exposed to 2 ng/mL of TGF-β alone or in conjunction with control antibody, IMT001, or IMT006 at the indicated concentrations. Cell lysates were analyzed for the levels of SMAD3 and phosphorylated SMAD3 proteins. GAPDH levels were used as a loading control.

FIG. 63A depicts a Western blot showing that Gal3 promotes aggregation of Aβ into oligomeric forms. Aβ oligomers were detected with antibody A11, and total Aβ was detected with antibody 6E10.

FIG. 63B depicts a dot blot of Aβ oligomer incubated with different concentrations of the anti-Gal3 antibody mTB001 (0, 10, 100 μg). The dot blots show that Aβ oligomerization was reversed by the anti-Gal3 antibody. Aβ oligomers were detected with antibody A11, and total Aβ was detected with antibody 6E10.

FIG. 63C depicts the quantification of the dot blot of FIG. 63B detecting with the Aβ oligomer antibody A11.

FIG. 63D depicts a dot blot of Aβ oligomer incubated with different anti-Gal3 antibodies disclosed herein. The number labels correspond to an anti-Gal3 antibody as depicted in this figure.

FIG. 64 depicts antibody names used throughout the present disclosure refer to the same antibody (with exemplary peptide and nucleic acid sequences provided elsewhere in the disclosure and appropriately attributed to at least one of the depicted names) and may be used interchangeably. The names shown in a column correspond to the same antibody.

FIG. 65A depicts a dot blot of a time-course of the aggregation of Aβ-42 peptide into oligomeric form when incubated with various isoforms of Gal3. The isoforms of Gal3 tested include full length Gal3 (denoted as E. coli), hGal3-R186S, hGal3-P64H, hGal3-65-250 (amino acids 65-250), and hGal3-CRD-His (a His-tagged C-terminal domain of Gal3). The “0” lane denotes no Gal3 added. The time-course was performed over 5 hours.

FIG. 65B depicts the quantification of the dot blot of FIG. 65A.

FIG. 65C depicts a dot blot of a time-course of the aggregation of Aβ-42 peptide into oligomeric form when incubated with various short peptides of Gal3. Peptides A-F were tested (SEQ ID NO: 582-587). hGal3-65-250 was used as a positive control.

### DETAILED DESCRIPTION OF THE DISCLOSURE

Some embodiments provided herein relate to anti-Gal3 antibodies or binding fragments thereof. In some embodiments, the anti-Gal3 antibodies or binding fragments thereof bind to the N-terminal domain of Gal3, the N-terminus of Gal3, or the tandem repeat domain (TRD) of Gal3.

Some embodiments provided herein relate to anti-Gal3 antibodies or binding fragments thereof that disrupt the interaction between Gal3 and a protein associated with proteopathies or neurological disease. In some embodiments are methods and uses of the anti-Gal3 antibodies and binding fragments thereof disclosed herein for the treatment of proteopathies and/or neurological disease.

Some embodiments provided herein relate to anti-Gal3 antibodies or binding fragments thereof that are able to cross the blood-brain barrier. In some embodiments, the blood-brain barrier is of a subject that has a neurological disease. In some embodiments, the anti-Gal3 antibodies or binding fragments thereof are multi-specific antibodies in order to increase the permeability of another antibody across the blood-brain barrier. In some embodiments, the anti-Gal3 antibodies or binding fragments thereof are conjugated to a payload in order to increase the permeability of the payload across the blood-brain barrier.

Some embodiments provided herein relate to anti-Gal3 antibodies or binding fragments thereof that disrupt the interaction between Gal3 and a cell surface marker or a tumor cell surface marker. In some embodiments are methods and uses of the anti-Gal3 antibodies and binding fragments thereof disclosed herein for the treatment of diseases associated with the cell surface marker or tumor cell surface marker. In some embodiments, the disease is a cancer, fibrosis, or immune-related disorder.

Galectin-3 (Gal3, GAL3) plays an important role in cell proliferation, adhesion, differentiation, angiogenesis, and apoptosis. This activity is, at least in part, due to immunomodulatory properties and binding affinity towards other immune regulatory proteins, signaling proteins, and other cell surface markers. Gal3 functions by distinct N-terminal and C-terminal domains. The N-terminal domain (isoform 1: amino acids 1-111) comprise a tandem repeat domain (TRD, isoform 1: amino acids 36-109) and is largely responsible for oligomerization of Gal3. The C-terminal domain (isoform 1: amino acids 112-250) comprise a carbohydrate-recognition-binding domain (CRD), which binds to β-galactosides.

Galectin-3 (Gal3) has been implicated to have immunomodulatory activity. An example of this is the interaction between Gal3 and T-cell immunoglobulin and mucin-domain containing-3 (TIM-3), which causes suppression of immune responses such as T cell activation and may enable cancer cells to evade immune clearance. This phenomenon and methods to inhibit the same are explored in WO 2019/023247, hereby expressly incorporated by reference in its entirety. Anti-Gal3 antibodies and methods of use thereof have also been explored, for example, in PCT Publication WO 2020/160156, hereby expressly incorporated by reference in its entirety.

There is a lasting need for a deeper understanding of whether Gal3 plays a role in diseases. Some diseases that may be associated with Gal3 include cancer, fibrosis, inflammatory diseases, neurological diseases and proteopathies such as Alzheimer's disease. There is also a need for the development of new and improved treatments for these diseases.

Disclosed herein are various embodiments of anti-Gal3 antibodies or binding fragments thereof and methods of use, for example, for the treatment of the diseases provided above or otherwise herein.

Disclosed herein are antibodies or binding fragments thereof and compositions thereof that bind or are selective towards Gal3. Also disclosed herein are methods for disrupting interactions between Gal3 and cell surface markers and/or proteins associated with fibrosis, liver fibrosis, kidney fibrosis, cardiac fibrosis, pulmonary fibrosis, non-alcoholic fatty liver disease, non-alcoholic steatohepatitis, sepsis, atopic dermatitis, psoriasis, cancer, brain cancer, breast cancer, colorectal cancer, kidney cancer, liver cancer, lung cancer, pancreatic cancer, bladder cancer, stomach cancer, hematological malignancy, neurodegenerative diseases and/or proteopathies (e.g. those caused by the misfolding or aggregation of proteins in a subject) with anti-Gal3 antibodies or binding fragments thereof, such as to treat a disease in a subject.

In some embodiments, the methods involve an antibody that binds to Gal3 and disrupts an interaction between Gal3 and another protein. This can be a direct obstruction of the interaction zone between Gal3 and the other protein, or an indirect alteration, such as a binding that results in a conformational change of Gal3, so that it no longer binds or is active with the other protein. It can also result by binding to a first section of Gal3, where some other part of the antibody obstructs or alters the interaction between Gal3 and the other protein. In some embodiments, the first section of Gal3 is the N-terminal domain of Gal3, the tandem repeat domain (TRD) of Gal3, or the C-terminal domain of Gal3. In some embodiments, the antibody that binds to Gal3 does not bind to the C-terminal domain of Gal3. In some embodiments, any of the anti-Gal3 antibodies or binding fragments thereof or any arrangement of any of the anti-Gal3 antibodies or binding fragments provided herein may be substituted with an antigen binding molecule that binds to Gal3.

Alzheimer's disease (Aβ) is a progressive neurodegenerative disorder and the most common type of dementia. Amyloid beta (Aβ) is a major constituent of amyloid plaques and so is suspected to be a pathogenic contributor to AD. The proteolytic cleavage of amyloid precursor protein (APP, including isoforms such as APP695) generates Aβ peptide, the aggregation of which is associated with the development of Alzheimer's disease. Serum level of Gal3 increases with the severity of memory loss in AD patients. Gal3 is specifically expressed in microglia associated with Aβ plaques. Gal3 expression is also significantly increased in the frontal lobe of AD patients in parallel with enhanced Aβ oligomerization. O-glycosylation can take place at Tyr-10 of the Aβ peptide in human cerebrospinal fluid and is increased in AD patients.

Disclosed herein are antibodies and binding fragments thereof that are specific for Gal3, and methods of use thereof for the treatment or prophylaxis of a neurodegenerative disease and/or proteopathy (e.g. Alzheimer's disease). The anti-Gal3 antibodies and binding fragments thereof disclosed herein disrupt the interaction between Gal3 and proteins associated with neurodegenerative diseases and/or proteopathies. In some embodiments, the proteins associated with neurodegenerative diseases and/or proteopathies cause disease due to misfolding or aggregation of the proteins in a subject. One non-limiting example of proteins associated with neurodegenerative diseases and/or proteopathies is amyloid-beta (Aβ) peptide.

In some embodiments, the anti-Gal3 antibodies or binding fragments disclosed herein disrupt the interaction between Gal3 and APP695. Some exemplary antibodies that strongly disrupt (e.g. at least 90%) the interaction between Gal3 and APP695 include but are not limited to 19B5.2E6, 7D8.2D8, F846C.1B2, F846C.1H12, F846TC.14A2, F849C.8D10, F849C.8H3, 4A11.H3L1 [IMT006-5 (TB006)], 15F10.2D6, F846TC.16B5, 23H9.2E4, F846C.1F5, IMT001-4 [TB001], F846C.2H3, 14H10.2C9, 15FG7.2A7, 20H5.A3, F846TC.14E4, 3B11.2G2, 20D11.2C6, and 2D10.2B2. Some exemplary antibodies that moderately disrupt (e.g. at least 45%) the interaction between Gal3 and APP695 include but are not limited to 13G4.2F8, F846TC.7F10, F847C.12F12, and F847C.4B10. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibodies or binding fragments disclosed herein disrupt the interaction between Gal3 and Aβ (e.g. Aβ monomer, oligomer, or fibril, or any combination thereof). Some exemplary antibodies that strongly disrupt (e.g. at least 90%) the interaction between Gal3 and Aβ monomer include but are not limited to 2D10.2B2, 20D11.2C6, 3B11.2G2, 20H5.A3, 846TC.14E4, 15G7.2A7, 14H10.2C9, 846C.2H3, TB001, 846C.1F5, 846TC.16B5, TB006, 846C.1B2, 846TC.14A2, 849C.8D10, and 19B5.2E6. Some exemplary antibodies that strongly disrupt (e.g. at least 90%) the interaction between Gal3 and Aβ oligomer include but are not limited to 2D10.2B2, 20D11.2C6, 3B11.2G2, 20H5.A3, 846TC.14E4, 14H10.2C9, TB001, 846C.1F5, and TB006. In some embodiments, the anti-Gal3 antibodies or binding fragments disclosed herein block the interaction between Gal3 and Aβ oligomer better than the small molecule Gal3 inhibitor TD139. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, administering any one of the anti-Gal3 antibodies or binding fragments disclosed herein can do at least one of, if not both of, enhancing the cognitive function and/or attenuate the accumulation of toxic conformational species of Aβ such as Aβ oligomers and/or Aβ fibrils in a subject.

In some embodiments, administering any one of the anti-Gal3 antibodies or binding fragments disclosed herein can reduce inflammation (e.g. of the brain) and/or encephalitis in a subject.

In some embodiments, administering any one of the anti-Gal3 antibodies or binding fragments disclosed herein can do one or more of reducing phospho-Tau levels, reducing activation of microglia (as detected by Iba-1 antibody), or reducing Gal3 levels in the brain of a subject.

In some embodiments, administering any one of the anti-Gal3 antibodies or binding fragments disclosed herein can do at least one of, if not both of, regenerate neuronal structures and/or reduce extracellular Aβ in a subject.

In some embodiments, administering any one of the anti-Gal3 antibodies or binding fragments disclosed herein can promote the phagocytic function of microglia and promote clearance of Aβ deposits in a subject.

In some embodiments, administering any one of the anti-Gal3 antibodies or binding fragments disclosed herein can inhibit Aβ aggregates (e.g. Aβ oligomer or Aβ fibril)-mediated activation of microglia in a subject.

In some embodiments, administering any one of the anti-Gal3 antibodies or binding fragments disclosed herein can block the interaction between Gal3 and TLR4 or TREM2, or both.

Also provided herein are embodiments that relate to anti-Gal3 antibodies or binding fragments and their use in methods to disrupt the interaction between Gal3 and cell surface markers such as TGF-β, VEGFR1, VEGFR2, VEGFR3, EGFR, PDGFRa, PDGFRb, ErbB2, HGFR, TNF sRI, CTLA4, CD47, PD-L1, FGFR, FGFR1 alpha-IIIb, FGFR1 alpha-IIIc, FGFR IIIc, or FGFR4. In some embodiments, this disruption can be used to alter biological processes that these cell surface markers regulate. In some embodiments, the cell surface markers are tumor cell surface markers, cancer cell surface markers, or fibrotic cell surface markers.

Cells use a wide range of signaling molecules and cognate cell surface receptors for signal transduction and cell communication. Abnormal functions of these interactions have been implicated in many diseases and disorders. For example, TGF-β is a potent signaling molecule acting with pleiotropic effects, including modulation of immune processes during the progression of cancer or fibrosis such as liver fibrosis.

As an example, the biological processes regulated by TGF-P include (but are not necessarily limited to): a) TGF-β regulates many biological responses including tissue fibrosis (liver, kidney, lung, heart, etc.), cell proliferation, apoptosis, differentiation, autophagy and the immune response; b) TGF-β has essential roles in the liver physiology and pathology and contributes to all stages of disease progression: from liver injury through inflammation, fibrosis, cirrhosis and hepatocellular carcinoma; c) TGF-β also mediates an epithelial-mesenchymal transition process in hepatocytes that may contribute, directly or indirectly, to increase the myofibroblast (MFB) population; hepatic stellate cell (HSC) activation is one of the most important steps during liver fibrosis; d) TGF-β plays an essential role in the activation of HSC to MFB (MFBs are the principal source of extracellular matrix protein accumulation and prominent mediators of fibrogenesis). Thus, in some embodiments, any one or more of the above processes can be disrupted by the use of a Gal3 antibody that reduces the binding between Gal3 and TGF-β receptors.

TGF-β binds to the TGF-3RII receptor, which binds and phosphorylates TGF-βRI, triggering recruitment of the receptor-regulated SMAD protein (R-SMAD) SMAD2 and SMAD3 to the cytoplasmic domain of activated TGF-βRI, which then phosphorylates SMAD2/3. Once phosphorylated, SMAD2/3 forms a trimer with SMAD4, which then translocates to the nucleus where it binds to SMAD-binding elements to modulate gene expression. In some embodiments, the antibodies or binding fragments provided herein alter TGF-β bindings to TGF-βRII, via altering how TGF-β binds to its receptors. In some embodiments, the antibodies or binding fragments do not alter TGF-β binding to TGF-βRII, via altering how TGF-β binds to Gal3.

TGF-β activates numerous SMAD-independent signaling pathways, called non-canonical TGF-β pathways such as WNT, ERK, P38, MAPK, PI3K, and AKT pathways. In some embodiments, the antibodies or binding fragments provided herein can alter how TGF-β activates these numerous SMAD-independent signaling pathways by altering how TGF-β binds to its receptors.

Inflammation plays a key role in liver fibrosis development. After injury occurs, infiltrating immune cells (macrophages, lymphocytes, eosinophils, and plasma cells) are recruited to the damaged site. Lymphocytes produce secreted protein signaling molecules termed cytokines and chemokines that activate macrophages. Activated macrophages, in turn, stimulate inflammatory cells such as lymphocytes, among others, contributing to the sustained maintenance of a pro-inflammatory environment. During fibrosis, macrophages produce profibrotic factors such as TGF-β and platelet derived growth factor (PDGF), control extracellular matrix turnover by regulating the balance of various matrix metalloproteases (MMPs) and tissue inhibitors of metalloproteinases (TIMPs), and are found very close to collagen-producing myofibroblasts, suggesting that the macrophages are highly relevant in the activation of MFB. In this sense, hepatic macrophages have been described as a potential target for the treatment of liver fibrosis. In vitro and in vivo studies described that both Kupffer cells and monocyte-derived macrophages can activate HSC and induce their trans-differentiation by paracrine mechanisms, including by TGF-β. Resident hepatic macrophages secrete the chemokine CCL2 (a potent chemoattractant) in order to recruit monocytes which could increase and promote fibrosis. Macrophages are essential players in the regulation of liver fibrosis and are an important source of TGF-β. Recent observations have indicated a role for TGF-β in the induction of fibrosis-promoting M2-like macrophage polarization via SNAIL. M2-activation/polarization has a relevant role in the development of fibrosis in mice and patients with liver fibrosis. Thus, in some embodiments, TGF-β's role is altered by applying one or more of the anti-Gal3 antibodies or binding fragments thereof provided herein, which disrupts the interaction between Gal3 and TGF-β receptors, thereby altering one or more of the pathways or processes described above.

In some embodiments, the methods involve an antibody or binding fragment thereof that binds to Gal3, and disrupts an interaction between Gal3 and a cell surface marker or cell surface receptor. In some embodiments, the cell surface marker or cell surface receptor is a cell surface marker or cell surface receptor that appears on a tumor cell, immune cell, cancer cell, or fibrotic cell. This can be a direct obstruction of the interaction zone between Gal3 and the cell surface marker or cell surface receptor, or an indirect alteration, such as a binding that results in a conformational change of Gal3, so that it no longer binds or is active with the cell surface marker or cell surface receptor. It can also result by binding to a first section of Gal3, where some other part of the antibody obstructs or alters Gal3's interaction with the cell surface marker or cell surface receptor.

In some embodiments, the method involves an antibody that binds to Gal3, and disrupts an interaction between Gal3 and TGF-β receptors, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof. This can be a direct obstruction of the interaction zone between Gal3 and the TGF-β receptors, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof; or an indirect alteration, such as a binding that results in a conformational change of Gal3, so that it no longer binds or is active with the TGF-β receptors, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof. It can also result by binding to a first section of Gal3, where some other part of the antibody obstructs or alters Gal3 s interaction with the TGF-β receptors, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof.

In some embodiments, any of the anti-Gal3 antibodies or binding fragments thereof or any arrangement of any of the anti-Gal3 antibodies or binding fragments provided herein may be substituted with an antigen binding molecule that binds to Gal3. Thus, the present disclosure also envisions antigen binding molecules each time it mentions antibodies or binding fragments thereof, but for brevity, the present disclosure sometimes simply refers to antibodies or binding fragments thereof. It is noted that the term “antigen binding molecule” encompasses antibodies and binding fragments thereof and denotes a broader genus of options.

The present disclosure claims priority to one or more priority documents, which may have been filed with one or more appendices. All subject matter disclosed in the priority documents and any appendices are hereby expressly contemplated as part of the disclosure in this document as further embodiments that can be combined and/or modified with any of the embodiments provided herein. All subject matter disclosed in the priority documents and appendices, including but not limited to antibodies, binding fragments thereof, antigen binding molecules, and any methods, such as methods of making, methods of use, or methods of treatment may be applied to any embodiment or arrangement as disclosed in this section of the application. Similarly, all subject matter disclosed herein, including but not limited to antibodies, binding fragments thereof, antigen binding molecules, and any methods, such as methods of making, methods of use, or methods of treatment are contemplated to be applied to any embodiment or arrangement as disclosed in the priority documents and appendices.

### Definitions

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

The section headings used herein are for organizational purposes only and are not to be construed as limiting the subject matter described.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

The terms “polypeptide”, “peptide”, and “protein” are used interchangeably herein to refer to polymers of amino acids of any length. The polymer may be linear, cyclic, or branched, it may comprise modified amino acids, and it may be interrupted by non-amino acids. The terms also encompass amino acid polymers that have been modified, for example, via sulfation, glycosylation, lipidation, acetylation, phosphorylation, iodination, methylation, oxidation, proteolytic processing, phosphorylation, prenylation, racemization, selenoylation, transfer-RNA mediated addition of amino acids to proteins such as arginylation, ubiquitination, or any other manipulation, such as conjugation with a labeling component.

As used herein the term “amino acid” refers to either natural and/or unnatural or synthetic amino acids, including glycine and both the D or L optical isomers, and amino acid analogs and peptidomimetics.

A polypeptide or amino acid sequence “derived from” a designated protein refers to the origin of the polypeptide. Preferably, the polypeptide has an amino acid sequence that is essentially identical to that of a polypeptide encoded in the sequence, or a portion thereof wherein the portion consists of at least 10-20 amino acids, or at least 20-30 amino acids, or at least 30-50 amino acids, or which is immunologically identifiable with a polypeptide encoded in the sequence. This terminology also includes a polypeptide expressed from a designated nucleic acid sequence. Peptide sequences having at least 80%, 85%, 90%, 95%, 99%, or 100% homology to any one of the peptide sequences disclosed herein and having the same or similar functional properties are envisioned. The percent homology may be determined according to amino acid substitutions, deletions, or additions between two peptide sequences. Peptide sequences having some percent homology to any one of the peptide sequences disclosed herein may be produced and tested by one skilled in the art through conventional methods.

As used herein, the term “antibody” denotes the meaning ascribed to it by one of skill in the art, and further it is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly and can be modified to reduce their antigenicity.

In addition to entire immunoglobulins (or their recombinant counterparts), immunoglobulin fragments or “binding fragments” comprising the epitope binding site (e.g., Fab′, F(ab′)₂, single-chain variable fragment (scFv), diabody, minibody, nanobody, single-domain antibody (sdAb), or other fragments) are useful as antibody moieties in the present invention. Such antibody fragments may be generated from whole immunoglobulins by ricin, pepsin, papain, or other protease cleavage. Minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif). Nanobodies or single-domain antibodies can also be derived from alternative organisms, such as dromedaries, camels, llamas, alpacas, or sharks. In some embodiments, antibodies can be conjugates, e.g. pegylated antibodies, drug, radioisotope, or toxin conjugates. Monoclonal antibodies directed against a specific epitope, or combination of epitopes, will allow for the targeting and/or depletion of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (e.g. U.S. Pat. No. 5,985,660, hereby expressly incorporated by reference in its entirety).

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3. As is known in the art, an Fc region can be present in dimer or monomeric form.

As known in the art, a “constant region” of an antibody refers to the constant region of the antibody light chain or the constant region of the antibody heavy chain, either alone or in combination.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the IMGT approach (Lefranc et al., 2003) Dev Comp Immunol. 27:55-77), computational programs such as Paratome (Kunik et al., 2012, Nucl Acids Res. W521-4), the AbM definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, IMGT, Paratome, AbM, and/or conformational definitions, or a combination of any of the foregoing.

The term “compete,” as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, and/or more rapidly, and/or with greater duration and/or with greater affinity with a particular cell or substance than it does with alternative cells or substances. An antibody “specifically binds” or “preferentially binds” to a target if it binds with greater affinity, and/or avidity, and/or more readily, and/or with greater duration than it binds to other substances. For example, an antibody that specifically or preferentially binds to a CFD epitope is an antibody that binds this epitope with greater affinity, and/or avidity, and/or more readily, and/or with greater duration than it binds to other CFD epitopes or non-CFD epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, the term “antigen binding molecule” refers to a molecule that comprises an antigen binding portion that binds to an antigen and, optionally, a scaffold or framework portion that allows the antigen binding portion to adopt a conformation that promotes binding of the antigen binding portion or provides some additional properties to the antigen binding molecule. In some embodiments, the antigen is Gal3. In some embodiments, the antigen binding portion comprises at least one CDR from an antibody that binds to the antigen. In some embodiments, the antigen binding portion comprises all three CDRs from a heavy chain of an antibody that binds to the antigen or from a light chain of an antibody that binds to the antigen. In some embodiments, the antigen binding portion comprises all six CDRs from an antibody that binds to the antigen (three from the heavy chain and three from the light chain). In some embodiments, the antigen binding portion is an antibody fragment.

Non-limiting examples of antigen binding molecules include antibodies, antibody fragments (e.g., an antigen binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment, see Cortez-Retamozo et al., Cancer Research, Vol. 64:2853-57, 2004), a Fab fragment, a Fab′ fragment, a F(ab′)2 fragment, a Fv fragment, a Fd fragment, and a complementarity determining region (CDR) fragment. These molecules can be derived from any mammalian source, such as human, mouse, rat, rabbit, pig, dog, cat, horse, donkey, guinea pig, goat, or camelid. Antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis. The antigen binding molecule can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding molecule as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronectin components as a scaffold.

An antigen binding molecule can also include a protein comprising one or more antibody fragments incorporated into a single polypeptide chain or into multiple polypeptide chains. For instance, antigen binding molecule can include, but are not limited to, a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (V_{H}-CH1-V_{H}-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, V_{H}-IgG, IgG-V_{H}, and Fab-scFv-Fc).

In certain embodiments, an antigen binding molecule can have, for example, the structure of an immunoglobulin. An “immunoglobulin” is a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

As used herein, the terms “treating” or “treatment” (and as well understood in the art) means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the recurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the subject. The length of the treatment period depends on a variety of factors, such as the severity of the condition, the age and genetic profile of the subject, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The terms “effective amount” or “effective dose” as used herein have their plain and ordinary meaning as understood in light of the specification, and refer to that amount of a recited composition or compound that results in an observable designated effect. Actual dosage levels of active ingredients in an active composition of the presently disclosed subject matter can be varied so as to administer an amount of the active composition or compound that is effective to achieve the designated response for a particular subject and/or application. The selected dosage level can vary based upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriol, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a first compound described herein is administered at the same time, just prior to, or just after the administration of a second compound described herein.

As used herein, the term “therapeutic target” refers to a gene or gene product that, upon modulation of its activity (e.g., by modulation of expression, biological activity, and the like), can provide for modulation of the disease phenotype. As used throughout, “modulation” is meant to refer to an increase or a decrease in the indicated phenomenon (e.g., modulation of a biological activity refers to an increase in a biological activity or a decrease in a biological activity).

As used herein, the term “standard of care”, “best practice” and “standard therapy” refers to the treatment that is accepted by medical practitioners to be an appropriate, proper, effective, and/or widely used treatment for a certain disease. The standard of care of a certain disease depends on many different factors, including the biological effect of treatment, region or location within the body, patient status (e.g. age, weight, gender, hereditary risks, other disabilities, secondary conditions), toxicity, metabolism, bioaccumulation, therapeutic index, dosage, and other factors known in the art. Determining a standard of care for a disease is also dependent on establishing safety and efficacy in clinical trials as standardized by regulatory bodies such as the US Food and Drug Administration, International Council for Harmonisation, Health Canada, European Medicines Agency, Therapeutics Goods Administration, Central Drugs Standard Control Organization, National Medical Products Administration, Pharmaceuticals and Medical Devices Agency, Ministry of Food and Drug Safety, and the World Health Organization. The standard of care for a disease may include but is not limited to surgery, radiation, chemotherapy, targeted therapy, or immunotherapy (e.g. PD1/PDL1 or CTLA4 blockade therapy). For example, temozolomide is an orally administered chemotherapy compound used as the standard of care treatment for brain cancers such as glioblastoma and astrocytoma. One skilled in the art will appreciate that the ability for temozolomide to cross the blood-brain barrier is one aspect that determines its utility as a standard of care for these diseases, and also that temozolomide may not necessarily be used as a standard of care treatment for other diseases.

As used herein, the term “supplement” refers to a compound, molecule, or substance that imparts an effect on a patient that is provided in conjunction with at least one other compound, molecule, or substance to treat cancer. The term “immuno-oncology supplement” refers to a supplement that imparts an effect on the immune system of the patient. The administration of these at least two compounds, molecules, or substances can also be referred to as a combination therapy. In some embodiments, at least one other compound, molecule, or substance is a PD1 blockade therapy, a PDL1 blockade therapy, or a CTLA4 blockade therapy.

As used herein, “PD1 blockade therapy” refers to PD1 inhibitor therapeutics involved in blocking the interaction between programmed cell death protein 1 (PD1) and programmed death-ligand 1 (PDL1). Cancer cells express PDL1, which bind to PD1 expressed on T cells or other immune cells to inhibit immune clearance of the cancer cell. PD1 inhibitors block this interaction by binding to or inhibiting PD1. PD1 inhibitors include but are not limited to pembrolizumab, nivolumab, cemiplimab, spartalizumab, camrelizumab, sintilimab, tislelizumab, toripalimab, AMP-224, or AMP-514, or any combination thereof. As used herein, “PDL1 blockade therapy” refers to PDL1 inhibitor therapeutics which behave similarly to PD1 inhibitors. PDL1 inhibitors bind to or inhibit PDL1. PDL1 inhibitors include but are not limited to atezolizumab, avelumab, durvalumab, KN035, CK-301, AUNP12, CA-170, or BMS-986189, or any combination thereof. “PD1/PDL1 blockade therapies” refer to a PD1 blockade therapy, a PDL1 blockade therapy, or both. As used herein, “CTLA4 blockade therapy” refers to CTLA4 inhibitor therapeutics involved in blocking the interaction between cytotoxic T-lymphocyte-associated protein 4 (CTLA4) and CD80 or CD86. T cells express CTLA4, which bind to CD80 or CD86 on other T cells to inhibit their immune activity. CTLA4 inhibitors include but are not limited to ipilimumab or tremilimumab. PD1 blockade therapies, PDL1 blockade therapies, and/or CTLA4 blockade therapies are used as a standard of care treatment for some cancers or other diseases.

As used herein, the term “neurological disorder” refers to a disease affecting the central and/or peripheral nervous system of a patient. A neurological disorder has a physical cause, such as external or internal mechanical trauma (e.g. stroke or concussion), biological trauma (e.g. infection), chemical trauma (e.g. toxins or drugs), aging and age-related senescence, genetics, and many other causes. Some neurological disorders are caused by the effect or accumulation of mutated or misfolded proteins. These diseases may involve the death of neurons or other cell types associated with the nervous system. Non-limiting examples of neurological disorders include inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or otherwise known by a person skilled in the art. Some neurological disorders can also be categorized as proteopathies.

As used herein, the term “proteopathy” refers to a disease which is caused by abnormal folding or accumulation of proteins. An abnormal protein may gain a toxic function, or lose their normal function. It is possible that misfolded proteins can induce the misfolding of otherwise normally folded proteins, resulting in an amplification of the disease (e.g. prion disease). Some non-limiting examples of proteopathies include Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or otherwise known by a person skilled in the art.

As used herein, the terms “amyloid-beta”, “amyloid-D” and “AD” have their plain and ordinary meaning as understood in light of the specification and refer to amyloid-O proteins or peptides, amyloid R precursor proteins or peptides, intermediates, and modifications and fragments thereof, unless otherwise specifically indicated. In particular, “AD” refers to any peptide produced by proteolytic processing of the amyloid precursor protein (APP) gene product, especially peptides which are associated with amyloid pathologies.

As used herein, the term “blood-brain barrier” has its plain and ordinary meaning as understood in light of the specification and refers to the protective cellular boundary between the circulatory system and central nervous system. This boundary is comprised of closely interacting brain capillary endothelial cells (BCECs) of the associated capillary vessels through tight junctions, which exhibit selectivity for different small and large molecules in addition to larger particles such as circulating immune cells and pathogenic organisms. Generally, small polar molecules or hydrophobic molecules are able to naturally diffuse through the blood-brain barrier, but larger and/or more polar molecules (e.g. glucose, proteins) require specific transporters expressed by the endothelial cells to be able to cross the barrier. Some antibodies have been shown to be able to cross the blood-brain barrier by having specificity towards a cell receptor or transporter on the endothelial cells, which are internalized and undergo transcytosis. The BBB functions as a physical, metabolic and immunological barrier. As disclosed herein in some embodiments, the antibodies or binding fragments thereof disclosed herein may be able to cross the blood-brain barrier of a subject. In some embodiments, the subject may have an intact blood-brain barrier. In some embodiments, the subject may have a damaged or improperly functioning blood-brain barrier. In some embodiments, the damaged or improperly functioning blood-brain barrier is due to a neurodegenerative disease, including but not limited to Alzheimer's disease, or associated with a brain cancer, such as primary and/or secondary brain tumor-associated damage.

As used herein, the term “neuronal regeneration” has its plain and ordinary meaning as understood in light of the specification and refers to new growth of cells or components thereof associated with the nervous system. For example, regeneration can occur with neurons, glia, oligodendrocytes, astrocytes, ependymal cells, microglia, or components thereof such as axons, dendrites, myelin, or development of new synapses/neuronal interactions. While regeneration of neuronal tissue is generally much slower than other tissues in adults, some repair does occur upon damage or injury. While there are currently no treatments to enhance neuronal regeneration, research is being done towards treatments and prophylaxes, such as preventing neurodegeneration in diseases such as Alzheimer's disease and multiple sclerosis. As disclosed herein in some embodiments, the antibodies or binding fragments thereof disclosed herein may be able to promote neuronal regeneration.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

The term “tumor microenvironment” refers to a cellular environment in which the tumor exists, including tumor cells and surrounding blood vessels, immune cells, fibroblasts, bone marrow-derived inflammatory cells, lymphocytes, signaling molecules and the extracellular matrix.

The term “immune cells” refers to cells of hematopoietic origin that are involved in the specific recognition of antigens. Immune cells include antigen presenting cells (APCs), such as dendritic cells or macrophages, B cells, T cells, natural killer cells, and myeloid cells, such as monocytes, macrophages, eosinophils, mast cells, basophils, and granulocytes.

The term “immune response” refers to T cell-mediated and/or B cell-mediated immune responses. Exemplary immune responses include B cell responses (e.g., antibody production), T cell responses (e.g., cytokine production, and cellular cytotoxicity) and activation of cytokine responsive cells, e.g., macrophages. The term “activating immune response” refers to enhancing the level of T-cell-mediated and/or B cell-mediated immune response, using methods known to one skilled in the art. In one embodiment, the level of enhancement is at least 20-50%, alternatively at least 60%, at least 70%, at least 80%, at least 90%, at least 100%, at least 120%, at least 150%, or at least 200%.

As used herein, the term “transforming growth factor beta receptor” (TGF-b receptor, or TGF-β receptor) refers to a family of serine/threonine kinase receptors expressed on cell surfaces that are specific for the protein transforming growth factor beta (TGF-b, TGF-β). The interaction between TGF-b and the receptor triggers a signaling pathway that is responsible for many functions, including but not limited to cell growth, differentiation (e.g. stem cells, immune cells), apoptosis, homeostasis, chemotaxis, inflammation, and immune cell activation. The TGF-b receptor family includes TGF-b receptor type 1 (TGFbR1), TGF-b receptor type 2 (TGFbR2), and TGF-b receptor type 3 (TGFbR3).

As used herein, the term “vascular endothelial growth factor receptor” (VEGFR) refers to a family of tyrosine kinase receptors specific for vascular endothelial growth factor (VEGF). The VEGFR family includes VEGFR1, VEGFR2, and VEGFR3.

As used herein, the term “epidermal growth factor receptor” (EGFR, ErbB1, HER1) refers to a tyrosine kinase receptor specific for epidermal growth factor (EGF) and transforming growth factor α (TGFα) belonging to the ErbB family of tyrosine kinases.

As used herein, the term “platelet-derived growth factor receptor” (PDGFR) refers to a family of tyrosine kinase receptors specific for platelet-derived growth factor (PDGF). The PDGFR family includes PDGFR alpha (PDGFRa, PDGFRα) and PDGFR beta (PDGFRb, PDGFRβ)

As used herein, the term “HER2/neu” (ErbB2, HER2) refers to a tyrosine kinase receptor belonging to the ErbB family of tyrosine kinases.

As used herein, the term “hepatocyte growth factor receptor” and “tyrosine-protein kinase Met” (HGFR, cMet, c-Met) refers to a tyrosine kinase receptor specific for hepatocyte growth factor/scatter factor (HGF/SF).

As used herein, the term “tumor necrosis factor soluble receptor I” (TNF sRI) refers to the soluble fragment of TNF-u after proteolytic cleavage by TNF-α converting enzyme.

As used herein, the term “integrin associated protein” (CD47, IAP) refers to a transmembrane surface signaling protein belonging to the immunoglobulin superfamily.

As used herein, the term “fibroblast growth factor receptor” (FGFR) refers to a family of tyrosine kinase receptors specific for fibroblast growth factors (FGF). The FGFR family includes FGFR1 alpha-IIb, FGFR1 alpha-IIIc, FGFR2 alpha-IIIc, FGFR3 IIIc, and FGFR4.

As used herein, the term “fibrosis” refers to the medical condition wherein tissues or organs harden or scar as a result of unregulated production of extracellular matrix, such as collagen proteins. Fibrosis has been associated with chronic inflammation, where immune cells such as macrophages signal fibroblasts to express extracellular matrix proteins in response. This signaling is achieved through pathways such as growth receptor pathways including but not limited to the TGF-b, EGFR, PDGFR, FGFR, VEGFR, or cMet pathway, although there are other pro-fibrotic pathways as well. Fibrosis includes but is not limited to liver fibrosis, bridging fibrosis, cirrhosis, kidney fibrosis, pulmonary fibrosis, idiopathic pulmonary fibrosis, cystic fibrosis, cardiovascular fibrosis, arterial fibrosis, venous thrombosis, arthrofibrosis, Crohn's disease, Dupuytren's contracture, keloids, mediastinal fibrosis, myelofibrosis, Peyronie's disease, nephrogenic systemic fibrosis, progressive massive fibrosis, retroperitoneal fibrosis, or systemic sclerosis.

As used herein, the term “non-alcoholic fatty liver disease” (NAFLD) refers to fat accumulation in the liver as a result of causes other than alcohol use. A more severe form of NAFLD is “non-alcoholic steatohepatitis” (NASH), which is further defined by inflammation and fibrosis of the liver. NAFLD and NASH can eventually lead to cirrhosis, liver cancer, liver failure, or cardiovascular disease.

As used herein, the term “sepsis” refers to a condition marked by an extreme inflammatory immune response to a pathogenic infection. As used herein, the term “atopic dermatitis” (eczema) refers to an autoimmune condition marked by inflammation of the skin, causing redness, itching, and rashes. As used here, the term “psoriasis” refers to an autoimmune condition marked by inflammation of the skin, resulting in patches of redness, itching, dryness, and rashes on the skin.

The term “% w/w” or “% wt/wt” means a percentage expressed in terms of the weight of the ingredient or agent over the total weight of the composition multiplied by 100.

### Exemplary Anti-Gal3 Antibodies

Unless otherwise specified, the complementarity defining regions disclosed herein follow the IMGT definition. In some embodiments, the CDRs can instead by Kabat, Chothia, or other definitions accepted by those of skill in the art.

It is understood that an antibody with an antibody name described herein can be referred using a shortened version of the antibody name, as long as there are no conflicts with another antibody described herein. For example, 2D10.2B2 may be referred to as 2D10.

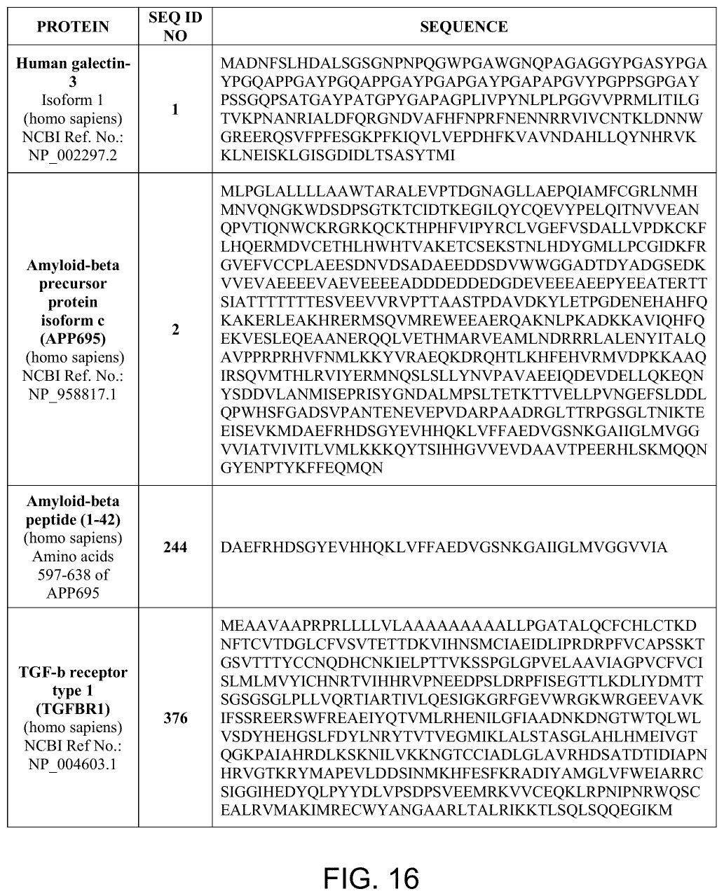

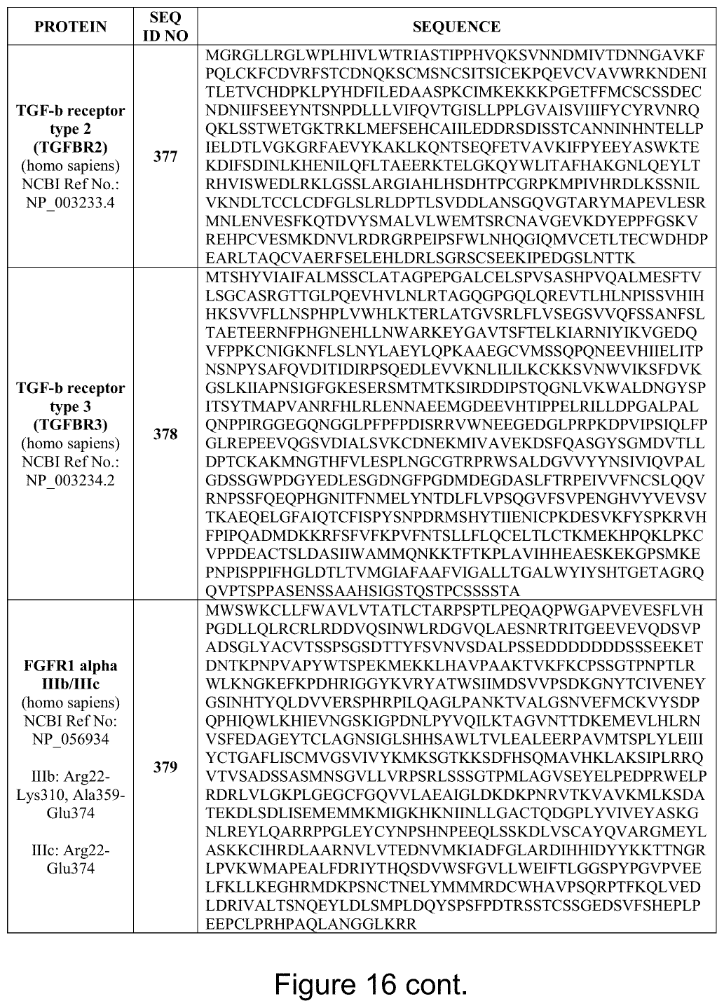

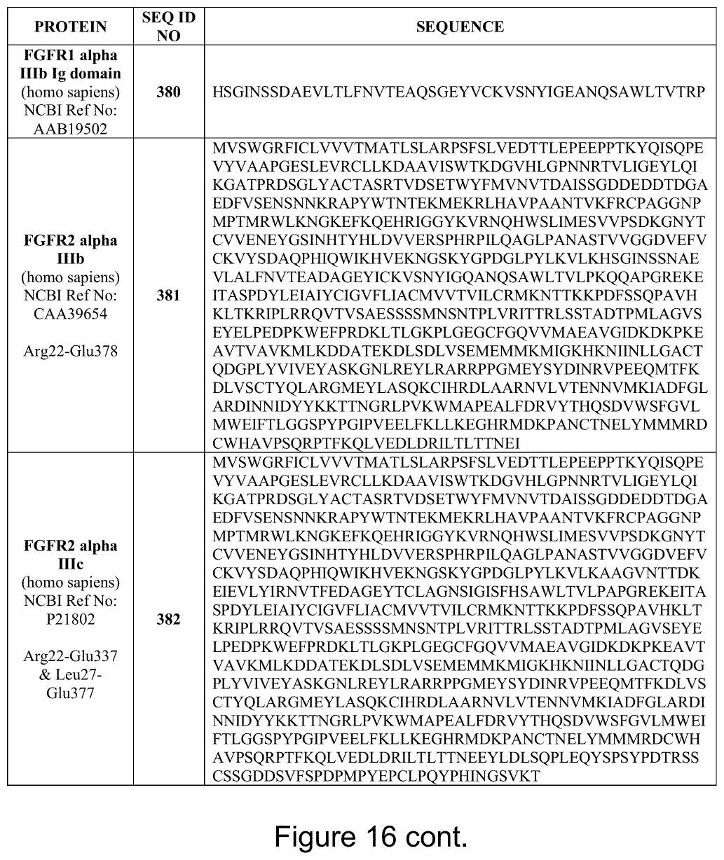

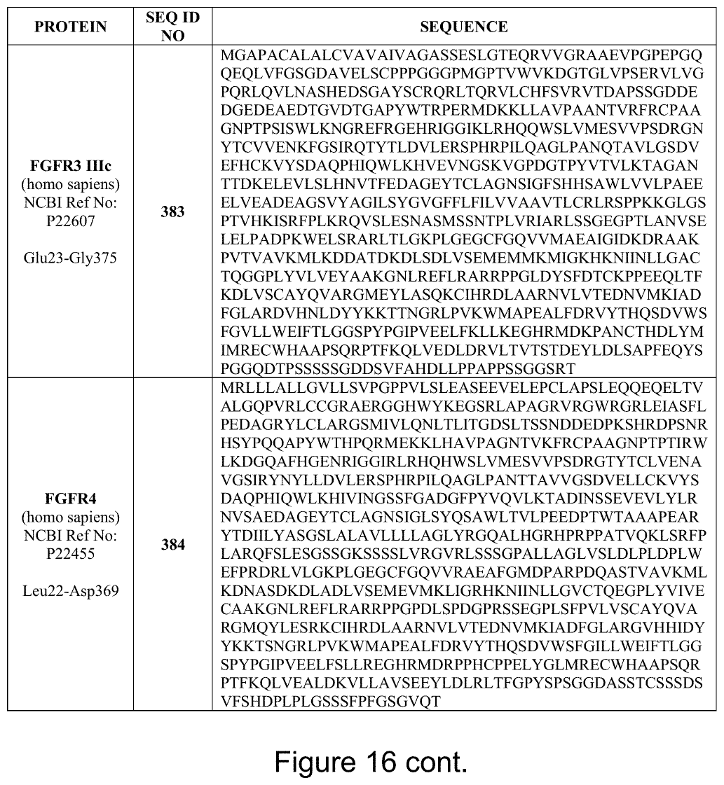

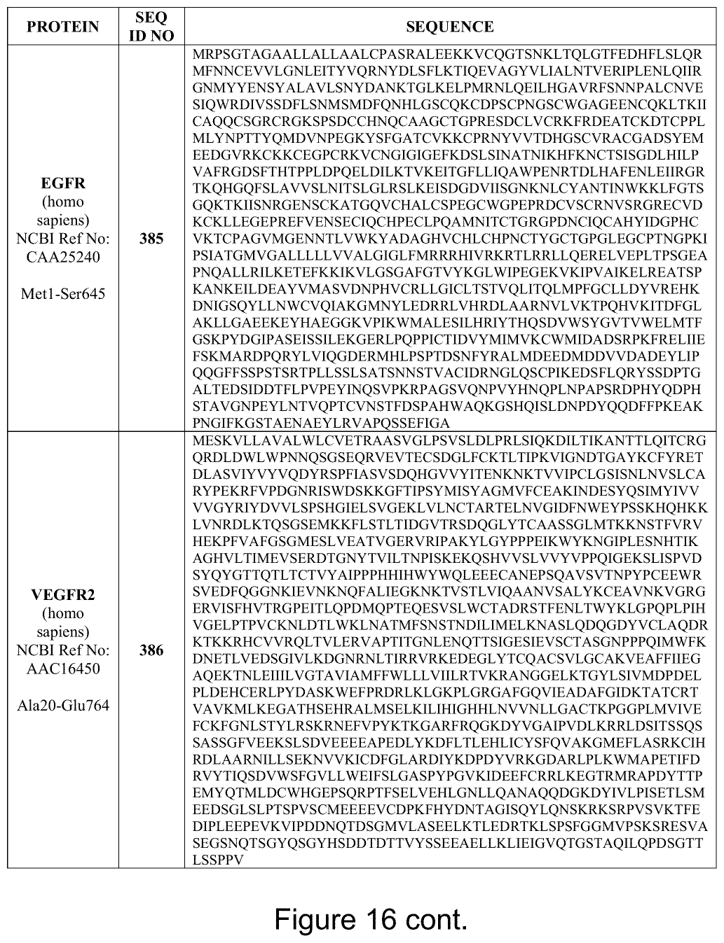

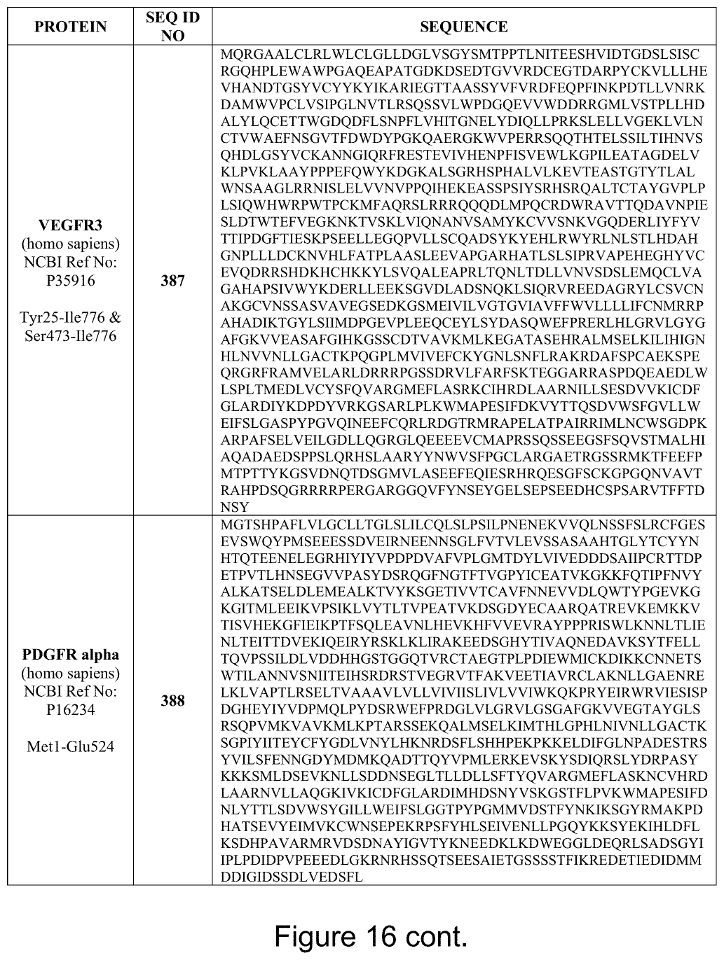

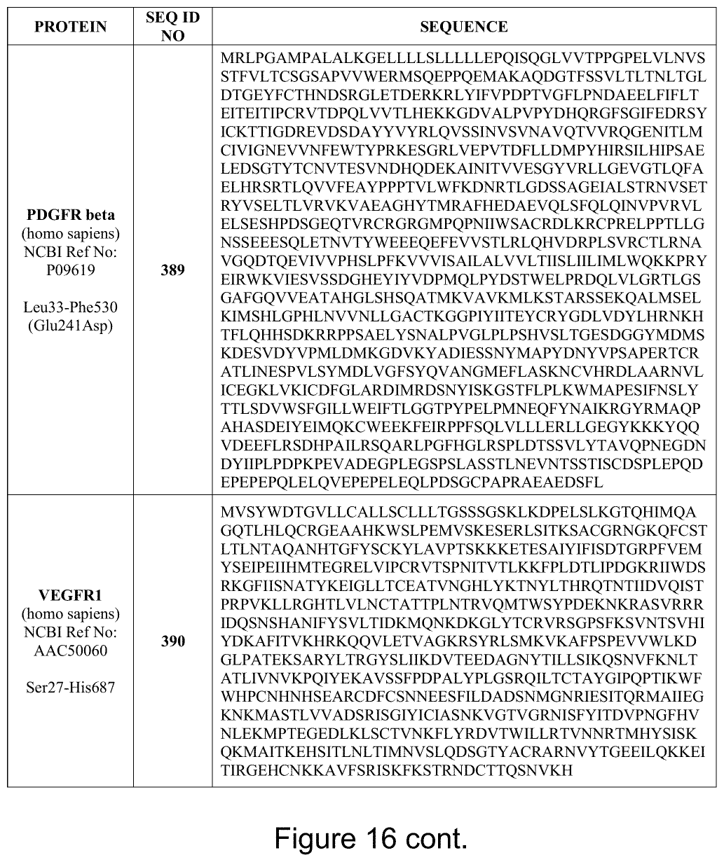

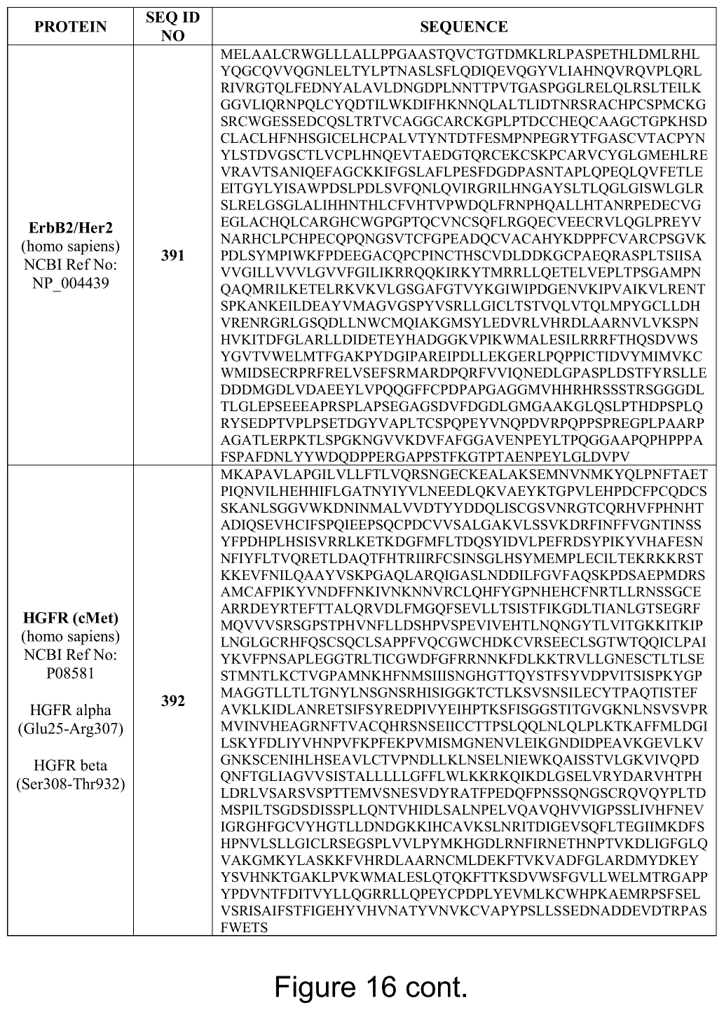

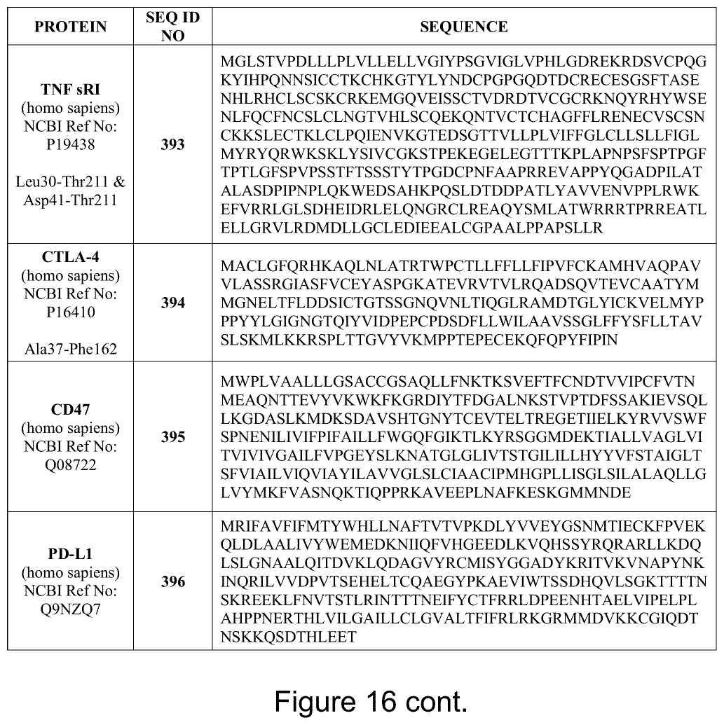

In some embodiments, the anti-Gal3 antibody or binding fragment thereof binds to specific epitopes within a Gal3 protein. In some cases, the anti-Gal3 antibody or binding fragment thereof binds to a specific epitope within a Gal3 protein having an amino acid sequence according to SEQ ID NO: 1, provided in FIG. 16.

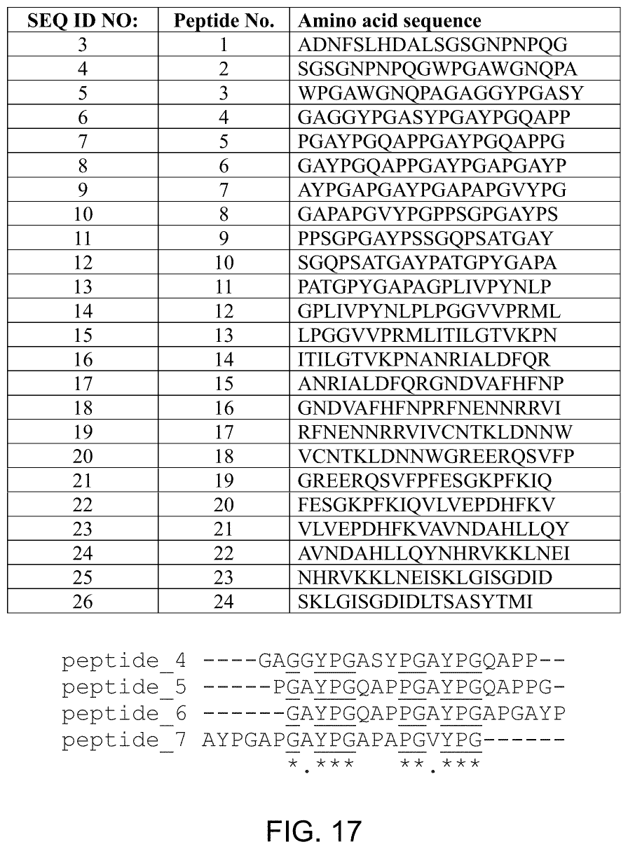

In some instances, the anti-Gal3 antibody or binding fragment thereof may bind to at least 1, 2, 3, 4, 5, 6, 10, 15, or 20 amino acid residues within a peptide illustrated in FIG. 17.

In some embodiments, the anti-Gal3 antibody or binding fragment thereof may bind to at least 1, 2, 3, 4, 5, 6, 10, 15, or 20 amino acid residues within amino acid residues 1-20 of SEQ ID NO: 1. In some embodiments, the anti-Gal3 antibody or binding fragment thereof may bind to at least 1, 2, 3, 4, 5, 6, 10, 15, or 20 amino acid residues within amino acid residues 31-50 of SEQ ID NO: 1. In some embodiments, the anti-Gal3 antibody or binding fragment thereof may bind to at least 1, 2, 3, 4, 5, 6, 10, 15, or 20 amino acid residues within amino acid residues 51-70 of SEQ ID NO: 1. In some embodiments, the anti-Gal3 antibody or binding fragment thereof may bind to at least 1, 2, 3, 4, 5, 6, 10, 15, or 20 amino acid residues within amino acid residues 61-80 of SEQ ID NO: 1. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some instances, the anti-Gal3 antibody or binding fragment thereof may bind to at least 1, 2, 3, 4, 5, 6, 7, 8, 9, 10, 11, 12, 13, 14, 15, 16, 17, 18, 19, or 20 amino acid residues within Peptide 1 (SEQ ID NO: 3), Peptide 2 (SEQ ID NO: 4), Peptide 3 (SEQ ID NO: 5), Peptide 4 (SEQ ID NO: 6), Peptide 5 (SEQ ID NO: 7), Peptide 6 (SEQ ID NO: 8), Peptide 7 (SEQ ID NO: 9), Peptide 8 (SEQ ID NO: 10), or Peptide 17 (SEQ ID NO: 19) or any combination thereof. In some embodiments, the anti-Gal3 or binding fragment thereof may bind to at least 1, 2, 3, 4, 5, 6, 7, 8, 9, 10, 11, 12, 13, 14, 15, 16, 17, 18, 19, or 20 amino acid residues within Peptide 6 (SEQ ID NO: 8). In some embodiments, any of the anti-Gal3 antibodies or binding fragments thereof or any arrangement of any of the anti-Gal3 antibodies or binding fragments provided herein may be substituted with an antigen binding molecule that binds to Gal3.

Some exemplary antibodies that bind to Peptide 1 (SEQ ID NO: 3) are 23H9.2E4, F846C.1H5, F846TC.14A2, F846TC.7F10, F847C.10B9, F847C.12F12, F847C.26F5, and F847C.4B10.

Some exemplary antibodies that bind to Peptide 2 (SEQ ID NO: 4) are 15F10.2D6, 7D8.2D8, F846TC.14E4, F849C.8D10, and F849C.8H3.

Some exemplary antibodies that bind to Peptide 3 (SEQ ID NO: 5) are 15F10.2D6, 7D8.2D8, and F849C.8D10.

Some exemplary antibodies that bind to Peptide 4 (SEQ ID NO: 6) are 13A12.2E5 and 15F10.2D6.

Some exemplary antibodies that bind to Peptide 5 (SEQ ID NO: 7) are F846C.1B2 and F846C.1H12.

Some exemplary antibodies that bind to Peptide 6 (SEQ ID NO: 8) are 13A12.2E5, 14H10.2C9, 23H9.2E4, F846C.1B2, F846C.1F5, F846C.1H12, F846C.1H12, F846C.2H3, and F846TC.16B5.

Some exemplary antibodies that bind to Peptide 7 (SEQ ID NO: 9) are 14H10.2C9, 23H9.2E4, F846C.1B2, F846TC.14A2, F847C.10B9, F847C.12F12, and F847C.26F5.

Some exemplary antibodies that bind to Peptide 8 (SEQ ID NO: 10) are 23H9.2E4 and F846TC.14A2.

Some exemplary antibodies that bind to Peptide 17 (SEQ ID NO: 19) are 7D8.2D8, F846C.1F5, F846C.1H12, F846TC.16B5, F847C.11B1, and F849C.8H3.

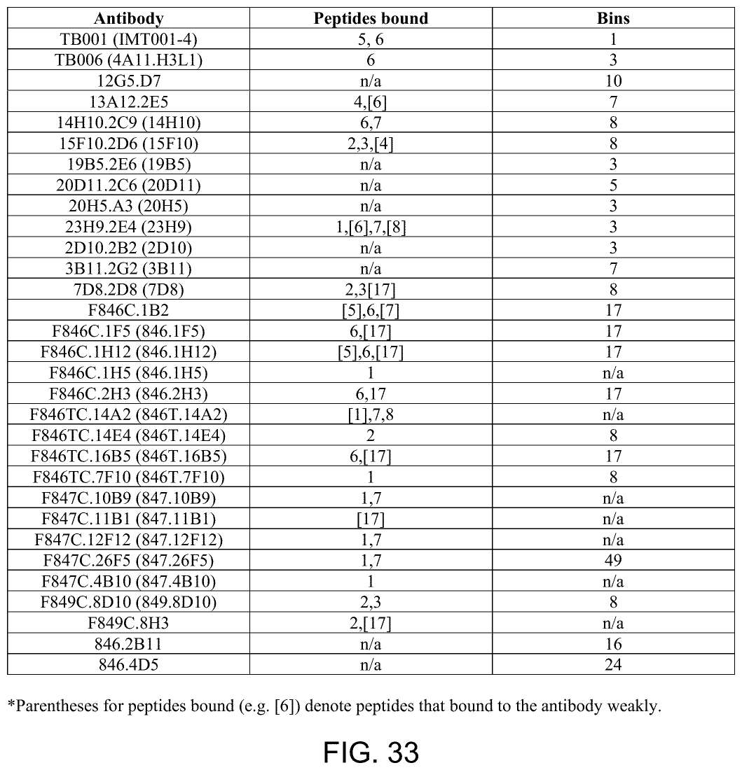

In some embodiments, the anti-Gal3 antibody or binding fragment thereof are epitope binned. Epitope bins of some exemplary antibodies are depicted in FIG. 33. An exemplary binning process is detailed in Example 3.

In some embodiments, antibody TB001 is categorized into bin 1.

In some embodiments, antibodies TB006, 19B5.2E6, 20H5.A3, 23H9.2E4, and 2D10.2B2 are categorized into bin 3.

In some embodiments, antibody 20D11.2C6 is categorized into bin 5.

In some embodiments, antibodies 13A12.2E5 and 3B11.2G2 are categorized into bin 7.

In some embodiments, antibodies 14H10.2C9, 15F10.2D6, 7D8.2D8, F846TC.14E4, F846TC.7F10, and F849C.8D10 are categorized into bin 8.

In some embodiments, antibody 12G5.D7 is categorized into bin 10.

In some embodiments, antibody 846.2B11 is categorized into bin 16.

In some embodiments, antibodies F846C.1B2, F846C.1F5, F846C.1H12, F846C.2H3, and F846TC.16B5 are categorized into bin 17.

In some embodiments, antibody 846.4D5 is categorized into bin 24.

In some embodiments, antibodies F847C.10B9, F847C.12F12, and F847C.26F5 are categorized into bin 49. In some embodiments, any antibody that binds with any of the bins provided herein are contemplated.

In some embodiments, an anti-Gal3 antibody or binding fragment thereof as described herein may bind to the N-terminal domain of Gal3 or a portion thereof. In some embodiments, an anti-Gal3 antibody or binding fragment thereof as described herein may bind to an epitope of Gal3 that includes a motif of GxYPG, where x is the amino acids alanine (A), glycine (G), or valine (V). In some embodiments, an anti-Gal3 antibody or binding fragment thereof as described herein may bind to an epitope of Gal3 that includes two GxYPG motifs separated by three amino acids, where x is A, G, or V.

In some embodiments, the anti-Gal3 antibody or binding fragment thereof binds to Gal3. In some embodiments, the anti-Gal3 antibody or binding fragment thereof binds to the N-terminus of Gal3, the N-terminal domain of Gal3, or the TRD of Gal3. In some embodiments, the anti-Gal3 antibody or binding fragment thereof does not bind to the N-terminus of Gal3, the N-terminal domain of Gal3, or the TRD of Gal3. In some embodiments, the anti-Gal3 antibody or binding fragment thereof binds to the C-terminus of Gal3, the C-terminal domain of Gal3, or the CRD of Gal3. In some embodiments, the anti-Gal3 antibody or binding fragment thereof does not bind to the C-terminus of Gal3, the C-terminal domain of Gal3, or the CRD of Gal3.

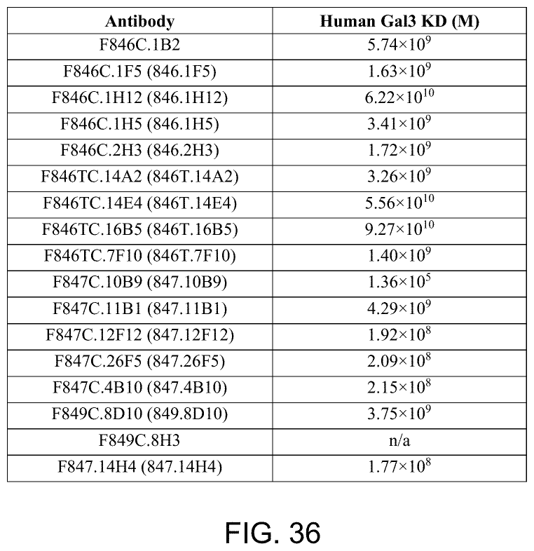

In some instances, the anti-Gal3 antibody or binding fragment thereof binds to Gal3 with a dissociation constant (KD) of less than 1 nM, less than 1.2 nM, less than 2 nM, less than 5 nM, less than 10 nM, less than 13.5 nM, less than 15 nM, less than 20 nM, less than 25 nM, or less than 30 nM. In some instances, the anti-Gal3 antibody or binding fragment thereof binds to Gal3 with a KD of less than 1 nM. In some instances, the anti-Gal3 antibody or binding fragment thereof binds to Gal3 with a KD of less than 1.2 nM. In some instances, the anti-Gal3 antibody or binding fragment thereof binds to Gal3 with a KD of less than 2 nM. In some instances, the anti-Gal3 antibody or binding fragment thereof binds to Gal3 with a KD of less than 5 nM. In some instances, the anti-Gal3 antibody or binding fragment thereof binds to Gal3 with a KD of less than 10 nM. In some instances, the anti-Gal3 antibody or binding fragment thereof binds to Gal3 with a KD of less than 13.5 nM. In some instances, the anti-Gal3 antibody or binding fragment thereof binds to Gal3 with a KD of less than 15 nM. In some instances, the anti-Gal3 antibody or binding fragment thereof binds to Gal3 with a KD of less than 20 nM. In some instances, the anti-Gal3 antibody or binding fragment thereof binds to Gal3 with a KD of less than 25 nM. In some instances, the anti-Gal3 antibody or binding fragment thereof binds to Gal3 with a KD of less than 30 nM. KD values of Gal3 binding of exemplary anti-Gal3 antibodies are provided in FIG. 36. In some embodiments, any of the methods disclosed herein involving an anti-Gal3 antibody or binding fragment can be performed with an antigen binding molecule that binds to Gal3.

Disclosed herein are anti-Gal3 antibodies or binding fragments thereof with specific sequences. In some embodiments, the anti-Gal3 antibody or binding fragment thereof comprises (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 27-44, 245-246, 397-399, 588-615, the V_{H}-CDR2 comprises an amino acid sequence selected from SEQ ID NOs: 45-60, 247-248, 400-406, 616-643, the V_{H}-CDR3 comprises an amino acid sequence selected from SEQ ID NOs: 61-81, 249-250, 407-416, 644-671, the V_{L}-CDR1 comprises an amino acid sequence selected from SEQ ID NOs: 82-101, 251-252, 417-426, 672-699, the V_{L}-CDR2 comprises an amino acid sequence selected from SEQ ID NOs: 102-116, 253, 427-428, 700-727, and the V_{L}-CDR3 comprises an amino acid sequence selected from SEQ ID NOs: 117-135, 254-255, 429-434, 728-755. In some embodiments, any of the anti-Gal3 antibodies or binding fragments thereof or any arrangement of any of the anti-Gal3 antibodies or binding fragments provided herein may be substituted with an antigen binding molecule that binds to Gal3.

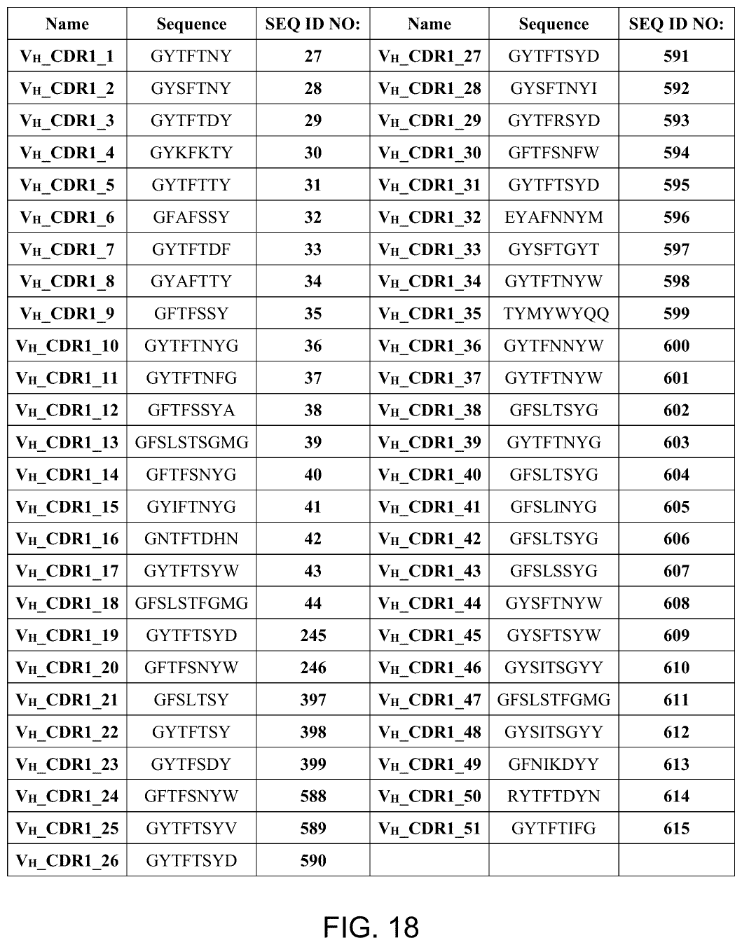

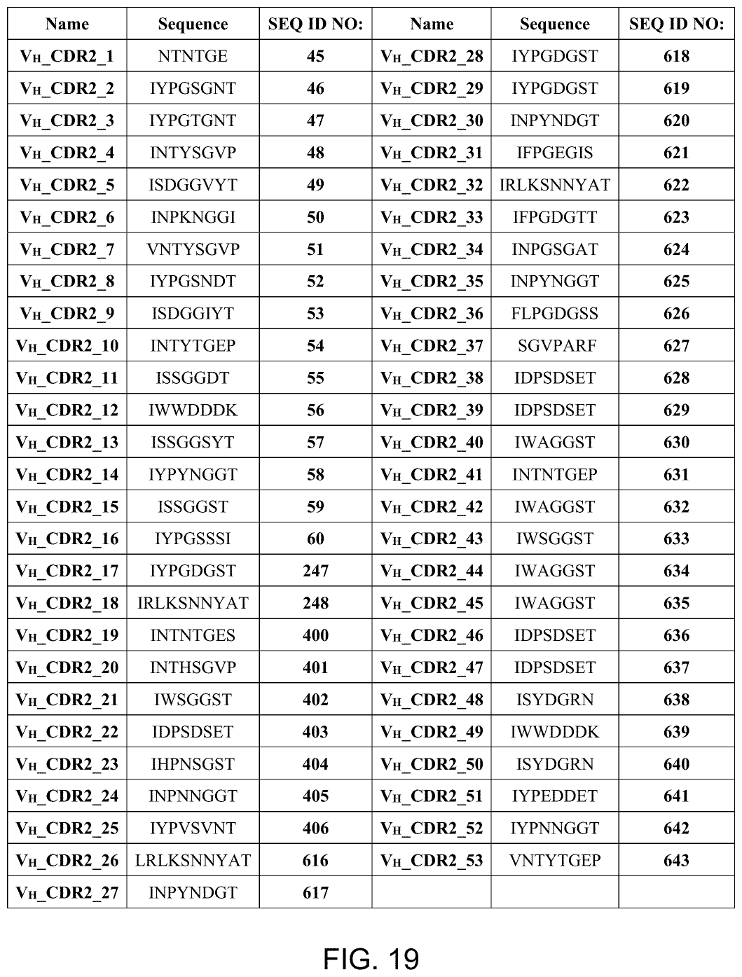

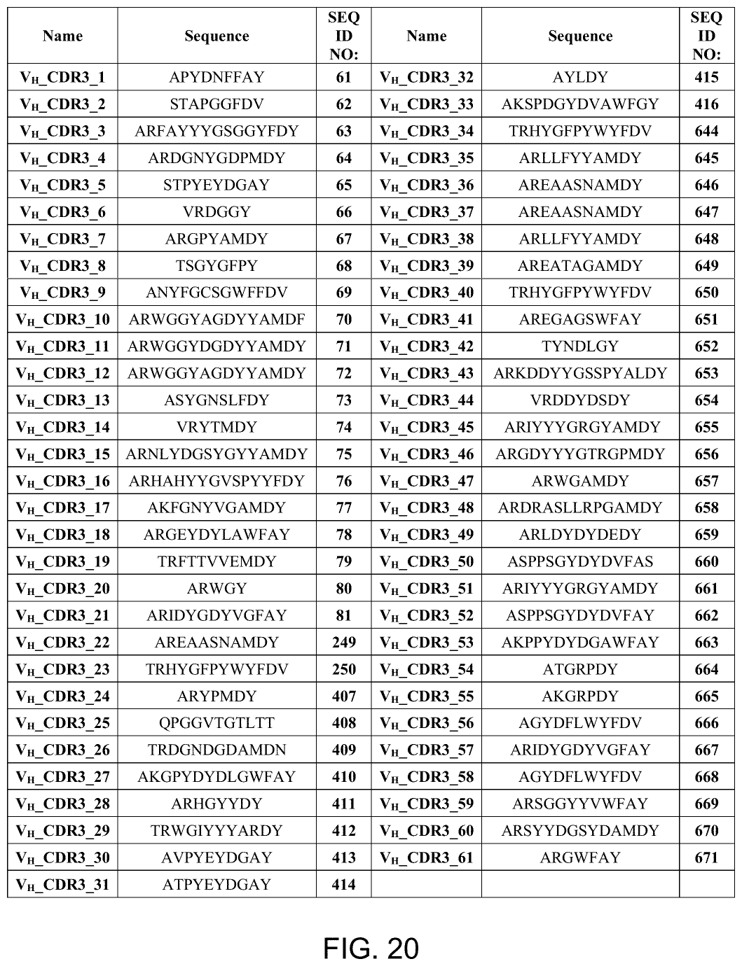

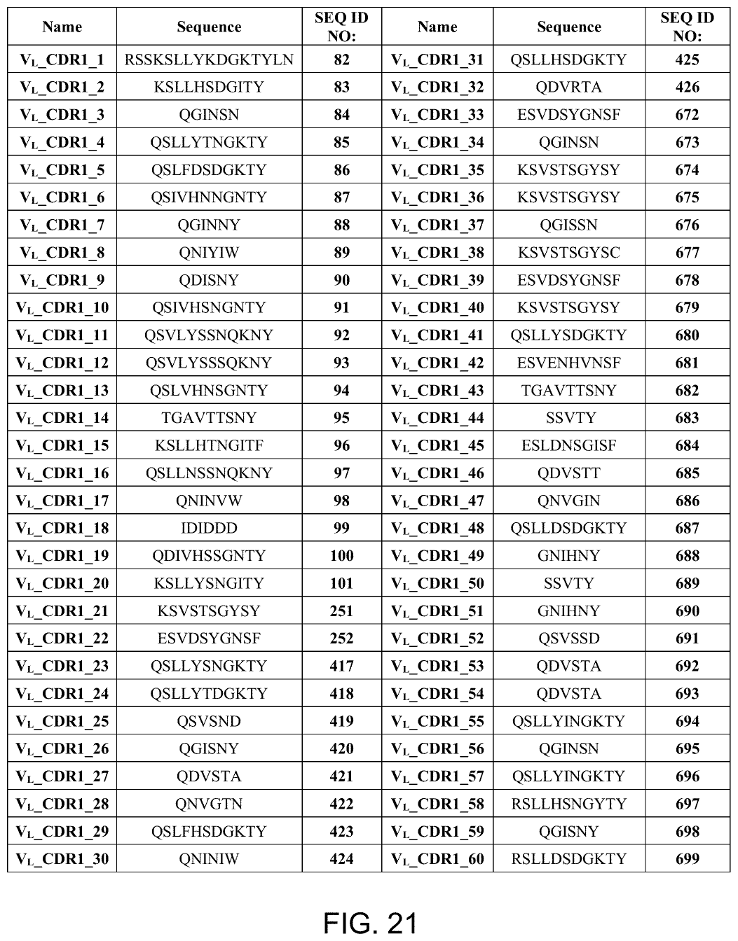

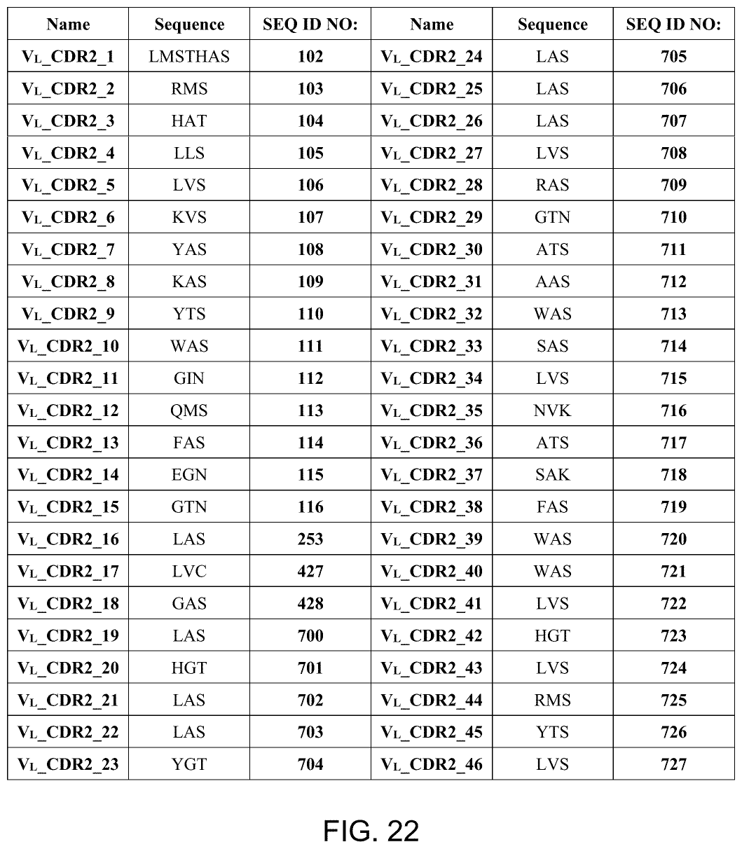

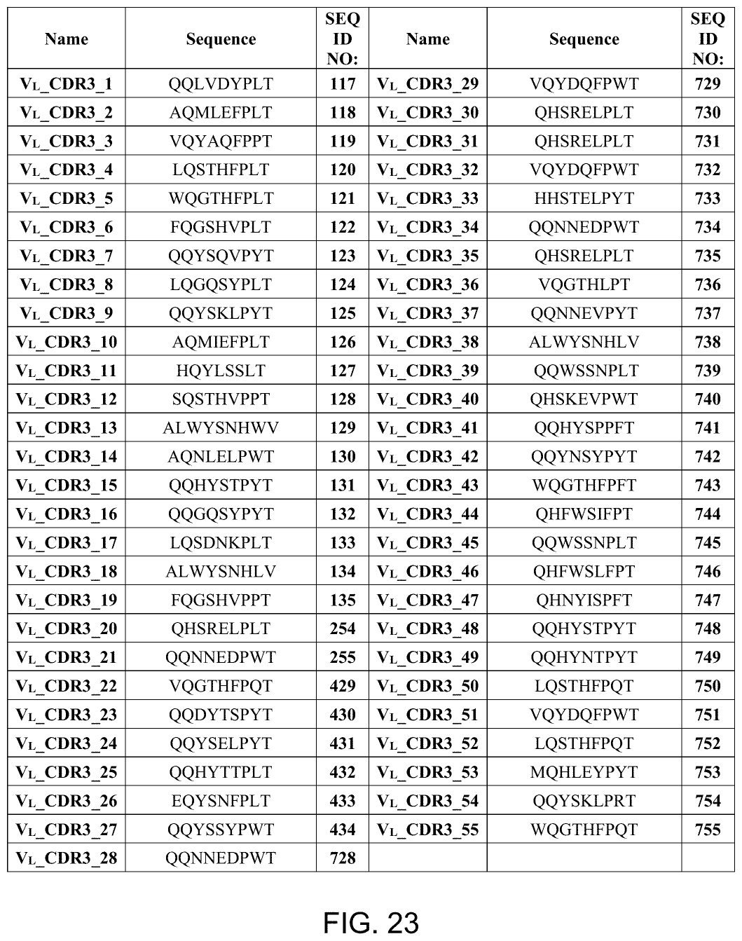

In some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23. In some embodiments, any of the anti-Gal3 antibodies or binding fragments thereof or any arrangement of any of the anti-Gal3 antibodies or binding fragments provided herein may be substituted with an antigen binding molecule that binds to Gal3.

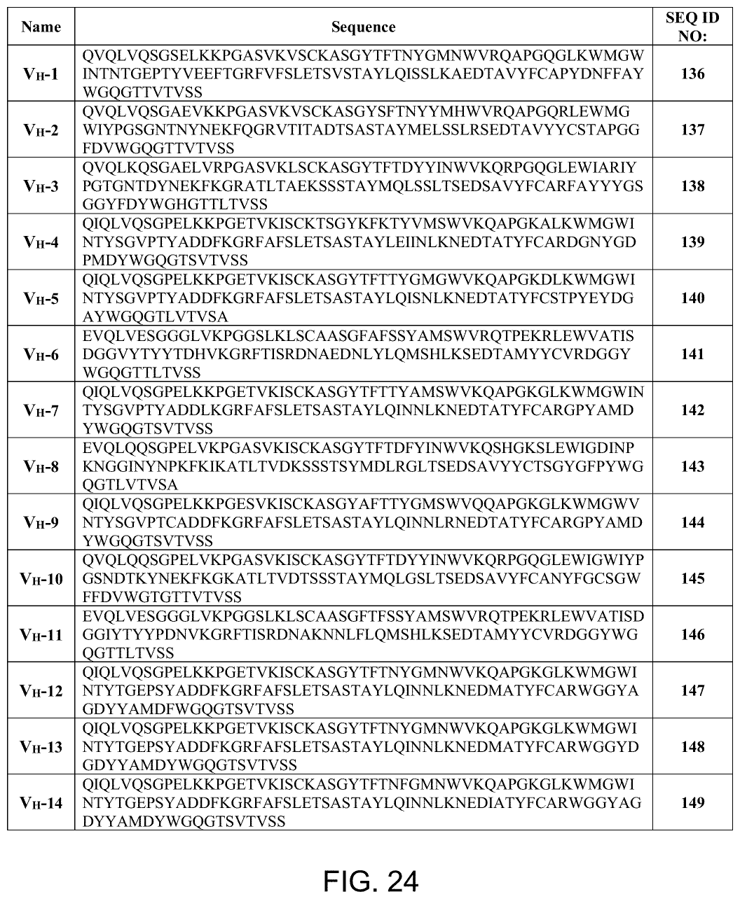

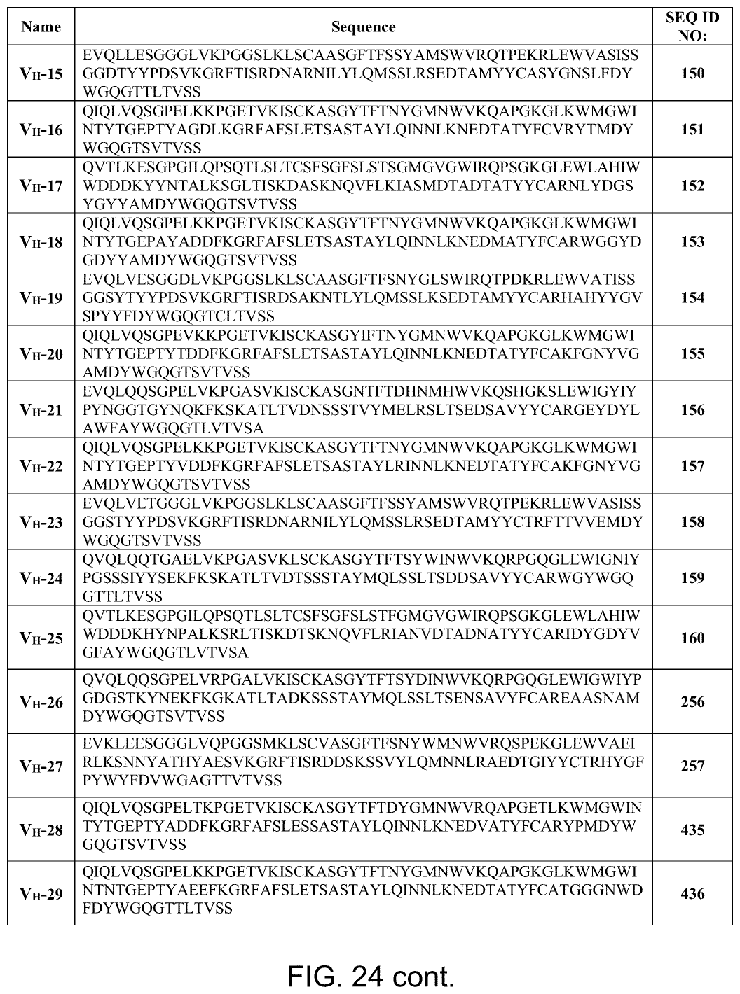

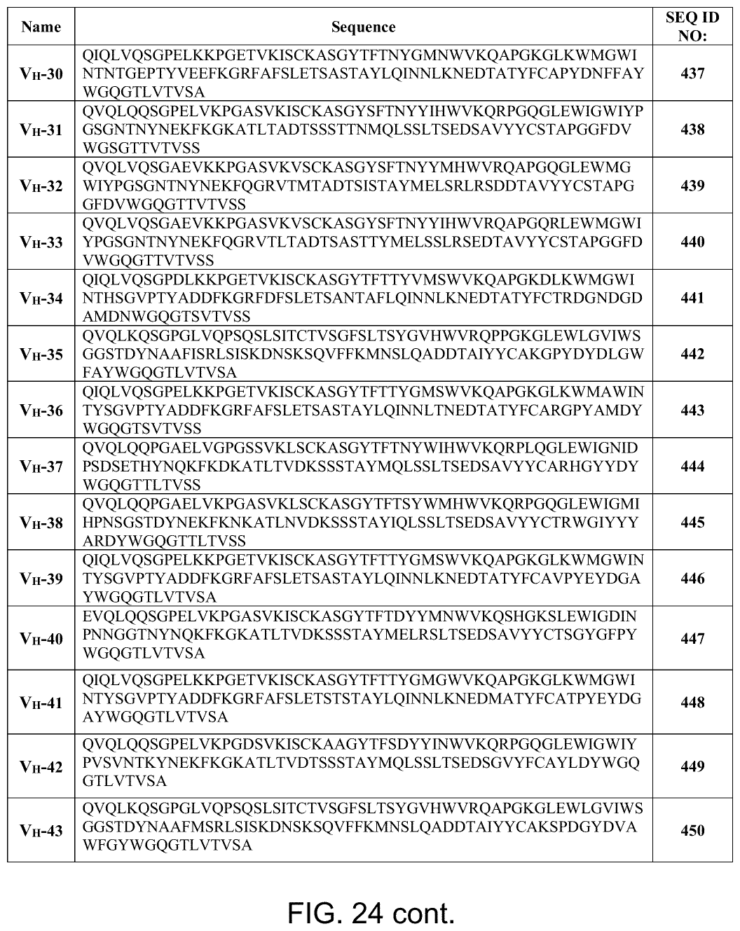

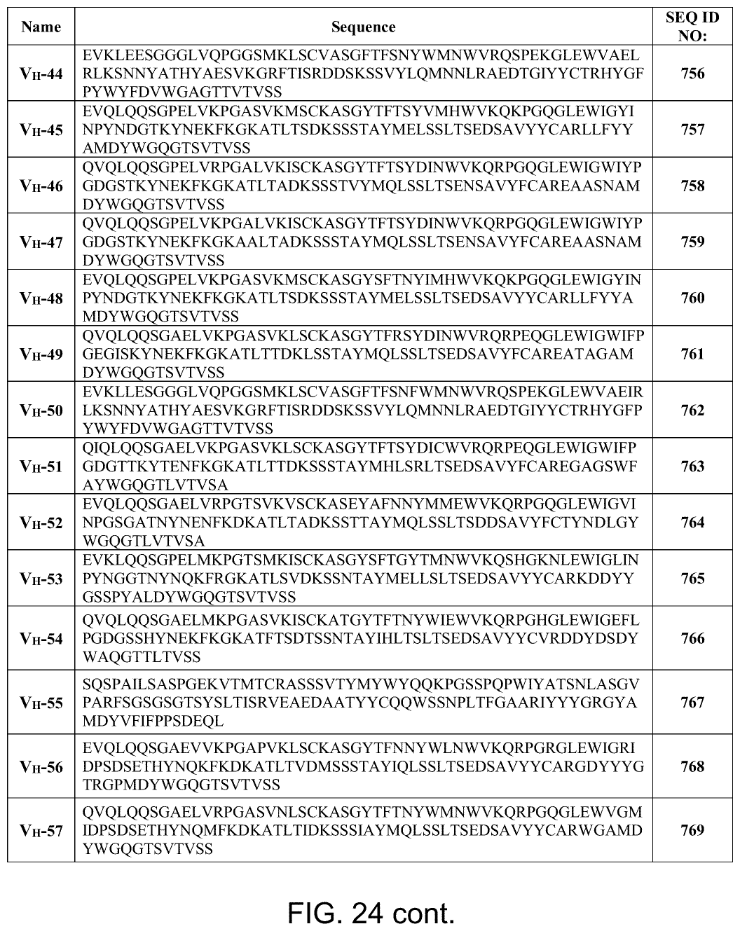

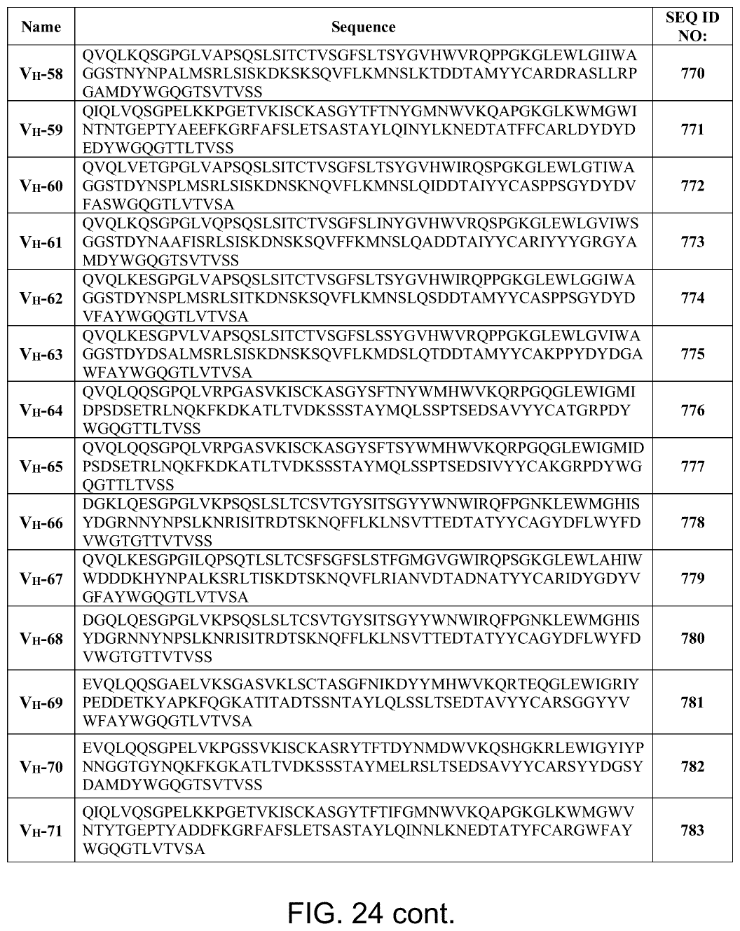

In some embodiments, the heavy chain variable region (V_{H}) comprises an amino acid sequence having at least 75%, 80%, 85%, 90%, 95%, or 100% sequence identity to any sequence according to SEQ ID NOs: 136-160, 256-257, 435-450, 756-783. In some embodiments, the heavy chain variable region is selected from the group consisting of SEQ ID NOs: 136-160, 256-257, 435-450, 756-783. In some embodiments, exemplary V_{H} are depicted in FIG. 24. In some embodiments, any of the anti-Gal3 antibodies or binding fragments thereof or any arrangement of any of the anti-Gal3 antibodies or binding fragments provided herein may be substituted with an antigen binding molecule that binds to Gal3.

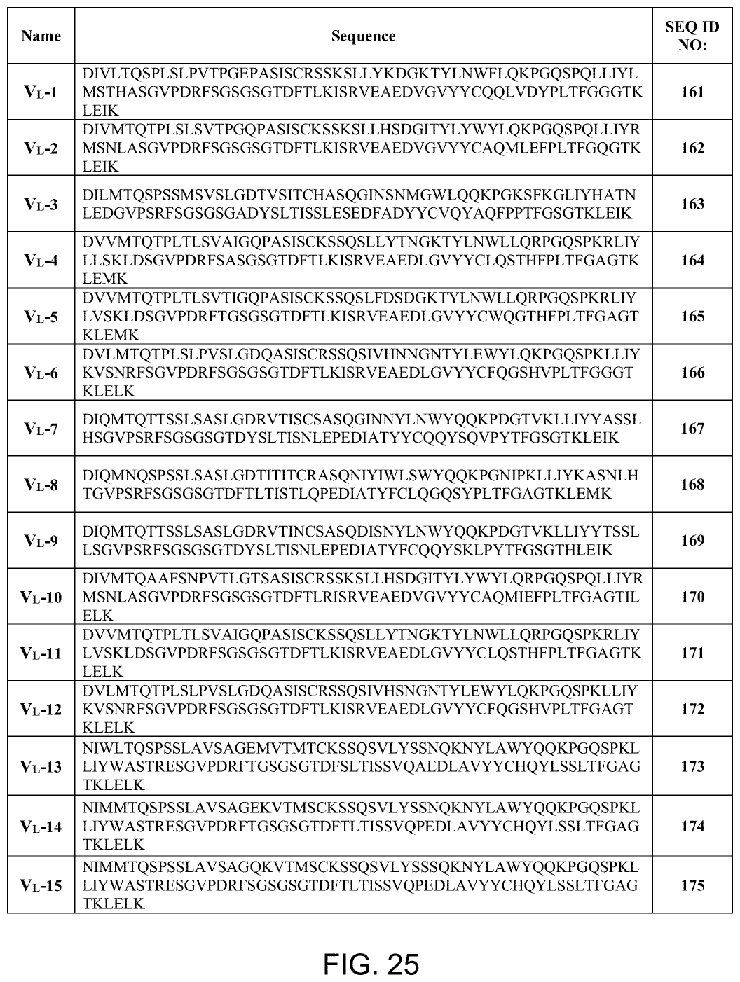

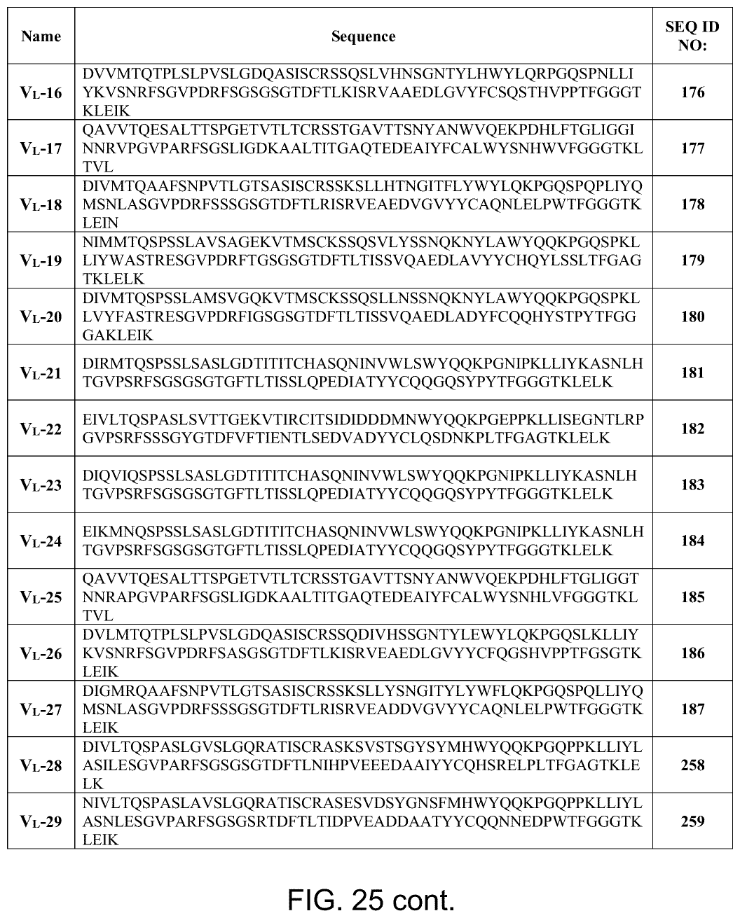

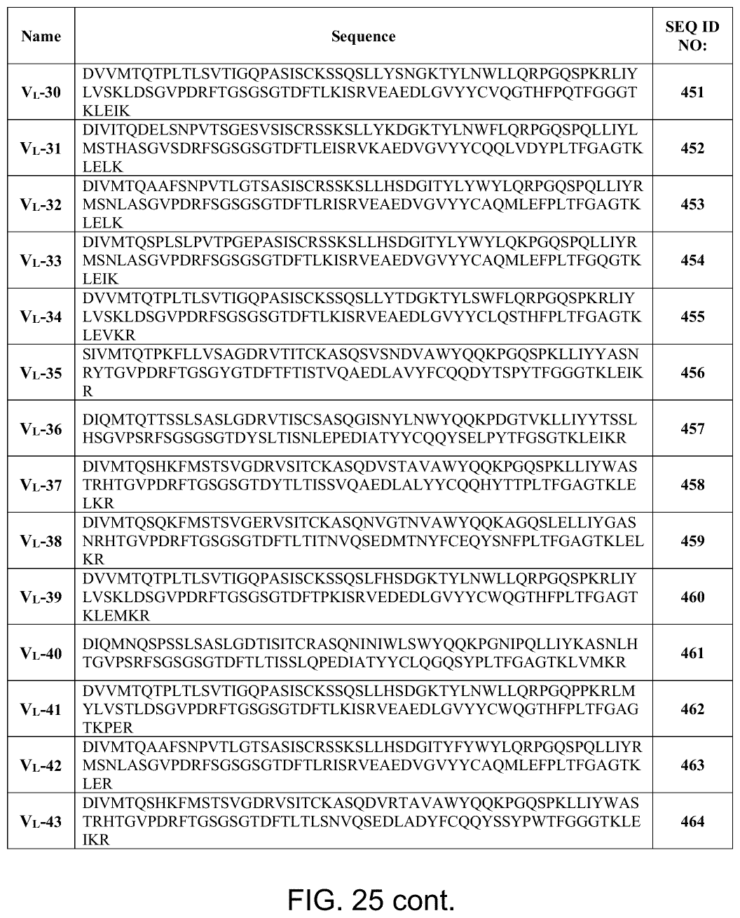

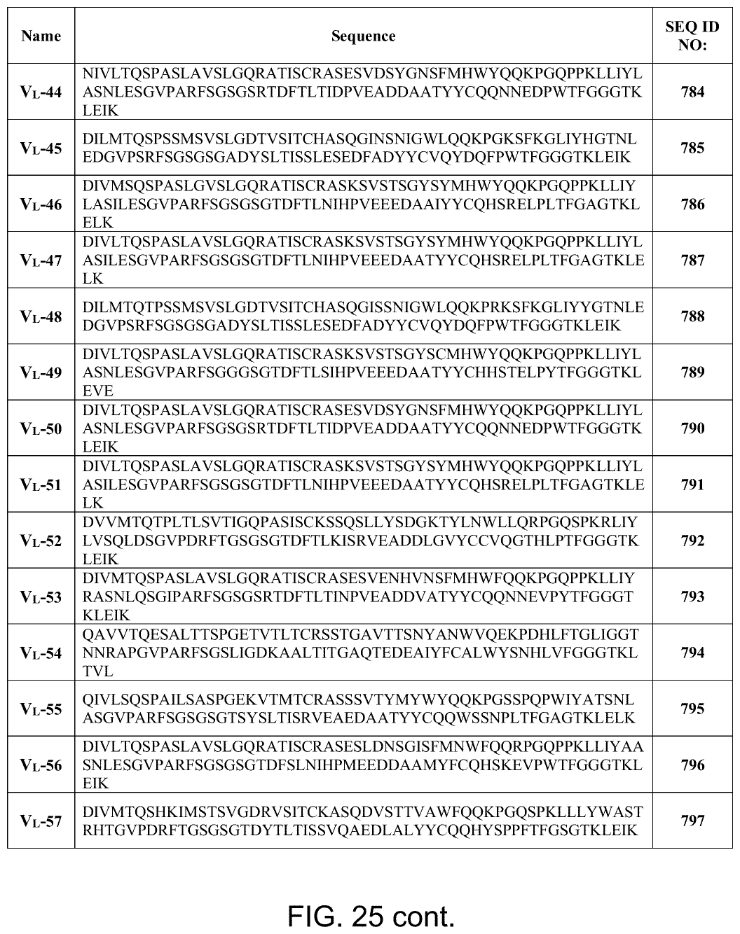

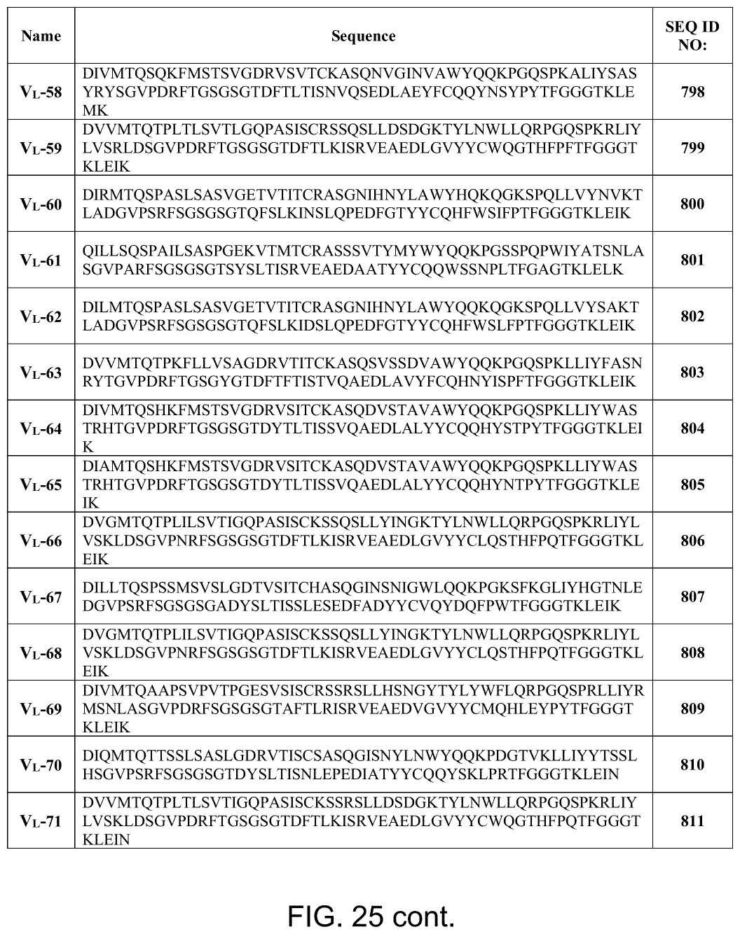

In some embodiments, the light chain variable region (V_{L}) comprises an amino acid sequence having at least 75%, 80%, 85%, 90%, 95%, or 100% sequence identity to any sequence according to SEQ ID NOs: 161-187, 258-259, 451-464, 784-811. In some embodiments, the light chain variable region is selected from the group consisting of SEQ ID NOs: 161-187, 258-259, 451-464, 784-811. In some embodiments, exemplary V_{L} are depicted in FIG. 25. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof comprises 1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161; 2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162; 3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163; 4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164; 5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, VL-CDR2, V_{L}-CDR3 within SEQ ID NO: 165; 6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166; 7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167; 8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168; 9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169; 10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170; 11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171; 12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172; 13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173; 14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175; 16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176; 17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177; 18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178; 19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179; 20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180; 21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181; 22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182; 23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183; 24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184; 25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185; 26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186; 27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187; 28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258; 29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259; 30) the V_{H}-CDR1, V_{H}-CDR2, V_{H}-CDR3 of the V_{H}-CDR1, V_{H}-CDR2, V_{H}-CDR3 within SEQ ID NO: 435 and the V_{L}-CDR1, V_{L}-CDR2, V_{L}-CDR3 of the V_{L}-CDR1, V_{L}-CDR2, V_{L}-CDR3 within SEQ ID NO: 185; 31) the V_{H}-CDR1, V_{H}-CDR2, V_{H}-CDR3 of the V_{H}-CDR1, V_{H}-CDR2, V_{H}-CDR3 within SEQ ID NO: 436 and the V_{L}-CDR1, V_{L}-CDR2, V_{L}-CDR3 of the V_{L}-CDR1, V_{L}-CDR2, V_{L}-CDR3 within SEQ ID NO: 451; 32) the V_{H}-CDR1, V_{H}-CDR2, V_{H}-CDR3 of the V_{H}-CDR1, V_{H}-CDR2, V_{H}-CDR3 within SEQ ID NO: 437 and the V_{L}-CDR1, V_{L}-CDR2, V_{L}-CDR3 of the V_{L}-CDR1, V_{L}-CDR2, V_{L}-CDR3 within SEQ ID NO: 452; 33) the V_{H}-CDR1, V_{H}-CDR2, V_{H}-CDR3 of the V_{H}-CDR1, V_{H}-CDR2, V_{H}-CDR3 within SEQ ID NO: 438 and the V_{L}-CDR1, V_{L}-CDR2, V_{L}-CDR3 of the V_{L}-CDR1, V_{L}-CDR2, V_{L}-CDR3 within SEQ ID NO: 453; 34) the V_{H}-CDR1, V_{H}-CDR2, V_{H}-CDR3 of the V_{H}-CDR1, V_{H}-CDR2, V_{H}-CDR3 within SEQ ID NO: 439 and the V_{L}-CDR1, V_{L}-CDR2, V_{L}-CDR3 of the V_{L}-CDR1, V_{L}-CDR2, V_{L}-CDR3 within SEQ ID NO: 162; 35) the V_{H}-CDR1, V_{H}-CDR2, V_{H}-CDR3 of the V_{H}-CDR1, V_{H}-CDR2, V_{H}-CDR3 within SEQ ID NO: 440 and the V_{L}-CDR1, V_{L}-CDR2, V_{L}-CDR3 of the V_{L}-CDR1, V_{L}-CDR2, V_{L}-CDR3 within SEQ ID NO: 454; 36) the V_{H}-CDR1, V_{H}-CDR2, V_{H}-CDR3 of the V_{H}-CDR1, V_{H}-CDR2, V_{H}-CDR3 within SEQ ID NO: 441 and the V_{L}-CDR1, V_{L}-CDR2, V_{L}-CDR3 of the V_{L}-CDR1, V_{L}-CDR2, V_{L}-CDR3 within SEQ ID NO: 455; 37) the V_{H}-CDR1, V_{H}-CDR2, V_{H}-CDR3 of the V_{H}-CDR1, V_{H}-CDR2, V_{H}-CDR3 within SEQ ID NO: 442 and the V_{L}-CDR1, V_{L}-CDR2, V_{L}-CDR3 of the V_{L}-CDR1, V_{L}-CDR2, V_{L}-CDR3 within SEQ ID NO: 456; 38) the V_{H}-CDR1, V_{H}-CDR2, V_{H}-CDR3 of the V_{H}-CDR1, V_{H}-CDR2, V_{H}-CDR3 within SEQ ID NO: 443 and the V_{L}-CDR1, V_{L}-CDR2, V_{L}-CDR3 of the V_{L}-CDR1, V_{L}-CDR2, V_{L}-CDR3 within SEQ ID NO: 457; 39) the V_{H}-CDR1, V_{H}-CDR2, V_{H}-CDR3 of the V_{H}-CDR1, V_{H}-CDR2, V_{H}-CDR3 within SEQ ID NO: 444 and the V_{L}-CDR1, V_{L}-CDR2, V_{L}-CDR3 of the V_{L}-CDR1, V_{L}-CDR2, V_{L}-CDR3 within SEQ ID NO: 458; 40) the V_{H}-CDR1, V_{H}-CDR2, V_{H}-CDR3 of the V_{H}-CDR1, V_{H}-CDR2, V_{H}-CDR3 within SEQ ID NO: 445 and the V_{L}-CDR1, V_{L}-CDR2, V_{L}-CDR3 of the V_{L}-CDR1, V_{L}-CDR2, V_{L}-CDR3 within SEQ ID NO: 459; 41) the V_{H}-CDR1, V_{H}-CDR2, V_{H}-CDR3 of the V_{H}-CDR1, V_{H}-CDR2, V_{H}-CDR3 within SEQ ID NO: 446 and the V_{L}-CDR1, V_{L}-CDR2, V_{L}-CDR3 of the V_{L}-CDR1, V_{L}-CDR2, V_{L}-CDR3 within SEQ ID NO: 460; 42) the V_{H}-CDR1, V_{H}-CDR2, V_{H}-CDR3 of the V_{H}-CDR1, V_{H}-CDR2, V_{H}-CDR3 within SEQ ID NO: 447 and the V_{L}-CDR1, V_{L}-CDR2, V_{L}-CDR3 of the V_{L}-CDR1, V_{L}-CDR2, V_{L}-CDR3 within SEQ ID NO: 461; 43) the V_{H}-CDR1, V_{H}-CDR2, V_{H}-CDR3 of the V_{H}-CDR1, V_{H}-CDR2, V_{H}-CDR3 within SEQ ID NO: 448 and the V_{L}-CDR1, V_{L}-CDR2, V_{L}-CDR3 of the V_{L}-CDR1, V_{L}-CDR2, V_{L}-CDR3 within SEQ ID NO: 462; 44) the V_{H}-CDR1, V_{H}-CDR2, V_{H}-CDR3 of the V_{H}-CDR1, V_{H}-CDR2, V_{H}-CDR3 within SEQ ID NO: 449 and the V_{L}-CDR1, V_{L}-CDR2, V_{L}-CDR3 of the V_{L}-CDR1, V_{L}-CDR2, V_{L}-CDR3 within SEQ ID NO: 463; 45) the V_{H}-CDR1, V_{H}-CDR2, V_{H}-CDR3 of the V_{H}-CDR1, V_{H}-CDR2, V_{H}-CDR3 within SEQ ID NO: 450 and the V_{L}-CDR1, V_{L}-CDR2, V_{L}-CDR3 of the V_{L}-CDR1, V_{L}-CDR2, V_{L}-CDR3 within SEQ ID NO: 464; 46) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 47) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 48) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 49) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 50) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 51) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 52) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 53) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 54) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 55) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 56) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 57) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 58) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 59) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 60) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 61) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 62) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 63) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 64) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 65) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 66) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 67) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 68) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 69) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 70) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 71) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 72) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 73) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

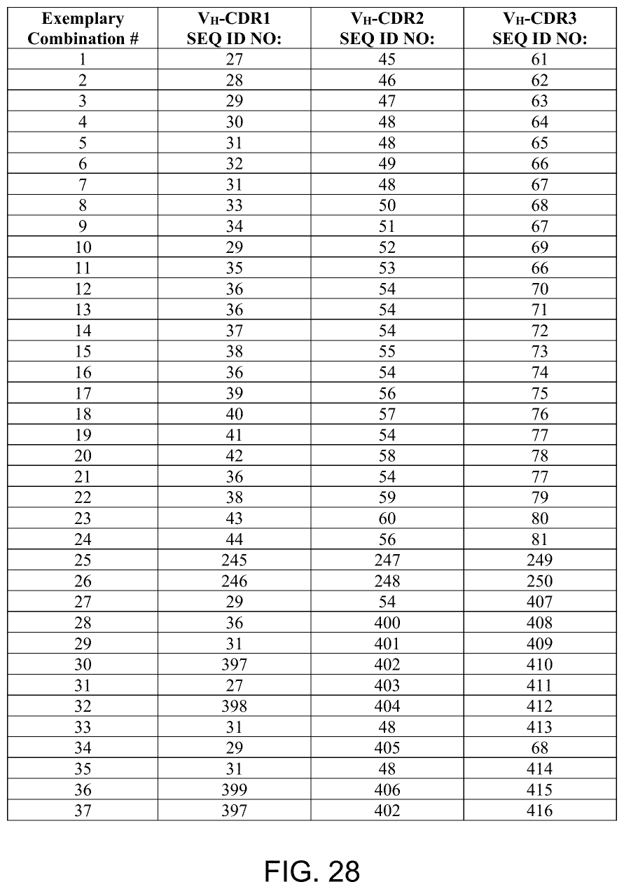

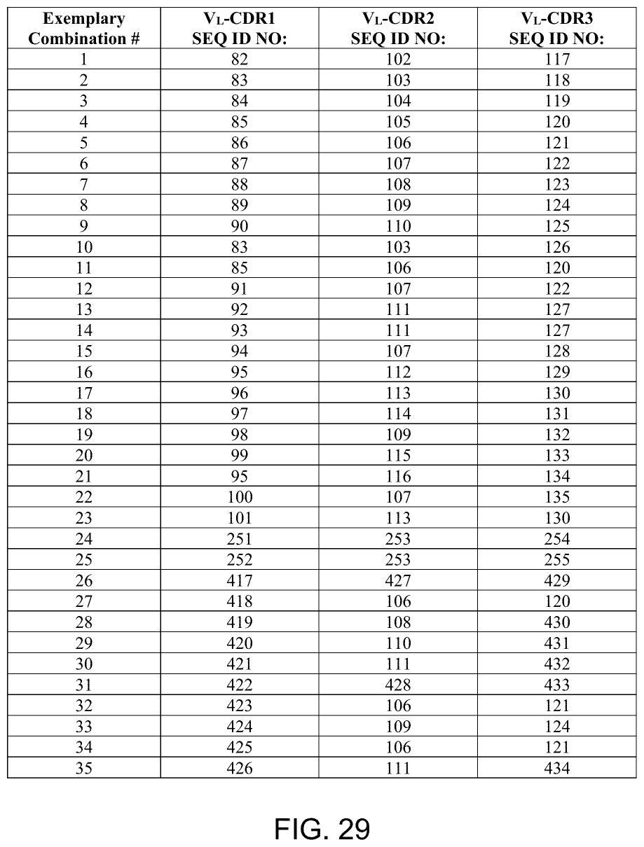

In some embodiments, exemplary combinations of heavy chain variable region CDRs are depicted in FIG. 28. In some embodiments, exemplary combinations of light chain variable region CDRs are depicted in FIG. 29. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof comprises 1) the heavy chain variable region of SEQ ID NO: 136 and the light chain variable region of SEQ ID NO: 161; 2) the heavy chain variable region of SEQ ID NO: 137 and the light chain variable region of SEQ ID NO: 162; 3) the heavy chain variable region of SEQ ID NO: 138 and the light chain variable region of SEQ ID NO: 163; 4) the heavy chain variable region of SEQ ID NO: 139 and the light chain variable region of SEQ ID NO: 164; 5) the heavy chain variable region of SEQ ID NO: 140 and the light chain variable region of SEQ ID NO: 165; 6) the heavy chain variable region of SEQ ID NO: 141 and the light chain variable region of SEQ ID NO: 166; 7) the heavy chain variable region of SEQ ID NO: 142 and the light chain variable region of SEQ ID NO: 167; 8) the heavy chain variable region of SEQ ID NO: 143 and the light chain variable region of SEQ ID NO: 168; 9) the heavy chain variable region of SEQ ID NO: 144 and the light chain variable region of SEQ ID NO: 169; 10) the heavy chain variable region of SEQ ID NO: 145 and the light chain variable region of SEQ ID NO: 170; 11) the heavy chain variable region of SEQ ID NO: 139 and the light chain variable region of SEQ ID NO: 171; 12) the heavy chain variable region of SEQ ID NO: 146 and the light chain variable region of SEQ ID NO: 172; 13) the heavy chain variable region of SEQ ID NO: 147 and the light chain variable region of SEQ ID NO: 173; 14) the heavy chain variable region of SEQ ID NO: 148 and the light chain variable region of SEQ ID NO: 174; 15) the heavy chain variable region of SEQ ID NO: 149 and the light chain variable region of SEQ ID NO: 175; 16) the heavy chain variable region of SEQ ID NO: 150 and the light chain variable region of SEQ ID NO: 176; 17) the heavy chain variable region of SEQ ID NO: 151 and the light chain variable region of SEQ ID NO: 177; 18) the heavy chain variable region of SEQ ID NO: 152 and the light chain variable region of SEQ ID NO: 178; 19) the heavy chain variable region of SEQ ID NO: 153 and the light chain variable region of SEQ ID NO: 179; 20) the heavy chain variable region of SEQ ID NO: 154 and the light chain variable region of SEQ ID NO: 180; 21) the heavy chain variable region of SEQ ID NO: 155 and the light chain variable region of SEQ ID NO: 181; 22) the heavy chain variable region of SEQ ID NO: 156 and the light chain variable region of SEQ ID NO: 182; 23) the heavy chain variable region of SEQ ID NO: 157 and the light chain variable region of SEQ ID NO: 183; 24) the heavy chain variable region of SEQ ID NO: 155 and the light chain variable region of SEQ ID NO: 184; 25) the heavy chain variable region of SEQ ID NO: 158 and the light chain variable region of SEQ ID NO: 185; 26) the heavy chain variable region of SEQ ID NO: 159 and the light chain variable region of SEQ ID NO: 186; 27) the heavy chain variable region of SEQ ID NO: 160 and the light chain variable region of SEQ ID NO: 187; 28) the heavy chain variable region of SEQ ID NO: 256 and the light chain variable region of SEQ ID NO: 258; 29) the heavy chain variable region of SEQ ID NO: 257 and the light chain variable region of SEQ ID NO: 259; 30) the heavy chain variable region of SEQ ID NO: 435 and the light chain variable region of SEQ ID NO: 185; 31) the heavy chain variable region of SEQ ID NO: 436 and the light chain variable region of SEQ ID NO: 451; 32) the heavy chain variable region of SEQ ID NO: 437 and the light chain variable region of SEQ ID NO: 452; 33) the heavy chain variable region of SEQ ID NO: 438 and the light chain variable region of SEQ ID NO: 453; 34) the heavy chain variable region of SEQ ID NO: 439 and the light chain variable region of SEQ ID NO: 162; 35) the heavy chain variable region of SEQ ID NO: 440 and the light chain variable region of SEQ ID NO: 454; 36) the heavy chain variable region of SEQ ID NO: 441 and the light chain variable region of SEQ ID NO: 455; 37) the heavy chain variable region of SEQ ID NO: 442 and the light chain variable region of SEQ ID NO: 456; 38) the heavy chain variable region of SEQ ID NO: 443 and the light chain variable region of SEQ ID NO: 457; 39) the heavy chain variable region of SEQ ID NO: 444 and the light chain variable region of SEQ ID NO: 458; 40) the heavy chain variable region of SEQ ID NO: 445 and the light chain variable region of SEQ ID NO: 459; 41) the heavy chain variable region of SEQ ID NO: 446 and the light chain variable region of SEQ ID NO: 460; 42) the heavy chain variable region of SEQ ID NO: 447 and the light chain variable region of SEQ ID NO: 461; 43) the heavy chain variable region of SEQ ID NO: 448 and the light chain variable region of SEQ ID NO: 462; 44) the heavy chain variable region of SEQ ID NO: 449 and the light chain variable region of SEQ ID NO: 463; 45) the heavy chain variable region of SEQ ID NO: 450 and the light chain variable region of SEQ ID NO: 464; 46) the heavy chain variable region of SEQ ID NO: 756 and the light chain variable region of SEQ ID NO: 784; 47) the heavy chain variable region of SEQ ID NO: 757 and the light chain variable region of SEQ ID NO: 785; 48) the heavy chain variable region of SEQ ID NO: 758 and the light chain variable region of SEQ ID NO: 786; 49) the heavy chain variable region of SEQ ID NO: 759 and the light chain variable region of SEQ ID NO: 787; 50) the heavy chain variable region of SEQ ID NO: 760 and the light chain variable region of SEQ ID NO: 788; 51) the heavy chain variable region of SEQ ID NO: 761 and the light chain variable region of SEQ ID NO: 789; 52) the heavy chain variable region of SEQ ID NO: 762 and the light chain variable region of SEQ ID NO: 790; 53) the heavy chain variable region of SEQ ID NO: 763 and the light chain variable region of SEQ ID NO: 791; 54) the heavy chain variable region of SEQ ID NO: 764 and the light chain variable region of SEQ ID NO: 792; 55) the heavy chain variable region of SEQ ID NO: 765 and the light chain variable region of SEQ ID NO: 793; 56) the heavy chain variable region of SEQ ID NO: 766 and the light chain variable region of SEQ ID NO: 794; 57) the heavy chain variable region of SEQ ID NO: 767 and the light chain variable region of SEQ ID NO: 795; 58) the heavy chain variable region of SEQ ID NO: 768 and the light chain variable region of SEQ ID NO: 796; 59) the heavy chain variable region of SEQ ID NO: 769 and the light chain variable region of SEQ ID NO: 797; 60) the heavy chain variable region of SEQ ID NO: 770 and the light chain variable region of SEQ ID NO: 798; 61) the heavy chain variable region of SEQ ID NO: 771 and the light chain variable region of SEQ ID NO: 799; 62) the heavy chain variable region of SEQ ID NO: 772 and the light chain variable region of SEQ ID NO: 800; 63) the heavy chain variable region of SEQ ID NO: 773 and the light chain variable region of SEQ ID NO: 801; 64) the heavy chain variable region of SEQ ID NO: 774 and the light chain variable region of SEQ ID NO: 802; 65) the heavy chain variable region of SEQ ID NO: 775 and the light chain variable region of SEQ ID NO: 803; 66) the heavy chain variable region of SEQ ID NO: 776 and the light chain variable region of SEQ ID NO: 804; 67) the heavy chain variable region of SEQ ID NO: 777 and the light chain variable region of SEQ ID NO: 805; 68) the heavy chain variable region of SEQ ID NO: 778 and the light chain variable region of SEQ ID NO: 806; 69) the heavy chain variable region of SEQ ID NO: 779 and the light chain variable region of SEQ ID NO: 807; 70) the heavy chain variable region of SEQ ID NO: 780 and the light chain variable region of SEQ ID NO: 808; 71) the heavy chain variable region of SEQ ID NO: 781 and the light chain variable region of SEQ ID NO: 809; 72) the heavy chain variable region of SEQ ID NO: 782 and the light chain variable region of SEQ ID NO: 810; or 73) the heavy chain variable region of SEQ ID NO: 783 and the light chain variable region of SEQ ID NO: 811.

In some embodiments, any of the anti-Gal3 antibodies or binding fragments thereof or any arrangement of any of the anti-Gal3 antibodies or binding fragments provided herein may be substituted with an antigen binding molecule that binds to Gal3.

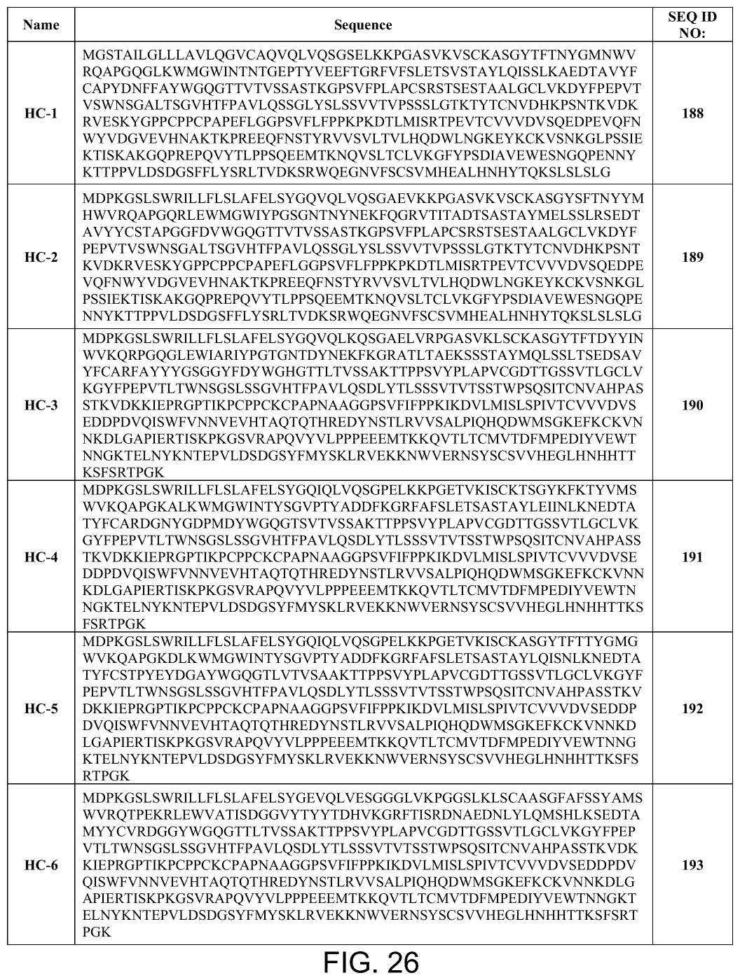

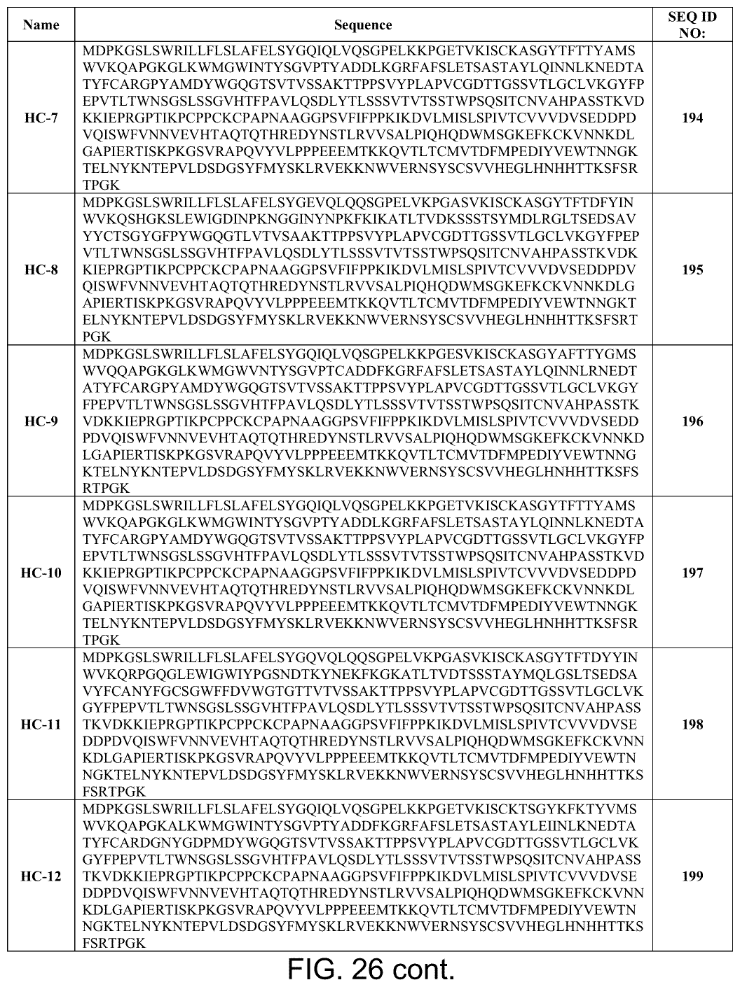

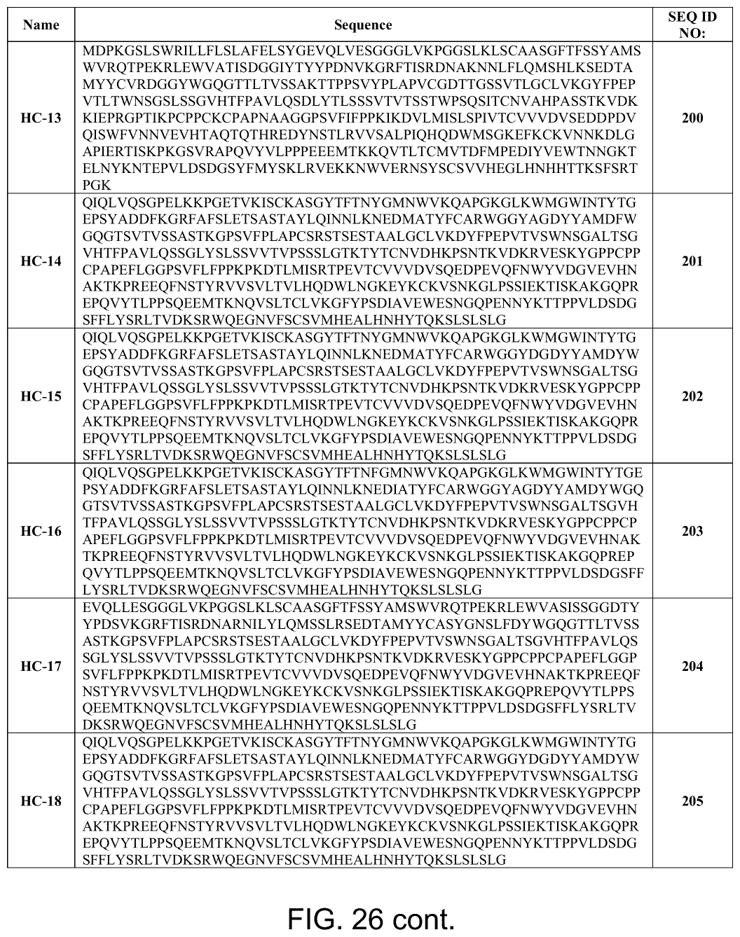

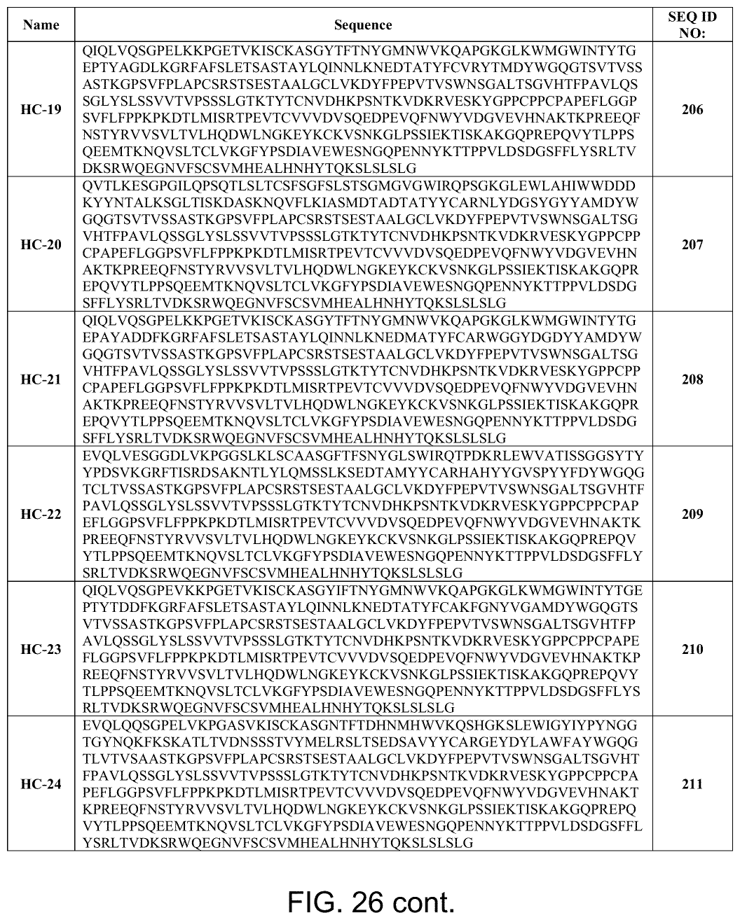

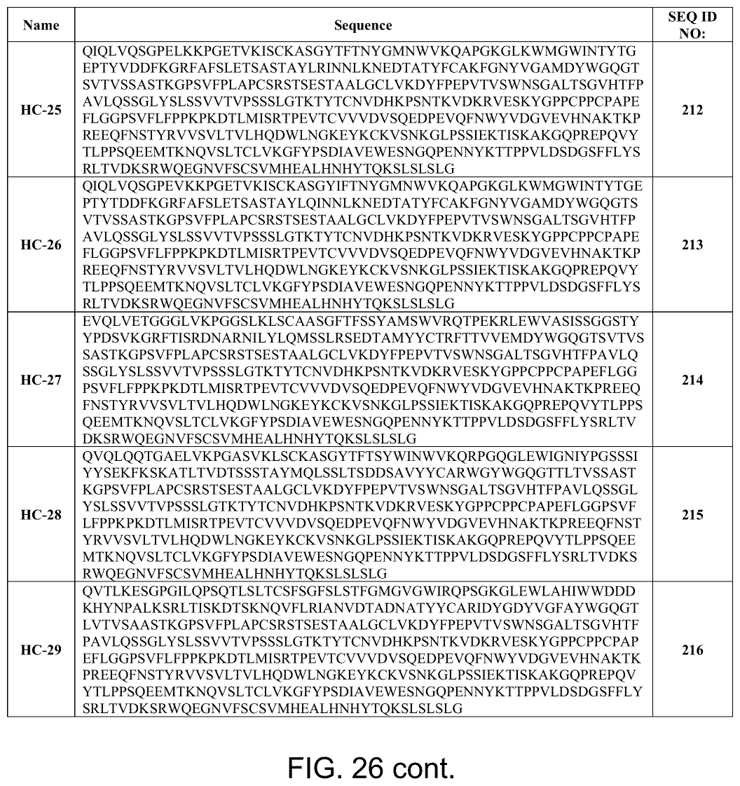

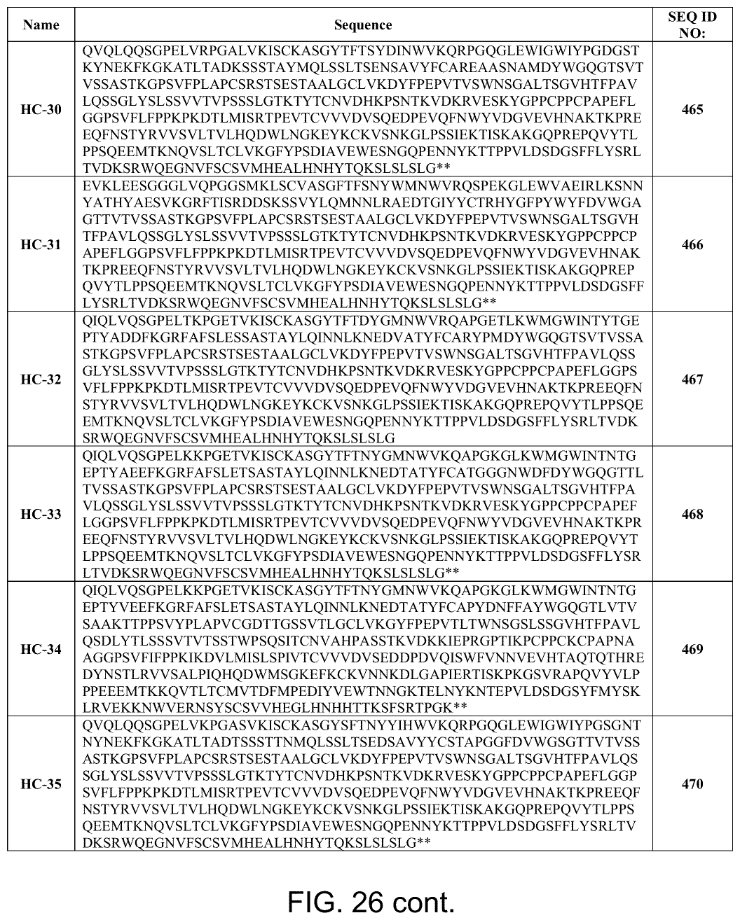

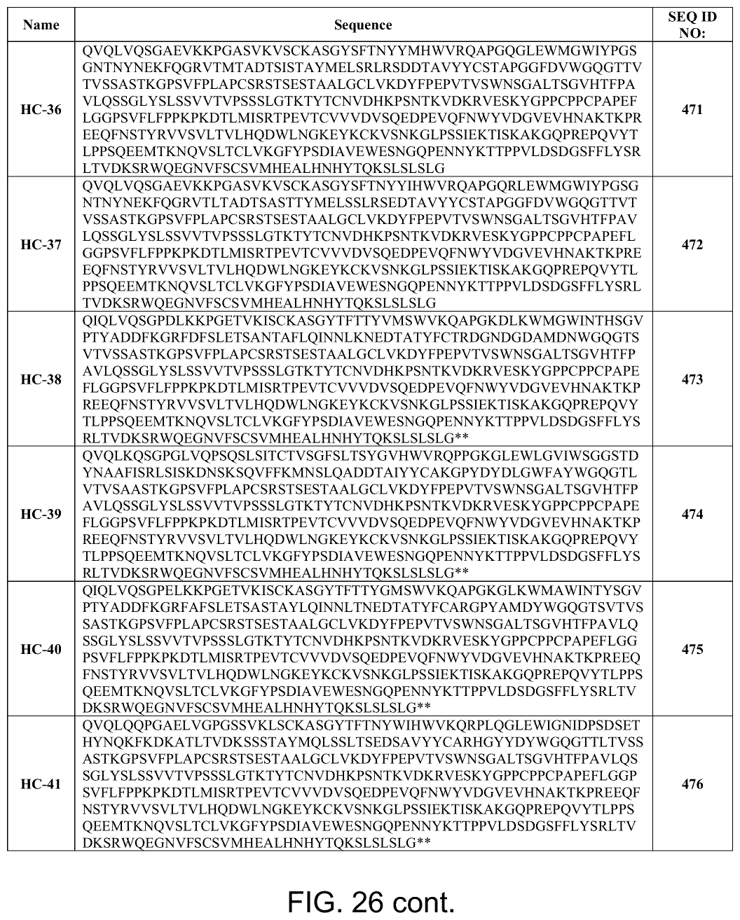

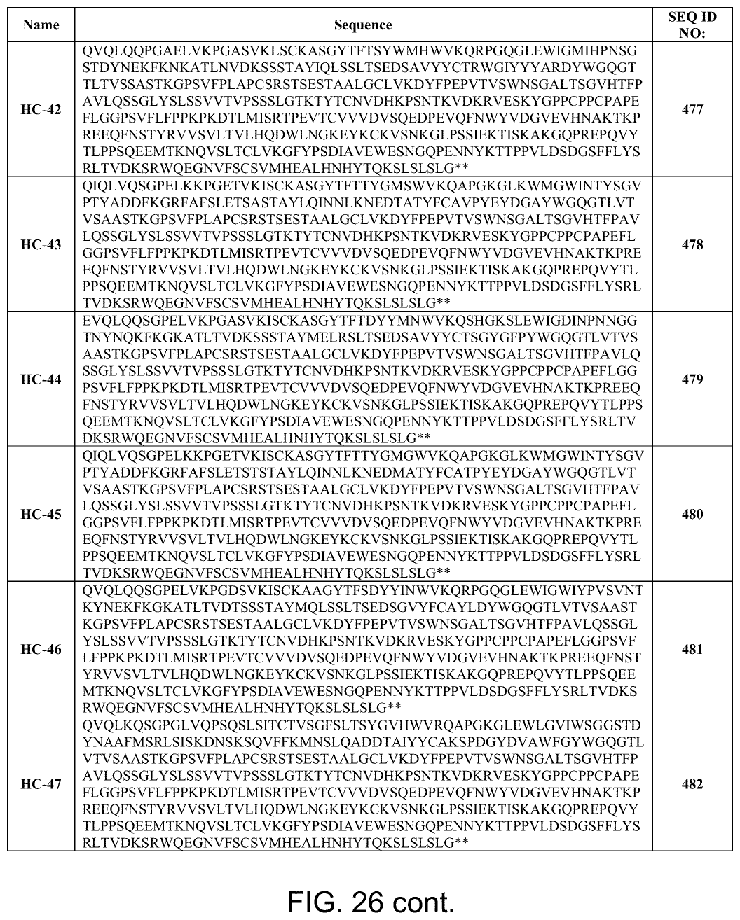

In some embodiments, the anti-Gal3 antibody or binding fragment thereof comprises the heavy chain (HC) sequence of any one of SEQ ID NOs: 188-216, 465-482. In some embodiments, exemplary HC sequences are depicted in FIG. 26. In some embodiments, any of the anti-Gal3 antibodies or binding fragments thereof or any arrangement of any of the anti-Gal3 antibodies or binding fragments provided herein may be substituted with an antigen binding molecule that binds to Gal3.

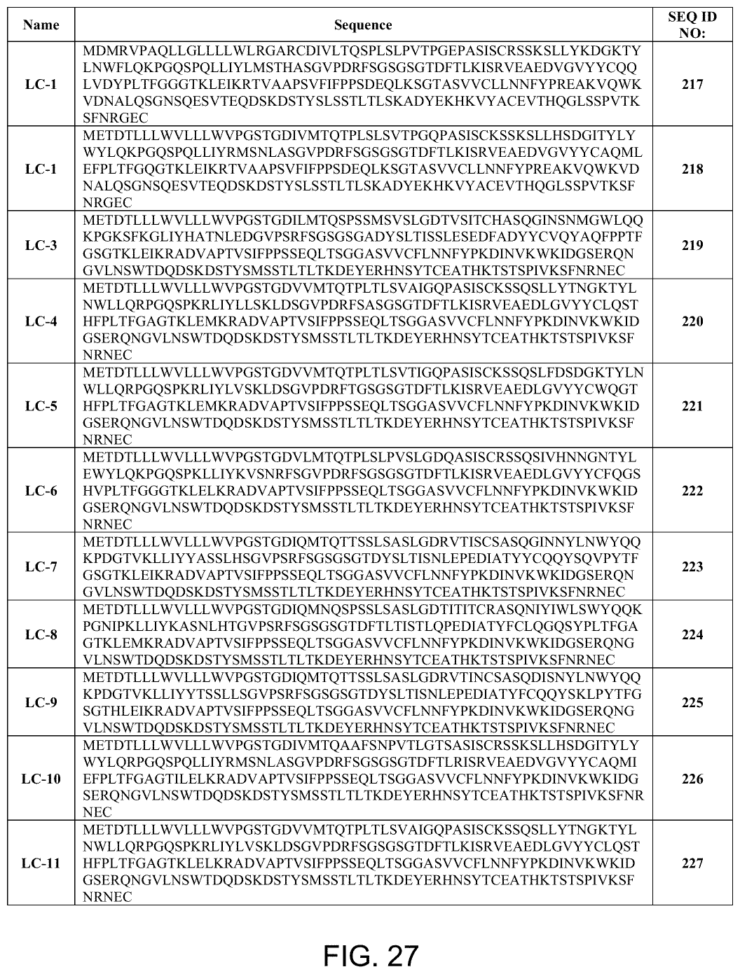

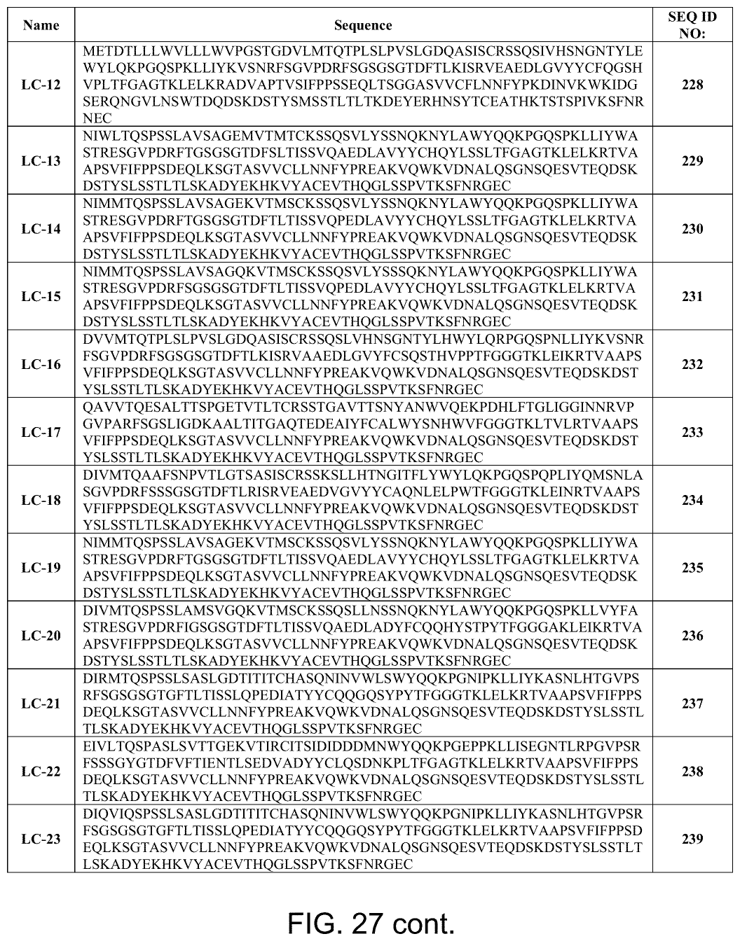

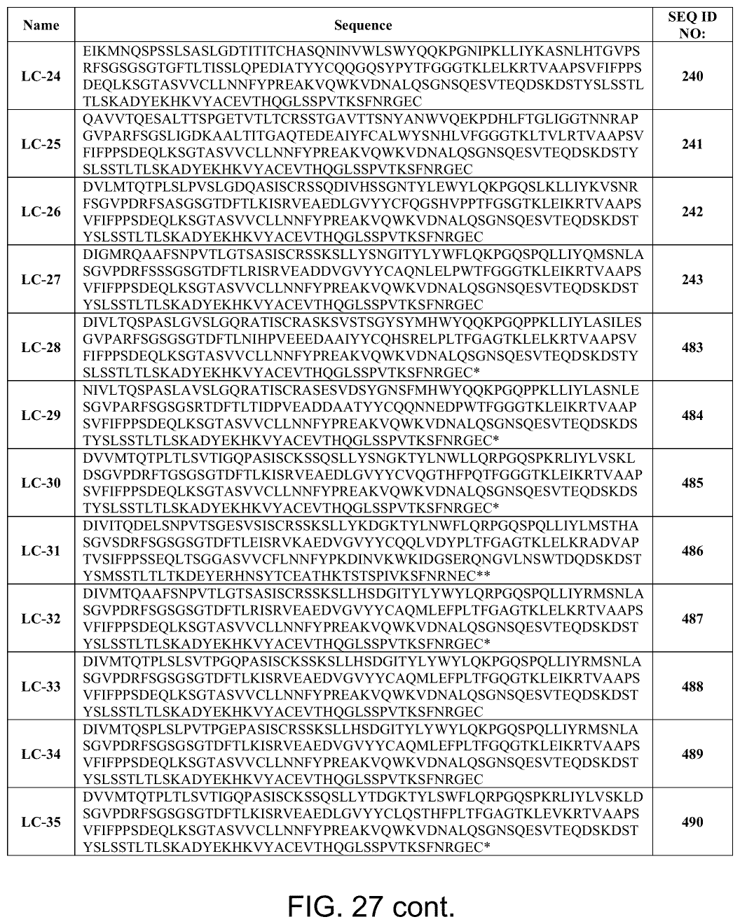

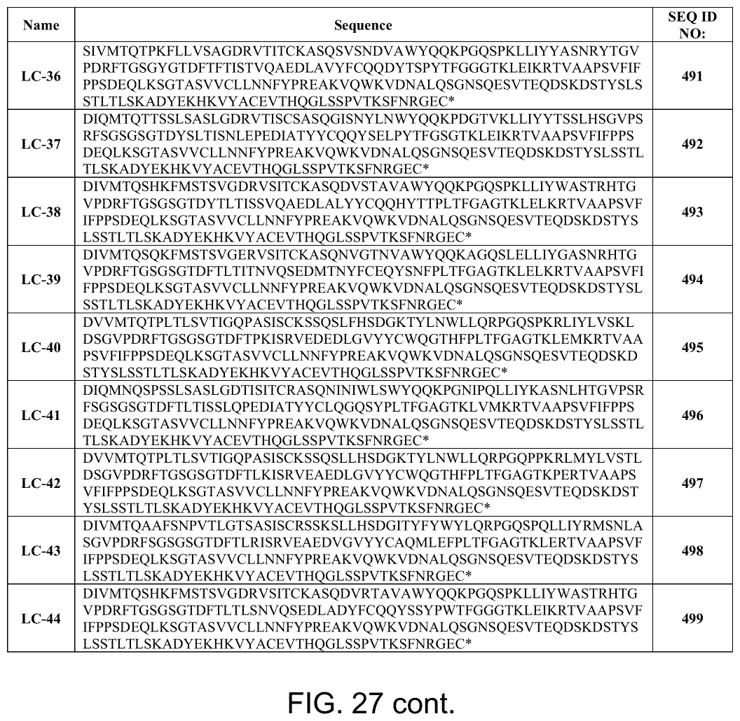

In some embodiments, the anti-Gal3 antibody or binding fragment thereof comprises the light chain (LC) sequence of any one of SEQ ID NOs: 217-243, 483-499. In some embodiments, exemplary LC sequences are depicted in FIG. 27. In some embodiments, any of the anti-Gal3 antibodies or binding fragments thereof or any arrangement of any of the anti-Gal3 antibodies or binding fragments provided herein may be substituted with an antigen binding molecule that binds to Gal3.

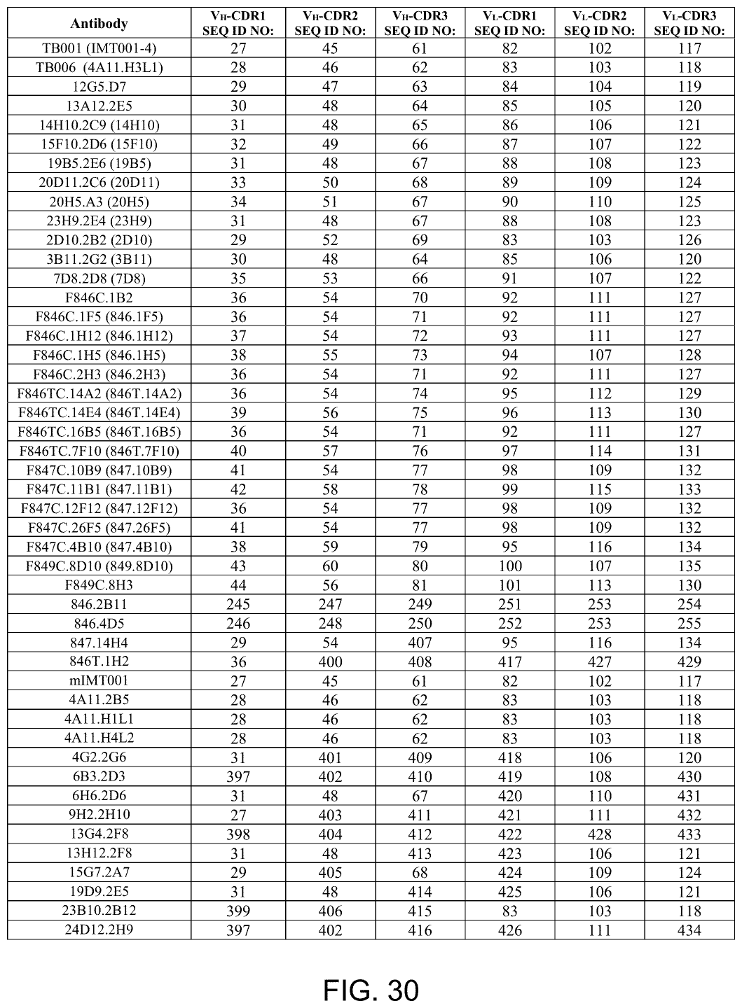

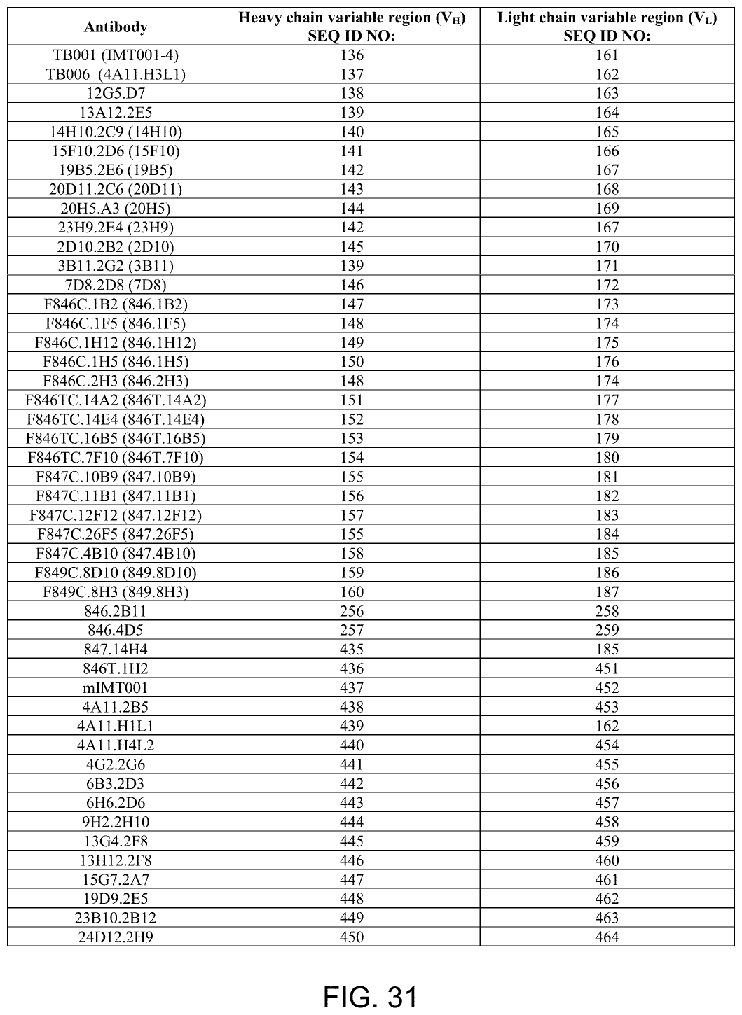

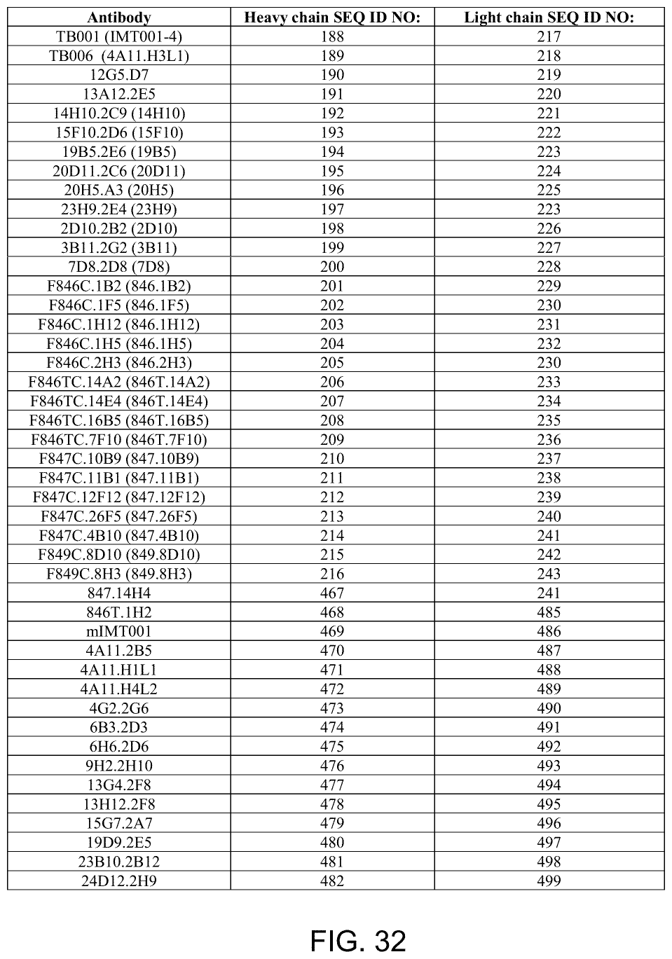

In some embodiments, the anti-Gal3 antibody or binding fragment thereof is selected from the group consisting of TB001 (IMT001), TB006 (4A11.H3L1), 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32. In some embodiments, any of the anti-Gal3 antibodies or binding fragments thereof or any arrangement of any of the anti-Gal3 antibodies or binding fragments provided herein may be substituted with an antigen binding molecule that binds to Gal3.

Disclosed herein are anti-Gal3 antibodies or binding fragments thereof. In some embodiments, the anti-Gal3 antibody or binding fragment thereof comprises (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 36-44, 588-615, the V_{H}-CDR2 comprises an amino acid sequence selected from SEQ ID NOs: 54-60, 616-643, the V_{H}-CDR3 comprises an amino acid sequence selected from SEQ ID NOs: 70-81, 644-671, the V_{L}-CDR1 comprises an amino acid sequence selected from SEQ ID NOs: 92-101, 672-699, the V_{L}-CDR2 comprises an amino acid sequence selected from SEQ ID NOs: 111-116, 700-727, and the V_{L}-CDR3 comprises an amino acid sequence selected from SEQ ID NOs: 127-135, 728-755. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the heavy chain variable region (V_{H}) comprises an amino acid sequence having at least 75%, 80%, 85%, 90%, 95%, or 100% sequence identity to any sequence according to SEQ ID NOs: 147-160, 756-783. In some embodiments, the heavy chain variable region is selected from the group consisting of SEQ ID NOs: 147-160, 756-783. In some embodiments, exemplary V_{H} are depicted in FIG. 24. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the light chain variable region (V_{L}) comprises an amino acid sequence having at least 75%, 80%, 85%, 90%, 95%, or 100% sequence identity to any sequence according to SEQ ID NOs: 173-187, 784-811. In some embodiments, the light chain variable region is selected from the group consisting of SEQ ID NOs: 173-187, 784-811. In some embodiments, exemplary VL are depicted in FIG. 25. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof comprises 1) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173; 2) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 3) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175; 4) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176; 5) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177; 6) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, VL-CDR3 of the V_{L}-CDR1, V_{L}-CDR2, V_{L}-CDR3 within SEQ ID NO: 178; 7) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179; 8) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180; 9) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181; 10) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182; 11) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183; 12) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184; 13) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185; 14) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186; 15) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187; 16) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 17) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 18) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 19) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 20) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 21) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 22) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 23) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 24) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 25) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 26) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 27) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 28) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 29) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 30) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 31) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 32) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 33) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 34) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 35) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 36) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 37) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 38) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 39) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 40) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 41) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 42) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 43) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811. In some embodiments, exemplary combinations of heavy chain variable region CDRs are depicted in FIG. 28. In some embodiments, exemplary combinations of light chain variable region CDRs are depicted in FIG. 29. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof comprises 1) the heavy chain variable region of SEQ ID NO: 147 and the light chain variable region of SEQ ID NO: 173; 2) the heavy chain variable region of SEQ ID NO: 148 and the light chain variable region of SEQ ID NO: 174; 3) the heavy chain variable region of SEQ ID NO: 149 and the light chain variable region of SEQ ID NO: 175; 4) the heavy chain variable region of SEQ ID NO: 150 and the light chain variable region of SEQ ID NO: 176; 5) the heavy chain variable region of SEQ ID NO: 151 and the light chain variable region of SEQ ID NO: 177; 6) the heavy chain variable region of SEQ ID NO: 152 and the light chain variable region of SEQ ID NO: 178; 7) the heavy chain variable region of SEQ ID NO: 153 and the light chain variable region of SEQ ID NO: 179; 8) the heavy chain variable region of SEQ ID NO: 154 and the light chain variable region of SEQ ID NO: 180; 9) the heavy chain variable region of SEQ ID NO: 155 and the light chain variable region of SEQ ID NO: 181; 10) the heavy chain variable region of SEQ ID NO: 156 and the light chain variable region of SEQ ID NO: 182; 11) the heavy chain variable region of SEQ ID NO: 157 and the light chain variable region of SEQ ID NO: 183; 12) the heavy chain variable region of SEQ ID NO: 155 and the light chain variable region of SEQ ID NO: 184; 13) the heavy chain variable region of SEQ ID NO: 158 and the light chain variable region of SEQ ID NO: 185; 14) the heavy chain variable region of SEQ ID NO: 159 and the light chain variable region of SEQ ID NO: 186; 15) the heavy chain variable region of SEQ ID NO: 160 and the light chain variable region of SEQ ID NO: 187; 16) the heavy chain variable region of SEQ ID NO: 756 and the light chain variable region of SEQ ID NO: 784; 17) the heavy chain variable region of SEQ ID NO: 757 and the light chain variable region of SEQ ID NO: 785; 18) the heavy chain variable region of SEQ ID NO: 758 and the light chain variable region of SEQ ID NO: 786; 19) the heavy chain variable region of SEQ ID NO: 759 and the light chain variable region of SEQ ID NO: 787; 20) the heavy chain variable region of SEQ ID NO: 760 and the light chain variable region of SEQ ID NO: 788; 21) the heavy chain variable region of SEQ ID NO: 761 and the light chain variable region of SEQ ID NO: 789; 22) the heavy chain variable region of SEQ ID NO: 762 and the light chain variable region of SEQ ID NO: 790; 23) the heavy chain variable region of SEQ ID NO: 763 and the light chain variable region of SEQ ID NO: 791; 24) the heavy chain variable region of SEQ ID NO: 764 and the light chain variable region of SEQ ID NO: 792; 25) the heavy chain variable region of SEQ ID NO: 765 and the light chain variable region of SEQ ID NO: 793; 26) the heavy chain variable region of SEQ ID NO: 766 and the light chain variable region of SEQ ID NO: 794; 27) the heavy chain variable region of SEQ ID NO: 767 and the light chain variable region of SEQ ID NO: 795; 28) the heavy chain variable region of SEQ ID NO: 768 and the light chain variable region of SEQ ID NO: 796; 29) the heavy chain variable region of SEQ ID NO: 769 and the light chain variable region of SEQ ID NO: 797; 30) the heavy chain variable region of SEQ ID NO: 770 and the light chain variable region of SEQ ID NO: 798; 31) the heavy chain variable region of SEQ ID NO: 771 and the light chain variable region of SEQ ID NO: 799; 32) the heavy chain variable region of SEQ ID NO: 772 and the light chain variable region of SEQ ID NO: 800; 33) the heavy chain variable region of SEQ ID NO: 773 and the light chain variable region of SEQ ID NO: 801; 34) the heavy chain variable region of SEQ ID NO: 774 and the light chain variable region of SEQ ID NO: 802; 35) the heavy chain variable region of SEQ ID NO: 775 and the light chain variable region of SEQ ID NO: 803; 36) the heavy chain variable region of SEQ ID NO: 776 and the light chain variable region of SEQ ID NO: 804; 37) the heavy chain variable region of SEQ ID NO: 777 and the light chain variable region of SEQ ID NO: 805; 38) the heavy chain variable region of SEQ ID NO: 778 and the light chain variable region of SEQ ID NO: 806; 39) the heavy chain variable region of SEQ ID NO: 779 and the light chain variable region of SEQ ID NO: 807; 40) the heavy chain variable region of SEQ ID NO: 780 and the light chain variable region of SEQ ID NO: 808; 41) the heavy chain variable region of SEQ ID NO: 781 and the light chain variable region of SEQ ID NO: 809; 42) the heavy chain variable region of SEQ ID NO: 782 and the light chain variable region of SEQ ID NO: 810; or 43) the heavy chain variable region of SEQ ID NO: 783 and the light chain variable region of SEQ ID NO: 811. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof comprises the heavy chain (HC) sequence of any one of SEQ ID NOs: 201-216. In some embodiments, exemplary HC sequences are depicted in FIG. 26. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof comprises the light chain (LC) sequence of any one of SEQ ID NOs: 229-243. In some embodiments, exemplary LC sequences are depicted in FIG. 27. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof is selected from at least one of the group consisting of F846C.1B2, F846C.1F5, F846C.1H12, F846C.2H3, F846TC.14E4, F846TC.16B5, F846TC.7F10, F849C.8D10, 846.4D5, or a binding fragment thereof. In some embodiments, the anti-Gal3 antibody or binding fragment thereof is selected from the group consisting of F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, and/or F847C.21H6, or a binding fragment thereof. In some embodiments, the anti-Gal3 antibody or binding fragment thereof disrupts an interaction between Gal3 and an antibody selected from 846.4D5, 15F10.2D6, F846C.1B2, and F846C.1H12. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof binds to one or more peptides of SEQ ID NOs: 3-26. In some embodiments, any of the anti-Gal3 antibodies or binding fragments thereof or any arrangement of any of the anti-Gal3 antibodies or binding fragments provided herein may be substituted with an antigen binding molecule that binds to Gal3.

In some embodiments, the anti-Gal3 antibody or binding fragment thereof belongs to bin 3, 8, 17, or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof competes with an antibody that belongs to bins 3, 8, 17 or 24 for binding to Gal3.

In some instances, the anti-Gal3 antibody or binding fragment thereof comprises a humanized antibody or binding fragment thereof. In other instances, the anti-Gal3 antibody or binding fragment thereof comprises a chimeric antibody or binding fragment thereof. In some cases, the anti-Gal3 antibody comprises a full-length antibody or a binding fragment thereof. In some cases, the anti-Gal3 antibody or binding fragment thereof comprises a bispecific antibody or a binding fragment thereof. In some cases, the anti-Gal3 antibody or binding fragment thereof comprises a monovalent Fab′, a divalent Fab2, a single-chain variable fragment (scFv), a diabody, a minibody, a nanobody, a single-domain antibody (sdAb), or a camelid antibody or binding fragment thereof.

Disclosed in some embodiments are methods of using any one of the anti-Gal3 antibodies, binding fragments thereof, or antigen binding molecules disclosed herein for the treatment of a disease or disorder in a subject. In some embodiments, the methods include administering any one of the anti-Gal3 antibodies, binding fragments thereof, or antigen binding molecules disclosed herein to a subject having, suspected of having, or at risk of developing a disease or disorder as described herein.

In some embodiments, any of the embodiments and/or any of the anti-Gal3 antibodies or binding fragments thereof disclosed herein may be used for any of the applications, methods, and uses provided herein.

Some embodiments provided herein relate to anti-Gal3 antibodies or binding fragments thereof. In some embodiments, the anti-Gal3 antibodies or binding fragments thereof bind to the N-terminal domain of Gal3, the N-terminus of Gal3, or the tandem repeat domain (TRD) of Gal3.

Some embodiments provided herein relate to anti-Gal3 antibodies or binding fragments thereof that disrupt the interaction between Gal3 and a protein associated with proteopathies or neurological disease. In some embodiments are methods and uses of the anti-Gal3 antibodies and binding fragments thereof disclosed herein for the treatment of proteopathies and/or neurological disease.

Some embodiments provided herein relate to anti-Gal3 antibodies or binding fragments thereof that are able to cross the blood-brain barrier. In some embodiments, the blood-brain barrier is of a subject that has a neurological disease. In some embodiments, the anti-Gal3 antibodies or binding fragments thereof are multi-specific antibodies in order to increase the permeability of another antibody across the blood-brain barrier. In some embodiments, the anti-Gal3 antibodies or binding fragments thereof are conjugated to a payload in order to increase the permeability of the payload across the blood-brain barrier.

Some embodiments provided herein relate to anti-Gal3 antibodies or binding fragments thereof that disrupt the interaction between Gal3 and a cell surface marker or a tumor cell surface marker. In some embodiments are methods and uses of the anti-Gal3 antibodies and binding fragments thereof disclosed herein for the treatment of diseases associated with the cell surface marker or tumor cell surface marker. In some embodiments, the disease is a cancer, fibrosis, or immune-related disorder.

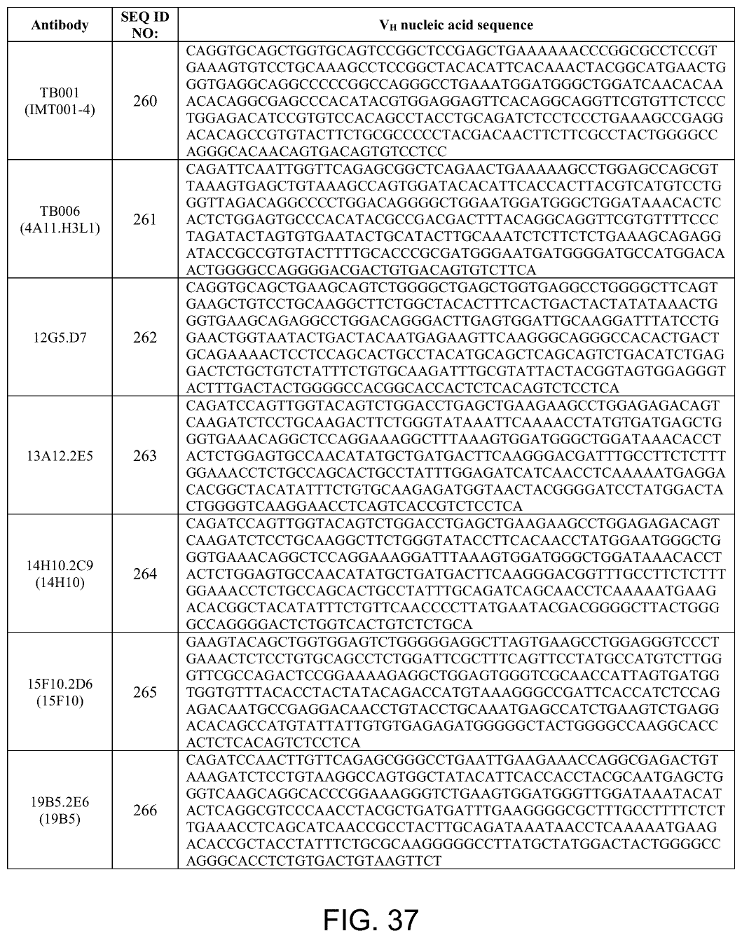

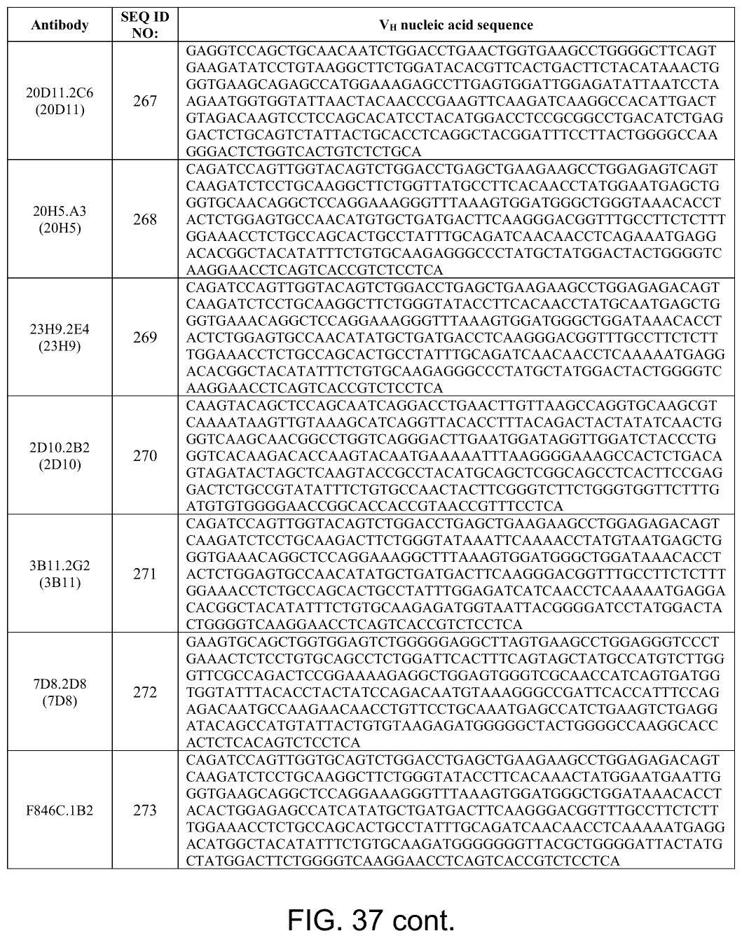

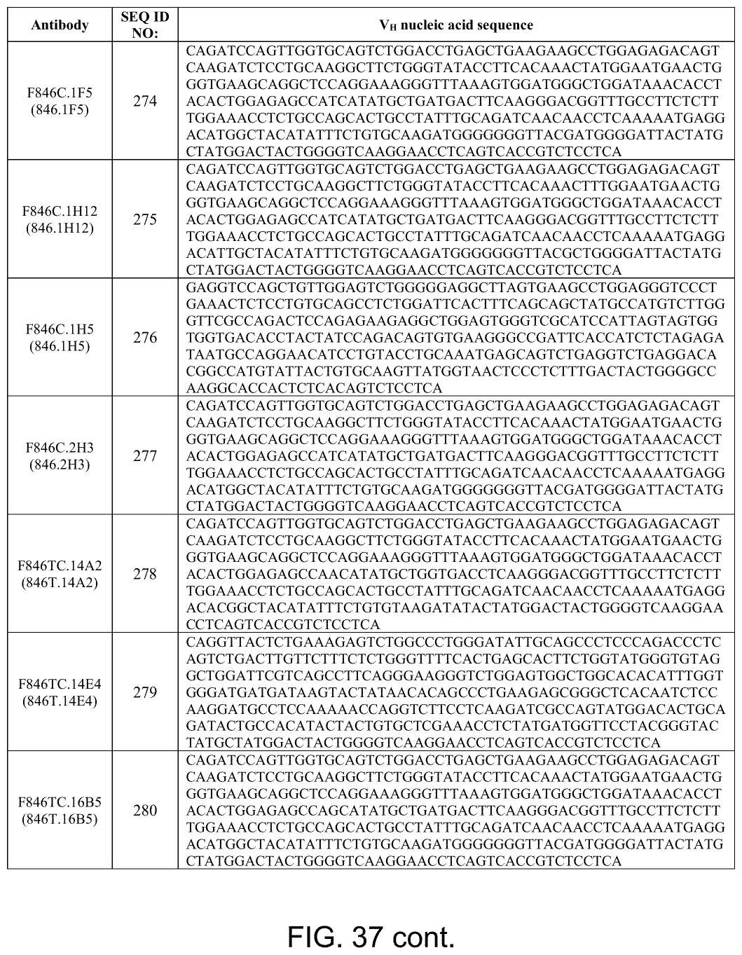

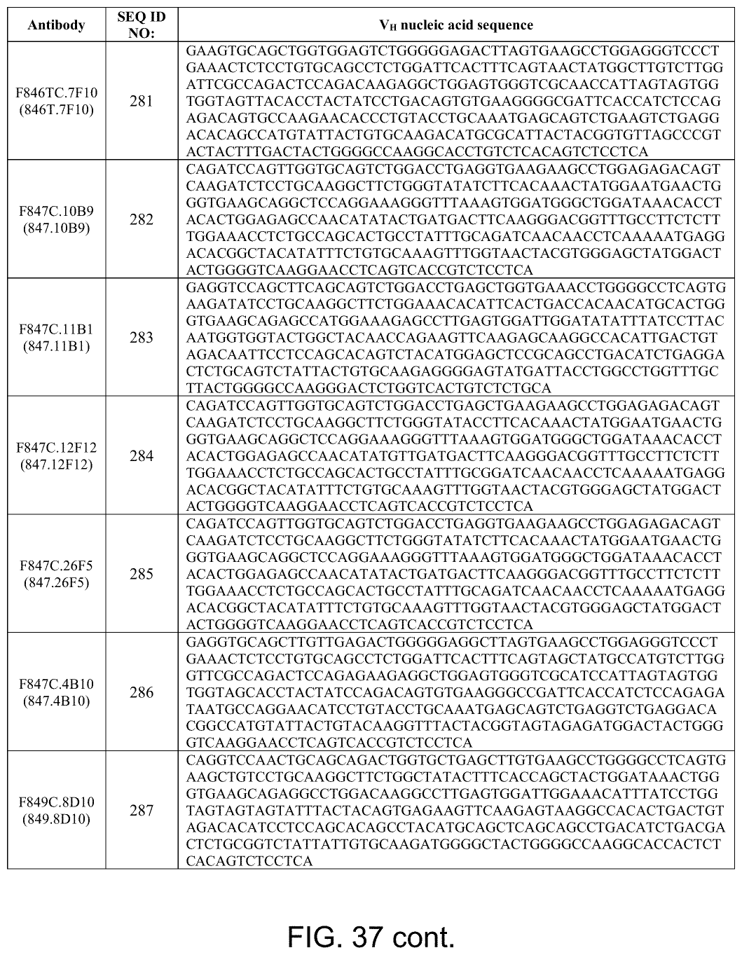

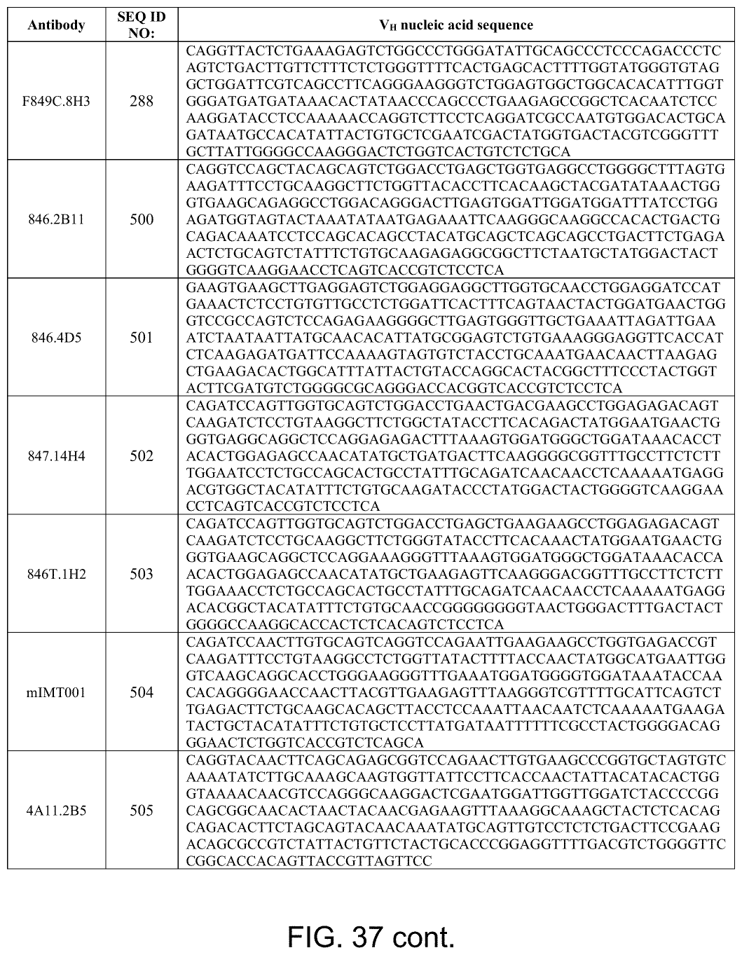

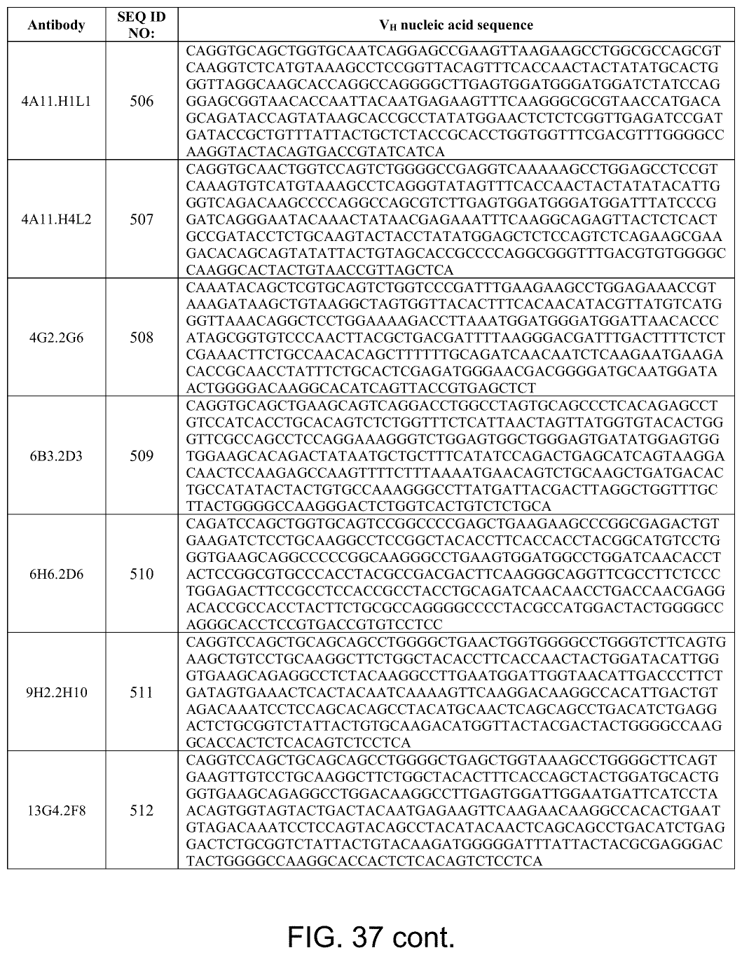

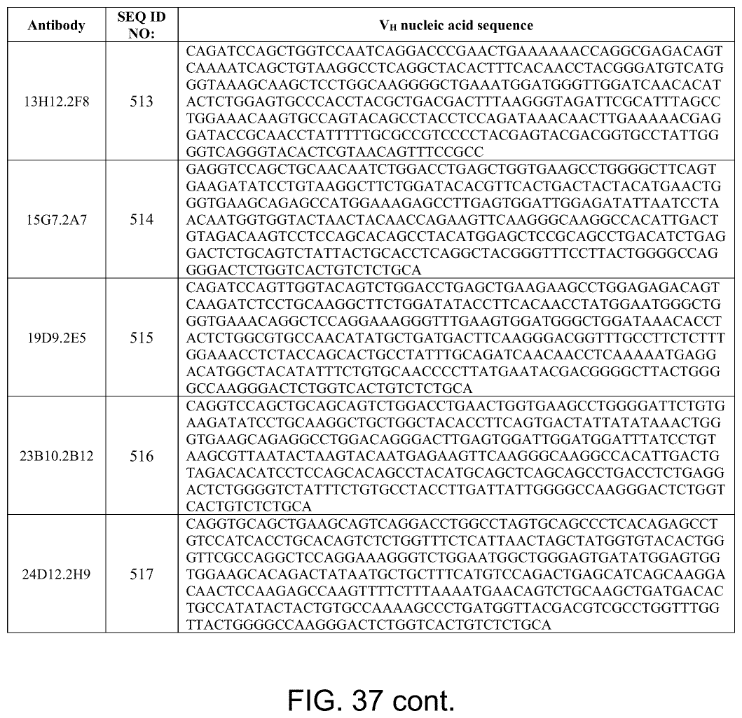

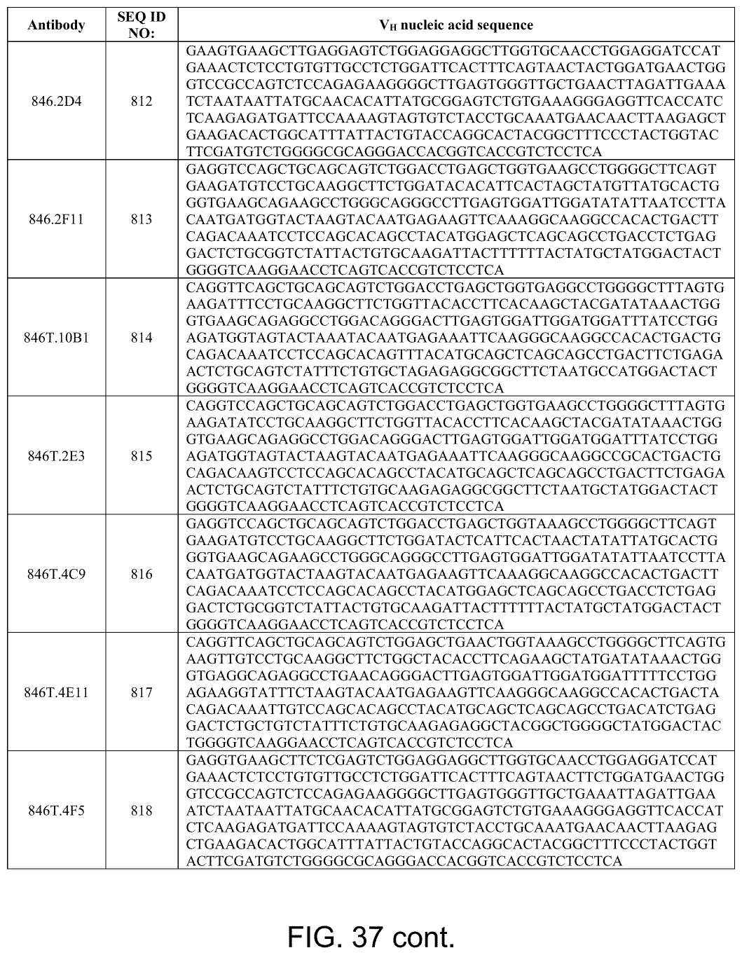

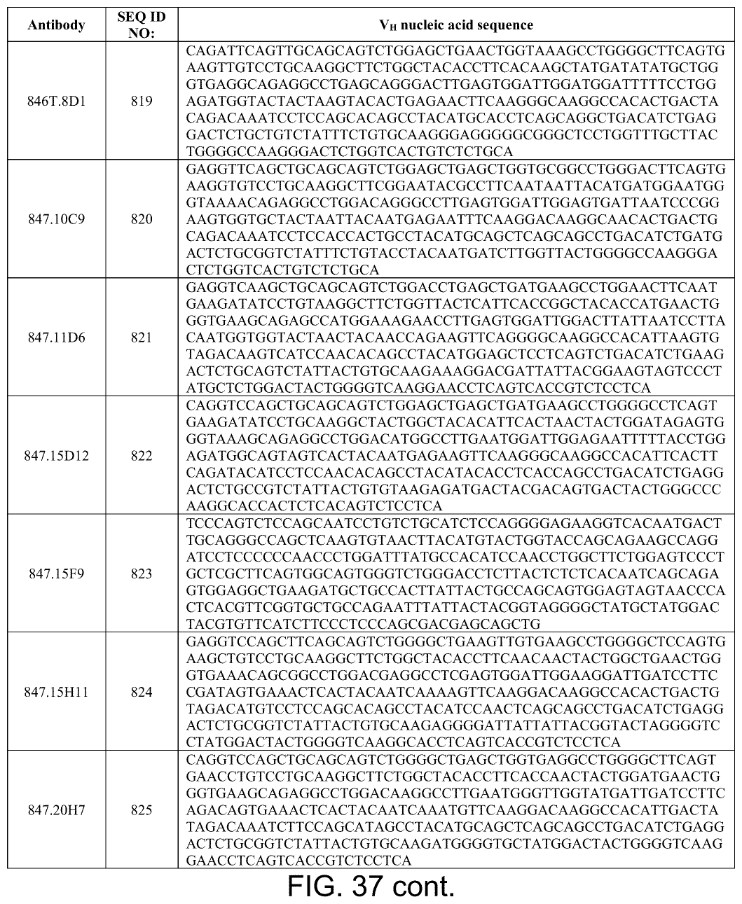

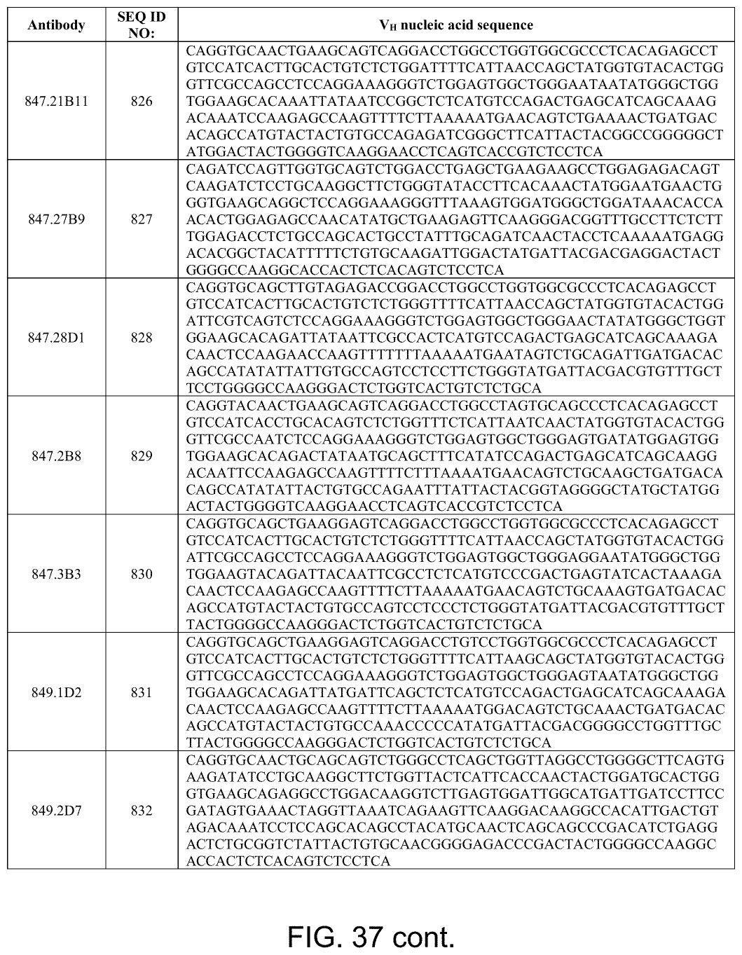

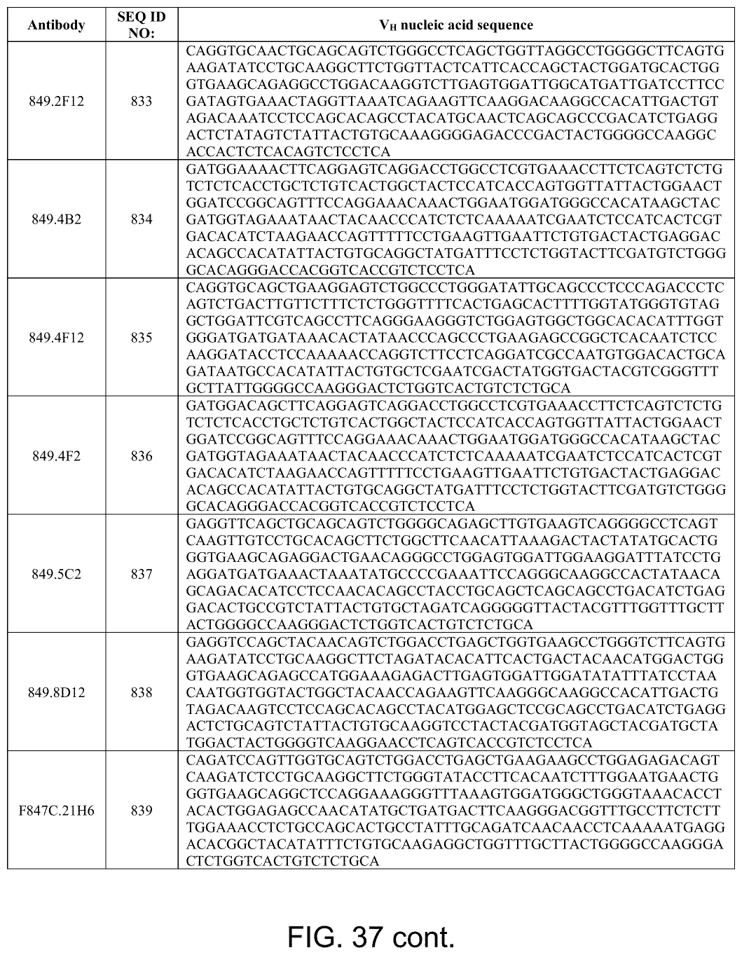

Also disclosed herein are proteins comprising one or more peptide sequences having at least 80%, 85%, 90%, 95%, 99%, or 100% homology to one or more of the peptide sequences of FIG. 18-27. In some embodiments, the protein is an antibody or binding fragment thereof. In some embodiments, the protein comprises a) a V_{H}-CDR1 peptide sequence having at least 80%, 85%, 90%, 95%, 99%, or 100% homology to one or more of the peptide sequences of FIG. 18; b) a V_{H}-CDR2 peptide sequence having at least 80%, 85%, 90%, 95%, 99%, or 100% homology to one or more of the peptide sequences of FIG. 19; c) a V_{H}-CDR3 peptide sequence having at least 80%, 85%, 90%, 95%, 99%, or 100% homology to one or more of the peptide sequences of FIG. 20; d) a V_{L}-CDR1 peptide sequence having at least 80%, 85%, 90%, 95%, 99%, or 100% homology to one or more of the peptide sequences of FIG. 21; e) a V_{L}-CDR2 peptide sequence having at least 80%, 85%, 90%, 95%, 99%, or 100% homology to one or more of the peptide sequences of FIG. 22; f) a V_{L}-CDR3 peptide sequence having at least 80%, 85%, 90%, 95%, 99%, or 100% homology to one or more of the peptide sequences of FIG. 23; g) a heavy chain variable region peptide sequence having at least 80%, 85%, 90%, 95%, 99%, or 100% homology to one or more of the peptide sequences of FIG. 24; h) a light chain variable region peptide sequence having at least 80%, 85%, 90%, 95%, 99%, or 100% homology to one or more of the peptide sequences of FIG. 25; i) a heavy chain peptide sequence having at least 80%, 85%, 90%, 95%, 99%, or 100% homology to one or more of the peptide sequences of FIG. 26; j) a light chain peptide sequence having at least 80%, 85%, 90%, 95%, 99%, or 100% homology to one or more of the peptide sequences of FIG. 27; or any combination thereof, including 1 of the provided sequences or combinations of 1, 2, 3, 4, 5, 6, 7, 8, 9, or 10 of the provided sequences. In some embodiments, the protein comprises a peptide sequence having at least 80%, 85%, 90%, 95%, 99%, or 100% homology to a peptide sequence encoded by any one or more of the nucleic acid sequences of FIG. 37-40. In some embodiments, the protein is an antibody or binding fragment thereof that binds to Gal3.

### Methods of Use

In some embodiments, any of the constructs provided herein can be used for neurological disorders and/or proteopathies.

Disclosed herein are methods of treating a neurological disorder in a subject in need thereof. The methods comprise administering to the subject an effective amount of an anti-Gal3 antibody or binding fragment thereof, thereby treating the neurological disorder. In some embodiments, the methods further comprise selecting the subject as having the neurological disorder or at risk of contracting the neurological disorder prior to the administering step. In some embodiments, the methods further comprise detecting an amelioration of symptoms associated with the neurological disorder after the administering step. In some embodiments, the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or any combination thereof. In some embodiments, the neurological disorder is Alzheimer's disease, and wherein the anti-Gal3 antibody or binding fragment thereof disrupts binding between Gal3 and amyloid precursor protein (APP) or amyloid beta (Aβ), or both. In some embodiments, the APP comprises the sequence of APP695 (SEQ ID NO: 2). In some embodiments, the Aβ comprises Aβ monomers, Aβ oligomers, Aβ fibrils, or any combination thereof. In some embodiments, the Aβ comprises the sequence of Aβ42 (SEQ ID NO: 244). In some embodiments, the anti-Gal3 antibody or binding fragment thereof reduces the binding between Gal3 and APP or Aβ, or both, by at least 50%, 55%, 60%, 65%, 70%, 75%, 80%, 85%, 90%, 95%, 96%, 97%, 98%, or 99%, or any percentage within a range defined by any two aforementioned percentages. In some embodiments, the anti-Gal3 antibody or binding fragment thereof promotes phagocytic function of microglia in the subject. In some embodiments, the anti-Gal3 antibody or binding fragment thereof inhibits AD-mediated activation of microglia in the subject. In some embodiments, the AD-mediated activation of microglia is inhibited by at least 50%, 55%, 60%, 65%, 70%, 75%, 80%, 85%, 90%, 95%, 96%, 97%, 98%, or 99%, or any percentage within a range defined by any two aforementioned percentages. In some embodiments, the anti-Gal3 antibody or binding fragment thereof inhibits Aβ fibril or oligomer formation in the subject. In some embodiments, the Aβ fibril or oligomer formation is inhibited by at least 50%, 55%, 60%, 65%, 70%, 75%, 80%, 85%, 90%, 95%, 96%, 97%, 98%, or 99%, or any percentage within a range defined by any two aforementioned percentages. In some embodiments, the anti-Gal3 antibody or binding fragment thereof promotes neuronal regeneration in the subject. In some embodiments, the anti-Gal3 antibody or binding fragment thereof disrupts binding between Gal3 and Toll-like receptor 4 (TLR4) or triggering receptor expressed on myeloid cells 2 (TREM2), or both. In some embodiments, the binding between Gal3 and TLR4 or TREM2, or both, is disrupted by at least 50%, 55%, 60%, 65%, 70%, 75%, 80%, 85%, 90%, 95%, 96%, 97%, 98%, or 99%, or any percentage within a range defined by any two aforementioned percentages. In some embodiments, more than one anti-Gal3 antibody or binding fragment thereof is administered to the subject. In some embodiments, the anti-Gal3 antibody or binding fragment thereof is administered with one or more additional therapeutic compositions. In some embodiments, the one or more additional therapeutic compositions comprise a cholinesterase inhibitor, an NMDA receptor antagonist, or both. In some embodiments, the cholinesterase inhibitor comprises tacrine, rivastigmine, galantamine, donepezil, or any combination thereof. In some embodiments, the NMDA receptor antagonist comprises memantine.

Also disclosed herein are methods of disrupting binding between Gal3 and APP or Aβ, or both. In some embodiments, the methods comprise contacting the APP or Aβ, or both, with an anti-Gal3 antibody or binding fragment thereof, thereby disrupting the binding between Gal3 and APP. In some embodiments, the APP or Aβ, or both, is soluble or part of a first cell. In some embodiments, the Gal3 is soluble or part of a second cell. In some embodiments, the APP comprises the sequence of APP695 (SEQ ID NO: 2). In some embodiments, the Aβ comprises Aβ monomers, Aβ oligomers, Aβ fibrils, or any combination thereof. In some embodiments, the Aβ comprises the sequence of Aβ42 (SEQ ID NO: 244). In some embodiments, the anti-Gal3 antibody or binding fragment thereof reduces the binding between Gal3 and APP or Aβ, or both, by at least 50%, 55%, 60%, 65%, 70%, 75%, 80%, 85%, 90%, 95%, 96%, 97%, 98%, or 99%, or any percentage within a range defined by any two aforementioned percentages. In some embodiments, the anti-Gal3 antibody or binding fragment thereof reduces the binding between Gal3 and APP or Aβ, or both, by at least 85%. In some embodiments, the anti-Gal3 antibody or binding fragment thereof reduces the binding between Gal3 and APP or Aβ, or both, by at least 90%. In some embodiments, the anti-Gal3 antibody or binding fragment thereof reduces the binding between Gal3 and APP or Aβ, or both, by at least 95%. In some embodiments, the APP is contacted with more than one anti-Gal3 antibody or binding fragment thereof. In some embodiments, the Aβ is Aβ peptide or Aβ aggregates, or both. In some embodiments, the Aβ aggregates are Aβ fibrils or Aβ oligomers, or both.

Also disclosed herein are methods of treating a proteopathy in a subject in need thereof. In some embodiments, the methods comprise administering to the subject an effective amount of an anti-Gal3 antibody or binding fragment thereof, thereby treating the proteopathy in the subject. In some embodiments, the methods further comprise selecting the subject as having the proteopathy or at risk of contracting the proteopathy prior to the administering step. In some embodiments, the methods further comprise detecting an amelioration of symptoms associated with the proteopathy after the administering step. In some embodiments, treating the proteopathy comprises treating an active proteopathy, or a prophylactic treatment, or both, in the subject. In some embodiments, the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof. In some embodiments, more than one anti-Gal3 antibody or binding fragment thereof is administered to the subject. In some embodiments, the anti-Gal3 antibody or binding fragment thereof is administered with one or more additional therapeutic compositions. In some embodiments, the one or more additional therapeutic compositions comprise a cholinesterase inhibitor, an NMDA receptor antagonist, insulin, or any combination thereof. In some embodiments, the cholinesterase inhibitor comprises tacrine, rivastigmine, galantamine, donepezil or any combination thereof. In some embodiments, the NMDA receptor antagonist comprises memantine.

Also disclosed herein are methods of administering an antibody to a subject. In some embodiments, the methods comprise administering to the subject an anti-Gal3 antibody or binding fragment thereof. In some embodiments, the methods further comprise selecting the subject as having a neurological disease or a proteopathy or at risk of contracting the neurological disease or the proteopathy prior to the administering step. In some embodiments, the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof. In some embodiments, the neurological disorder is Alzheimer's disease. In some embodiments, the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof. In some embodiments, more than one anti-Gal3 antibody or binding fragment thereof is administered to the subject.

Also disclosed herein are methods of treating brain cancer in a subject in need thereof. In some embodiments, the methods comprise administering to the subject an effective amount of an anti-Gal3 antibody or binding fragment thereof, thereby treating the brain cancer in the subject. In some embodiments, the anti-Gal3 antibody or binding fragment thereof is capable of cross the blood-brain barrier. In some embodiments, administration of the anti-Gal3 antibody or binding fragment thereof induces apoptosis in the brain cancer.

Also disclosed herein are methods of promoting neuronal regeneration in a subject in need thereof. In some embodiments, the methods comprise administering to the subject an effective amount of an anti-Gal3 antibody or binding fragment thereof, thereby promoting neuronal regeneration in the subject. In some embodiments, the methods further comprise selecting the subject as having neuronal degeneration or at risk of having neuronal degeneration prior to the administering step. In some embodiments, the methods further comprise detecting the neuronal regeneration in the subject after the administering step. In some embodiments, the subject comprises neuronal degeneration associated with inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof. In some embodiments, the neuronal degeneration is associated with Alzheimer's disease, and wherein the anti-Gal3 antibody or binding fragment thereof disrupts binding between Gal3 and amyloid precursor protein (APP) or amyloid beta (Aβ), or both. In some embodiments, more than one anti-Gal3 antibody or binding fragment thereof is administered to the subject.

As applied to any of the methods disclosed herein, in some embodiments, the anti-Gal3 antibody or binding fragment thereof is administered enterally, orally, intranasally, parenterally, intracranially, subcutaneously, intramuscularly, intradermally, or intravenously, or any combination thereof.

As applied to any of the methods disclosed herein, in some embodiments, the anti-Gal3 antibody or binding fragment thereof binds to one or more peptides of SEQ ID NOs: 3-26. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof belongs to bin 3, 8, 17, or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof competes with an antibody that belongs to bins 3, 8, 17 or 24 for binding to Gal3.

As applied to any of the methods or uses disclosed herein, in some embodiments, the anti-Gal3 antibody or binding fragment thereof comprises any one or more sequences (such as a V_{H}-CDR1, V_{H}-CDR2, V_{H}-CDR3, V_{L}-CDR1, V_{L}-CDR2, V_{L}-CDR3, heavy chain variable region, light chain variable region, heavy chain, or light chain sequence) provided throughout this disclosure. In some embodiments, the anti-Gal3 antibody or binding fragment thereof comprises any one or more sequences as shown in FIG. 18-32, including any one or more CDRs, heavy chain variable regions, light chain variable regions, heavy chains, light chains, combinations of CDRs, combinations of variable regions, or combinations of heavy chain and light chain described therein. In some embodiments, the anti-Gal3 antibody or binding fragment thereof comprises a peptide sequence having at least 80%, 85%, 90%, 95%, 99%, or 100% homology to the peptide sequence encoded by any one or more of the nucleic acid sequences as shown in FIG. 37-40, including any nucleic sequences encoding for a heavy chain variable region, light chain variable region, heavy chain, or light chain.

As applied to any of the methods disclosed herein, in some embodiments, the anti-Gal3 antibody or binding fragment thereof comprises (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 27-44, 245-246, 588-615, the V_{H}-CDR2 comprises an amino acid sequence selected from SEQ ID NOs: 45-60, 247-248, 616-643, the V_{H}-CDR3 comprises an amino acid sequence selected from SEQ ID NOs: 61-81, 249-250, 644-671, the V_{L}-CDR1 comprises an amino acid sequence selected from SEQ ID NOs: 82-101, 251-252, 672-699, the V_{L}-CDR2 comprises an amino acid sequence selected from SEQ ID NOs: 102-116, 253, 700-727, and the V_{L}-CDR3 comprises an amino acid sequence selected from SEQ ID NOs: 117-135, 254-255, 728-755. In some embodiments, any of the methods disclosed herein involving an anti-Gal3 antibody or binding fragment can be performed with an antigen binding molecule that binds to Gal3.

As applied to any of the methods disclosed herein, in some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23. In some embodiments, any of the methods disclosed herein involving an anti-Gal3 antibody or binding fragment can be performed with an antigen binding molecule that binds to Gal3.

As applied to any of the methods disclosed herein, in some embodiments, the heavy chain variable region (V_{H}) comprises an amino acid sequence having at least 75%, 80%, 85%, 90%, 95%, or 100% sequence identity to any sequence according to SEQ ID NOs: 136-160, 256-257, 756-783. In some embodiments, the heavy chain variable region is selected from the group consisting of SEQ ID NOs: 136-160, 256-257, 756-783. In some embodiments, exemplary V_{H} are depicted in FIG. 24. In some embodiments, any of the methods disclosed herein involving an anti-Gal3 antibody or binding fragment can be performed with an antigen binding molecule that binds to Gal3.

As applied to any of the methods disclosed herein, in some embodiments, the light chain variable region (V_{L}) comprises an amino acid sequence having at least 75%, 80%, 85%, 90%, 95%, or 100% sequence identity to any sequence according to SEQ ID NOs: 161-187, 258-259, 784-811. In some embodiments, the light chain variable region is selected from the group consisting of SEQ ID NOs: 161-187, 258-259, 784-811. In some embodiments, exemplary VL are depicted in FIG. 25. In some embodiments, any of the methods disclosed herein involving an anti-Gal3 antibody or binding fragment can be performed with an antigen binding molecule that binds to Gal3.

As applied to any of the methods disclosed herein, in some embodiments, the anti-Gal3 antibody or binding fragment thereof comprises: 1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161; 2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162; 3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163; 4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164; 5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165; 6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166; 7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167; 8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168; 9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169; 10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170; 11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171; 12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172; 13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173; 14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175; 16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176; 17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177; 18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178; 19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179; 20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180; 21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181; 22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182; 23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183; 24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184; 25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185; 26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186; 27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187; 28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258; 29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259; 30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811. In some embodiments, exemplary combinations of heavy chain variable region CDRs are depicted in FIG. 28. In some embodiments, exemplary combinations of light chain variable region CDRs are depicted in FIG. 29. In some embodiments, any of the methods disclosed herein involving an anti-Gal3 antibody or binding fragment can be performed with an antigen binding molecule that binds to Gal3.

As applied to any of the methods disclosed herein, in some embodiments, the anti-Gal3 antibody or binding fragment thereof comprises: 1) the heavy chain variable region of SEQ ID NO: 136 and the light chain variable region of SEQ ID NO: 161; 2) the heavy chain variable region of SEQ ID NO: 137 and the light chain variable region of SEQ ID NO: 162; 3) the heavy chain variable region of SEQ ID NO: 138 and the light chain variable region of SEQ ID NO: 163; 4) the heavy chain variable region of SEQ ID NO: 139 and the light chain variable region of SEQ ID NO: 164; 5) the heavy chain variable region of SEQ ID NO: 140 and the light chain variable region of SEQ ID NO: 165; 6) the heavy chain variable region of SEQ ID NO: 141 and the light chain variable region of SEQ ID NO: 166; 7) the heavy chain variable region of SEQ ID NO: 142 and the light chain variable region of SEQ ID NO: 167; 8) the heavy chain variable region of SEQ ID NO: 143 and the light chain variable region of SEQ ID NO: 168; 9) the heavy chain variable region of SEQ ID NO: 144 and the light chain variable region of SEQ ID NO: 169; 10) the heavy chain variable region of SEQ ID NO: 145 and the light chain variable region of SEQ ID NO: 170; 11) the heavy chain variable region of SEQ ID NO: 139 and the light chain variable region of SEQ ID NO: 171; 12) the heavy chain variable region of SEQ ID NO: 146 and the light chain variable region of SEQ ID NO: 172; 13) the heavy chain variable region of SEQ ID NO: 147 and the light chain variable region of SEQ ID NO: 173; 14) the heavy chain variable region of SEQ ID NO: 148 and the light chain variable region of SEQ ID NO: 174; 15) the heavy chain variable region of SEQ ID NO: 149 and the light chain variable region of SEQ ID NO: 175; 16) the heavy chain variable region of SEQ ID NO: 150 and the light chain variable region of SEQ ID NO: 176; 17) the heavy chain variable region of SEQ ID NO: 151 and the light chain variable region of SEQ ID NO: 177; 18) the heavy chain variable region of SEQ ID NO: 152 and the light chain variable region of SEQ ID NO: 178; 19) the heavy chain variable region of SEQ ID NO: 153 and the light chain variable region of SEQ ID NO: 179; 20) the heavy chain variable region of SEQ ID NO: 154 and the light chain variable region of SEQ ID NO: 180; 21) the heavy chain variable region of SEQ ID NO: 155 and the light chain variable region of SEQ ID NO: 181; 22) the heavy chain variable region of SEQ ID NO: 156 and the light chain variable region of SEQ ID NO: 182; 23) the heavy chain variable region of SEQ ID NO: 157 and the light chain variable region of SEQ ID NO: 183; 24) the heavy chain variable region of SEQ ID NO: 155 and the light chain variable region of SEQ ID NO: 184; 25) the heavy chain variable region of SEQ ID NO: 158 and the light chain variable region of SEQ ID NO: 185; 26) the heavy chain variable region of SEQ ID NO: 159 and the light chain variable region of SEQ ID NO: 186; 27) the heavy chain variable region of SEQ ID NO: 160 and the light chain variable region of SEQ ID NO: 187; 28) the heavy chain variable region of SEQ ID NO: 256 and the light chain variable region of SEQ ID NO: 258; 29) the heavy chain variable region of SEQ ID NO: 257 and the light chain variable region of SEQ ID NO: 259; 30) the heavy chain variable region of SEQ ID NO: 756 and the light chain variable region of SEQ ID NO: 784; 31) the heavy chain variable region of SEQ ID NO: 757 and the light chain variable region of SEQ ID NO: 785; 32) the heavy chain variable region of SEQ ID NO: 758 and the light chain variable region of SEQ ID NO: 786; 33) the heavy chain variable region of SEQ ID NO: 759 and the light chain variable region of SEQ ID NO: 787; 34) the heavy chain variable region of SEQ ID NO: 760 and the light chain variable region of SEQ ID NO: 788; 35) the heavy chain variable region of SEQ ID NO: 761 and the light chain variable region of SEQ ID NO: 789; 36) the heavy chain variable region of SEQ ID NO: 762 and the light chain variable region of SEQ ID NO: 790; 37) the heavy chain variable region of SEQ ID NO: 763 and the light chain variable region of SEQ ID NO: 791; 38) the heavy chain variable region of SEQ ID NO: 764 and the light chain variable region of SEQ ID NO: 792; 39) the heavy chain variable region of SEQ ID NO: 765 and the light chain variable region of SEQ ID NO: 793; 40) the heavy chain variable region of SEQ ID NO: 766 and the light chain variable region of SEQ ID NO: 794; 41) the heavy chain variable region of SEQ ID NO: 767 and the light chain variable region of SEQ ID NO: 795; 42) the heavy chain variable region of SEQ ID NO: 768 and the light chain variable region of SEQ ID NO: 796; 43) the heavy chain variable region of SEQ ID NO: 769 and the light chain variable region of SEQ ID NO: 797; 44) the heavy chain variable region of SEQ ID NO: 770 and the light chain variable region of SEQ ID NO: 798; 45) the heavy chain variable region of SEQ ID NO: 771 and the light chain variable region of SEQ ID NO: 799; 46) the heavy chain variable region of SEQ ID NO: 772 and the light chain variable region of SEQ ID NO: 800; 47) the heavy chain variable region of SEQ ID NO: 773 and the light chain variable region of SEQ ID NO: 801; 48) the heavy chain variable region of SEQ ID NO: 774 and the light chain variable region of SEQ ID NO: 802; 49) the heavy chain variable region of SEQ ID NO: 775 and the light chain variable region of SEQ ID NO: 803; 50) the heavy chain variable region of SEQ ID NO: 776 and the light chain variable region of SEQ ID NO: 804; 51) the heavy chain variable region of SEQ ID NO: 777 and the light chain variable region of SEQ ID NO: 805; 52) the heavy chain variable region of SEQ ID NO: 778 and the light chain variable region of SEQ ID NO: 806; 53) the heavy chain variable region of SEQ ID NO: 779 and the light chain variable region of SEQ ID NO: 807; 54) the heavy chain variable region of SEQ ID NO: 780 and the light chain variable region of SEQ ID NO: 808; 55) the heavy chain variable region of SEQ ID NO: 781 and the light chain variable region of SEQ ID NO: 809; 56) the heavy chain variable region of SEQ ID NO: 782 and the light chain variable region of SEQ ID NO: 810; or 57) the heavy chain variable region of SEQ ID NO: 783 and the light chain variable region of SEQ ID NO: 811. In some embodiments, any of the methods disclosed herein involving an anti-Gal3 antibody or binding fragment can be performed with an antigen binding molecule that binds to Gal3.

As applied to any of the methods disclosed herein, in some embodiments, the anti-Gal3 antibody or binding fragment thereof comprises the heavy chain (HC) sequence of any one of SEQ ID NOs: 188-216. In some embodiments, exemplary HC sequences are depicted in FIG. 26. In some embodiments, any of the methods disclosed herein involving an anti-Gal3 antibody or binding fragment can be performed with an antigen binding molecule that binds to Gal3.

As applied to any of the methods disclosed herein, in some embodiments, the anti-Gal3 antibody or binding fragment thereof comprises the light chain (LC) sequence of any one of SEQ ID NOs: 217-243. In some embodiments, exemplary LC sequences are depicted in FIG. 27. In some embodiments, any of the methods disclosed herein involving an anti-Gal3 antibody or binding fragment can be performed with an antigen binding molecule that binds to Gal3.

As applied to any of the methods disclosed herein, in some embodiments, the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. As applied to any of the methods disclosed herein, in some embodiments, the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. In some embodiments, the anti-Gal3 antibody or binding fragment thereof is selected from the group consisting of at least one of: TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof. In some embodiments, the anti-Gal3 antibody or binding fragment thereof disrupts an interaction between Gal3 and an antibody selected from 846.4D5, 15F10.2D6, F846C.1B2, and F846C.1H12. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32. In some embodiments, any of the methods disclosed herein involving an anti-Gal3 antibody or binding fragment can be performed with an antigen binding molecule that binds to Gal3.

Also disclosed herein are anti-Gal3 antibodies or binding fragments thereof for use in the treatment of a neurodegenerative disorder in a subject in need thereof. In some embodiments, the neurodegenerative disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof. In some embodiments, the neurological disorder is Alzheimer's disease, and wherein the anti-Gal3 antibody or binding fragment thereof disrupts binding between Gal3 and amyloid precursor protein (APP) or Aβ, or both. In some embodiments, the APP comprises the sequence of APP695 (SEQ ID NO: 2). In some embodiments, the Aβ comprises Aβ monomers, Aβ oligomers, Aβ fibrils, or any combination thereof. In some embodiments, the Aβ comprises the sequence of Aβ42 (SEQ ID NO: 244). In some embodiments, the anti-Gal3 antibody or binding fragment thereof reduces the binding between Gal3 and APP or Aβ, or both, by at least 50%, 55%, 60%, 65%, 70%, 75%, 80%, 85%, 90%, 95%, 96%, 97%, 98%, or 99%, or any percentage within a range defined by any two aforementioned percentages. In some embodiments, the anti-Gal3 antibody or binding fragment thereof promotes phagocytic function of microglia in the subject. In some embodiments, the anti-Gal3 antibody or binding fragment thereof inhibits AD-mediated activation of microglia in the subject. In some embodiments, the AD-mediated activation of microglia is inhibited by at least 50%, 55%, 60%, 65%, 70%, 75%, 80%, 85%, 90%, 95%, 96%, 97%, 98%, or 99%, or any percentage within a range defined by any two aforementioned percentages. In some embodiments, the anti-Gal3 antibody or binding fragment thereof inhibits Aβ fibril or oligomer formation in the subject. In some embodiments, the Aβ fibril or oligomer formation is inhibited by at least 50%, 55%, 60%, 65%, 70%, 75%, 80%, 85%, 90%, 95%, 96%, 97%, 98%, or 99%, or any percentage within a range defined by any two aforementioned percentages. In some embodiments, the anti-Gal3 antibody or binding fragment thereof disrupts an interaction between Gal3 and Toll-like receptor 4 (TLR4) or triggering receptor expressed on myeloid cells 2 (TREM2), or both. In some embodiments, the interaction between Gal3 and TLR4 or TREM2, or both is disrupted by at least 50%, 55%, 60%, 65%, 70%, 75%, 80%, 85%, 90%, 95%, 96%, 97%, 98%, or 99%, or any percentage within a range defined by any two aforementioned percentages.

Also disclosed herein are anti-Gal3 antibodies or binding fragments thereof for use in the treatment of a proteopathy in a subject in need thereof. In some embodiments, the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof.

Also disclosed herein are anti-Gal3 antibodies or binding fragments thereof for use in promoting neuronal regeneration in a subject in need thereof. In some embodiments, the subject comprises neuronal degeneration associated with inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof. In some embodiments, the neuronal degeneration is associated with Alzheimer's disease, and wherein the anti-Gal3 antibody or binding fragment thereof disrupts binding between Gal3 and amyloid precursor protein (APP) or amyloid beta (Aβ), or both, in the subject. In some embodiments, more than one anti-Gal3 antibody or binding fragment thereof is administered to the subject.

As applied to any of the uses disclosed herein, in some embodiments, the anti-Gal3 antibody or binding fragment thereof binds to one or more peptides of SEQ ID NOs: 3-26. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof belongs to bin 3, 8, 17, or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof competes with an antibody that belongs to bins 3, 8, 17 or 24 for binding to Gal3.

As applied to any of the uses disclosed herein, in some embodiments, the anti-Gal3 antibody or binding fragment thereof comprising (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 27-44, 245-246, 588-615, the V_{H}-CDR2 comprises an amino acid sequence selected from SEQ ID NOs: 45-60, 247-248, 616-643, the V_{H}-CDR3 comprises an amino acid sequence selected from SEQ ID NOs: 61-81, 249-250, 644-671, the V_{L}-CDR1 comprises an amino acid sequence selected from SEQ ID NOs: 82-101, 251-252, 672-699, the V_{L}-CDR2 comprises an amino acid sequence selected from SEQ ID NOs: 102-116, 253, 700-727, and the V_{L}-CDR3 comprises an amino acid sequence selected from SEQ ID NOs: 117-135, 254-255, 728-755. In some embodiments, any of the uses disclosed herein involving an anti-Gal3 antibody or binding fragment can be performed with an antigen binding molecule that binds to Gal3.

As applied to any of the uses disclosed herein, in some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23. In some embodiments, any of the uses disclosed herein involving an anti-Gal3 antibody or binding fragment can be performed with an antigen binding molecule that binds to Gal3.

As applied to any of the uses disclosed herein, in some embodiments, the heavy chain variable region (V_{H}) comprises an amino acid sequence having at least 75%, 80%, 85%, 90%, 95%, or 100% sequence identity to any sequence according to SEQ ID NOs: 136-160, 256-257, 756-783. In some embodiments, the heavy chain variable region is selected from the group consisting of SEQ ID NOs: 136-160, 256-257, 756-783. In some embodiments, exemplary V_{H} are depicted in FIG. 24. In some embodiments, any of the uses disclosed herein involving an anti-Gal3 antibody or binding fragment can be performed with an antigen binding molecule that binds to Gal3.

As applied to any of the uses disclosed herein, in some embodiments, the light chain variable region (V_{L}) comprises an amino acid sequence having at least 75%, 80%, 85%, 90%, 95%, or 100% sequence identity to any sequence according to SEQ ID NOs: 161-187, 258-259, 784-811. In some embodiments, the light chain variable region is selected from the group consisting of SEQ ID NOs: 161-187, 258-259, 784-811. In some embodiments, exemplary V_{L} are depicted in FIG. 25. In some embodiments, any of the uses disclosed herein involving an anti-Gal3 antibody or binding fragment can be performed with an antigen binding molecule that binds to Gal3.

As applied to any of the uses disclosed herein, in some embodiments, the anti-Gal3 antibody or binding fragment thereof comprises: 1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161; 2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162; 3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163; 4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164; 5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165; 6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166; 7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167; 8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168; 9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169; 10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170; 11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171; 12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172; 13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173; 14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175; 16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176; 17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177; 18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178; 19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179; 20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180; 21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181; 22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182; 23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183; 24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184; 25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185; 26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186; 27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187; 28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258; 29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259; 30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811. In some embodiments, exemplary combinations of heavy chain variable region CDRs are depicted in FIG. 28. In some embodiments, exemplary combinations of light chain variable region CDRs are depicted in FIG. 29. In some embodiments, any of the uses disclosed herein involving an anti-Gal3 antibody or binding fragment can be performed with an antigen binding molecule that binds to Gal3.

As applied to any of the uses disclosed herein, in some embodiments, the anti-Gal3 antibody or binding fragment thereof comprises: 1) the heavy chain variable region of SEQ ID NO: 136 and the light chain variable region of SEQ ID NO: 161; 2) the heavy chain variable region of SEQ ID NO: 137 and the light chain variable region of SEQ ID NO: 162; 3) the heavy chain variable region of SEQ ID NO: 138 and the light chain variable region of SEQ ID NO: 163; 4) the heavy chain variable region of SEQ ID NO: 139 and the light chain variable region of SEQ ID NO: 164; 5) the heavy chain variable region of SEQ ID NO: 140 and the light chain variable region of SEQ ID NO: 165; 6) the heavy chain variable region of SEQ ID NO: 141 and the light chain variable region of SEQ ID NO: 166; 7) the heavy chain variable region of SEQ ID NO: 142 and the light chain variable region of SEQ ID NO: 167; 8) the heavy chain variable region of SEQ ID NO: 143 and the light chain variable region of SEQ ID NO: 168; 9) the heavy chain variable region of SEQ ID NO: 144 and the light chain variable region of SEQ ID NO: 169; 10) the heavy chain variable region of SEQ ID NO: 145 and the light chain variable region of SEQ ID NO: 170; 11) the heavy chain variable region of SEQ ID NO: 139 and the light chain variable region of SEQ ID NO: 171; 12) the heavy chain variable region of SEQ ID NO: 146 and the light chain variable region of SEQ ID NO: 172; 13) the heavy chain variable region of SEQ ID NO: 147 and the light chain variable region of SEQ ID NO: 173; 14) the heavy chain variable region of SEQ ID NO: 148 and the light chain variable region of SEQ ID NO: 174; 15) the heavy chain variable region of SEQ ID NO: 149 and the light chain variable region of SEQ ID NO: 175; 16) the heavy chain variable region of SEQ ID NO: 150 and the light chain variable region of SEQ ID NO: 176; 17) the heavy chain variable region of SEQ ID NO: 151 and the light chain variable region of SEQ ID NO: 177; 18) the heavy chain variable region of SEQ ID NO: 152 and the light chain variable region of SEQ ID NO: 178; 19) the heavy chain variable region of SEQ ID NO: 153 and the light chain variable region of SEQ ID NO: 179; 20) the heavy chain variable region of SEQ ID NO: 154 and the light chain variable region of SEQ ID NO: 180; 21) the heavy chain variable region of SEQ ID NO: 155 and the light chain variable region of SEQ ID NO: 181; 22) the heavy chain variable region of SEQ ID NO: 156 and the light chain variable region of SEQ ID NO: 182; 23) the heavy chain variable region of SEQ ID NO: 157 and the light chain variable region of SEQ ID NO: 183; 24) the heavy chain variable region of SEQ ID NO: 155 and the light chain variable region of SEQ ID NO: 184; 25) the heavy chain variable region of SEQ ID NO: 158 and the light chain variable region of SEQ ID NO: 185; 26) the heavy chain variable region of SEQ ID NO: 159 and the light chain variable region of SEQ ID NO: 186; 27) the heavy chain variable region of SEQ ID NO: 160 and the light chain variable region of SEQ ID NO: 187; 28) the heavy chain variable region of SEQ ID NO: 256 and the light chain variable region of SEQ ID NO: 258; 29) the heavy chain variable region of SEQ ID NO: 257 and the light chain variable region of SEQ ID NO: 259; 30) the heavy chain variable region of SEQ ID NO: 756 and the light chain variable region of SEQ ID NO: 784; 31) the heavy chain variable region of SEQ ID NO: 757 and the light chain variable region of SEQ ID NO: 785; 32) the heavy chain variable region of SEQ ID NO: 758 and the light chain variable region of SEQ ID NO: 786; 33) the heavy chain variable region of SEQ ID NO: 759 and the light chain variable region of SEQ ID NO: 787; 34) the heavy chain variable region of SEQ ID NO: 760 and the light chain variable region of SEQ ID NO: 788; 35) the heavy chain variable region of SEQ ID NO: 761 and the light chain variable region of SEQ ID NO: 789; 36) the heavy chain variable region of SEQ ID NO: 762 and the light chain variable region of SEQ ID NO: 790; 37) the heavy chain variable region of SEQ ID NO: 763 and the light chain variable region of SEQ ID NO: 791; 38) the heavy chain variable region of SEQ ID NO: 764 and the light chain variable region of SEQ ID NO: 792; 39) the heavy chain variable region of SEQ ID NO: 765 and the light chain variable region of SEQ ID NO: 793; 40) the heavy chain variable region of SEQ ID NO: 766 and the light chain variable region of SEQ ID NO: 794; 41) the heavy chain variable region of SEQ ID NO: 767 and the light chain variable region of SEQ ID NO: 795; 42) the heavy chain variable region of SEQ ID NO: 768 and the light chain variable region of SEQ ID NO: 796; 43) the heavy chain variable region of SEQ ID NO: 769 and the light chain variable region of SEQ ID NO: 797; 44) the heavy chain variable region of SEQ ID NO: 770 and the light chain variable region of SEQ ID NO: 798; 45) the heavy chain variable region of SEQ ID NO: 771 and the light chain variable region of SEQ ID NO: 799; 46) the heavy chain variable region of SEQ ID NO: 772 and the light chain variable region of SEQ ID NO: 800; 47) the heavy chain variable region of SEQ ID NO: 773 and the light chain variable region of SEQ ID NO: 801; 48) the heavy chain variable region of SEQ ID NO: 774 and the light chain variable region of SEQ ID NO: 802; 49) the heavy chain variable region of SEQ ID NO: 775 and the light chain variable region of SEQ ID NO: 803; 50) the heavy chain variable region of SEQ ID NO: 776 and the light chain variable region of SEQ ID NO: 804; 51) the heavy chain variable region of SEQ ID NO: 777 and the light chain variable region of SEQ ID NO: 805; 52) the heavy chain variable region of SEQ ID NO: 778 and the light chain variable region of SEQ ID NO: 806; 53) the heavy chain variable region of SEQ ID NO: 779 and the light chain variable region of SEQ ID NO: 807; 54) the heavy chain variable region of SEQ ID NO: 780 and the light chain variable region of SEQ ID NO: 808; 55) the heavy chain variable region of SEQ ID NO: 781 and the light chain variable region of SEQ ID NO: 809; 56) the heavy chain variable region of SEQ ID NO: 782 and the light chain variable region of SEQ ID NO: 810; or 57) the heavy chain variable region of SEQ ID NO: 783 and the light chain variable region of SEQ ID NO: 811. In some embodiments, any of the uses disclosed herein involving an anti-Gal3 antibody or binding fragment can be performed with an antigen binding molecule that binds to Gal3. In some embodiments, the antibody has a sequence that is a consensus sequence of 1, 2, 3, 4, 5, or 6 CDRs of any two or more (e.g., 3, 4, 5, 10, 15, 20, 25, 30, 35, 40, 50, 60, 70 or all) of the antibodies provided herein. In some embodiments, the antibody has a sequence that is a consensus sequence of the VH, VL, or V_{H} and VL of any two or more (e.g., 3, 4, 5, 10, 15, 20, 25, 30, 35, 40, 50, 60, 70 or all) of the antibodies provided herein.

As applied to any of the uses disclosed herein, in some embodiments, the anti-Gal3 antibody or binding fragment thereof comprises the heavy chain (HC) sequence of any one of SEQ ID NOs: 188-216. In some embodiments, exemplary HC sequences are depicted in FIG. 26. In some embodiments, any of the uses disclosed herein involving an anti-Gal3 antibody or binding fragment can be performed with an antigen binding molecule that binds to Gal3.

As applied to any of the uses disclosed herein, in some embodiments, the anti-Gal3 antibody or binding fragment thereof comprises the light chain (LC) sequence of any one of SEQ ID NOs: 217-243. In some embodiments, exemplary LC sequences are depicted in FIG. 27. In some embodiments, any of the uses disclosed herein involving an anti-Gal3 antibody or binding fragment can be performed with an antigen binding molecule that binds to Gal3.

As applied to any of the uses disclosed herein, in some embodiments, the anti-Gal3 antibody or binding fragment thereof is selected from the group consisting of at least one of: TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. As applied to any of the uses disclosed herein, in some embodiments, the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. In some embodiments, the anti-Gal3 antibody or binding fragment thereof is selected from the group consisting of at least one of TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof. In some embodiments, the anti-Gal3 antibody or binding fragment thereof disrupts an interaction between Gal3 and an antibody selected from 846.4D5, 15F10.2D6, F846C.1B2, and F846C.1H12. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32. In some embodiments, any of the uses disclosed herein involving an anti-Gal3 antibody or binding fragment can be performed with an antigen binding molecule that binds to Gal3.

As applied to any of the uses disclosed herein, in some embodiments, the anti-Gal3 antibody or binding fragment thereof is administered enterally, orally, intranasally, parenterally, intracranially, subcutaneously, intramuscularly, intradermally, or intravenously, or any combination thereof. In some embodiments, the subject is a mammal. In some instances, the subject is a human.

### Methods of Use—Blood-Brain Barrier Permeability

In some embodiments, any one of the anti-Gal3 antibodies or binding fragments thereof disclosed herein are able to pass the blood-brain barrier and/or the blood-spinal cord barrier. In some embodiments, this phenomenon can be used by conjugating any one of the anti-Gal3 antibodies or binding fragments thereof that can pass the blood-brain barrier and/or the blood-spinal cord barrier to a payload, to prepare an antibody conjugate. The blood-brain barrier and/or the blood-spinal cord barrier in these embodiments may be the blood-brain barrier and/or the blood-spinal cord barrier of a mammal, such as a mouse, rat, other rodent, cat, dog, rabbit, cow, horse, sheep, pig, goat, or human. In some embodiments, the payload might not normally cross the blood-brain barrier and/or the blood-spinal cord barrier, or not cross it as effectively. The payloads may be used, for example, to have a cytotoxic effect against cancerous cells, treat a disease, or used for diagnosis or detection. In addition to the payloads disclosed herein, any other payload known conventionally in the art may be conjugated to any one of the anti-Gal3 antibodies or binding fragments thereof disclosed herein.

Disclosed herein are antibody conjugates comprising any one of the anti-Gal3 antibodies or binding fragments thereof and a payload conjugated to the anti-Gal3 antibody or binding fragment thereof, wherein the antibody conjugate is able to cross a blood-brain barrier. In some embodiments, the payload is not independently capable of crossing the blood-brain barrier or has low permeability across the blood-brain barrier without being conjugated to the anti-Gal3 antibody or binding fragment thereof. In some embodiments, the barrier is in a subject who has a blood brain barrier that is weakened or altered due to a disease that impacts the blood brain barrier, e.g., that decreases the structural integrity of the barrier.

In some embodiments, the conjugation of the payload to the anti-Gal3 antibody or binding fragment thereof increases the permeability of the payload across the blood-brain barrier by at least 5%, 10%, 20%, 30%, 40%, 50%, 60%, 70%, 80%, 90%, 100%, 200%, 300%, 400%, or 500%, or any increase within a range defined by any two of the aforementioned percentages, compared to the unconjugated payload. In some embodiments, the permeability of the payload across the blood-brain barrier is less than 95%, 90%, 80%, 70%, 60%, 50%, 40%, 30%, 20%, 10%, 9%, 8%, 7%, 6%, 5%, 4%, 3%, 2%, or 1% of the permeability of the antibody conjugate across the blood-brain barrier. In some embodiments, the payload or the anti-Gal3 antibody or binding fragment thereof, or both, is used to treat a neurological disorder. In some embodiments, the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer (primary or secondary brain tumors), or any combination thereof. In some embodiments, the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, protein, enzyme, or any combination thereof. In some embodiments, the payload is the second antibody. In some embodiments, the second antibody is not independently capable of crossing the blood-brain barrier or has low permeability across the blood-brain barrier without being conjugated to the anti-Gal3 antibody or binding fragment thereof. In some embodiments, the blood-brain barrier is a mammalian blood-brain barrier. In some embodiments, the blood-brain barrier is a human blood-brain barrier. In some embodiments, the antibody conjugate is formulated to be administered enterally, orally, intranasally, parenterally, intracranially, subcutaneously, intramuscularly, intradermally, or intravenously, or any combination thereof.

As applied to any of the antibody conjugates, in some embodiments, the anti-Gal3 antibody or binding fragment thereof binds to one or more peptides of SEQ ID NOs: 3-26. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof belongs to bin 3, 8, 17, or 24. In some embodiments, the anti-Gal3 antibody or binding fragment thereof disrupts an interaction between Gal3 and an antibody that belongs to bin 3, 8, 17, or 24. In some embodiments, the interaction is disrupted by at least 50%, 55%, 60%, 65%, 70%, 75%, 80%, 85%, 90%, 95%, or 99%, or any percentage within a range defined by any two of the aforementioned percentages. In some embodiments, the anti-Gal3 antibody or binding fragment thereof competes with an antibody that belongs to bins 3, 8, 17 or 24 for binding to Gal3.

As applied to any of the antibody conjugates, in some embodiments, the anti-Gal3 antibody or binding fragment thereof comprises any one or more sequences (such as a V_{H}-CDR1, V_{H}-CDR2, V_{H}-CDR3, V_{L}-CDR1, V_{L}-CDR2, V_{L}-CDR3, heavy chain variable region, light chain variable region, heavy chain, or light chain sequence) provided throughout this disclosure. In some embodiments, the anti-Gal3 antibody or binding fragment thereof comprises any one or more sequences as shown in FIG. 18-32, including any one or more CDRs, heavy chain variable regions, light chain variable regions, heavy chains, light chains, combinations of CDRs, combinations of variable regions, or combinations of heavy chain and light chain described therein. In some embodiments, the anti-Gal3 antibody or binding fragment thereof comprises a peptide sequence having at least 80%, 85%, 90%, 95%, 99%, or 100% homology to the peptide sequence encoded by any one or more of the nucleic acid sequences as shown in FIG. 37-40, including any nucleic sequences encoding for a heavy chain variable region, light chain variable region, heavy chain, or light chain.

As applied to any of the antibody conjugates, in some embodiments, the anti-Gal3 antibody or binding fragment thereof comprises (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 27-44, 245-246, 588-615, the V_{H}-CDR2 comprises an amino acid sequence selected from SEQ ID NOs: 45-60, 247-248, 616-643, the V_{H}-CDR3 comprises an amino acid sequence selected from SEQ ID NOs: 61-81, 249-250, 644-671, the V_{L}-CDR1 comprises an amino acid sequence selected from SEQ ID NOs: 82-101, 251-252, 672-699, the V_{L}-CDR2 comprises an amino acid sequence selected from SEQ ID NOs: 102-116, 253, 700-727, and the V_{L}-CDR3 comprises an amino acid sequence selected from SEQ ID NOs: 117-135, 254-255, 728-755.

As applied to any of the antibody conjugates, in some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23.

As applied to any of the antibody conjugates, in some embodiments, the heavy chain variable region (V_{H}) comprises an amino acid sequence having at least 75%, 80%, 85%, 90%, 95%, or 100% sequence identity to any sequence according to SEQ ID NOs: 136-160, 256-257. In some embodiments, the heavy chain variable region is selected from the group consisting of SEQ ID NOs: 136-160, 256-257. In some embodiments, exemplary V_{H} are depicted in FIG. 24.

As applied to any of the antibody conjugates, in some embodiments, the light chain variable region (V_{L}) comprises an amino acid sequence having at least 75%, 80%, 85%, 90%, 95%, or 100% sequence identity to any sequence according to SEQ ID NOs: 161-187, 258-259. In some embodiments, the light chain variable region is selected from the group consisting of SEQ ID NOs: 161-187, 258-259. In some embodiments, exemplary V_{L} are depicted in FIG. 25.

As applied to any of the antibody conjugates, in some embodiments, the anti-Gal3 antibody or binding fragment thereof comprises: 1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161; 2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162; 3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163; 4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164; 5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165; 6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166; 7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167; 8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168; 9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169; 10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170; 11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171; 12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172; 13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173; 14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175; 16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176; 17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177; 18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178; 19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179; 20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180; 21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181; 22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182; 23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183; 24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184; 25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185; 26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186; 27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187; 28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258; 29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259; 30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811. In some embodiments, exemplary combinations of heavy chain variable region CDRs are depicted in FIG. 28. In some embodiments, exemplary combinations of light chain variable region CDRs are depicted in FIG. 29.

As applied to any of the antibody conjugates, in some embodiments, the anti-Gal3 antibody or binding fragment thereof comprises: 1) the heavy chain variable region of SEQ ID NO: 136 and the light chain variable region of SEQ ID NO: 161; 2) the heavy chain variable region of SEQ ID NO: 137 and the light chain variable region of SEQ ID NO: 162; 3) the heavy chain variable region of SEQ ID NO: 138 and the light chain variable region of SEQ ID NO: 163; 4) the heavy chain variable region of SEQ ID NO: 139 and the light chain variable region of SEQ ID NO: 164; 5) the heavy chain variable region of SEQ ID NO: 140 and the light chain variable region of SEQ ID NO: 165; 6) the heavy chain variable region of SEQ ID NO: 141 and the light chain variable region of SEQ ID NO: 166; 7) the heavy chain variable region of SEQ ID NO: 142 and the light chain variable region of SEQ ID NO: 167; 8) the heavy chain variable region of SEQ ID NO: 143 and the light chain variable region of SEQ ID NO: 168; 9) the heavy chain variable region of SEQ ID NO: 144 and the light chain variable region of SEQ ID NO: 169; 10) the heavy chain variable region of SEQ ID NO: 145 and the light chain variable region of SEQ ID NO: 170; 11) the heavy chain variable region of SEQ ID NO: 139 and the light chain variable region of SEQ ID NO: 171; 12) the heavy chain variable region of SEQ ID NO: 146 and the light chain variable region of SEQ ID NO: 172; 13) the heavy chain variable region of SEQ ID NO: 147 and the light chain variable region of SEQ ID NO: 173; 14) the heavy chain variable region of SEQ ID NO: 148 and the light chain variable region of SEQ ID NO: 174; 15) the heavy chain variable region of SEQ ID NO: 149 and the light chain variable region of SEQ ID NO: 175; 16) the heavy chain variable region of SEQ ID NO: 150 and the light chain variable region of SEQ ID NO: 176; 17) the heavy chain variable region of SEQ ID NO: 151 and the light chain variable region of SEQ ID NO: 177; 18) the heavy chain variable region of SEQ ID NO: 152 and the light chain variable region of SEQ ID NO: 178; 19) the heavy chain variable region of SEQ ID NO: 153 and the light chain variable region of SEQ ID NO: 179; 20) the heavy chain variable region of SEQ ID NO: 154 and the light chain variable region of SEQ ID NO: 180; 21) the heavy chain variable region of SEQ ID NO: 155 and the light chain variable region of SEQ ID NO: 181; 22) the heavy chain variable region of SEQ ID NO: 156 and the light chain variable region of SEQ ID NO: 182; 23) the heavy chain variable region of SEQ ID NO: 157 and the light chain variable region of SEQ ID NO: 183; 24) the heavy chain variable region of SEQ ID NO: 155 and the light chain variable region of SEQ ID NO: 184; 25) the heavy chain variable region of SEQ ID NO: 158 and the light chain variable region of SEQ ID NO: 185; 26) the heavy chain variable region of SEQ ID NO: 159 and the light chain variable region of SEQ ID NO: 186; 27) the heavy chain variable region of SEQ ID NO: 160 and the light chain variable region of SEQ ID NO: 187; 28) the heavy chain variable region of SEQ ID NO: 256 and the light chain variable region of SEQ ID NO: 258; 29) the heavy chain variable region of SEQ ID NO: 257 and the light chain variable region of SEQ ID NO: 259; 30) the heavy chain variable region of SEQ ID NO: 756 and the light chain variable region of SEQ ID NO: 784; 31) the heavy chain variable region of SEQ ID NO: 757 and the light chain variable region of SEQ ID NO: 785; 32) the heavy chain variable region of SEQ ID NO: 758 and the light chain variable region of SEQ ID NO: 786; 33) the heavy chain variable region of SEQ ID NO: 759 and the light chain variable region of SEQ ID NO: 787; 34) the heavy chain variable region of SEQ ID NO: 760 and the light chain variable region of SEQ ID NO: 788; 35) the heavy chain variable region of SEQ ID NO: 761 and the light chain variable region of SEQ ID NO: 789; 36) the heavy chain variable region of SEQ ID NO: 762 and the light chain variable region of SEQ ID NO: 790; 37) the heavy chain variable region of SEQ ID NO: 763 and the light chain variable region of SEQ ID NO: 791; 38) the heavy chain variable region of SEQ ID NO: 764 and the light chain variable region of SEQ ID NO: 792; 39) the heavy chain variable region of SEQ ID NO: 765 and the light chain variable region of SEQ ID NO: 793; 40) the heavy chain variable region of SEQ ID NO: 766 and the light chain variable region of SEQ ID NO: 794; 41) the heavy chain variable region of SEQ ID NO: 767 and the light chain variable region of SEQ ID NO: 795; 42) the heavy chain variable region of SEQ ID NO: 768 and the light chain variable region of SEQ ID NO: 796; 43) the heavy chain variable region of SEQ ID NO: 769 and the light chain variable region of SEQ ID NO: 797; 44) the heavy chain variable region of SEQ ID NO: 770 and the light chain variable region of SEQ ID NO: 798; 45) the heavy chain variable region of SEQ ID NO: 771 and the light chain variable region of SEQ ID NO: 799; 46) the heavy chain variable region of SEQ ID NO: 772 and the light chain variable region of SEQ ID NO: 800; 47) the heavy chain variable region of SEQ ID NO: 773 and the light chain variable region of SEQ ID NO: 801; 48) the heavy chain variable region of SEQ ID NO: 774 and the light chain variable region of SEQ ID NO: 802; 49) the heavy chain variable region of SEQ ID NO: 775 and the light chain variable region of SEQ ID NO: 803; 50) the heavy chain variable region of SEQ ID NO: 776 and the light chain variable region of SEQ ID NO: 804; 51) the heavy chain variable region of SEQ ID NO: 777 and the light chain variable region of SEQ ID NO: 805; 52) the heavy chain variable region of SEQ ID NO: 778 and the light chain variable region of SEQ ID NO: 806; 53) the heavy chain variable region of SEQ ID NO: 779 and the light chain variable region of SEQ ID NO: 807; 54) the heavy chain variable region of SEQ ID NO: 780 and the light chain variable region of SEQ ID NO: 808; 55) the heavy chain variable region of SEQ ID NO: 781 and the light chain variable region of SEQ ID NO: 809; 56) the heavy chain variable region of SEQ ID NO: 782 and the light chain variable region of SEQ ID NO: 810; or 57) the heavy chain variable region of SEQ ID NO: 783 and the light chain variable region of SEQ ID NO: 811.

As applied to any of the antibody conjugates, in some embodiments, the anti-Gal3 antibody or binding fragment thereof comprises the heavy chain (HC) sequence of any one of SEQ ID NOs: 188-216. In some embodiments, exemplary HC sequences are depicted in FIG. 26.

As applied to any of the antibody conjugates, in some embodiments, the anti-Gal3 antibody or binding fragment thereof comprises the light chain (LC) sequence of any one of SEQ ID NOs: 217-243. In some embodiments, exemplary LC sequences are depicted in FIG. 27.

As applied to any of the antibody conjugates, in some embodiments, the anti-Gal3 antibody or binding fragment thereof is selected from the group consisting of at least one of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. As applied to any of the antibody conjugates, in some embodiments, the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. In some embodiments, the anti-Gal3 antibody or binding fragment thereof is selected from the group consisting of at least one of TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof. In some embodiments, the anti-Gal3 antibody or binding fragment thereof disrupts an interaction between Gal3 and an antibody selected from 846.4D5, 15F10.2D6, F846C.1B2, and F846C.1H12. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32.

Also disclosed herein are multi-specific antibodies comprising a first binding domain that binds to Gal3 and a second binding domain that binds to a therapeutic target molecule located in the brain of a subject. In some embodiments, the second binding domain is not independently capable of crossing the blood-brain barrier or has low permeability across the blood-brain barrier without being conjugated to the anti-Gal3 antibody or binding fragment thereof. In some embodiments, the permeability of the second binding domain across the blood-brain barrier is less than 95%, 90%, 80%, 70%, 60%, 50%, 40%, 30%, 20%, 10%, 9%, 8%, 7%, 6%, 5%, 4%, 3%, 2%, or 1% of the permeability of the multi-specific antibody across the blood-brain barrier. In some embodiments, the first binding domain that binds to Gal3 belongs to bin 3, 8, 17, or 24. In some embodiments, the first binding domain that binds to Gal3 disrupts an interaction between Gal3 and an antibody that belongs to bin 3, 8, 17 or 24. In some embodiments, the first binding domain that binds to Gal3 competes with an antibody that belongs to bins 3, 8, 17 or 24 for binding to Gal3. In some embodiments, the first binding domain that binds to Gal3 is a binding domain of the anti-Gal3 antibody or binding fragment thereof of any one of the antibody conjugates of claims 109-133. In some embodiments, the first binding domain is a binding domain of any one of the anti-Gal3 antibodies or binding fragments thereof disclosed herein or a binding domain of any one of the antibody conjugates disclosed herein.

Also disclosed herein are pharmaceutical compositions comprising any one of the antibody conjugates or multi-specific antibodies disclosed herein and at least one pharmaceutically acceptable diluent, excipient, or carrier.

Also disclosed herein are methods of delivering a payload to a central nervous system of a subject in need thereof, comprising administering to the subject an antibody conjugate comprising an anti-Gal3 antibody or binding fragment thereof and a payload conjugated to the anti-Gal3 antibody or binding fragment thereof, wherein the antibody conjugate is able to cross the blood-brain barrier.

Also disclosed herein are methods of increasing the permeability of a payload across the blood-brain barrier of a subject in need thereof, comprising conjugating an anti-Gal3 antibody or binding fragment thereof to the payload to form an antibody conjugate. In some embodiments, the methods further comprise administering to the subject the antibody conjugate.

As applied to any of the methods comprising an anti-Gal3 antibody or binding fragment thereof conjugated to a payload, in some embodiments, the subject is a mammal, such as a mouse, rat, other rodent, cat, dog, rabbit, cow, horse, sheep, pig, goat, or human. In some embodiments, the payload does not normally cross the blood-brain barrier. In some embodiments, conjugating the payload to the anti-Gal3 antibody or binding fragment thereof increases the permeability of the payload across the blood-brain barrier by at least 5%, 10%, 20%, 30%, 40%, 50%, 60%, 70%, 80%, 90%, 100%, 200%, 300%, 400%, or 500%, or any increase within a range defined by any two of the aforementioned percentages, compared to the unconjugated payload. In some embodiments, the permeability of the payload across the blood-brain barrier is less than 95%, 90%, 80%, 70%, 60%, 50%, 40%, 30%, 20%, 10%, 9%, 8%, 7%, 6%, 5%, 4%, 3%, 2%, or 1% of the permeability of the antibody conjugate across the blood-brain barrier. In some embodiments, the payload, or the anti-Gal3 antibody or binding fragment thereof, or both, is used to treat a neurological disorder. In some embodiments, the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer (primary or secondary brain tumors), or any combination thereof. In some embodiments, the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, protein, enzyme, or any combination thereof. In some embodiments, the payload is the second antibody. In some embodiments, the second antibody is not independently capable of crossing the blood-brain barrier or has low permeability across the blood-brain barrier without being conjugated to the anti-Gal3 antibody or binding fragment thereof. In some embodiments, the subject is a mammal. In some embodiments, the subject is a human. In some embodiments, the antibody conjugate is administered enterally, orally, intranasally, parenterally, intracranially, subcutaneously, intramuscularly, intradermally, or intravenously, or any combination thereof.

As applied to any of the methods comprising an antibody conjugate, in some embodiments, the anti-Gal3 antibody or binding fragment thereof binds to one or more peptides of SEQ ID NOs: 3-26. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof belongs to bin 3, 8, 17, or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof disrupts an interaction between Gal3 and an antibody that belongs to bin 3, 8, 17 or 24. In some embodiments, the anti-Gal3 antibody or binding fragment thereof competes with an antibody that belongs to bins 3, 8, 17 or 24 for binding to Gal3.

As applied to any of the methods or uses comprising an antibody conjugate, in some embodiments, the anti-Gal3 antibody or binding fragment thereof comprises any one or more sequences (such as a V_{H}-CDR1, V_{H}-CDR2, V_{H}-CDR3, V_{L}-CDR1, V_{L}-CDR2, V_{L}-CDR3, heavy chain variable region, light chain variable region, heavy chain, or light chain sequence) provided throughout this disclosure. In some embodiments, the anti-Gal3 antibody or binding fragment thereof comprises any one or more sequences as shown in FIG. 18-32, including any one or more CDRs, heavy chain variable regions, light chain variable regions, heavy chains, light chains, combinations of CDRs, combinations of variable regions, or combinations of heavy chain and light chain described therein. In some embodiments, the anti-Gal3 antibody or binding fragment thereof comprises a peptide sequence having at least 80%, 85%, 90%, 95%, 99%, or 100% homology to the peptide sequence encoded by any one or more of the nucleic acid sequences as shown in FIG. 37-40, including any nucleic sequences encoding for a heavy chain variable region, light chain variable region, heavy chain, or light chain.

As applied to any of the methods comprising an antibody conjugate, in some embodiments, the anti-Gal3 antibody or binding fragment thereof comprises (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 27-44, 245-246, 588-615, the V_{H}-CDR2 comprises an amino acid sequence selected from SEQ ID NOs: 45-60, 247-248, 616-643, the V_{H}-CDR3 comprises an amino acid sequence selected from SEQ ID NOs: 61-81, 249-250, 644-671, the V_{L}-CDR1 comprises an amino acid sequence selected from SEQ ID NOs: 82-101, 251-252, 672-699, the V_{L}-CDR2 comprises an amino acid sequence selected from SEQ ID NOs: 102-116, 253, 700-727, and the V_{L}-CDR3 comprises an amino acid sequence selected from SEQ ID NOs: 117-135, 254-255, 728-755.

As applied to any of the methods comprising an antibody conjugate, in some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23.

As applied to any of the methods comprising an antibody conjugate, in some embodiments, the heavy chain variable region (V_{H}) comprises an amino acid sequence having at least 75%, 80%, 85%, 90%, 95%, or 100% sequence identity to any sequence according to SEQ ID NOs: 136-160, 256-257. In some embodiments, the heavy chain variable region is selected from the group consisting of SEQ ID NOs: 136-160, 256-257. In some embodiments, exemplary V_{H} are depicted in FIG. 24.

As applied to any of the methods comprising an antibody conjugate, in some embodiments, the light chain variable region (VL) comprises an amino acid sequence having at least 75%, 80%, 85%, 90%, 95%, or 100% sequence identity to any sequence according to SEQ ID NOs: 161-187, 258-259. In some embodiments, the light chain variable region is selected from the group consisting of SEQ ID NOs: 161-187, 258-259. In some embodiments, exemplary V_{L} are depicted in FIG. 25.

As applied to any of the methods comprising an antibody conjugate, in some embodiments, the anti-Gal3 antibody or binding fragment thereof comprises: 1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161; 2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162; 3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163; 4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164; 5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165; 6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166; 7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167; 8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168; 9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169; 10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170; 11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171; 12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172; 13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173; 14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175; 16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176; 17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177; 18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178; 19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179; 20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180; 21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181; 22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182; 23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183; 24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184; 25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185; 26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186; 27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187; 28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258; 29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259; 30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811. In some embodiments, exemplary combinations of heavy chain variable region CDRs are depicted in FIG. 28. In some embodiments, exemplary combinations of light chain variable region CDRs are depicted in FIG. 29.

As applied to any of the methods comprising an antibody conjugate, in some embodiments, the anti-Gal3 antibody or binding fragment thereof comprises: 1) the heavy chain variable region of SEQ ID NO: 136 and the light chain variable region of SEQ ID NO: 161; 2) the heavy chain variable region of SEQ ID NO: 137 and the light chain variable region of SEQ ID NO: 162; 3) the heavy chain variable region of SEQ ID NO: 138 and the light chain variable region of SEQ ID NO: 163; 4) the heavy chain variable region of SEQ ID NO: 139 and the light chain variable region of SEQ ID NO: 164; 5) the heavy chain variable region of SEQ ID NO: 140 and the light chain variable region of SEQ ID NO: 165; 6) the heavy chain variable region of SEQ ID NO: 141 and the light chain variable region of SEQ ID NO: 166; 7) the heavy chain variable region of SEQ ID NO: 142 and the light chain variable region of SEQ ID NO: 167; 8) the heavy chain variable region of SEQ ID NO: 143 and the light chain variable region of SEQ ID NO: 168; 9) the heavy chain variable region of SEQ ID NO: 144 and the light chain variable region of SEQ ID NO: 169; 10) the heavy chain variable region of SEQ ID NO: 145 and the light chain variable region of SEQ ID NO: 170; 11) the heavy chain variable region of SEQ ID NO: 139 and the light chain variable region of SEQ ID NO: 171; 12) the heavy chain variable region of SEQ ID NO: 146 and the light chain variable region of SEQ ID NO: 172; 13) the heavy chain variable region of SEQ ID NO: 147 and the light chain variable region of SEQ ID NO: 173; 14) the heavy chain variable region of SEQ ID NO: 148 and the light chain variable region of SEQ ID NO: 174; 15) the heavy chain variable region of SEQ ID NO: 149 and the light chain variable region of SEQ ID NO: 175; 16) the heavy chain variable region of SEQ ID NO: 150 and the light chain variable region of SEQ ID NO: 176; 17) the heavy chain variable region of SEQ ID NO: 151 and the light chain variable region of SEQ ID NO: 177; 18) the heavy chain variable region of SEQ ID NO: 152 and the light chain variable region of SEQ ID NO: 178; 19) the heavy chain variable region of SEQ ID NO: 153 and the light chain variable region of SEQ ID NO: 179; 20) the heavy chain variable region of SEQ ID NO: 154 and the light chain variable region of SEQ ID NO: 180; 21) the heavy chain variable region of SEQ ID NO: 155 and the light chain variable region of SEQ ID NO: 181; 22) the heavy chain variable region of SEQ ID NO: 156 and the light chain variable region of SEQ ID NO: 182; 23) the heavy chain variable region of SEQ ID NO: 157 and the light chain variable region of SEQ ID NO: 183; 24) the heavy chain variable region of SEQ ID NO: 155 and the light chain variable region of SEQ ID NO: 184; 25) the heavy chain variable region of SEQ ID NO: 158 and the light chain variable region of SEQ ID NO: 185; 26) the heavy chain variable region of SEQ ID NO: 159 and the light chain variable region of SEQ ID NO: 186; 27) the heavy chain variable region of SEQ ID NO: 160 and the light chain variable region of SEQ ID NO: 187; 28) the heavy chain variable region of SEQ ID NO: 256 and the light chain variable region of SEQ ID NO: 258; 29) the heavy chain variable region of SEQ ID NO: 257 and the light chain variable region of SEQ ID NO: 259; 30) the heavy chain variable region of SEQ ID NO: 756 and the light chain variable region of SEQ ID NO: 784; 31) the heavy chain variable region of SEQ ID NO: 757 and the light chain variable region of SEQ ID NO: 785; 32) the heavy chain variable region of SEQ ID NO: 758 and the light chain variable region of SEQ ID NO: 786; 33) the heavy chain variable region of SEQ ID NO: 759 and the light chain variable region of SEQ ID NO: 787; 34) the heavy chain variable region of SEQ ID NO: 760 and the light chain variable region of SEQ ID NO: 788; 35) the heavy chain variable region of SEQ ID NO: 761 and the light chain variable region of SEQ ID NO: 789; 36) the heavy chain variable region of SEQ ID NO: 762 and the light chain variable region of SEQ ID NO: 790; 37) the heavy chain variable region of SEQ ID NO: 763 and the light chain variable region of SEQ ID NO: 791; 38) the heavy chain variable region of SEQ ID NO: 764 and the light chain variable region of SEQ ID NO: 792; 39) the heavy chain variable region of SEQ ID NO: 765 and the light chain variable region of SEQ ID NO: 793; 40) the heavy chain variable region of SEQ ID NO: 766 and the light chain variable region of SEQ ID NO: 794; 41) the heavy chain variable region of SEQ ID NO: 767 and the light chain variable region of SEQ ID NO: 795; 42) the heavy chain variable region of SEQ ID NO: 768 and the light chain variable region of SEQ ID NO: 796; 43) the heavy chain variable region of SEQ ID NO: 769 and the light chain variable region of SEQ ID NO: 797; 44) the heavy chain variable region of SEQ ID NO: 770 and the light chain variable region of SEQ ID NO: 798; 45) the heavy chain variable region of SEQ ID NO: 771 and the light chain variable region of SEQ ID NO: 799; 46) the heavy chain variable region of SEQ ID NO: 772 and the light chain variable region of SEQ ID NO: 800; 47) the heavy chain variable region of SEQ ID NO: 773 and the light chain variable region of SEQ ID NO: 801; 48) the heavy chain variable region of SEQ ID NO: 774 and the light chain variable region of SEQ ID NO: 802; 49) the heavy chain variable region of SEQ ID NO: 775 and the light chain variable region of SEQ ID NO: 803; 50) the heavy chain variable region of SEQ ID NO: 776 and the light chain variable region of SEQ ID NO: 804; 51) the heavy chain variable region of SEQ ID NO: 777 and the light chain variable region of SEQ ID NO: 805; 52) the heavy chain variable region of SEQ ID NO: 778 and the light chain variable region of SEQ ID NO: 806; 53) the heavy chain variable region of SEQ ID NO: 779 and the light chain variable region of SEQ ID NO: 807; 54) the heavy chain variable region of SEQ ID NO: 780 and the light chain variable region of SEQ ID NO: 808; 55) the heavy chain variable region of SEQ ID NO: 781 and the light chain variable region of SEQ ID NO: 809; 56) the heavy chain variable region of SEQ ID NO: 782 and the light chain variable region of SEQ ID NO: 810; or 57) the heavy chain variable region of SEQ ID NO: 783 and the light chain variable region of SEQ ID NO: 811.

As applied to any of the methods comprising an antibody conjugate, in some embodiments, the anti-Gal3 antibody or binding fragment thereof comprises the heavy chain (HC) sequence of any one of SEQ ID NOs: 188-216. In some embodiments, exemplary HC sequences are depicted in FIG. 26.

As applied to any of the methods comprising an antibody conjugate, in some embodiments, the anti-Gal3 antibody or binding fragment thereof comprises the light chain (LC) sequence of any one of SEQ ID NOs: 217-243. In some embodiments, exemplary LC sequences are depicted in FIG. 27.

As applied to any of the methods comprising an antibody conjugate, in some embodiments, the anti-Gal3 antibody or binding fragment thereof is selected from at least one of the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. As applied to any of the methods comprising an antibody conjugate, in some embodiments, the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. In some embodiments, the anti-Gal3 antibody or binding fragment thereof is selected from at least one of the group consisting of TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof. In some embodiments, the anti-Gal3 antibody or binding fragment thereof disrupts an interaction between Gal3 and an antibody selected from 846.4D5, 15F10.2D6, F846C.1B2, and F846C.1H12. In some embodiments, the anti-Gal3 antibody or binding fragment thereof competes with an antibody that belongs to bins 3, 8, 17 or 24 for binding to Gal3. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32.

### Methods of Use—Disruption of Cell Surface Markers

Galectin-3 (Gal3) is known to play an important role in cell proliferation, adhesion, differentiation, angiogenesis, and apoptosis. This activity is, at least in part, due to immunomodulatory properties and binding affinity towards other immune regulatory proteins, signaling proteins, and other cell surface markers. As disclosed herein, Gal3 is shown to directly bind to TGF-beta receptors, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof. Therefore, Gal3 may contribute to pro-inflammatory or anti-inflammatory reactions and inflammation-related disorders. Gal3 functions by distinct N-terminal and C-terminal domains. The N-terminal domain (amino acids 1-111) comprise a tandem repeat domain (TRD, amino acids 36-109) and is largely responsible for oligomerization of Gal3. The C-terminal domain (amino acids 112-250) comprise a carbohydrate-recognition-binding domain (CRD), which binds to β-galactosides.

In some embodiments are disclosed methods of using any one or more of the anti-Gal3 antibodies or binding fragments thereof to block or disrupt an interaction between a TGF-beta receptor, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof, either in vitro or in vivo.

In some embodiments, methods are directed towards disrupting an interaction between Gal3 and a TGF-b receptor. In some embodiments, the methods comprise contacting an interaction between Gal and the TGF-b receptor with an antibody or binding fragment thereof that selectively binds to Gal3 and disrupts the interaction between Gal3 and the TGF-b receptor (e.g. any one of the anti-Gal3 antibodies or binding fragments thereof disclosed herein). In some embodiments, the Gal3 is expressed by a cell. In some embodiments, the Gal3 is secreted by a cell. In some embodiments, the TGF-b receptor is expressed by a cell.

In some embodiments, methods are directed to treating fibrosis in a subject in need thereof. In some embodiments, the methods comprise administering to the subject an antibody or binding fragment thereof that selectively binds to Gal3 and disrupts the interaction between Gal3 and the TGF-b receptor (e.g. any one of the anti-Gal3 antibodies or binding fragments thereof disclosed herein), thereby treating the fibrosis in the subject. In some embodiments, the fibrosis is liver fibrosis, kidney fibrosis, cardiac fibrosis, arterial fibrosis, venous thrombosis, or pulmonary fibrosis.

In some embodiments, methods are directed to treating non-alcoholic fatty liver disease (NAFLD) or non-alcoholic steatohepatitis (NASH) in a subject in need thereof. In some embodiments, the methods comprise administering to the subject an antibody or binding fragment thereof that selectively binds to Gal3 and disrupts the interaction between Gal3 and the TGF-b receptor (e.g. any one of the anti-Gal3 antibodies or binding fragments thereof disclosed herein), thereby treating the NAFLD or NASH in the subject.

In some embodiments, methods are directed to treating an immune-related disorder in a subject in need thereof. In some embodiments, the methods comprise administering to the subject an antibody or binding fragment thereof that selectively binds to Gal3 and disrupts the interaction between Gal3 and the TGF-b receptor (e.g. any one of the anti-Gal3 antibodies or binding fragments thereof disclosed herein), thereby treating the immune-related disorder in the subject. In some embodiments, the immune-related disorder is sepsis, atopic dermatitis, or psoriasis. In some embodiments, the immune-related disorder is cancer. In some embodiments, the antibody or binding fragment thereof is administered as a supplement to PD1/PDL1 blockade therapies and/or a CTLA4 blockade therapy. In some embodiments, the PD1/PDL1 blockade therapies comprise pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and/or BMS-986189. In some embodiments, the CTLA4 blockade therapy comprises ipilimumab and/or tremilimumab.

As applied to any of the methods disclosed herein involving the disruption of an interaction between Gal3 and a TGF-b receptor, the TGF-b receptor is TGF-b receptor 1, TGF-b receptor 2, or TGF-b receptor 3.

Also disclosed herein in some embodiments are methods of disrupting an interaction between Gal3 and a tumor cell surface marker. In some embodiments, the methods comprise contacting the tumor cell surface marker with an anti-Gal3 antibody or binding fragment thereof specific for the N-terminal domain of Gal3, N-terminus of Gal3, or the TRD of Gal3. In some embodiments, the tumor cell surface marker is selected from the group consisting of VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, and FGFR4.

Also disclosed herein in some embodiments are methods of disrupting an interaction between Gal3 and a TGF-b receptor, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof. In some embodiments, the method comprises contacting an interaction site between Gal3 and the TGF-b receptor, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof with an antibody or binding fragment thereof that selectively binds to Gal3 and disrupts the interaction between Gal3 and the TGF-b receptor, VEGFR1, VEGFR2, VEGFR3, EGFR, PDGFRa, PDGFRb, ErbB2, HGFR, TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof.

Also disclosed herein in some embodiments are methods of treating a cancer in a subject in need thereof. In some embodiments, the methods comprise administering to the subject an anti-Gal3 antibody or binding fragment thereof specific for the N-terminal domain of Gal3, N-terminus of Gal3, or the TRD of Gal3. In some embodiments, the anti-Gal3 antibody or binding fragment thereof disrupts an interaction between Gal3 and a tumor cell surface marker, and the tumor cell surface marker is selected from the group consisting of VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, and FGFR4. In some embodiments, the cancer is brain cancer, breast cancer, colorectal cancer, kidney cancer, liver cancer, lung cancer, pancreatic cancer, bladder cancer, stomach cancer, or a hematological malignancy. In some embodiments, the methods further comprise administering a standard of care treatment, and the anti-Gal3 antibody or binding fragment thereof is used as a supplement to the standard of care treatment. In some embodiments, the standard of care treatment comprises surgery, radiation, chemotherapy, targeted therapy, immunotherapy, a PD1/PDL1 blockade therapy, a CTLA4 blockade therapy, temozolomide, or any combination thereof.

In some embodiments, the interaction between Gal3 and a cell surface marker or tumor surface marker can be reduced to less than 80%, less than 75%, less than 70%, less than 60%, less than 59%, less than 50%, less than 40%, less than 34%, less than 30%, less than 20%, less than 14%, less than 10%, less than 7%, less than 5%, less than 4%, or less than 1%.

In some embodiments, the antibody or binding fragment thereof binds to Gal3 with a dissociation constant (KD) of less than 1 nM, less than 1.2 nM, less than 2 nM, less than 5 nM, less than 10 nM, less than 13.5 nM, less than 15 nM, less than 20 nM, less than 25 nM, or less than 30 nM.

In some embodiments, the interaction between Gal3 and the TGF-beta receptor, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4 can be reduced to less than 80%, less than 75%, less than 70%, less than 60%, less than 59%, less than 50%, less than 40%, less than 34%, less than 30%, less than 20%, less than 14%, less than 10%, less than 7%, less than 5%, less than 4%, or less than 1%.

As applied to any of the methods disclosed herein, in some embodiments, the antibody or binding fragment thereof is formulated for systemic administration. In some embodiments, the antibody or binding fragment thereof is formulated for parenteral administration. In some embodiments, the anti-Gal3 antibody or binding fragment thereof is administered enterally, orally, intranasally, parenterally, intracranially, subcutaneously, intramuscularly, intradermally, or intravenously, or any combination thereof. In some embodiments, the subject is a mammal. In some embodiments, the subject is a human.

As applied to any of the methods disclosed herein, in some embodiments, the anti-Gal3 antibody or binding fragment thereof comprises any one or more sequences (such as a V_{H}-CDR1, V_{H}-CDR2, V_{H}-CDR3, V_{L}-CDR1, V_{L}-CDR2, V_{L}-CDR3, heavy chain variable region, light chain variable region, heavy chain, or light chain sequence) provided throughout this disclosure. In some embodiments, the anti-Gal3 antibody or binding fragment thereof comprises any one or more sequences as shown in FIG. 18-32, including any one or more CDRs, heavy chain variable regions, light chain variable regions, heavy chains, light chains, combinations of CDRs, combinations of variable regions, or combinations of heavy chain and light chain described therein. In some embodiments, the anti-Gal3 antibody or binding fragment thereof comprises a peptide sequence having at least 80%, 85%, 90%, 95%, 99%, or 100% homology to the peptide sequence encoded by any one or more of the nucleic acid sequences as shown in FIG. 37-40, including any nucleic sequences encoding for a heavy chain variable region, light chain variable region, heavy chain, or light chain.

As applied to any of the methods disclosed herein, in some embodiments, the anti-Gal3 antibody or binding fragment thereof binds to one or more peptides of SEQ ID NOs: 3-26. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof binds to an epitope present within a region of Gal3 defined by Peptide 1 (SEQ ID NO: 3), Peptide 4 (SEQ ID NO: 6), Peptide 6 (SEQ ID NO: 8), Peptide 7 (SEQ ID NO: 9), or any combination thereof. In some embodiments, the antibody or binding fragment thereof binds to an epitope of Gal3 comprising an amino acid sequence of GxYPG, wherein X is alanine, glycine, or valine.

As applied to any of the methods disclosed herein, in some embodiments, the antibody or binding fragment comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 60%, at least 70%, at least 80%, at least 90%, or 100% sequence identity to any amino acid sequence according to SEQ ID NOs: 27-36, 397-399, 588-615; the V_{H}-CDR2 comprises an amino acid sequence having at least 60%, at least 70%, at least 80%, at least 90%, or 100% sequence identity to any amino acid sequence according to SEQ ID NOs: 45-54, 400-406, 616-643; the V_{H}-CDR3 comprises an amino acid sequence having at least 60%, at least 70%, at least 80%, at least 90%, or 100% sequence identity to any amino acid sequence according to SEQ ID NOs: 61-69, 71, 408-416, 644-671; the V_{L}-CDR1 comprises an amino acid sequence having at least 60%, at least 70%, at least 80%, at least 90%, or 100% sequence identity to any amino acid sequence according to SEQ ID NOs: 82-92, 417-426, 672-699; the V_{L}-CDR2 comprises an amino acid sequence having at least 60%, at least 70%, at least 80%, at least 90%, or 100% sequence identity to any amino acid sequence according to SEQ ID NOs: 102-111, 427-428, 700-727; and the V_{L}-CDR3 comprises an amino acid sequence having at least 60%, at least 70%, at least 80%, at least 90%, or 100% sequence identity to any amino acid sequence according to SEQ ID NOs: 117-127, 429-434, 728-755.

As applied to any of the methods disclosed herein, in some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23. In some embodiments, any of the methods disclosed herein involving an anti-Gal3 antibody or binding fragment can be performed with an antigen binding molecule that binds to Gal3.

As applied to any of the methods disclosed herein, in some embodiments, the heavy chain variable region (V_{H}) comprises an amino acid sequence having at least 75%, 80%, 85%, 90%, 95%, or 100% sequence identity to any sequence according to SEQ ID NOs: 136-148, 436, 438-450, 756-783. In some embodiments, the heavy chain variable region is selected from the group consisting of SEQ ID NOs: 136-148, 436, 438-450, 756-783. In some embodiments, exemplary V_{H} are depicted in FIG. 24. In some embodiments, any of the methods disclosed herein involving an anti-Gal3 antibody or binding fragment can be performed with an antigen binding molecule that binds to Gal3.

As applied to any of the methods disclosed herein, in some embodiments, the light chain variable region (V_{L}) comprises an amino acid sequence having at least 75%, 80%, 85%, 90%, 95%, or 100% sequence identity to any sequence according to SEQ ID NOs: 161-172, 174, 451, 453-464, 784-811. In some embodiments, the light chain variable region is selected from the group consisting of SEQ ID NOs: 161-172, 174, 451, 453-464, 784-811. In some embodiments, exemplary V_{L} are depicted in FIG. 25. In some embodiments, any of the methods disclosed herein involving an anti-Gal3 antibody or binding fragment can be performed with an antigen binding molecule that binds to Gal3.

As applied to any of the methods disclosed herein, in some embodiments, the antibody or binding fragment comprises: 1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161; 2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162; 3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163; 4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164; 5) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171; 6) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165; 7) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166; 8) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167; 9) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168; 10) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169; 11) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170; 12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, VL-CDR3 of the V_{L}-CDR1, V_{L}-CDR2, V_{L}-CDR3 within SEQ ID NO: 172; 13) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 14) the V_{H}-CDR1, V_{H}-CDR2, V_{H}-CDR3 of the V_{H}-CDR1, V_{H}-CDR2, V_{H}-CDR3 within SEQ ID NO: 436 and the V_{L}-CDR1, V_{L}-CDR2, V_{L}-CDR3 of the V_{L}-CDR1, V_{L}-CDR2, V_{L}-CDR3 within SEQ ID NO: 451; 15) the V_{H}-CDR1, V_{H}-CDR2, V_{H}-CDR3 of the V_{H}-CDR1, V_{H}-CDR2, V_{H}-CDR3 within SEQ ID NO: 438 and the V_{L}-CDR1, V_{L}-CDR2, V_{L}-CDR3 of the V_{L}-CDR1, V_{L}-CDR2, V_{L}-CDR3 within SEQ ID NO: 453; 16) the V_{H}-CDR1, V_{H}-CDR2, V_{H}-CDR3 of the V_{H}-CDR1, V_{H}-CDR2, V_{H}-CDR3 within SEQ ID NO: 439 and the V_{L}-CDR1, V_{L}-CDR2, V_{L}-CDR3 of the V_{L}-CDR1, V_{L}-CDR2, V_{L}-CDR3 within SEQ ID NO: 162; 17) the V_{H}-CDR1, V_{H}-CDR2, V_{H}-CDR3 of the V_{H}-CDR1, V_{H}-CDR2, V_{H}-CDR3 within SEQ ID NO: 440 and the V_{L}-CDR1, V_{L}-CDR2, V_{L}-CDR3 of the V_{L}-CDR1, V_{L}-CDR2, V_{L}-CDR3 within SEQ ID NO: 454; 18) the V_{H}-CDR1, V_{H}-CDR2, V_{H}-CDR3 of the V_{H}-CDR1, V_{H}-CDR2, V_{H}-CDR3 within SEQ ID NO: 441 and the V_{L}-CDR1, V_{L}-CDR2, V_{L}-CDR3 of the V_{L}-CDR1, V_{L}-CDR2, V_{L}-CDR3 within SEQ ID NO: 455; 19) the V_{H}-CDR1, V_{H}-CDR2, V_{H}-CDR3 of the V_{H}-CDR1, V_{H}-CDR2, V_{H}-CDR3 within SEQ ID NO: 442 and the V_{L}-CDR1, V_{L}-CDR2, V_{L}-CDR3 of the V_{L}-CDR1, V_{L}-CDR2, V_{L}-CDR3 within SEQ ID NO: 456; 20) the V_{H}-CDR1, V_{H}-CDR2, V_{H}-CDR3 of the V_{H}-CDR1, V_{H}-CDR2, V_{H}-CDR3 within SEQ ID NO: 443 and the V_{L}-CDR1, V_{L}-CDR2, V_{L}-CDR3 of the V_{L}-CDR1, V_{L}-CDR2, V_{L}-CDR3 within SEQ ID NO: 457; 21) the V_{H}-CDR1, V_{H}-CDR2, V_{H}-CDR3 of the V_{H}-CDR1, V_{H}-CDR2, V_{H}-CDR3 within SEQ ID NO: 444 and the V_{L}-CDR1, V_{L}-CDR2, V_{L}-CDR3 of the V_{L}-CDR1, V_{L}-CDR2, V_{L}-CDR3 within SEQ ID NO: 458; 22) the V_{H}-CDR1, V_{H}-CDR2, V_{H}-CDR3 of the V_{H}-CDR1, V_{H}-CDR2, V_{H}-CDR3 within SEQ ID NO: 445 and the V_{L}-CDR1, V_{L}-CDR2, V_{L}-CDR3 of the V_{L}-CDR1, V_{L}-CDR2, V_{L}-CDR3 within SEQ ID NO: 459; 23) the V_{H}-CDR1, V_{H}-CDR2, V_{H}-CDR3 of the V_{H}-CDR1, V_{H}-CDR2, V_{H}-CDR3 within SEQ ID NO: 446 and the V_{L}-CDR1, V_{L}-CDR2, V_{L}-CDR3 of the V_{L}-CDR1, V_{L}-CDR2, V_{L}-CDR3 within SEQ ID NO: 460; 24) the V_{H}-CDR1, V_{H}-CDR2, V_{H}-CDR3 of the V_{H}-CDR1, V_{H}-CDR2, V_{H}-CDR3 within SEQ ID NO: 447 and the V_{L}-CDR1, V_{L}-CDR2, V_{L}-CDR3 of the V_{L}-CDR1, V_{L}-CDR2, V_{L}-CDR3 within SEQ ID NO: 461; 25) the V_{H}-CDR1, V_{H}-CDR2, V_{H}-CDR3 of the V_{H}-CDR1, V_{H}-CDR2, V_{H}-CDR3 within SEQ ID NO: 448 and the V_{L}-CDR1, V_{L}-CDR2, V_{L}-CDR3 of the V_{L}-CDR1, V_{L}-CDR2, V_{L}-CDR3 within SEQ ID NO: 462; 26) the V_{H}-CDR1, V_{H}-CDR2, V_{H}-CDR3 of the V_{H}-CDR1, V_{H}-CDR2, V_{H}-CDR3 within SEQ ID NO: 449 and the V_{L}-CDR1, V_{L}-CDR2, V_{L}-CDR3 of the V_{L}-CDR1, V_{L}-CDR2, V_{L}-CDR3 within SEQ ID NO: 463; 27) the V_{H}-CDR1, V_{H}-CDR2, V_{H}-CDR3 of the V_{H}-CDR1, V_{H}-CDR2, V_{H}-CDR3 within SEQ ID NO: 450 and the V_{L}-CDR1, V_{L}-CDR2, V_{L}-CDR3 of the V_{L}-CDR1, V_{L}-CDR2, V_{L}-CDR3 within SEQ ID NO: 464; 28) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 29) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 30) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 31) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 32) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 33) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 34) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 35) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 36) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 37) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 38) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 39) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 40) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 41) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 42) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 43) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 44) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 45) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 46) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 47) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 48) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 49) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 50) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 51) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 52) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 53) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 54) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 55) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

As applied to any of the methods disclosed herein, in some embodiments, the antibody or binding fragment comprises: 1) the heavy chain variable region of SEQ ID NO: 136 and the light chain variable region of SEQ ID NO: 161; 2) the heavy chain variable region of SEQ ID NO: 137 and the light chain variable region of SEQ ID NO: 162; 3) the heavy chain variable region of SEQ ID NO: 138 and the light chain variable region of SEQ ID NO: 163; 4) the heavy chain variable region of SEQ ID NO: 139 and the light chain variable region of SEQ ID NO: 164; 5) the heavy chain variable region of SEQ ID NO: 139 and the light chain variable region of SEQ ID NO: 171; 6) the heavy chain variable region of SEQ ID NO: 140 and the light chain variable region of SEQ ID NO: 165; 7) the heavy chain variable region of SEQ ID NO: 141 and the light chain variable region of SEQ ID NO: 166; 8) the heavy chain variable region of SEQ ID NO: 142 and the light chain variable region of SEQ ID NO: 167; 9) the heavy chain variable region of SEQ ID NO: 143 and the light chain variable region of SEQ ID NO: 168; 10) the heavy chain variable region of SEQ ID NO: 144 and the light chain variable region of SEQ ID NO: 169; 11) the heavy chain variable region of SEQ ID NO: 145 and the light chain variable region of SEQ ID NO: 170; 12) the heavy chain variable region of SEQ ID NO: 146 and the light chain variable region of SEQ ID NO: 172; 13) the heavy chain variable region of SEQ ID NO: 148 and the light chain variable region of SEQ ID NO: 174; 14) the heavy chain variable region of SEQ ID NO: 436 and the light chain variable region of SEQ ID NO: 451; 15) the heavy chain variable region of SEQ ID NO: 438 and the light chain variable region of SEQ ID NO: 453; 16) the heavy chain variable region of SEQ ID NO: 439 and the light chain variable region of SEQ ID NO: 162; 17) the heavy chain variable region of SEQ ID NO: 440 and the light chain variable region of SEQ ID NO: 454; 18) the heavy chain variable region of SEQ ID NO: 441 and the light chain variable region of SEQ ID NO: 455; 19) the heavy chain variable region of SEQ ID NO: 442 and the light chain variable region of SEQ ID NO: 456; 20) the heavy chain variable region of SEQ ID NO: 443 and the light chain variable region of SEQ ID NO: 457; 21) the heavy chain variable region of SEQ ID NO: 444 and the light chain variable region of SEQ ID NO: 458; 22) the heavy chain variable region of SEQ ID NO: 445 and the light chain variable region of SEQ ID NO: 459; 23) the heavy chain variable region of SEQ ID NO: 446 and the light chain variable region of SEQ ID NO: 460; 24) the heavy chain variable region of SEQ ID NO: 447 and the light chain variable region of SEQ ID NO: 461; 25) the heavy chain variable region of SEQ ID NO: 448 and the light chain variable region of SEQ ID NO: 462; 26) the heavy chain variable region of SEQ ID NO: 449 and the light chain variable region of SEQ ID NO: 463; or 27) the heavy chain variable region of SEQ ID NO: 450 and the light chain variable region of SEQ ID NO: 464; 28) the heavy chain variable region of SEQ ID NO: 756 and the light chain variable region of SEQ ID NO: 784; 29) the heavy chain variable region of SEQ ID NO: 757 and the light chain variable region of SEQ ID NO: 785; 30) the heavy chain variable region of SEQ ID NO: 758 and the light chain variable region of SEQ ID NO: 786; 31) the heavy chain variable region of SEQ ID NO: 759 and the light chain variable region of SEQ ID NO: 787; 32) the heavy chain variable region of SEQ ID NO: 760 and the light chain variable region of SEQ ID NO: 788; 33) the heavy chain variable region of SEQ ID NO: 761 and the light chain variable region of SEQ ID NO: 789; 34) the heavy chain variable region of SEQ ID NO: 762 and the light chain variable region of SEQ ID NO: 790; 35) the heavy chain variable region of SEQ ID NO: 763 and the light chain variable region of SEQ ID NO: 791; 36) the heavy chain variable region of SEQ ID NO: 764 and the light chain variable region of SEQ ID NO: 792; 37) the heavy chain variable region of SEQ ID NO: 765 and the light chain variable region of SEQ ID NO: 793; 38) the heavy chain variable region of SEQ ID NO: 766 and the light chain variable region of SEQ ID NO: 794; 39) the heavy chain variable region of SEQ ID NO: 767 and the light chain variable region of SEQ ID NO: 795; 40) the heavy chain variable region of SEQ ID NO: 768 and the light chain variable region of SEQ ID NO: 796; 41) the heavy chain variable region of SEQ ID NO: 769 and the light chain variable region of SEQ ID NO: 797; 42) the heavy chain variable region of SEQ ID NO: 770 and the light chain variable region of SEQ ID NO: 798; 43) the heavy chain variable region of SEQ ID NO: 771 and the light chain variable region of SEQ ID NO: 799; 44) the heavy chain variable region of SEQ ID NO: 772 and the light chain variable region of SEQ ID NO: 800; 45) the heavy chain variable region of SEQ ID NO: 773 and the light chain variable region of SEQ ID NO: 801; 46) the heavy chain variable region of SEQ ID NO: 774 and the light chain variable region of SEQ ID NO: 802; 47) the heavy chain variable region of SEQ ID NO: 775 and the light chain variable region of SEQ ID NO: 803; 48) the heavy chain variable region of SEQ ID NO: 776 and the light chain variable region of SEQ ID NO: 804; 49) the heavy chain variable region of SEQ ID NO: 777 and the light chain variable region of SEQ ID NO: 805; 50) the heavy chain variable region of SEQ ID NO: 778 and the light chain variable region of SEQ ID NO: 806; 51) the heavy chain variable region of SEQ ID NO: 779 and the light chain variable region of SEQ ID NO: 807; 52) the heavy chain variable region of SEQ ID NO: 780 and the light chain variable region of SEQ ID NO: 808; 53) the heavy chain variable region of SEQ ID NO: 781 and the light chain variable region of SEQ ID NO: 809; 54) the heavy chain variable region of SEQ ID NO: 782 and the light chain variable region of SEQ ID NO: 810; or 55) the heavy chain variable region of SEQ ID NO: 783 and the light chain variable region of SEQ ID NO: 811.

As applied to any of the methods disclosed herein, in some embodiments, the anti-Gal3 antibody or binding fragment thereof comprises the heavy chain (HC) sequence of any one of SEQ ID NOs: 188-200, 202, 205, 468, 470-482. In some embodiments, exemplary HC sequences are depicted in FIG. 26. In some embodiments, any of the methods disclosed herein involving an anti-Gal3 antibody or binding fragment can be performed with an antigen binding molecule that binds to Gal3.

As applied to any of the methods disclosed herein, in some embodiments, the anti-Gal3 antibody or binding fragment thereof comprises the light chain (LC) sequence of any one of SEQ ID NOs: 217-228, 230, 485, 487-499. In some embodiments, exemplary LC sequences are depicted in FIG. 27. In some embodiments, any of the methods disclosed herein involving an anti-Gal3 antibody or binding fragment can be performed with an antigen binding molecule that binds to Gal3.

As applied to any of the methods disclosed herein, in some embodiments, the anti-Gal3 antibody or binding fragment thereof is selected from the group consisting of at least one of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. As applied to any of the methods disclosed herein, in some embodiments, the antibody or binding fragment is selected from the group consisting of: 13H12.2F8, 19D9.2E5, 14H10.2C9, 2D10.2B2, 4A11.2B5, 6H6.2D6, 20H5.A3, 19B5.2E6, 23H9.2E4, 20D11.2C6, 15G7.2A7, 4G2.2G6, 3B11.2G2, 13A12.2E5, 7D8.2D8, 15F10.2D6, 12G5.D7, 24D12.2H9, 13G4.2F8, 9H2.2H10, 23B10.2B12, 6B3.2D3, 846.1F5, 846.2H3, 846T.1H2, IMT-001, 4A11.-H3L1, 4A11.H1L1 and 4A11.H4L2, or binding fragment thereof. In some embodiments, the anti-Gal3 antibody or binding fragment thereof disrupts an interaction between Gal3 and an antibody selected from 2D10.2B2 or 6H6.2D6. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32. In some embodiments, any of the methods disclosed herein involving an anti-Gal3 antibody or binding fragment can be performed with an antigen binding molecule that binds to Gal3.

Also disclosed herein are uses of any one of the anti-Gal3 antibodies or binding fragments disclosed herein in the manufacture of a medicament or composition for the treatment of fibrosis, liver fibrosis, non-alcoholic fatty liver disease (NAFLD), non-alcoholic steatohepatitis (NASH), kidney fibrosis, cardiac fibrosis, arterial fibrosis, venous thrombosis, or pulmonary fibrosis, or an immune-related disorder. In some embodiments, the immune related disorder is sepsis, atopic dermatitis, or psoriasis. In some embodiments, the immune-related disorder is cancer. In some embodiments, the medicament is used as a supplement to PD1/PDL1 blockade therapies or CTLA4 blockade therapies. In some embodiments, the PD1/PDL1 blockade therapies comprise pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and/or BMS-986189. In some embodiments, the CTLA4 blockade therapy comprises ipilimumab and/or tremilimumab.

Also disclosed herein are uses of an anti-Gal3 antibody or binding fragment thereof for the treatment of fibrosis, liver fibrosis, NAFLD, NASH, kidney fibrosis, cardiac fibrosis, arterial fibrosis, venous thrombosis, or pulmonary fibrosis.

Also disclosed herein are uses of an anti-Gal3 antibody or binding fragment thereof for the treatment of cancer. In some embodiments, the cancer is brain cancer, breast cancer, colorectal cancer, kidney cancer, liver cancer, lung cancer, pancreatic cancer, bladder cancer, stomach cancer, or a hematological malignancy.

Also disclosed herein are uses of an anti-Gal3 antibody or binding fragment thereof for the inhibition of tumor cell growth in vitro.

Also disclosed herein are uses of an anti-Gal3 antibody or binding fragment thereof for the retardation of brain tumor growth.

Also disclosed herein are uses of an anti-Gal3 antibody or binding fragment thereof for assisting a payload to cross a blood brain barrier of a subject. In some embodiments, the subject has a neurological disorder.

As applied to any of the uses disclosed herein, the anti-Gal3 antibody or binding fragment thereof is used as a supplement to a standard of care treatment. In some embodiments, the standard of care treatment comprises surgery, radiation, chemotherapy, targeted therapy, immunotherapy, a PD1/PDL1 blockade therapy, a CTLA4 blockade therapy, temozolomide, or any combination thereof.

As applied to any of the uses disclosed herein, in some embodiments, the antibody or binding fragment thereof is formulated for systemic administration. In some embodiments, the antibody or binding fragment thereof is formulated for parenteral administration. In some embodiments, the anti-Gal3 antibody or binding fragment thereof is administered enterally, orally, intranasally, parenterally, intracranially, subcutaneously, intramuscularly, intradermally, or intravenously, or any combination thereof. In some embodiments, the subject is a mammal. In some embodiments, the subject is a human.

As applied to any of the uses disclosed herein, in some embodiments, the anti-Gal3 antibody or binding fragment thereof comprises any one or more sequences (such as a V_{H}-CDR1, V_{H}-CDR2, V_{H}-CDR3, V_{L}-CDR1, V_{L}-CDR2, V_{L}-CDR3, heavy chain variable region, light chain variable region, heavy chain, or light chain sequence) provided throughout this disclosure. In some embodiments, the anti-Gal3 antibody or binding fragment thereof comprises any one or more sequences as shown in FIG. 18-32, including any one or more CDRs, heavy chain variable regions, light chain variable regions, heavy chains, light chains, combinations of CDRs, combinations of variable regions, or combinations of heavy chain and light chain described therein. In some embodiments, the anti-Gal3 antibody or binding fragment thereof comprises a peptide sequence having at least 80%, 85%, 90%, 95%, 99%, or 100% homology to the peptide sequence encoded by any one or more of the nucleic acid sequences as shown in FIG. 37-40, including any nucleic sequences encoding for a heavy chain variable region, light chain variable region, heavy chain, or light chain.

As applied to any of the uses disclosed herein, in some embodiments, the anti-Gal3 antibody or binding fragment thereof binds to one or more peptides of SEQ ID NOs: 3-26. In some embodiments, the anti-Gal3 antibody or binding fragment thereof binds to the N-terminal domain of Gal3, N-terminus of Gal3, or the tandem repeat domain (TRD) of Gal3. In some embodiments, the anti-Gal3 antibody or binding fragment thereof binds to an epitope present within a region of Gal3 defined by Peptide 1 (SEQ ID NO: 3), Peptide 4 (SEQ ID NO: 6), Peptide 6 (SEQ ID NO: 8), Peptide 7 (SEQ ID NO: 9), or any combination thereof. In some embodiments, the antibody or binding fragment thereof binds to an epitope of Gal3 comprising an amino acid sequence of GxYPG, wherein X is alanine, glycine, or valine.

As applied to any of the uses disclosed herein, in some embodiments, the antibody or binding fragment comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3. In some embodiments, the V_{H}-CDR1 comprises an amino acid sequence having at least 60%, at least 70%, at least 80%, at least 90%, or 100% sequence identity to any amino acid sequence according to SEQ ID NOs: 27-36, 397-399, 588-615; the V_{H}-CDR2 comprises an amino acid sequence having at least 60%, at least 70%, at least 80%, at least 90%, or 100% sequence identity to any amino acid sequence according to SEQ ID NOs: 45-54, 400-406, 616-643; the V_{H}-CDR3 comprises an amino acid sequence having at least 60%, at least 70%, at least 80%, at least 90%, or 100% sequence identity to any amino acid sequence according to SEQ ID NOs: 61-69, 71, 408-416, 644-671; the V_{L}-CDR1 comprises an amino acid sequence having at least 60%, at least 70%, at least 80%, at least 90%, or 100% sequence identity to any amino acid sequence according to SEQ ID NOs: 82-92, 417-426, 672-699; the V_{L}-CDR2 comprises an amino acid sequence having at least 60%, at least 70%, at least 80%, at least 90%, or 100% sequence identity to any amino acid sequence according to SEQ ID NOs: 102-111, 427-428, 700-727; and the V_{L}-CDR3 comprises an amino acid sequence having at least 60%, at least 70%, at least 80%, at least 90%, or 100% sequence identity to any amino acid sequence according to SEQ ID NOs: 117-127, 429-434, 728-755.

As applied to any of the uses disclosed herein, in some embodiments, exemplary V_{H}-CDR1 sequences are depicted in FIG. 18. In some embodiments, exemplary V_{H}-CDR2 sequences are depicted in FIG. 19. In some embodiments, exemplary V_{H}-CDR3 sequences are depicted in FIG. 20. In some embodiments, exemplary V_{L}-CDR1 sequences are depicted in FIG. 21. In some embodiments, exemplary V_{L}-CDR2 sequences are depicted in FIG. 22. In some embodiments, exemplary V_{L}-CDR3 sequences are depicted in FIG. 23. In some embodiments, any of the uses disclosed herein involving an anti-Gal3 antibody or binding fragment can be performed with an antigen binding molecule that binds to Gal3.

As applied to any of the uses disclosed herein, in some embodiments, the heavy chain variable region (V_{H}) comprises an amino acid sequence having at least 75%, 80%, 85%, 90%, 95%, or 100% sequence identity to any sequence according to SEQ ID NOs: 136-148, 436, 438-450, 756-783. In some embodiments, the heavy chain variable region is selected from the group consisting of SEQ ID NOs: 136-148, 436, 438-450, 756-783. In some embodiments, exemplary V_{H} are depicted in FIG. 24. In some embodiments, any of the uses disclosed herein involving an anti-Gal3 antibody or binding fragment can be performed with an antigen binding molecule that binds to Gal3.

As applied to any of the uses disclosed herein, in some embodiments, the light chain variable region (V_{L}) comprises an amino acid sequence having at least 75%, 80%, 85%, 90%, 95%, or 100% sequence identity to any sequence according to SEQ ID NOs: 161-172, 174, 451, 453-464, 784-811. In some embodiments, the light chain variable region is selected from the group consisting of SEQ ID NOs: 161-172, 174, 451, 453-464, 784-811. In some embodiments, exemplary V_{L} are depicted in FIG. 25. In some embodiments, any of the uses disclosed herein involving an anti-Gal3 antibody or binding fragment can be performed with an antigen binding molecule that binds to Gal3.

As applied to any of the uses disclosed herein, in some embodiments, the antibody or binding fragment comprises: 1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161; 2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162; 3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163; 4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164; 5) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171; 6) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165; 7) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166; 8) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167; 9) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168; 10) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169; 11) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170; 12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172; 13) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; 14) the V_{H}-CDR1, V_{H}-CDR2, V_{H}-CDR3 of the V_{H}-CDR1, V_{H}-CDR2, V_{H}-CDR3 within SEQ ID NO: 436 and the V_{L}-CDR1, V_{L}-CDR2, V_{L}-CDR3 of the V_{L}-CDR1, V_{L}-CDR2, V_{L}-CDR3 within SEQ ID NO: 451; 15) the V_{H}-CDR1, V_{H}-CDR2, V_{H}-CDR3 of the V_{H}-CDR1, V_{H}-CDR2, V_{H}-CDR3 within SEQ ID NO: 438 and the V_{L}-CDR1, V_{L}-CDR2, V_{L}-CDR3 of the V_{L}-CDR1, V_{L}-CDR2, V_{L}-CDR3 within SEQ ID NO: 453; 16) the V_{H}-CDR1, V_{H}-CDR2, V_{H}-CDR3 of the V_{H}-CDR1, V_{H}-CDR2, V_{H}-CDR3 within SEQ ID NO: 439 and the V_{L}-CDR1, V_{L}-CDR2, V_{L}-CDR3 of the V_{L}-CDR1, V_{L}-CDR2, V_{L}-CDR3 within SEQ ID NO: 162; 17) the V_{H}-CDR1, V_{H}-CDR2, V_{H}-CDR3 of the V_{H}-CDR1, V_{H}-CDR2, V_{H}-CDR3 within SEQ ID NO: 440 and the V_{L}-CDR1, V_{L}-CDR2, V_{L}-CDR3 of the V_{L}-CDR1, V_{L}-CDR2, V_{L}-CDR3 within SEQ ID NO: 454; 18) the V_{H}-CDR1, V_{H}-CDR2, V_{H}-CDR3 of the V_{H}-CDR1, V_{H}-CDR2, V_{H}-CDR3 within SEQ ID NO: 441 and the V_{L}-CDR1, V_{L}-CDR2, V_{L}-CDR3 of the V_{L}-CDR1, V_{L}-CDR2, V_{L}-CDR3 within SEQ ID NO: 455; 19) the V_{H}-CDR1, V_{H}-CDR2, V_{H}-CDR3 of the V_{H}-CDR1, V_{H}-CDR2, V_{H}-CDR3 within SEQ ID NO: 442 and the V_{L}-CDR1, V_{L}-CDR2, V_{L}-CDR3 of the V_{L}-CDR1, V_{L}-CDR2, V_{L}-CDR3 within SEQ ID NO: 456; 20) the V_{H}-CDR1, V_{H}-CDR2, V_{H}-CDR3 of the V_{H}-CDR1, V_{H}-CDR2, V_{H}-CDR3 within SEQ ID NO: 443 and the V_{L}-CDR1, V_{L}-CDR2, V_{L}-CDR3 of the V_{L}-CDR1, V_{L}-CDR2, V_{L}-CDR3 within SEQ ID NO: 457; 21) the V_{H}-CDR1, V_{H}-CDR2, V_{H}-CDR3 of the V_{H}-CDR1, V_{H}-CDR2, V_{H}-CDR3 within SEQ ID NO: 444 and the V_{L}-CDR1, V_{L}-CDR2, V_{L}-CDR3 of the V_{L}-CDR1, V_{L}-CDR2, V_{L}-CDR3 within SEQ ID NO: 458; 22) the V_{H}-CDR1, V_{H}-CDR2, V_{H}-CDR3 of the V_{H}-CDR1, V_{H}-CDR2, V_{H}-CDR3 within SEQ ID NO: 445 and the V_{L}-CDR1, V_{L}-CDR2, V_{L}-CDR3 of the V_{L}-CDR1, V_{L}-CDR2, V_{L}-CDR3 within SEQ ID NO: 459; 23) the V_{H}-CDR1, V_{H}-CDR2, V_{H}-CDR3 of the V_{H}-CDR1, V_{H}-CDR2, V_{H}-CDR3 within SEQ ID NO: 446 and the V_{L}-CDR1, V_{L}-CDR2, V_{L}-CDR3 of the V_{L}-CDR1, V_{L}-CDR2, V_{L}-CDR3 within SEQ ID NO: 460; 24) the V_{H}-CDR1, V_{H}-CDR2, V_{H}-CDR3 of the V_{H}-CDR1, V_{H}-CDR2, V_{H}-CDR3 within SEQ ID NO: 447 and the V_{L}-CDR1, V_{L}-CDR2, V_{L}-CDR3 of the V_{L}-CDR1, V_{L}-CDR2, V_{L}-CDR3 within SEQ ID NO: 461; 25) the V_{H}-CDR1, V_{H}-CDR2, V_{H}-CDR3 of the V_{H}-CDR1, V_{H}-CDR2, V_{H}-CDR3 within SEQ ID NO: 448 and the V_{L}-CDR1, V_{L}-CDR2, V_{L}-CDR3 of the V_{L}-CDR1, V_{L}-CDR2, V_{L}-CDR3 within SEQ ID NO: 462; 26) the V_{H}-CDR1, V_{H}-CDR2, V_{H}-CDR3 of the V_{H}-CDR1, V_{H}-CDR2, V_{H}-CDR3 within SEQ ID NO: 449 and the V_{L}-CDR1, V_{L}-CDR2, V_{L}-CDR3 of the V_{L}-CDR1, V_{L}-CDR2, V_{L}-CDR3 within SEQ ID NO: 463; 27) the V_{H}-CDR1, V_{H}-CDR2, V_{H}-CDR3 of the V_{H}-CDR1, V_{H}-CDR2, V_{H}-CDR3 within SEQ ID NO: 450 and the V_{L}-CDR1, V_{L}-CDR2, V_{L}-CDR3 of the V_{L}-CDR1, V_{L}-CDR2, V_{L}-CDR3 within SEQ ID NO: 464; 28) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784; 29) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785; 30) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786; 31) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787; 32) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788; 33) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789; 34) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790; 35) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791; 36) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792; 37) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793; 38) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794; 39) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795; 40) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796; 41) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797; 42) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798; 43) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799; 44) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800; 45) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801; 46) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802; 47) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803; 48) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804; 49) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805; 50) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806; 51) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807; 52) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808; 53) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809; 54) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 55) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

As applied to any of the uses disclosed herein, in some embodiments, the antibody or binding fragment comprises: 1) the heavy chain variable region of SEQ ID NO: 136 and the light chain variable region of SEQ ID NO: 161; 2) the heavy chain variable region of SEQ ID NO: 137 and the light chain variable region of SEQ ID NO: 162; 3) the heavy chain variable region of SEQ ID NO: 138 and the light chain variable region of SEQ ID NO: 163; 4) the heavy chain variable region of SEQ ID NO: 139 and the light chain variable region of SEQ ID NO: 164; 5) the heavy chain variable region of SEQ ID NO: 139 and the light chain variable region of SEQ ID NO: 171; 6) the heavy chain variable region of SEQ ID NO: 140 and the light chain variable region of SEQ ID NO: 165; 7) the heavy chain variable region of SEQ ID NO: 141 and the light chain variable region of SEQ ID NO: 166; 8) the heavy chain variable region of SEQ ID NO: 142 and the light chain variable region of SEQ ID NO: 167; 9) the heavy chain variable region of SEQ ID NO: 143 and the light chain variable region of SEQ ID NO: 168; 10) the heavy chain variable region of SEQ ID NO: 144 and the light chain variable region of SEQ ID NO: 169; 11) the heavy chain variable region of SEQ ID NO: 145 and the light chain variable region of SEQ ID NO: 170; 12) the heavy chain variable region of SEQ ID NO: 146 and the light chain variable region of SEQ ID NO: 172; 13) the heavy chain variable region of SEQ ID NO: 148 and the light chain variable region of SEQ ID NO: 174; 14) the heavy chain variable region of SEQ ID NO: 436 and the light chain variable region of SEQ ID NO: 451; 15) the heavy chain variable region of SEQ ID NO: 438 and the light chain variable region of SEQ ID NO: 453; 16) the heavy chain variable region of SEQ ID NO: 439 and the light chain variable region of SEQ ID NO: 162; 17) the heavy chain variable region of SEQ ID NO: 440 and the light chain variable region of SEQ ID NO: 454; 18) the heavy chain variable region of SEQ ID NO: 441 and the light chain variable region of SEQ ID NO: 455; 19) the heavy chain variable region of SEQ ID NO: 442 and the light chain variable region of SEQ ID NO: 456; 20) the heavy chain variable region of SEQ ID NO: 443 and the light chain variable region of SEQ ID NO: 457; 21) the heavy chain variable region of SEQ ID NO: 444 and the light chain variable region of SEQ ID NO: 458; 22) the heavy chain variable region of SEQ ID NO: 445 and the light chain variable region of SEQ ID NO: 459; 23) the heavy chain variable region of SEQ ID NO: 446 and the light chain variable region of SEQ ID NO: 460; 24) the heavy chain variable region of SEQ ID NO: 447 and the light chain variable region of SEQ ID NO: 461; 25) the heavy chain variable region of SEQ ID NO: 448 and the light chain variable region of SEQ ID NO: 462; 26) the heavy chain variable region of SEQ ID NO: 449 and the light chain variable region of SEQ ID NO: 463; or 27) the heavy chain variable region of SEQ ID NO: 450 and the light chain variable region of SEQ ID NO: 464; 28) the heavy chain variable region of SEQ ID NO: 756 and the light chain variable region of SEQ ID NO: 784; 29) the heavy chain variable region of SEQ ID NO: 757 and the light chain variable region of SEQ ID NO: 785; 30) the heavy chain variable region of SEQ ID NO: 758 and the light chain variable region of SEQ ID NO: 786; 31) the heavy chain variable region of SEQ ID NO: 759 and the light chain variable region of SEQ ID NO: 787; 32) the heavy chain variable region of SEQ ID NO: 760 and the light chain variable region of SEQ ID NO: 788; 33) the heavy chain variable region of SEQ ID NO: 761 and the light chain variable region of SEQ ID NO: 789; 34) the heavy chain variable region of SEQ ID NO: 762 and the light chain variable region of SEQ ID NO: 790; 35) the heavy chain variable region of SEQ ID NO: 763 and the light chain variable region of SEQ ID NO: 791; 36) the heavy chain variable region of SEQ ID NO: 764 and the light chain variable region of SEQ ID NO: 792; 37) the heavy chain variable region of SEQ ID NO: 765 and the light chain variable region of SEQ ID NO: 793; 38) the heavy chain variable region of SEQ ID NO: 766 and the light chain variable region of SEQ ID NO: 794; 39) the heavy chain variable region of SEQ ID NO: 767 and the light chain variable region of SEQ ID NO: 795; 40) the heavy chain variable region of SEQ ID NO: 768 and the light chain variable region of SEQ ID NO: 796; 41) the heavy chain variable region of SEQ ID NO: 769 and the light chain variable region of SEQ ID NO: 797; 42) the heavy chain variable region of SEQ ID NO: 770 and the light chain variable region of SEQ ID NO: 798; 43) the heavy chain variable region of SEQ ID NO: 771 and the light chain variable region of SEQ ID NO: 799; 44) the heavy chain variable region of SEQ ID NO: 772 and the light chain variable region of SEQ ID NO: 800; 45) the heavy chain variable region of SEQ ID NO: 773 and the light chain variable region of SEQ ID NO: 801; 46) the heavy chain variable region of SEQ ID NO: 774 and the light chain variable region of SEQ ID NO: 802; 47) the heavy chain variable region of SEQ ID NO: 775 and the light chain variable region of SEQ ID NO: 803; 48) the heavy chain variable region of SEQ ID NO: 776 and the light chain variable region of SEQ ID NO: 804; 49) the heavy chain variable region of SEQ ID NO: 777 and the light chain variable region of SEQ ID NO: 805; 50) the heavy chain variable region of SEQ ID NO: 778 and the light chain variable region of SEQ ID NO: 806; 51) the heavy chain variable region of SEQ ID NO: 779 and the light chain variable region of SEQ ID NO: 807; 52) the heavy chain variable region of SEQ ID NO: 780 and the light chain variable region of SEQ ID NO: 808; 53) the heavy chain variable region of SEQ ID NO: 781 and the light chain variable region of SEQ ID NO: 809; 54) the heavy chain variable region of SEQ ID NO: 782 and the light chain variable region of SEQ ID NO: 810; or 55) the heavy chain variable region of SEQ ID NO: 783 and the light chain variable region of SEQ ID NO: 811.

As applied to any of the uses disclosed herein, in some embodiments, the anti-Gal3 antibody or binding fragment thereof comprises the heavy chain (HC) sequence of any one of SEQ ID NOs: 188-200, 202, 205, 468, 470-482. In some embodiments, exemplary HC sequences are depicted in FIG. 26. In some embodiments, any of the uses disclosed herein involving an anti-Gal3 antibody or binding fragment can be performed with an antigen binding molecule that binds to Gal3.

As applied to any of the uses disclosed herein, in some embodiments, the anti-Gal3 antibody or binding fragment thereof comprises the light chain (LC) sequence of any one of SEQ ID NOs: 217-228, 230, 485, 487-499. In some embodiments, exemplary LC sequences are depicted in FIG. 27. In some embodiments, any of the uses disclosed herein involving an anti-Gal3 antibody or binding fragment can be performed with an antigen binding molecule that binds to Gal3.

As applied to any of the uses disclosed herein, in some embodiments, the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. As applied to any of the uses disclosed herein, in some embodiments, the antibody or binding fragment is selected from the group consisting of: 13H12.2F8, 19D9.2E5, 14H10.2C9, 2D10.2B2, 4A11.2B5, 6H6.2D6, 20H5.A3, 19B5.2E6, 23H9.2E4, 20D11.2C6, 15G7.2A7, 4G2.2G6, 3B11.2G2, 13A12.2E5, 7D8.2D8, 15F10.2D6, 12G5.D7, 24D12.2H9, 13G4.2F8, 9H2.2H10, 23B10.2B12, 6B3.2D3, 846.1F5, 846.2H3, 846T.1H2, TB001 (IMT001), TB006 (4A11.H3L1), 4A11.H1L1 and 4A11.H4L2, or binding fragment thereof. In some embodiments, the anti-Gal3 antibody or binding fragment thereof disrupts an interaction between Gal3 and an antibody selected from 2D10.2B2 or 6H6.2D6. In some embodiments, the heavy and light chain CDRs associated with each of the foregoing antibodies are depicted in FIG. 30. In some embodiments, the V_{H} and V_{L} associated with each of the foregoing antibodies are depicted in FIG. 31. In some embodiments, the HC and LC associated with each of the foregoing antibodies are depicted in FIG. 32. In some embodiments, any of the uses disclosed herein involving an anti-Gal3 antibody or binding fragment can be performed with an antigen binding molecule that binds to Gal3.

### Antibody Production

In some cases, anti-Gal3 antibodies or binding fragments thereof are raised by standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.). When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Polyclonal or monoclonal anti-Gal3 antibodies or binding fragments thereof can be produced from animals which have been genetically altered to produce human immunoglobulins. A transgenic animal can be produced by initially producing a “knock-out” animal which does not produce the animal's natural antibodies, and stably transforming the animal with a human antibody locus (e.g., by the use of a human artificial chromosome). In such cases, only human antibodies are then made by the animal. Techniques for generating such animals, and deriving antibodies therefrom, are described in U.S. Pat. Nos. 6,162,963 and 6,150,584, each incorporated fully herein by reference in its entirety. Such antibodies can be referred to as human xenogeneic antibodies.

Alternatively, anti-Gal3 antibodies or binding fragments thereof can be produced from phage libraries containing human variable regions. See U.S. Pat. No. 6,174,708, incorporated fully herein by reference in its entirety.

In some aspects of any of the embodiments disclosed herein, an anti-Gal3 antibody or binding fragment thereof is produced by a hybridoma.

For monoclonal anti-Gal3 antibodies, hybridomas may be formed by isolating the stimulated immune cells, such as those from the spleen of the inoculated animal. These cells can then be fused to immortalized cells, such as myeloma cells or transformed cells, which are capable of replicating indefinitely in cell culture, thereby producing an immortal, immunoglobulin-secreting cell line. The immortal cell line utilized can be selected to be deficient in enzymes necessary for the utilization of certain nutrients. Many such cell lines (such as myelomas) are known to those skilled in the art, and include, for example: thymidine kinase (TK) or hypoxanthine-guanine phosphoriboxyl transferase (HGPRT). These deficiencies allow selection for fused cells according to their ability to grow on, for example, hypoxanthine aminopterinthymidine medium (HAT).

In addition, the anti-Gal3 antibody or binding fragment thereof may be produced by genetic engineering.

Anti-Gal3 antibodies or binding fragments thereof disclosed herein can have a reduced propensity to induce an undesired immune response in humans, for example, anaphylactic shock, and can also exhibit a reduced propensity for priming an immune response which would prevent repeated dosage with an antibody therapeutic or imaging agent (e.g., the human-anti-murine-antibody “HAMA” response). Such anti-Gal3 antibodies or binding fragments thereof include, but are not limited to, humanized, chimeric, or xenogeneic human anti-Gal3 antibodies or binding fragments thereof.

Chimeric anti-Gal3 antibodies or binding fragments thereof can be made, for example, by recombinant means by combining the murine variable light and heavy chain regions (VK and VH), obtained from a murine (or other animal-derived) hybridoma clone, with the human constant light and heavy chain regions, in order to produce an antibody with predominantly human domains. The production of such chimeric antibodies is well known in the art and may be achieved by standard means (as described, e.g., in U.S. Pat. No. 5,624,659, incorporated fully herein by reference).

The term “humanized” as applies to a non-human (e.g. rodent or primate) antibodies are hybrid immunoglobulins, immunoglobulin chains or fragments thereof which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, rabbit or primate having the desired specificity, affinity and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance and minimize immunogenicity when introduced into a human body. In some examples, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody may also comprise at least a portion of an immunoglobulin constant region (Fc), typically that of a human immunoglobulin.

Humanized antibodies can be engineered to contain human-like immunoglobulin domains and incorporate only the complementarity-determining regions of the animal-derived antibody. This can be accomplished by carefully examining the sequence of the hyper-variable loops of the variable regions of a monoclonal antigen binding unit or monoclonal antibody and fitting them to the structure of a human antigen binding unit or human antibody chains. See, e.g., U.S. Pat. No. 6,187,287, incorporated fully herein by reference.

Methods for humanizing non-human antibodies are well known in the art. “Humanized” antibodies are antibodies in which at least part of the sequence has been altered from its initial form to render it more like human immunoglobulins. In some versions, the heavy (H) chain and light (L) chain constant (C) regions are replaced with human sequence. This can be a fusion polypeptide comprising a variable (V) region and a heterologous immunoglobulin C region. In some versions, the complementarity determining regions (CDRs) comprise non-human antibody sequences, while the V framework regions have also been converted to human sequences. See, for example, EP 0329400. In some versions, V regions are humanized by designing consensus sequences of human and mouse V regions and converting residues outside the CDRs that are different between the consensus sequences.

In principle, a framework sequence from a humanized antibody can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a human antibody (HuAb) is to the original murine antibody (muAb), the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The disclosure thus encompasses HuAbs with different variable (V) regions. It is within the skill of one in the art to determine suitable V region sequences and to optimize these sequences. Methods for obtaining antibodies with reduced immunogenicity are also described in U.S. Pat. No. 5,270,202 and EP 699,755, each hereby incorporated by reference in its entirety.

Humanized antibodies can be prepared by a process of analysis of the parental sequences and various conceptual humanized products using three dimensional models of the parental and humanized sequences. Three-dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved.

A process for humanization of subject antigen binding units can be as follows. The best-fit germline acceptor heavy and light chain variable regions are selected based on homology, canonical structure and physical properties of the human antibody germlines for grafting. Computer modeling of mVH/VL versus grafted hVH/VL is performed and prototype humanized antibody sequence is generated. If modeling indicated a need for framework back-mutations, second variant with indicated FW changes is generated. DNA fragments encoding the selected germline frameworks and murine CDRs are synthesized. The synthesized DNA fragments are subcloned into IgG expression vectors and sequences are confirmed by DNA sequencing. The humanized antibodies are expressed in cells, such as 293F and the proteins are tested, for example in MDM phagocytosis assays and antigen binding assays. The humanized antigen binding units are compared with parental antigen binding units in antigen binding affinity, for example, by FACS on cells expressing the target antigen. If the affinity is greater than 2-fold lower than parental antigen binding unit, a second round of humanized variants can be generated and tested as described above.

In some instances, the anti-Gal3 antibody or binding fragment thereof is a bispecific antibody or binding fragment thereof. Exemplary bispecific antibody formats include, but are not limited to, Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), Azymetric, Biclonics, Fab-scFv-Fc, Two-in-one/Dual Action Fab (DAF), FinomAb, scFv-Fc-(Fab)-fusion, Dock-aNd-Lock (DNL), Tandem diAbody (TandAb), Dual-affinity-ReTargeting (DART), nanobody, triplebody, tandems scFv (taFv), triple heads, tandem dAb/VHH, triple dAb/VHH, or tetravalent dAb/VHH. In some cases, the anti-Gal3 antibody or binding fragment thereof is a bispecific antibody or binding fragment thereof comprising a bispecific antibody format illustrated in Brinkmann and Kontermann, “The making of bispecific antibodies,” MABS 9(2): 182-212 (2017).

In some embodiments, the anti-Gal3 antibody or binding fragment thereof can comprise an IgM, IgG (e.g., IgG1, IgG2, IgG3, or IgG4), IgA, or IgE framework. The IgG framework can be IgG1, IgG2, IgG3 or IgG4. In some cases, the anti-Gal3 antibody or binding fragment thereof comprises an IgG1 framework. In some cases, the anti-Gal3 antibody or binding fragment thereof comprises an IgG2 framework. In some cases, the anti-Gal3 antibody or binding fragment thereof comprises an IgG4 framework. The anti-Gal3 antibody or binding fragment thereof can further comprise a Fc mutation.

In some embodiments, the Fc region comprises one or more mutations that modulate Fc receptor interactions, e.g., to enhance effector functions such as ADCC and/or CDC. In such instances, exemplary residues when mutated modulate effector functions include S239, K326, A330, 1332, or E333, in which the residue position correspond to IgG1 and the residue numbering is in accordance to Kabat numbering (EU index of Kabat et al 1991 Sequences of Proteins of Immunological Interest). In some instances, the one or more mutations comprise S239D, K326W, A330L, 1332E, E333A, E333S, or a combination thereof. In some cases, the one or more mutations comprise S239D, 1332E, or a combination thereof. In some cases, the one or more mutations comprise S239D, A330L, 1332E, or a combination thereof. In some cases, the one or more mutations comprise K326W, E333S, or a combination thereof. In some cases, the mutation comprises E333A.

In some embodiments, an anti-Gal3 antibody or binding fragment thereof can be either “monospecific” or “multi-specific”. Multi-specific anti-Gal3 antibodies or binding fragments thereof can be further classified on the basis of their binding specificities. A “monospecific” anti-Gal3 antibody or binding fragment thereof is a molecule capable of binding to one or more antigens of the same kind. A “multi-specific” anti-Gal3 antibody or binding fragment thereof is a molecule having binding specificities for at least two different antigens. While such molecules normally will only bind two distinct antigens (i.e. bispecific anti-Gal3 antibodies), antibodies with additional specificities (e.g. tri-specific, tetra-specific, and so on) are encompassed by this expression when used herein. This disclosure further provides multi-specific anti-Gal3 antibodies. Multi-specific anti-Gal3 antibodies or binding fragments thereof are multi-specific molecules capable of binding to at least two distinct antigens, e.g., bispecific and tri-specific molecules exhibiting binding specificities to two and three distinct antigens, respectively, where at least one antigen is not Gal3 or any portion, fragment, derivative, or modification thereof.

In some embodiments are methods directed to screening for or identifying antibodies capable of disrupting an interaction between Gal3 and a protein. In some embodiments, the protein is any of the proteins that interact with Gal3 disclosed herein or any protein associated with any of the diseases, disorders, or conditions disclosed herein. In some embodiments, the methods may comprise: (a) contacting Gal3 protein with an antibody or binding fragment thereof that selectively binds to Gal3, thereby forming a Gal3-antibody complex; (b) contacting the Gal3-antibody complex with the protein; (c) removing unbound protein; and (d) detecting protein bound to the Gal3-antibody complex, wherein the antibody is capable of disrupting an interaction of Gal3 and the protein when the protein is not detected or detected in relatively low amounts in (d). In some embodiments, the method comprises an immunoassay. In some embodiments, the immunoassay is an enzyme-linked immunosorbent assay (ELISA).

In some embodiments of the screening methods, the protein that interacts with Gal3 is a protein associated with a neurological disorder or proteopathy, such as but not limited to APP695, Aβ42, TLR4, TREM2, Tau, α-synuclein, or any combination thereof. In some embodiments, the methods comprise (a) contacting Gal3 protein with an antibody or binding fragment thereof that selectively binds to Gal3, thereby forming a Gal3-antibody complex; (b) contacting the Gal3-antibody complex with APP695, Aβ42, TLR4, TREM2, Tau, α-synuclein, or any combination thereof; (c) removing unbound APP695, Aβ42, TLR4, TREM2, Tau, α-synuclein, or any combination thereof; and (d) detecting APP695, Aβ42, TLR4, TREM2, Tau, α-synuclein, or any combination thereof, bound to the Gal3-antibody complex, wherein the antibody is capable of disrupting an interaction of Gal3 and the APP695, Aβ42, TLR4, TREM2, Tau, α-synuclein, or any combination thereof, when the APP695, Aβ42, TLR4, TREM2, Tau, α-synuclein, or any combination thereof, is not detected or detected in relatively low amounts in (d). In some embodiments, the method comprises an immunoassay. In some embodiments, the immunoassay is an enzyme-linked immunosorbent assay (ELISA).

In some embodiments of the screening methods, the protein that interacts with Gal3 is a cell surface marker or associated with an inflammatory disease, cancer, or fibrosis, such as but not limited to a TGF-b receptor, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof. In some embodiments, the methods comprise (a) contacting Gal3 protein with an antibody or binding fragment thereof that selectively binds to Gal3, thereby forming a Gal3-antibody complex; (b) contacting the Gal3-antibody complex with TGF-b receptor protein, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof; (c) removing unbound TGF-b receptor protein, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof; and (d) detecting TGF-b receptor protein, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof, bound to the Gal3-antibody complex, wherein the antibody is capable of disrupting an interaction of Gal3 and the TGF-b receptor protein, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof, when the TGF-b receptor protein, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof, is not detected or detected in relatively low amounts in (d). In some embodiments, the method comprises an immunoassay. In some embodiments, the immunoassay is an enzyme-linked immunosorbent assay (ELISA).

In some embodiments, the methods for screening for or identifying antibodies capable of disrupting an interaction between Gal3 and a protein is a cell-based assay. In some embodiments, the cell-based assay methods comprise using a reporter cell line. In some embodiments, the cell-based assay methods comprise providing a reporter cell line wherein a promoter region of a representative downstream target gene of a protein is cloned upstream of a reporter construct (e.g. luciferase, fluorescent protein, GFP, alkaline phosphatase) and contacting the reporter cell line with an anti-Gal3 antibody. In some embodiments, addition of the anti-Gal3 antibody affects expression (e.g. increase or decrease) of this reporter construct if Gal3 is involved in a pathway regulated by the protein. In some embodiments, the effect on expression is measured by the reporter construct. Antibodies that have the strongest blocking activity are selected. In some embodiments, the protein is APP695, Aβ42, TLR4, TREM2, Tau, α-synuclein, a TGF-b receptor protein, VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, or FGFR4, or any combination thereof, and the representative downstream target gene is a gene that is regulated by the protein.

### Polynucleotides and Vectors

In some embodiments, the present disclosure provides isolated nucleic acids encoding any of the anti-Gal3 antibodies or binding fragments thereof disclosed herein. In another embodiment, the present disclosure provides vectors comprising a nucleic acid sequence encoding any anti-Gal3 antibody or binding fragment thereof disclosed herein. In some embodiments, this disclosure provides isolated nucleic acids that encode a light-chain CDR and a heavy-chain CDR of an anti-Gal3 antibody or binding fragment thereof disclosed herein.

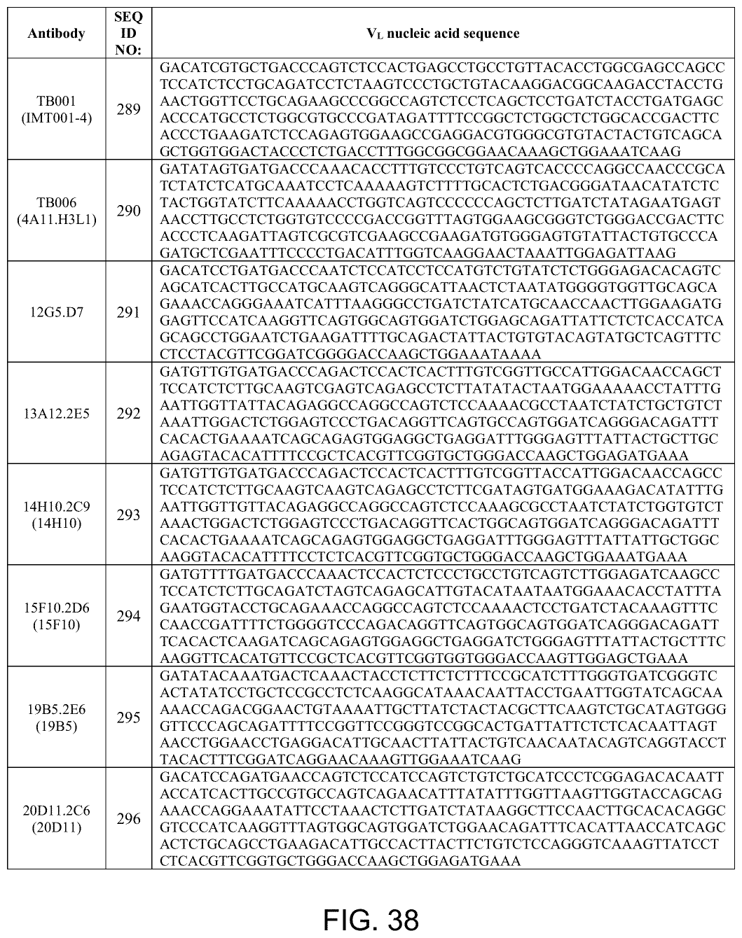

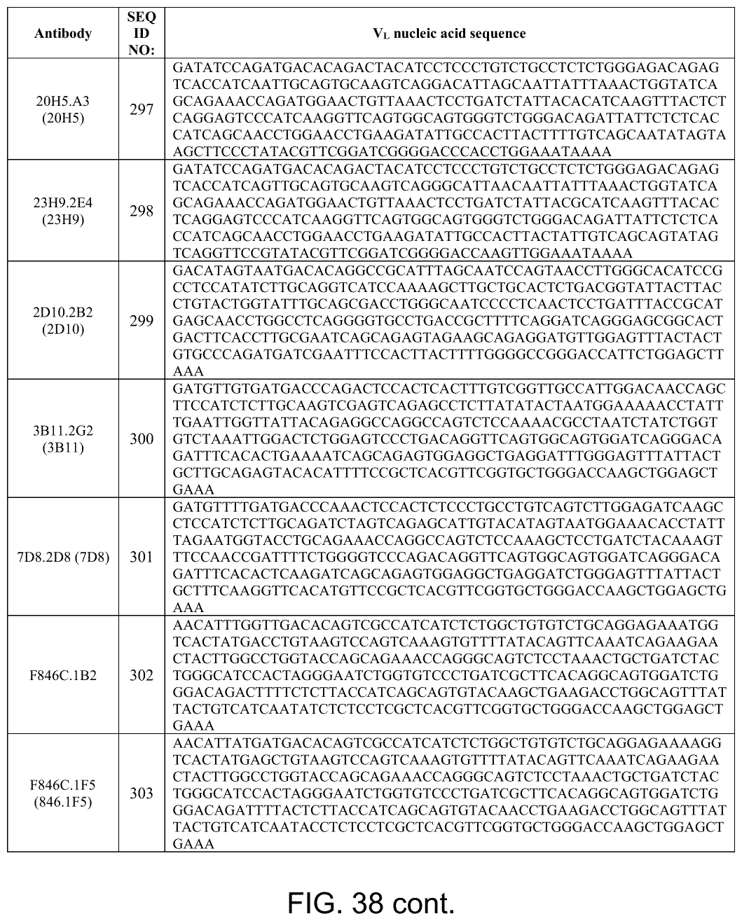

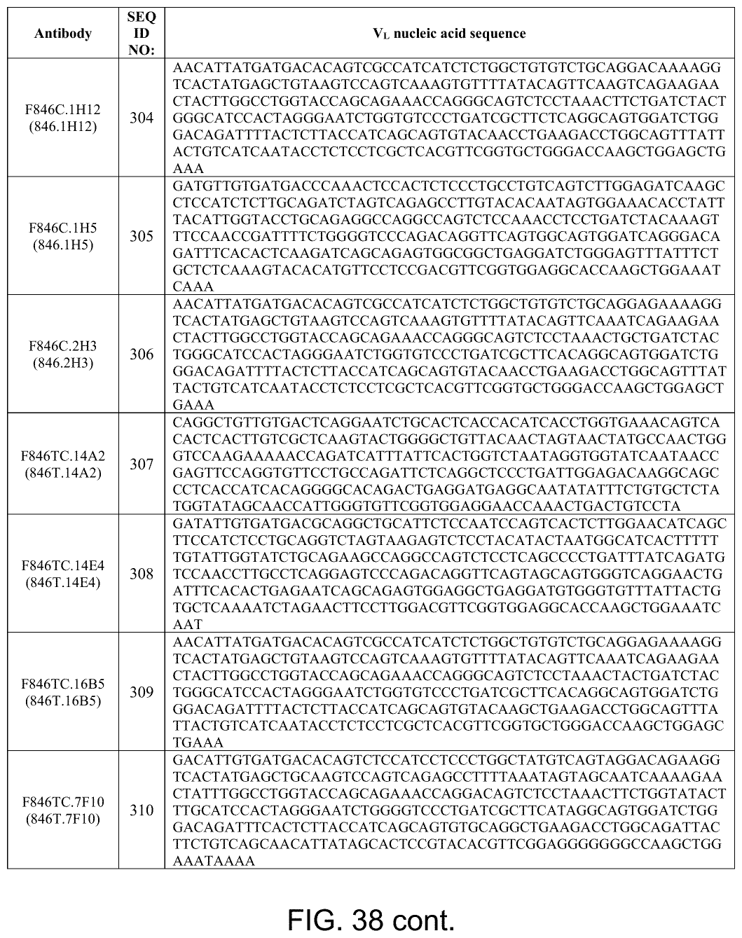

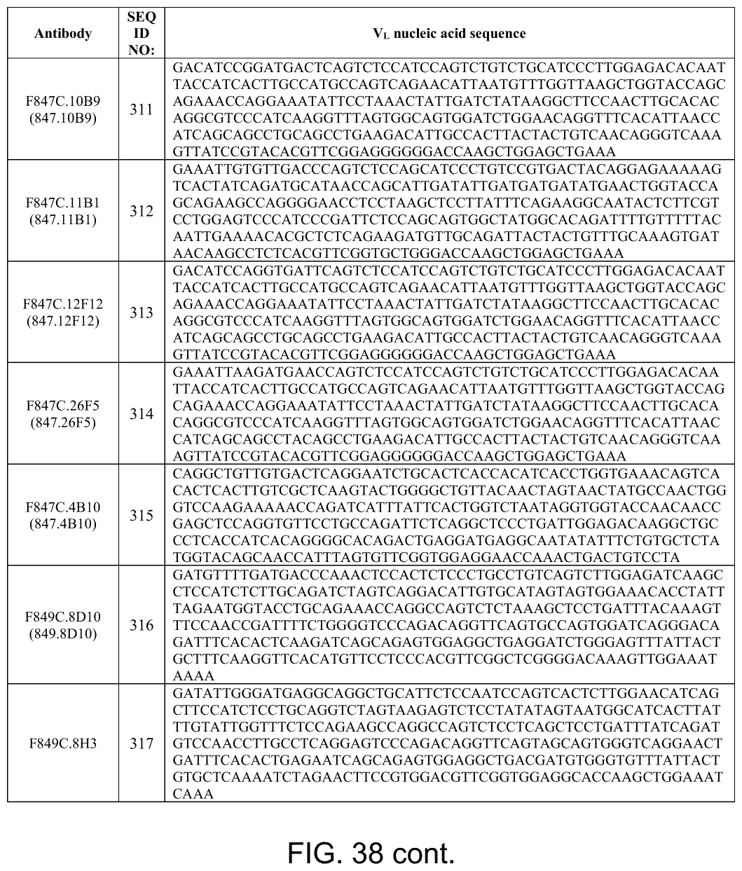

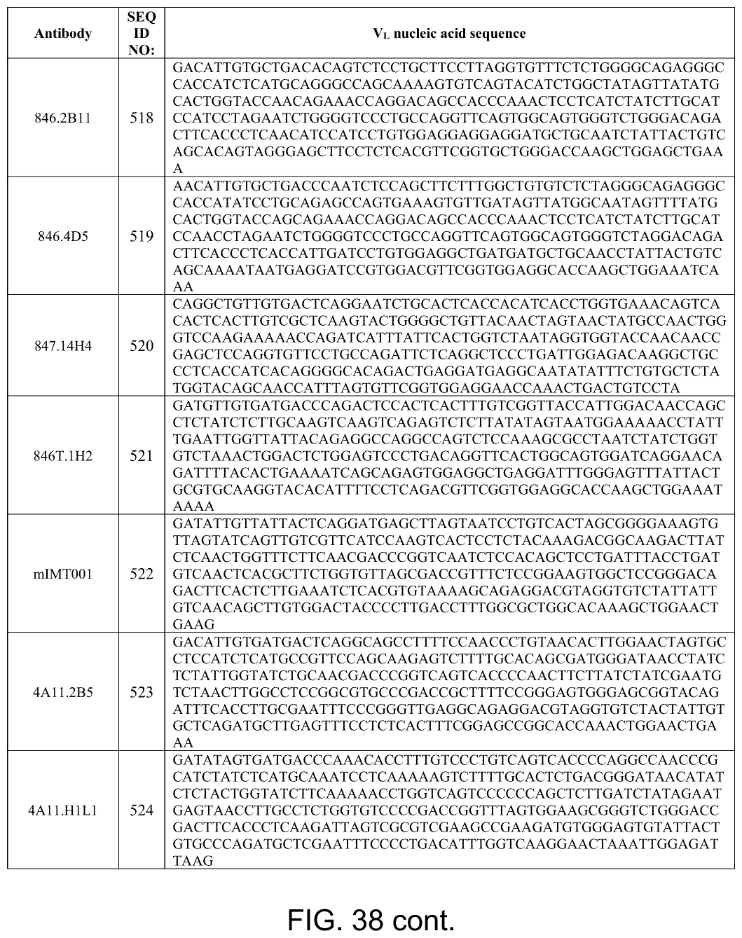

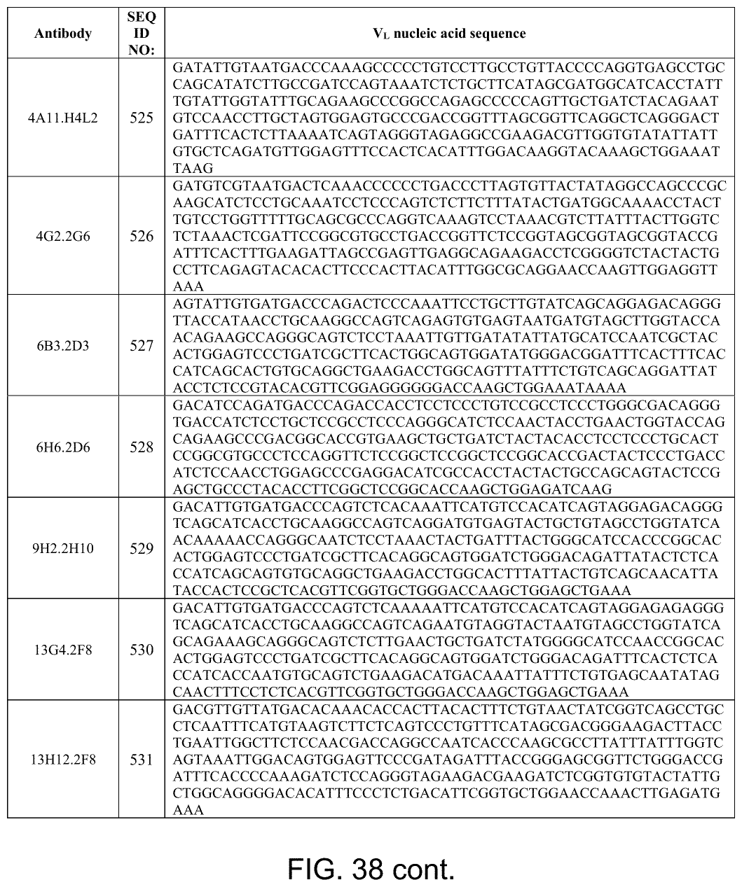

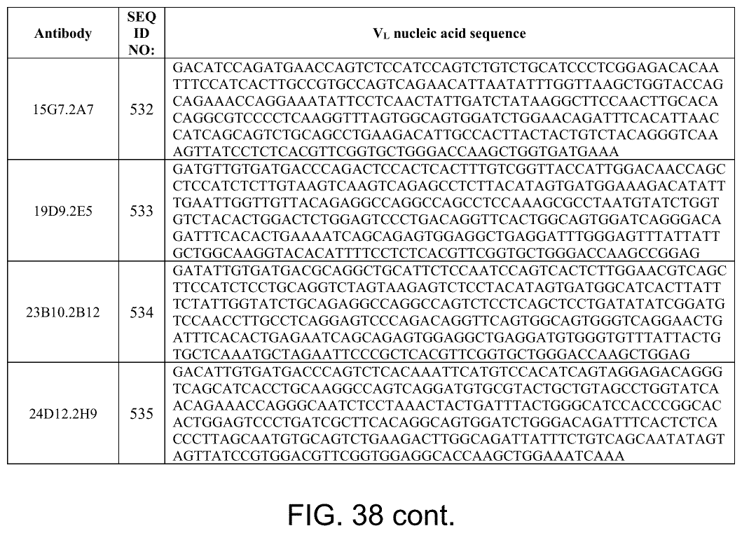

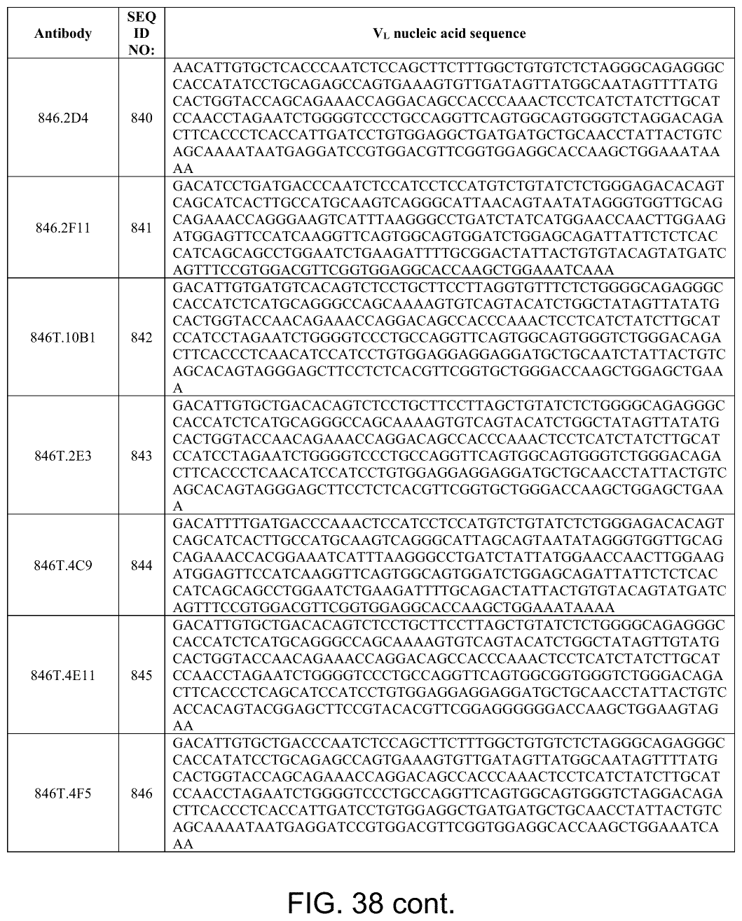

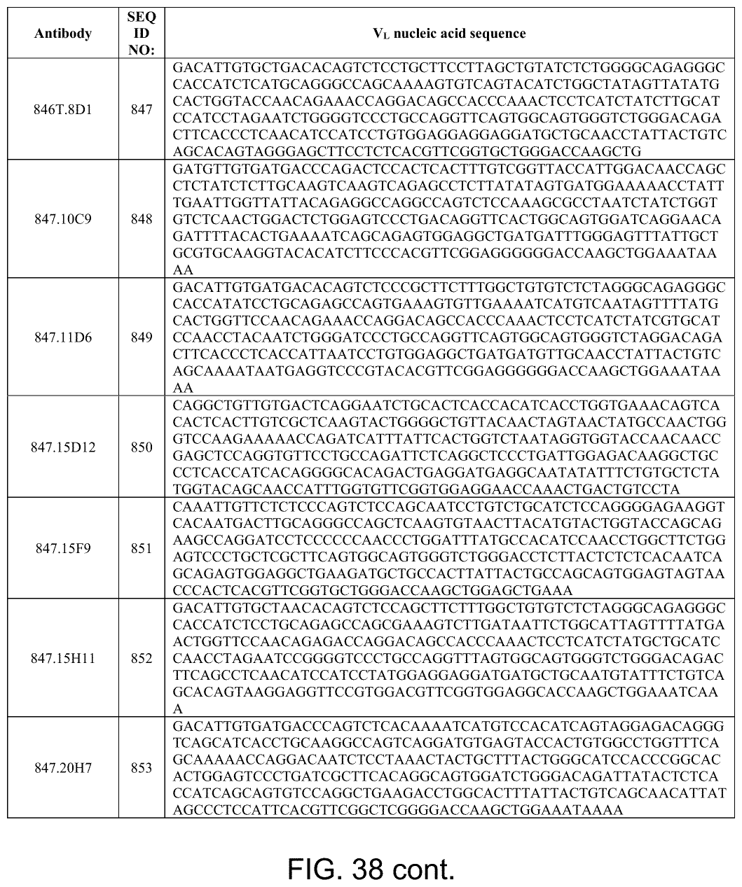

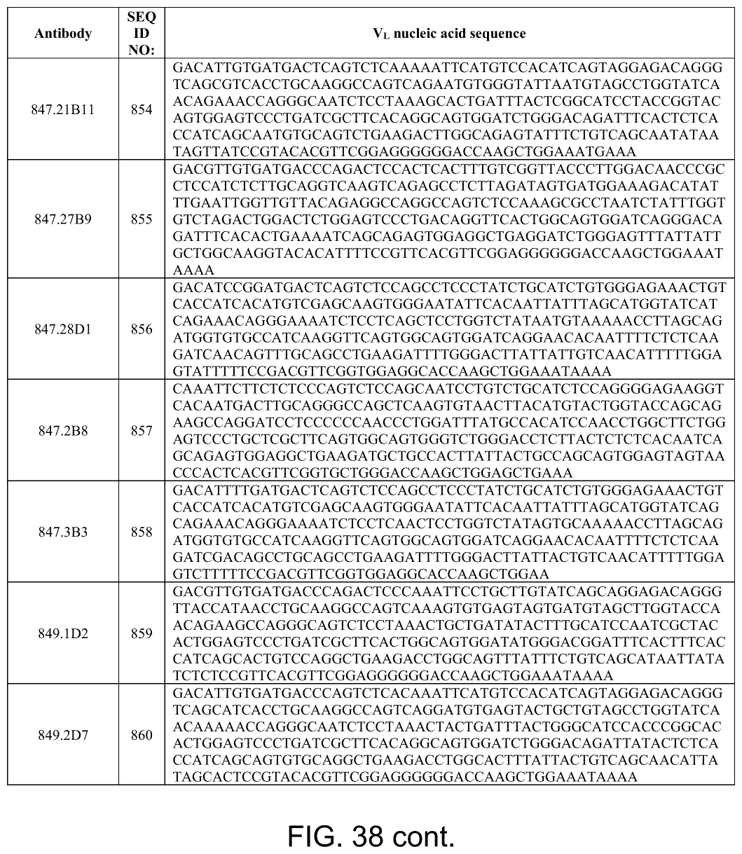

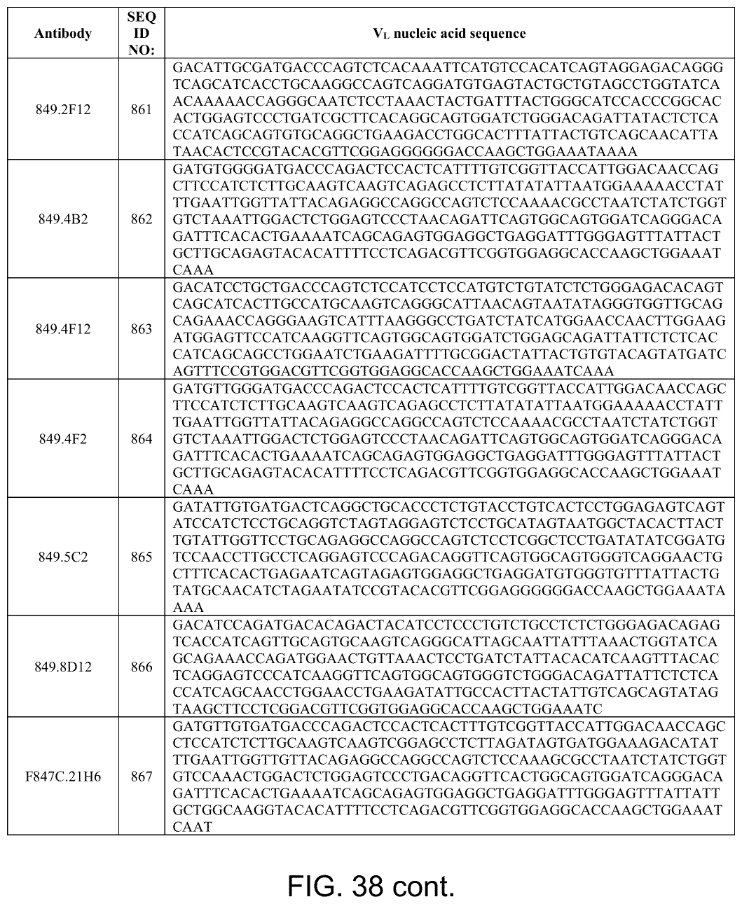

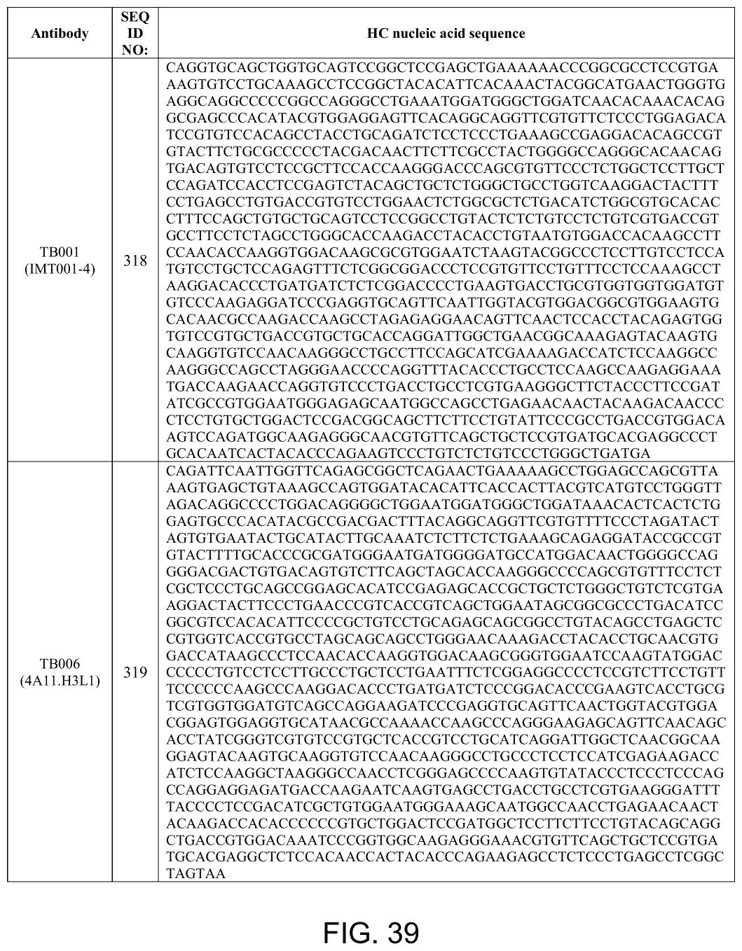

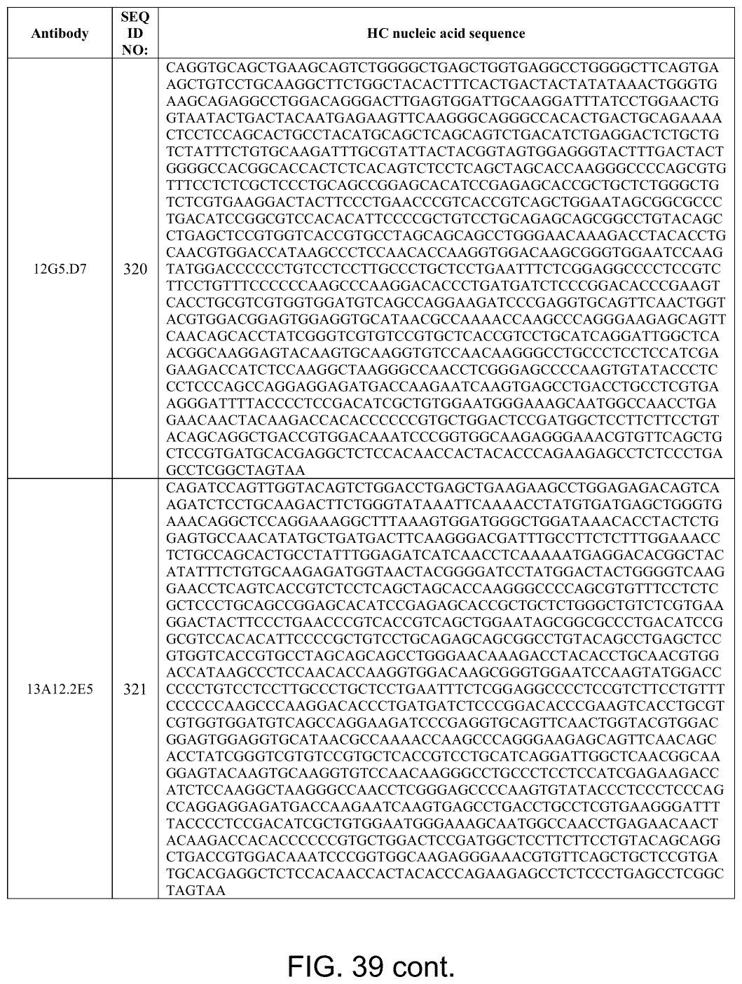

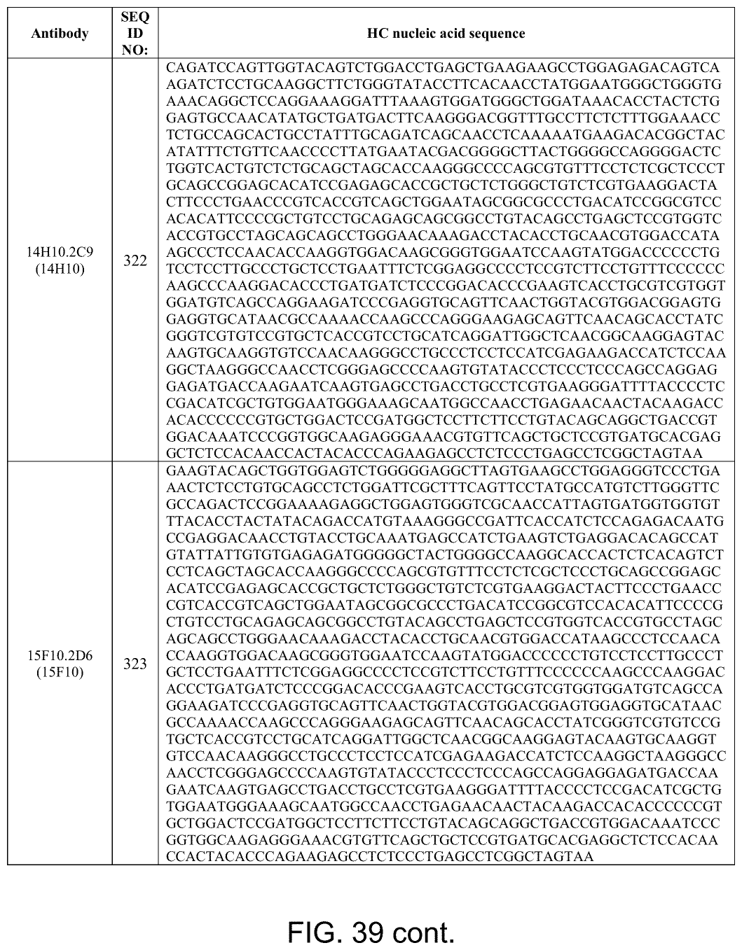

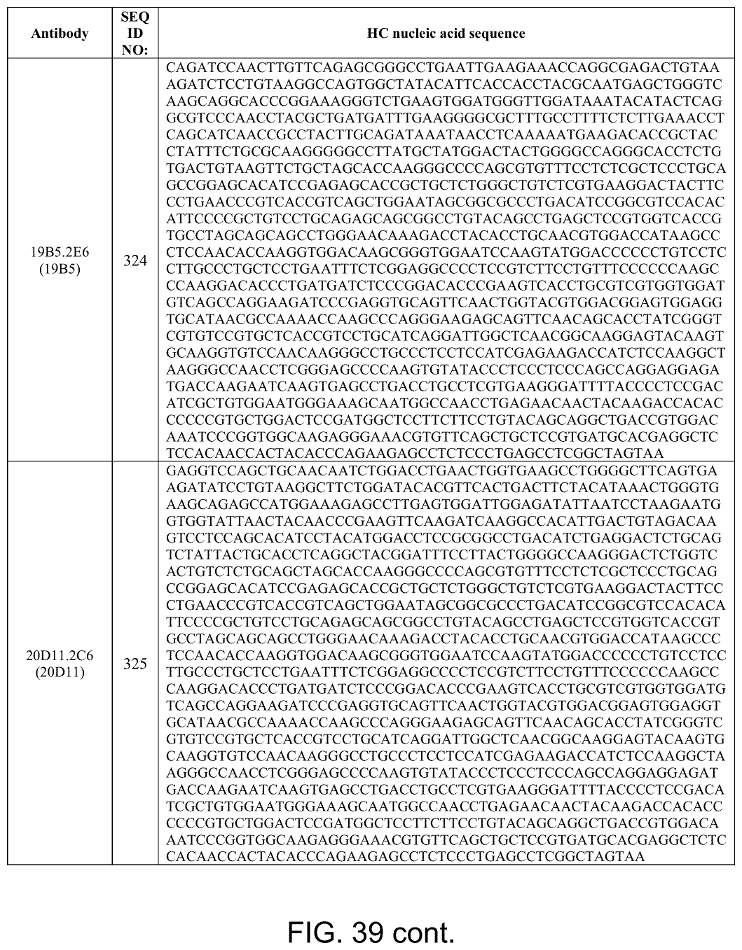

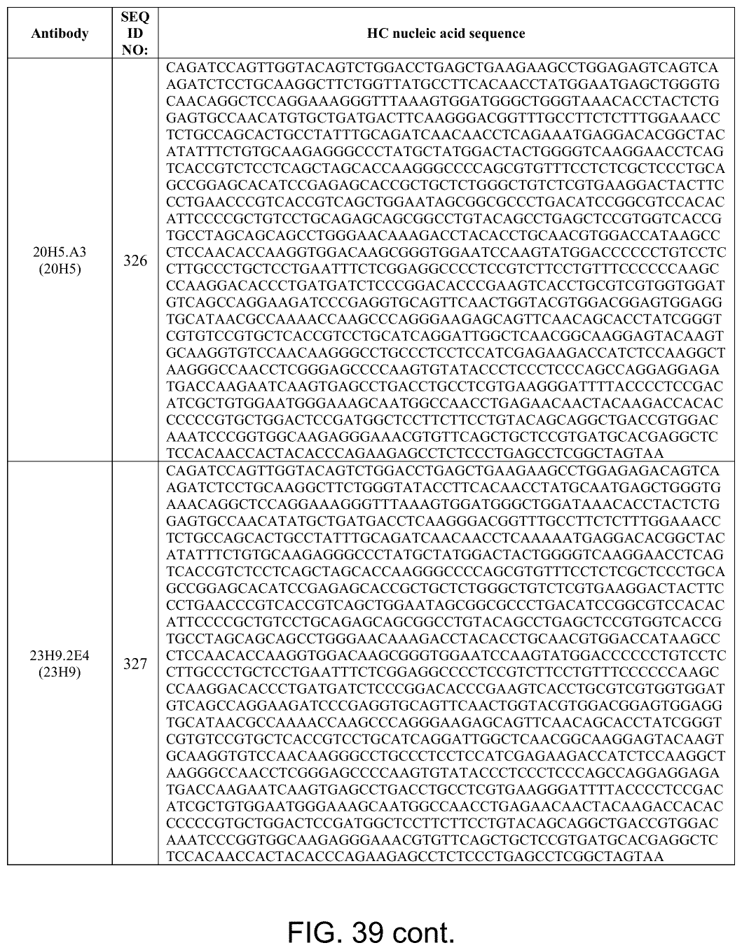

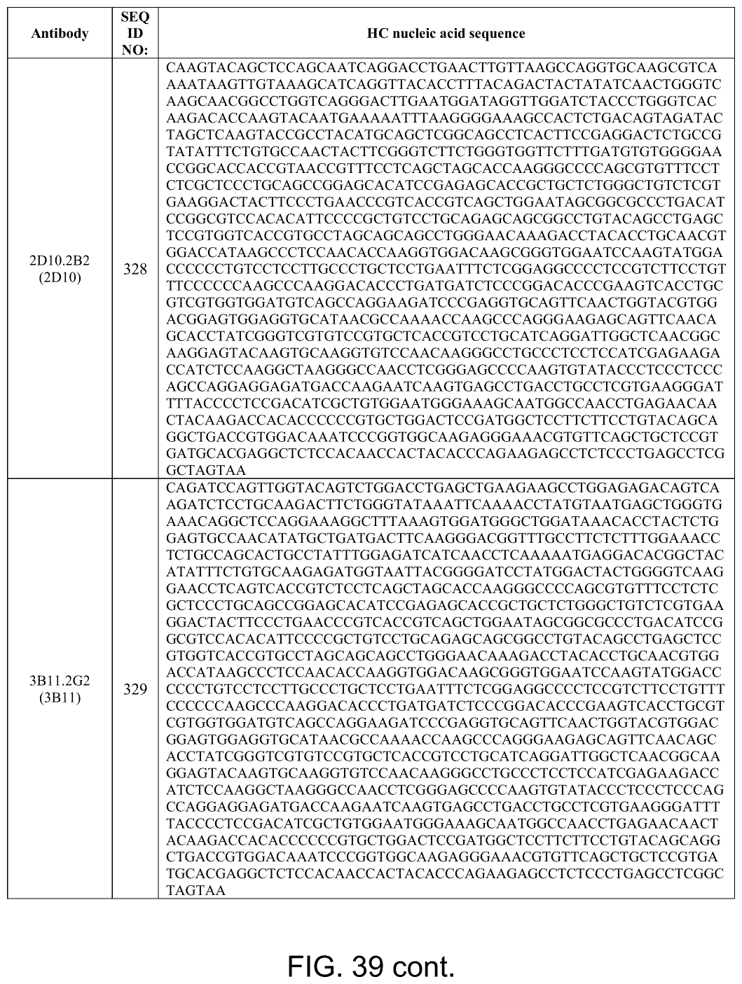

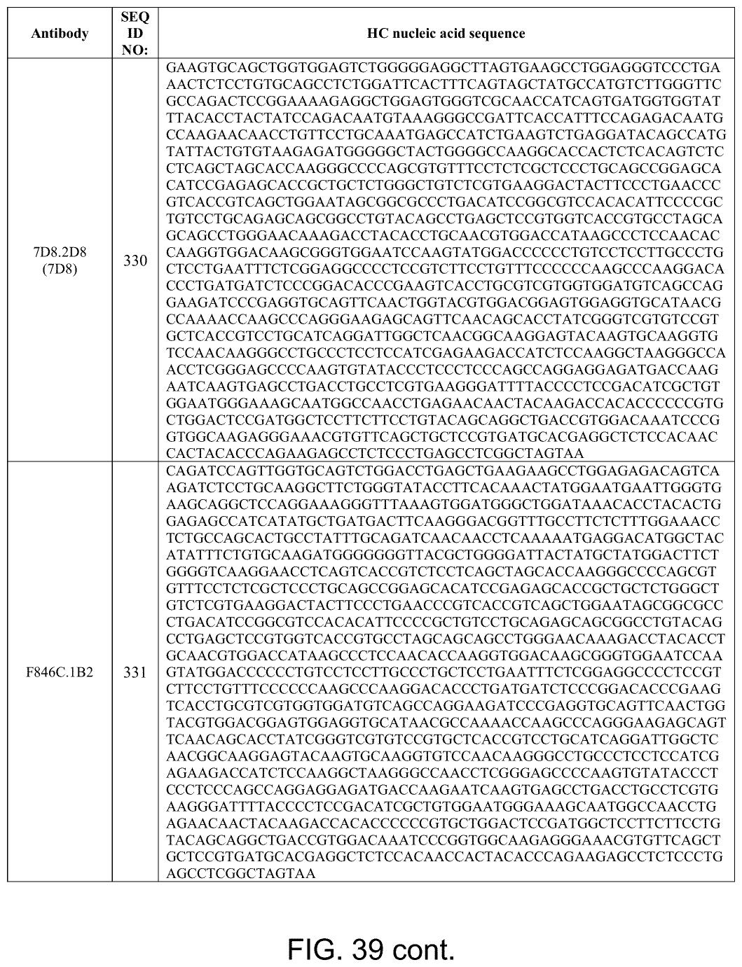

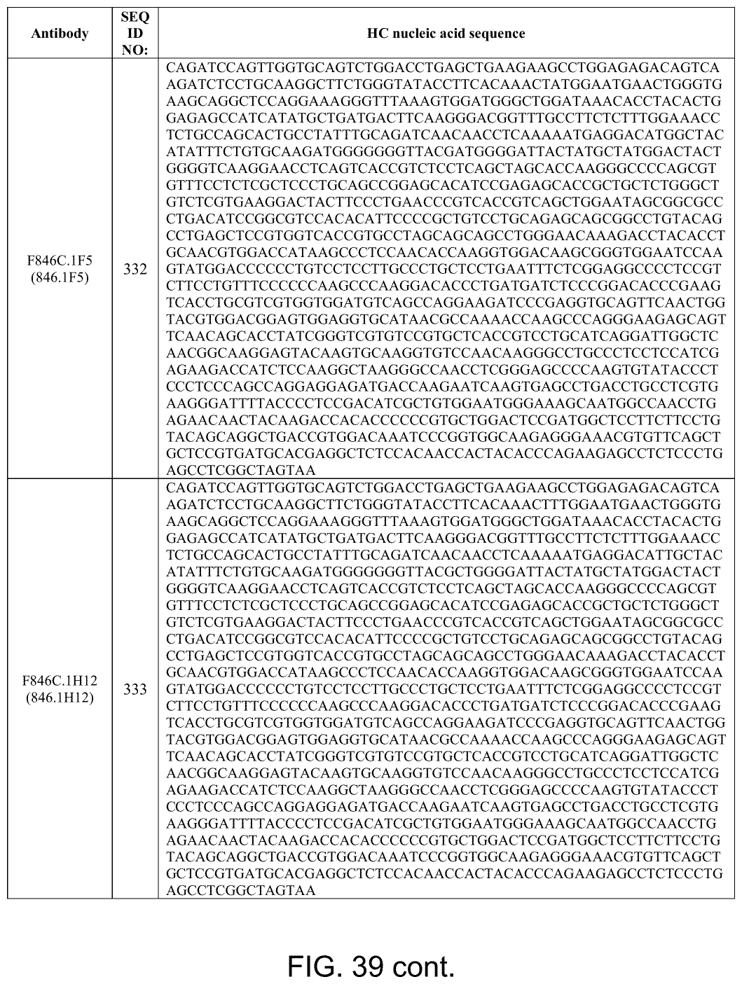

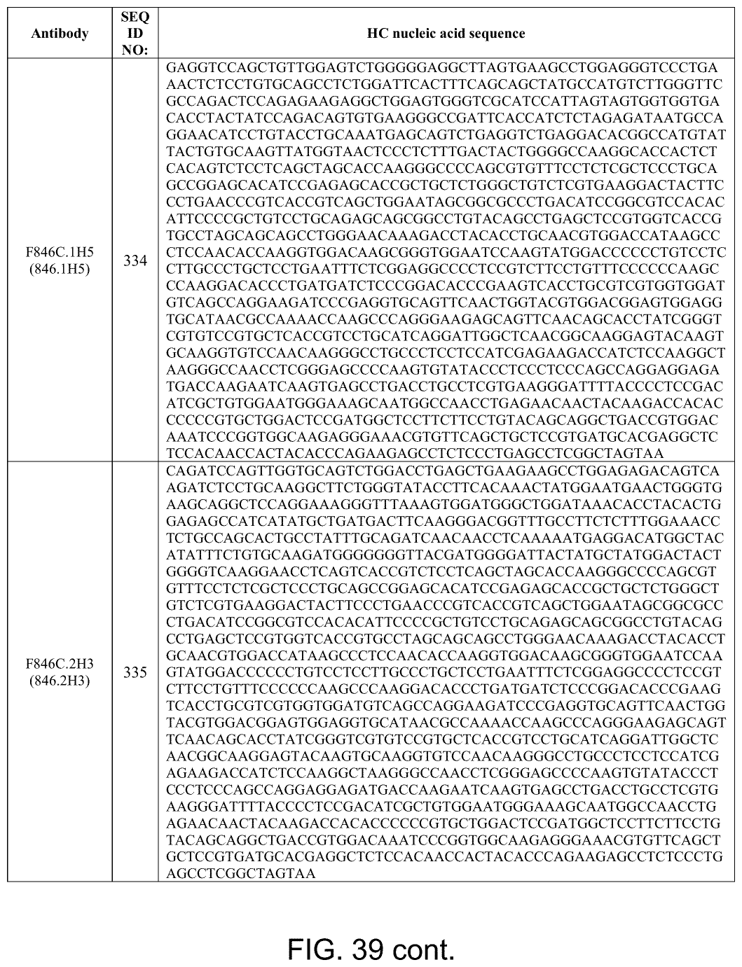

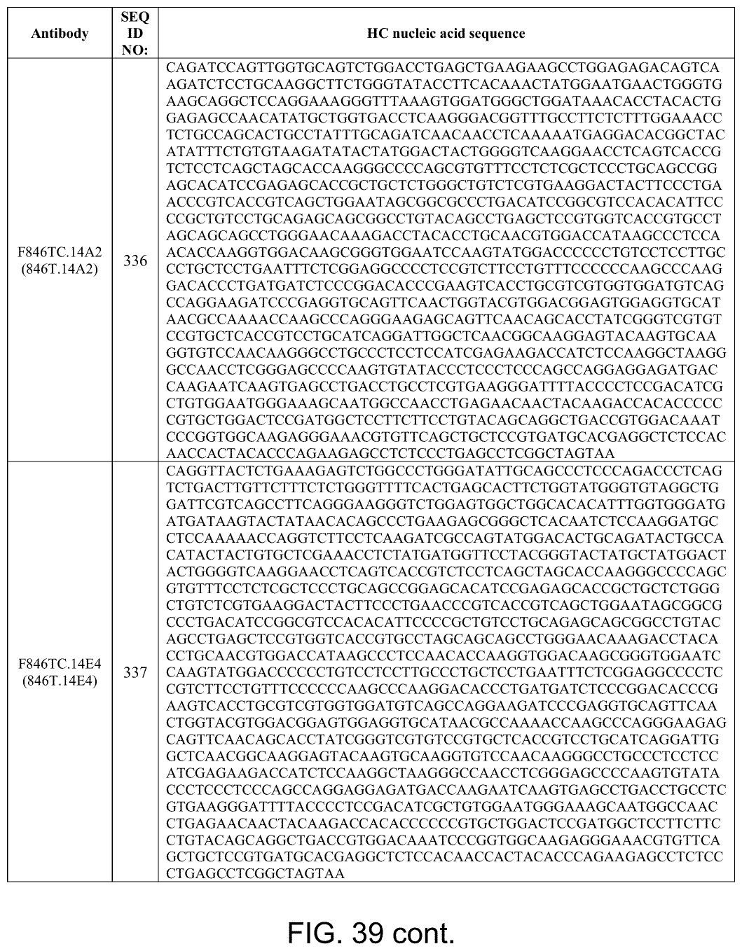

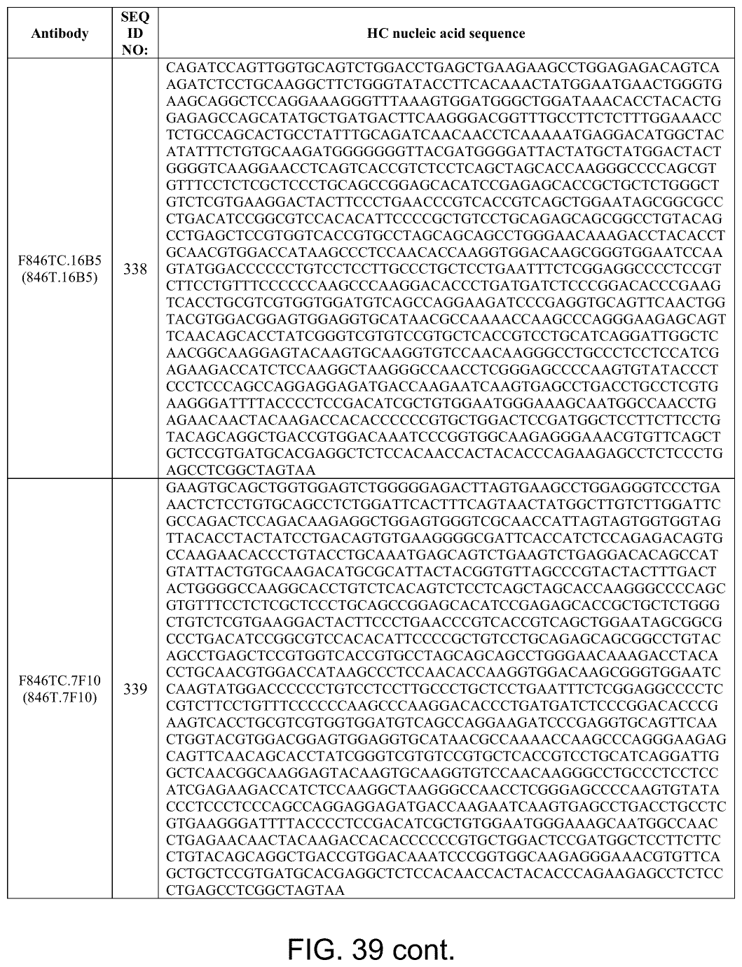

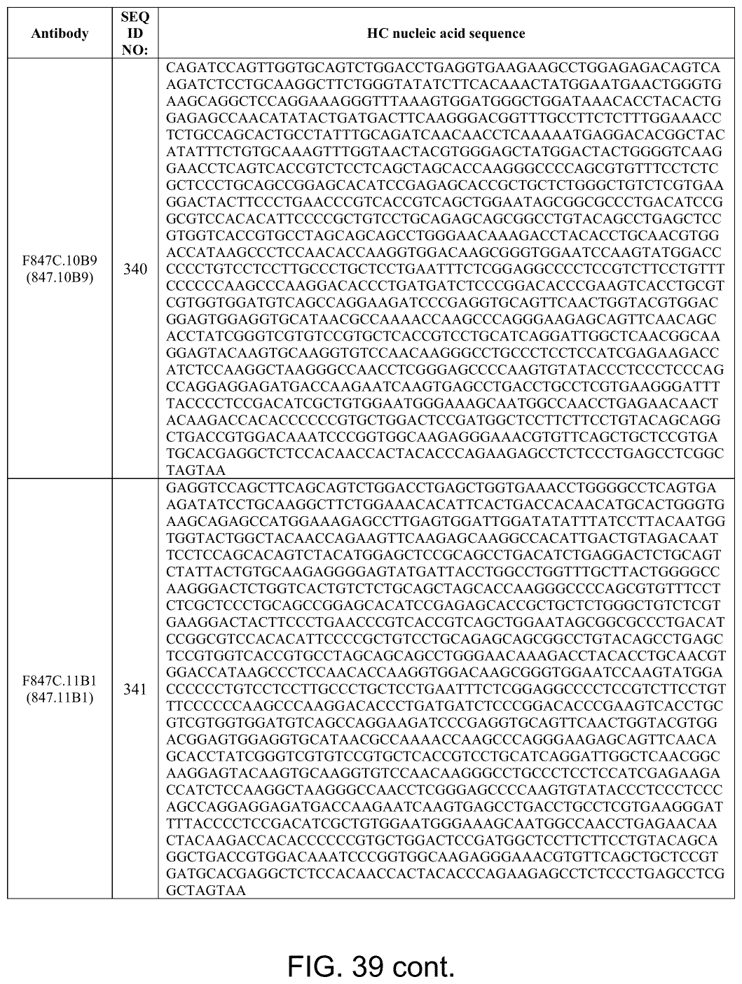

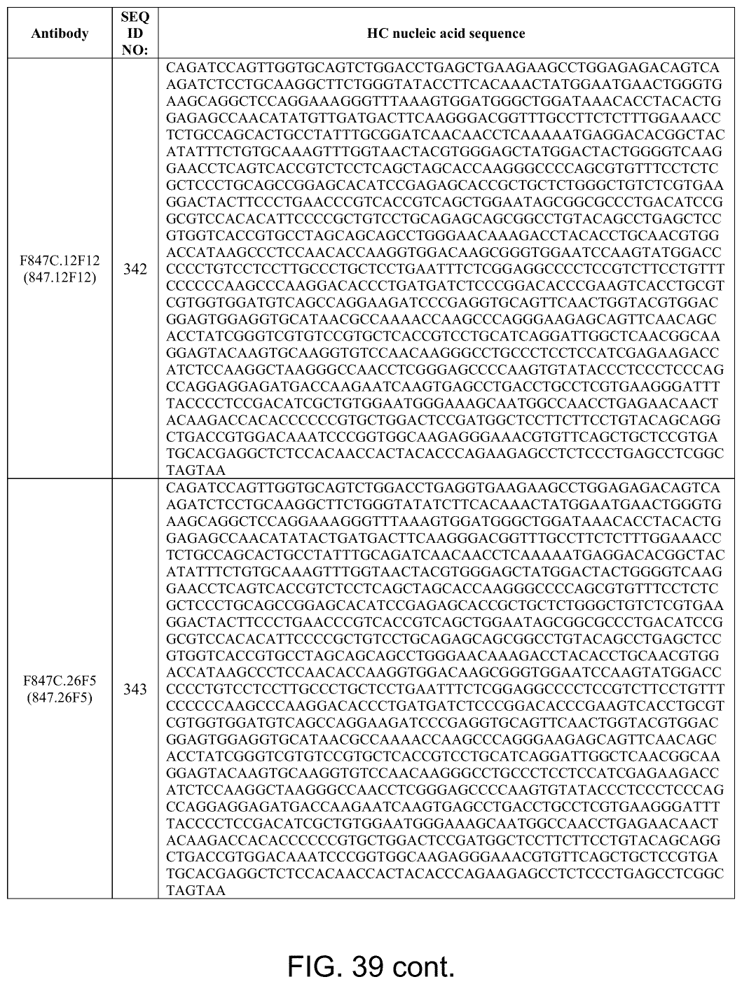

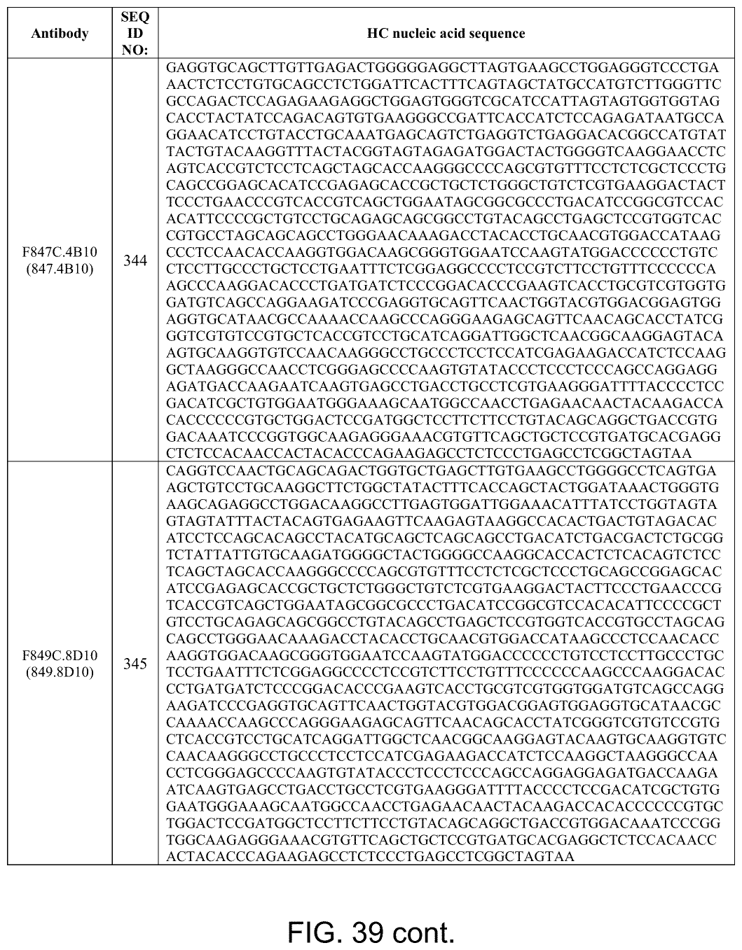

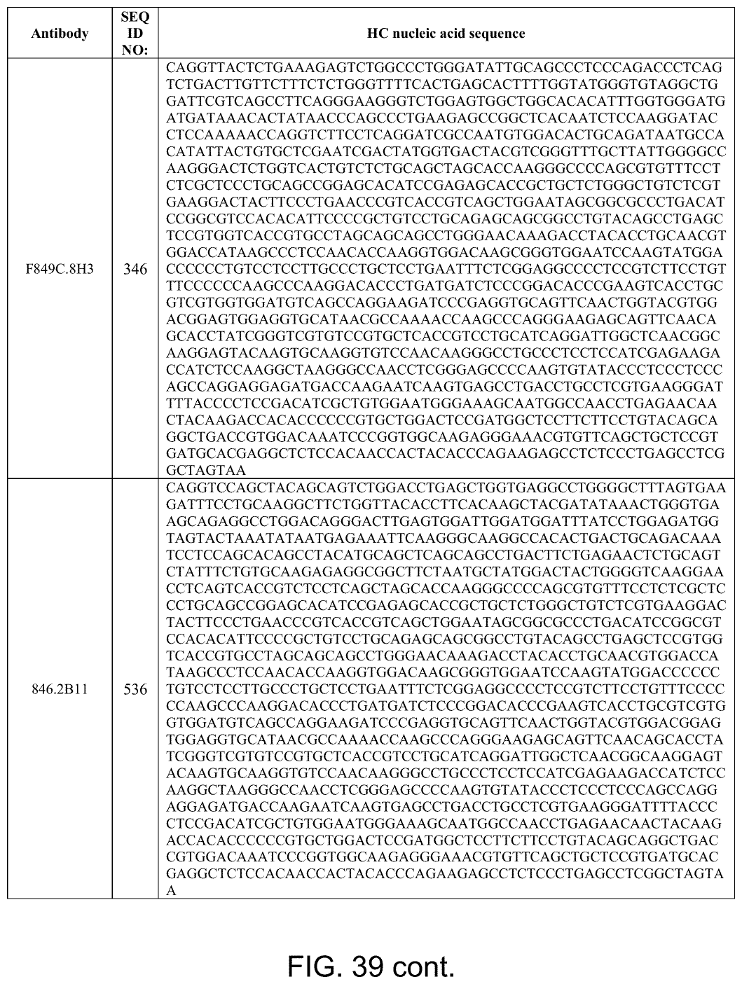

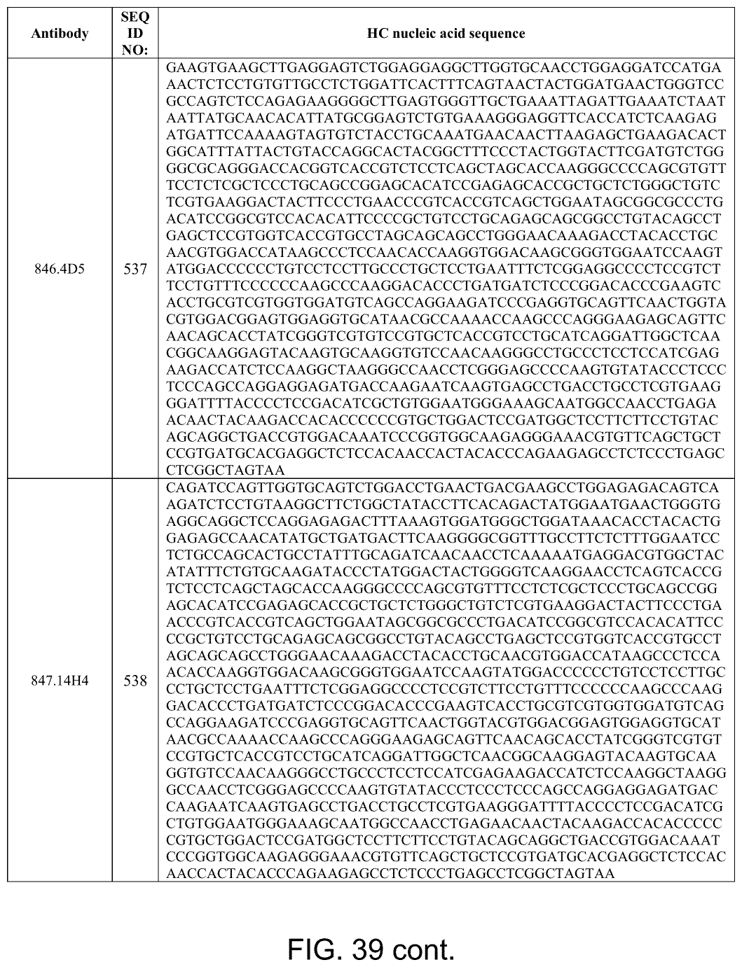

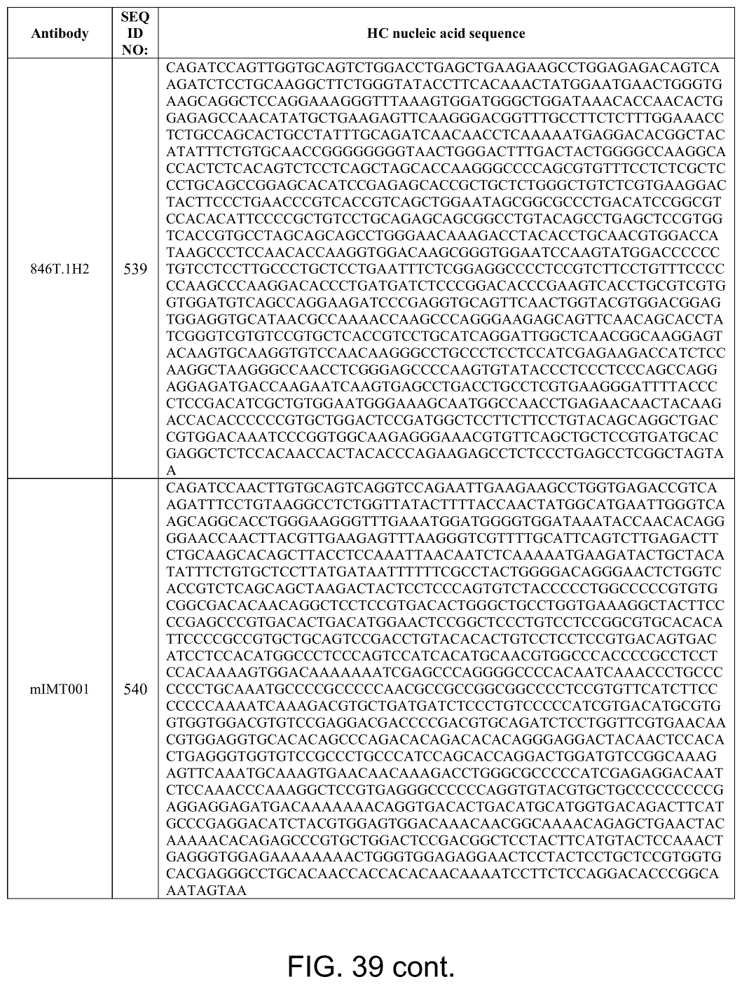

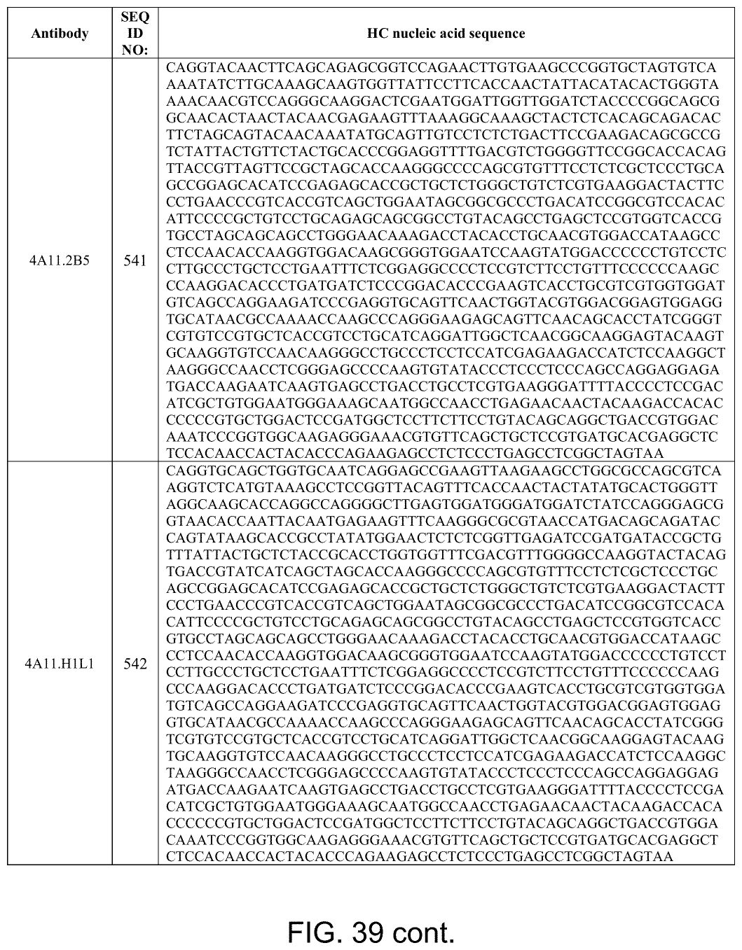

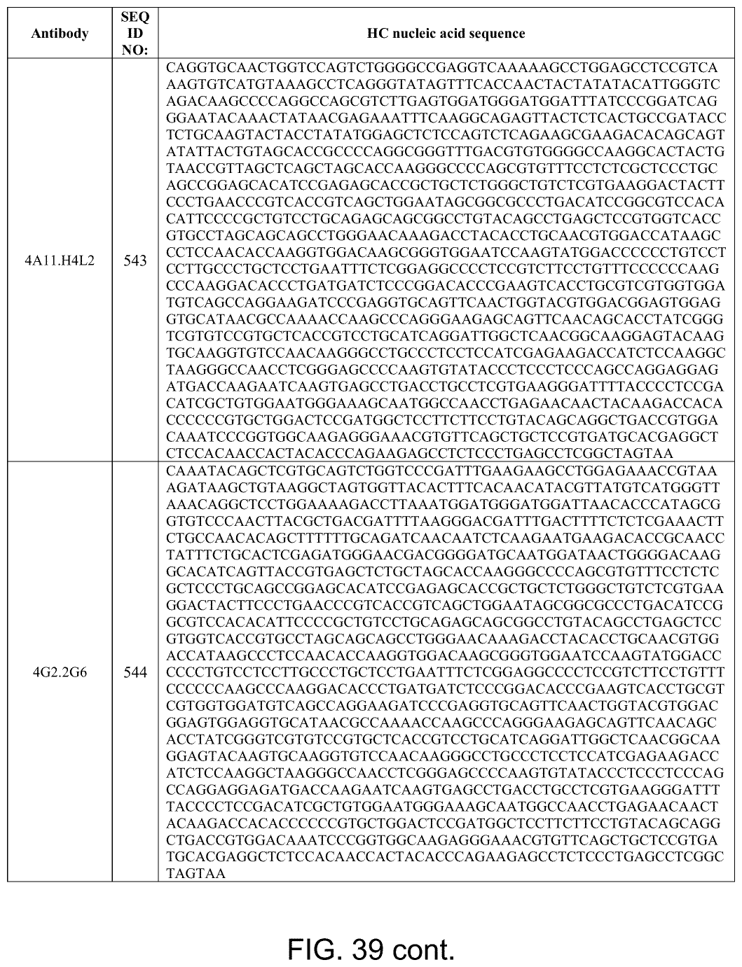

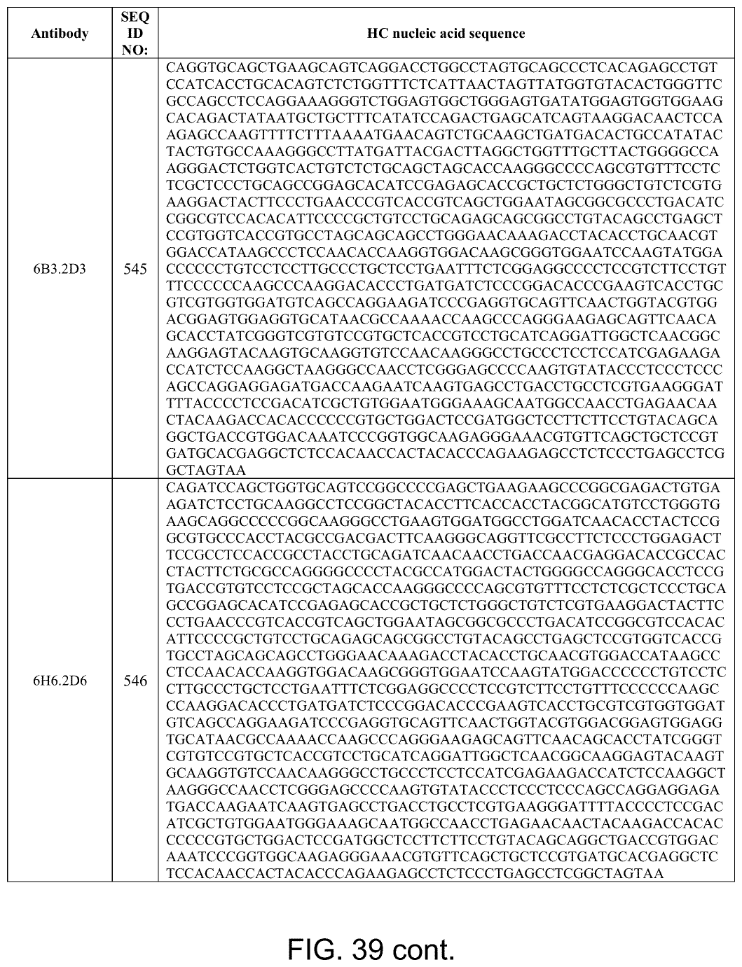

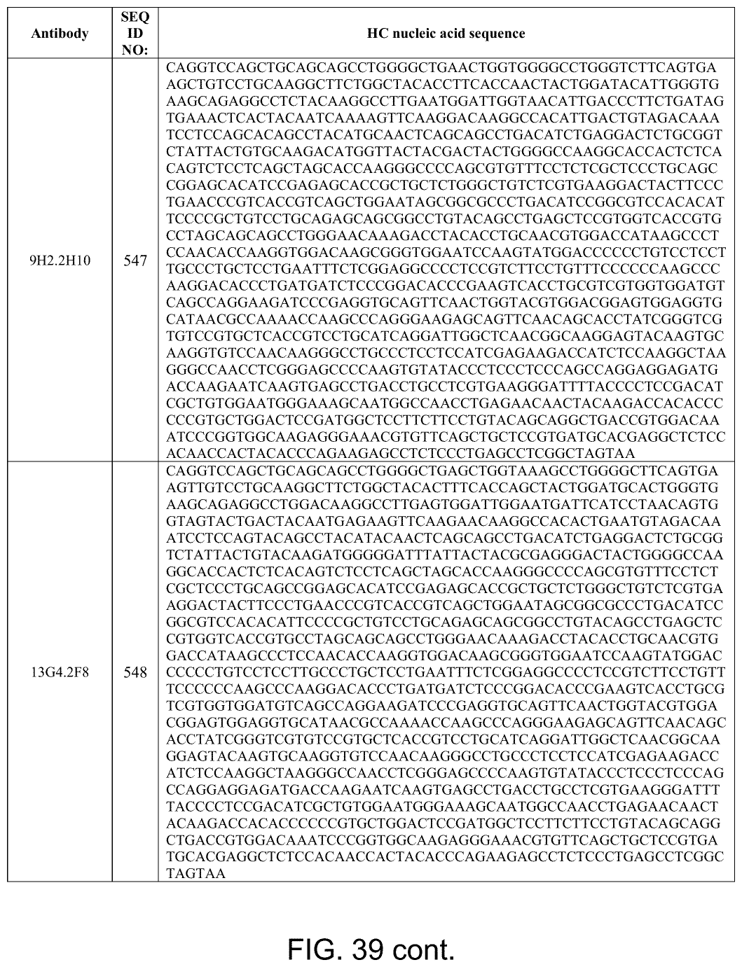

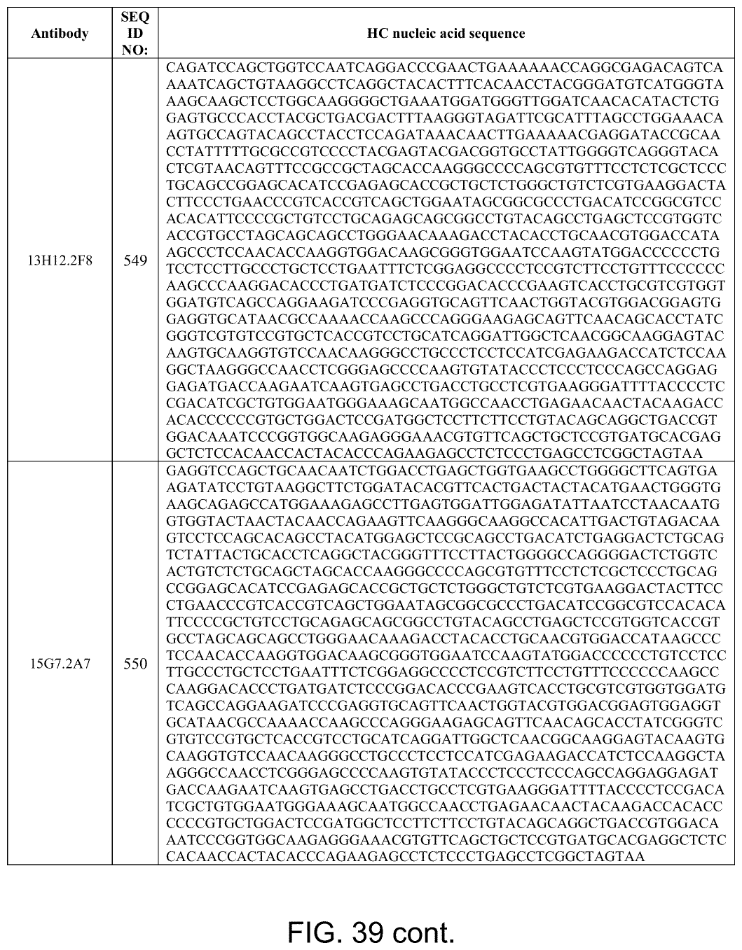

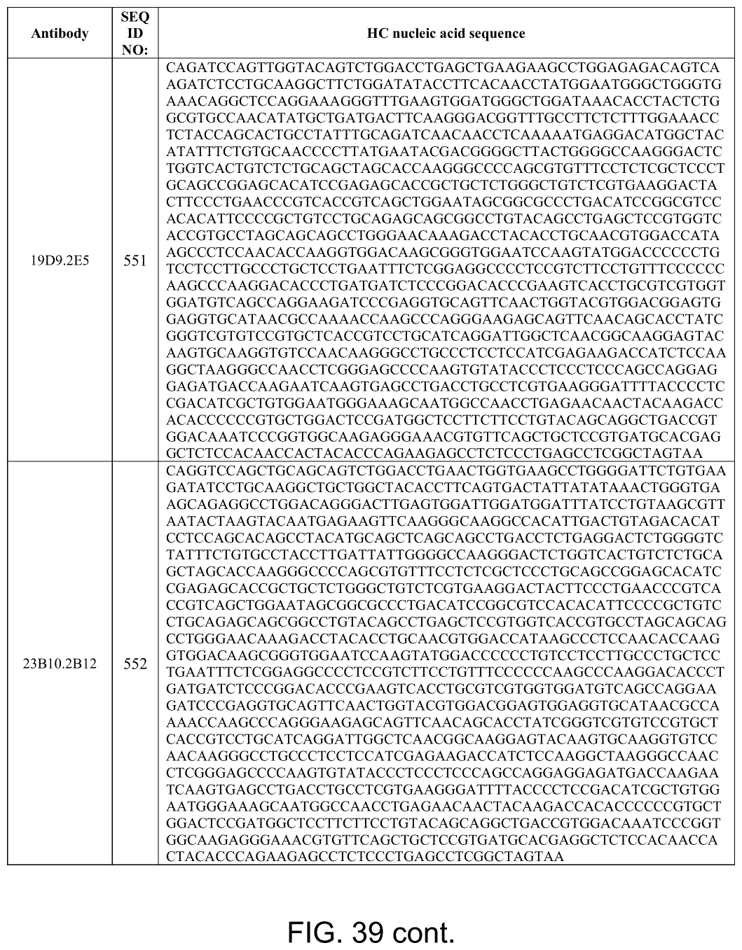

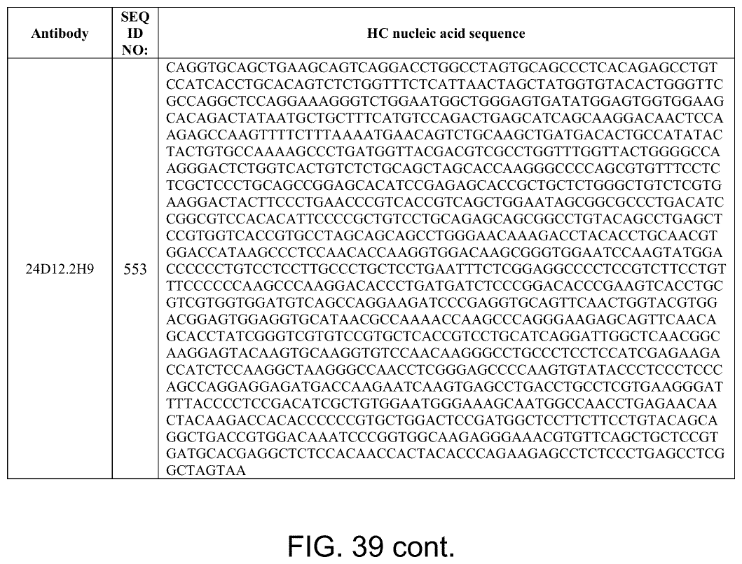

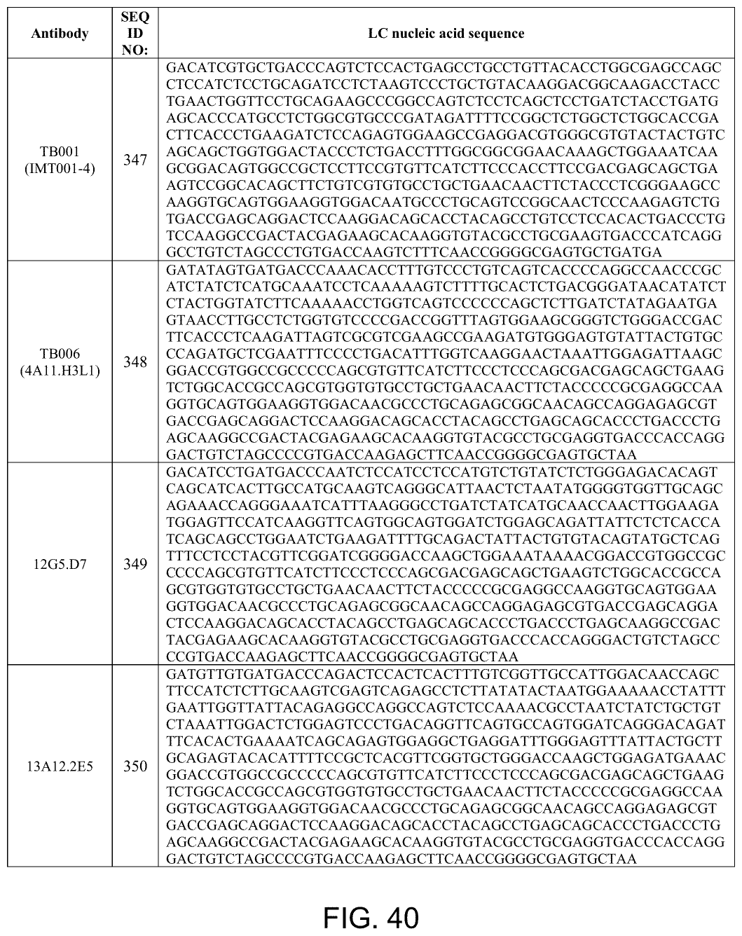

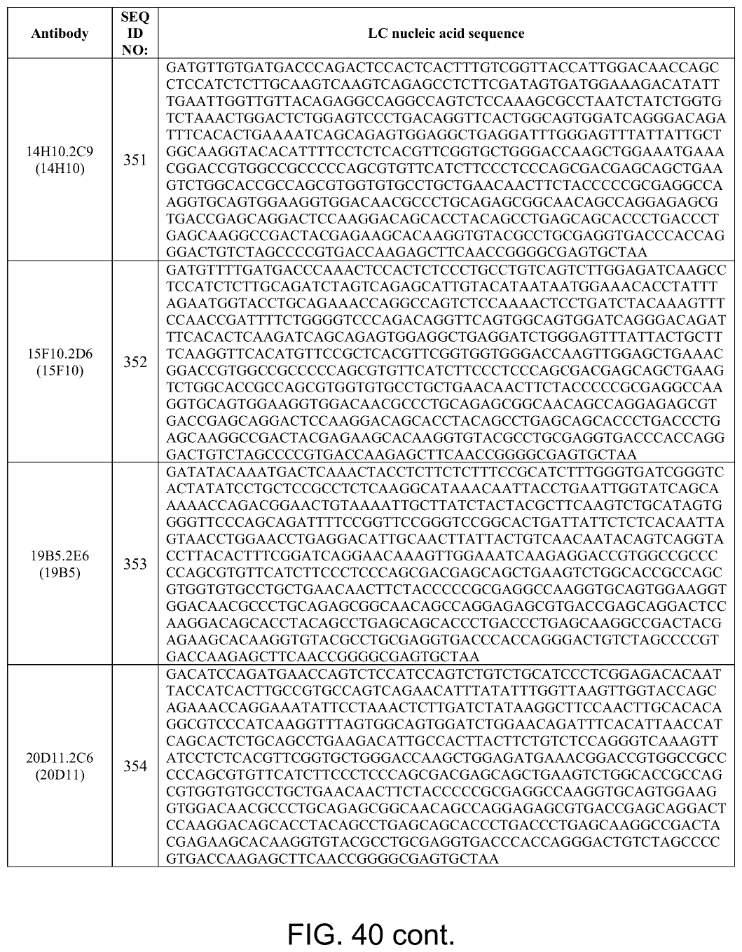

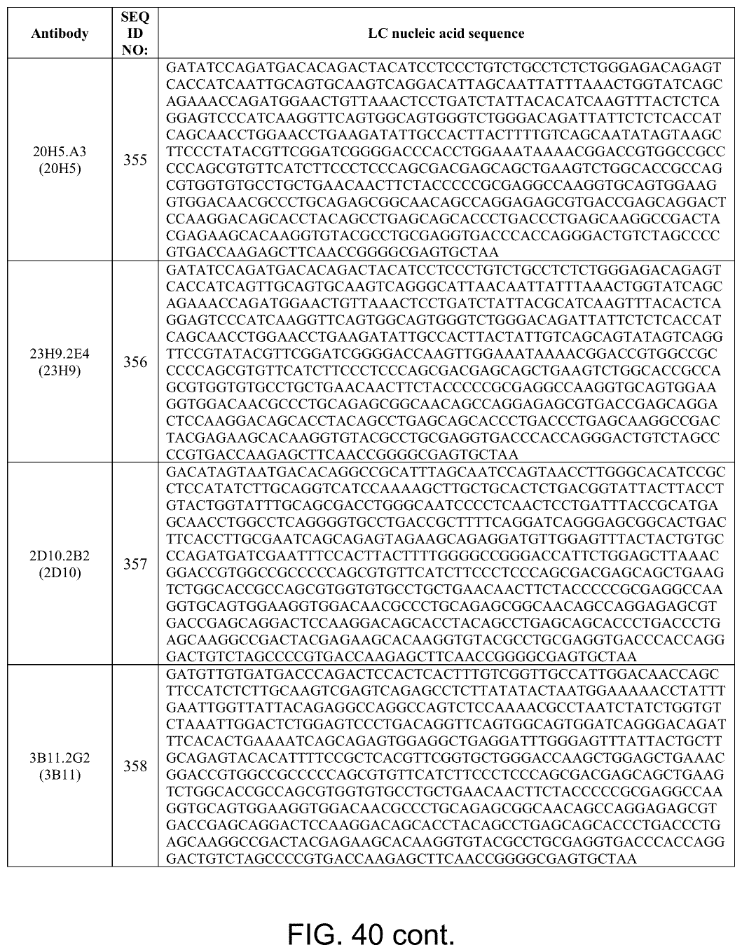

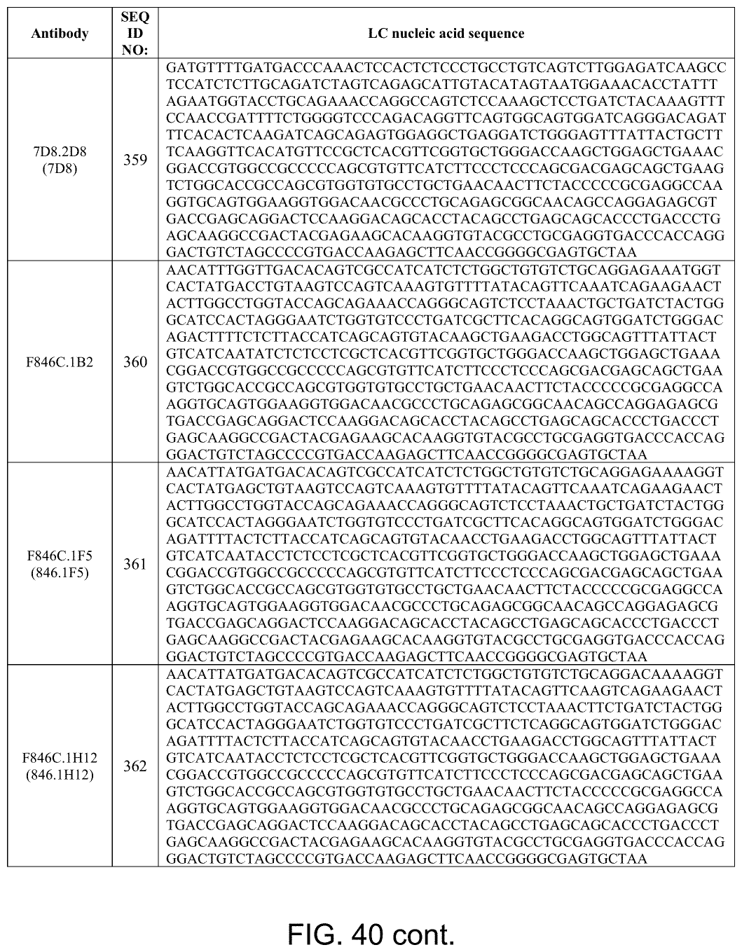

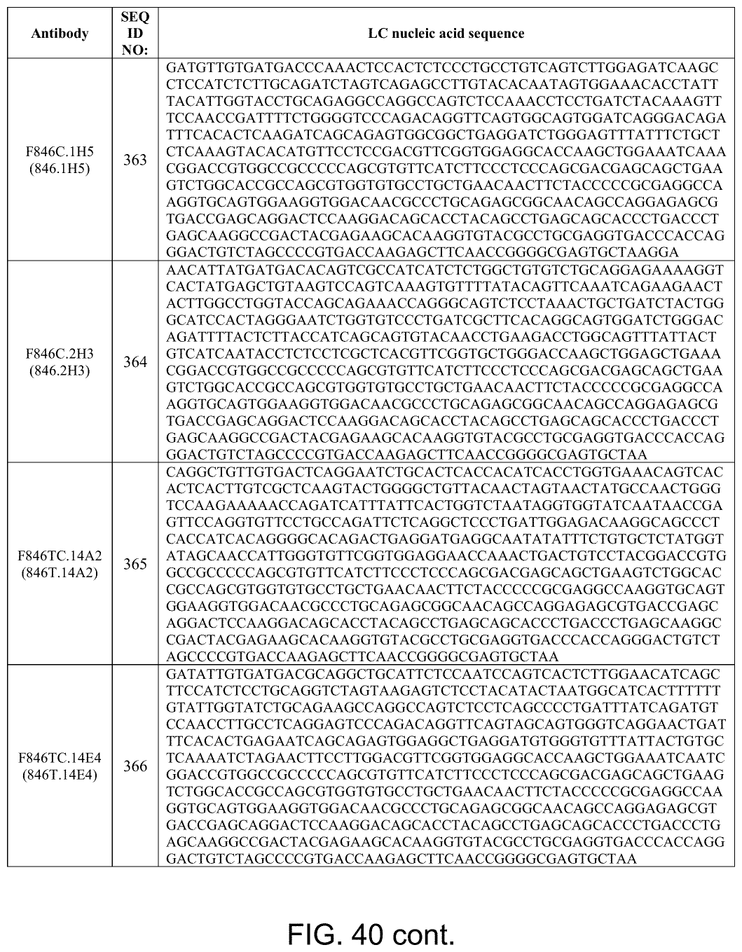

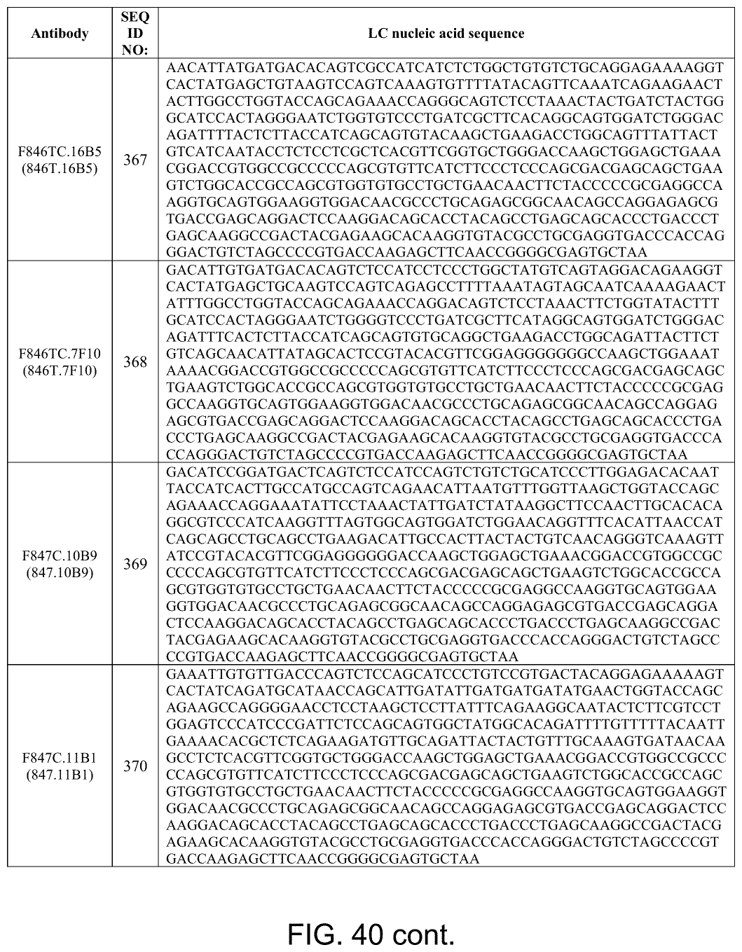

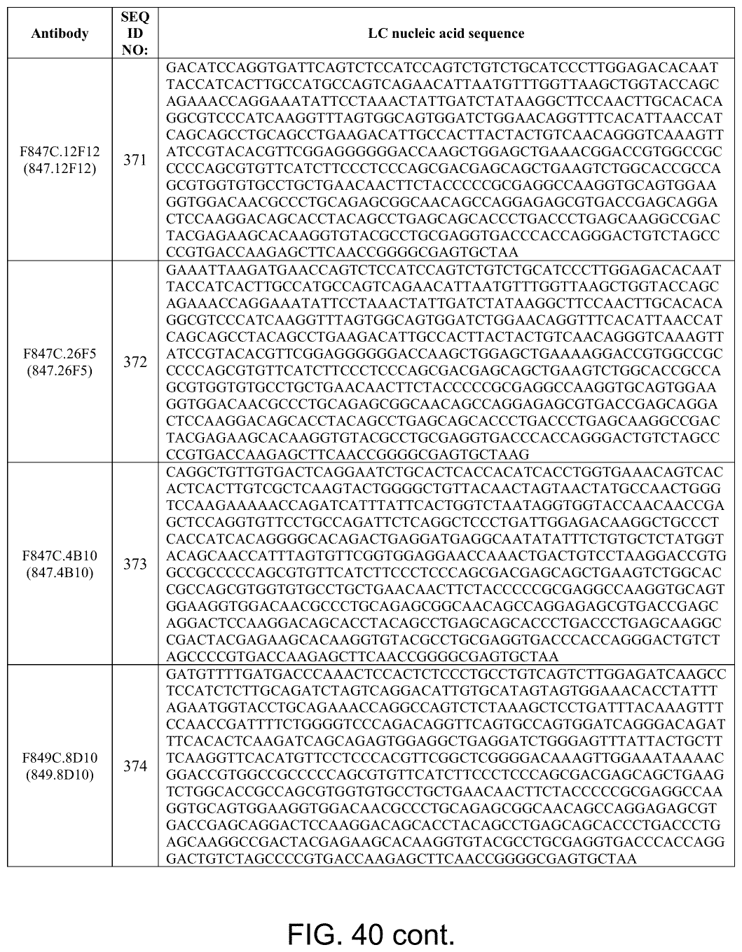

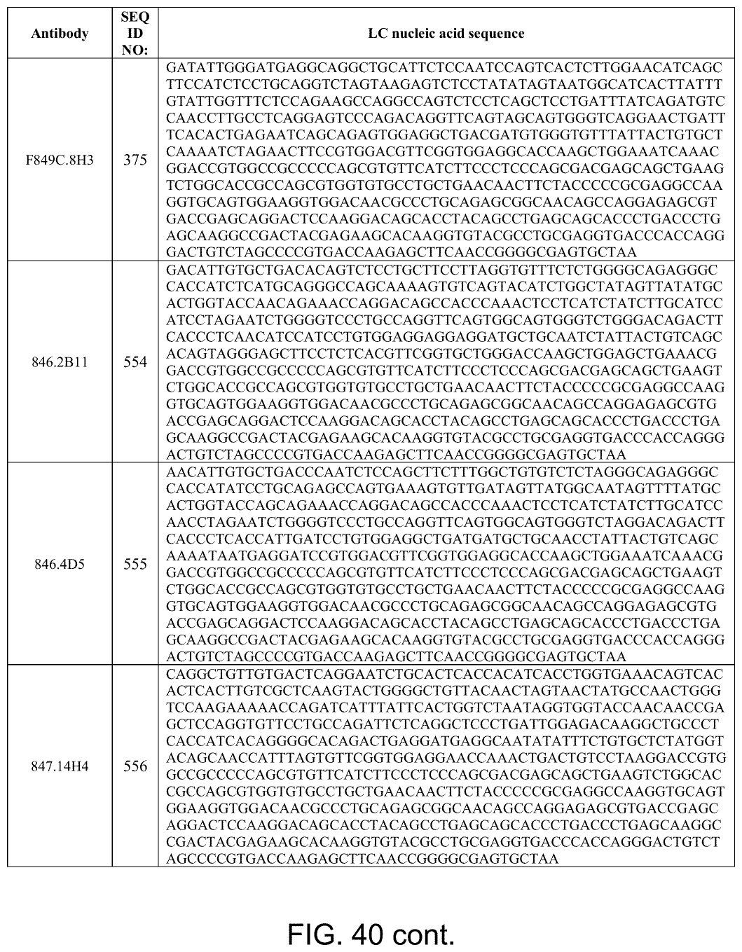

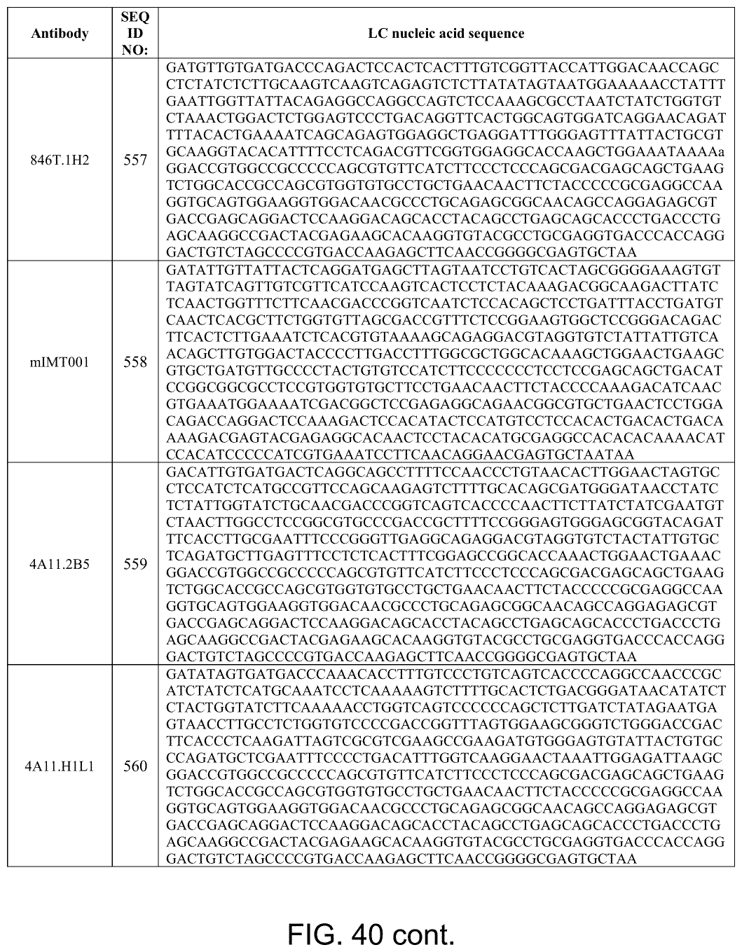

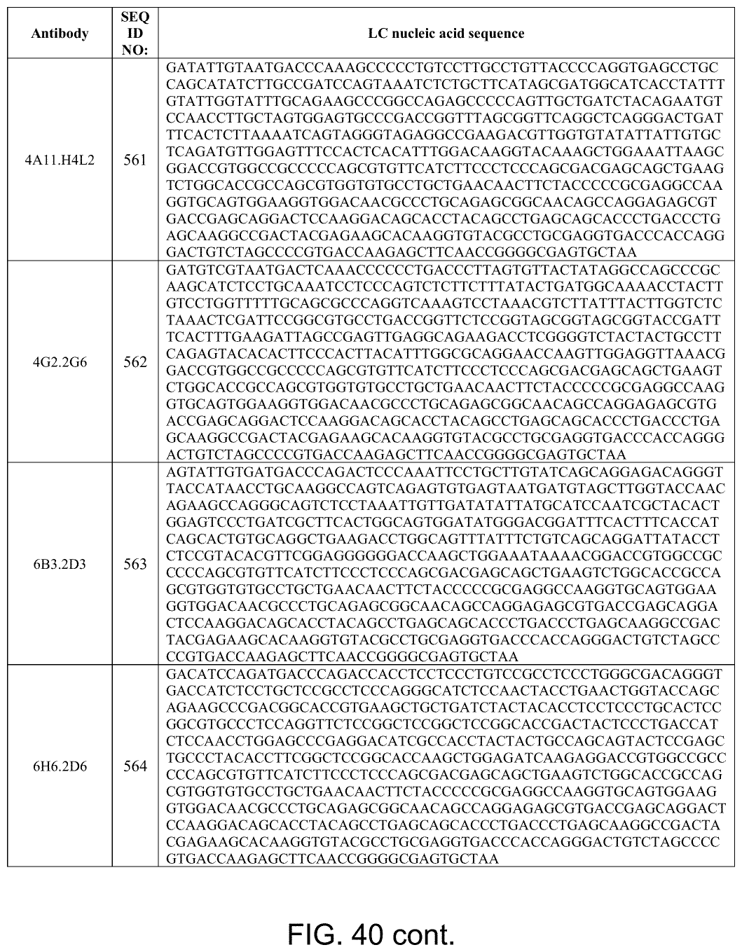

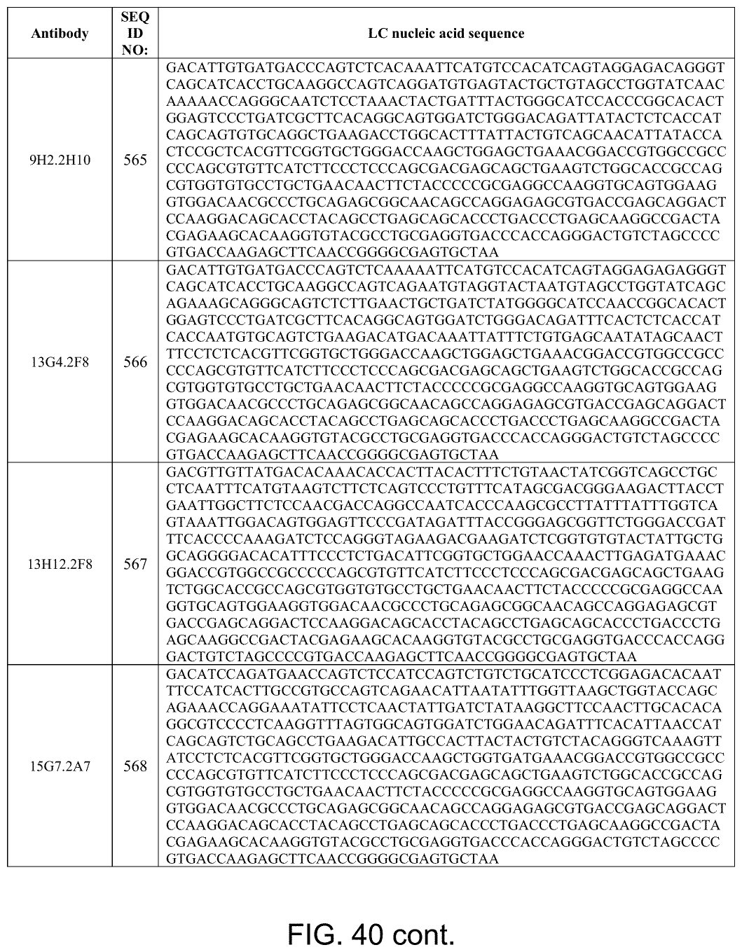

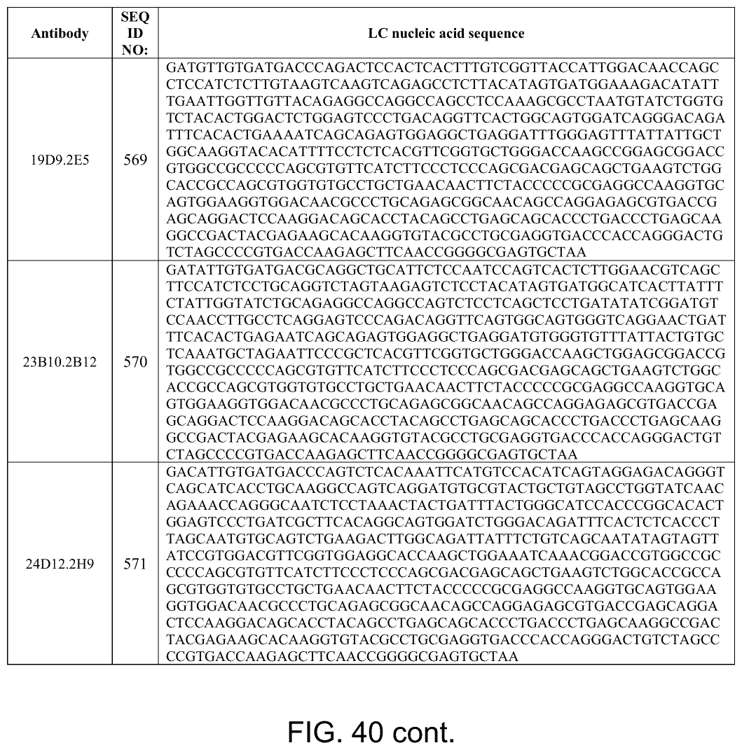

In some embodiments, nucleic acid sequences encoding for heavy chain variable regions are depicted in FIG. 37 (SEQ ID NOs: 260-288, 500-517, 812-839). In some embodiments, nucleic acid sequences encoding for light chain variable regions are depicted in FIG. 38 (SEQ ID NOs: 289-317, 518-535, 840-867). In some embodiments, nucleic acid sequences encoding for heavy chains are depicted in FIG. 39 (SEQ ID NOs: 318-346, 536-553). In some embodiments, nucleic acid sequences encoding for light chains are depicted in FIG. 40 (SEQ ID NOs: 347-375, 554-571).

Any one of the anti-Gal3 antibodies or binding fragments thereof described herein can be prepared by recombinant DNA technology, synthetic chemistry techniques, or a combination thereof. For instance, sequences encoding the desired components of the anti-Gal3 antibodies, including light chain CDRs and heavy chain CDRs are typically assembled cloned into an expression vector using standard molecular techniques known in the art. These sequences may be assembled from other vectors encoding the desired protein sequence, from PCR-generated fragments using respective template nucleic acids, or by assembly of synthetic oligonucleotides encoding the desired sequences. Expression systems can be created by transfecting a suitable cell with an expressing vector which comprises an anti-Gal3 antibody of interest or binding fragment thereof.

Nucleotide sequences corresponding to various regions of light or heavy chains of an existing antibody can be readily obtained and sequenced using convention techniques including but not limited to hybridization, PCR, and DNA sequencing. Hybridoma cells that produce monoclonal antibodies serve as a preferred source of antibody nucleotide sequences. A vast number of hybridoma cells producing an array of monoclonal antibodies may be obtained from public or private repositories. The largest depository agent is American Type Culture Collection, which offers a diverse collection of well-characterized hybridoma cell lines. Alternatively, antibody nucleotides can be obtained from immunized or non-immunized rodents or humans, and form organs such as spleen and peripheral blood lymphocytes. Specific techniques applicable for extracting and synthesizing antibody nucleotides are described in Orlandi et al. (1989) Proc. Natl. Acad. Sci. U.S.A. 86: 3833-3837; Larrick et al. (1989) Biochem. Biophys. Res. Commun. 160:1250-1255; Sastry et al. (1989) Proc. Natl. Acad. Sci., U.S.A. 86: 5728-5732; and U.S. Pat. No. 5,969,108.

Polynucleotides encoding anti-Gal3 antibodies or binding fragments thereof can also be modified, for example, by substituting the coding sequence for human heavy and light chain constant regions in place of the homologous non-human sequences. In that manner, chimeric antibodies are prepared that retain the binding specificity of the original anti-Gal3 antibody or binding fragment thereof.

### Host Cells

In some embodiments, the present disclosure provides host cells expressing any one of the anti-Gal3 antibodies or binding fragments thereof disclosed herein. A subject host cell typically comprises a nucleic acid encoding any one of the anti-Gal3 antibodies or binding fragments thereof disclosed herein.

The disclosure provides host cells transfected with the polynucleotides, vectors, or a library of the vectors described above. The vectors can be introduced into a suitable prokaryotic or eukaryotic cell by any of a number of appropriate means, including electroporation, microprojectile bombardment; lipofection, infection (where the vector is coupled to an infectious agent), transfection employing calcium chloride, rubidium chloride, calcium phosphate, DEAE-dextran, or other substances. The choice of the means for introducing vectors will often depend on features of the host cell.

For most animal cells, any of the above-mentioned methods is suitable for vector delivery. Preferred animal cells are vertebrate cells, preferably mammalian cells, capable of expressing exogenously introduced gene products in large quantity, e.g. at the milligram level. Non-limiting examples of preferred cells are NIH3T3 cells, COS, HeLa, and CHO cells.

Once introduced into a suitable host cell, expression of the anti-Gal3 antibodies or binding fragments thereof can be determined using any nucleic acid or protein assay known in the art. For example, the presence of transcribed mRNA of light chain CDRs or heavy chain CDRs, or the anti-Gal3 antibody or binding fragment thereof can be detected and/or quantified by conventional hybridization assays (e.g. Northern blot analysis), amplification procedures (e.g. RT-PCR), SAGE (U.S. Pat. No. 5,695,937), and array-based technologies (see e.g. U.S. Pat. Nos. 5,405,783, 5,412,087 and 5,445,934), using probes complementary to any region of a polynucleotide that encodes the anti-Gal3 antibody or binding fragment thereof.

Expression of the vector can also be determined by examining the expressed anti-Gal3 antibody or binding fragment thereof. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

### Payload

In some embodiments, any anti-Gal3 antibody disclosed herein further comprises a payload. In some cases, the payload comprises a small molecule, a protein or functional fragment thereof, a peptide, or a nucleic acid polymer.

In some cases, the number of payloads conjugated to the anti-Gal3 antibody (e.g., the drug-to-antibody ratio or DAR) is about 1:1, one payload to one anti-Gal3 antibody. In some cases, the ratio of the payloads to the anti-Gal3 antibody is about 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, or 20:1. In some cases, the ratio of the payloads to the anti-Gal3 antibody is about 2:1. In some cases, the ratio of the payloads to the anti-Gal3 antibody is about 3:1. In some cases, the ratio of the payloads to the anti-Gal3 antibody is about 4:1. In some cases, the ratio of the payloads to the anti-Gal3 antibody is about 6:1. In some cases, the ratio of the payloads to the anti-Gal3 antibody is about 8:1. In some cases, the ratio of the payloads to the anti-Gal3 antibody is about 12:1.

In some embodiment, the payload is a small molecule. In some instances, the small molecule is a cytotoxic payload. Exemplary cytotoxic payloads include, but are not limited to, microtubule disrupting agents, DNA modifying agents, or Akt inhibitors.

In some embodiments, the payload comprises a microtubule disrupting agent. Exemplary microtubule disrupting agents include, but are not limited to, 2-methoxyestradiol, auristatin, chalcones, colchicine, combretastatin, cryptophycin, dictyostatin, discodermolide, dolastain, eleutherobin, epothilone, halichondrin, laulimalide, maytansine, noscapinoid, paclitaxel, peloruside, phomopsin, podophyllotoxin, rhizoxin, spongistatin, taxane, tubulysin, vinca alkaloid, vinorelbine, or derivatives or analogs thereof.

In some embodiments, the maytansine is a maytansinoid. In some embodiments, the maytansinoid is DM1, DM4, or ansamitocin. In some embodiments, the maytansinoid is DM1. In some embodiments, the maytansinoid is DM4. In some embodiments, the maytansinoid is ansamitocin. In some embodiments, the maytansinoid is a maytansionid derivative or analog such as described in U.S. Pat. Nos. 5,208,020, 5,416,064, 7,276,497, and 6,716,821 or U.S. Publication Nos. 2013029900 and US20130323268.

In some embodiments, the payload is a dolastatin, or a derivative or analog thereof. In some embodiments, the dolastatin is dolastatin 10 or dolastatin 15, or derivatives or analogs thereof. In some embodiments, the dolastatin 10 analog is auristatin, soblidotin, symplostatin 1, or symplostatin 3. In some embodiments, the dolastatin 15 analog is cemadotin or tasidotin.

In some embodiments, the dolastatin 10 analog is auristatin or an auristatin derivative. In some embodiments, the auristatin or auristatin derivative is auristatin E (AE), auristatin F (AF), auristatin E5-benzoylvaleric acid ester (AEVB), monomethyl auristatin E (MMAE), monomethyl auristatin F (MMAF), or monomethyl auristatin D (MMAD), auristatin PE, or auristatin PYE. In some embodiments, the auristatin derivative is monomethyl auristatin E (MMAE). In some embodiments, the auristatin derivative is monomethyl auristatin F (MMAF). In some embodiments, the auristatin is an auristatin derivative or analog such as described in U.S. Pat. Nos. 6,884,869, 7,659,241, 7,498,298, 7,964,566, 7,750,116, 8,288,352, 8,703,714, and 8,871,720.

In some embodiments, the payload comprises a DNA modifying agent. In some embodiments, the DNA modifying agent comprises DNA cleavers, DNA intercalators, DNA transcription inhibitors, or DNA cross-linkers. In some instances, the DNA cleaver comprises bleomycin A2, calicheamicin, or derivatives or analogs thereof. In some instances, the DNA intercalator comprises doxorubicin, epirubicin, PNU-159682, duocarmycin, pyrrolobenzodiazepine, oligomycin C, daunorubicin, valrubicin, topotecan, or derivatives or analogs thereof. In some instances, the DNA transcription inhibitor comprises dactinomycin. In some instances, the DNA cross-linker comprises mitomycin C.

In some embodiments, the DNA modifying agent comprises amsacrine, anthracycline, camptothecin, doxorubicin, duocarmycin, enediyne, etoposide, indolinobenzodiazepine, netropsin, teniposide, or derivatives or analogs thereof.

In some embodiments, the anthracycline is doxorubicin, daunorubicin, epirubicin, idarubicin, mitomycin-C, dactinomycin, mithramycin, nemorubicin, pixantrone, sabarubicin, or valrubicin.

In some embodiments, the analog of camptothecin is topotecan, irinotecan, silatecan, cositecan, exatecan, lurtotecan, gimatecan, belotecan, rubitecan, or SN-38.

In some embodiments, the duocarmycin is duocarmycin A, duocarmycin B1, duocarmycin B2, duocarmycin C1, duocarmycin C2, duocarmycin D, duocarmycin SA, or CC-1065. In some embodiments, the enediyne is a calicheamicin, esperamicin, or dynemicin A.

In some embodiments, the pyrrolobenzodiazepine is anthramycin, abbeymycin, chicamycin, DC-81, mazethramycin, neothramycins A, neothramycin B, porothramycin, prothracarcin, sibanomicin (DC-102), sibiromycin, or tomaymycin. In some embodiments, the pyrrolobenzodiazepine is a tomaymycin derivative, such as described in U.S. Pat. Nos. 8,404,678 and 8,163,736. In some embodiments, the pyrrolobenzodiazepine is such as described in U.S. Pat. Nos. 8,426,402, 8,802,667, 8,809,320, 6,562,806, 6,608,192, 7,704,924, 7,067,511, 7,612,062, 7,244,724, 7,528,126, 7,049,311, 8,633,185, 8,501,934, and 8,697,688 and U.S. Publication No. US20140294868.

In some embodiments, the pyrrolobenzodiazepine is a pyrrolobenzodiazepine dimer. In some embodiments, the PBD dimer is a symmetric dimer. Examples of symmetric PBD dimers include, but are not limited to, SJG-136 (SG-2000), ZC-423 (SG2285), SJG-720, SJG-738, ZC-207 (SG2202), and DSB-120. In some embodiments, the PBD dimer is an unsymmetrical dimer. Examples of unsymmetrical PBD dimers include, but are not limited to, SJG-136 derivatives such as described in U.S. Pat. Nos. 8,697,688 and 9,242,013 and U.S. Publication No. 20140286970.

In some embodiments, the payload comprises an Akt inhibitor. In some cases, the Akt inhibitor comprises ipatasertib (GDC-0068) or derivatives thereof.

In some embodiments, the payload comprises a polymerase inhibitor, including, but not limited to polymerase II inhibitors such as a-amanitin, and poly(ADP-ribose) polymerase (PARP) inhibitors. Exemplary PARP inhibitors include, but are not limited to Iniparib (BSI 201), Talazoparib (BMN-673), Olaparib (AZD-2281), Olaparib, Rucaparib (AG014699, PF-01367338), Veliparib (ABT-888), CEP 9722, MK 4827, BGB-290, or 3-aminobenzamide.

In some embodiments, the payload comprises a detectable moiety. As used herein, a “detectable moiety” may comprise an atom, molecule, or compound that is useful in diagnosing, detecting or visualizing a location and/or quantity of a target molecule, cell, tissue, organ, and the like. Detectable moieties that can be used in accordance with the embodiments herein include, but are not limited to, radioactive substances (e.g. radioisotopes, radionuclides, radiolabels or radiotracers), dyes, contrast agents, fluorescent compounds or molecules, bioluminescent compounds or molecules, enzyme and enhancing agents (e.g. paramagnetic ions), or specific binding moieties such as streptavidin, avidin, or biotin. In addition, some nanoparticles, for example quantum dots or metal nanoparticles can be suitable for use as a detectable moiety.

Exemplary radioactive substances that can be used as detectable moieties in accordance with the embodiments herein include, but are not limited to, ¹⁸F, ¹⁸F-FAC, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁵Sc, ⁷⁷As, ⁸⁶Y ⁹⁰Y, ⁸⁹Sr, ⁸⁹Zr, ⁹⁴Tc, ⁹⁴Tc, ⁹⁹mTc, ⁹⁹Mo, ¹⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸Gd, ¹⁶¹T, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴I, ¹⁹⁸Au, ¹⁹⁹Au, ²¹¹At, ²¹¹Pb, ²¹²Bi, ²¹²Pb, ²¹³Bi, ²²³Ra and ²²⁵Ac. Exemplary paramagnetic ions substances that can be used as detectable markers include, but are not limited to ions of transition and lanthanide metals (e.g. metals having atomic numbers of 6 to 9, 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the detectable marker is a radioactive metal or paramagnetic ion, in some embodiments, the marker can be reacted with a reagent having a long tail with one or more chelating groups attached to the long tail for binding these ions. The long tail can be a polymer such as apolylysine, polysaccharide, or other derivatized or derivatizable chain having pendant groups to which may be bound to a chelating group for binding the ions. Examples of chelating groups that may be used according to the embodiments herein include, but are not limited to, ethylenediaminetetraacetic acid (EDTA), diethylenetriaminepentaacetic acid (DTPA), DOTA, NOTA, NOGADA, NETA, deferoxamine (DfO), porphyrins, polyamines, crown ethers, bis-thiosemicarbazones, polyoximes, and like groups. The chelate can be linked to the antigen binding construct by a group which allows formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antigen binding constructs and carriers described herein. Macrocyclic chelates such as NOTA, NOGADA, DOTA, and TETA are of use with a variety of metals and radiometals including, but not limited to, radionuclides of gallium, yttrium and copper, respectively. Other ring-type chelates such as macrocyclic polyethers, which are of interest for stably binding radionuclides, such as Radium-223 for RAIT may be used. In certain embodiments, chelating moieties may be used to attach a PET imaging agent, such as an Aluminum-18F complex, to a targeting molecule for use in PET analysis.

Exemplary contrast agents that can be used as detectable moieties in accordance with the embodiments of the disclosure include, but are not limited to, barium, diatrizoate, ethiodized oil, gallium citrate, iocarmic acid, iocetamic acid, iodamide, iodipamide, iodoxamic acid, iogulamide, iohexyl, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, thallous chloride, or combinations thereof.

Bioluminescent and fluorescent compounds or molecules and dyes that can be used as detectable moieties in accordance with the embodiments of the disclosure include, but are not limited to, allophycocyanin (APC), phycoerythrin (PE), fluorescein, fluorescein isothiocyanate (FITC), OREGON GREEN™, rhodamine, Texas red, tetrarhodimine isothiocynate (TRITC), Cy3, Cy5, and the like), fluorescent markers (e.g., green fluorescent protein (GFP) and the like), autoquenched fluorescent compounds that are activated by tumor-associated proteases, enzymes (e.g., luciferase, horseradish peroxidase, alkaline phosphatase, and the like), nanoparticles, biotin, digoxigenin or combinations thereof.

Enzymes that can be used as detectable moieties in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

In some embodiments, the payload is a nanoparticle. The term “nanoparticle” refers to a microscopic particle whose size is measured in nanometers, e.g., a particle with at least one dimension less than about 100 nm. Nanoparticles can be used as detectable substances because they are small enough to scatter visible light rather than absorb it. For example, gold nanoparticles possess significant visible light extinction properties and appear deep red to black in solution. As a result, compositions comprising antigen binding constructs conjugated to nanoparticles can be used for the in vivo imaging of T-cells in a subject. At the small end of the size range, nanoparticles are often referred to as clusters. Metal, dielectric, and semiconductor nanoparticles have been formed, as well as hybrid structures (e.g. core-shell nanoparticles). Nanospheres, nanorods, and nanocups are just a few of the shapes that have been grown. Semiconductor quantum dots and nanocrystals are examples of additional types of nanoparticles. Such nanoscale particles can be used as payloads to be conjugated to any one of the anti-Gal3 antibodies disclosed herein.

In some embodiments, the payload is an antimicrobial agent, a therapeutic agent, a prodrug, a peptide, a protein, an enzyme, a lipid, a biological response modifier, a pharmaceutical agent, a lymphokine, a heterologous antibody or fragment thereof, a detectable label, a polyethylene glycol (PEG) molecule, or a combination of two or more of the agents. In some embodiments, the payload comprises a neuroactive polypeptide, for example, a neurotrophic factors, endocrine factors, growth factors, paracrine factors, hypothalamic release factors, neurotransmitter polypeptides, polypeptide agonists for a receptor expressed by a CNS cell, polypeptides involved in lysosomal storage disease or any combination thereof. In some embodiments, the payload comprises an IL-1 receptor antagonist (IL-IRa), dalargin, an interferon-β, Glial-derived neurotrophic factor (GDNF), tumor necrosis factor receptor (TNFR), nerve growth factor (NGF), brain derived neurotrophic factor (BDNF), neurotrophin-4/5, neurotrophin (NT)-3, a neurturin, neuregulin, a netrin, ciliary neurotrophic factor (CNTF), stem cell factor (SCF), a semaphorin, hepatocyte growth factor (HGF), epidermal growth factor (EGF), transforming growth factor (TGF)-cx, TGF-B, vascular endothelial growth factor (VEGF), platelet-derived growth factor (PDGF), heregulin, artemin, persephin, interleukins, granulocyte-colony stimulating factor (CSF), granulocyte-macrophage-CSF, cardiotrophin-1, hedgehogs, leukemia inhibitory factor (LIF), midkine, pleiotrophin, erythropoietin (EPO), bone morphogenetic proteins (BMPs), netrins, saposins, any fragment thereof, or any combination thereof. In some embodiments, the payload is another antibody, or a heavy and/or light chain, or any other fragment thereof.

In some embodiments, the payload comprises a heterologous antibody or fragment thereof, for example, a heterologous antibody or fragment thereof specifically binds to one or more of beta-secretase 1 (BACE1), CD20, CD25, CD52, CD33, CTLA-4, tenascin, alpha-4 (a4) integrin, IL-12, IL-23, the p40 subunit of IL-12/IL-23, amyloid-13 (AI3), Huntingtin, nerve growth factor (NGF), epidermal growth factor receptor (EGFR/HER1), human epidermal growth factor receptor 2 (HER2/neu), vascular endothelial growth factor (VEGF), TrkA, TNF-a, TNF-13, a-synuclein Tau, apolipoprotein E4 (ApoE4), prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, a neurotrophic factor and/or a neurotrophic factor receptor.

In some embodiments, the payload comprises an immunomodulatory agent. Useful immunomodulatory agents include anti-hormones that block hormone action on tumors and immunosuppressive agents that suppress cytokine production, down-regulate self-antigen expression, or mask MHC antigens. Representative anti-hormones include anti-estrogens including, for example, tamoxifen, raloxifene, aromatase inhibiting 4(5)-imidazoles, 4-hydroxytamoxifen, trioxifene, keoxifene, LY 117018, onapristone, and toremifene; and anti-androgens such as flutamide, nilutamide, bicalutamide, leuprolide, and goserelin; and anti-adrenal agents. Illustrative immunosuppressive agents include, but are not limited to 2-amino-6-aryl-5-substituted pyrimidines, azathioprine, cyclophosphamide, bromocryptine, danazol, dapsone, glutaraldehyde, anti-idiotypic antibodies for MHC antigens and MHC fragments, cyclosporin A, steroids such as glucocorticosteroids, streptokinase, or rapamycin.

In some embodiments, the payload comprises an immune modulator. Exemplary immune modulators include, but are not limited to, gancyclovir, etanercept, tacrolimus, sirolimus, voclosporin, cyclosporine, rapamycin, cyclophosphamide, azathioprine, mycophenolate mofetil, methotrexate, glucocorticoid and its analogs, xanthines, stem cell growth factors, lymphotoxins, hematopoietic factors, tumor necrosis factor (TNF) (e.g., TNFα), interleukins (e.g., interleukin-1 (IL-1), IL-2, IL-3, IL-6, IL-10, IL-12, IL-18, and IL-21), colony stimulating factors (e.g., granulocyte-colony stimulating factor (G-CSF) and granulocyte macrophage-colony stimulating factor (GM-CSF)), interferons (e.g., interferons-alpha, interferon-beta, interferon-gamma), the stem cell growth factor designated “S1 factor,” erythropoietin and thrombopoietin, or a combination thereof.

In some embodiments, the payload comprises an immunotoxin. Immunotoxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

In some instances, the payload comprises a nucleic acid polymer. In such instances, the nucleic acid polymer comprises short interfering nucleic acid (siNA), short interfering RNA (siRNA), double-stranded RNA (dsRNA), micro-RNA (miRNA), short hairpin RNA (shRNA), an antisense oligonucleotide. In other instances, the nucleic acid polymer comprises an mRNA, encoding, e.g., a cytotoxic protein or peptide or an apoptotic triggering protein or peptide. Exemplary cytotoxic proteins or peptides include a bacterial cytotoxin such as an alpha-pore forming toxin (e.g., cytolysin A from E. coli), a beta-pore-forming toxin (e.g., α-Hemolysin, PVL-panton Valentine leukocidin, aerolysin, clostridial Epsilon-toxin, Clostridium perfringens enterotoxin), binary toxins (anthrax toxin, edema toxin, C. botulinum C2 toxin, C spirofome toxin, C. perfringens iota toxin, C. difficile cyto-lethal toxins (A and B)), prion, parasporin, a cholesterol-dependent cytolysins (e.g., pneumolysin), a small pore-forming toxin (e.g., Gramicidin A), a cyanotoxin (e.g., microcystins, nodularins), a hemotoxin, a neurotoxin (e.g., botulinum neurotoxin), a cytotoxin, cholera toxin, diphtheria toxin, Pseudomonas exotoxin A, tetanus toxin, or an immunotoxin (idarubicin, ricin A, CRM9, Pokeweed antiviral protein, DT). Exemplary apoptotic triggering proteins or peptides include apoptotic protease activating factor-1 (Apaf-1), cytochrome-c, caspase initiator proteins (CASP2, CASP8, CASP9, CASP10), apoptosis inducing factor (AIF), p53, p73, p63, Bcl-2, Bax, granzyme B, poly-ADP ribose polymerase (PARP), and P 21-activated kinase 2 (PAK2). In additional instances, the nucleic acid polymer comprises a nucleic acid decoy. In some instances, the nucleic acid decoy is a mimic of protein-binding nucleic acids such as RNA-based protein-binding mimics. Exemplary nucleic acid decoys include transactivating region (TAR) decoy and Rev response element (RRE) decoy.

In some cases, the payload is an aptamer. Aptamers are small oligonucleotide or peptide molecules that bind to specific target molecules. Exemplary nucleic acid aptamers include DNA aptamers, RNA aptamers, or XNA aptamers which are RNA and/or DNA aptamers comprising one or more unnatural nucleotides. Exemplary nucleic acid aptamers include ARC19499 (Archemix Corp.), REG1 (Regado Biosciences), and ARC1905 (Ophthotech).

Nucleic acids in accordance with the embodiments described herein optionally include naturally occurring nucleic acids, or one or more nucleotide analogs or have a structure that otherwise differs from that of a naturally occurring nucleic acid. For example, 2′-modifications include halo, alkoxy, and allyloxy groups. In some embodiments, the 2′-OH group is replaced by a group selected from H, OR, R, halo, SH, SR, NH₂, NHR, NR₂ or CN, wherein R is C₁-C₆ alkyl, alkenyl, or alkynyl, and halo is F, Cl, Br, or I. Examples of modified linkages include phosphorothioate and 5′-N-phosphoramidite linkages.

Nucleic acids having a variety of different nucleotide analogs, modified backbones, or non-naturally occurring internucleoside linkages are utilized in accordance with the embodiments described herein. In some cases, nucleic acids include natural nucleosides (i.e., adenosine, thymidine, guanosine, cytidine, uridine, deoxyadenosine, deoxythymidine, deoxyguanosine, and deoxycytidine) or modified nucleosides. Examples of modified nucleotides include base modified nucleoside (e.g., aracytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitorpyrrole, 4-methylindole, 4-thiouridine, 4-thiothymidine, 2-aminoadenosine, 2-thiothymidine, 2-thiouridine, 5-bromocytidine, 5-iodouridine, inosine, 6-azauridine, 6-chloropurine, 7-deazaadenosine, 7-deazaguanosine, 8-azaadenosine, 8-azidoadenosine, benzimidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, 0(6)-methylguanine, and 2-thiocytidine), chemically or biologically modified bases (e.g., methylated bases), modified sugars (e.g., 2′-fluororibose, 2′-aminoribose, 2′-azidoribose, 2′-O-methylribose, L-enantiomeric nucleosides arabinose, and hexose), modified phosphate groups (e.g., phosphorothioates and 5′-N-phosphoramidite linkages), and combinations thereof. Natural and modified nucleotide monomers for the chemical synthesis of nucleic acids are readily available. In some cases, nucleic acids comprising such modifications display enhanced properties relative to nucleic acids consisting only of naturally occurring nucleotides. In some embodiments, nucleic acid modifications described herein are utilized to reduce and/or prevent digestion by nucleases (e.g. exonucleases, endonucleases, etc.). For example, the structure of a nucleic acid may be stabilized by including nucleotide analogs at the 3′ end of one or both strands order to reduce digestion.

Different nucleotide modifications and/or backbone structures may exist at various positions in the nucleic acid. Such modifications include morpholinos, peptide nucleic acids (PNAs), methylphosphonate nucleotides, thiolphosphonate nucleotides, 2′-fluoro N3-P5′-phosphoramidites, 1′,5′-anhydrohexitol nucleic acids (HNAs), or a combination thereof.

Any of the anti-Gal3 antibodies disclosed herein may be conjugated to one or more (e.g. 1, 2, 3, 4, 5, 6, 7, 8, 9, or 10, or more) payloads described herein.

### Conjugation Chemistry

In some instances, the payload is conjugated to an anti-Gal3 antibody described herein by a native ligation. In some instances, the conjugation is as described in: Dawson, et al. “Synthesis of proteins by native chemical ligation,” Science 1994, 266, 776-779; Dawson, et al. “Modulation of Reactivity in Native Chemical Ligation through the Use of Thiol Additives,” J. Am. Chem. Soc. 1997, 119, 4325-4329; Hackeng, et al. “Protein synthesis by native chemical ligation: Expanded scope by using straightforward methodology.,” Proc. Natl. Acad. Sci. USA 1999, 96, 10068-10073; or Wu, et al. “Building complex glycopeptides: Development of a cysteine-free native chemical ligation protocol,” Angew. Chem. Int. Ed. 2006, 45, 4116-4125. In some instances, the conjugation is as described in U.S. Pat. No. 8,936,910.

In some instances, the payload is conjugated to an anti-Gal3 antibody described herein by a site-directed method utilizing a “traceless” coupling technology (Philochem). In some instances, the “traceless” coupling technology utilizes an N-terminal 1,2-aminothiol group on the binding moiety which is then conjugated with a polynucleic acid molecule containing an aldehyde group. (see Casi et al., “Site-specific traceless coupling of potent cytotoxic drugs to recombinant antibodies for pharmacodelivery,” JACS 134(13): 5887-5892 (2012))

In some instances, the payload is conjugated to an anti-Gal3 antibody described herein by a site-directed method utilizing an unnatural amino acid incorporated into the binding moiety. In some instances, the unnatural amino acid comprises p-acetylphenylalanine (pAcPhe). In some instances, the keto group of pAcPhe is selectively coupled to an alkoxy-amine derivatived conjugating moiety to form an oxime bond. (see Axup et al., “Synthesis of site-specific antibody-drug conjugates using unnatural amino acids,” PNAS 109(40): 16101-16106 (2012)).

In some instances, the payload is conjugated to an anti-Gal3 antibody described herein by a site-directed method utilizing an enzyme-catalyzed process. In some instances, the site-directed method utilizes SMARTag™ technology (Redwood). In some instances, the SMARTag™ technology comprises generation of a formylglycine (FGly) residue from cysteine by formylglycine-generating enzyme (FGE) through an oxidation process under the presence of an aldehyde tag and the subsequent conjugation of FGly to an alkylhydraine-functionalized polynucleic acid molecule via hydrazino-Pictet-Spengler (HIPS) ligation. (see Wu et al., “Site-specific chemical modification of recombinant proteins produced in mammalian cells by using the genetically encoded aldehyde tag,” PNAS 106(9): 3000-3005 (2009); Agarwal, et al., “A Pictet-Spengler ligation for protein chemical modification,” PNAS 110(1): 46-51 (2013)).

In some instances, the enzyme-catalyzed process comprises microbial transglutaminase (mTG). In some cases, the payload is conjugated to the anti-Gal3 antibody utilizing a microbial transglutaminase catalyzed process. In some instances, mTG catalyzes the formation of a covalent bond between the amide side chain of a glutamine within the recognition sequence and a primary amine of a functionalized polynucleic acid molecule. In some instances, mTG is produced from Streptomyces mobarensis. (see Strop et al., “Location matters: site of conjugation modulates stability and pharmacokinetics of antibody drug conjugates,” Chemistry and Biology 20(2) 161-167 (2013)).

In some instances, the payload is conjugated to an anti-Gal3 antibody by a method as described in PCT Publication No. WO2014/140317, which utilizes a sequence-specific transpeptidase and is hereby expressly incorporated by reference in its entirety.

In some instances, the payload is conjugated to an anti-Gal3 antibody described herein by a method as described in U.S. Patent Publication Nos. 2015/0105539 and 2015/0105540.

### Linker

In some instances, a linker described herein comprises a natural or synthetic polymer, consisting of long chains of branched or unbranched monomers, and/or cross-linked network of monomers in two or three dimensions. In some instances, the linker includes a polysaccharide, lignin, rubber, or polyalkylene oxide (e.g., polyethylene glycol).

In some instances, the linker includes, but is not limited to, alpha-, omega-dihydroxylpolyethyleneglycol, biodegradable lactone-based polymer, e.g. polyacrylic acid, polylactide acid (PLA), poly(glycolic acid) (PGA), polypropylene, polystyrene, polyolefin, polyamide, polycyanoacrylate, polyimide, polyethyleneterephthalate (PET, PETG), polyethylene terephthalate (PETE), polytetramethylene glycol (PTG), or polyurethane as well as mixtures thereof. As used herein, a mixture refers to the use of different polymers within the same compound as well as in reference to block copolymers. In some cases, block copolymers are polymers wherein at least one section of a polymer is built up from monomers of another polymer. In some instances, the linker comprises polyalkylene oxide. In some instances, the linker comprises PEG. In some instances, the linker comprises polyethylene imide (PEI) or hydroxy ethyl starch (HES).

In some cases, the polyalkylene oxide (e.g., PEG) is a polydisperse or monodisperse compound. In some instances, polydisperse material comprises disperse distribution of different molecular weight of the material, characterized by mean weight (weight average) size and dispersity. In some instances, the monodisperse PEG comprises one size of molecules. In some embodiments, the linker is poly- or monodispersed polyalkylene oxide (e.g., PEG) and the indicated molecular weight represents an average of the molecular weight of the polyalkylene oxide, e.g., PEG, molecules.

In some embodiments, the linker comprises a polyalkylene oxide (e.g., PEG) and the molecular weight of the polyalkylene oxide (e.g., PEG) is about 200, 300, 400, 500, 600, 700, 800, 900, 1000, 1100, 1200, 1300, 1400, 1450, 1500, 1600, 1700, 1800, 1900, 2000, 2100, 2200, 2300, 2400, 2500, 2600, 2700, 2800, 2900, 3000, 3250, 3350, 3500, 3750, 4000, 4250, 4500, 4600, 4750, 5000, 5500, 6000, 6500, 7000, 7500, 8000, 10,000, 12,000, 20,000, 35,000, 40,000, 50,000, 60,000, or 100,000 Da.

In some embodiments, the polyalkylene oxide (e.g., PEG) is a discrete PEG, in which the discrete PEG is a polymeric PEG comprising more than one repeating ethylene oxide unit. In some instances, a discrete PEG (dPEG) comprises from 2 to 60, from 2 to 50, or from 2 to 48 repeating ethylene oxide units. In some instances, a dPEG comprises about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units. In some instances, a dPEG comprises about 2 or more repeating ethylene oxide units. In some cases, a dPEG is synthesized as a single molecular weight compound from pure (e.g., about 95%, 98%, 99%, or 99.5%) starting material in a step-wise fashion. In some cases, a dPEG has a specific molecular weight, rather than an average molecular weight.

In some instances, the linker is a discrete PEG, optionally comprising from 2 to 60, from 2 to 50, or from 2 to 48 repeating ethylene oxide units. In some cases, the linker comprises a dPEG comprising about 2, 3, 4, 5, 6, 7, 8, 9, 10, 11, 12, 13, 14, 15, 16, 17, 18, 19, 20, 22, 24, 26, 28, 30, 35, 40, 42, 48, 50 or more repeating ethylene oxide units.

In some embodiments, the linker is a polypeptide linker. In some instances, the polypeptide linker comprises at least 2, 3, 4, 5, 6, 7, 8, 10, 15, 20, 25, 30, 35, 40, 45, 50, 60, 70, 80, 90, 100, or more amino acid residues. In some instances, the polypeptide linker comprises at least 2, 3, 4, 5, 6, 7, 8, or more amino acid residues. In some instances, the polypeptide linker comprises at most 2, 3, 4, 5, 6, 7, 8, or less amino acid residues. In some cases, the polypeptide linker is a cleavable polypeptide linker (e.g., either enzymatically or chemically). In some cases, the polypeptide linker is a non-cleavable polypeptide linker. In some instances, the polypeptide linker comprises Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some instances, the polypeptide linker comprises a peptide such as: Val-Cit (valine-citrulline), Gly-Gly-Phe-Gly, Phe-Lys, Val-Lys, Gly-Phe-Lys, Phe-Phe-Lys, Ala-Lys, Val-Arg, Phe-Cit, Phe-Arg, Leu-Cit, Ile-Cit, Trp-Cit, Phe-Ala, Ala-Leu-Ala-Leu, or Gly-Phe-Leu-Gly. In some cases, the polypeptide linker comprises L-amino acids, D-amino acids, or a mixture of both L- and D-amino acids.

In some instances, the linker comprises a homobifunctional linker. Exemplary homobifunctional linkers include, but are not limited to, Lomant's reagent dithiobis (succinimidylpropionate) DSP, 3′3′-dithiobis(sulfosuccinimidyl proprionate (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[D-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide).

In some embodiments, the linker comprises a heterobifunctional linker. Exemplary heterobifunctional linker include, but are not limited to, amine-reactive and sulfhydryl cross-linkers such as N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(ρ-maleimidophenyl)butyrate (sMPB), sulfosuccinimidyl-4-(ρ-maleimidophenyl)butyrate (sulfo-sMPB), N-(γ-maleimidobutyryloxy)succinimide ester (GMBs), N-(γ-maleimidobutyryloxy)sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (sIAXX), succinimidyl 4-(((iodoacetyl)amino)methyl)cyclohexane-1-carboxylate (sIAC), succinimidyl 6-((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M₂C2H), 3-(2-pyridyldithio)propionyl hydrazide (PDPH), amine-reactive and photoreactive cross-linkers such as N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(ρ-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenylamino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), ρ-nitrophenyl diazopyruvate (pNPDP), ρ-nitrophenyl-2-diazo-3,3,3-trifluoropropionate (PNP-DTP), sulfhydryl-reactive and photoreactive cross-linkers such as1-(ρ-Azidosalicylamido)-4-(iodoacetamido)butane (AsIB), N-[4-(ρ-azidosalicylamido)butyl]-3′-(2′-pyridyldithio)propionamide (APDP), benzophenone-4-iodoacetamide, benzophenone-4-maleimide carbonyl-reactive and photoreactive cross-linkers such as ρ-azidobenzoyl hydrazide (ABH), carboxylate-reactive and photoreactive cross-linkers such as 4-(ρ-azidosalicylamido)butylamine (AsBA), and arginine-reactive and photoreactive cross-linkers such as p-azidophenyl glyoxal (APG).

In some embodiments, the linker comprises a benzoic acid group, or its derivatives thereof. In some instances, the benzoic acid group or its derivatives thereof comprise paraaminobenzoic acid (PABA). In some instances, the benzoic acid group or its derivatives thereof comprise gamma-aminobutyric acid (GABA).

In some embodiments, the linker comprises one or more of a maleimide group, a peptide moiety, and/or a benzoic acid group, in any combination. In some embodiments, the linker comprises a combination of a maleimide group, a peptide moiety, and/or a benzoic acid group. In some instances, the maleimide group is maleimidocaproyl (mc). In some instances, the peptide group is val-cit. In some instances, the benzoic acid group is PABA. In some instances, the linker comprises a mc-val-cit group. In some cases, the linker comprises a val-cit-PABA group. In additional cases, the linker comprises a mc-val-cit-PABA group.

In some embodiments, the linker is a self-immolative linker or a self-elimination linker. In some cases, the linker is a self-immolative linker. In other cases, the linker is a self-elimination linker (e.g., a cyclization self-elimination linker). In some instances, the linker comprises a linker described in U.S. Pat. No. 9,089,614 or PCT Publication No. WO2015038426.

In some embodiments, the linker is a dendritic type linker. In some instances, the dendritic type linker comprises a branching, multifunctional linker moiety. In some instances, the dendritic type linker comprises PAMAM dendrimers.

In some embodiments, the linker is a traceless linker or a linker in which after cleavage does not leave behind a linker moiety (e.g., an atom or a linker group) to the antibody or payload. Exemplary traceless linkers include, but are not limited to, germanium linkers, silicium linkers, sulfur linkers, selenium linkers, nitrogen linkers, phosphorus linkers, boron linkers, chromium linkers, or phenylhydrazide linker. In some cases, the linker is a traceless aryl-triazene linker as described in Hejesen, et al., “A traceless aryl-triazene linker for DNA-directed chemistry,” Org Biomol Chem 11(15): 2493-2497 (2013). In some instances, the linker is a traceless linker described in Blaney, et al., “Traceless solid-phase organic synthesis,” Chem. Rev. 102: 2607-2024 (2002). In some instances, a linker is a traceless linker as described in U.S. Pat. No. 6,821,783.

### Pharmaceutical Compositions

In some embodiments, an anti-Gal3 antibody or binding fragment thereof disclosed herein is further formulated as a pharmaceutical composition.

As applied to any of the anti-Gal3 antibodies or binding fragments thereof formulated as a pharmaceutical composition, in some embodiments, the anti-Gal3 antibody or binding fragment thereof comprises any one or more sequences (such as a V_{H}-CDR1, V_{H}-CDR2, V_{H}-CDR3, V_{L}-CDR1, V_{L}-CDR2, V_{L}-CDR3, heavy chain variable region, light chain variable region, heavy chain, or light chain sequence) provided throughout this disclosure. In some embodiments, the anti-Gal3 antibody or binding fragment thereof formulated as a pharmaceutical composition comprises any one or more sequences as shown in FIG. 18-32, including any one or more CDRs, heavy chain variable regions, light chain variable regions, heavy chains, light chains, combinations of CDRs, combinations of variable regions, or combinations of heavy chain and light chain described therein. In some embodiments, the anti-Gal3 antibody or binding fragment thereof formulated as a pharmaceutical composition comprises a peptide sequence having at least 80%, 85%, 90%, 95%, 99%, or 100% homology to the peptide sequence encoded by any one or more of the nucleic acid sequences as shown in FIG. 37-40, including any nucleic sequences encoding for a heavy chain variable region, light chain variable region, heavy chain, or light chain.

In some embodiments, the anti-Gal3 antibody or binding fragment thereof formulated as a pharmaceutical composition comprises (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3. In some embodiments, the V_{H}-CDR1 comprises an amino acid sequence selected from SEQ ID NOs: 27-44, 245-246, 397-399, 588-615, the V_{H}-CDR2 comprises an amino acid sequence selected from SEQ ID NOs: 45-60, 247-248, 400-406, 616-643, the V_{H}-CDR3 comprises an amino acid sequence selected from SEQ ID NOs: 61-81, 249-250, 407-416, 644-671, the V_{L}-CDR1 comprises an amino acid sequence selected from SEQ ID NOs: 82-101, 251-252, 417-426, 672-699, the V_{L}-CDR2 comprises an amino acid sequence selected from SEQ ID NOs: 102-116, 253, 427-428, 700-727, and the V_{L}-CDR3 comprises an amino acid sequence selected from SEQ ID NOs: 117-135, 254-255, 429-434, 728-755.

In some embodiments, exemplary V_{H}-CDR1 sequences for anti-Gal3 antibodies or binding fragment thereof formulated as pharmaceutical compositions are depicted in FIG. 18. In some embodiments, exemplary V_{H}-CDR2 sequences for anti-Gal3 antibodies or binding fragment thereof formulated as pharmaceutical compositions are depicted in FIG. 19. In some embodiments, exemplary V_{H}-CDR3 sequences for anti-Gal3 antibodies or binding fragment thereof formulated as pharmaceutical compositions are depicted in FIG. 20. In some embodiments, exemplary V_{L}-CDR1 sequences for anti-Gal3 antibodies or binding fragment thereof formulated as pharmaceutical compositions are depicted in FIG. 21. In some embodiments, exemplary V_{L}-CDR2 sequences for anti-Gal3 antibodies or binding fragment thereof formulated as pharmaceutical compositions are depicted in FIG. 22. In some embodiments, exemplary V_{L}-CDR3 sequences are depicted in FIG. 23.

In some embodiments, the V_{H} for anti-Gal3 antibodies or binding fragment thereof formulated as pharmaceutical compositions comprises an amino acid sequence having at least 75%, 80%, 85%, 90%, 95%, or 100% sequence identity to any sequence according to SEQ ID NOs: 147-160. In some embodiments, the V_{H} for anti-Gal3 antibodies or binding fragment thereof formulated as pharmaceutical compositions is selected from the group consisting of SEQ ID NOs: 147-160. In some embodiments, exemplary V_{H} for anti-Gal3 antibodies or binding fragment thereof formulated as pharmaceutical compositions are depicted in FIG. 24.

In some embodiments, the V_{L} for anti-Gal3 antibodies or binding fragment thereof formulated as pharmaceutical compositions comprises an amino acid sequence having at least 75%, 80%, 85%, 90%, 95%, or 100% sequence identity to any sequence according to SEQ ID NOs: 173-187. In some embodiments, the V_{L} is selected from the group consisting of SEQ ID NOs: 173-187. In some embodiments, exemplary V_{L} for anti-Gal3 antibodies or binding fragment thereof formulated as pharmaceutical compositions are depicted in FIG. 25.

In some embodiments, the anti-Gal3 antibody or binding fragment thereof formulated as a pharmaceutical composition comprises a) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173; b) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174; c) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175; d) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176; e) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177; f) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178; g) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179; h) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180; i) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181; j) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182; k) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183; 1) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184; m) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185; n) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186; or o) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187. In some embodiments, exemplary combinations of heavy chain variable region CDRs for anti-Gal3 antibodies or binding fragment thereof formulated as pharmaceutical compositions are depicted in FIG. 28. In some embodiments, exemplary combinations of light chain variable region CDRs for anti-Gal3 antibodies or binding fragment thereof formulated as pharmaceutical compositions are depicted in FIG. 29.

In some embodiments, the anti-Gal3 antibody or binding fragment thereof formulated as a pharmaceutical composition comprises a) the heavy chain variable region of SEQ ID NO: 147 and the light chain variable region of SEQ ID NO: 173; b) the heavy chain variable region of SEQ ID NO: 148 and the light chain variable region of SEQ ID NO: 174; c) the heavy chain variable region of SEQ ID NO: 149 and the light chain variable region of SEQ ID NO: 175; d) the heavy chain variable region of SEQ ID NO: 150 and the light chain variable region of SEQ ID NO: 176; e) the heavy chain variable region of SEQ ID NO: 151 and the light chain variable region of SEQ ID NO: 177; f) the heavy chain variable region of SEQ ID NO: 152 and the light chain variable region of SEQ ID NO: 178; g) the heavy chain variable region of SEQ ID NO: 153 and the light chain variable region of SEQ ID NO: 179; h) the heavy chain variable region of SEQ ID NO: 154 and the light chain variable region of SEQ ID NO: 180; i) the heavy chain variable region of SEQ ID NO: 155 and the light chain variable region of SEQ ID NO: 181; j) the heavy chain variable region of SEQ ID NO: 156 and the light chain variable region of SEQ ID NO: 182; k) the heavy chain variable region of SEQ ID NO: 157 and the light chain variable region of SEQ ID NO: 183; 1) the heavy chain variable region of SEQ ID NO: 155 and the light chain variable region of SEQ ID NO: 184; m) the heavy chain variable region of SEQ ID NO: 158 and the light chain variable region of SEQ ID NO: 185; n) the heavy chain variable region of SEQ ID NO: 159 and the light chain variable region of SEQ ID NO: 186; or o) the heavy chain variable region of SEQ ID NO: 160 and the light chain variable region of SEQ ID NO: 187.

In some embodiments, the anti-Gal3 antibody or binding fragment thereof formulated as a pharmaceutical composition comprises the heavy chain (HC) sequence of any one of SEQ ID NOs: 201-216. In some embodiments, exemplary HC sequences for anti-Gal3 antibodies or binding fragment thereof formulated as pharmaceutical compositions are depicted in FIG. 26.

In some embodiments, the anti-Gal3 antibody or binding fragment thereof formulated as a pharmaceutical composition comprises the light chain (LC) sequence of any one of SEQ ID NOs: 229-243. In some embodiments, exemplary LC sequences for anti-Gal3 antibodies or binding fragment thereof formulated as pharmaceutical compositions are depicted in FIG. 27.

In some embodiments, the anti-Gal3 antibody or binding fragment thereof formulated as a pharmaceutical composition is selected from the group consisting of the anti-Gal3 antibody or binding fragment of at least one of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof. In some embodiments, the anti-Gal3 antibody or binding fragment thereof formulated as a pharmaceutical composition is selected from the group consisting of F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, and 846.4D5, or a binding fragment thereof. In some embodiments, the anti-Gal3 antibody or binding fragment thereof disrupts an interaction between Gal3 and an antibody selected from 846.4D5, 15F10.2D6, F846C.1B2, and F846C.1H12. In some embodiments, the heavy and light chain CDRs associated with each of the foregoing antibodies used in pharmaceutical compositions are depicted in FIG. 30. In some embodiments, the V_{H} and V_{L} associated with each of the foregoing antibodies used in pharmaceutical compositions are depicted in FIG. 31. In some embodiments, the HC and LC associated with each of the foregoing antibodies used in pharmaceutical compositions are depicted in FIG. 32.

In some embodiments, the anti-Gal3 antibody or binding fragment thereof formulated for a pharmaceutical composition binds to one or more peptides of SEQ ID NOs: 3-26.

In some instances, the pharmaceutical composition is formulated for administration to a subject by one or more administration routes, including but not limited to, parenteral (e.g., intravenous, subcutaneous, intramuscular, intraarterial, intradermal, intraperitoneal, intravitreal, intracerebral, or intracerebroventricular), oral, intranasal, buccal, rectal, or transdermal administration routes. In some instances, the pharmaceutical composition described herein is formulated for parenteral (e.g., intravenous, subcutaneous, intramuscular, intraarterial, intradermal, intraperitoneal, intravitreal, intracerebral, or intracerebroventricular) administration. In other instances, the pharmaceutical composition described herein is formulated for oral administration. In still other instances, the pharmaceutical composition describe herein is formulated for intranasal administration. Proper formulation is dependent upon the route of administration chosen. Techniques for formulation and administration of the compounds described herein are known to those skilled in the art.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or carrier can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “adjuvant” as used herein refers to a substance, compound, or material that stimulates the immune response and increases the efficacy of protective immunity and is administered in conjunction with an immunogenic antigen, epitope, or composition. Adjuvants serve to enhance immune responses by enabling a continual release of antigen, up-regulation of cytokines and chemokines, cellular recruitment at the site of administration, increased antigen uptake and presentation in antigen presenting cells, or activation of antigen presenting cells and inflammasomes. Commonly used adjuvants include but are not limited to alum, aluminum salts, aluminum sulfate, aluminum hydroxide, aluminum phosphate, calcium phosphate hydroxide, potassium aluminum sulfate, oils, mineral oil, paraffin oil, oil-in-water emulsions, detergents, MF59®, squalene, AS03, α-tocopherol, polysorbate 80, AS04, monophosphoryl lipid A, virosomes, nucleic acids, polyinosinic:polycytidylic acid, saponins, QS-21, proteins, flagellin, cytokines, chemokines, IL-1, IL-2, IL-12, IL-15, IL-21, imidazoquinolines, CpG oligonucleotides, lipids, phospholipids, dioleoyl phosphatidylcholine (DOPC), trehalose dimycolate, peptidoglycans, bacterial extracts, lipopolysaccharides, or Freund's Adjuvant, or any combination thereof.

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multi-particulate formulations (e.g., nanoparticle formulations), and mixed immediate and controlled release formulations.

In some instances, the pharmaceutical compositions further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-(hydroxymethyl)aminomethane; and buffers such as citrate/dextrose, sodium bicarbonate and ammonium chloride. Such acids, bases and buffers are included in an amount required to maintain pH of the composition in an acceptable range.

In some instances, the pharmaceutical compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical compositions further include diluents which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel©; dibasic calcium phosphate, dicalcium phosphate dihydrate; tricalcium phosphate, calcium phosphate; anhydrous lactose, spray-dried lactose; pregelatinized starch, compressible sugar, such as Di-Pac© (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

### Therapeutic Regimens

In some embodiments, the anti-Gal3 antibodies, binding fragments, or antigen binding molecules disclosed herein are administered for therapeutic applications. In some embodiments, the anti-Gal3 antibody, binding fragment, or antigen binding molecule is administered once per day, twice per day, three times per day or more. The anti-Gal3 antibody, binding fragment, or antigen binding molecule is administered daily, every day, every alternate day, five days a week, once a week, every other week, two weeks per month, three weeks per month, once a month, twice a month, three times per month, or more. The anti-Gal3 antibody, binding fragment, or antigen binding molecule is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the anti-Gal3 antibody, binding fragment, or antigen binding molecule is given continuously; alternatively, the dose of the anti-Gal3 antibody, binding fragment, or antigen binding molecule being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's condition has occurred, a maintenance dose is administered if necessary. Subsequently, the dosage or the frequency of administration, or both, can be reduced, as a function of the symptoms, to a level at which the treated disease, disorder, or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in humans. The dosage of such compounds lies preferably within a range of circulating concentrations that include the ED50 with minimal toxicity. The dosage varies within this range depending upon the dosage form employed and the route of administration utilized.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include an anti-Gal3 antibody as disclosed herein, host cells for producing one or more antibodies described herein, and/or vectors comprising nucleic acid molecules that encode the antibodies described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

The invention(s) is/are generally disclosed herein using affirmative language to describe the numerous embodiments. The invention also includes embodiments in which subject matter is excluded, in full or in part, such as substances or materials, method steps and conditions, protocols, or procedures.

Some embodiments provided herein are described by way of the following provided numbered arrangements and also provided as possible combinations or overlapping embodiments:

1. An anti-Gal3 antibody or binding fragment thereof comprising (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3; wherein

the V_{H}-CDR1 comprises an amino acid sequence selected from SEQ ID NOs: 36-44, 588-615,

the V_{H}-CDR2 comprises an amino acid sequence selected from SEQ ID NOs: 54-60, 616-643,

the V_{H}-CDR3 comprises an amino acid sequence selected from SEQ ID NOs: 70-81, 644-671,

the V_{L}-CDR1 comprises an amino acid sequence selected from SEQ ID NOs: 92-101, 672-699,

the V_{L}-CDR2 comprises an amino acid sequence selected from SEQ ID NOs: 111-116, 700-727, and

the V_{L}-CDR3 comprises an amino acid sequence selected from SEQ ID NOs: 127-135, 728-755.

2. The anti-Gal3 antibody or binding fragment thereof of arrangement 1, wherein the heavy chain variable region comprises an amino acid sequence having at least 75%, 80%, 85%, 90%, 95%, or 100% sequence identity to any sequence according to SEQ ID NOs: 147-160, 756-783.

3. The anti-Gal3 antibody or binding fragment thereof of arrangement 1 or 2, wherein the heavy chain variable region is selected from the group consisting of SEQ ID NOs: 147-160, 756-783.

4. The anti-Gal3 antibody or binding fragment thereof of any one of arrangements 1-3, wherein the light chain variable region comprises an amino acid sequence having at least 75%, 80%, 85%, 90%, 95%, or 100% sequence identity to any sequence according to SEQ ID NOs: 173-187, 784-811.

5. The anti-Gal3 antibody or binding fragment thereof of any one of arrangements 1-4, wherein the light chain variable region is selected from the group consisting of SEQ ID NOs: 173-187, 784-811.

6. The anti-Gal3 antibody or binding fragment thereof of any one of arrangements 1-5, comprising:

1) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173;

2) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

3) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175;

4) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176;

5) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177;

6) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178;

7) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179;

8) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180;

9) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181;

10) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182;

11) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183;

12) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184;

13) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185;

14) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186;

15) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187;

16) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784;

17) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785;

18) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786;

19) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787;

20) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788;

21) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789;

22) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790;

23) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791;

24) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792;

25) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793;

26) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794;

27) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795;

28) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796;

29) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797;

30) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798;

31) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799;

32) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800;

33) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801;

34) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802;

35) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803;

36) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804;

37) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805;

38) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806;

39) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807;

40) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808;

41) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809;

42) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or 43) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

7. The anti-Gal3 antibody or binding fragment thereof of any one of arrangements 1-6, comprising:

1) the heavy chain variable region of SEQ ID NO: 147 and the light chain variable region of SEQ ID NO: 173;

2) the heavy chain variable region of SEQ ID NO: 148 and the light chain variable region of SEQ ID NO: 174;

3) the heavy chain variable region of SEQ ID NO: 149 and the light chain variable region of SEQ ID NO: 175;

4) the heavy chain variable region of SEQ ID NO: 150 and the light chain variable region of SEQ ID NO: 176;

5) the heavy chain variable region of SEQ ID NO: 151 and the light chain variable region of SEQ ID NO: 177;

6) the heavy chain variable region of SEQ ID NO: 152 and the light chain variable region of SEQ ID NO: 178;

7) the heavy chain variable region of SEQ ID NO: 153 and the light chain variable region of SEQ ID NO: 179;

8) the heavy chain variable region of SEQ ID NO: 154 and the light chain variable region of SEQ ID NO: 180;

9) the heavy chain variable region of SEQ ID NO: 155 and the light chain variable region of SEQ ID NO: 181;

10) the heavy chain variable region of SEQ ID NO: 156 and the light chain variable region of SEQ ID NO: 182;

11) the heavy chain variable region of SEQ ID NO: 157 and the light chain variable region of SEQ ID NO: 183;

12) the heavy chain variable region of SEQ ID NO: 155 and the light chain variable region of SEQ ID NO: 184;

13) the heavy chain variable region of SEQ ID NO: 158 and the light chain variable region of SEQ ID NO: 185;

14) the heavy chain variable region of SEQ ID NO: 159 and the light chain variable region of SEQ ID NO: 186;

15) the heavy chain variable region of SEQ ID NO: 160 and the light chain variable region of SEQ ID NO: 187;

16) the heavy chain variable region of SEQ ID NO: 756 and the light chain variable region of SEQ ID NO: 784;

17) the heavy chain variable region of SEQ ID NO: 757 and the light chain variable region of SEQ ID NO: 785;

18) the heavy chain variable region of SEQ ID NO: 758 and the light chain variable region of SEQ ID NO: 786;

19) the heavy chain variable region of SEQ ID NO: 759 and the light chain variable region of SEQ ID NO: 787;

20) the heavy chain variable region of SEQ ID NO: 760 and the light chain variable region of SEQ ID NO: 788;

21) the heavy chain variable region of SEQ ID NO: 761 and the light chain variable region of SEQ ID NO: 789;

22) the heavy chain variable region of SEQ ID NO: 762 and the light chain variable region of SEQ ID NO: 790;

23) the heavy chain variable region of SEQ ID NO: 763 and the light chain variable region of SEQ ID NO: 791;

24) the heavy chain variable region of SEQ ID NO: 764 and the light chain variable region of SEQ ID NO: 792;

25) the heavy chain variable region of SEQ ID NO: 765 and the light chain variable region of SEQ ID NO: 793;

26) the heavy chain variable region of SEQ ID NO: 766 and the light chain variable region of SEQ ID NO: 794;

27) the heavy chain variable region of SEQ ID NO: 767 and the light chain variable region of SEQ ID NO: 795;

28) the heavy chain variable region of SEQ ID NO: 768 and the light chain variable region of SEQ ID NO: 796;

29) the heavy chain variable region of SEQ ID NO: 769 and the light chain variable region of SEQ ID NO: 797;

30) the heavy chain variable region of SEQ ID NO: 770 and the light chain variable region of SEQ ID NO: 798;

31) the heavy chain variable region of SEQ ID NO: 771 and the light chain variable region of SEQ ID NO: 799;

32) the heavy chain variable region of SEQ ID NO: 772 and the light chain variable region of SEQ ID NO: 800;

33) the heavy chain variable region of SEQ ID NO: 773 and the light chain variable region of SEQ ID NO: 801;

34) the heavy chain variable region of SEQ ID NO: 774 and the light chain variable region of SEQ ID NO: 802;

35) the heavy chain variable region of SEQ ID NO: 775 and the light chain variable region of SEQ ID NO: 803;

36) the heavy chain variable region of SEQ ID NO: 776 and the light chain variable region of SEQ ID NO: 804;

37) the heavy chain variable region of SEQ ID NO: 777 and the light chain variable region of SEQ ID NO: 805;

38) the heavy chain variable region of SEQ ID NO: 778 and the light chain variable region of SEQ ID NO: 806;

39) the heavy chain variable region of SEQ ID NO: 779 and the light chain variable region of SEQ ID NO: 807;

40) the heavy chain variable region of SEQ ID NO: 780 and the light chain variable region of SEQ ID NO: 808;

41) the heavy chain variable region of SEQ ID NO: 781 and the light chain variable region of SEQ ID NO: 809;

42) the heavy chain variable region of SEQ ID NO: 782 and the light chain variable region of SEQ ID NO: 810; or

43) the heavy chain variable region of SEQ ID NO: 783 and the light chain variable region of SEQ ID NO: 811.

8. The anti-Gal3 antibody or binding fragment thereof of any one of arrangements 1-7, wherein the anti-Gal3 antibody or binding fragment thereof is selected from the group consisting of F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

9. The anti-Gal3 antibody or binding fragment thereof of any one of arrangements 1-8, wherein the anti-Gal3 antibody or binding fragment thereof is selected from the group consisting of F846C.1B2, F846C.1F5, F846C.1H12, F846C.2H3, F846TC.14E4, F846TC.16B5, F846TC.7F10, F849C.8D10, 846.4D5, or a binding fragment thereof.

10. The anti-Gal3 antibody or binding fragment thereof of any one of arrangements 1-10, wherein the anti-Gal3 antibody or binding fragment thereof binds to one or more peptides of SEQ ID NOs: 3-26.

11. A method of treating a neurological disorder in a subject in need thereof, comprising:

administering to the subject an effective amount of an anti-Gal3 antibody or binding fragment thereof, thereby treating the neurological order.

12. The method of arrangement 11, further comprising selecting the subject as having the neurological disorder or at risk of contracting the neurological disorder prior to the administering step.

13. The method of arrangement 11 or 12, further comprising detecting an amelioration of symptoms associated with the neurological disorder after the administering step.

14. The method of any one of arrangements 11-13, wherein the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof.

15. The method of any one of arrangements 11-14, wherein the neurological disorder is Alzheimer's disease, and wherein the anti-Gal3 antibody or binding fragment thereof disrupts binding between Gal3 and amyloid precursor protein (APP) or amyloid beta (Aβ), or both.

16. The method of arrangement 15, wherein the APP comprises the sequence of APP695 (SEQ ID NO: 2).

17. The method of arrangement 15 or 16, wherein the Aβ comprises Aβ monomers, Aβ oligomers, Aβ fibrils, or any combination thereof.

18. The method of any one of arrangements 15-17, wherein the Aβ comprises the sequence of Aβ42 (SEQ ID NO: 244).

19. The method of any one of arrangements 15-18, wherein the anti-Gal3 antibody or binding fragment thereof reduces the binding between Gal3 and APP or Aβ, or both, by at least 50%, 55%, 60%, 65%, 70%, 75%, 80%, 85%, 90%, 95%, 96%, 97%, 98%, or 99%, or any percentage within a range defined by any two aforementioned percentages.

20. The method of any one of arrangements 11-19, wherein the anti-Gal3 antibody or binding fragment thereof promotes phagocytic function of microglia in the subject.

21. The method of any one of arrangements 11-20, wherein the anti-Gal3 antibody or binding fragment thereof decreases phospho-Tau levels or Gal3 levels, or both, in the brain of the subject.

22. The method of any one of arrangements 11-21, wherein the anti-Gal3 antibody or binding fragment thereof inhibits AD-mediated activation of microglia in the subject.

23. The method of arrangement 22, wherein the AD-mediated activation of microglia is inhibited by at least 50%, 55%, 60%, 65%, 70%, 75%, 80%, 85%, 90%, 95%, 96%, 97%, 98%, or 99%, or any percentage within a range defined by any two aforementioned percentages.

24. The method of any one of arrangements 11-23, wherein the anti-Gal3 antibody or binding fragment thereof inhibits Aβ fibril or oligomer formation in the subject.

25. The method of arrangement 24, wherein the Aβ fibril or oligomer formation is inhibited by at least 50%, 55%, 60%, 65%, 70%, 75%, 80%, 85%, 90%, 95%, 96%, 97%, 98%, or 99%, or any percentage within a range defined by any two aforementioned percentages.

26. The method of any one of arrangements 11-25, wherein the anti-Gal3 antibody or binding fragment thereof promotes neuronal regeneration in the subject.

27. The method of any one of arrangements 11-26, wherein the anti-Gal3 antibody or binding fragment thereof disrupts binding between Gal3 and Toll-like receptor 4 (TLR4) or triggering receptor expressed on myeloid cells 2 (TREM2), or both.

28. The method of arrangement 27, wherein the binding between Gal3 and TLR4 or TREM2, or both, is disrupted by at least 50%, 55%, 60%, 65%, 70%, 75%, 80%, 85%, 90%, 95%, 96%, 97%, 98%, or 99%, or any percentage within a range defined by any two aforementioned percentages.

29. The method of any one of arrangements 11-28, wherein more than one anti-Gal3 antibody or binding fragment thereof is administered to the subject.

30. The method of any one of arrangements 11-29, wherein the anti-Gal3 antibody or binding fragment thereof is administered with one or more additional therapeutic compositions.

31. The method of arrangement 30, wherein the one or more additional therapeutic compositions comprise a cholinesterase inhibitor, an NMDA receptor antagonist, or both.

32. The method of arrangement 31, wherein the cholinesterase inhibitor comprises tacrine, rivastigmine, galantamine, donepezil, or any combination thereof.

33. The method of arrangement 31 or 32, wherein the NMDA receptor antagonist comprises memantine.

34. A method of disrupting binding between Gal3 and APP or Aβ, or both, comprising contacting the APP or Aβ, or both, with an anti-Gal3 antibody or binding fragment thereof, thereby disrupting the binding between Gal3 and APP.

35. The method of arrangement 34, wherein the APP or Aβ, or both, is soluble or part of a first cell.

36. The method of arrangement 34 or 35, wherein the Gal3 is soluble or part of a second cell.

37. The method of any one of arrangements 34-36, wherein the APP comprises the sequence of APP695 (SEQ ID NO: 2).

38. The method of any one of arrangements 34-37, wherein the Aβ comprises Aβ monomers, Aβ oligomers, Aβ fibrils, or any combination thereof.

39. The method of any one of arrangements 34-38, wherein the Aβ comprises the sequence of Aβ42 (SEQ ID NO: 244).

40. The method of any one of arrangements 34-39, wherein the anti-Gal3 antibody or binding fragment thereof reduces the binding between Gal3 and APP or Aβ, or both, by at least 85%.

41. The method of any one of arrangements 34-40, wherein the anti-Gal3 antibody or binding fragment thereof reduces the binding between Gal3 and APP or Aβ, or both, by at least 90%.

42. The method of any one of arrangements 34-41, wherein the anti-Gal3 antibody or binding fragment thereof reduces the binding between Gal3 and APP or Aβ, or both, by at least 95%.

43. The method of any one of arrangements 34-42, wherein the APP is contacted with more than one anti-Gal3 antibody or binding fragment thereof.

44. A method of treating a proteopathy in a subject in need thereof, comprising:

administering to the subject an effective amount of an anti-Gal3 antibody or binding fragment thereof, thereby treating the proteopathy in the subject.

45. The method of arrangement 44, further comprising selecting the subject as having the proteopathy or at risk of contracting the proteopathy prior to the administering step.

46. The method of arrangement 44 or 45, further comprising detecting an amelioration of symptoms associated with the proteopathy after the administering step.

47. The method of any one of arrangements 44-46, wherein treating the proteopathy comprises treating an active proteopathy, or a prophylactic treatment, or both, in the subject.

48. The method of any one of arrangements 44-47, wherein the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof.

49. The method of any one of arrangements 44-48, wherein more than one anti-Gal3 antibody or binding fragment thereof is administered to the subject.

50. The method of any one of arrangements 44-49, wherein the anti-Gal3 antibody or binding fragment thereof is administered with one or more additional therapeutic compositions.

51. The method of arrangement 50, wherein the one or more additional therapeutic compositions comprise a cholinesterase inhibitor, an NMDA receptor antagonist, insulin, or any combination thereof.

52. The method of arrangement 51, wherein the cholinesterase inhibitor comprises tacrine, rivastigmine, galantamine, donepezil or any combination thereof.

53. The method of arrangement 51 or 52, wherein the NMDA receptor antagonist comprises memantine.

54. A method of administering an antibody to a subject, comprising:

administering to the subject an anti-Gal3 antibody or binding fragment thereof.

55. The method of arrangement 54, further comprising selecting the subject as having a neurological disease or a proteopathy or at risk of contracting the neurological disease or the proteopathy prior to the administering step.

56. The method of arrangement 54 or 55, wherein the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof.

57. The method of any one of arrangements 54-56, wherein the neurological disorder is Alzheimer's disease.

58. The method of any one of arrangements 54-57, wherein the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof.

59. The method of any one of arrangements 54-58, wherein more than one anti-Gal3 antibody or binding fragment thereof is administered to the subject.

60. A method of promoting neuronal regeneration in a subject in need thereof, comprising:

administering to the subject an effective amount of an anti-Gal3 antibody or binding fragment thereof, thereby promoting neuronal regeneration in the subject.

61. The method of arrangement 60, further comprising selecting the subject as having neuronal degeneration or at risk of having neuronal degeneration prior to the administering step.

62. The method of arrangement 60 or 61, further comprising detecting the neuronal regeneration in the subject after the administering step.

63. The method of any one of arrangements 60 or 62, wherein the subject comprises neuronal degeneration associated with inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof.

64. The method of arrangement 63, wherein the neuronal degeneration is associated with Alzheimer's disease, and wherein the anti-Gal3 antibody or binding fragment thereof disrupts binding between Gal3 and amyloid precursor protein (APP) or amyloid beta (Aβ), or both.

65. The method of any one of arrangements 60-64, wherein more than one anti-Gal3 antibody or binding fragment thereof is administered to the subject.

66. The method of any one of arrangements 11-65, wherein the anti-Gal3 antibody or binding fragment thereof is administered enterally, orally, intranasally, parenterally, intracranially, subcutaneously, intramuscularly, intradermally, or intravenously, or any combination thereof.

67. The method of any one of arrangements 11-66, wherein the anti-Gal3 antibody or binding fragment thereof binds to one or more peptides of SEQ ID NOs: 3-26.

68. The method of any one of arrangements 11-67, wherein the anti-Gal3 antibody or binding fragment thereof binds to the N-terminal domain of Gal3, N-terminus of Gal3, or the tandem repeat domain (TRD) of Gal3.

69. The method of any one of arrangements 11-68, wherein the anti-Gal3 antibody or binding fragment thereof comprising (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3; wherein

the V_{H}-CDR1 comprises an amino acid sequence selected from SEQ ID NOs: 27-44, 245-256, 588-615,

the V_{H}-CDR2 comprises an amino acid sequence selected from SEQ ID NOs: 45-60, 247-248, 616-643,

the V_{H}-CDR3 comprises an amino acid sequence selected from SEQ ID NOs: 61-81, 249-250, 644-671,

the V_{L}-CDR1 comprises an amino acid sequence selected from SEQ ID NOs: 82-101, 251-252, 672-699,

the V_{L}-CDR2 comprises an amino acid sequence selected from SEQ ID NOs: 102-116, 253, 700-727, and

the V_{L}-CDR3 comprises an amino acid sequence selected from SEQ ID NOs: 117-135, 254-255, 728-755.

70. The method of any one of arrangements 11-69, wherein the heavy chain variable region comprises an amino acid sequence having at least 75%, 80%, 85%, 90%, 95%, or 100% sequence identity to any sequence according to SEQ ID NOs: 136-160, 256-257, 756-783.

71. The method of any one of arrangements 11-70, wherein the heavy chain variable region is selected from the group consisting of SEQ ID NOs: 136-160, 256-257, 756-783.

72. The method of any one of arrangements 11-71, wherein the light chain variable region comprises an amino acid sequence having at least 75%, 80%, 85%, 90%, 95%, or 100% sequence identity to any sequence according to SEQ ID NOs: 161-187, 258-259, 784-811.

73. The method of any one of arrangements 11-72, wherein the light chain variable region is selected from the group consisting of SEQ ID NOs: 161-187, 258-259, 784-811.

74. The method of any one of arrangements 11-73, wherein the anti-Gal3 antibody or binding fragment thereof comprises:

1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161;

2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162;

3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163;

4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164;

5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165;

6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166;

7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167;

8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168;

9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169;

10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170;

11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171;

12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172;

13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173;

14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175;

16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176;

17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177;

18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178;

19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179;

20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180;

21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181;

22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182;

23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183;

24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184;

25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185;

26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186;

27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187;

28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258;

29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259;

30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784;

31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785;

32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786;

33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787;

34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788;

35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789;

36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790;

37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791;

38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792;

39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793;

40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794;

41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795;

42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796;

43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797;

44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798;

45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799;

46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800;

47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801;

48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802;

49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803;

50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804;

51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805;

52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806;

53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807;

54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808;

55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809;

56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or

57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

75. The method of any one of arrangements 11-74, wherein the anti-Gal3 antibody or binding fragment thereof comprises:

1) the heavy chain variable region of SEQ ID NO: 136 and the light chain variable region of SEQ ID NO: 161;

2) the heavy chain variable region of SEQ ID NO: 137 and the light chain variable region of SEQ ID NO: 162;

3) the heavy chain variable region of SEQ ID NO: 138 and the light chain variable region of SEQ ID NO: 163;

4) the heavy chain variable region of SEQ ID NO: 139 and the light chain variable region of SEQ ID NO: 164;

5) the heavy chain variable region of SEQ ID NO: 140 and the light chain variable region of SEQ ID NO: 165;

6) the heavy chain variable region of SEQ ID NO: 141 and the light chain variable region of SEQ ID NO: 166;

7) the heavy chain variable region of SEQ ID NO: 142 and the light chain variable region of SEQ ID NO: 167;

8) the heavy chain variable region of SEQ ID NO: 143 and the light chain variable region of SEQ ID NO: 168;

9) the heavy chain variable region of SEQ ID NO: 144 and the light chain variable region of SEQ ID NO: 169;

10) the heavy chain variable region of SEQ ID NO: 145 and the light chain variable region of SEQ ID NO: 170;

11) the heavy chain variable region of SEQ ID NO: 139 and the light chain variable region of SEQ ID NO: 171;

12) the heavy chain variable region of SEQ ID NO: 146 and the light chain variable region of SEQ ID NO: 172;

13) the heavy chain variable region of SEQ ID NO: 147 and the light chain variable region of SEQ ID NO: 173;

14) the heavy chain variable region of SEQ ID NO: 148 and the light chain variable region of SEQ ID NO: 174;

15) the heavy chain variable region of SEQ ID NO: 149 and the light chain variable region of SEQ ID NO: 175;

16) the heavy chain variable region of SEQ ID NO: 150 and the light chain variable region of SEQ ID NO: 176;

17) the heavy chain variable region of SEQ ID NO: 151 and the light chain variable region of SEQ ID NO: 177;

18) the heavy chain variable region of SEQ ID NO: 152 and the light chain variable region of SEQ ID NO: 178;

19) the heavy chain variable region of SEQ ID NO: 153 and the light chain variable region of SEQ ID NO: 179;

20) the heavy chain variable region of SEQ ID NO: 154 and the light chain variable region of SEQ ID NO: 180;

21) the heavy chain variable region of SEQ ID NO: 155 and the light chain variable region of SEQ ID NO: 181;

22) the heavy chain variable region of SEQ ID NO: 156 and the light chain variable region of SEQ ID NO: 182;

23) the heavy chain variable region of SEQ ID NO: 157 and the light chain variable region of SEQ ID NO: 183;

24) the heavy chain variable region of SEQ ID NO: 155 and the light chain variable region of SEQ ID NO: 184;

25) the heavy chain variable region of SEQ ID NO: 158 and the light chain variable region of SEQ ID NO: 185;

26) the heavy chain variable region of SEQ ID NO: 159 and the light chain variable region of SEQ ID NO: 186;

27) the heavy chain variable region of SEQ ID NO: 160 and the light chain variable region of SEQ ID NO: 187

28) the heavy chain variable region of SEQ ID NO: 256 and the light chain variable region of SEQ ID NO: 258;

29) the heavy chain variable region of SEQ ID NO: 257 and the light chain variable region of SEQ ID NO: 259;

30) the heavy chain variable region of SEQ ID NO: 756 and the light chain variable region of SEQ ID NO: 784;

31) the heavy chain variable region of SEQ ID NO: 757 and the light chain variable region of SEQ ID NO: 785;

32) the heavy chain variable region of SEQ ID NO: 758 and the light chain variable region of SEQ ID NO: 786;

33) the heavy chain variable region of SEQ ID NO: 759 and the light chain variable region of SEQ ID NO: 787;

34) the heavy chain variable region of SEQ ID NO: 760 and the light chain variable region of SEQ ID NO: 788;

35) the heavy chain variable region of SEQ ID NO: 761 and the light chain variable region of SEQ ID NO: 789;

36) the heavy chain variable region of SEQ ID NO: 762 and the light chain variable region of SEQ ID NO: 790;

37) the heavy chain variable region of SEQ ID NO: 763 and the light chain variable region of SEQ ID NO: 791;

38) the heavy chain variable region of SEQ ID NO: 764 and the light chain variable region of SEQ ID NO: 792;

39) the heavy chain variable region of SEQ ID NO: 765 and the light chain variable region of SEQ ID NO: 793;

40) the heavy chain variable region of SEQ ID NO: 766 and the light chain variable region of SEQ ID NO: 794;

41) the heavy chain variable region of SEQ ID NO: 767 and the light chain variable region of SEQ ID NO: 795;

42) the heavy chain variable region of SEQ ID NO: 768 and the light chain variable region of SEQ ID NO: 796;

43) the heavy chain variable region of SEQ ID NO: 769 and the light chain variable region of SEQ ID NO: 797;

44) the heavy chain variable region of SEQ ID NO: 770 and the light chain variable region of SEQ ID NO: 798;

45) the heavy chain variable region of SEQ ID NO: 771 and the light chain variable region of SEQ ID NO: 799;

46) the heavy chain variable region of SEQ ID NO: 772 and the light chain variable region of SEQ ID NO: 800;

47) the heavy chain variable region of SEQ ID NO: 773 and the light chain variable region of SEQ ID NO: 801;

48) the heavy chain variable region of SEQ ID NO: 774 and the light chain variable region of SEQ ID NO: 802;

49) the heavy chain variable region of SEQ ID NO: 775 and the light chain variable region of SEQ ID NO: 803;

50) the heavy chain variable region of SEQ ID NO: 776 and the light chain variable region of SEQ ID NO: 804;

51) the heavy chain variable region of SEQ ID NO: 777 and the light chain variable region of SEQ ID NO: 805;

52) the heavy chain variable region of SEQ ID NO: 778 and the light chain variable region of SEQ ID NO: 806;

53) the heavy chain variable region of SEQ ID NO: 779 and the light chain variable region of SEQ ID NO: 807;

54) the heavy chain variable region of SEQ ID NO: 780 and the light chain variable region of SEQ ID NO: 808;

55) the heavy chain variable region of SEQ ID NO: 781 and the light chain variable region of SEQ ID NO: 809;

56) the heavy chain variable region of SEQ ID NO: 782 and the light chain variable region of SEQ ID NO: 810; or

57) the heavy chain variable region of SEQ ID NO: 783 and the light chain variable region of SEQ ID NO: 811.

76. The method of any one of arrangements 11-75, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

77. The method of any one of arrangements 11-76, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.4E11, 847.11D6, 847.20H7, 847.21B11, 849.1D2, 849.2D7, 849.2F12, 849.4B2, 849.4F2, or a binding fragment thereof.

78. The method of any one of arrangements 11-77, wherein the anti-Gal3 antibody or binding fragment thereof is selected from the group consisting of TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof.

79. An anti-Gal3 antibody or binding fragment thereof for use in the treatment of a neurodegenerative disorder in a subject in need thereof.

80. The use of arrangement 79, wherein the neurodegenerative disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof.

81. The use of arrangement 79 or 80, wherein the neurological disorder is Alzheimer's disease, and wherein the anti-Gal3 antibody or binding fragment thereof disrupts binding between Gal3 and amyloid precursor protein (APP) or Aβ, or both.

82. The use of arrangement 81, wherein the APP comprises the sequence of APP695 (SEQ ID NO: 2).

83. The use of arrangement 81 or 82, wherein the Aβ comprises Aβ monomers, Aβ oligomers, Aβ fibrils, or any combination thereof.

84. The use of any one of arrangements 81-83, wherein the Aβ comprises the sequence of Aβ42 (SEQ ID NO: 244).

85. The use of any one of arrangements 81-84, wherein the anti-Gal3 antibody or binding fragment thereof reduces the binding between Gal3 and APP or Aβ, or both, by at least 50%, 55%, 60%, 65%, 70%, 75%, 80%, 85%, 90%, 95%, 96%, 97%, 98%, or 99%, or any percentage within a range defined by any two aforementioned percentages.

86. The use of any one of arrangements 79-85, wherein the anti-Gal3 antibody or binding fragment thereof promotes phagocytic function of microglia in the subject.

87. The use of any one of arrangements 79-86, wherein the anti-Gal3 antibody or binding fragment thereof inhibits AD-mediated activation of microglia in the subject.

88. The use of arrangement 87, wherein the AD-mediated activation of microglia is inhibited by at least 50%, 55%, 60%, 65%, 70%, 75%, 80%, 85%, 90%, 95%, 96%, 97%, 98%, or 99%, or any percentage within a range defined by any two aforementioned percentages.

89. The use of any one of arrangements 79-88, wherein the anti-Gal3 antibody or binding fragment thereof inhibits Aβ fibril or oligomer formation in the subject.

90. The use of arrangement 89, wherein the Aβ fibril or oligomer formation is inhibited by at least 50%, 55%, 60%, 65%, 70%, 75%, 80%, 85%, 90%, 95%, 96%, 97%, 98%, or 99%, or any percentage within a range defined by any two aforementioned percentages.

91. The use of any one of arrangements 79-90, wherein the anti-Gal3 antibody or binding fragment thereof disrupts an interaction between Gal3 and Toll-like receptor 4 (TLR4) or triggering receptor expressed on myeloid cells 2 (TREM2), or both.

92. The use of arrangement 91, wherein the interaction between Gal3 and TLR4 or TREM2, or both is disrupted by at least 50%, 55%, 60%, 65%, 70%, 75%, 80%, 85%, 90%, 95%, 96%, 97%, 98%, or 99%, or any percentage within a range defined by any two aforementioned percentages.

93. An anti-Gal3 antibody or binding fragment thereof for use in the treatment of a proteopathy in a subject in need thereof.

94. The use of arrangement 93, wherein the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof.

95. An anti-Gal3 antibody or binding fragment thereof for use in promoting neuronal regeneration in a subject in need thereof.

96. The use of arrangement 95, wherein the subject comprises neuronal degeneration associated with inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof.

97. The use of arrangement 96, wherein the neuronal degeneration is associated with Alzheimer's disease, and wherein the anti-Gal3 antibody or binding fragment thereof disrupts binding between Gal3 and amyloid precursor protein (APP) or amyloid beta (Aβ), or both, in the subject.

98. The use of any one of arrangements 95-97, wherein more than one anti-Gal3 antibody or binding fragment thereof is administered to the subject.

99. The use of any one of arrangements 79-98, wherein the anti-Gal3 antibody or binding fragment thereof binds to one or more peptides of SEQ ID NOs: 3-26.

100. The use of any one of arrangements 79-99, wherein the anti-Gal3 antibody or binding fragment thereof binds to the N-terminal domain of Gal3, N-terminus of Gal3, or the tandem repeat domain (TRD) of Gal3.

101. The use of any one of arrangements 79-100, wherein the anti-Gal3 antibody or binding fragment thereof comprising (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3; wherein

the V_{H}-CDR1 comprises an amino acid sequence selected from SEQ ID NOs: 27-44, 245-246, 588-615,

the V_{H}-CDR2 comprises an amino acid sequence selected from SEQ ID NOs: 45-60, 247-248, 616-643,

the V_{H}-CDR3 comprises an amino acid sequence selected from SEQ ID NOs: 61-81, 249-250, 644-671,

the V_{L}-CDR1 comprises an amino acid sequence selected from SEQ ID NOs: 82-101, 251-252, 672-699,

the V_{L}-CDR2 comprises an amino acid sequence selected from SEQ ID NOs: 102-116, 253, 700-727, and

the V_{L}-CDR3 comprises an amino acid sequence selected from SEQ ID NOs: 117-135, 254-255, 728-755.

102. The use of any one of arrangements 79-101, wherein the heavy chain variable region comprises an amino acid sequence having at least 75%, 80%, 85%, 90%, 95%, or 100% sequence identity to any sequence according to SEQ ID NOs: 136-160, 256-257, 756-783.

103. The use of any one of arrangements 79-102, wherein the heavy chain variable region is selected from the group consisting of SEQ ID NOs: 136-160, 256-257, 756-783.

104. The use of any one of arrangements 79-103, wherein the light chain variable region comprises an amino acid sequence having at least 75%, 80%, 85%, 90%, 95%, or 100% sequence identity to any sequence according to SEQ ID NOs: 161-187, 258-259, 784-811.

105. The use of any one of arrangements 79-104, wherein the light chain variable region is selected from the group consisting of SEQ ID NOs: 161-187, 258-259 784-811.

106. The use of any one of arrangements 79-105, wherein the anti-Gal3 antibody or binding fragment thereof comprises:

1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161;

2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162;

3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163;

4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164;

5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165;

6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166;

7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167;

8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168;

9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169;

10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170;

11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171;

12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172;

13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173;

14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175;

16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176;

17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177;

18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178;

19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179;

20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180;

21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181;

22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182;

23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183;

24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184;

25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185;

26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186;

27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187;

28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258;

29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259;

30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784;

31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785;

32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786;

33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787;

34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788;

35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789;

36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790;

37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791;

38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792;

39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793;

40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794;

41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795;

42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796;

43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797;

44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798;

45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799;

46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800;

47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801;

48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802;

49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803;

50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804;

51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805;

52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806;

53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807;

54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808;

55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809;

56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or

57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

107. The use of any one of arrangements 79-106, wherein the anti-Gal3 antibody or binding fragment thereof comprises:

1) the heavy chain variable region of SEQ ID NO: 136 and the light chain variable region of SEQ ID NO: 161;

2) the heavy chain variable region of SEQ ID NO: 137 and the light chain variable region of SEQ ID NO: 162;

3) the heavy chain variable region of SEQ ID NO: 138 and the light chain variable region of SEQ ID NO: 163;

4) the heavy chain variable region of SEQ ID NO: 139 and the light chain variable region of SEQ ID NO: 164;

5) the heavy chain variable region of SEQ ID NO: 140 and the light chain variable region of SEQ ID NO: 165;

6) the heavy chain variable region of SEQ ID NO: 141 and the light chain variable region of SEQ ID NO: 166;

7) the heavy chain variable region of SEQ ID NO: 142 and the light chain variable region of SEQ ID NO: 167;

8) the heavy chain variable region of SEQ ID NO: 143 and the light chain variable region of SEQ ID NO: 168;

9) the heavy chain variable region of SEQ ID NO: 144 and the light chain variable region of SEQ ID NO: 169;

10) the heavy chain variable region of SEQ ID NO: 145 and the light chain variable region of SEQ ID NO: 170;

11) the heavy chain variable region of SEQ ID NO: 139 and the light chain variable region of SEQ ID NO: 171;

12) the heavy chain variable region of SEQ ID NO: 146 and the light chain variable region of SEQ ID NO: 172;

13) the heavy chain variable region of SEQ ID NO: 147 and the light chain variable region of SEQ ID NO: 173;

14) the heavy chain variable region of SEQ ID NO: 148 and the light chain variable region of SEQ ID NO: 174;

15) the heavy chain variable region of SEQ ID NO: 149 and the light chain variable region of SEQ ID NO: 175;

16) the heavy chain variable region of SEQ ID NO: 150 and the light chain variable region of SEQ ID NO: 176;

17) the heavy chain variable region of SEQ ID NO: 151 and the light chain variable region of SEQ ID NO: 177;

18) the heavy chain variable region of SEQ ID NO: 152 and the light chain variable region of SEQ ID NO: 178;

19) the heavy chain variable region of SEQ ID NO: 153 and the light chain variable region of SEQ ID NO: 179;

20) the heavy chain variable region of SEQ ID NO: 154 and the light chain variable region of SEQ ID NO: 180;

21) the heavy chain variable region of SEQ ID NO: 155 and the light chain variable region of SEQ ID NO: 181;

22) the heavy chain variable region of SEQ ID NO: 156 and the light chain variable region of SEQ ID NO: 182;

23) the heavy chain variable region of SEQ ID NO: 157 and the light chain variable region of SEQ ID NO: 183;

24) the heavy chain variable region of SEQ ID NO: 155 and the light chain variable region of SEQ ID NO: 184;

25) the heavy chain variable region of SEQ ID NO: 158 and the light chain variable region of SEQ ID NO: 185;

26) the heavy chain variable region of SEQ ID NO: 159 and the light chain variable region of SEQ ID NO: 186;

27) the heavy chain variable region of SEQ ID NO: 160 and the light chain variable region of SEQ ID NO: 187;

28) the heavy chain variable region of SEQ ID NO: 256 and the light chain variable region of SEQ ID NO: 258;

29) the heavy chain variable region of SEQ ID NO: 257 and the light chain variable region of SEQ ID NO: 259;

30) the heavy chain variable region of SEQ ID NO: 756 and the light chain variable region of SEQ ID NO: 784;

31) the heavy chain variable region of SEQ ID NO: 757 and the light chain variable region of SEQ ID NO: 785;

32) the heavy chain variable region of SEQ ID NO: 758 and the light chain variable region of SEQ ID NO: 786;

33) the heavy chain variable region of SEQ ID NO: 759 and the light chain variable region of SEQ ID NO: 787;

34) the heavy chain variable region of SEQ ID NO: 760 and the light chain variable region of SEQ ID NO: 788;

35) the heavy chain variable region of SEQ ID NO: 761 and the light chain variable region of SEQ ID NO: 789;

36) the heavy chain variable region of SEQ ID NO: 762 and the light chain variable region of SEQ ID NO: 790;

37) the heavy chain variable region of SEQ ID NO: 763 and the light chain variable region of SEQ ID NO: 791;

38) the heavy chain variable region of SEQ ID NO: 764 and the light chain variable region of SEQ ID NO: 792;

39) the heavy chain variable region of SEQ ID NO: 765 and the light chain variable region of SEQ ID NO: 793;

40) the heavy chain variable region of SEQ ID NO: 766 and the light chain variable region of SEQ ID NO: 794;

41) the heavy chain variable region of SEQ ID NO: 767 and the light chain variable region of SEQ ID NO: 795;

42) the heavy chain variable region of SEQ ID NO: 768 and the light chain variable region of SEQ ID NO: 796;

43) the heavy chain variable region of SEQ ID NO: 769 and the light chain variable region of SEQ ID NO: 797;

44) the heavy chain variable region of SEQ ID NO: 770 and the light chain variable region of SEQ ID NO: 798;

45) the heavy chain variable region of SEQ ID NO: 771 and the light chain variable region of SEQ ID NO: 799;

46) the heavy chain variable region of SEQ ID NO: 772 and the light chain variable region of SEQ ID NO: 800;

47) the heavy chain variable region of SEQ ID NO: 773 and the light chain variable region of SEQ ID NO: 801;

48) the heavy chain variable region of SEQ ID NO: 774 and the light chain variable region of SEQ ID NO: 802;

49) the heavy chain variable region of SEQ ID NO: 775 and the light chain variable region of SEQ ID NO: 803;

50) the heavy chain variable region of SEQ ID NO: 776 and the light chain variable region of SEQ ID NO: 804;

51) the heavy chain variable region of SEQ ID NO: 777 and the light chain variable region of SEQ ID NO: 805;

52) the heavy chain variable region of SEQ ID NO: 778 and the light chain variable region of SEQ ID NO: 806;

53) the heavy chain variable region of SEQ ID NO: 779 and the light chain variable region of SEQ ID NO: 807;

54) the heavy chain variable region of SEQ ID NO: 780 and the light chain variable region of SEQ ID NO: 808;

55) the heavy chain variable region of SEQ ID NO: 781 and the light chain variable region of SEQ ID NO: 809;

56) the heavy chain variable region of SEQ ID NO: 782 and the light chain variable region of SEQ ID NO: 810; or 57) the heavy chain variable region of SEQ ID NO: 783 and the light chain variable region of SEQ ID NO: 811.

108. The use of any one of arrangements 79-107, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

109. The use of any one of arrangements 79-108, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.4E11, 847.11D6, 847.20H7, 847.21B11, 849.1D2, 849.2D7, 849.2F12, 849.4B2, 849.4F2, or a binding fragment thereof.

110. The use of any one of arrangements 79-109, wherein the anti-Gal3 antibody or binding fragment thereof is selected from the group consisting of TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof.

111. The use of any one of arrangements 79-110, wherein the anti-Gal3 antibody or binding fragment thereof is administered enterally, orally, intranasally, parenterally, intracranially, subcutaneously, intramuscularly, intradermally, or intravenously, or any combination thereof.

112. The method of any one of arrangements 11-78, wherein the anti-Gal3 antibody or binding fragment thereof is able to cross the blood-brain barrier.

113. The use of any one of arrangements 79-111, wherein the anti-Gal3 antibody or binding fragment thereof is able to cross the blood-brain barrier.

114. An antibody conjugate comprising:

an anti-Gal3 antibody or binding fragment thereof; and

a payload conjugated to the anti-Gal3 antibody or binding fragment thereof, wherein

the antibody conjugate is able to cross a blood-brain barrier.

115. The antibody conjugate of arrangement 114, wherein the payload is not independently capable of crossing the blood-brain barrier or has low permeability across the blood-brain barrier without being conjugated to the anti-Gal3 antibody or binding fragment thereof.

116. The antibody conjugate of arrangement 114 or 115, wherein conjugation of the payload to the anti-Gal3 antibody or binding fragment thereof increases the permeability of the payload across the blood-brain barrier by at least 5%, 10%, 20%, 30%, 40%, 50%, 60%, 70%, 80%, 90%, 100%, 200%, 300%, 400%, or 500%, or any increase within a range defined by any two of the aforementioned percentages, compared to the unconjugated payload.

117. The antibody conjugate of any one of arrangements 114-116, wherein the permeability of the payload across the blood-brain barrier is less than 95%, 90%, 80%, 70%, 60%, 50%, 40%, 30%, 20%, 10%, 9%, 8%, 7%, 6%, 5%, 4%, 3%, 2%, or 1% of the permeability of the antibody conjugate across the blood-brain barrier.

118. The antibody conjugate of any one of arrangements 114-117, wherein the payload or the anti-Gal3 antibody or binding fragment thereof, or both, is used to treat a neurological disorder that is treated in the brain.

119. The antibody conjugate of any one of arrangements 114-118, wherein the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or any combination thereof.

120. The antibody conjugate of any one of arrangements 114-119, wherein the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, protein, enzyme, or any combination thereof.

121. The antibody conjugate of any one of arrangements 114-120, wherein the payload is a second antibody.

122. The antibody conjugate of arrangement 121, wherein the second antibody is not independently capable of crossing the blood-brain barrier or has low permeability across the blood-brain barrier without being conjugated to the anti-Gal3 antibody or binding fragment thereof.

123. The antibody conjugate of any one of arrangements 114-122, wherein the blood-brain barrier is a mammalian blood-brain barrier.

124. The antibody conjugate of any one of arrangements 114-123, wherein the blood-brain barrier is a human blood-brain barrier.

125. The antibody conjugate of any one of arrangements 114-124, wherein the antibody conjugate is formulated to be administered enterally, orally, intranasally, parenterally, intracranially, subcutaneously, intramuscularly, intradermally, or intravenously, or any combination thereof.

126. The antibody conjugate of any one of arrangements 114-125, wherein the anti-Gal3 antibody or binding fragment thereof binds to one or more peptides of SEQ ID NOs: 3-26.

127. The antibody conjugate of any one of arrangements 114-126, wherein the anti-Gal3 antibody or binding fragment thereof binds to the N-terminal domain of Gal3, N-terminus of Gal3, or the tandem repeat domain (TRD) of Gal3.

128. The antibody conjugate of any one of arrangements 114-127, wherein the anti-Gal3 antibody or binding fragment thereof belongs to bin 3, 8, 17, or 24.

129. The antibody conjugate of any one of arrangements 114-128, wherein the anti-Gal3 antibody or binding fragment thereof disrupts an interaction between Gal3 and an antibody that belongs to bin 3, 8, 17 or 24.

130. The antibody conjugate of any one of arrangements 114-129, wherein the anti-Gal3 antibody or binding fragment thereof comprising (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3; wherein

the V_{H}-CDR1 comprises an amino acid sequence selected from SEQ ID NOs: 27-44, 245-246, 588-615,

the V_{H}-CDR2 comprises an amino acid sequence selected from SEQ ID NOs: 45-60, 247-248, 616-643,

the V_{H}-CDR3 comprises an amino acid sequence selected from SEQ ID NOs: 61-81, 249-250, 644-671,

the V_{L}-CDR1 comprises an amino acid sequence selected from SEQ ID NOs: 82-101, 251-252, 672-699,

the V_{L}-CDR2 comprises an amino acid sequence selected from SEQ ID NOs: 102-116, 253, 700-727, and

the V_{L}-CDR3 comprises an amino acid sequence selected from SEQ ID NOs: 117-135, 254-255, 728-755.

131. The antibody conjugate of any one of arrangements 114-130, wherein the heavy chain variable region comprises an amino acid sequence having at least 75%, 80%, 85%, 90%, 95%, or 100% sequence identity to any sequence according to SEQ ID NOs: 136-160, 256-257, 756-783.

132. The antibody conjugate of any one of arrangements 114-131, wherein the heavy chain variable region is selected from the group consisting of SEQ ID NOs: 136-160, 256-257, 756-783.

133. The antibody conjugate of any one of arrangements 114-132, wherein the light chain variable region comprises an amino acid sequence having at least 75%, 80%, 85%, 90%, 95%, or 100% sequence identity to any sequence according to SEQ ID NOs: 161-187, 258-259, 784-811.

134. The antibody conjugate of any one of arrangements 114-133, wherein the light chain variable region is selected from the group consisting of SEQ ID NOs: 161-187, 258-259, 784-811.

135. The antibody conjugate of any one of arrangements 114-134, wherein the anti-Gal3 antibody or binding fragment thereof comprises:

1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161;

2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162;

3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163;

4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164;

5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165;

6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166;

7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167;

8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168;

9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169;

10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170;

11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171;

12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172;

13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173;

14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175;

16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176;

17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177;

18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178;

19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179;

20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180;

21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181;

22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182;

23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183;

24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184;

25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185;

26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186;

27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187;

28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258;

29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259;

30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784;

31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785;

32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786;

33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787;

34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788;

35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789;

36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790;

37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791;

38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792;

39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793;

40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794;

41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795;

42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796;

43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797;

44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798;

45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799;

46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800;

47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801;

48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802;

49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803;

50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804;

51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805;

52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806;

53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807;

54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808;

55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809;

56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or

57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

136. The antibody conjugate of any one of arrangements 114-135, wherein the anti-Gal3 antibody or binding fragment thereof comprises:

1) the heavy chain variable region of SEQ ID NO: 136 and the light chain variable region of SEQ ID NO: 161;

2) the heavy chain variable region of SEQ ID NO: 137 and the light chain variable region of SEQ ID NO: 162;

3) the heavy chain variable region of SEQ ID NO: 138 and the light chain variable region of SEQ ID NO: 163;

4) the heavy chain variable region of SEQ ID NO: 139 and the light chain variable region of SEQ ID NO: 164;

5) the heavy chain variable region of SEQ ID NO: 140 and the light chain variable region of SEQ ID NO: 165;

6) the heavy chain variable region of SEQ ID NO: 141 and the light chain variable region of SEQ ID NO: 166;

7) the heavy chain variable region of SEQ ID NO: 142 and the light chain variable region of SEQ ID NO: 167;

8) the heavy chain variable region of SEQ ID NO: 143 and the light chain variable region of SEQ ID NO: 168;

9) the heavy chain variable region of SEQ ID NO: 144 and the light chain variable region of SEQ ID NO: 169;

10) the heavy chain variable region of SEQ ID NO: 145 and the light chain variable region of SEQ ID NO: 170;

11) the heavy chain variable region of SEQ ID NO: 139 and the light chain variable region of SEQ ID NO: 171;

12) the heavy chain variable region of SEQ ID NO: 146 and the light chain variable region of SEQ ID NO: 172;

13) the heavy chain variable region of SEQ ID NO: 147 and the light chain variable region of SEQ ID NO: 173;

14) the heavy chain variable region of SEQ ID NO: 148 and the light chain variable region of SEQ ID NO: 174;

15) the heavy chain variable region of SEQ ID NO: 149 and the light chain variable region of SEQ ID NO: 175;

16) the heavy chain variable region of SEQ ID NO: 150 and the light chain variable region of SEQ ID NO: 176;

17) the heavy chain variable region of SEQ ID NO: 151 and the light chain variable region of SEQ ID NO: 177;

18) the heavy chain variable region of SEQ ID NO: 152 and the light chain variable region of SEQ ID NO: 178;

19) the heavy chain variable region of SEQ ID NO: 153 and the light chain variable region of SEQ ID NO: 179;

20) the heavy chain variable region of SEQ ID NO: 154 and the light chain variable region of SEQ ID NO: 180;

21) the heavy chain variable region of SEQ ID NO: 155 and the light chain variable region of SEQ ID NO: 181;

22) the heavy chain variable region of SEQ ID NO: 156 and the light chain variable region of SEQ ID NO: 182;

23) the heavy chain variable region of SEQ ID NO: 157 and the light chain variable region of SEQ ID NO: 183;

24) the heavy chain variable region of SEQ ID NO: 155 and the light chain variable region of SEQ ID NO: 184;

25) the heavy chain variable region of SEQ ID NO: 158 and the light chain variable region of SEQ ID NO: 185;

26) the heavy chain variable region of SEQ ID NO: 159 and the light chain variable region of SEQ ID NO: 186;

27) the heavy chain variable region of SEQ ID NO: 160 and the light chain variable region of SEQ ID NO: 187

28) the heavy chain variable region of SEQ ID NO: 256 and the light chain variable region of SEQ ID NO: 258;

29) the heavy chain variable region of SEQ ID NO: 257 and the light chain variable region of SEQ ID NO: 259;

30) the heavy chain variable region of SEQ ID NO: 756 and the light chain variable region of SEQ ID NO: 784;

31) the heavy chain variable region of SEQ ID NO: 757 and the light chain variable region of SEQ ID NO: 785;

32) the heavy chain variable region of SEQ ID NO: 758 and the light chain variable region of SEQ ID NO: 786;

33) the heavy chain variable region of SEQ ID NO: 759 and the light chain variable region of SEQ ID NO: 787;

34) the heavy chain variable region of SEQ ID NO: 760 and the light chain variable region of SEQ ID NO: 788;

35) the heavy chain variable region of SEQ ID NO: 761 and the light chain variable region of SEQ ID NO: 789;

36) the heavy chain variable region of SEQ ID NO: 762 and the light chain variable region of SEQ ID NO: 790;

37) the heavy chain variable region of SEQ ID NO: 763 and the light chain variable region of SEQ ID NO: 791;

38) the heavy chain variable region of SEQ ID NO: 764 and the light chain variable region of SEQ ID NO: 792;

39) the heavy chain variable region of SEQ ID NO: 765 and the light chain variable region of SEQ ID NO: 793;

40) the heavy chain variable region of SEQ ID NO: 766 and the light chain variable region of SEQ ID NO: 794;

41) the heavy chain variable region of SEQ ID NO: 767 and the light chain variable region of SEQ ID NO: 795;

42) the heavy chain variable region of SEQ ID NO: 768 and the light chain variable region of SEQ ID NO: 796;

43) the heavy chain variable region of SEQ ID NO: 769 and the light chain variable region of SEQ ID NO: 797;

44) the heavy chain variable region of SEQ ID NO: 770 and the light chain variable region of SEQ ID NO: 798;

45) the heavy chain variable region of SEQ ID NO: 771 and the light chain variable region of SEQ ID NO: 799;

46) the heavy chain variable region of SEQ ID NO: 772 and the light chain variable region of SEQ ID NO: 800;

47) the heavy chain variable region of SEQ ID NO: 773 and the light chain variable region of SEQ ID NO: 801;

48) the heavy chain variable region of SEQ ID NO: 774 and the light chain variable region of SEQ ID NO: 802;

49) the heavy chain variable region of SEQ ID NO: 775 and the light chain variable region of SEQ ID NO: 803;

50) the heavy chain variable region of SEQ ID NO: 776 and the light chain variable region of SEQ ID NO: 804;

51) the heavy chain variable region of SEQ ID NO: 777 and the light chain variable region of SEQ ID NO: 805;

52) the heavy chain variable region of SEQ ID NO: 778 and the light chain variable region of SEQ ID NO: 806;

53) the heavy chain variable region of SEQ ID NO: 779 and the light chain variable region of SEQ ID NO: 807;

54) the heavy chain variable region of SEQ ID NO: 780 and the light chain variable region of SEQ ID NO: 808;

55) the heavy chain variable region of SEQ ID NO: 781 and the light chain variable region of SEQ ID NO: 809;

56) the heavy chain variable region of SEQ ID NO: 782 and the light chain variable region of SEQ ID NO: 810; or

57) the heavy chain variable region of SEQ ID NO: 783 and the light chain variable region of SEQ ID NO: 811.

137. The antibody conjugate of any one of arrangements 114-136, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

138. The antibody conjugate of any one of arrangements 114-137, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.4E11, 847.11D6, 847.20H7, 847.21B11, 849.1D2, 849.2D7, 849.2F12, 849.4B2, 849.4F2, or a binding fragment thereof.

139. The antibody conjugate of any one of arrangements 114-138, wherein the anti-Gal3 antibody or binding fragment thereof is selected from the group consisting of TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof.

140. The antibody conjugate of any one of arrangements 114-139, wherein the anti-Gal3 antibody or binding fragment thereof disrupts an interaction between Gal3 and an antibody selected from 846.4D5, 15F10.2D6, F846C.1B2, and F846C.1H12.

141. A multi-specific antibody comprising a first binding domain that binds to Gal3 and a second binding domain that binds to a therapeutic target molecule located in the brain of a subject.

142. The multi-specific antibody of arrangement 141, wherein the second binding domain is not independently capable of crossing the blood-brain barrier or has low permeability across the blood-brain barrier without being conjugated to the anti-Gal3 antibody or binding fragment thereof.

143. The multi-specific antibody of arrangement 141 or 142, wherein the permeability of the second binding domain across the blood-brain barrier is less than 95%, 90%, 80%, 70%, 60%, 50%, 40%, 30%, 20%, 10%, 9%, 8%, 7%, 6%, 5%, 4%, 3%, 2%, or 1% of the permeability of the multi-specific antibody across the blood-brain barrier.

144. The multi-specific antibody of any one of arrangements 141-143, wherein the first binding domain that binds to Gal3 belongs to bin 3, 8, 17, or 24.

145. The multi-specific antibody of any one of arrangements 141-144, wherein the first binding domain that binds to Gal3 disrupts an interaction between Gal3 and an antibody that belongs to bin 3, 8, 17 or 24.

146. The multi-specific antibody of any one of arrangements 141-145, wherein the first binding domain that binds to Gal3 competes with an antibody that belongs to bins 3, 8, 17 or 24 for binding to Gal3.

147. The multi-specific antibody of any one of arrangements 141-146, wherein the first binding domain that binds to Gal3 is a binding domain of the anti-Gal3 antibody or binding fragment thereof of any one of the antibody conjugates of arrangements 114-140.

148. A pharmaceutical composition comprising the antibody conjugate of any one of arrangements 114-140 or the multi-specific antibody of any one of arrangements 141-147 and at least one pharmaceutically acceptable diluent, excipient, or carrier.

149. A method of delivering a payload to the central nervous system of a subject in need thereof, comprising administering to the subject an antibody conjugate comprising an anti-Gal3 antibody or binding fragment thereof and a payload conjugated to the anti-Gal3 antibody or binding fragment thereof, wherein the antibody conjugate is able to cross a blood-brain barrier.

150. A method of increasing the permeability of a payload across the blood-brain barrier of a subject in need thereof, comprising conjugating an anti-Gal3 antibody or binding fragment thereof to the payload to form an antibody conjugate.

151. The method of arrangement 150, further comprising administering to the subject the antibody conjugate.

152. The method of any one of arrangements 149-151, wherein the payload does not normally cross the blood-brain barrier.

153. The method of any one of arrangements 149-152, wherein conjugating the payload to the anti-Gal3 antibody or binding fragment thereof increases the permeability of the payload across the blood-brain barrier by at least 5%, 10%, 20%, 30%, 40%, 50%, 60%, 70%, 80%, 90%, 100%, 200%, 300%, 400%, or 500%, or any increase within a range defined by any two of the aforementioned percentages, compared to the unconjugated payload.

154. The method of any one of arrangements 149-153, wherein the permeability of the payload across the blood-brain barrier is less than 95%, 90%, 80%, 70%, 60%, 50%, 40%, 30%, 20%, 10%, 9%, 8%, 7%, 6%, 5%, 4%, 3%, 2%, or 1% of the permeability of the antibody conjugate across the blood-brain barrier.

155. The method of any one of arrangements 149-154, wherein the payload, or the anti-Gal3 antibody or binding fragment thereof, or both, is used to treat a neurological disorder.

156. The method of arrangement 155, wherein the neurological disorder comprises inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer, or any combination thereof.

157. The method of any one of arrangements 149-156, wherein the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, protein, enzyme, or any combination thereof.

158. The method of any one of arrangements 149-157, wherein the payload is second antibody.

159. The method of any one of arrangements 149-158, wherein the second antibody is not independently capable of crossing the blood-brain barrier or has low permeability across the blood-brain barrier without being conjugated to the anti-Gal3 antibody or binding fragment thereof.

160. The method of any one of arrangements 149-159, wherein the subject is a mammal.

161. The method of any one of arrangements 149-160, wherein the subject is a human.

162. The method of any one of arrangements 149-161, wherein the antibody conjugate is administered enterally, orally, intranasally, parenterally, intracranially, subcutaneously, intramuscularly, intradermally, or intravenously, or any combination thereof.

163. The method of any one of arrangements 149-162, wherein the anti-Gal3 antibody or binding fragment thereof binds to one or more peptides of SEQ ID NOs: 3-26.

164. The method of any one of arrangements 149-163, wherein the anti-Gal3 antibody or binding fragment thereof binds to the N-terminal domain of Gal3, N-terminus of Gal3, or the tandem repeat domain (TRD) of Gal3.

165. The method of any one of arrangements 149-164, wherein the anti-Gal3 antibody or binding fragment thereof belongs to bin 3, 8, 17, or 24.

166. The method of any one of arrangements 149-165, wherein the anti-Gal3 antibody or binding fragment thereof disrupts an interaction between Gal3 and an antibody that belongs to bin 3, 8, 17 or 24.

167. The method of any one of arrangements 149-166, wherein the anti-Gal3 antibody or binding fragment thereof competes with an antibody that belongs to bins 3, 8, 17 or 24 for binding to Gal3.

168. The method of any one of arrangements 149-167, wherein the anti-Gal3 antibody or binding fragment thereof comprising (1) a heavy chain variable region comprising a V_{H}-CDR1, a V_{H}-CDR2, and a V_{H}-CDR3, and (2) a light chain variable region comprising a V_{L}-CDR1, a V_{L}-CDR2, and a V_{L}-CDR3; wherein

the V_{H}-CDR1 comprises an amino acid sequence selected from SEQ ID NOs: 27-44, 245-246, 588-615,

the V_{H}-CDR2 comprises an amino acid sequence selected from SEQ ID NOs: 45-60, 247-248, 616-643,

the V_{H}-CDR3 comprises an amino acid sequence selected from SEQ ID NOs: 61-81, 249-250, 644-671,

the V_{L}-CDR1 comprises an amino acid sequence selected from SEQ ID NOs: 82-101, 251-252, 672-699,

the V_{L}-CDR2 comprises an amino acid sequence selected from SEQ ID NOs: 102-116, 253, 700-727, and

the V_{L}-CDR3 comprises an amino acid sequence selected from SEQ ID NOs: 117-135, 254-255, 728-755.

169. The method of any one of arrangements 149-168, wherein the heavy chain variable region comprises an amino acid sequence having at least 75%, 80%, 85%, 90%, 95%, or 100% sequence identity to any sequence according to SEQ ID NOs: 136-160, 256-257, 756-783.

170. The method of any one of arrangements 149-169, wherein the heavy chain variable region is selected from the group consisting of SEQ ID NOs: 136-160, 256-257, 756-783.

171. The method of any one of arrangements 149-170, wherein the light chain variable region comprises an amino acid sequence having at least 75%, 80%, 85%, 90%, 95%, or 100% sequence identity to any sequence according to SEQ ID NOs: 161-187, 258-259, 784-811.

172. The method of any one of arrangements 149-171, wherein the light chain variable region is selected from the group consisting of SEQ ID NOs: 161-187, 258-259, 784-811.

173. The method of any one of arrangements 149-172, wherein the anti-Gal3 antibody or binding fragment thereof comprises:

1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161;

2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162;

3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163;

4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164;

5) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165;

6) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166;

7) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167;

8) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168;

9) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169;

10) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170;

11) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171;

12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172;

13) the V_{H}-CDR1, V_{H}-CDR2, V_{H}-CDR3 of the V_{H}-CDR1, V_{H}-CDR2, V_{H}-CDR3 within SEQ ID NO: 147 and the V_{L}-CDR1, V_{L}-CDR2, V_{L}-CDR3 of the V_{L}-CDR1, V_{L}-CDR2, V_{L}-CDR3 within SEQ ID NO: 173;

14) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

15) the V_{H}-CDR1, V_{H}-CDR2, V_{H}-CDR3 of the V_{H}-CDR1, V_{H}-CDR2, V_{H}-CDR3 within SEQ ID NO: 149 and the V_{L}-CDR1, V_{L}-CDR2, V_{L}-CDR3 of the V_{L}-CDR1, V_{L}-CDR2, V_{L}-CDR3 within SEQ ID NO: 175;

16) the V_{H}-CDR1, V_{H}-CDR2, V_{H}-CDR3 of the V_{H}-CDR1, V_{H}-CDR2, V_{H}-CDR3 within SEQ ID NO: 150 and the V_{L}-CDR1, V_{L}-CDR2, V_{L}-CDR3 of the V_{L}-CDR1, V_{L}-CDR2, V_{L}-CDR3 within SEQ ID NO: 176;

17) the V_{H}-CDR1, V_{H}-CDR2, V_{H}-CDR3 of the V_{H}-CDR1, V_{H}-CDR2, V_{H}-CDR3 within SEQ ID NO: 151 and the V_{L}-CDR1, V_{L}-CDR2, V_{L}-CDR3 of the V_{L}-CDR1, V_{L}-CDR2, V_{L}-CDR3 within SEQ ID NO: 177;

18) the V_{H}-CDR1, V_{H}-CDR2, V_{H}-CDR3 of the V_{H}-CDR1, V_{H}-CDR2, V_{H}-CDR3 within SEQ ID NO: 152 and the V_{L}-CDR1, V_{L}-CDR2, V_{L}-CDR3 of the V_{L}-CDR1, V_{L}-CDR2, V_{L}-CDR3 within SEQ ID NO: 178;

19) the V_{H}-CDR1, V_{H}-CDR2, V_{H}-CDR3 of the V_{H}-CDR1, V_{H}-CDR2, V_{H}-CDR3 within SEQ ID NO: 153 and the V_{L}-CDR1, V_{L}-CDR2, V_{L}-CDR3 of the V_{L}-CDR1, V_{L}-CDR2, V_{L}-CDR3 within SEQ ID NO: 179;

20) the V_{H}-CDR1, V_{H}-CDR2, V_{H}-CDR3 of the V_{H}-CDR1, V_{H}-CDR2, V_{H}-CDR3 within SEQ ID NO: 154 and the V_{L}-CDR1, V_{L}-CDR2, V_{L}-CDR3 of the V_{L}-CDR1, V_{L}-CDR2, V_{L}-CDR3 within SEQ ID NO: 180;

21) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 181;

22) the V_{H}-CDR1, V_{H}-CDR2, V_{H}-CDR3 of the V_{H}-CDR1, V_{H}-CDR2, V_{H}-CDR3 within SEQ ID NO: 156 and the V_{L}-CDR1, V_{L}-CDR2, V_{L}-CDR3 of the V_{L}-CDR1, V_{L}-CDR2, V_{L}-CDR3 within SEQ ID NO: 182;

23) the V_{H}-CDR1, V_{H}-CDR2, V_{H}-CDR3 of the V_{H}-CDR1, V_{H}-CDR2, V_{H}-CDR3 within SEQ ID NO: 157 and the V_{L}-CDR1, V_{L}-CDR2, V_{L}-CDR3 of the V_{L}-CDR1, V_{L}-CDR2, V_{L}-CDR3 within SEQ ID NO: 183;

24) the V_{H}-CDR1, V_{H}-CDR2, V_{H}-CDR3 of the V_{H}-CDR1, V_{H}-CDR2, V_{H}-CDR3 within SEQ ID NO: 155 and the V_{L}-CDR1, V_{L}-CDR2, V_{L}-CDR3 of the V_{L}-CDR1, V_{L}-CDR2, V_{L}-CDR3 within SEQ ID NO: 184;

25) the V_{H}-CDR1, V_{H}-CDR2, V_{H}-CDR3 of the V_{H}-CDR1, V_{H}-CDR2, V_{H}-CDR3 within SEQ ID NO: 158 and the V_{L}-CDR1, V_{L}-CDR2, V_{L}-CDR3 of the V_{L}-CDR1, V_{L}-CDR2, V_{L}-CDR3 within SEQ ID NO: 185;

26) the V_{H}-CDR1, V_{H}-CDR2, V_{H}-CDR3 of the V_{H}-CDR1, V_{H}-CDR2, V_{H}-CDR3 within SEQ ID NO: 159 and the V_{L}-CDR1, V_{L}-CDR2, V_{L}-CDR3 of the V_{L}-CDR1, V_{L}-CDR2, V_{L}-CDR3 within SEQ ID NO: 186;

27) the V_{H}-CDR1, V_{H}-CDR2, V_{H}-CDR3 of the V_{H}-CDR1, V_{H}-CDR2, V_{H}-CDR3 within SEQ ID NO: 160 and the V_{L}-CDR1, V_{L}-CDR2, V_{L}-CDR3 of the V_{L}-CDR1, V_{L}-CDR2, V_{L}-CDR3 within SEQ ID NO: 187

28) the V_{H}-CDR1, V_{H}-CDR2, V_{H}-CDR3 of the V_{H}-CDR1, V_{H}-CDR2, V_{H}-CDR3 within SEQ ID NO: 256 and the V_{L}-CDR1, V_{L}-CDR2, V_{L}-CDR3 of the V_{L}-CDR1, V_{L}-CDR2, V_{L}-CDR3 within SEQ ID NO: 258;

29) the V_{H}-CDR1, V_{H}-CDR2, V_{H}-CDR3 of the V_{H}-CDR1, V_{H}-CDR2, V_{H}-CDR3 within SEQ ID NO: 257 and the V_{L}-CDR1, V_{L}-CDR2, V_{L}-CDR3 of the V_{L}-CDR1, V_{L}-CDR2, V_{L}-CDR3 within SEQ ID NO: 259;

30) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784;

31) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785;

32) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786;

33) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787;

34) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788;

35) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789;

36) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790;

37) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791;

38) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792;

39) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793;

40) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794;

41) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795;

42) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796;

43) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797;

44) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798;

45) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799;

46) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800;

47) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801;

48) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802;

49) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803;

50) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804;

51) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805;

52) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806;

53) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807;

54) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808;

55) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809;

56) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or

57) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

174. The method of any one of arrangements 149-173, wherein the anti-Gal3 antibody or binding fragment thereof comprises:

1) the heavy chain variable region of SEQ ID NO: 136 and the light chain variable region of SEQ ID NO: 161;

2) the heavy chain variable region of SEQ ID NO: 137 and the light chain variable region of SEQ ID NO: 162;

3) the heavy chain variable region of SEQ ID NO: 138 and the light chain variable region of SEQ ID NO: 163;

4) the heavy chain variable region of SEQ ID NO: 139 and the light chain variable region of SEQ ID NO: 164;

5) the heavy chain variable region of SEQ ID NO: 140 and the light chain variable region of SEQ ID NO: 165;

6) the heavy chain variable region of SEQ ID NO: 141 and the light chain variable region of SEQ ID NO: 166;

7) the heavy chain variable region of SEQ ID NO: 142 and the light chain variable region of SEQ ID NO: 167;

8) the heavy chain variable region of SEQ ID NO: 143 and the light chain variable region of SEQ ID NO: 168;

9) the heavy chain variable region of SEQ ID NO: 144 and the light chain variable region of SEQ ID NO: 169;

10) the heavy chain variable region of SEQ ID NO: 145 and the light chain variable region of SEQ ID NO: 170;

11) the heavy chain variable region of SEQ ID NO: 139 and the light chain variable region of SEQ ID NO: 171;

12) the heavy chain variable region of SEQ ID NO: 146 and the light chain variable region of SEQ ID NO: 172;

13) the heavy chain variable region of SEQ ID NO: 147 and the light chain variable region of SEQ ID NO: 173;

14) the heavy chain variable region of SEQ ID NO: 148 and the light chain variable region of SEQ ID NO: 174;

15) the heavy chain variable region of SEQ ID NO: 149 and the light chain variable region of SEQ ID NO: 175;

16) the heavy chain variable region of SEQ ID NO: 150 and the light chain variable region of SEQ ID NO: 176;

17) the heavy chain variable region of SEQ ID NO: 151 and the light chain variable region of SEQ ID NO: 177;

18) the heavy chain variable region of SEQ ID NO: 152 and the light chain variable region of SEQ ID NO: 178;

19) the heavy chain variable region of SEQ ID NO: 153 and the light chain variable region of SEQ ID NO: 179;

20) the heavy chain variable region of SEQ ID NO: 154 and the light chain variable region of SEQ ID NO: 180;

21) the heavy chain variable region of SEQ ID NO: 155 and the light chain variable region of SEQ ID NO: 181;

22) the heavy chain variable region of SEQ ID NO: 156 and the light chain variable region of SEQ ID NO: 182;

23) the heavy chain variable region of SEQ ID NO: 157 and the light chain variable region of SEQ ID NO: 183;

24) the heavy chain variable region of SEQ ID NO: 155 and the light chain variable region of SEQ ID NO: 184;

25) the heavy chain variable region of SEQ ID NO: 158 and the light chain variable region of SEQ ID NO: 185;

26) the heavy chain variable region of SEQ ID NO: 159 and the light chain variable region of SEQ ID NO: 186;

27) the heavy chain variable region of SEQ ID NO: 160 and the light chain variable region of SEQ ID NO: 187

28) the heavy chain variable region of SEQ ID NO: 256 and the light chain variable region of SEQ ID NO: 258;

29) the heavy chain variable region of SEQ ID NO: 257 and the light chain variable region of SEQ ID NO: 259;

30) the heavy chain variable region of SEQ ID NO: 756 and the light chain variable region of SEQ ID NO: 784;

31) the heavy chain variable region of SEQ ID NO: 757 and the light chain variable region of SEQ ID NO: 785;

32) the heavy chain variable region of SEQ ID NO: 758 and the light chain variable region of SEQ ID NO: 786;

33) the heavy chain variable region of SEQ ID NO: 759 and the light chain variable region of SEQ ID NO: 787;

34) the heavy chain variable region of SEQ ID NO: 760 and the light chain variable region of SEQ ID NO: 788;

35) the heavy chain variable region of SEQ ID NO: 761 and the light chain variable region of SEQ ID NO: 789;

36) the heavy chain variable region of SEQ ID NO: 762 and the light chain variable region of SEQ ID NO: 790;

37) the heavy chain variable region of SEQ ID NO: 763 and the light chain variable region of SEQ ID NO: 791;

38) the heavy chain variable region of SEQ ID NO: 764 and the light chain variable region of SEQ ID NO: 792;

39) the heavy chain variable region of SEQ ID NO: 765 and the light chain variable region of SEQ ID NO: 793;

40) the heavy chain variable region of SEQ ID NO: 766 and the light chain variable region of SEQ ID NO: 794;

41) the heavy chain variable region of SEQ ID NO: 767 and the light chain variable region of SEQ ID NO: 795;

42) the heavy chain variable region of SEQ ID NO: 768 and the light chain variable region of SEQ ID NO: 796;

43) the heavy chain variable region of SEQ ID NO: 769 and the light chain variable region of SEQ ID NO: 797;

44) the heavy chain variable region of SEQ ID NO: 770 and the light chain variable region of SEQ ID NO: 798;

45) the heavy chain variable region of SEQ ID NO: 771 and the light chain variable region of SEQ ID NO: 799;

46) the heavy chain variable region of SEQ ID NO: 772 and the light chain variable region of SEQ ID NO: 800;

47) the heavy chain variable region of SEQ ID NO: 773 and the light chain variable region of SEQ ID NO: 801;

48) the heavy chain variable region of SEQ ID NO: 774 and the light chain variable region of SEQ ID NO: 802;

49) the heavy chain variable region of SEQ ID NO: 775 and the light chain variable region of SEQ ID NO: 803;

50) the heavy chain variable region of SEQ ID NO: 776 and the light chain variable region of SEQ ID NO: 804;

51) the heavy chain variable region of SEQ ID NO: 777 and the light chain variable region of SEQ ID NO: 805;

52) the heavy chain variable region of SEQ ID NO: 778 and the light chain variable region of SEQ ID NO: 806;

53) the heavy chain variable region of SEQ ID NO: 779 and the light chain variable region of SEQ ID NO: 807;

54) the heavy chain variable region of SEQ ID NO: 780 and the light chain variable region of SEQ ID NO: 808;

55) the heavy chain variable region of SEQ ID NO: 781 and the light chain variable region of SEQ ID NO: 809;

56) the heavy chain variable region of SEQ ID NO: 782 and the light chain variable region of SEQ ID NO: 810; or

57) the heavy chain variable region of SEQ ID NO: 783 and the light chain variable region of SEQ ID NO: 811.

175. The method of any one of arrangements 149-174, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

176. The method of any one of arrangements 149-175, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846T.4E11, 847.11D6, 847.20H7, 847.21B11, 849.1D2, 849.2D7, 849.2F12, 849.4B2, 849.4F2, or a binding fragment thereof.

177. The method of any one of arrangements 149-176, wherein the anti-Gal3 antibody or binding fragment thereof is selected from the group consisting of TB001, TB006, 19B5.2E6, 14H10.2C9, 15F10.2D6, 20H5.A3, 23H9.2E4, 2D10.2B2, 7D8.2D8, F846C.1B2, F846C.1F5, F846C.1H12, F846C.2H3, F846TC.14E4, F846TC.16B5, F846TC.7F10, F849C.8D10, 846.4D5, 846T.4E11, 847.11D6, 847.20H7, 847.21B11, 849.1D2, 849.2D7, 849.2F12, 849.4B2, 849.4F2, or a binding fragment thereof.

178. The method of any one of arrangements 149-177, wherein the anti-Gal3 antibody or binding fragment thereof disrupts an interaction between Gal3 and an antibody selected from 846.4D5, 15F10.2D6, F846C.1B2, and F846C.1H12.

179. The method of any one of arrangements 149-178, wherein the anti-Gal3 antibody or binding fragment thereof competes with an antibody that belongs to bins 3, 8, 17 or 24 for binding to Gal3.

180. A method of disrupting an interaction between galectin-3 (Gal3) and a transforming growth factor beta (TGF-b) receptor, the method comprising:

contacting an interaction between Gal3 and the TGF-b receptor with an antibody or binding fragment thereof that selectively binds to Gal3 and disrupts the interaction between Gal3 and the TGF-b receptor.

181. The method of arrangement 180, wherein Gal3 is expressed by a cell.

182. The method of arrangement 180, wherein Gal3 is secreted by a cell.

183. The method of any one of arrangements 180-182, wherein the TGF-b receptor is expressed by a cell.

184. A method of treating fibrosis in a subject in need thereof, the method comprising: administering to the subject an antibody or binding fragment thereof that selectively binds to Gal3 and disrupts an interaction between Gal3 and the TGF-b receptor, thereby treating fibrosis in the subject.

185. The method of arrangement 184, wherein the fibrosis is liver fibrosis, kidney fibrosis, cardiac fibrosis, arterial fibrosis, venous thrombosis, or pulmonary fibrosis.

186. A method of treating non-alcoholic fatty liver disease (NAFLD) or non-alcoholic steatohepatitis (NASH) in a subject in need thereof, the method comprising: administering to the subject an antibody or binding fragment thereof that selectively binds to Gal3 and disrupts an interaction between Gal3 and the TGF-b receptor, thereby treating NAFLD or NASH in the subject.

187. A method of treating an immune-related disorder in a subject in need thereof, the method comprising: administering to the subject an antibody or binding fragment thereof that selectively binds to Gal3 and disrupts an interaction between Gal3 and the TGF-b receptor, thereby treating an immune-related disorder in the subject.

188. The method of arrangement 187, wherein the immune-related disorder is sepsis, atopic dermatitis, or psoriasis.

189. The method of arrangement 187, wherein the immune-related disorder is cancer.

190. The method of arrangement 189, wherein the antibody or binding fragment is administered as a supplement to PD1/PDL1 blockade therapies and/or a CTLA4 blockade therapy.

191. The method of arrangement 190, wherein the PD1/PDL1 blockade therapies comprise pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and/or BMS-986189.

192. The method of arrangement 190, wherein the CTLA4 blockade therapy comprises ipilimumab and/or tremilimumab.

193. The method of any one of arrangements 184-192, wherein the antibody or binding fragment thereof is formulated for systemic administration.

194. The method of any one of arrangements 184-193, wherein the antibody or binding fragment thereof is formulated for parenteral administration.

195. The method of any one of arrangements 184-194, wherein the subject is a mammal.

196. The method of arrangement 195, wherein the mammal is a human.

197. The method of any one of arrangements 180-196, wherein the TGF-b receptor is TGF-b receptor 1, TGF-b receptor 2, or TGF-b receptor 3.

198. A method of disrupting an interaction between Gal3 and a tumor cell surface marker comprising:

contacting the tumor cell surface marker with an anti-Gal3 antibody or binding fragment thereof specific for the N-terminal domain of Gal3, N-terminus of Gal3, or the TRD of Gal3;

wherein the tumor cell surface marker is selected from the group consisting of VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, and FGFR4.

199. The method of arrangement 198, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

200. The method of arrangement 189 or 199, wherein the anti-Gal3 antibody or binding fragment thereof is selected from the group consisting of: 6H6.2D6, 20H5.A3, 20D11.2C6, 4G2.2G6, 13H12.2F8, 19B5.2E6, 15G7.2A7, 23H9.2E4, 19D9.2E5, 2D10.2B2, 4A11.2B5, 14H10.2C9, 3B11.2G2, 13A12.2E5, 7D8.2D8, 15F10.2D6, 23B10.2B12, 12G5.D7, 24D12.2H9, 6B3.2D3, 13G4.2F8, 9H2.2H10, 846.1F5, 846.2H3, 846T.1H2, IMT-001, 4A11.H3L1, 4A11.H1L1, and 4A11.H4L2, or binding fragment thereof.

201. The method of any one of arrangements 189-200, wherein the anti-Gal3 antibody or binding fragment thereof is 2D10.2B2 or 6H6.2D6, or a binding fragment thereof.

202. A method of treating cancer in a subject in need thereof, comprising: administering to the subject an anti-Gal3 antibody or binding fragment thereof specific for the N-terminal domain of Gal3, N-terminus of Gal3, or the TRD of Gal3;

wherein the anti-Gal3 antibody or binding fragment thereof disrupts an interaction between Gal3 and a tumor cell surface marker; and

wherein the tumor cell surface marker is selected from the group consisting of VEGFR1, VEGFR2, VEGFR3, EGFR, PDGFRa, PDGFRb, ErbB2, HGFR (cMet), TNF sRI, CTLA4, CD47, PD-L1, FGFR1 alpha-IIIb, FGFR1 alpha-IIIc, FGFR2 alpha-IIIc, FGFR3 IIIc, and FGFR4.

203. The method of arrangement 202, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

204. The method of arrangement 202 or 203, wherein the anti-Gal3 antibody or binding fragment thereof is selected from the group consisting of: 6H6.2D6, 20H5.A3, 20D11.2C6, 4G2.2G6, 13H12.2F8, 19B5.2E6, 15G7.2A7, 23H9.2E4, 19D9.2E5, 2D10.2B2, 4A11.2B5, 14H10.2C9, 3B11.2G2, 13A12.2E5, 7D8.2D8, 15F10.2D6, 23B10.2B12, 12G5.D7, 24D12.2H9, 6B3.2D3, 13G4.2F8, 9H2.2H10, 846.1F5, 846.2H3, 846T.1H2, IMT-001, 4A11.H3L1, 4A11.H1L1, and 4A11.H4L2, or binding fragment thereof.

205. The method of any one of arrangements 202-204, wherein the anti-Gal3 antibody or binding fragment thereof is 2D10.2B2 or 6H6.2D6, or binding fragment thereof.

206. The method of any one of arrangements 202-205, wherein the cancer is brain cancer, breast cancer, colorectal cancer, kidney cancer, liver cancer, lung cancer, pancreatic cancer, bladder cancer, stomach cancer, or a hematological malignancy.

207. The method of any one of arrangements 202-206, further comprising administering a standard of care treatment, wherein the anti-Gal3 antibody or binding fragment thereof is used as a supplement to the standard of care treatment.

208. The method of arrangement 207, wherein the standard of care treatment comprises surgery, radiation, chemotherapy, targeted therapy, immunotherapy, a PD1/PDL1 blockade therapy, a CTLA4 blockade therapy, temozolomide, or any combination thereof.

209. The method of any one of arrangements 180-208, wherein the antibody or binding fragment thereof binds to an N-terminal domain of Gal3.

210. The method of any one of arrangements 180-209, wherein the antibody or binding fragment thereof binds to an epitope present within a region of Gal3 defined by

| | (a) Peptide 1 |
|---|---|
| SEQ ID NO: 3 | |
| | (ADNFSLHDALSGSGNPNPQG;); |
| | (b) Peptide 4 |
| SEQ ID NO: 6 | |
| | (GAGGYPGASYPGAYPGQAPP;); |
| | (c) Peptide 6 |
| SEQ ID NO: 8 | |
| | (GAYPGQAPPGAYPGAPGAYP;); |
| | (d) Peptide 7 |
| SEQ ID NO: 9 | |
| | (AYPGAPGAYPGAPAPGVYPG;), |

or a combination thereof.

211. The method of any one of arrangements 180-210, wherein the antibody or binding fragment thereof binds to an epitope of Gal3 comprising an amino acid sequence of GxYPG, wherein X is alanine, glycine, or valine.

212. The method of any one of arrangements 180-211, wherein the interaction is reduced to less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, or less than 1%, of an interaction in absence of the antibody or binding fragment thereof.

213. The method of any one of arrangements 180-212, wherein the antibody or binding fragment thereof binds to Gal3 with a dissociation constant (KD) of less than 1 nM, less than 1.2 nM, less than 2 nM, less than 5 nM, less than 10 nM, less than 13.5 nM, less than 15 nM, less than 20 nM, less than 25 nM, or less than 30 nM.

214. The method of any one of arrangements 180-213, wherein the antibody or binding fragment comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

the V_{H}-CDR1 comprises an amino acid sequence having at least 60%, at least 70%, at least 80%, at least 90%, or 100% sequence identity to any amino acid sequence according to SEQ ID NOs: 27-36, 397-399, 588-615;

the V_{H}-CDR2 comprises an amino acid sequence having at least 60%, at least 70%, at least 80%, at least 90%, or 100% sequence identity to any amino acid sequence according to SEQ ID NOs: 45-54, 400-406, 616-643;

the V_{H}-CDR3 comprises an amino acid sequence having at least 60%, at least 70%, at least 80%, at least 90%, or 100% sequence identity to any amino acid sequence according to SEQ ID NOs: 61-69, 71, 408-416, 644-671;

the V_{L}-CDR1 comprises an amino acid sequence having at least 60%, at least 70%, at least 80%, at least 90%, or 100% sequence identity to any amino acid sequence according to SEQ ID NOs: 82-92, 417-426, 672-699;

the V_{L}-CDR2 comprises an amino acid sequence having at least 60%, at least 70%, at least 80%, at least 90%, or 100% sequence identity to any amino acid sequence according to SEQ ID NOs: 102-111, 427-428, 700-727; and

the V_{L}-CDR3 comprises an amino acid sequence having at least 60%, at least 70%, at least 80%, at least 90%, or 100% sequence identity to any amino acid sequence according to SEQ ID NOs: 117-127, 429-434, 728-755.

215. The method of any one of arrangements 180-214, wherein the antibody or binding fragment comprises:

1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161;

2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162;

3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163;

4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164;

5) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171;

6) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165;

7) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166;

8) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167;

9) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168;

10) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169;

11) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170;

12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172;

13) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

14) the V_{H}-CDR1, V_{H}-CDR2, V_{H}-CDR3 of the V_{H}-CDR1, V_{H}-CDR2, V_{H}-CDR3 within SEQ ID NO: 436 and the V_{L}-CDR1, V_{L}-CDR2, V_{L}-CDR3 of the V_{L}-CDR1, V_{L}-CDR2, V_{L}-CDR3 within SEQ ID NO: 451;

15) the V_{H}-CDR1, V_{H}-CDR2, V_{H}-CDR3 of the V_{H}-CDR1, V_{H}-CDR2, V_{H}-CDR3 within SEQ ID NO: 438 and the V_{L}-CDR1, V_{L}-CDR2, V_{L}-CDR3 of the V_{L}-CDR1, V_{L}-CDR2, V_{L}-CDR3 within SEQ ID NO: 453;

16) the V_{H}-CDR1, V_{H}-CDR2, V_{H}-CDR3 of the V_{H}-CDR1, V_{H}-CDR2, V_{H}-CDR3 within SEQ ID NO: 439 and the V_{L}-CDR1, V_{L}-CDR2, V_{L}-CDR3 of the V_{L}-CDR1, V_{L}-CDR2, V_{L}-CDR3 within SEQ ID NO: 162;

17) the V_{H}-CDR1, V_{H}-CDR2, V_{H}-CDR3 of the V_{H}-CDR1, V_{H}-CDR2, V_{H}-CDR3 within SEQ ID NO: 440 and the V_{L}-CDR1, V_{L}-CDR2, V_{L}-CDR3 of the V_{L}-CDR1, V_{L}-CDR2, V_{L}-CDR3 within SEQ ID NO: 454;

18) the V_{H}-CDR1, V_{H}-CDR2, V_{H}-CDR3 of the V_{H}-CDR1, V_{H}-CDR2, V_{H}-CDR3 within SEQ ID NO: 441 and the V_{L}-CDR1, V_{L}-CDR2, V_{L}-CDR3 of the V_{L}-CDR1, V_{L}-CDR2, V_{L}-CDR3 within SEQ ID NO: 455;

19) the V_{H}-CDR1, V_{H}-CDR2, V_{H}-CDR3 of the V_{H}-CDR1, V_{H}-CDR2, V_{H}-CDR3 within SEQ ID NO: 442 and the V_{L}-CDR1, V_{L}-CDR2, V_{L}-CDR3 of the V_{L}-CDR1, V_{L}-CDR2, V_{L}-CDR3 within SEQ ID NO: 456;

20) the V_{H}-CDR1, V_{H}-CDR2, V_{H}-CDR3 of the V_{H}-CDR1, V_{H}-CDR2, V_{H}-CDR3 within SEQ ID NO: 443 and the V_{L}-CDR1, V_{L}-CDR2, V_{L}-CDR3 of the V_{L}-CDR1, V_{L}-CDR2, V_{L}-CDR3 within SEQ ID NO: 457;

21) the V_{H}-CDR1, V_{H}-CDR2, V_{H}-CDR3 of the V_{H}-CDR1, V_{H}-CDR2, V_{H}-CDR3 within SEQ ID NO: 444 and the V_{L}-CDR1, V_{L}-CDR2, V_{L}-CDR3 of the V_{L}-CDR1, V_{L}-CDR2, V_{L}-CDR3 within SEQ ID NO: 458;

22) the V_{H}-CDR1, V_{H}-CDR2, V_{H}-CDR3 of the V_{H}-CDR1, V_{H}-CDR2, V_{H}-CDR3 within SEQ ID NO: 445 and the V_{L}-CDR1, V_{L}-CDR2, V_{L}-CDR3 of the V_{L}-CDR1, V_{L}-CDR2, V_{L}-CDR3 within SEQ ID NO: 459;

23) the V_{H}-CDR1, V_{H}-CDR2, V_{H}-CDR3 of the V_{H}-CDR1, V_{H}-CDR2, V_{H}-CDR3 within SEQ ID NO: 446 and the V_{L}-CDR1, V_{L}-CDR2, V_{L}-CDR3 of the V_{L}-CDR1, V_{L}-CDR2, V_{L}-CDR3 within SEQ ID NO: 460;

24) the V_{H}-CDR1, V_{H}-CDR2, V_{H}-CDR3 of the V_{H}-CDR1, V_{H}-CDR2, V_{H}-CDR3 within SEQ ID NO: 447 and the V_{L}-CDR1, V_{L}-CDR2, V_{L}-CDR3 of the V_{L}-CDR1, V_{L}-CDR2, V_{L}-CDR3 within SEQ ID NO: 461;

25) the V_{H}-CDR1, V_{H}-CDR2, V_{H}-CDR3 of the V_{H}-CDR1, V_{H}-CDR2, V_{H}-CDR3 within SEQ ID NO: 448 and the V_{L}-CDR1, V_{L}-CDR2, V_{L}-CDR3 of the V_{L}-CDR1, V_{L}-CDR2, V_{L}-CDR3 within SEQ ID NO: 462;

26) the V_{H}-CDR1, V_{H}-CDR2, V_{H}-CDR3 of the V_{H}-CDR1, V_{H}-CDR2, V_{H}-CDR3 within SEQ ID NO: 449 and the V_{L}-CDR1, V_{L}-CDR2, V_{L}-CDR3 of the V_{L}-CDR1, V_{L}-CDR2, V_{L}-CDR3 within SEQ ID NO: 463;

27) the V_{H}-CDR1, V_{H}-CDR2, V_{H}-CDR3 of the V_{H}-CDR1, V_{H}-CDR2, V_{H}-CDR3 within SEQ ID NO: 450 and the V_{L}-CDR1, V_{L}-CDR2, V_{L}-CDR3 of the V_{L}-CDR1, V_{L}-CDR2, V_{L}-CDR3 within SEQ ID NO: 464;

28) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784;

29) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785;

30) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786;

31) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787;

32) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788;

33) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789;

34) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790;

35) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791;

36) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792;

37) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793;

38) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794;

39) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795;

40) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796;

41) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797;

42) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798;

43) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799;

44) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800;

45) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801;

46) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802;

47) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803;

48) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804;

49) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805;

50) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806;

51) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807;

52) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808;

53) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809;

54) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or

55) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

216. The method of any one of arrangements 180-215, wherein the antibody or binding fragment comprises:

1) the heavy chain variable region of SEQ ID NO: 136 and the light chain variable region of SEQ ID NO: 161;

2) the heavy chain variable region of SEQ ID NO: 137 and the light chain variable region of SEQ ID NO: 162;

3) the heavy chain variable region of SEQ ID NO: 138 and the light chain variable region of SEQ ID NO: 163;

4) the heavy chain variable region of SEQ ID NO: 139 and the light chain variable region of SEQ ID NO: 164;

5) the heavy chain variable region of SEQ ID NO: 139 and the light chain variable region of SEQ ID NO: 171;

6) the heavy chain variable region of SEQ ID NO: 140 and the light chain variable region of SEQ ID NO: 165;

7) the heavy chain variable region of SEQ ID NO: 141 and the light chain variable region of SEQ ID NO: 166;

8) the heavy chain variable region of SEQ ID NO: 142 and the light chain variable region of SEQ ID NO: 167;

9) the heavy chain variable region of SEQ ID NO: 143 and the light chain variable region of SEQ ID NO: 168;

10) the heavy chain variable region of SEQ ID NO: 144 and the light chain variable region of SEQ ID NO: 169;

11) the heavy chain variable region of SEQ ID NO: 145 and the light chain variable region of SEQ ID NO: 170;

12) the heavy chain variable region of SEQ ID NO: 146 and the light chain variable region of SEQ ID NO: 172;

13) the heavy chain variable region of SEQ ID NO: 148 and the light chain variable region of SEQ ID NO: 174;

14) the heavy chain variable region of SEQ ID NO: 436 and the light chain variable region of SEQ ID NO: 451;

15) the heavy chain variable region of SEQ ID NO: 438 and the light chain variable region of SEQ ID NO: 453;

16) the heavy chain variable region of SEQ ID NO: 439 and the light chain variable region of SEQ ID NO: 162;

17) the heavy chain variable region of SEQ ID NO: 440 and the light chain variable region of SEQ ID NO: 454;

18) the heavy chain variable region of SEQ ID NO: 441 and the light chain variable region of SEQ ID NO: 455;

19) the heavy chain variable region of SEQ ID NO: 442 and the light chain variable region of SEQ ID NO: 456;

20) the heavy chain variable region of SEQ ID NO: 443 and the light chain variable region of SEQ ID NO: 457;

21) the heavy chain variable region of SEQ ID NO: 444 and the light chain variable region of SEQ ID NO: 458;

22) the heavy chain variable region of SEQ ID NO: 445 and the light chain variable region of SEQ ID NO: 459;

23) the heavy chain variable region of SEQ ID NO: 446 and the light chain variable region of SEQ ID NO: 460;

24) the heavy chain variable region of SEQ ID NO: 447 and the light chain variable region of SEQ ID NO: 461;

25) the heavy chain variable region of SEQ ID NO: 448 and the light chain variable region of SEQ ID NO: 462;

26) the heavy chain variable region of SEQ ID NO: 449 and the light chain variable region of SEQ ID NO: 463;

27) the heavy chain variable region of SEQ ID NO: 450 and the light chain variable region of SEQ ID NO: 464;

28) the heavy chain variable region of SEQ ID NO: 756 and the light chain variable region of SEQ ID NO: 784;

29) the heavy chain variable region of SEQ ID NO: 757 and the light chain variable region of SEQ ID NO: 785;

30) the heavy chain variable region of SEQ ID NO: 758 and the light chain variable region of SEQ ID NO: 786;

31) the heavy chain variable region of SEQ ID NO: 759 and the light chain variable region of SEQ ID NO: 787;

32) the heavy chain variable region of SEQ ID NO: 760 and the light chain variable region of SEQ ID NO: 788;

33) the heavy chain variable region of SEQ ID NO: 761 and the light chain variable region of SEQ ID NO: 789;

34) the heavy chain variable region of SEQ ID NO: 762 and the light chain variable region of SEQ ID NO: 790;

35) the heavy chain variable region of SEQ ID NO: 763 and the light chain variable region of SEQ ID NO: 791;

36) the heavy chain variable region of SEQ ID NO: 764 and the light chain variable region of SEQ ID NO: 792;

37) the heavy chain variable region of SEQ ID NO: 765 and the light chain variable region of SEQ ID NO: 793;

38) the heavy chain variable region of SEQ ID NO: 766 and the light chain variable region of SEQ ID NO: 794;

39) the heavy chain variable region of SEQ ID NO: 767 and the light chain variable region of SEQ ID NO: 795;

40) the heavy chain variable region of SEQ ID NO: 768 and the light chain variable region of SEQ ID NO: 796;

41) the heavy chain variable region of SEQ ID NO: 769 and the light chain variable region of SEQ ID NO: 797;

42) the heavy chain variable region of SEQ ID NO: 770 and the light chain variable region of SEQ ID NO: 798;

43) the heavy chain variable region of SEQ ID NO: 771 and the light chain variable region of SEQ ID NO: 799;

44) the heavy chain variable region of SEQ ID NO: 772 and the light chain variable region of SEQ ID NO: 800;

45) the heavy chain variable region of SEQ ID NO: 773 and the light chain variable region of SEQ ID NO: 801;

46) the heavy chain variable region of SEQ ID NO: 774 and the light chain variable region of SEQ ID NO: 802;

47) the heavy chain variable region of SEQ ID NO: 775 and the light chain variable region of SEQ ID NO: 803;

48) the heavy chain variable region of SEQ ID NO: 776 and the light chain variable region of SEQ ID NO: 804;

49) the heavy chain variable region of SEQ ID NO: 777 and the light chain variable region of SEQ ID NO: 805;

50) the heavy chain variable region of SEQ ID NO: 778 and the light chain variable region of SEQ ID NO: 806;

51) the heavy chain variable region of SEQ ID NO: 779 and the light chain variable region of SEQ ID NO: 807;

52) the heavy chain variable region of SEQ ID NO: 780 and the light chain variable region of SEQ ID NO: 808;

53) the heavy chain variable region of SEQ ID NO: 781 and the light chain variable region of SEQ ID NO: 809;

54) the heavy chain variable region of SEQ ID NO: 782 and the light chain variable region of SEQ ID NO: 810; or

55) the heavy chain variable region of SEQ ID NO: 783 and the light chain variable region of SEQ ID NO: 811.

217. The method of any one of arrangements 180-216, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

218. The method of any one of arrangements 180-217, wherein the antibody or binding fragment is selected from the group consisting of: 13H12.2F8, 19D9.2E5, 14H10.2C9, 2D10.2B2, 4A11.2B5, 6H6.2D6, 20H5.A3, 19B5.2E6, 23H9.2E4, 20D11.2C6, 15G7.2A7, 4G2.2G6, 3B11.2G2, 13A12.2E5, 7D8.2D8, 15F10.2D6, 12G5.D7, 24D12.2H9, 13G4.2F8, 9H2.2H10, 23B10.2B12, 6B3.2D3, 846.1F5, 846.2H3, 846T.1H2, IMT-001, 4A11.H3L1, 4A11.H1L1 and 4A11.H4L2, or binding fragment thereof.

219. The method of any one of arrangements 180-218, wherein the antibody or binding fragment thereof comprises a humanized antibody or binding fragment.

220. The method of any one of arrangements 180-219, wherein the antibody or binding fragment thereof comprises a full-length antibody or a binding fragment thereof.

221. The method of any one of arrangements 180-220, wherein the antibody or binding fragment thereof comprises a bispecific antibody or a binding fragment thereof.

222. The method of any one of arrangements 180-221, wherein the antibody or binding fragment thereof comprises a monovalent Fab′, a divalent Fab2, a single-chain variable fragment (scFv), a diabody, a minibody, a nanobody, a single-domain antibody (sdAb), or a camelid antibody, or binding fragment thereof.

223. The method of any one of arrangements 180-222, wherein the antibody or binding fragment thereof comprises an IgG framework.

224. The method of any one of arrangements 180-223, wherein the antibody or binding fragment thereof comprises an IgG1, IgG2, or IgG4 framework.

225. An anti-Gal3 antibody or binding fragment thereof comprising (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

the V_{H}-CDR1 comprises an amino acid sequence having at least 60%, at least 70%, at least 80%, at least 90%, or 100% sequence identity to any amino acid sequence according to SEQ ID NOs: 27-36, 397-399,

the V_{H}-CDR2 comprises an amino acid sequence having at least 60%, at least 70%, at least 80%, at least 90%, or 100% sequence identity to any amino acid sequence according to SEQ ID NOs: 45-54, 400-406,

the V_{H}-CDR3 comprises an amino acid sequence having at least 60%, at least 70%, at least 80%, at least 90%, or 100% sequence identity to any amino acid sequence according to SEQ ID NOs: 61-69, 71, 408-416 the V_{L}-CDR1 comprises an amino acid sequence having at least 60%, at least 70%, at least 80%, at least 90%, or 100% sequence identity to any amino acid sequence according to SEQ ID NOs: 82-92, 417-426,

the V_{L}-CDR2 comprises an amino acid sequence having at least 60%, at least 70%, at least 80%, at least 90%, or 100% sequence identity to any amino acid sequence according to SEQ ID NOs: 102-111, 427-428, and

the V_{L}-CDR3 comprises an amino acid sequence having at least 60%, at least 70%, at least 80%, at least 90%, or 100% sequence identity to any amino acid sequence according to SEQ ID NOs: 117-127, 429-434.

226. The anti-Gal3 antibody or binding fragment thereof of arrangement 225, wherein the anti-Gal3 antibody or binding fragment thereof comprises:

a) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161;

b) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162;

c) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163;

d) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164;

e) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171;

f) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165;

g) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166;

h) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167;

i) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168;

j) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169;

k) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170;

1) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172;

m) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

n) the V_{H}-CDR1, V_{H}-CDR2, V_{H}-CDR3 of the V_{H}-CDR1, V_{H}-CDR2, V_{H}-CDR3 within SEQ ID NO: 436 and the V_{L}-CDR1, V_{L}-CDR2, V_{L}-CDR3 of the V_{L}-CDR1, V_{L}-CDR2, V_{L}-CDR3 within SEQ ID NO: 451;

o) the V_{H}-CDR1, V_{H}-CDR2, V_{H}-CDR3 of the V_{H}-CDR1, V_{H}-CDR2, V_{H}-CDR3 within SEQ ID NO: 438 and the V_{L}-CDR1, V_{L}-CDR2, V_{L}-CDR3 of the V_{L}-CDR1, V_{L}-CDR2, V_{L}-CDR3 within SEQ ID NO: 453;

p) the V_{H}-CDR1, V_{H}-CDR2, V_{H}-CDR3 of the V_{H}-CDR1, V_{H}-CDR2, V_{H}-CDR3 within SEQ ID NO: 439 and the V_{L}-CDR1, V_{L}-CDR2, V_{L}-CDR3 of the V_{L}-CDR1, V_{L}-CDR2, V_{L}-CDR3 within SEQ ID NO: 162;

q) the V_{H}-CDR1, V_{H}-CDR2, V_{H}-CDR3 of the V_{H}-CDR1, V_{H}-CDR2, V_{H}-CDR3 within SEQ ID NO: 440 and the V_{L}-CDR1, V_{L}-CDR2, V_{L}-CDR3 of the V_{L}-CDR1, V_{L}-CDR2, V_{L}-CDR3 within SEQ ID NO: 454;

r) the V_{H}-CDR1, V_{H}-CDR2, V_{H}-CDR3 of the V_{H}-CDR1, V_{H}-CDR2, V_{H}-CDR3 within SEQ ID NO: 441 and the V_{L}-CDR1, V_{L}-CDR2, V_{L}-CDR3 of the V_{L}-CDR1, V_{L}-CDR2, V_{L}-CDR3 within SEQ ID NO: 455;

s) the V_{H}-CDR1, V_{H}-CDR2, V_{H}-CDR3 of the V_{H}-CDR1, V_{H}-CDR2, V_{H}-CDR3 within SEQ ID NO: 442 and the V_{L}-CDR1, V_{L}-CDR2, V_{L}-CDR3 of the V_{L}-CDR1, V_{L}-CDR2, V_{L}-CDR3 within SEQ ID NO: 456;

t) the V_{H}-CDR1, V_{H}-CDR2, V_{H}-CDR3 of the V_{H}-CDR1, V_{H}-CDR2, V_{H}-CDR3 within SEQ ID NO: 443 and the V_{L}-CDR1, V_{L}-CDR2, V_{L}-CDR3 of the V_{L}-CDR1, V_{L}-CDR2, V_{L}-CDR3 within SEQ ID NO: 457;

u) the V_{H}-CDR1, V_{H}-CDR2, V_{H}-CDR3 of the V_{H}-CDR1, V_{H}-CDR2, V_{H}-CDR3 within SEQ ID NO: 444 and the V_{L}-CDR1, V_{L}-CDR2, V_{L}-CDR3 of the V_{L}-CDR1, V_{L}-CDR2, V_{L}-CDR3 within SEQ ID NO: 458;

v) the V_{H}-CDR1, V_{H}-CDR2, V_{H}-CDR3 of the V_{H}-CDR1, V_{H}-CDR2, V_{H}-CDR3 within SEQ ID NO: 445 and the V_{L}-CDR1, V_{L}-CDR2, V_{L}-CDR3 of the V_{L}-CDR1, V_{L}-CDR2, V_{L}-CDR3 within SEQ ID NO: 459;

w) the V_{H}-CDR1, V_{H}-CDR2, V_{H}-CDR3 of the V_{H}-CDR1, V_{H}-CDR2, V_{H}-CDR3 within SEQ ID NO: 446 and the V_{L}-CDR1, V_{L}-CDR2, V_{L}-CDR3 of the V_{L}-CDR1, V_{L}-CDR2, V_{L}-CDR3 within SEQ ID NO: 460;

x) the V_{H}-CDR1, V_{H}-CDR2, V_{H}-CDR3 of the V_{H}-CDR1, V_{H}-CDR2, V_{H}-CDR3 within SEQ ID NO: 447 and the V_{L}-CDR1, V_{L}-CDR2, V_{L}-CDR3 of the V_{L}-CDR1, V_{L}-CDR2, V_{L}-CDR3 within SEQ ID NO: 461;

y) the V_{H}-CDR1, V_{H}-CDR2, V_{H}-CDR3 of the V_{H}-CDR1, V_{H}-CDR2, V_{H}-CDR3 within SEQ ID NO: 448 and the V_{L}-CDR1, V_{L}-CDR2, V_{L}-CDR3 of the V_{L}-CDR1, V_{L}-CDR2, V_{L}-CDR3 within SEQ ID NO: 462;

z) the V_{H}-CDR1, V_{H}-CDR2, V_{H}-CDR3 of the V_{H}-CDR1, V_{H}-CDR2, V_{H}-CDR3 within SEQ ID NO: 449 and the V_{L}-CDR1, V_{L}-CDR2, V_{L}-CDR3 of the V_{L}-CDR1, V_{L}-CDR2, V_{L}-CDR3 within SEQ ID NO: 463; or

aa) the V_{H}-CDR1, V_{H}-CDR2, V_{H}-CDR3 of the V_{H}-CDR1, V_{H}-CDR2, V_{H}-CDR3 within SEQ ID NO: 450 and the V_{L}-CDR1, V_{L}-CDR2, V_{L}-CDR3 of the V_{L}-CDR1, V_{L}-CDR2, V_{L}-CDR3 within SEQ ID NO: 464.

227. The anti-Gal3 antibody or binding fragment thereof of arrangement 225 or 226, wherein the antibody or binding fragment comprises:

a) the heavy chain variable region of SEQ ID NO: 136 and the light chain variable region of SEQ ID NO: 161;

b) the heavy chain variable region of SEQ ID NO: 137 and the light chain variable region of SEQ ID NO: 162;

c) the heavy chain variable region of SEQ ID NO: 138 and the light chain variable region of SEQ ID NO: 163;

d) the heavy chain variable region of SEQ ID NO: 139 and the light chain variable region of SEQ ID NO: 164;

e) the heavy chain variable region of SEQ ID NO: 139 and the light chain variable region of SEQ ID NO: 171;

f) the heavy chain variable region of SEQ ID NO: 140 and the light chain variable region of SEQ ID NO: 165;

g) the heavy chain variable region of SEQ ID NO: 141 and the light chain variable region of SEQ ID NO: 166;

h) the heavy chain variable region of SEQ ID NO: 142 and the light chain variable region of SEQ ID NO: 167;

i) the heavy chain variable region of SEQ ID NO: 143 and the light chain variable region of SEQ ID NO: 168;

j) the heavy chain variable region of SEQ ID NO: 144 and the light chain variable region of SEQ ID NO: 169;

k) the heavy chain variable region of SEQ ID NO: 145 and the light chain variable region of SEQ ID NO: 170;

1) the heavy chain variable region of SEQ ID NO: 146 and the light chain variable region of SEQ ID NO: 172;

m) the heavy chain variable region of SEQ ID NO: 148 and the light chain variable region of SEQ ID NO: 174;

n) the heavy chain variable region of SEQ ID NO: 436 and the light chain variable region of SEQ ID NO: 451;

o) the heavy chain variable region of SEQ ID NO: 438 and the light chain variable region of SEQ ID NO: 453;

p) the heavy chain variable region of SEQ ID NO: 439 and the light chain variable region of SEQ ID NO: 162;

q) the heavy chain variable region of SEQ ID NO: 440 and the light chain variable region of SEQ ID NO: 454;

r) the heavy chain variable region of SEQ ID NO: 441 and the light chain variable region of SEQ ID NO: 455;

s) the heavy chain variable region of SEQ ID NO: 442 and the light chain variable region of SEQ ID NO: 456;

t) the heavy chain variable region of SEQ ID NO: 443 and the light chain variable region of SEQ ID NO: 457;

u) the heavy chain variable region of SEQ ID NO: 444 and the light chain variable region of SEQ ID NO: 458;

v) the heavy chain variable region of SEQ ID NO: 445 and the light chain variable region of SEQ ID NO: 459;

w) the heavy chain variable region of SEQ ID NO: 446 and the light chain variable region of SEQ ID NO: 460;

x) the heavy chain variable region of SEQ ID NO: 447 and the light chain variable region of SEQ ID NO: 461;

y) the heavy chain variable region of SEQ ID NO: 448 and the light chain variable region of SEQ ID NO: 462;

z) the heavy chain variable region of SEQ ID NO: 449 and the light chain variable region of SEQ ID NO: 463; or aa) the heavy chain variable region of SEQ ID NO: 450 and the light chain variable region of SEQ ID NO: 464.

228. The anti-Gal3 antibody or binding fragment thereof of any one of arrangements 225-227, wherein the anti-Gal3 antibody or binding fragment thereof is selected from the group consisting of: 6H6.2D6, 20H5.A3, 20D11.2C6, 4G2.2G6, 13H12.2F8, 19B5.2E6, 15G7.2A7, 23H9.2E4, 19D9.2E5, 2D10.2B2, 4A11.2B5, 14H10.2C9, 3B11.2G2, 13A12.2E5, 7D8.2D8, 15F10.2D6, 23B10.2B12, 12G5.D7, 24D12.2H9, 6B3.2D3, 13G4.2F8, 9H2.2H10, 846.1F5, 846.2H3, 846T.1H2, IMT-001, 4A11.H3L1, 4A11.H1L1, and 4A11.H4L2 or binding fragment thereof.

229. The anti-Gal3 antibody or binding fragment thereof of any one of arrangements 225-228, wherein the antibody or binding fragment thereof does not bind to the C-terminus of Gal3.

230. The anti-Gal3 antibody or binding fragment thereof of any one of arrangements 225-229, wherein the antibody or binding fragment thereof does not bind to the C-terminal domain of Gal3.

231. The anti-Gal3 antibody or binding fragment thereof of any one of arrangements 225-230, wherein the antibody or binding fragment thereof does not bind to the C-terminal carbohydrate-recognition-binding domain.

232. The anti-Gal3 antibody or binding fragment thereof of any one of arrangements 225-231, wherein the antibody or binding fragment thereof does not bind to amino acids 112-250 of Gal3 or a subregion thereof.

233. The anti-Gal3 antibody or binding fragment thereof of any one of arrangements 225-232, wherein the antibody or binding fragment thereof binds to the N-terminus of Gal3.

234. The anti-Gal3 antibody or binding fragment thereof of any one of arrangements 225-233, wherein the antibody or binding fragment thereof binds to the N-terminal domain of Gal3.

235. The anti-Gal3 antibody or binding fragment thereof of any one of arrangements 225-234, wherein the antibody or binding fragment thereof binds to amino acids 1-111 of Gal3 or a subregion thereof.

236. The anti-Gal3 antibody or binding fragment thereof of any one of arrangements 225-235, wherein the antibody or binding fragment thereof binds to the tandem repeat domain of Gal3.

237. The anti-Gal3 antibody or binding fragment thereof of any one of arrangements 225-236, wherein the antibody or binding fragment thereof binds to Peptide 1 (ADNFSLHDALSGSGNPNPQG; SEQ ID NO: 3).

238. The anti-Gal3 antibody or binding fragment thereof of any one of arrangements 225-237, wherein the antibody or binding fragment thereof binds to Peptide 6 (GAYPGQAPPGAYPGAPGAYP; SEQ ID NO: 8).

239. The anti-Gal3 antibody or binding fragment thereof of any one of arrangements 225-238, wherein the antibody or binding fragment thereof binds to Peptide 7 (AYPGAPGAYPGAPAPGVYPG; SEQ ID NO: 9).

240. The anti-Gal3 antibody or binding fragment thereof of any one of arrangements 225-239, wherein the antibody or binding fragment thereof is 2D10.2B2 or 6H6.2D6, or binding fragment thereof.

241. The anti-Gal3 antibody or binding fragment thereof of any one of arrangements 225-240, wherein the antibody or binding fragment thereof is selected from the group consisting of 2D10.2B2 or 6H6.2D6, or binding fragment thereof.

242. The anti-Gal3 antibody or binding fragment thereof of any one of arrangements 225-241, wherein the antibody or binding fragment thereof inhibits tumor cell growth in vitro.

243. The anti-Gal3 antibody or binding fragment thereof of any one of arrangements 225-242, wherein the antibody or binding fragment thereof retards brain tumor growth.

244. A pharmaceutical composition comprising the anti-Gal3 antibody or binding fragment thereof of any one of arrangements 225-243 and at least one pharmaceutically acceptable carrier, excipient, diluent, or adjuvant.

245. An antibody that binds to human Gal3 and competes with an anti-Gal3 antibody or binding fragment thereof for binding to human Gal3, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

246. An antibody that binds to human Gal3 and competes with an anti-Gal3 antibody or binding fragment thereof for binding to human Gal3, wherein the anti-Gal3 antibody or binding fragment thereof is selected from the group consisting of: 6H6.2D6, 20H5.A3, 20D11.2C6, 4G2.2G6, 13H12.2F8, 19B5.2E6, 15G7.2A7, 23H9.2E4, 19D9.2E5, 2D10.2B2, 4A11.2B5, 14H10.2C9, 3B11.2G2, 13A12.2E5, 7D8.2D8, 15F10.2D6, 23B10.2B12, 12G5.D7, 24D12.2H9, 6B3.2D3, 13G4.2F8, 9H2.2H10, 846.1F5, 846.2H3, 846T.1H2, IMT001, 4A11.H3L1, 4A11.H1L1, and 4A11.H4L2 or binding fragment thereof.

247. A method for identifying an antibody or binding fragment capable of disrupting an interaction between Gal3 and a TGF-b receptor, the method comprising:

(a) contacting Gal3 protein with an antibody or binding fragment that selectively binds to Gal3, thereby forming a Gal3-antibody complex;

(b) contacting the Gal3-antibody complex with the TGF-b receptor protein;

(c) removing unbound TGF-b receptor protein; and

(d) detecting TGF-b receptor protein bound to the Gal3-antibody complex;

wherein the antibody or binding fragment is capable of disrupting an interaction of Gal3 and the TGF-b receptor when the TGF-b receptor protein is not detected in (d).

248. The method of arrangement 247, wherein the method comprises an immunoassay.

249. The method of arrangement 248, wherein the immunoassay is an enzyme-linked immunosorbent assay.

250. The method of any one of arrangements 247-249, wherein the TGF-b receptor is TGF-b receptor 1, TGF-b receptor 2, or TGF-b receptor 3.

251. Use of an anti-Gal3 antibody or binding fragment in the manufacture of a medicament or composition for the treatment of fibrosis, liver fibrosis, non-alcoholic fatty liver disease (NAFLD), non-alcoholic steatohepatitis (NASH), kidney fibrosis, cardiac fibrosis, arterial fibrosis, venous thrombosis, or pulmonary fibrosis.

252. Use of an anti-Gal3 antibody or binding fragment in the manufacture of a medicament or composition for the treatment of an immune-related disorder.

253. The use of arrangement 252, wherein the immune-related disorder is sepsis, atopic dermatitis, or psoriasis.

254. The use of arrangement 252, wherein the immune-related disorder is cancer.

255. The use of arrangement 254, wherein the medicament is used as a supplement to PD1/PDL1 blockade therapies or CTLA4 blockade therapies.

256. The use of arrangement 255, wherein the PD1/PDL1 blockade therapies comprise pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, and/or BMS-986189.

257. The use of arrangement 255, wherein the CTLA4 blockade therapy comprises ipilimumab and/or tremilimumab.

258. Use of an anti-Gal3 antibody or binding fragment thereof for the treatment of fibrosis, liver fibrosis, NAFLD, NASH, kidney fibrosis, cardiac fibrosis, arterial fibrosis, venous thrombosis, or pulmonary fibrosis.

259. Use of an anti-Gal3 antibody or binding fragment thereof for the treatment of cancer.

260. The use of arrangement 259, wherein the cancer is brain cancer, breast cancer, colorectal cancer, kidney cancer, liver cancer, lung cancer, pancreatic cancer, bladder cancer, stomach cancer, or a hematological malignancy.

261. Use of an anti-Gal3 antibody or binding fragment thereof for the inhibition of tumor cell growth in vitro.

262. Use of an anti-Gal3 antibody or binding fragment thereof for the retardation of brain tumor growth.

263. The use of any one of arrangements 252-262, wherein the anti-Gal3 antibody or binding fragment thereof comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein

the V_{H}-CDR1 comprises an amino acid sequence having at least 60%, at least 70%, at least 80%, at least 90%, or 100% sequence identity to any amino acid sequence according to SEQ ID NOs: 27-36, 397-399, 588-615,

the V_{H}-CDR2 comprises an amino acid sequence having at least 60%, at least 70%, at least 80%, at least 90%, or 100% sequence identity to any amino acid sequence according to SEQ ID NOs: 45-54, 400-406, 616-643,

the V_{H}-CDR3 comprises an amino acid sequence having at least 60%, at least 70%, at least 80%, at least 90%, or 100% sequence identity to any amino acid sequence according to SEQ ID NOs: 61-69, 71, 408-416, 644-671,

the V_{L}-CDR1 comprises an amino acid sequence having at least 60%, at least 70%, at least 80%, at least 90%, or 100% sequence identity to any amino acid sequence according to SEQ ID NOs: 82-92, 417-426, 672-699,

the V_{L}-CDR2 comprises an amino acid sequence having at least 60%, at least 70%, at least 80%, at least 90%, or 100% sequence identity to any amino acid sequence according to SEQ ID NOs: 102-111, 427-428, 700-727, and

the V_{L}-CDR3 comprises an amino acid sequence having at least 60%, at least 70%, at least 80%, at least 90%, or 100% sequence identity to any amino acid sequence according to SEQ ID NOs: 117-127, 429-434, 728-755.

264. The use of any one of arrangements 252-263, wherein the anti-Gal3 antibody or binding fragment comprises:

1) the V_{H}-CDR1, V_{H}-CDR2, V_{H}-CDR3 of the V_{H}-CDR1, V_{H}-CDR2, V_{H}-CDR3 within SEQ ID NO: 136 and the V_{L}-CDR1, V_{L}-CDR2, V_{L}-CDR3 of the V_{L}-CDR1, V_{L}-CDR2, V_{L}-CDR3 within SEQ ID NO: 161;

2) the V_{H}-CDR1, V_{H}-CDR2, V_{H}-CDR3 of the V_{H}-CDR1, V_{H}-CDR2, V_{H}-CDR3 within SEQ ID NO: 137 and the V_{L}-CDR1, V_{L}-CDR2, V_{L}-CDR3 of the V_{L}-CDR1, V_{L}-CDR2, V_{L}-CDR3 within SEQ ID NO: 162;

3) the V_{H}-CDR1, V_{H}-CDR2, V_{H}-CDR3 of the V_{H}-CDR1, V_{H}-CDR2, V_{H}-CDR3 within SEQ ID NO: 138 and the V_{L}-CDR1, V_{L}-CDR2, V_{L}-CDR3 of the V_{L}-CDR1, V_{L}-CDR2, V_{L}-CDR3 within SEQ ID NO: 163;

4) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 164;

5) the V_{H}-CDR1, V_{H}-CDR2, V_{H}-CDR3 of the V_{H}-CDR1, V_{H}-CDR2, V_{H}-CDR3 within SEQ ID NO: 139 and the V_{L}-CDR1, V_{L}-CDR2, V_{L}-CDR3 of the V_{L}-CDR1, V_{L}-CDR2, V_{L}-CDR3 within SEQ ID NO: 171;

6) the V_{H}-CDR1, V_{H}-CDR2, V_{H}-CDR3 of the V_{H}-CDR1, V_{H}-CDR2, V_{H}-CDR3 within SEQ ID NO: 140 and the V_{L}-CDR1, V_{L}-CDR2, V_{L}-CDR3 of the V_{L}-CDR1, V_{L}-CDR2, V_{L}-CDR3 within SEQ ID NO: 165;

7) the V_{H}-CDR1, V_{H}-CDR2, V_{H}-CDR3 of the V_{H}-CDR1, V_{H}-CDR2, V_{H}-CDR3 within SEQ ID NO: 141 and the V_{L}-CDR1, V_{L}-CDR2, V_{L}-CDR3 of the V_{L}-CDR1, V_{L}-CDR2, V_{L}-CDR3 within SEQ ID NO: 166;

8) the V_{H}-CDR1, V_{H}-CDR2, V_{H}-CDR3 of the V_{H}-CDR1, V_{H}-CDR2, V_{H}-CDR3 within SEQ ID NO: 142 and the V_{L}-CDR1, V_{L}-CDR2, V_{L}-CDR3 of the V_{L}-CDR1, V_{L}-CDR2, V_{L}-CDR3 within SEQ ID NO: 167;

9) the V_{H}-CDR1, V_{H}-CDR2, V_{H}-CDR3 of the V_{H}-CDR1, V_{H}-CDR2, V_{H}-CDR3 within SEQ ID NO: 143 and the V_{L}-CDR1, V_{L}-CDR2, V_{L}-CDR3 of the V_{L}-CDR1, V_{L}-CDR2, V_{L}-CDR3 within SEQ ID NO: 168;

10) the V_{H}-CDR1, V_{H}-CDR2, V_{H}-CDR3 of the V_{H}-CDR1, V_{H}-CDR2, V_{H}-CDR3 within SEQ ID NO: 144 and the V_{L}-CDR1, V_{L}-CDR2, V_{L}-CDR3 of the V_{L}-CDR1, V_{L}-CDR2, V_{L}-CDR3 within SEQ ID NO: 169;

11) the V_{H}-CDR1, V_{H}-CDR2, V_{H}-CDR3 of the V_{H}-CDR1, V_{H}-CDR2, V_{H}-CDR3 within SEQ ID NO: 145 and the V_{L}-CDR1, V_{L}-CDR2, V_{L}-CDR3 of the V_{L}-CDR1, V_{L}-CDR2, V_{L}-CDR3 within SEQ ID NO: 170;

12) the V_{H}-CDR1, V_{H}-CDR2, V_{H}-CDR3 of the V_{H}-CDR1, V_{H}-CDR2, V_{H}-CDR3 within SEQ ID NO: 146 and the V_{L}-CDR1, V_{L}-CDR2, V_{L}-CDR3 of the V_{L}-CDR1, V_{L}-CDR2, V_{L}-CDR3 within SEQ ID NO: 172;

13) the V_{H}-CDR1, V_{H}-CDR2, V_{H}-CDR3 of the V_{H}-CDR1, V_{H}-CDR2, V_{H}-CDR3 within SEQ ID NO: 148 and the V_{L}-CDR1, V_{L}-CDR2, V_{L}-CDR3 of the V_{L}-CDR1, V_{L}-CDR2, V_{L}-CDR3 within SEQ ID NO: 174;

14) the V_{H}-CDR1, V_{H}-CDR2, V_{H}-CDR3 of the V_{H}-CDR1, V_{H}-CDR2, V_{H}-CDR3 within SEQ ID NO: 436 and the V_{L}-CDR1, V_{L}-CDR2, V_{L}-CDR3 of the V_{L}-CDR1, V_{L}-CDR2, V_{L}-CDR3 within SEQ ID NO: 451;

15) the V_{H}-CDR1, V_{H}-CDR2, V_{H}-CDR3 of the V_{H}-CDR1, V_{H}-CDR2, V_{H}-CDR3 within SEQ ID NO: 438 and the V_{L}-CDR1, V_{L}-CDR2, V_{L}-CDR3 of the V_{L}-CDR1, V_{L}-CDR2, V_{L}-CDR3 within SEQ ID NO: 453;

16) the V_{H}-CDR1, V_{H}-CDR2, V_{H}-CDR3 of the V_{H}-CDR1, V_{H}-CDR2, V_{H}-CDR3 within SEQ ID NO: 439 and the V_{L}-CDR1, V_{L}-CDR2, V_{L}-CDR3 of the V_{L}-CDR1, V_{L}-CDR2, V_{L}-CDR3 within SEQ ID NO: 162;

17) the V_{H}-CDR1, V_{H}-CDR2, V_{H}-CDR3 of the V_{H}-CDR1, V_{H}-CDR2, V_{H}-CDR3 within SEQ ID NO: 440 and the V_{L}-CDR1, V_{L}-CDR2, V_{L}-CDR3 of the V_{L}-CDR1, V_{L}-CDR2, V_{L}-CDR3 within SEQ ID NO: 454;

18) the V_{H}-CDR1, V_{H}-CDR2, V_{H}-CDR3 of the V_{H}-CDR1, V_{H}-CDR2, V_{H}-CDR3 within SEQ ID NO: 441 and the V_{L}-CDR1, V_{L}-CDR2, V_{L}-CDR3 of the V_{L}-CDR1, V_{L}-CDR2, V_{L}-CDR3 within SEQ ID NO: 455;

19) the V_{H}-CDR1, V_{H}-CDR2, V_{H}-CDR3 of the V_{H}-CDR1, V_{H}-CDR2, V_{H}-CDR3 within SEQ ID NO: 442 and the V_{L}-CDR1, V_{L}-CDR2, V_{L}-CDR3 of the V_{L}-CDR1, V_{L}-CDR2, V_{L}-CDR3 within SEQ ID NO: 456;

20) the V_{H}-CDR1, V_{H}-CDR2, V_{H}-CDR3 of the V_{H}-CDR1, V_{H}-CDR2, V_{H}-CDR3 within SEQ ID NO: 443 and the V_{L}-CDR1, V_{L}-CDR2, V_{L}-CDR3 of the V_{L}-CDR1, V_{L}-CDR2, V_{L}-CDR3 within SEQ ID NO: 457;

21) the V_{H}-CDR1, V_{H}-CDR2, V_{H}-CDR3 of the V_{H}-CDR1, V_{H}-CDR2, V_{H}-CDR3 within SEQ ID NO: 444 and the V_{L}-CDR1, V_{L}-CDR2, V_{L}-CDR3 of the V_{L}-CDR1, V_{L}-CDR2, V_{L}-CDR3 within SEQ ID NO: 458;

22) the V_{H}-CDR1, V_{H}-CDR2, V_{H}-CDR3 of the V_{H}-CDR1, V_{H}-CDR2, V_{H}-CDR3 within SEQ ID NO: 445 and the V_{L}-CDR1, V_{L}-CDR2, V_{L}-CDR3 of the V_{L}-CDR1, V_{L}-CDR2, V_{L}-CDR3 within SEQ ID NO: 459;

23) the V_{H}-CDR1, V_{H}-CDR2, V_{H}-CDR3 of the V_{H}-CDR1, V_{H}-CDR2, V_{H}-CDR3 within SEQ ID NO: 446 and the V_{L}-CDR1, V_{L}-CDR2, V_{L}-CDR3 of the V_{L}-CDR1, V_{L}-CDR2, V_{L}-CDR3 within SEQ ID NO: 460;

24) the V_{H}-CDR1, V_{H}-CDR2, V_{H}-CDR3 of the V_{H}-CDR1, V_{H}-CDR2, V_{H}-CDR3 within SEQ ID NO: 447 and the V_{L}-CDR1, V_{L}-CDR2, V_{L}-CDR3 of the V_{L}-CDR1, V_{L}-CDR2, V_{L}-CDR3 within SEQ ID NO: 461;

25) the V_{H}-CDR1, V_{H}-CDR2, V_{H}-CDR3 of the V_{H}-CDR1, V_{H}-CDR2, V_{H}-CDR3 within SEQ ID NO: 448 and the V_{L}-CDR1, V_{L}-CDR2, V_{L}-CDR3 of the V_{L}-CDR1, V_{L}-CDR2, V_{L}-CDR3 within SEQ ID NO: 462;

26) the V_{H}-CDR1, V_{H}-CDR2, V_{H}-CDR3 of the V_{H}-CDR1, V_{H}-CDR2, V_{H}-CDR3 within SEQ ID NO: 449 and the V_{L}-CDR1, V_{L}-CDR2, V_{L}-CDR3 of the V_{L}-CDR1, V_{L}-CDR2, V_{L}-CDR3 within SEQ ID NO: 463;

27) the V_{H}-CDR1, V_{H}-CDR2, V_{H}-CDR3 of the V_{H}-CDR1, V_{H}-CDR2, V_{H}-CDR3 within SEQ ID NO: 450 and the V_{L}-CDR1, V_{L}-CDR2, V_{L}-CDR3 of the V_{L}-CDR1, V_{L}-CDR2, V_{L}-CDR3 within SEQ ID NO: 464;

28) the V_{H}-CDR1, V_{H}-CDR2, V_{H}-CDR3 of the V_{H}-CDR1, V_{H}-CDR2, V_{H}-CDR3 within SEQ ID NO: 756 and the V_{L}-CDR1, V_{L}-CDR2, V_{L}-CDR3 of the V_{L}-CDR1, V_{L}-CDR2, V_{L}-CDR3 within SEQ ID NO: 784;

29) the V_{H}-CDR1, V_{H}-CDR2, V_{H}-CDR3 of the V_{H}-CDR1, V_{H}-CDR2, V_{H}-CDR3 within SEQ ID NO: 757 and the V_{L}-CDR1, V_{L}-CDR2, V_{L}-CDR3 of the V_{L}-CDR1, V_{L}-CDR2, V_{L}-CDR3 within SEQ ID NO: 785;

30) the V_{H}-CDR1, V_{H}-CDR2, V_{H}-CDR3 of the V_{H}-CDR1, V_{H}-CDR2, V_{H}-CDR3 within SEQ ID NO: 758 and the V_{L}-CDR1, V_{L}-CDR2, V_{L}-CDR3 of the V_{L}-CDR1, V_{L}-CDR2, V_{L}-CDR3 within SEQ ID NO: 786;

31) the V_{H}-CDR1, V_{H}-CDR2, V_{H}-CDR3 of the V_{H}-CDR1, V_{H}-CDR2, V_{H}-CDR3 within SEQ ID NO: 759 and the V_{L}-CDR1, V_{L}-CDR2, V_{L}-CDR3 of the V_{L}-CDR1, V_{L}-CDR2, V_{L}-CDR3 within SEQ ID NO: 787;

32) the V_{H}-CDR1, V_{H}-CDR2, V_{H}-CDR3 of the V_{H}-CDR1, V_{H}-CDR2, V_{H}-CDR3 within SEQ ID NO: 760 and the V_{L}-CDR1, V_{L}-CDR2, V_{L}-CDR3 of the V_{L}-CDR1, V_{L}-CDR2, V_{L}-CDR3 within SEQ ID NO: 788;

33) the V_{H}-CDR1, V_{H}-CDR2, V_{H}-CDR3 of the V_{H}-CDR1, V_{H}-CDR2, V_{H}-CDR3 within SEQ ID NO: 761 and the V_{L}-CDR1, V_{L}-CDR2, V_{L}-CDR3 of the V_{L}-CDR1, V_{L}-CDR2, V_{L}-CDR3 within SEQ ID NO: 789;

34) the V_{H}-CDR1, V_{H}-CDR2, V_{H}-CDR3 of the V_{H}-CDR1, V_{H}-CDR2, V_{H}-CDR3 within SEQ ID NO: 762 and the V_{L}-CDR1, V_{L}-CDR2, V_{L}-CDR3 of the V_{L}-CDR1, V_{L}-CDR2, V_{L}-CDR3 within SEQ ID NO: 790;

35) the V_{H}-CDR1, V_{H}-CDR2, V_{H}-CDR3 of the V_{H}-CDR1, V_{H}-CDR2, V_{H}-CDR3 within SEQ ID NO: 763 and the V_{L}-CDR1, V_{L}-CDR2, V_{L}-CDR3 of the V_{L}-CDR1, V_{L}-CDR2, V_{L}-CDR3 within SEQ ID NO: 791;

36) the V_{H}-CDR1, V_{H}-CDR2, V_{H}-CDR3 of the V_{H}-CDR1, V_{H}-CDR2, V_{H}-CDR3 within SEQ ID NO: 764 and the V_{L}-CDR1, V_{L}-CDR2, V_{L}-CDR3 of the V_{L}-CDR1, V_{L}-CDR2, V_{L}-CDR3 within SEQ ID NO: 792;

37) the V_{H}-CDR1, V_{H}-CDR2, V_{H}-CDR3 of the V_{H}-CDR1, V_{H}-CDR2, V_{H}-CDR3 within SEQ ID NO: 765 and the V_{L}-CDR1, V_{L}-CDR2, V_{L}-CDR3 of the V_{L}-CDR1, V_{L}-CDR2, V_{L}-CDR3 within SEQ ID NO: 793;

38) the V_{H}-CDR1, V_{H}-CDR2, V_{H}-CDR3 of the V_{H}-CDR1, V_{H}-CDR2, V_{H}-CDR3 within SEQ ID NO: 766 and the V_{L}-CDR1, V_{L}-CDR2, V_{L}-CDR3 of the V_{L}-CDR1, V_{L}-CDR2, V_{L}-CDR3 within SEQ ID NO: 794;

39) the V_{H}-CDR1, V_{H}-CDR2, V_{H}-CDR3 of the V_{H}-CDR1, V_{H}-CDR2, V_{H}-CDR3 within SEQ ID NO: 767 and the V_{L}-CDR1, V_{L}-CDR2, V_{L}-CDR3 of the V_{L}-CDR1, V_{L}-CDR2, V_{L}-CDR3 within SEQ ID NO: 795;

40) the V_{H}-CDR1, V_{H}-CDR2, V_{H}-CDR3 of the V_{H}-CDR1, V_{H}-CDR2, V_{H}-CDR3 within SEQ ID NO: 768 and the V_{L}-CDR1, V_{L}-CDR2, V_{L}-CDR3 of the V_{L}-CDR1, V_{L}-CDR2, V_{L}-CDR3 within SEQ ID NO: 796;

41) the V_{H}-CDR1, V_{H}-CDR2, V_{H}-CDR3 of the V_{H}-CDR1, V_{H}-CDR2, V_{H}-CDR3 within SEQ ID NO: 769 and the V_{L}-CDR1, V_{L}-CDR2, V_{L}-CDR3 of the V_{L}-CDR1, V_{L}-CDR2, V_{L}-CDR3 within SEQ ID NO: 797;

42) the V_{H}-CDR1, V_{H}-CDR2, V_{H}-CDR3 of the V_{H}-CDR1, V_{H}-CDR2, V_{H}-CDR3 within SEQ ID NO: 770 and the V_{L}-CDR1, V_{L}-CDR2, V_{L}-CDR3 of the V_{L}-CDR1, V_{L}-CDR2, V_{L}-CDR3 within SEQ ID NO: 798;

43) the V_{H}-CDR1, V_{H}-CDR2, V_{H}-CDR3 of the V_{H}-CDR1, V_{H}-CDR2, V_{H}-CDR3 within SEQ ID NO: 771 and the V_{L}-CDR1, V_{L}-CDR2, V_{L}-CDR3 of the V_{L}-CDR1, V_{L}-CDR2, V_{L}-CDR3 within SEQ ID NO: 799;

44) the V_{H}-CDR1, V_{H}-CDR2, V_{H}-CDR3 of the V_{H}-CDR1, V_{H}-CDR2, V_{H}-CDR3 within SEQ ID NO: 772 and the V_{L}-CDR1, V_{L}-CDR2, V_{L}-CDR3 of the V_{L}-CDR1, V_{L}-CDR2, V_{L}-CDR3 within SEQ ID NO: 800;

45) the V_{H}-CDR1, V_{H}-CDR2, V_{H}-CDR3 of the V_{H}-CDR1, V_{H}-CDR2, V_{H}-CDR3 within SEQ ID NO: 773 and the V_{L}-CDR1, V_{L}-CDR2, V_{L}-CDR3 of the V_{L}-CDR1, V_{L}-CDR2, V_{L}-CDR3 within SEQ ID NO: 801;

46) the V_{H}-CDR1, V_{H}-CDR2, V_{H}-CDR3 of the V_{H}-CDR1, V_{H}-CDR2, V_{H}-CDR3 within SEQ ID NO: 774 and the V_{L}-CDR1, V_{L}-CDR2, V_{L}-CDR3 of the V_{L}-CDR1, V_{L}-CDR2, V_{L}-CDR3 within SEQ ID NO: 802;

47) the V_{H}-CDR1, V_{H}-CDR2, V_{H}-CDR3 of the V_{H}-CDR1, V_{H}-CDR2, V_{H}-CDR3 within SEQ ID NO: 775 and the V_{L}-CDR1, V_{L}-CDR2, V_{L}-CDR3 of the V_{L}-CDR1, V_{L}-CDR2, V_{L}-CDR3 within SEQ ID NO: 803;

48) the V_{H}-CDR1, V_{H}-CDR2, V_{H}-CDR3 of the V_{H}-CDR1, V_{H}-CDR2, V_{H}-CDR3 within SEQ ID NO: 776 and the V_{L}-CDR1, V_{L}-CDR2, V_{L}-CDR3 of the V_{L}-CDR1, V_{L}-CDR2, V_{L}-CDR3 within SEQ ID NO: 804;

49) the V_{H}-CDR1, V_{H}-CDR2, V_{H}-CDR3 of the V_{H}-CDR1, V_{H}-CDR2, V_{H}-CDR3 within SEQ ID NO: 777 and the V_{L}-CDR1, V_{L}-CDR2, V_{L}-CDR3 of the V_{L}-CDR1, V_{L}-CDR2, V_{L}-CDR3 within SEQ ID NO: 805;

50) the V_{H}-CDR1, V_{H}-CDR2, V_{H}-CDR3 of the V_{H}-CDR1, V_{H}-CDR2, V_{H}-CDR3 within SEQ ID NO: 778 and the V_{L}-CDR1, V_{L}-CDR2, V_{L}-CDR3 of the V_{L}-CDR1, V_{L}-CDR2, V_{L}-CDR3 within SEQ ID NO: 806;

51) the V_{H}-CDR1, V_{H}-CDR2, V_{H}-CDR3 of the V_{H}-CDR1, V_{H}-CDR2, V_{H}-CDR3 within SEQ ID NO: 779 and the V_{L}-CDR1, V_{L}-CDR2, V_{L}-CDR3 of the V_{L}-CDR1, V_{L}-CDR2, V_{L}-CDR3 within SEQ ID NO: 807;

52) the V_{H}-CDR1, V_{H}-CDR2, V_{H}-CDR3 of the V_{H}-CDR1, V_{H}-CDR2, V_{H}-CDR3 within SEQ ID NO: 780 and the V_{L}-CDR1, V_{L}-CDR2, V_{L}-CDR3 of the V_{L}-CDR1, V_{L}-CDR2, V_{L}-CDR3 within SEQ ID NO: 808;

53) the V_{H}-CDR1, V_{H}-CDR2, V_{H}-CDR3 of the V_{H}-CDR1, V_{H}-CDR2, V_{H}-CDR3 within SEQ ID NO: 781 and the V_{L}-CDR1, V_{L}-CDR2, V_{L}-CDR3 of the V_{L}-CDR1, V_{L}-CDR2, V_{L}-CDR3 within SEQ ID NO: 809;

54) the V_{H}-CDR1, V_{H}-CDR2, V_{H}-CDR3 of the V_{H}-CDR1, V_{H}-CDR2, V_{H}-CDR3 within SEQ ID NO: 782 and the V_{L}-CDR1, V_{L}-CDR2, V_{L}-CDR3 of the V_{L}-CDR1, V_{L}-CDR2, V_{L}-CDR3 within SEQ ID NO: 810; or

55) the V_{H}-CDR1, V_{H}-CDR2, V_{H}-CDR3 of the V_{H}-CDR1, V_{H}-CDR2, V_{H}-CDR3 within SEQ ID NO: 783 and the V_{L}-CDR1, V_{L}-CDR2, V_{L}-CDR3 of the V_{L}-CDR1, V_{L}-CDR2, V_{L}-CDR3 within SEQ ID NO: 811.

265. The use of any one of arrangements 252-264, wherein the antibody or binding fragment comprises:

1) the heavy chain variable region of SEQ ID NO: 136 and the light chain variable region of SEQ ID NO: 161;

2) the heavy chain variable region of SEQ ID NO: 137 and the light chain variable region of SEQ ID NO: 162;

3) the heavy chain variable region of SEQ ID NO: 138 and the light chain variable region of SEQ ID NO: 163;

4) the heavy chain variable region of SEQ ID NO: 139 and the light chain variable region of SEQ ID NO: 164;

5) the heavy chain variable region of SEQ ID NO: 139 and the light chain variable region of SEQ ID NO: 171;

6) the heavy chain variable region of SEQ ID NO: 140 and the light chain variable region of SEQ ID NO: 165;

7) the heavy chain variable region of SEQ ID NO: 141 and the light chain variable region of SEQ ID NO: 166;

8) the heavy chain variable region of SEQ ID NO: 142 and the light chain variable region of SEQ ID NO: 167;

9) the heavy chain variable region of SEQ ID NO: 143 and the light chain variable region of SEQ ID NO: 168;

10) the heavy chain variable region of SEQ ID NO: 144 and the light chain variable region of SEQ ID NO: 169;

11) the heavy chain variable region of SEQ ID NO: 145 and the light chain variable region of SEQ ID NO: 170;

12) the heavy chain variable region of SEQ ID NO: 146 and the light chain variable region of SEQ ID NO: 172;

13) the heavy chain variable region of SEQ ID NO: 148 and the light chain variable region of SEQ ID NO: 174;

14) the heavy chain variable region of SEQ ID NO: 436 and the light chain variable region of SEQ ID NO: 451;

15) the heavy chain variable region of SEQ ID NO: 438 and the light chain variable region of SEQ ID NO: 453;

16) the heavy chain variable region of SEQ ID NO: 439 and the light chain variable region of SEQ ID NO: 162;

17) the heavy chain variable region of SEQ ID NO: 440 and the light chain variable region of SEQ ID NO: 454;

18) the heavy chain variable region of SEQ ID NO: 441 and the light chain variable region of SEQ ID NO: 455;

19) the heavy chain variable region of SEQ ID NO: 442 and the light chain variable region of SEQ ID NO: 456;

20) the heavy chain variable region of SEQ ID NO: 443 and the light chain variable region of SEQ ID NO: 457;

21) the heavy chain variable region of SEQ ID NO: 444 and the light chain variable region of SEQ ID NO: 458;

22) the heavy chain variable region of SEQ ID NO: 445 and the light chain variable region of SEQ ID NO: 459;

23) the heavy chain variable region of SEQ ID NO: 446 and the light chain variable region of SEQ ID NO: 460;

24) the heavy chain variable region of SEQ ID NO: 447 and the light chain variable region of SEQ ID NO: 461;

25) the heavy chain variable region of SEQ ID NO: 448 and the light chain variable region of SEQ ID NO: 462;

26) the heavy chain variable region of SEQ ID NO: 449 and the light chain variable region of SEQ ID NO: 463;

27) the heavy chain variable region of SEQ ID NO: 450 and the light chain variable region of SEQ ID NO: 464;

28) the heavy chain variable region of SEQ ID NO: 756 and the light chain variable region of SEQ ID NO: 784;

29) the heavy chain variable region of SEQ ID NO: 757 and the light chain variable region of SEQ ID NO: 785;

30) the heavy chain variable region of SEQ ID NO: 758 and the light chain variable region of SEQ ID NO: 786;

31) the heavy chain variable region of SEQ ID NO: 759 and the light chain variable region of SEQ ID NO: 787;

32) the heavy chain variable region of SEQ ID NO: 760 and the light chain variable region of SEQ ID NO: 788;

33) the heavy chain variable region of SEQ ID NO: 761 and the light chain variable region of SEQ ID NO: 789;

34) the heavy chain variable region of SEQ ID NO: 762 and the light chain variable region of SEQ ID NO: 790;

35) the heavy chain variable region of SEQ ID NO: 763 and the light chain variable region of SEQ ID NO: 791;

36) the heavy chain variable region of SEQ ID NO: 764 and the light chain variable region of SEQ ID NO: 792;

37) the heavy chain variable region of SEQ ID NO: 765 and the light chain variable region of SEQ ID NO: 793;

38) the heavy chain variable region of SEQ ID NO: 766 and the light chain variable region of SEQ ID NO: 794;

39) the heavy chain variable region of SEQ ID NO: 767 and the light chain variable region of SEQ ID NO: 795;

40) the heavy chain variable region of SEQ ID NO: 768 and the light chain variable region of SEQ ID NO: 796;

41) the heavy chain variable region of SEQ ID NO: 769 and the light chain variable region of SEQ ID NO: 797;

42) the heavy chain variable region of SEQ ID NO: 770 and the light chain variable region of SEQ ID NO: 798;

43) the heavy chain variable region of SEQ ID NO: 771 and the light chain variable region of SEQ ID NO: 799;

44) the heavy chain variable region of SEQ ID NO: 772 and the light chain variable region of SEQ ID NO: 800;

45) the heavy chain variable region of SEQ ID NO: 773 and the light chain variable region of SEQ ID NO: 801;

46) the heavy chain variable region of SEQ ID NO: 774 and the light chain variable region of SEQ ID NO: 802;

47) the heavy chain variable region of SEQ ID NO: 775 and the light chain variable region of SEQ ID NO: 803;

48) the heavy chain variable region of SEQ ID NO: 776 and the light chain variable region of SEQ ID NO: 804;

49) the heavy chain variable region of SEQ ID NO: 777 and the light chain variable region of SEQ ID NO: 805;

50) the heavy chain variable region of SEQ ID NO: 778 and the light chain variable region of SEQ ID NO: 806;

51) the heavy chain variable region of SEQ ID NO: 779 and the light chain variable region of SEQ ID NO: 807;

52) the heavy chain variable region of SEQ ID NO: 780 and the light chain variable region of SEQ ID NO: 808;

53) the heavy chain variable region of SEQ ID NO: 781 and the light chain variable region of SEQ ID NO: 809;

54) the heavy chain variable region of SEQ ID NO: 782 and the light chain variable region of SEQ ID NO: 810; or

55) the heavy chain variable region of SEQ ID NO: 783 and the light chain variable region of SEQ ID NO: 811.

266. The use of any one of arrangements 252-265, wherein the anti-Gal3 antibody or binding fragment thereof is selected from the group consisting of TB001, TB006, 12G5.D7, 13A12.2E5, 14H10.2C9, 15F10.2D6, 19B5.2E6, 20D11.2C6, 20H5.A3, 23H9.2E4, 2D10.2B2, 3B11.2G2, 7D8.2D8, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 847.14H4, 846T.1H2, mIMT001, 4A11.2B5, 4A11.H1L1, 4A11.H4L2, 4G2.2G6, 6B3.2D3, 6H6.2D6, 9H2.2H10, 13G4.2F8, 13H12.2F8, 15G7.2A7, 19D9.2E5, 23B10.2B12, 24D12.2H9, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, or a binding fragment thereof.

267. The use of any one of arrangements 252-266, wherein the anti-Gal3 antibody or binding fragment is selected from the group consisting of: 6H6.2D6, 20H5.A3, 20D11.2C6, 4G2.2G6, 13H12.2F8, 19B5.2E6, 15G7.2A7, 23H9.2E4, 19D9.2E5, 2D10.2B2, 4A11.2B5, 14H10.2C9, 3B11.2G2, 13A12.2E5, 7D8.2D8, 15F10.2D6, 23B10.2B12, 12G5.D7, 24D12.2H9, 6B3.2D3, 13G4.2F8, 9H2.2H10, 846.1F5, 846.2H3, 846T.1H2, IMT-001, 4A11.H3L1, 4A11.H1L1 and 4A11.H4L2, or a binding fragment thereof.

268. The use of any one of arrangements 252-267, wherein the anti-Gal3 antibody or binding fragment is 2D10.2B2 or 6H6.2D6, or a binding fragment thereof.

269. The use of any one of arrangements 252-268, wherein the anti-Gal3 antibody or binding fragment is selected from the group consisting of 2D10.2B2 and 6H6.2D6, or a binding fragment thereof.

270. The use of any one of arrangements 252-269, wherein the anti-Gal3 antibody or binding fragment is used as a supplement to a standard of care treatment.

271. The use of arrangement 270, wherein the standard of care treatment comprises surgery, radiation, chemotherapy, targeted therapy, immunotherapy, a PD1/PDL1 blockade therapy, a CTLA4 blockade therapy, temozolomide, or any combination thereof.

272. An antibody or binding fragment thereof that binds to an N-terminal domain and/or the TRD of Gal3.

273. An antibody or binding fragment thereof that binds to an epitope present within a region of Gal3, wherein the epitope comprises:

| | (a) Peptide 1 |
|---|---|
| SEQ ID NO: 3 | |
| | (ADNFSLHDALSGSGNPNPQG;); |
| | (b) Peptide 6 |
| SEQ ID NO: 8 | |
| | (GAYPGQAPPGAYPGAPGAYP;); |
| | or |
| | (c) Peptide 7 |
| SEQ ID NO: 9 | |
| | (AYPGAPGAYPGAPAPGVYPG;); |

or any combination thereof.

274. The antibody or binding fragment thereof of arrangement 272 or 273, wherein the antibody or binding fragment thereof is 2D10.2B2, 6H6.2D6, or a binding fragment thereof.

275. The antibody or binding fragment thereof of arrangement 272 or 273, wherein the antibody or binding fragment thereof is selected from the group consisting of 2D10.2B2 and 6H6.2D6, or a binding fragment thereof.

276. The anti-Gal3 antibody or binding fragment thereof of any of the preceding arrangements for use in assisting a payload to cross a blood brain barrier of a subject.

277. The use of arrangement 276, wherein the subject has a neurological disorder.

278. A protein comprising one or more peptide sequences having at least 80%, 85%, 90%, 95%, 99%, or 100% homology to one or more of the peptide sequences of FIG. 18-27.

279. The protein of arrangement 278, wherein the protein is an antibody or binding fragment thereof.

280. The protein of arrangement 278 or 279, comprising:

a) a V_{H}-CDR1 peptide sequence having at least 80%, 85%, 90%, 95%, 99%, or 100% homology to one or more of the peptide sequences of FIG. 18;

b) a V_{H}-CDR2 peptide sequence having at least 80%, 85%, 90%, 95%, 99%, or 100% homology to one or more of the peptide sequences of FIG. 19;

c) a V_{H}-CDR3 peptide sequence having at least 80%, 85%, 90%, 95%, 99%, or 100% homology to one or more of the peptide sequences of FIG. 20;

d) a V_{L}-CDR1 peptide sequence having at least 80%, 85%, 90%, 95%, 99%, or 100% homology to one or more of the peptide sequences of FIG. 21;

e) a V_{L}-CDR2 peptide sequence having at least 80%, 85%, 90%, 95%, 99%, or 100% homology to one or more of the peptide sequences of FIG. 22;

f) a V_{L}-CDR3 peptide sequence having at least 80%, 85%, 90%, 95%, 99%, or 100% homology to one or more of the peptide sequences of FIG. 23;

g) a heavy chain variable region peptide sequence having at least 80%, 85%, 90%, 95%, 99%, or 100% homology to one or more of the peptide sequences of FIG. 24;

h) a light chain variable region peptide sequence having at least 80%, 85%, 90%, 95%, 99%, or 100% homology to one or more of the peptide sequences of FIG. 25;

i) a heavy chain peptide sequence having at least 80%, 85%, 90%, 95%, 99%, or 100% homology to one or more of the peptide sequences of FIG. 26;

j) a light chain peptide sequence having at least 80%, 85%, 90%, 95%, 99%, or 100% homology to one or more of the peptide sequences of FIG. 27;

or any combination thereof.

281. The protein of any one of arrangements 278-280, comprising a peptide sequence having at least 80%, 85%, 90%, 95%, 99%, or 100% homology to a peptide sequence encoded by any one or more of the nucleic acid sequences of FIG. 37-40.

282. The protein of any one of arrangements 278-281, wherein the protein is an antibody or binding fragment thereof that binds to Gal3.

In some embodiments, the anti-Gal3 antibodies or binding fragments thereof described herein are able to efficiently traverse the blood-brain barrier of an animal such as a human. These anti-Gal3 antibodies are conjugated to one or more payloads, such as cytotoxic payloads, microtubule disrupting agents, DNA modifying agents, Akt inhibitors, polymerase inhibitors, detectable moieties, immunomodulatory agents, immune modulators, immunotoxins, nucleic acid polymers, aptamers, peptides, or any combination thereof. These payloads may be those that do not efficiently cross the blood-brain barrier or have a low permeability to the blood-brain barrier, where conjugating the payloads to the anti-Gal3 antibodies or binding fragments thereof increases the permeability of the payload across the blood-brain barrier.

In some embodiments, patients present with or are suspected of having a neurological disease, such as inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, or brain cancer (primary or secondary brain tumors), or any combination thereof.

In some embodiments, antibody conjugates comprising any one of the anti-Gal3 antibodies or binding fragments thereof and one or more payloads disclosed herein can be administered as doses at an amount of 1 ng (or in the alternative: 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans). The doses are administered every 1 day (or in the alternative: every 2, 3, 4, 5, 6, 7, 8, 9, 10, 11, 12, 24, 36, or 48 days or weeks or any time within a range defined by any two of the aforementioned times).

In some embodiments, in the case of payloads intended to have a physiological effect on the patients, an improvement of the neurological disease or symptoms associated with the neurological disease is observed in the patients following administration of the administration of the antibody conjugate.

In some embodiments, in the case of payloads intended to be used as a diagnostic or detection moiety, the payload experiences increased permeation to nervous system tissue by virtue of being conjugated to the anti-Gal3 antibody or binding fragment thereof, which aids in the detection, identification, and/or quantification of the target of the diagnostic or detectable payload. In some embodiments, the target may be an abnormal growth or lesion of brain tissue, such as for brain cancer or neurodegenerative conditions.

In some embodiments, any of the Gal-3 antibodies provided herein can cross the blood brain barrier of a subject who has one or more of the disorders provided herein (e.g., Alzheimer's disease, etc.). In some embodiments, the blood-brain barrier of such subjects can be compromised compared to a healthy subject, and the one or more of the present antibodies can cross such a compromised barrier.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1. GAL3 Specifically Binds to APP695

To evaluate the possibility that human galectin-3 (GAL3) could physically interact with human amyloid precursor protein (APP), ELISA assessments with purified GAL3 and APP isoform APP695 were conducted. Human GAL3 protein (mammalian, R&D Systems, 8259-GA; E. coli derived, untagged, TrueBinding, QCB200349; or His-tagged, TrueBinding, QCB200352) was diluted in PBS (Corning, 21-030-CM) to a concentration of 4, 2, or 1 μg/mL and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. APP695 (R&D Systems, 9937-AP) was biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 4, 2, or 1 μg/mL of biotinylated recombinant APP695 protein in 2% BSA in PBST was added to the wells. The plate was incubated for an hour at room temperature with gentle rocking. Thereafter, the plate was washed three times with PBST. Avidin HRP (Biolegend, 405103; 1:2000 dilution) was diluted in 2% BSA in PBST and then added to the wells. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

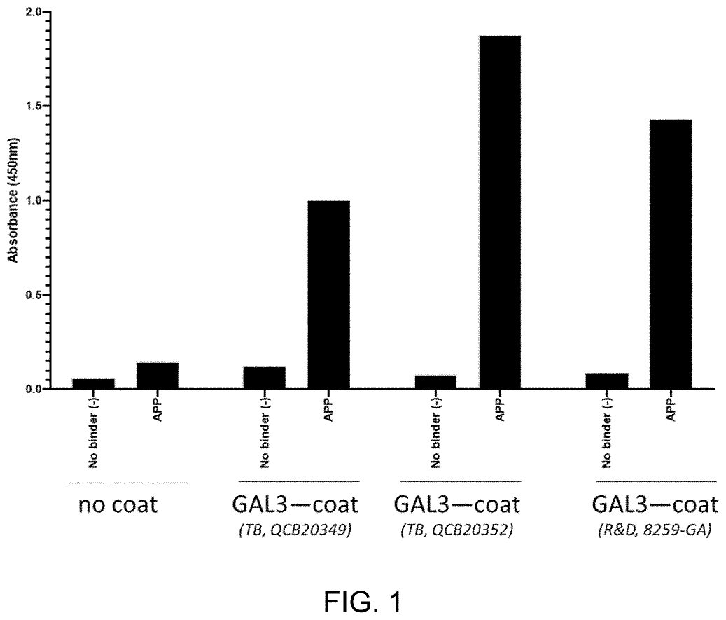

As depicted in FIG. 1, APP695 protein strongly bound GAL3-coated wells. APP695 protein did not significantly bind to uncoated ELISA wells. Likewise, no significant binding signal was observed in wells with only GAL3 coating.

### Example 2. Identification of GAL3-Binding Antibodies with APP695-Gal3 Blocking Activity

To identify Gal3-targeted antibodies with the ability to block the interaction of Gal3 and APP695, purified Gal3 and APP695 proteins were incubated in the presence (or absence) of various Gal3-targeted or control antibodies, or without antibody, and protein interaction was evaluated by ELISA.

Human GAL3 (mammalian, R&D Systems, 8259-GA; E. coli derived, untagged, TrueBinding, QCB200349; or His-tagged, TrueBinding, QCB200352) was diluted in PBS (Corning, 21-030-CM) to a concentration of 4 μg/mL and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Human APP695 (R&D Systems, 9937-AP) was biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 30 μL of IgG4 isotype control or anti-GAL3 antibodies (FIG. 32) at 20, 6.6, or 2.2 μg/ml were added to each well, followed by the addition of 30 μL of 2, 3, or 4 μg/ml biotinylated recombinant APP695 protein in 2% BSA in PBST. The plate was incubated for an hour at room temperature with gentle rocking. Thereafter, the plate was washed three times with PBST. Avidin HRP (Biolegend, 405103) was then added to the wells at 1:2000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm. Percent blockade of Gal3-APP695 interaction was calculated as the fraction of signal obtained in the absence of antibody with the background signal subtracted.

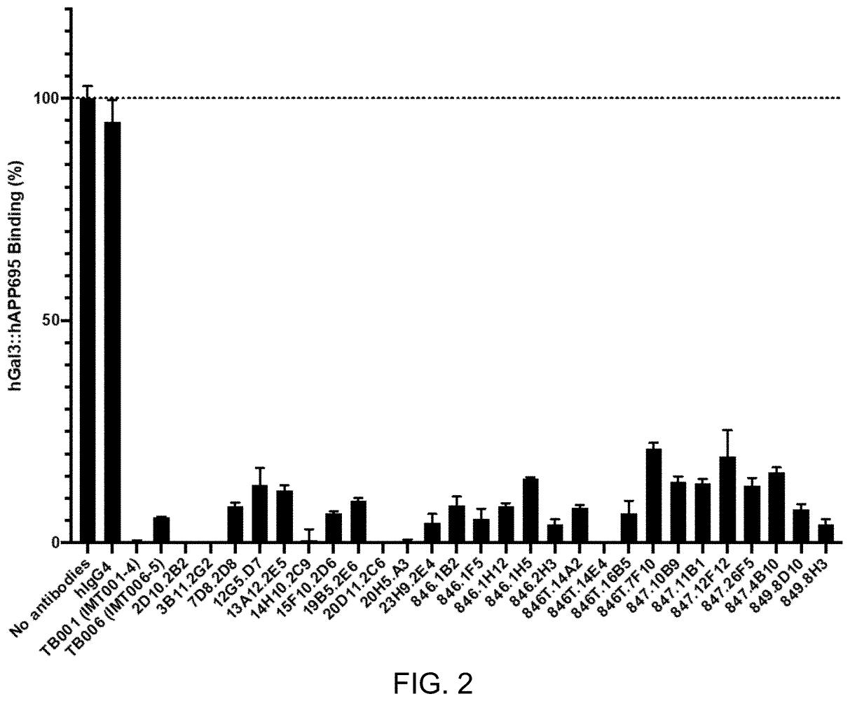

Twenty-nine anti-Gal3 antibodies were assayed. As shown in FIG. 2, anti-Gal3 antibodies exhibited differential ability to block the interaction of Gal3 and APP695. Twenty antibodies (19B5.2E6, 7D8.2D8, F846C.1B2, F846C.1H12, F846TC.14A2, F849C.8D10, F849C.8H3, 4A11.H3L1 [IMT006-5 (TB006)], 15F10.2D6, F846TC.16B5, 23H9.2E4, F846C.1F5, IMT001-4 [TB001], F846C.2H3, 14H10.2C9, 15FG7.2A7, 20H5.A3, F846TC.14E4, 3B11.2G2, 20D11.2C6, 2D10.2B2) disrupted Gal3-APP695 binding, resulting a reduction in Gal3-APP695 binding to >90% of unblocked control (no antibody), respectively. Antibodies 13G4.2F8, F846TC.7F10, F847C.12F12, and F847C.4B10 moderately disrupted the Gal3-APP695 binding, reducing the interaction to 45-85% of unblocked controls, respectively. Finally, six antibodies (6B3.2D3, F849C.1D2, F849C.3H2, F849C.5H1, F849C.8D12, and 24D12.2H9) did not impact Gal3-APP695 binding (not shown). Anti-Gal3 antibodies display differential ability to block hGAL3 interaction with APP695, therefore, Gal3 binding alone is not sufficient to disrupt the interaction of Gal3 and APP695, and specific properties were required for this disrupting activity.

### Example 3. APP695-GAL3 Antibodies with Blocking Activity Compete for Binding to GAL3

To determine whether GAL3 binding antibodies with APP695-GAL3 blocking activity bind to the same or overlapping regions of the GAL3 molecule, a large-scale antibody binning assay was performed to assess the ability of antibodies to simultaneously bind GAL3.

The epitope binning assay was done in a sandwich format on the high-throughput SPR-based Carterra LSA unit (CarterraBio, Salt Lake City, Utah). First, the purified antibodies were diluted to 10 μg/ml concentration in 10 mM NaOAc (pH 5.0) and then were covalently coupled via amine group to HC200M chip activated by EDC and S-NHS to immobilize antibodies to different positions of a 384-spot array. One hundred thirty-eight binning cycles were run on the array of immobilized antibodies. In each cycle, first, human Gal3 (AcroBio GA3-H5129) was injected over the entire array to bind to different antibodies (primary antibody), followed by one antibody (secondary antibody) selected among the panel of 150 anti-GAL3 antibodies tested. At the end of each cycle, the array was regenerated by 10 mM Glycine (pH 2.0) to remove bound antigen and secondary antibody from the array. The data analysis was done using the Epitope software by CarterraBio.

Binning results are shown in FIG. 33. In total, 49 distinct bins were identified for 120 anti-GAL3 antibodies that demonstrate binding to hGAL3 (30 antibodies out of 150 tested did not bind hGAL3 when immobilized on HC200M chip and were thus excluded from further analysis). Antibodies that strongly blocked the association of GAL3 and APP695 belonged to a number of distinct bins defined below. This highlights the utility of these bins as predictors of GAL3-APP695 blocking activity.

Antibody IMT001-4 [TB001] defined bin 1. Clones 4A11.H3L1 [IMT006-5 (TB006)], 19B5.2E6, 20H5.A3, 23H9.2E4, 2D10.2B2 exhibited mutual competitive binding for hGAL3, but did not prevent binding of the rest of the clones, thus defining bin 3. Clones 3B11.2G2, 13A12.2E5 exhibited mutual competitive binding for hGAL3, but did not prevent binding of the rest of the clones, thus defining bin 7. Clones 14H10.2C9, 15F10.2D6, 7D8.2D8, F846TC.14E4, F846TC.7F10, F849C.8D10 exhibited mutual competitive binding for hGAL3, but did not prevent binding of the rest of the clones, thus defining bin 8. Clones F846C.1B2, F846C.1F5, F846C.1H12, F846C.2H3, F846TC.16B5 exhibited mutual competitive binding for hGAL3, but did not prevent binding of the rest of the clones, thus defining bin 17. Clones F847C.10B9, F847C.12F12, and F847C.26F5 exhibited mutual competitive binding for hGAL3, but did not prevent binding of the rest of the clones, thus defining bin 49. In addition, a number of clones did not compete for binding to hGAL3 with other antibodies tested, therefore defining bin 5 (clone 20D11.2C6), bin 16 (clone 846.2B11), bin 10 (clone 12G5.D7), and bin 24 (clone 846.4D5), respectively.

### Example 4. GAL3 Antibodies with APP695-GAL3 Blocking Activity Bind to Distinct Epitopes of GAL3

To identify the epitopes to which Gal3 antibodies with and without Gal3-APP695 blocking activity bound, a library of 20 amino acid peptides representing portions of Gal3, summarized in FIG. 17 (SEQ ID NOs: 3-26), was produced and the ability to bind Gal3 antibodies was evaluated by ELISA.

Human Gal3 peptides (LifeTein, custom order) and human Gal3 proteins (R&D Systems, 8259-GA; TrueBinding, QCB200349) were diluted in PBS (Corning, 21-030-CM) to concentrations of at least 100 μg/mL (peptides) or 1 μg/mL (proteins), and added to the wells of several 96-well ELISA plates (Thermo Fisher, 44-2404-21). After incubating the plates at 4° C. overnight, the plates were washed three times with PBST (PBS with 0.05% Tween 20 [VWR, 0777]). The plates were then blocked for an hour with 2% BSA (EMD Millipore, 126609) in PBST at room temperature with gentle rocking. Thereafter, the 2% BSA in PBST was discarded and Gal3-binding antibodies (reformatted hIgG4 [S228P]) were diluted in 2% BSA in PBST to concentrations of 5 μg/mL and added to the wells. The plates were incubated for an hour at room temperature with gentle rocking and then washed three times with PBST. Afterwards, Peroxidase AffiniPure F(ab′)₂ Fragment Goat Anti-Human IgG (H+L) polyclonal antibody (Jackson ImmunoResearch, 109-036-003), was diluted in 2% BSA in PBST (1:4000) and added to the wells. The plates were incubated for 1 hour at room temperature with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

Peptide binding results are depicted in FIG. 33. Binding of Gal3-binding antibodies to the peptide array was observed at multiple locations, with the majority of binding observed in peptides 1-8 with some weak binding to peptide 17, summarized in FIG. 33. Significantly, all Gal3-binding antibodies with strong APP695-Gal3 blocking activity exhibited the ability to bind to peptides 1, 6, or 7, corresponding to peptide sequences in the N-terminal domain of Gal3. Specifically, 13 separate Gal3-binding antibodies with APP695-GAL3 blocking activity (19B5.2E6, 20D11.2C6, 20H5.A3, 23H9.2E4, 2D10.2B2, F846C.1H5, F846TC.14A2, F846TC.7F10, F847C.10B9, F847C.26F5, F847C.4B10, F847C.12F12, 15FG7.2A7) all bound peptide 1 of Gal3, corresponding to amino acids ADNFSLHDALSGSGNPNPQG (SEQ ID NO: 3) of Gal3. Similarly, 15 separate Gal3-binding antibodies with Gal3-APP695 blocking activity (4A11.H3L1 [TB006, IMT006-5], 13A12.2E5, 14H10.2C9, 20D11.2C6, 20H5.A3, 23H9.2E4, 2D10.2B2, 3B11.2G2, F846C.1B2, F846C.1F5, F846C.1H12, F846C.2H3, F846TC.16B5, 15FG7.2A7, and TB001 [IMT001-4]) bound peptide 6 of Gal3, corresponding to amino acids, GAYPGQAPPGAYPGAPGAYP (SEQ ID NO: 8) of Gal3. Further, 13 Gal3-binding antibodies with Gal3-APP695 blocking activity (14H10.2C9, 19B5.2E6, 20D11.2C6, 20H5.A3, 23H9.2E4, 2D10.2B2, 3B11.2G2, F846C.1B2, F846TC.14A2, F847C.10B9, F847C.26F5, F847C.12F12, 15FG7.2A7) all bound peptide 7 of Gal3, corresponding to amino acids AYPGAPGAYPGAPAPGVYPG (SEQ ID NO: 9) of Gal3.

### Example 5. GAL3 Antibodies with APP695-GAL3 Blocking Activity Improve Cognitive Function in an Alzheimer's Disease Transgenic Mouse Model (APPSwe)

To address whether GAL3 antibodies with APP695-GAL3 blocking activity have therapeutic potential in APPSwe animals harboring an Aβ plaque, APPSwe mice were treated with anti-GAL3 antibodies or isotype control (twice a week for two weeks, at 10 mg/kg concentration). The antibodies were injected intraperitoneally (IP). After 4 doses, animals underwent hippocampal dependent spatial behavior tests (Morris Water Maze). Wild type un-injected C57BL/6 mice were used as a control cohort.

The APPSwe mice (APPSwe, Model:1349) were purchased from Taconic Biosciences and control wild type (WT) animals (C57BL/6J, stock number: 000664) were from Jackson Laboratory (Bar Harbor, Me., USA). Animals were housed and maintained on a 12/12 h light/dark cycle (light on at 8:00 A.M.) with food and water continuously available. Experimental procedures followed the guidelines of Animal Use and Care of the National Institute of Health (NIH) and were approved by the Animal Committee of TrueBinding, Inc.

To investigate cognitive function, hippocampal dependent Morris Water Maze was employed before and after the treatment. The apparatus used for all water maze tasks was a circular aluminum tank (1.5 m diameter) painted white and filled with water maintained at 26° C.-29° C. The maze was located in a room containing simple visual, extra-maze cues. To reduce stress, mice were placed on the platform in both the hidden and cued versions of the task for 15 sec. prior to the first training trial. Mice were trained to swim to a circular clear Plexiglas platform (14 cm diameter) submerged 1.5 cm beneath the surface of the water and invisible to the mice while swimming. The platform location was selected randomly at before and after treatment and was kept constant for each individual mouse throughout training. On each trial, the mouse was placed into the tank at one of four designated start points in a pseudorandom order. Mice were allowed 60 seconds to find the submerged platform. If a mouse failed to find the platform within 60 seconds, it was manually guided to the platform and allowed to remain there for 15 seconds. After this, each mouse was placed into a holding cage under a warming lamp for 30 seconds before initiation of the next trial. To ensure that memory differences were not due to lack of task learning, mice were given four trials a day for as many days as were required to train the APPSwe mice and control WT (C57BL/6J) mice to reach the criterion (<20 seconds). The animals were trained for 5-6 days. Retention of the spatial training was assessed 24 hours after the last training trial. Probe trial consisted of a 60 seconds free swim in the pool without the platform. Mice were monitored by a camera mounted in the ceiling directly above the pool to record the 24-hours test. The parameters measured during the probe trial included initial latency to cross the platform location, number of platform location crosses.

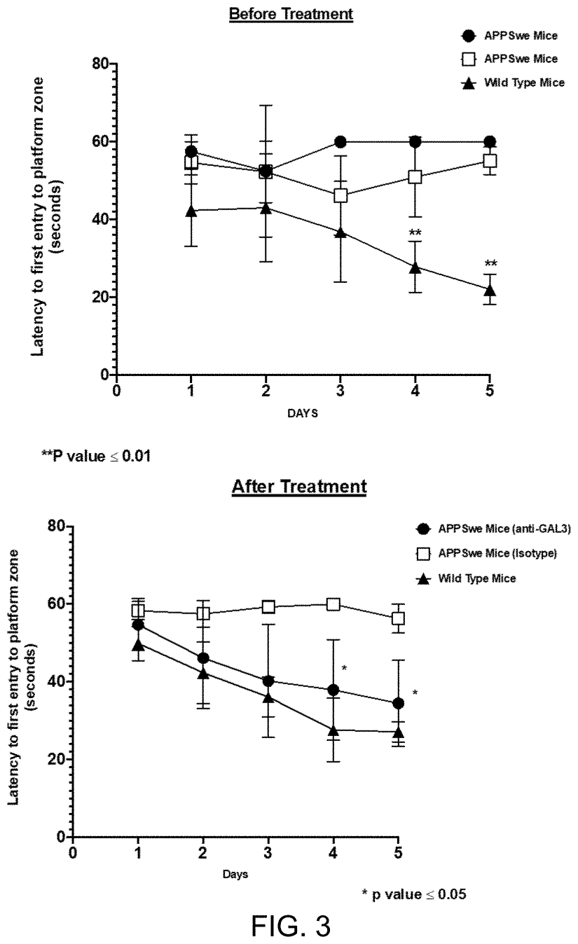

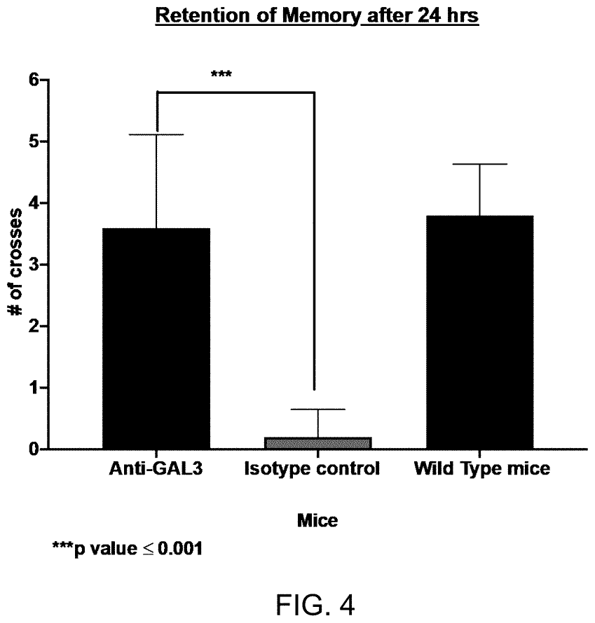

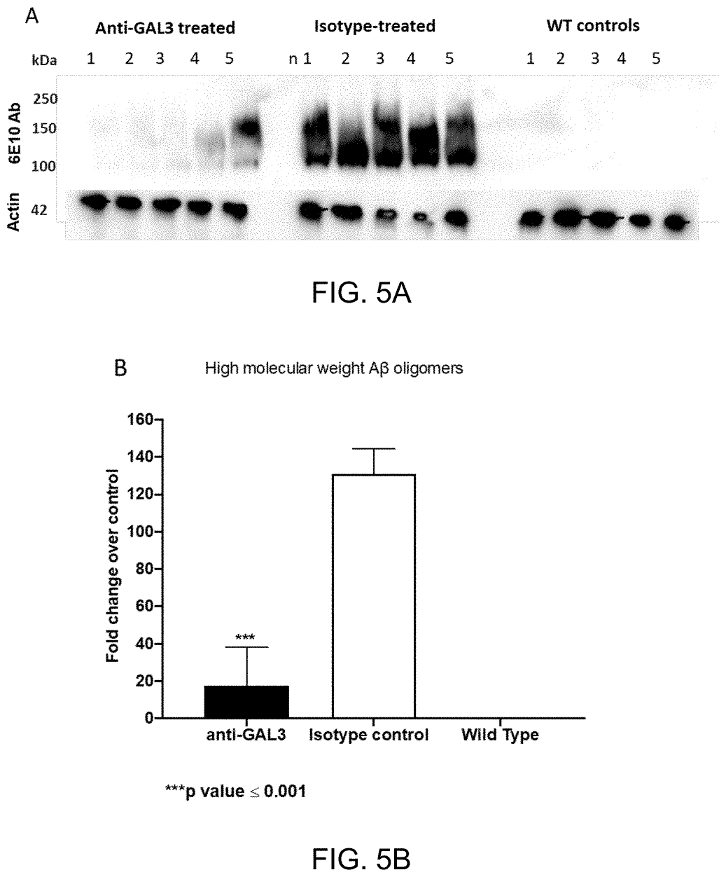

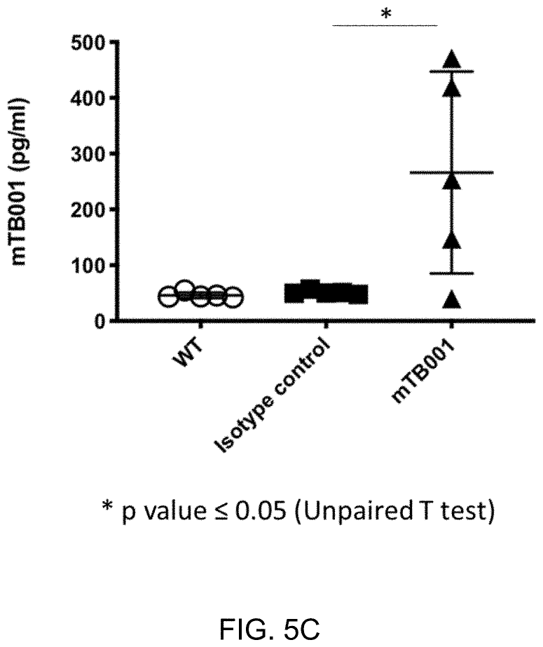

All the APPSwe transgenic mice (n=10) demonstrated a significant deficit (**p<0.01) in the latency of the Morris Water Maze test as compared to wild type C57BL/6J mice (n=5) prior to initiation of treatment as shown in FIG. 3. Based on behavioral performance, mice were randomized and divided in two groups. Ten-month-old APPSwe mice were dosed with anti-GAL3 antibodies (TB001) and isotype control (MOPC21) antibody. At the end of the treatment period, the behavioral phenotype of the APPSwe mice was evaluated using the Morris Water Maze as described above. APPswe mice dosed with anti-GAL3 (TB001) antibody showed significant improvement in latency to cross the platform as compared to isotype control dosed APPSwe mice as shown in FIG. 3. At 24 h probe trials, mice treated with TB001 showed significant improvement (***p<0.001) in retention memory as evident by their improved latency to cross the platform location (not shown) and the number of platform crosses during the 24-hour tests as compared with isotype control dosed APPSwe mice as shown in FIG. 4. The behavioral data were analyzed using Graph Pad prism. All values are reported as mean±SEM and significance set at p<0.05. The additional marker of AD progression in APPSwe animals is extracellular Aβ deposits that are apparent by 4-6 months of age in the frontal cortex and become more extensive by twelve months. Western blot analysis using Aβ specific monoclonal antibody 6E10 (Biolegend) was used to detect structural aggregates of Aβ was performed in the brain of APPSwe and wild type control mice. Brain was homogenized briefly using a hand homogenizer in RIPA buffer containing protease inhibitor cocktail. Samples were centrifuged at 14,000 rpm for 1 hour at 4° C. and supernatants were collected. Protein concentrations of the brain lysates were measured with the Pierce 660-nm protein assay reagent (Thermo Scientific, Rockford, Ill.). The samples were boiled in sodium dodecyl sulfate-polyacrylamide gel electrophoresis (SDS-PAGE) sample buffer. Equal amounts of protein from each fraction (20 μg) were resolved by SDS-PAGE and transferred onto polyvinylidene difluoride membrane. To detect the total Aβ levels, Aβ sequence specific antibody 6E10 was used and the immunoreactive bands were visualized with an enhanced chemiluminescence reagent. β-Actin was used as a loading control and images were quantified using image ISO lite software. The total Aβ levels were significantly higher in isotype control (MOPC21) dosed APPSwe mice. Treatment with TB001 shows significant reduction in higher molecular weight Aβ oligomers ranging from 100-150 kDa molecular weight detected by 6E10 antibody when compared to isotype-treated transgenic mice (***p<0.001), quantified by Image J software (FIG. 5). As expected, no Aβ was detected in wild type mice. Therefore, anti-GAL3 treatment not only improves the cognitive function in APPSwe mice, but also attenuates the accumulation of toxic conformational species of AD.

To determine the levels of anti-GAL3 antibodies in the brain tissue of antibody-treated APPswe mice, a soluble fraction of brain lysates was prepared. Briefly, frozen hemibrains collected from the treated animals were homogenized using a hand homogenizer in RIPA buffer containing protease inhibitor cocktail. Samples were centrifuged at 14,000 rpm for 1 hour at 4° C. After centrifugation, soluble supernatant was collected and pellet was frozen down (insoluble fraction). Protein concentrations of the brain lysates were measured with the Pierce 660-nm protein assay reagent (Thermo Scientific, Rockford, Ill.). The levels of anti-GAL3 antibodies (mTB001) samples were determined by ELISA-based assay. The plates were coated overnight at 4° C. with 1 μg/ml of hGAL3 (in-house reagent, QC200348). Twenty-four hours after incubation, the plates were blocked with PBST containing 2% BSA for 2 hr at room temperature. Each brain tissue lysate was diluted three-fold. One hundred (100) μl of each sample was tested per well. Each sample was analyzed in triplicates. Following two-hour incubation at room temperature, the plates were washed in PBS, and HRP-conjugated goat anti-mouse IgG was added. Following additional incubation for one-hour, the plates were developed with TMB. The reaction was stopped by addition of 1M HCL. Known concentrations of mTB001 were added to brain tissue lysates collected from naïve mice and served as internal standard curve. mTB001 antibody was detected ten days after the last treatment in the brain samples of mTB001-treated mice (FIG. 5C).

### Example 6. GAL3 Antibodies with APP695-GAL3 Blocking Activity Improve Cognitive Function in Aβ-Induced Neurodegeneration in C57BL/6 Mice

To address whether GAL3 antibodies with APP695-GAL3 blocking activity have therapeutic potential in Aβ42-injected mice, Aβ42 fibril-injected animals were treated with anti-GAL3 antibodies or isotype control (twice a week for two weeks, at 10 mg/kg concentration). The antibodies were injected (IP). After 4 doses, animals underwent hippocampal dependent spatial behavior test (Morris Water Maze). Wild type un-injected C57BL/6 mice were used as a control cohort.

To generate Aβ42-injected animals, Aβ42 fibrils were prepared as follows. One (1) mg of the lyophilized Aβ42 peptide was resuspended in 90 μL of 100 mM NaOH and incubated for 10 minutes. This solution was then diluted to a final concentration of 0.5 mg/mL by adding 10 mM sodium phosphate buffer (pH 7.4). The solution was continuously stirred during the aggregation time course using a stir bar. The solution was stirred for 7 days. 2 μL of sample was pipetted onto a Whatman nitrocellulose membrane at the appropriate time points (between 0 and 7 day-time points) for dot blot, and 20 μL of each appropriate time point was frozen down for sodium dodecyl sulphate-polyacrylamide gel electrophoresis (SDS-PAGE) to confirm formation of the higher molecular weight Aβ42 fibrils. The formed Aβ42 fibrils (0.2 μg) were injected into the brains of C57BL/6J mice (n=15) by stereotaxic injection using 26 G stainless steel needles and Hamilton syringe. The injection was incorporated with the following co-ordinates: −1.0±0.06 mm posterior to bregma, 1.8±0.1 mm lateral to the sagittal suture, and 2.4 mm in depth. The mice were postoperatively monitored to check for any signs of infection or illness. After three weeks, hippocampal dependent cognitive function test was employed.

Cognitive function test (Morris Water Maze) was employed before and after the treatment. The apparatus used for all water maze tasks was a circular aluminum tank (1.5 m diameter) painted white and filled with water maintained at 26° C.-29° C. The maze was located in a room containing simple visual, extra-maze cues. To reduce stress, mice were placed on the platform in both the hidden and cued versions of the task for 15 sec. prior to the first training trial. Mice were trained to swim to a circular clear Plexiglas platform (14 cm diameter) submerged 1.5 cm beneath the surface of the water and invisible to the mice while swimming. The platform location was selected randomly before and after treatment and was kept constant for each individual mouse throughout training. On each trial, the mouse was placed into the tank at one of four designated start points in a pseudorandom order. Mice were allowed 60 seconds to find the submerged platform. If a mouse failed to find the platform within 60 seconds, it was manually guided to the platform and allowed to remain there for 15 seconds. After this, each mouse was placed into a holding cage under a warming lamp for 30 seconds before initiation of the next trial. To ensure that memory differences were not due to lack of task learning, mice were given four trials a day for as many days as were required to train the C57BL/6J mice to reach the criterion (<20 seconds). The animals were trained for 5-6 days. Retention of the spatial training was assessed 24 hours after the last training trial. Probe trial consisted of a 60 seconds free swim in the pool without the platform. Mice were monitored by a camera mounted in the ceiling directly above the pool to record the 24-hours test. The parameters measured during the probe trial included initial latency to cross the platform location, number of platform location crosses.

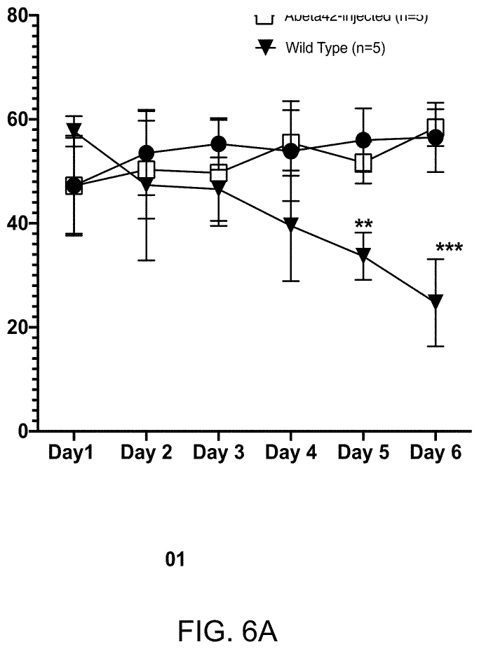

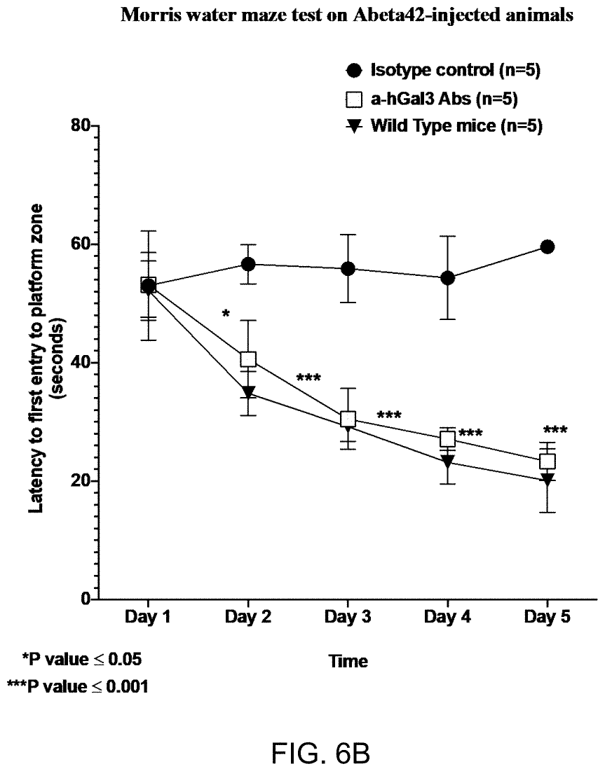

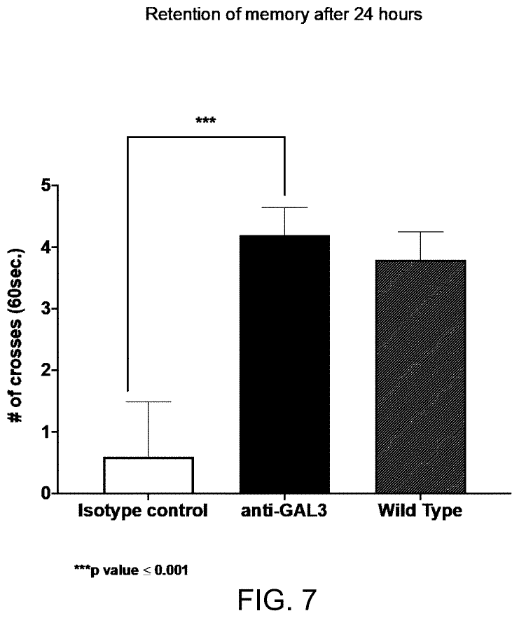

C57BL/6J mice injected with the fibrillar form of Aβ42 peptide demonstrated significant deficits in cognitive function of hippocampal dependent spatial memory (e.g. learning to find the platform in the Morris Water Maze and remembering the location of the platform) as compared to age matched un-injected C57BL/6 mice (***p≤0.001). Based on behavioral performance before the treatment, mice were randomized and divided in groups as shown in FIG. 6A. The animals were treated with anti-GAL3 antibodies and isotype control via IP administration (twice a week for two weeks, at 10 mg/kg concentration). At the end of the treatment period, behavioral phenotype of mice was evaluated using the Morris Water Maze test, and anti-GAL3-antibody-treated mice showed significant improvement in latency to cross the platform as compared to isotype control (MOPC21) treated groups as shown in FIG. 6B. At 24 h probe trials, mice treated with four doses of mTB001 showed significant improvement (***p≤0.001) in retention memory as evident by their improved latency to cross the platform location and the number of platform crosses during the 24 h tests as shown in FIG. 7. The behavioral data were analyzed using Graph pad prism-8. All values are reported as mean±SEM and significance set at p<0.05.

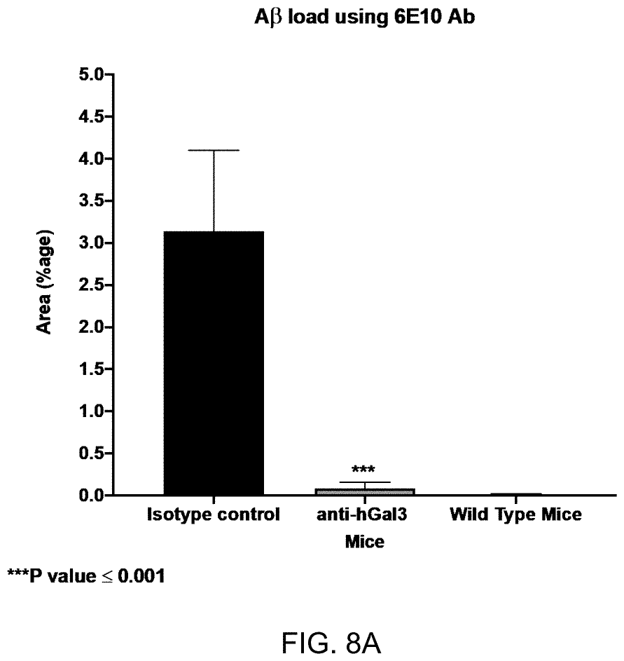

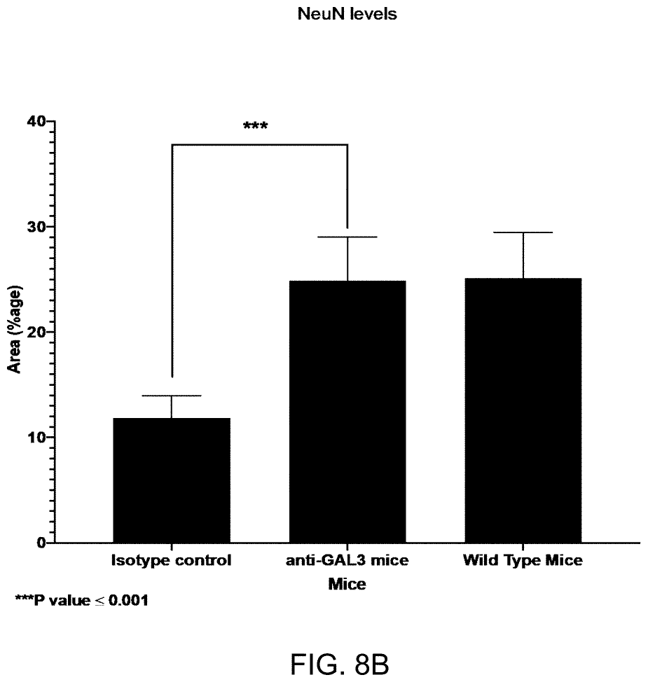

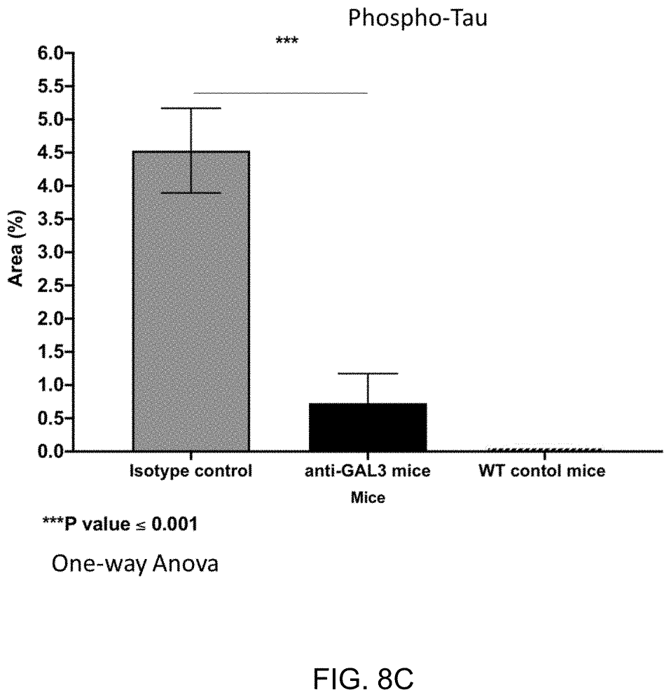

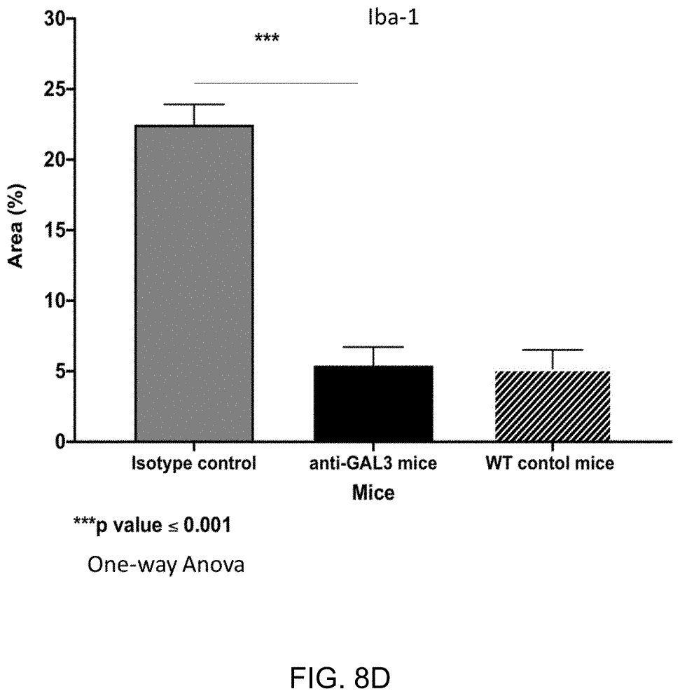

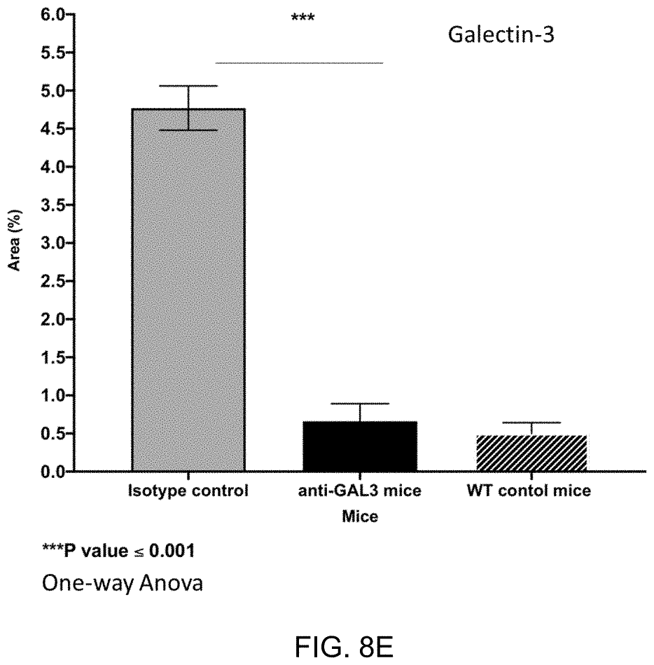

To further evaluate the therapeutic effect of anti-GAL3 antibodies in Aβ42-injected models, tissue samples were collected from the animals and analyzed by immunohistochemistry. Mice were anesthetized with isoflurane, blood was collected by cardiac puncture, and mice were perfused transcardially with cold phosphate-buffered saline (PBS). For the comparison, mice subjected to TB001 and control isotype treatment along with wild type mice were sacrificed. Brain tissues were fixed overnight with 4% paraformaldehyde in PBS (pH 7.4) at 4° C. and stored in PBS/0.02% sodium azide (NaN₃) at 4° C. Fixed brain tissues were sectioned (40 pm) with a vibratome. Coronal sections were collected in PBS (containing 0.02% sodium azide) and stored at 4° C. prior to staining. To stain for Aβ plaques, sections were immersed in 70% formic acid for 5 minutes (min). Endogenous peroxidase in tissue was blocked by treating with 3% H₂O₂ in PBS for 10 min at 25° C. Nonspecific background staining was blocked by 1 hour incubation in 2% bovine serum albumin, 0.3% Triton X-100 (TX) at 25° C. Tissues were incubated with primary Aβ sequence specific antibody (6E10, Biolegend) overnight at 4° C., rinsed three times with PBS, 0.1% TX, followed by biotinylated secondary antibody (anti-mouse) detection with an ABC peroxidase kit, and visualization with a 3,3′-diaminobenzidine (DAB) substrate kit (Vector, Burlingame, Calif., USA). After DAB staining, brain tissues were mounted on Superfrost plus microscopic slides (Thermofisher) and dehydrated using different percentages of alcohols and xylene. Slides were cover-slipped using DPX (Sigma) mounting media. Brain tissues were scanned using Aperio VERSA Brightfield, Fluorescence & FISH Digital Pathology Scanner (Leica Biosystems). In addition to improving cognitive function, the anti-GAL3 treatment improved plaque burden in mice. Total amyloid deposition was assessed by quantifying the amount of anti-AD (6E10) immunoreactive material. Image analysis of sections from multiple animals demonstrated that Aβ deposits in anti-GAL3 (TB001)-treated mice were significantly reduced (***p<0.001) as compared with isotype-dosed mice as shown in FIG. 8A. NIH Image J software was used to analyze the immunohistochemistry and quantification was done using Graph Pad Prism 8.

To determine the outcome of anti-Gal3 antibody treatment on neuronal regeneration, the brain samples from mice were analyzed for the levels of NeuN, a specific marker of neurons. The samples were processed as described above using the anti-NeuN antibody EPR12763 (ab177487, Abcam). As shown in FIG. 8B, anti-Gal3 antibody treatment significantly increased the number of neurons in brain tissue of Aβ42-injected mice determined by NeuN levels.

To determine the outcome of anti-Gal3 antibody treatment on the levels of Tau phosphorylation, the brain samples from mice were analyzed using phospho-Tau specific antibody (Ser202, Thr205, AT8, ThermoFisher #MN1020). The phosphor-Tau levels were reduced by anti-Gal3 antibody treatment (FIG. 8C).

Furthermore, the levels of activated microglia (detected by anti-Iba-1 antibody [WAKO 013-27691]) as well as Galectin-3 levels (detected by anti-GAL3 antibody [Cedarlane, CL8942AP]) were diminished in the brain samples collected from anti-GAL3 treated animals (FIGS. 8D and 8E, respectively).

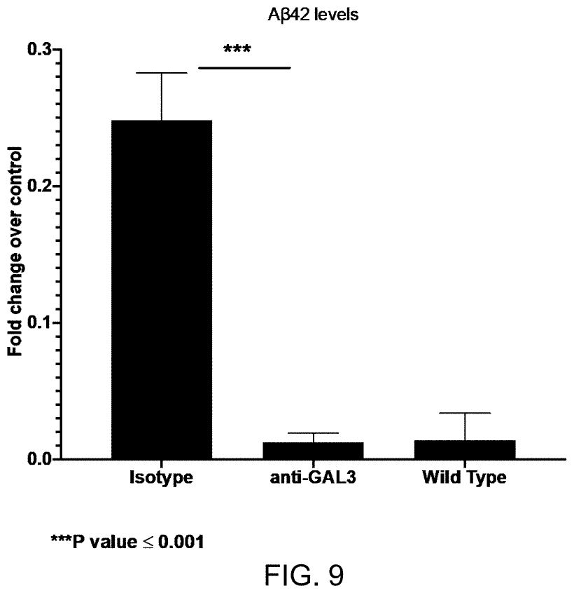

Extracellular Aβ deposits are an additional marker of AD progression in Aβ42-injected models. Western blot analysis using Aβ specific monoclonal antibody 6E10 (Biolegend) to detect structural aggregates of Aβ was performed in the brain of Aβ42-injected model and wild type control mice. Brain tissue samples were collected from animals following behaviour test. The samples were homogenized briefly using a hand homogenizer in RIPA buffer containing protease inhibitor cocktail. Samples were further centrifuged at 14,000 rpm for one hour at 4° C. and supernatants were collected. Protein concentrations of the brain lysates were measured with the Pierce 660-nm protein assay reagent (Thermo Scientific, Rockford, Ill.). The samples were boiled in sodium dodecyl sulfate-polyacrylamide gel electrophoresis (SDS-PAGE) sample buffer. Equal amounts of protein from each fraction (20 μg) were resolved by SDS-PAGE and transferred onto polyvinylidene difluoride membrane. To detect the total Aβ levels, sequence specific antibody 6E10 (Biolegend) was used, the immunoreactive bands were visualized with an enhanced chemiluminescence reagent. β-Actin was used as a loading control and images were quantified using image ISO lite software. Treatment with anti-GAL3 antibodies (TB001) resulted in significant reduction in higher molecular weight Aβ oligomers ranging from 100-150 kDa molecular weight detected by 6E10 antibody when compared to isotype-treated transgenic mice (***p<0.001, by one-way ANOVA), quantified by Image J software (FIG. 9). As expected, no Aβ was detected in wild type mice. Therefore, anti-GAL3 treatment not only improve the cognitive function in the Aβ42-injected model, but also attenuates the accumulation of toxic conformational species of AD.

### Example 7. GAL3 Antibodies with APP695-GAL3 Blocking Activity Promote Phagocytic Function of Microglia

To demonstrate the activity of anti-GAL3 antibodies to promote the clearance of Aβ plaques by microglia, the activity of in-house antibodies is validated in a microglia phagocytosis assay. Briefly, immortalized murine BV-2 microglial cells are cultured in DMEM/F12 medium supplemented with 10% FBS and 1× Penicillin/Streptomycin. Aβ-42 fibrils and oligomers are labeled with Alexa Fluor 488 according to the manufacturer's instruction (Molecular Probes Invitrogen detection technologies, A30006). BV2 cells are seeded at 10,000/well in 96-well Flat Clear Bottom Black Polystyrene TC-treated Microplates (Sigma-Aldrich). Twenty-four hours later, the medium is changed to a serum-free medium (DMEM/F12), and the cells are incubated for three hours in a serum-free medium prior to the treatment with anti-GAL3 antibodies and isotype control (in a range of 0-30 μg/mL). Following one-hour incubation, Alexa Fluor 488-labeled Aβ-42 fibrils and oligomers are added at 2.5 μM concentration. After 30 minutes incubation, the cells are washed and fixed with 4% paraformaldehyde at room temperature for 15 minutes. The cells' nuclei are stained by Hoechst 33342 (Invitrogen). The phagocytic efficiency is quantified by the AF488 fluorescence normalized to Hoechst fluorescence via Cytation (BioTek).

To further validate phagocytotic activity of microglial cells, the cells are pre-treated with anti-GAL3 antibodies and isotype control (in a range of 0-30 μg/mL) in the presence of unlabeled Aβ-42 fibrils and oligomers. Nile Red fluorescent microspheres (Life Technologies, F8819), as a marker of fluid phase phagocytosis, are added to the treated cells for 30 min. Cells are washed and fixed as described above. The phagocytosis efficiency is determined by the weighted average of ingested microspheres per cell.

Anti-GAL3 treatment promotes phagocytotic activity of microglial cells indicating the potential of anti-GAL3 antibodies with APP695-GAL3 blocking activity to control progression of Alzheimer's disease.

### Example 8. GAL3 Antibodies with APP695-GAL3 Blocking Activity Inhibit Aβ-42-Fibril-Mediated Activation of Microglia

To demonstrate the activity of anti-GAL3 antibodies to inhibit activation of microglia, immortalized murine microglial cells (BV2) are seeded at 25,000/well in 96-well flat clear bottom TC-treated microplates (Corning) for 24 hours. Next day, the cells are pre-treated with anti-GAL3 antibodies or relevant isotype control (at 0.3-30 μg/ml concentration) for one hour prior to activation by addition of Aβ-42 fibrils (0.1 μM to 10 μM concentration) prepared as described below. The conditioned media is collected twenty-four hours after the incubation with Aβ-42 fibrils and analyzed for cytokine secretion.

To generate Aβ-42 fibrils, one mg of the lyophilized Aβ42 peptide is resuspended in 90 μL of 100 mM NaOH and incubated for 10 minutes. This solution is then diluted to a final concentration of 0.5 mg/mL by adding 10 mM sodium phosphate buffer, pH 7.4. The solution is continuously stirred during the aggregation time course using a stir bar. To confirm the formation of Aβ42 fibrils, 2 μL of sample is pipetted onto a Whatman nitrocellulose membrane at the appropriate time points (0-7 days) for dot blot, and additional 20 μL of each sample is frozen down for sodium dodecyl sulphate-polyacrylamide gel electrophoresis (SDS-PAGE).

To assess activation of microglial cells twenty-four-hours following incubation with Aβ-42 fibrils, the TNF-α-levels in cell culture media are measured by ELISA-based assay (TNF-α detection kit [R&D], according to the manufacturer's protocol). Anti-GAL3 treatment significantly reduces AD-42-fibril-mediated activation of microglia as demonstrated by reduced production of TNF-α by BV-2 cells following the treatment.

### Example 9. GAL3 Antibodies with APP695-GAL3 Blocking Activity Inhibit the Formation of Aβ-42 Fibrils

To validate the efficacy of anti-GAL3 antibodies to interfere with AD-42-fibril formation, Aβ42 aggregation is performed according to the protocol of SensoLyte® Thioflavin T beta-amyloid (1-42) aggregation kit from AnaSpec (Catalog No. AS-72214, Fremont, Calif.). Ten microliters of 2 mM thioflavin-T working solution are mixed with 60 μL (15 μg) Aβ42 peptide solution (0.25 mg in 1 mL cold assay buffer) in each microplate well, then 1.25 μL (0.25 μg), 2.5 μL (0.5 μg), 5 μL (1 μg) and 10 μL (2 μg) of Gal-3 Ab are added to each well and to bring the total volume of all samples to 100 μL with assay buffer. One hundred microliters of the assay buffer are added as a blank. In brief, one set contains the thioflavin-T plus Aβ42 peptide solution and another set contains thioflavin-T plus Aβ42 peptide solution and increasing concentrations of Gal3 Ab. Fluorescence intensity is measured (Ex/Em=440/480 nm) immediately at 37° C. for 18 hrs. Anti-GAL3 antibodies significantly reduce Aβ-42-fibrils formation as apparent by reduced fluorescent emission in the antibody treated wells.

### Example 10. GAL3 Antibodies with APP695-GAL3 Blocking Activity Inhibit GAL3-Aβ-42 Peptide and Oligomer Interactions

To evaluate the possibility that human galectin-3 (GAL3) could physically interact with Amyloid beta (Abeta, AD), ELISA assessments with purified GAL3 and Abeta (1-42) peptide and oligomer were conducted. Human GAL3 protein (TrueBinding, QCB200349; TrueBinding, QCB200352) was diluted in PBS (Corning, 21-030-CM) to concentrations of 4, 2, and 1 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Human Abeta (1-42) was biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 4, 2, and 1 μg/ml of biotinylated Abeta (1-42) peptide (rPeptide, A-1117-1) or oligomer in 2% BSA in PBST was added to the wells. The plate was incubated for an hour at room temperature with gentle rocking. Thereafter, the plate was washed three times with PBST. Avidin HRP (Biolegend, 405103; 1:2000 dilution) was diluted in 2% BSA in PBST and then added to the wells. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

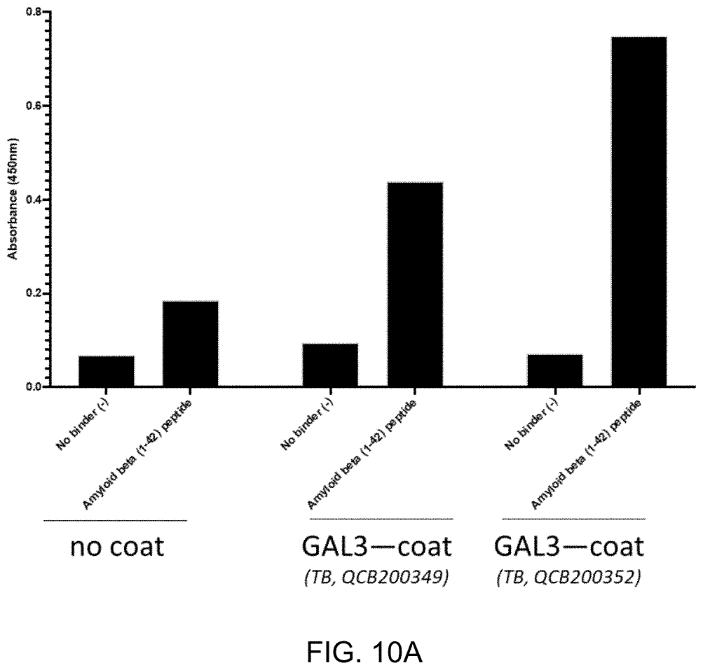

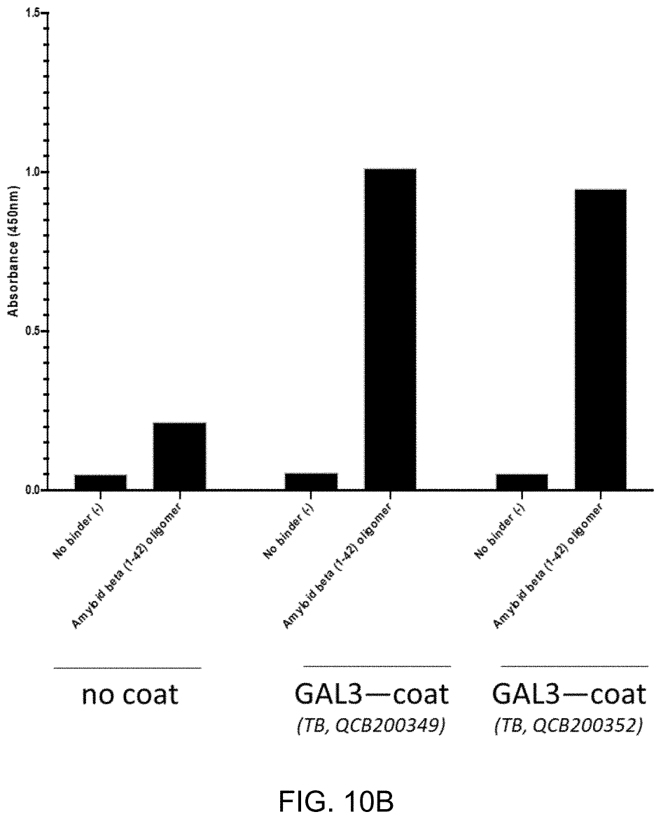

As depicted in FIG. 10A-B, Abeta (1-42) peptide (10A) and oligomer (10B) strongly bound GAL3-coated wells. Abeta peptide and oligomer did not significantly bind to uncoated ELISA wells. Likewise, no significant binding signal was observed in wells with only GAL3 coating.

To identify Gal3-targeted antibodies with the ability to block the interaction of Gal3 and Abeta, purified Gal3 and Abeta (1-42) peptide and oligomer were incubated in the presence (or absence) of various Gal3-targeted or control antibodies, or without antibody, and protein interaction was evaluated by ELISA.

Human GAL3 (TrueBinding, QCB200352) was diluted in PBS (Corning, 21-030-CM) to a concentration of 4 or 2 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Human Abeta (1-42) oligomer was biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 30 μl of control or anti-GAL3 antibodies at 20, 6.6, or 2.2 μg/ml was added to each well, followed by the addition of 30 μl of 8 μg/ml of biotinylated Abeta (1-42) peptide (rPeptide, A-1117-1) or 2 μg/ml of biotinylated Abeta (1-42) oligomer in 2% BSA in PBST. The plate was incubated for an hour at room temperature with gentle rocking. Thereafter, the plate was washed three times with PBST. Avidin HRP (Biolegend, 405103) was then added to the wells at 1:2000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm. Percent blockade of Gal3-Abeta (1-42) peptide or oligomer interaction was calculated as the fraction of signal obtained in the absence of antibody with the background signal subtracted.

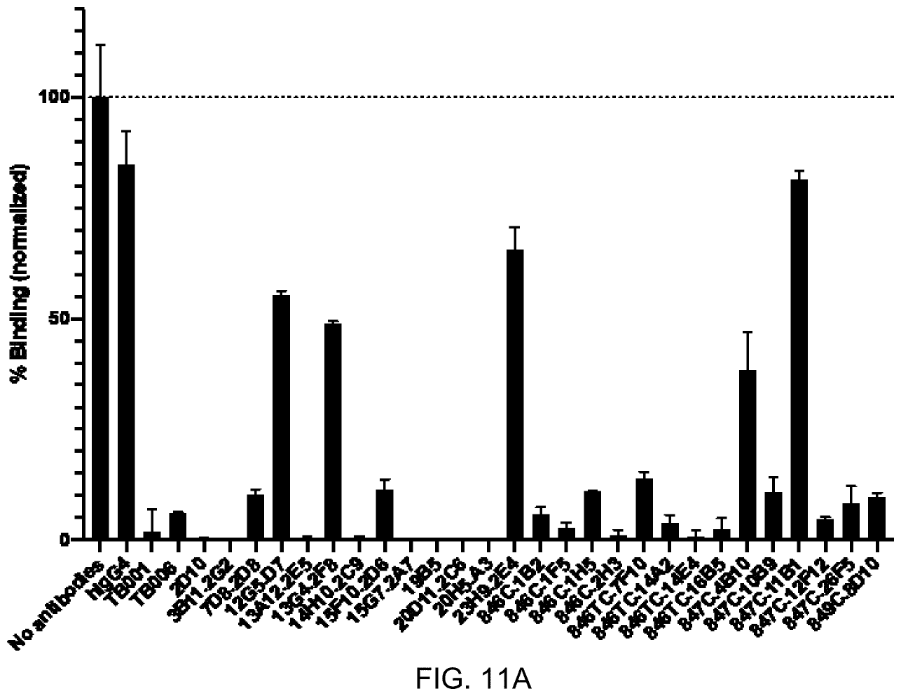

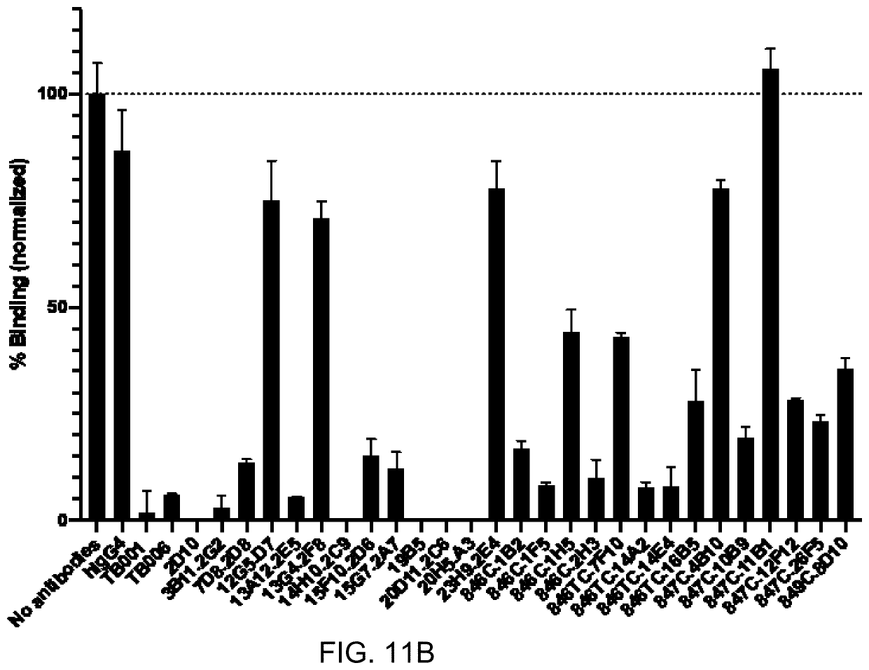

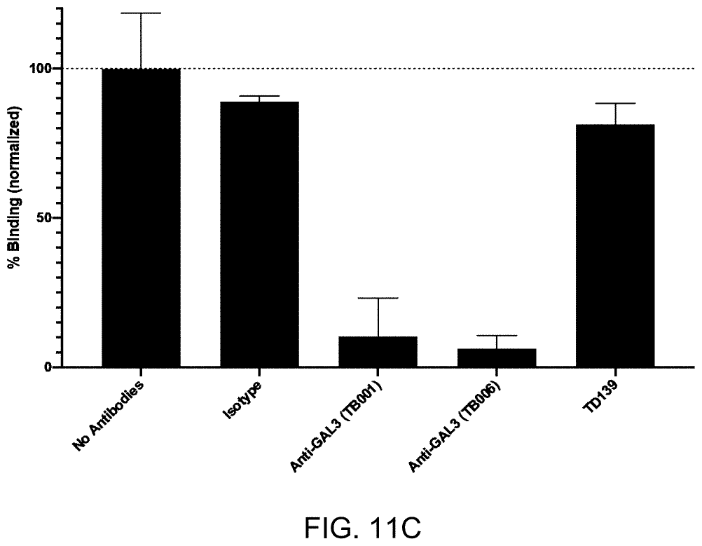

As shown in FIG. 11A-B, anti-Gal3 antibodies with APP695 blocking activities exhibited differential ability to block the interaction of Gal3 and Abeta (1-42) peptide (11A) and oligomer (11B). Of the nineteen antibodies that blocked Gal3-APP695 >90%, sixteen of them also blocked Gal3-Abeta peptide >90% (2D10.2B2 [2D10], 20D11.2C6, 3B11.2G2, 20H5.A3, 846TC.14E4, 15G7.2A7, 14H10.2C9, 846C.2H3, TB001 (IMT001-4), 846C.1F5, 846TC.16B5, TB006 (IMT006-5 [4A11.H3L1]), 846C.1B2, 846TC.14A2, 849C.8D10, 19B5) and eleven of them blocked Abeta oligomer >90% (2D10, 20D11.2C6, 3B11.2G2, 20H5.A3, 846TC.14E4, 14H10.2C9, TB001 (IMT001-4), 846C.1F5, TB006 (IMT006-5 [4A11.H3L1]), 846TC.14A2, 19B5). Interestingly, three antibodies (23H9.2E4, 12G5.D7, 847C.11B1) that displayed >86% blocking of Gal3-APP695 displayed <46% blocking of Gal3-Abeta peptide. Similarly, four antibodies (23H9.2E4, 12G5.D7, 847C.11B1, 847C.4B10) that displayed >83% blocking of Gal3-APP695 displayed <26% blocking of Gal3-Abeta oligomer. The six antibodies (849C.8D12, 849C.5H1, 6B3.2D3, 849C.3H2, 849C.1D2, 24D12.2H9) that displayed no blocking ability for Gal3-APP695 also displayed poor blocking ability for Abeta peptide and oligomer (data not shown). Anti-Gal3 antibodies display differential ability to block GAL3 interaction with Abeta peptide and oligomer, therefore, Gal3 binding alone is not sufficient to disrupt the interactions, and specific properties were required for this disrupting activity.

To compare the efficacy of anti-Gal3 antibodies with APP695 blocking activities and small molecule Gal3 inhibitor TD139 to block the interaction of Gal3 and Abeta (1-42) oligomer (Gal3-Abeta(1-42)Olig), Gal3-targeted antibodies, isotype control antibodies, and TD139 were evaluated in the Gal3-Abeta(1-42)Olig blocking assay by ELISA.

Briefly, human Gal3 (TrueBinding, QCB200352) was diluted in PBS (Corning, 21-030-CM) to a concentration of 2 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Human Abeta (1-42) oligomer was biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 30 μl of anti-Gal3 or isotype control antibodies (at 133.33, 44.44, or 14.81 nM), and Gal3 inhibitor TD139 (at 20,000, 6,666.67, or 2,222.22 nM) was added to each well, followed by the addition of 30 μl of 2 μg/ml of biotinylated Abeta (1-42) oligomer in 2% BSA in PBST. The plate was incubated for an hour at room temperature with gentle rocking. Thereafter, the plate was washed three times with PBST. Avidin HRP (Biolegend, 405103) was then added to the wells at 1:2000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm. Percent blockade of Gal3-Abeta (1-42) oligomer interaction was calculated as the fraction of signal obtained in the absence of antibody or inhibitor with the background signal subtracted.

As shown in FIG. 11C, in-house generated antibodies with APP695 blocking activities could block the interaction between hGal3 and Abeta oligomer more efficiently than Gal3 inhibitor TD139. At 66.67 nM, TB001 and TB006 could block Gal3-Abeta oligomer at 90 and 94%, respectively. This was much more efficient than TD139, which even at 10,000 nM, could only block Gal3-Abeta oligomer by at most 19%. The isotype control, at 66.67 nM, displayed limited blocking of Gal3-Abeta oligomer, as expected. The calculated IC50 values were 3.165 nM and 2.379 for TB001 and TB006, respectively, and not available to TD139.

### Example 11. GAL3 Antibodies with APP596-GAL3 Blocking Activity Inhibit GAL3-TLR4 Interaction

To evaluate the possibility that human galectin-3 (GAL3) could physically interact with human Toll-like Receptor 4 (TLR4), ELISA assessments with purified GAL3 and TLR4 were conducted. Human GAL3 protein (R&D Systems, 8259-GA; TrueBinding, untagged, E. coli-derived) was diluted in PBS (Corning, 21-030-CM) to concentrations of 4, 2, and 1 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Human TLR4 (PeproTech, 160-06) was biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 4, 2, and 1 μg/ml of biotinylated recombinant TLR4 protein in 2% BSA in PBST was added to the wells. The plate was incubated for an hour at room temperature with gentle rocking. Thereafter, the plate was washed three times with PBST. Avidin HRP (Biolegend, 405103; 1:2000 dilution) was diluted in 2% BSA in PBST and then added to the wells. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

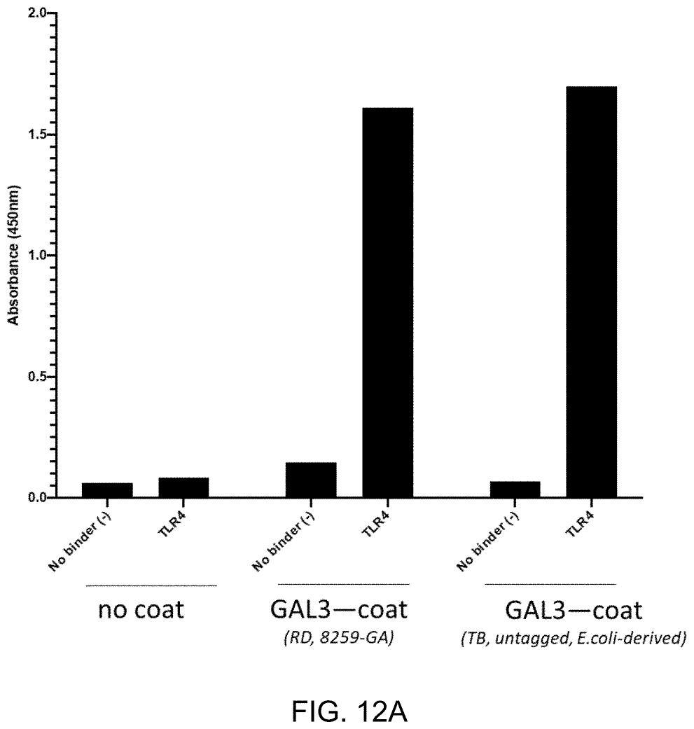

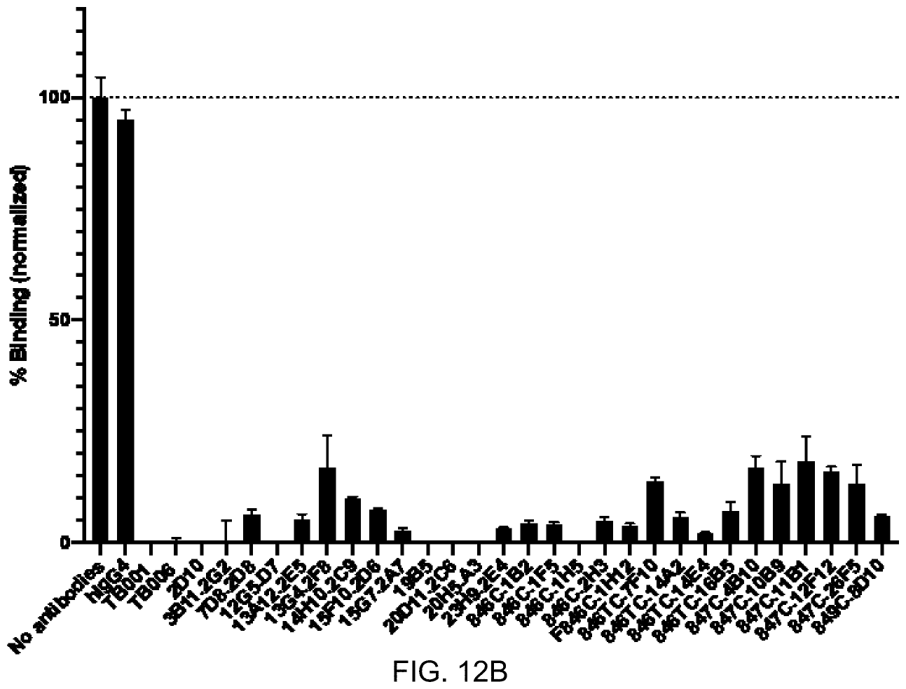

As depicted in FIG. 12A, TLR4 protein strongly bound GAL3-coated wells. TLR4 protein did not significantly bind to uncoated ELISA wells. Likewise, no significant binding signal was observed in wells with only GAL3 coating.

To identify Gal3-targeted antibodies with the ability to block the interaction of Gal3 and TLR4, purified Gal3 and TLR4 proteins were incubated in the presence (or absence) of various Gal3-targeted or control antibodies, or without antibody, and protein interaction was evaluated by ELISA.

Human GAL3 (TrueBinding, untagged, E. coli-derived or TrueBinding, QCB200349) was diluted in PBS (Corning, 21-030-CM) to a concentration of 4 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Human TLR4 (PeproTech, 160-06) was biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 30 μl of control or anti-GAL3 antibodies (FIG. 32) at 20, 6.6, or 2.2 μg/ml was added to each well, followed by the addition of 30 μl of 5, 6, or 8 μg/ml of biotinylated recombinant TLR4 protein in 2% BSA in PBST. The plate was incubated for an hour at room temperature with gentle rocking. Thereafter, the plate was washed three times with PBST. Avidin HRP (Biolegend, 405103) was then added to the wells at 1:2000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm. Percent blockade of Gal3-TLR4 interaction was calculated as the fraction of signal obtained in the absence of antibody with the background signal subtracted.

As shown in FIG. 12B, anti-Gal3 antibodies with APP695 blocking activities block the interaction of Gal3 and TLR4. Of the nineteen antibodies that blocked Gal3-APP695 >90%, eighteen of them also blocked Gal3-TLR4 >90% (2D10.2B2 [2D10], 20D11.2C6, 3B11.2G2, 20H5.A3, 846TC.14E4, 15G7.2A7, 846C.2H3, TB001 (IMT001-4), 23H9.2E4, 846C.1F5, 15F10.2D6, 846TC.16B5, TB006 (IMT006-5 [4A11.H3L1]), 7D8.2D8, 846C.1B2, 846TC.14A2, 849C.8D10, 19B5). Of the six antibodies (849C.8D12, 849C.5H1, 6B3.2D3, 849C.3H2, 849C.1D2, 24D12.2H9) that did not block Gal3-APP695, partial blocking between 33 and 63% was observed for Gal3-TLR4 (not shown). Finally, 846T.4C9 did not impact Gal3-TLR4 binding and was also a poor blocker of Gal3-APP695 (not shown). Anti-Gal3 antibodies display differential ability to block GAL3 interaction with TLR4, therefore, Gal3 binding alone is not sufficient to disrupt the interaction of Gal3 and TLR4, and specific properties are required for this disrupting activity. At lower concentrations, antibodies belonging to bin 17 and 49 are less efficient in blocking GAL3::TLR4 interaction than the rest in the list.

### Example 12. GAL3 Antibodies with APP596-GAL3 Blocking Activity Promote Inhibit GAL3-TREM2 Interaction

To evaluate the possibility that human galectin-3 (GAL3) could physically interact with human triggering receptor expressed on myeloid cells 2 (TREM2), ELISA assessments with purified GAL3 and TREM2 were conducted. Human GAL3 protein (R&D Systems, 8259-GA; TrueBinding, QCB200347; TrueBinding, untagged, E. coli-derived; TrueBinding, QCB200349) was diluted in PBS (Corning, 21-030-CM) to concentrations of 4, 2, and 1 μg/mL and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Thereafter, the 2% BSA in PBST was discarded and 4, 2, and 1 μg/mL of recombinant human TREM2 protein (Sino Biological, 11084-H08H) in 2% BSA in PBST was added to the wells. The plate was incubated for an hour at room temperature with gentle rocking. Thereafter, the plate was washed three times with PBST. Anti-6× His tag antibody-HRP (abcam, ab1269; 1:3000 dilution) was diluted in 2% BSA in PBST and then added to the wells. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

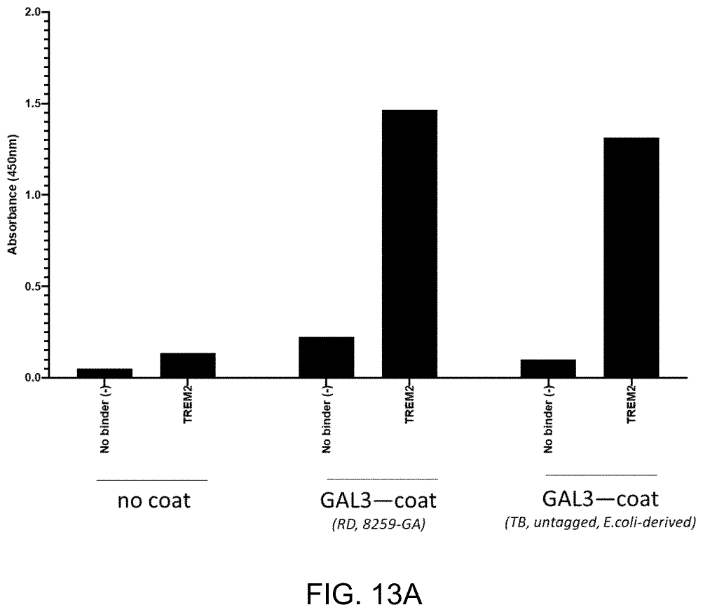

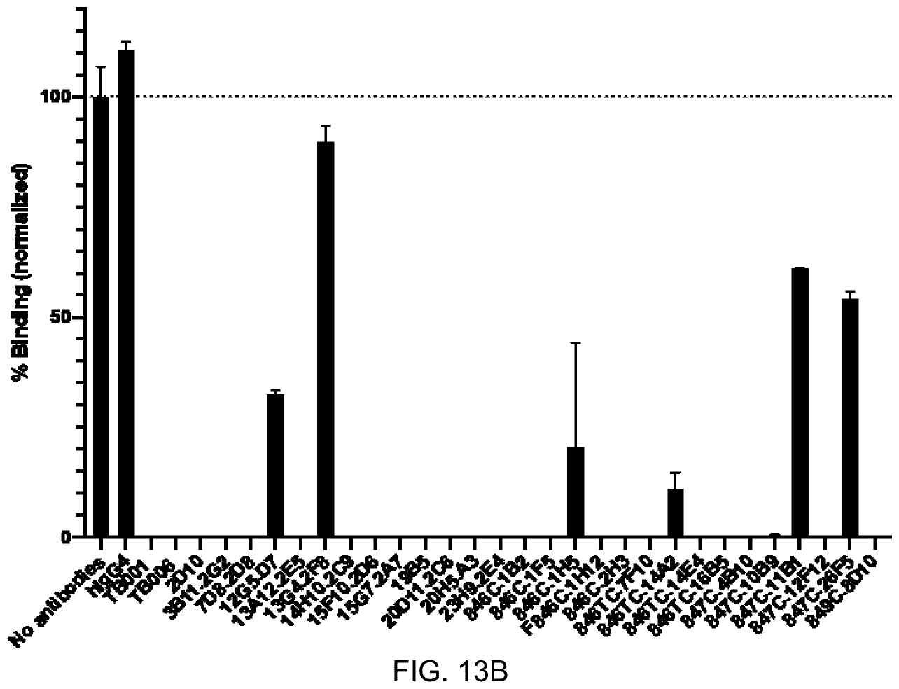

As depicted in FIG. 13A, TREM2 protein strongly bound GAL3-coated wells. TREM2 protein did not significantly bind to uncoated ELISA wells. Likewise, no significant binding signal was observed in wells with only GAL3 coating.

To identify Gal3-targeted antibodies with the ability to block the interaction of Gal3 and TREM2, purified Gal3 and TREM2 proteins were incubated in the presence (or absence) of various Gal3-targeted or control antibodies, or without antibody, and protein interaction was evaluated by ELISA.

Human GAL3 (TrueBinding, QCB200349) was diluted in PBS (Corning, 21-030-CM) to a concentration of 4 μg/mL and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Thereafter, the 2% BSA in PBST was discarded and 30 μL of control or anti-GAL3 antibodies at 20, 6.6, or 2.2 μg/mL was added to each well, followed by the addition of 30 μL of 3 or 4 μg/ml of recombinant human TREM2 protein (Sino Biological, 11084-H08H) in 2% BSA in PBST. The plate was incubated for an hour at room temperature with gentle rocking. Thereafter, the plate was washed three times with PBST. Anti-6× His tag antibody-HRP (Abcam, ab1269) was then added to the wells at 1:3000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm. Percent blockade of Gal3-TREM2 interaction was calculated as the fraction of signal obtained in the absence of antibody with the background signal subtracted.

As shown in FIG. 13B, anti-Gal3 antibodies with APP isoform 695 blocking activities differentially block the interaction of Gal3 and TREM2. Of the nineteen antibodies that blocked Gal3-APP695 >90%, eighteen of them also blocked Gal3-TREM2 >90% (2D10.2B2 [2D10], 20D11.2C6, 3B11.2G2, 20H5.A3, 846TC.14E4, 15G7.2A7, 14H10.2C9, 846C.2H3, TB001 (IMT001-4), 23H9.2E4, 846C.1F5, 15F10.2D6, 846TC.16B5, TB006 (IMT006-5 [4A11.H3L1]), 7D8.2D8, 846C.1B2, 849C.8D10, 19B5). Interestingly, two antibodies (847C.11B1, 847C.26F5) that displayed 87% blocking of Gal3-APP695, blocked Gal3-TREM2<47%. The six antibodies (849C.8D12, 849C.5H1, 6B3.2D3, 849C.3H2, 849C.1D2, 24D12.2H9) that did not block Gal3-APP695, were also poor blockers (0 to 30% blocking) of Gal3-TREM2 (not shown). Finally, 847C.14H4 and 846T.4C9 did not impact Gal3-TREM2 binding and were also poor blockers of Gal3-APP695 (not shown). Anti-Gal3 antibodies display differential ability to block GAL3 interaction with TREM2, therefore, Gal3 binding alone is not sufficient to disrupt the interaction of Gal3 and TREM2, and specific properties are required for this disrupting activity.

### Example 13: GAL3 Specifically Binds to Tau Aggregates

To evaluate the possibility that human galectin-3 (GAL3) could physically interact with human Tau aggregates, ELISA assessments with purified GAL3 and Tau aggregates were conducted. Human GAL3 protein (E. coli derived, TrueBinding, QCB200349; TrueBinding, QCB200352) was diluted in PBS (Corning, 21-030-CM) to concentrations of 4, 2, and 1 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Recombinant human Tau 412 (R&D Systems, SP-501) was aggregated as described below.

The concentration of recombinant Tau protein [tau-441 (2N4R) MW 45.9 kDa] was adjusted to 1 mg/ml by adding 10 mM Phosphate buffer pH 7.4. Aβ42 oligomers (7 μl, 0.3 mg/ml) were added to the sample (seeds) and mixed by pipetting for 1 min. The solution was continuously stirred during the aggregation time course using a stir bar at room temperature for 1 day. To investigate the aggregation state after 1 day, Tau peptide was probed with fibril specific conformation antibody OC (Sigma, AB 2286).

Tau aggregates were biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 4, 2, and 1 μg/ml of biotinylated recombinant Tau aggregates in 2% BSA in PBST was added to the wells. The plate was incubated for an hour at room temperature with gentle rocking. Thereafter, the plate was washed three times with PBST. Avidin HRP (Biolegend, 405103; 1:2000 dilution) was diluted in 2% BSA in PBST and then added to the wells. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

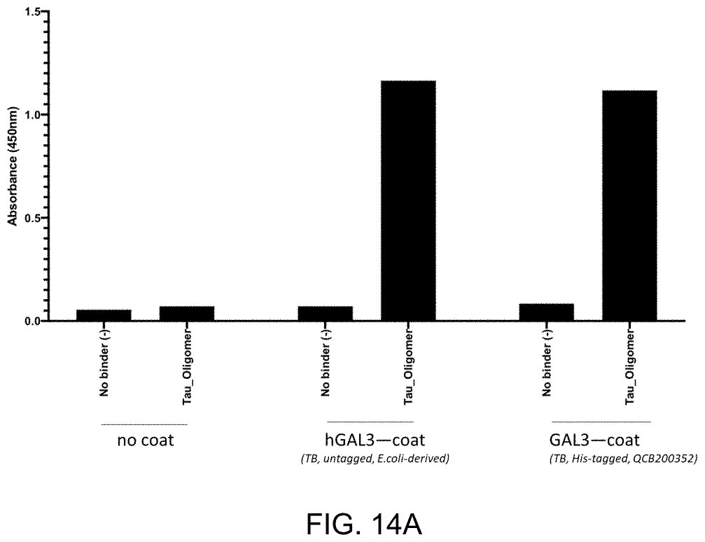

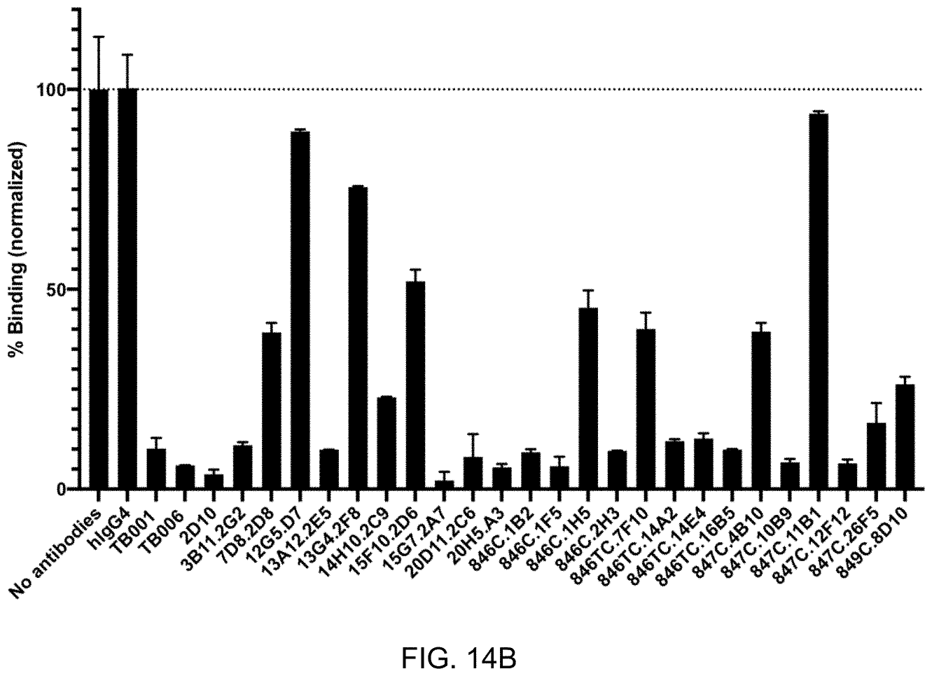

As depicted in FIG. 14A, Tau aggregates strongly bound GAL3-coated wells. Tau aggregates did not significantly bind to uncoated ELISA wells. Likewise, no significant binding signal was observed in wells with only GAL3 coating.

### Example 14: Identification of GAL3-Binding Antibodies with GAL3-Tau Aggregates Blocking Activity

To identify Gal3-targeted antibodies with the ability to block the interaction of Gal3 and Tau aggregates, purified Gal3 and Tau aggregates were incubated in the presence (or absence) of various Gal3-targeted or control antibodies, or without antibody, and protein interaction was evaluated by ELISA.

Human GAL3 (TrueBinding, QCB200352) was diluted in PBS (Corning, 21-030-CM) to a concentration of 2 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Recombinant human Tau 412 (R&D Systems, SP-501) was aggregated as described above. Tau aggregates were biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. Thereafter, the 2% BSA in PBST was discarded and 30 μl of control or anti-GAL3 antibodies at 20, 6.6, or 2.2 μg/ml was added to each well, followed by the addition of 30 μl of 4 μg/ml of biotinylated Tau aggregates in 2% BSA in PBST. The plate was incubated for an hour at room temperature with gentle rocking. Thereafter, the plate was washed three times with PBST. Avidin HRP (Biolegend, 405103) was then added to the wells at 1:2000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm. Percent blockade of Gal3-Tau aggregates interaction was calculated as the fraction of signal obtained in the absence of antibody with the background signal subtracted.

As shown in FIG. 14B, anti-GAL3 antibodies with APP isoform 695 blocking activities also block the interaction of GAL3 and Tau aggregates. Of all the antibodies tested, nine antibodies displayed blocking >90% (15G7.2A7, 2D10, 20H5.A3, 846.1F5, TB006, 84712F12, 847.10B9, 20D11.2C6, and 846.1B2) and eight more displayed blocking between 80 and up to 90% (846.2H3, 846T.16B5, 13A12.2E5, TB001, 3B11.2G2, 846T.14A2, 846T.14E4, and 847.26F5). Sixteen additional antibodies displayed <50% blocking of hGAL3-Tau aggregates (15F10.2D6, 13G4.2F8, 12G5.D7, 847.11B1, and data not shown). Anti-GAL3 antibodies display differential ability to block GAL3 interaction with Tau aggregates, therefore, Gal3 binding alone is not sufficient to disrupt the interaction of GAL3 and Tau aggregates, and specific properties were required for this disrupting activity.

### Example 15: GAL3 Specifically Binds to α-Synuclein Aggregates

To evaluate the possibility that human galectin-3 (GAL3) could physically interact with human α-Synuclein aggregates, ELISA assessments with purified GAL3 and α-Synuclein aggregates were conducted. Human GAL3 protein (E. coli derived, TrueBinding, QCB200349; TrueBinding, QCB200352) was diluted in PBS (Corning, 21-030-CM) to concentrations of 4, 2, and 1 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Recombinant human α-Synuclein (R&D Systems, SP-485) was aggregated as described.

Five (5) mg of the Alpha-synuclein peptide (MW 14 kDa) was diluted to final concentration 1 mg/ml by adding 10 mM sodium phosphate buffer, pH 7.4. The solution was continuously stirred during the aggregation time course using a stir bar at 37° C. for seven days. To investigate the aggregation state after 7 days, Alpha-synuclein peptide was probed with fibril specific conformation antibody OC (Sigma, AB 2286).

α-Synuclein aggregates were biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. After the plate was blocked, the 2% BSA in PBST was discarded and 4, 2, and 1 μg/ml of biotinylated recombinant α-Synuclein aggregates in 2% BSA in PBST was added to the wells. The plate was incubated for an hour at room temperature with gentle rocking. Thereafter, the plate was washed three times with PBST. Avidin HRP (Biolegend, 405103; 1:2000 dilution) was diluted in 2% BSA in PBST and then added to the wells. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

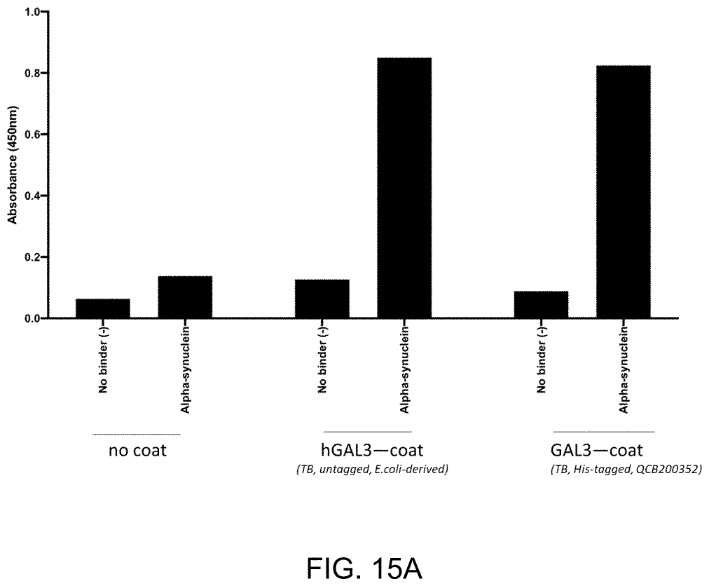

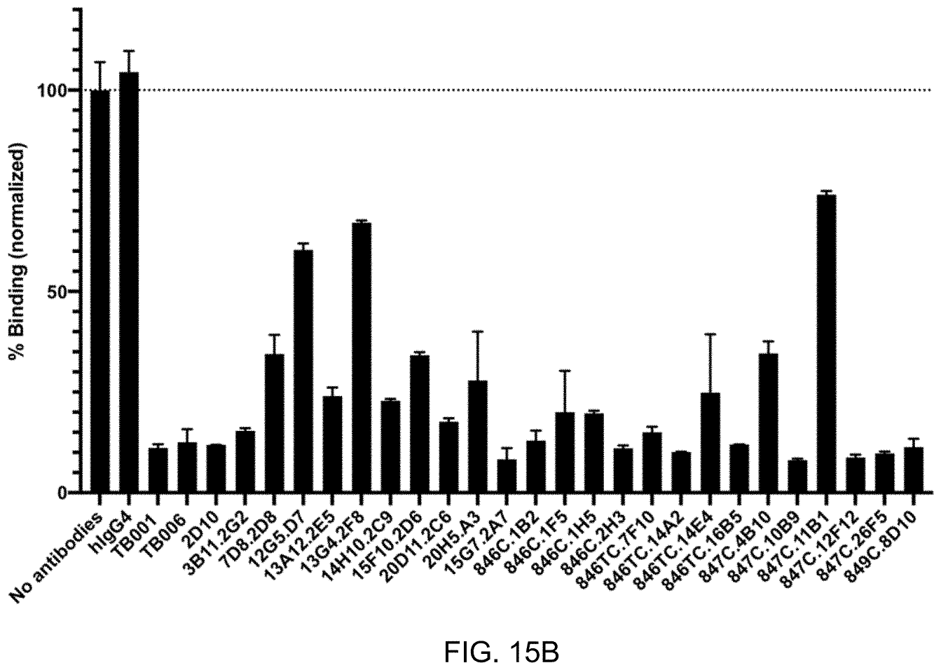

As depicted in FIG. 15A, α-Synuclein aggregates strongly bound GAL3-coated wells. α-Synuclein aggregates did not significantly bind to uncoated ELISA wells. Likewise, no significant binding signal was observed in wells with only GAL3 coating.

### Example 16: Identification of GAL3-Binding Antibodies with GAL3-α-Synuclein Aggregates Blocking Activity

To identify GAL3-targeted antibodies with the ability to block the interaction of Gal3 and α-Synuclein aggregates, purified GAL3 and α-Synuclein aggregates were incubated in the presence (or absence) of various GAL3-targeted or control antibodies, or without antibody, and protein interaction was evaluated by ELISA.

Human GAL3 (TrueBinding, QCB200352) was diluted in PBS (Corning, 21-030-CM) to a concentration of 4 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]) and then blocked for an hour with 2% BSA (EMD Millipore, 126609) in PBST at room temperature with gentle rocking. Recombinant human α-Synuclein (R&D Systems, SP-485) was aggregated as described above. α-Synuclein aggregates were biotinylated with EZ Link Sulfo-NHS-LC-Biotin (ThermoFisher Scientific, A39257) and desalted using a Zeba Spin Desalting Column (ThermoFisher Scientific, 89882), following the manufacturer's instructions. Thereafter, the 2% BSA in PBST was discarded and 30 μl of control or anti-GAL3 antibodies at 20, 6.6, or 2.2 μg/ml was added to each well, followed by the addition of 30 μl of 4 μg/ml of biotinylated u-Synuclein aggregates in 2% BSA in PBST. The plate was incubated for an hour at room temperature with gentle rocking. Thereafter, the plate was washed three times with PBST. Avidin HRP (Biolegend, 405103) was then added to the wells at 1:2000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm. Percent blockade of Gal3-α-Synuclein aggregates interaction was calculated as the fraction of signal obtained in the absence of antibody with the background signal subtracted.

As shown in FIG. 15B, anti-GAL3 antibodies with APP isoform 695 blocking activities also block the interaction of GAL3 and α-Synuclein aggregates. Of all the antibodies tested, three antibodies displayed blocking >90% (847.10B9, 15G7.2A7, and 84712F12) and ten more displayed blocking between 80 and 90% (846.2H3, TB001, 849.8D10, 2D10, 846T.16B5, TB006, 846.1B2, 846T.7F10, 3B11.2G2, and 20D11.2C6). Ten additional antibodies displayed <50% blocking of hGAL3-α-Synuclein aggregates (12G5.D7, 13G4.2F8, 847.11B1, and data not shown). Anti-GAL3 antibodies display differential ability to block GAL3 interaction with α-Synuclein aggregates, therefore, Gal3 binding alone is not sufficient to disrupt the interaction of GAL3 and α-Synuclein aggregates, and specific properties were required for this disrupting activity.

### Example 17: Antibodies Targeted to the N-Terminal Domain or TRD of Gal3 Penetrate Normal and Malignant Brain Tissue In Vivo

To validate ability of anti-GAL3 antibodies to penetrate tumor tissue, 7-week old Albino C57BL6 mice (The Jackson laboratory) were orthotopically transplanted with murine glioma cells GL261 that stably express firefly luciferase gene (GL261-LUC).

To generate GL261-LUC cells, GL261 murine glioblastoma cell line (DSMZ, no. ACC 802) was transduced with pLL-CMV-Luciferase-T2A-Puro Lenti-Labeler™ Pre-packaged Virus (SBI, catalog No. LL150VA-1) employing transduction reagent (TransDux MAX Transduction reagent, SBI, catalog No. LV860A-1) according to the manufacturer's instructions. The transduced cells were maintained in cell culture medium (90% DMEM, 10% FBS, 4 mM glutamine) containing 5 μg/mL of Puromycin for the duration of two weeks.

To generate biotinylated anti-GAL3 antibodies, biotinylation was performed with (EZ-Link™ NHS-PEG4-Biotin, No-Weigh™ Format, ThermoFisher Scientific, A39259) using the manufacturer's protocol. Briefly, NHS-PEG4-Biotin was dissolved in sterile water immediately before use and further diluted to 2 mM. One mg of each antibody in PBS was then transferred to clean tubes and mixed with 3.33 μL of biotin for a 1:1 molar ratio. The mixtures were incubated at room temperature for 30 minutes. For some samples, unreacted biotin was removed using a concentrator (Pierce™ Protein Concentrators PES, 10K MWCO, 0.5 mL, ThermoFisher Scientific, 88513) by repeatedly concentrating and diluting biotinylated samples with DPBS for 3 times. For other samples, unreacted biotin was removed by spinning and collecting fractions containing only the biotinylated antibodies using desalting columns (Zeba™ Spin Desalting Columns, 7K MWCO, 2 mL, ThermoFisher Scientific, 89890). The final concentrations of biotinylated samples were calculated based on their UV absorption at 280 nm.

GL261-LUC (5×10⁵) were transplanted into the forebrain of the recipient animals (1 mm posterior of bregma, 2 mm lateral of midline and 3.5 mm deep, employing a stereotaxic frame (Stoelting, 51725D)). Implantation was confirmed using imaging of tumor luminescence. Briefly, luminescent images were obtained 10 min after injection with XenoLight D-Luciferin-K+Salt Bioluminescent Substrate (Perkin Elmer, cat #122799, prepared according to manufacturer protocol) using an IVIS-Spectrum CT Imaging System (PerkinElmer). When bioluminescence flux detected in brains of injected animals reached 10⁶ photons/second, the animals were injected i.v. with biotinylated anti-GAL3 antibodies (10 mg/kg). Four days later, the animals were sacrificed, and blood and brain tissue samples were collected.

To generate plasma samples, blood was collected in K2-EDTA tubes, centrifuged at 13000 RPM for 3 min and transferred into fresh tubes. To generate brain tissue lysates, malignant and normal brain samples were harvested and frozen in liquid nitrogen. Tissue fragments were homogenized in lysis buffer (0.2% Tween-20, 1× Proteinase cocktail [Sigma, P8340] in PBS) in a ratio of 1 to 10 by employing TissueLyser II (Qiagen, Frequency: 30.0 l/s; for 30 sec). Samples were incubated at 4° C. overnight for protein extraction, and then spun down at 10000 rpm for 3 min at 4° C. to collect supernatants.

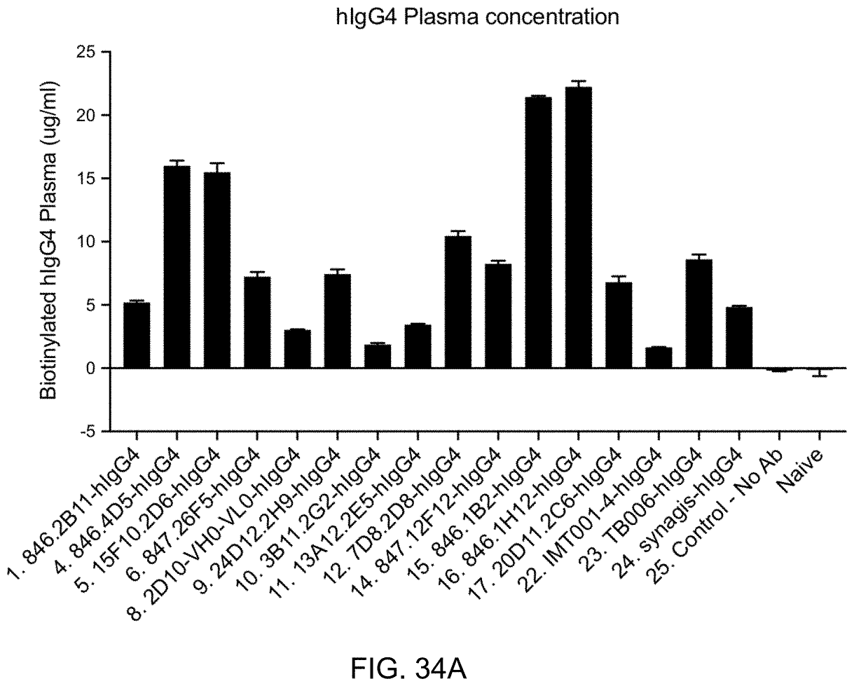

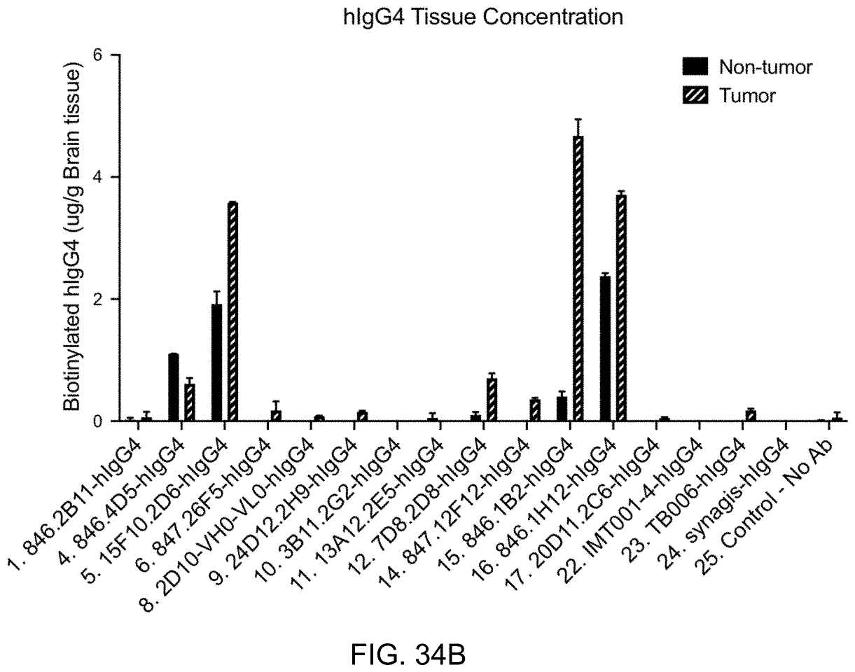

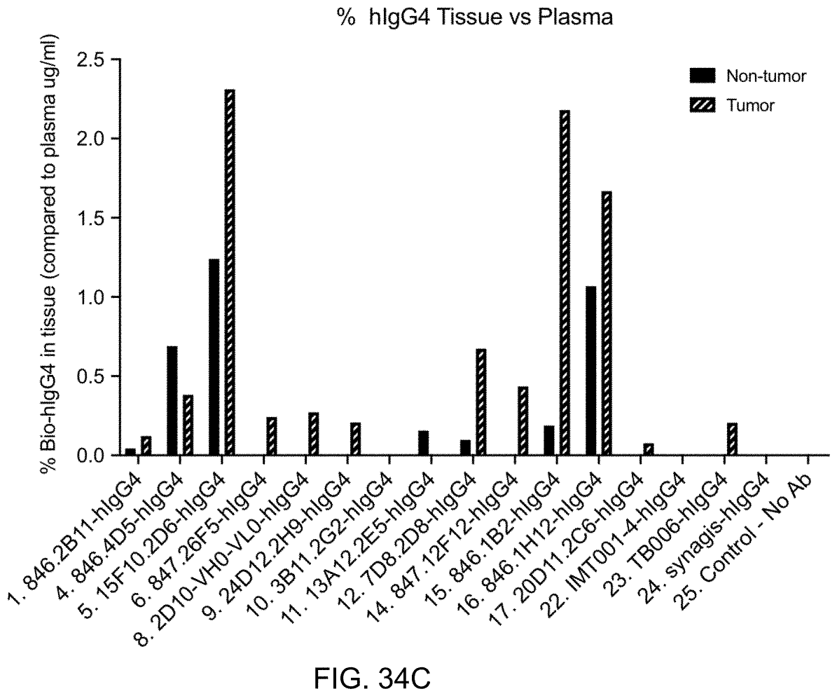

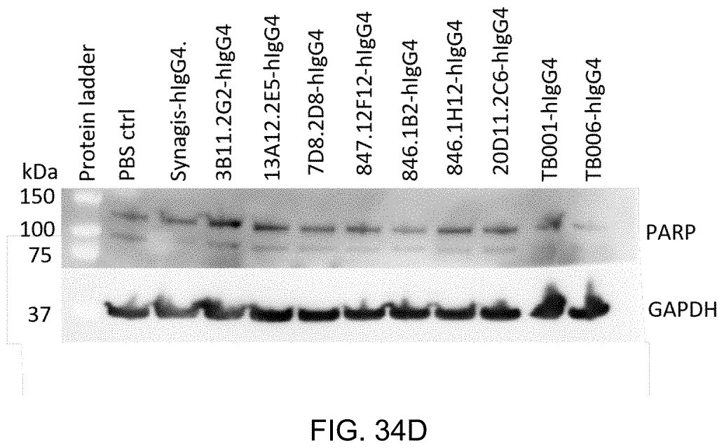

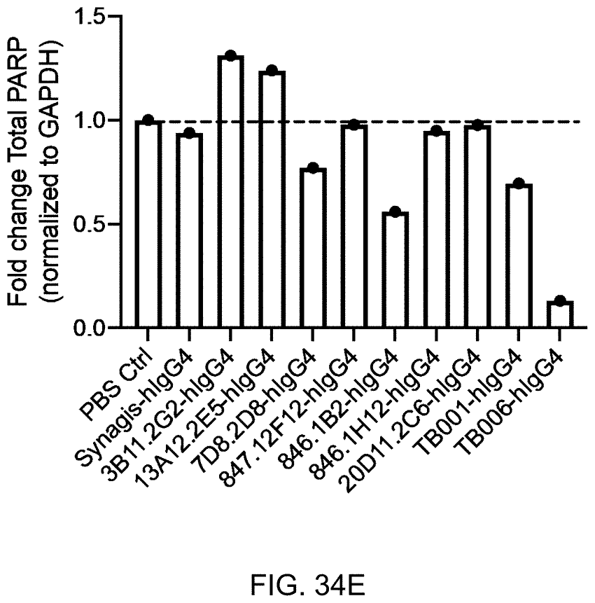

To determine the concentration of biotinylated antibodies in plasma and brain samples, the levels of biotinylated anti-GAL3 antibodies were determined by ELISA-based assay. Briefly, the plates were coated overnight at 4° C. with 50 ng/well RM120 (Anti-Human IgG4 Rabbit mAb, RevMAb Biosciences). Twenty-four hours after incubation, the plates were blocked with PBST containing 2% BSA for 1 hr at room temperature. Each brain tissue lysate was diluted ten-fold and each plasma sample was diluted fifty-fold in PBST containing 2% BSA and added to the plate for 2 hrs. Following three washes with PBS, an HRP-conjugated Avidin (Biolegend, 405103) was added to the plates. Following additional incubation for one-hour, the plates were washed three times with PBS and developed with TMB. The reaction was stopped by addition of 1M HCL. As presented in FIG. 34A-C, anti-GAL3 antibodies belonging to various bins demonstrated differential activity in penetrating the blood brain barrier (BBB) with antibodies belonging to bins 3, 8, 17 and 24 (e.g. 846.4D5, 15F10.2D6, 846.1B2, 846.1H12) exhibiting higher penetrance than the rest of the tested clones.

To determine the biological activity of antibodies in brain tissue, the levels of antibody-induced apoptosis was analyzed in tumor samples. Brain tumor lysates were probed for the marker of cell death PARP on a Western blot. Briefly, 1× sample buffer (Thermo-Fisher, NP0007) and 1× reducing agent (Thermo-Fisher, NP0009) was added to 20 μg of lysate, then heated to 72° C. for 10 min. Protein ladder (BioRad, 161-0374) and sample was loaded onto a 4-12% NuPage Bis-Tris gel (Thermo-Fisher, NP0322) and run in 1×MES buffer (Thermo-Fisher, NP0002) at 180V for 55 min with 500 μl antioxidant (Thermo-Fisher, NP0005) added to the inner chamber. Protein was transferred for 7 minutes from the gel to nitrocellulose membrane in the iBlot Gel transfer stacks (Thermo-Fisher, IB23001) using the iBlot II system (Thermo-Fisher, IB21001). Membrane was washed in distilled water and blocked for 1 hour with 5% bovine serum (BSA) (Sigma, A9418) in TBS and 0.1% Tween-20 (Sigma, P2287) (TBS-T). The nitrocellulose membrane was then washed with TBS-T for ten minutes three times. Primary antibodies to the apoptosis marker PARP (Cell Signal Technology, 9542) and a protein loading control GAPDH (Cell Signal Technology, 5174) were diluted 1:1000 into 5% BSA in TBS-T and incubated overnight. The nitrocellulose membrane was then washed with TBS-T for ten minutes three times. A secondary goat anti-rabbit-HRP antibody (Abcam, ab97051) was diluted 1:5000 in 5% BSA TBS-T and incubated with the membrane for 1 hour at room temperature, then washed with TBS-T for ten minutes three times. Finally, protein was detected by adding Supersignal West Pico Chemiluminescent substrate (Thermo-Fisher, 34579) to the membrane for 1 minute and imaged using Azure Biosystem c400. GAPDH levels were used as loading control (FIG. 34D).

Images were imported in ImageJ software and the signal intensity in each sample was quantified as a measure of protein quantity. To account for any differences in protein loading, signal intensity of PARP was divided by signal intensity of GAPDH for that sample. Briefly, intensity of PARP and GAPDH in the control sample was set as “1”, then the intensity of all other samples was divided by that value to get a measure relative to control. Finally, each sample's relative value for PARP was divided by its relative value for GAPDH. Normalized values were then compared to control to determine whether full PARP levels in tumors were changed by Ab treatment. The reduced levels of PARP (<1) are indicative of the increased apoptotic activity in tumor samples (FIG. 34E).

As seen in FIG. 34D-E, antibodies 7D8.2D8, 846.1B2, TB001 and TB006 reduced levels of PARP when compared to control samples, signifying increased biological activity (apoptosis) in the tumor.

### Example 18. Anti-Gal3 Antibodies can be Used to Treat Neurodegenerative Diseases in Humans

Patients present with a neurodegenerative disease. Some non-limiting examples of neurodegenerative diseases are inflammation, encephalitis, Alzheimer's disease, Parkinson's disease, Huntington's disease, traumatic brain injury, spinal injury, multiple sclerosis, amyotrophic lateral sclerosis, olfactory dysfunction, aphasia, Bell's palsy, transmissible spongiform encephalopathy, Creutzfeldt-Jakob disease, fatal familial insomnia, epilepsy, seizures, neurodevelopment, Tourette's syndrome, neuroinfectious disorders, meningitis, encephalitis, bovine spongiform encephalopathy, West Nile virus encephalitis, Neuro-AIDS, fragile X syndrome, Guillain-Barre syndrome, metastases to the brain, brain cancer, or any combination thereof. One or more anti-Gal3 antibodies or binding fragments thereof disclosed herein are administered to the patients enterally, orally, intranasally, parenterally, intracranially, subcutaneously, intramuscularly, intradermally, or intravenously.

The anti-Gal3 antibodies or binding fragments thereof are administered as doses at an amount of 1 ng (or in the alternative: 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans). The doses are administered every 1 day (or in the alternative: every 2, 3, 4, 5, 6, 7, 8, 9, 10, 11, 12, 24, 36, or 48 days or weeks or any time within a range defined by any two of the aforementioned times).

An improvement of the neurodegenerative disease or symptoms associated with the neurodegenerative disease is observed in the patients following administration of the anti-Gal3 antibodies or binding fragments thereof.

### Example 19. Anti-Gal3 Antibodies can be Used to Treat Proteopathies in Humans

Patients present with a proteopathy. In some cases, the proteopathy is hereditary. Some non-limiting examples of proteopathies are Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof. One or more anti-Gal3 antibodies or binding fragments thereof are administered to the patients enterally, orally, intranasally, parenterally, intracranially, subcutaneously, intramuscularly, intradermally, or intravenously.

The anti-Gal3 antibodies or binding fragments thereof are administered as doses at an amount of 1 ng (or in the alternative: 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans). The doses are administered every 1 day (or in the alternative: every 2, 3, 4, 5, 6, 7, 8, 9, 10, 11, 12, 24, 36, or 48 days or weeks or any time within a range defined by any two of the aforementioned times).

An improvement of the proteopathy or symptoms associated with the proteopathy is observed in the patients following administration of the anti-Gal3 antibodies or binding fragments thereof.

### Example 20. Anti-Gal3 Antibodies can be Used to Treat Alzheimer's Disease in Humans

Patients present with Alzheimer's disease. One or more anti-Gal3 antibodies or binding fragments thereof are administered to the patients enterally, orally, intranasally, parenterally, intracranially, subcutaneously, intramuscularly, intradermally, or intravenously.

The anti-Gal3 antibodies or binding fragments thereof are administered as doses at an amount of 1 ng (or in the alternative: 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans). The doses are administered every 1 day (or in the alternative: every 2, 3, 4, 5, 6, 7, 8, 9, 10, 11, 12, 24, 36, or 48 days or weeks or any time within a range defined by any two of the aforementioned times).

An improvement of the Alzheimer's disease or symptoms associated with Alzheimer's disease is observed in the patients following administration of the anti-Gal3 antibodies or binding fragments thereof. Administration of the anti-Gal3 antibodies or binding fragments may be performed in conjunction with another therapy for Alzheimer's disease, for example, a cholinesterase inhibitor (e.g. tacrine, rivastigmine, galantamine, donepezil), an NMDA receptor antagonist (e.g. memantine), or both.

### Example 21. Assessment of Gal3 Binding to TGF-Beta Receptor Family Members

To evaluate the possibility that galectin-3 (GAL3) could physically interact with TGF-beta receptor family members, ELISA assessments with purified GAL3 and various TGF-beta receptor proteins were conducted. Briefly, human galectin-3 protein (R&D Systems, 1154-GA/CF or 1154-GA) was diluted in PBS (Corning, 21-030-CM) to a concentration of 3 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]). The plate was then blocked for an hour with 2% BSA (EMD Millipore, 126609) in PBST at room temperature with gentle rocking. Thereafter, the 2% BSA in PBST was discarded and 50 ul of 3 μg/ml of recombinant human TGF-beta RI/ALK-5 Fc chimera protein (R&D Systems, 3025-BR) or 3 μg/ml of recombinant human TGF-beta RI/ALK-5 Fc chimera protein (R&D Systems, 341-BR), or 3 μg/ml of recombinant human TGF-beta RIII (R&D Systems, 242-R3-100) in 2% BSA in PBST was added each well. For combinations of TGF-beta receptors, equivalent volumes of 3 μg/ml protein were mixed and 50 ul of this mixture was added per well. The plate was incubated for an hour at room temperature with gentle rocking. Thereafter, the plate was washed three times with PBST. Peroxidase AffiniPure F(ab′)₂ Fragment Goat Anti-Human IgG (H+L) polyclonal antibody (Jackson ImmunoResearch, 109-036-003) was then added to the wells at 1:4000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

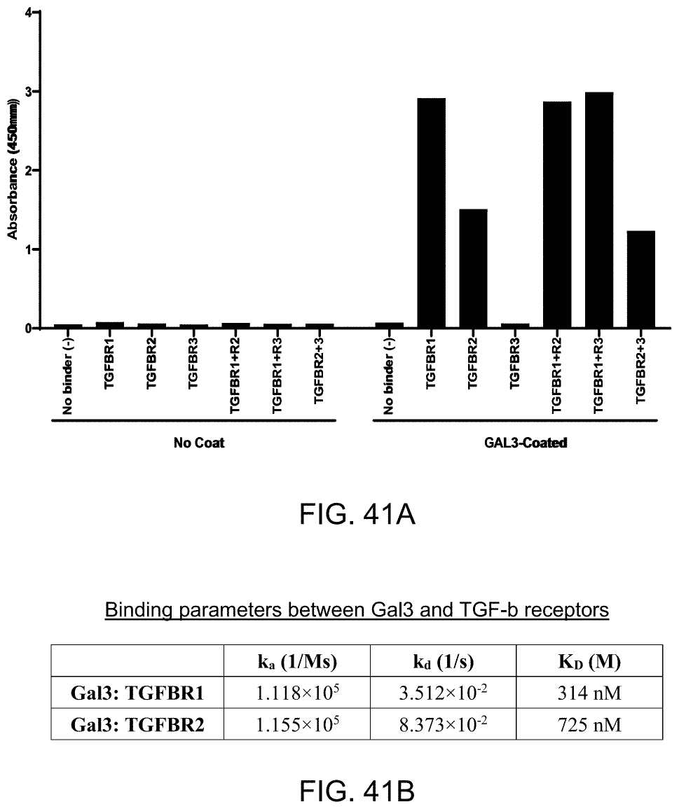

As depicted in FIG. 41A, no significant binding of TGFβ RI, TGFβ R2, or TGFP R3 was observed to plates without GAL3 coating, similarly, combinations of TGFβ RI and TGFβ R2, TGFβ RI and TGFβ R3, and TGFβ R2 and TGFβ R3 failed to bind uncoated ELISA wells. In contrast, TGFβ RI and TGFβ R2 strongly bound GAL3-coated wells, whereas TGFβ R3 did not show binding activity. Mixtures of TGFβ RI and TGFβ R2, TGFβ RI and TGFβ R3, and TGFβ R2 and TGFβ R3 exhibited significant binding to GAL3-coated wells, but no additional binding activity was observed relative to TGFβ RI or TGFβ R2 alone.

To further explore this observation, the kinetics of GAL3 assembly and disassembly with TGFβ R1 or TGFβ R2 were evaluated by surface plasmon resonance. Briefly, kinetics experiment was performed on Caterra LSA at 25° C. A HC30M chip was immobilized with recombinant Protein A/G.

Ligand proteins, recombinant human TGF-beta RI/Fc (R&D system #3025-BR) and recombinant human TGF-beta sRII/Fc (R&D system #341-BR) 5 μg/ml each were loaded onto different spots among the 384-point array.

The analyte recombinant human Gal3 at concentrations of 0, 2, 4, 8, 16, 32, 64 and 128 nM, one concentration at a time, was injected to the whole array. The complex was allowed to associate and dissociate for 300s and 240s, respectively. Eight cycles of kinetics were done sequentially for the dilution series. The kinetics constants were calculated by the software NextGenKIT and depicted in FIG. 41B.

### Example 22. Gal3-Targeted Monoclonal Antibodies Block Binding of Gal3 to TGFBR1

To identify Gal3-binding antibodies with the capacity to block the assembly of Gal3 and TGF-beta R1, purified Gal3 and TGF-beta R1 proteins were incubated in the presence of a panel of Gal3-targeted monoclonal antibodies, with non-specific control antibodies, or without antibody, and protein interaction was evaluated by ELISA. Briefly, human galectin-3 protein was diluted in PBS to a concentration of 3 μg/ml and added to the wells of a 96-well ELISA plate. After incubating the plate at 4° C. overnight, the plate was washed three times with PBST. The plate was then blocked for an hour with 2% BSA in PBST at room temperature with gentle rocking. Thereafter, the 2% BSA in PBST was discarded and 25 ul of control or anti-GAL3 antibodies at 20 μg/ml were added to each well, followed by the addition of 25 μl of 6 μg/ml of recombinant human TGF-beta RI/ALK-5 Fc chimera protein (R&D Systems, 3025-BR) to each well. Plate was incubated for an hour at room temperature with gentle rocking. Thereafter, the plate was washed three times with PBST. Peroxidase AffiniPure F(ab′)₂ Fragment Goat Anti-Human IgG (H+L) polyclonal antibody was then added to the wells at 1:4000 dilution in 2% BSA in PBST. The plate was incubated at room temperature for an hour with gentle rocking and then washed three times with PBST. TMB substrate was then added to each well. The reaction was stopped with 1M HCl and read using a plate reader at absorbance of 450 nm.

As depicted in FIG. 42, GAL3-targeted antibodies exhibited a range of inhibitory activity against the binding of GAL3 to TGFβ RI. Several antibodies exhibited no significant inhibitory activity, including 7D8.2D8, and 24D12.2H9. Other GAL3-targeted antibodies inhibited GAL3 assembly with TGFβ RI by 10%-25%, including 4G2.2G6 and 15F10.2D6. Other GAL3-targeted antibodies inhibited GAL3 assembly with TGFβ RI by 25%-50%, including 3B11.2G2, 9H2.2H10, 13A12.2E5, 13G4.2F8, 13H12.2F8, and 14H10.2C9. Other GAL3-targeted antibodies inhibited GAL3 assembly with TGFβ RI by 50%-75%, including 4A11.2B5, 12G5.D7, 15G7.2A7, 19B5.2E6, 20D11.2C6, 23H9.2E4, and mTB001 (mIMT001, murine IMT001). Still other GAL3-targeted antibodies inhibited GAL3 assembly with TGFβ RI by 85% or greater, including 2D10.2B2, 6H6.2D6, and 20H5.Aβ. Collectively, these observations reveal that distinct classes of GAL3-targeted antibodies exist with varying ability to interfere with the association of GAL3 with TGFβ RI.

### Example 23. Gal3-Targeted Monoclonal Antibodies Block Binding of Gal3 to TGFBR2

Human Galectin-3 (R&D Systems, 8259-GA) protein was diluted in PBS to a concentration of 4 μg/ml and 40 μl added to the wells of a 96-well ELISA plate. After incubating the plate at 4° C. overnight, the plate was washed three times with 300 μl PBST. The plate was then blocked for an hour with 200 μl of 2% BSA in PBST at room temperature with gentle rocking. During blocking, TGFβR2 (R&D Systems, 341-BR, OR1619011) was biotinylated with Sulfo-NHS-LC-Biotin following Thermo Scientific instructions. After biotinylation, proteins were desalted using Zeba Spin Desalting Columns following Thermo Scientific instructions. Thereafter, the 2% BSA in PBST was discarded and 4A11.H3L1, TB001 (IMT001), or Synagis hIgG4 (3-fold dilutions beginning at 180 μg/ml) in 2% BSA in PBST was added to the wells. Afterwards, 4 μg/ml of TGFβR2 in 2% BSA in PBST was added to the antibodies in the wells in a 1:1 ratio (30 μl: 30 μl). The plate was incubated for an hour at room temperature with gentle rocking. Thereafter, the plate was washed three times with 300 μl PBST. Avidin HRP (1:2000) was diluted in 2% BSA in PBST and then 40 μl added to the wells. The plate was incubated at room temperature for an hour with gentle rocking and then washed three times with 300 μl PBST. TMB substrate (50 μl) was then added to each well. The reaction was stopped with 1N HCl (25 μl) and read using a plate reader at absorbance of 450 nm. GAL3-targeted antibodies efficiently inhibited the binding of GAL3 to TGFP R2. 4A11.H3L1 blocks the Gal3::TGFBR2 interaction and displays an IC₅₀ of 11.5 nM. TB001 (IMT001) blocks the Gal3::TGFBR2 interaction and displays an IC₅₀ of 12.8 nM.

### Example 24. Gal3-Targeted Monoclonal Antibodies with TGFBR BLOCKING ACTIVITY INHIBIT TGF-b Induction of TGF-b Target Genes

To evaluate the functional consequence of interference of GAL3 binding to TGFβ RI, TGFβ RI expressing human hepatic stellate LX2 cells, were incubated with purified GAL3-targeted antibodies that block assembly of GAL3 and TGFβ RI, 19B5.2E6, mIMT001, and 4A11.2B5, or with a purified GAL3-targeted antibody that does not block assembly of GAL3 and TGFβ RI, 24D12.2H9, or with a purified non-targeted IgG2a isotype control antibody, and cells were evaluated for responsiveness to TGFβ induction of TGFβ target genes. Briefly, LX2 cells (Millipore, Cat #SCC064) were plated in 60 mm diameter dishes, cultured to 70% confluence, and were serum starved for 24 hours prior to addition of 2 ng/ml TGF-β (R&D Systems 240-B-002) and various anti-Galectin-3 antibody or control antibody treatment (10 μg/ml) for 24 hrs. Total RNA from cells was extracted with Quick-RNA MiniPrep kits (Zymo Research, Cat #R1054) according to the manufacturer's instruction. RNA was quantified via spectrophotometry using a ThermoFisher Nanodrop 8000, and 0.5 ug total RNA was reverse transcribed to cDNA using the Iscript Reverse Transcription Supermix kits (Bio-Rad, Cat #1708841). The reaction was performed with a 5-minute incubation at 25° C., followed by a 20-minute incubation at 46° C., and finally a 1-minute incubation at 95° C. Quantitative real-time PCR was performed with Roche LightCycler 96 system. 3 ul cDNA was used in each PCR reaction. The housekeeping gene ATPF1 was used as a reference gene for normalization and a blank H2O sample was used as a negative control. The primer pair sequences for alpha smooth muscle actin (uSMA) were:

(SEQ ID NO: 572)

5′ -AGCCAAGCACTGTCAGGAATC- 3′
and

(SEQ ID NO: 573)

5′ -GGGCAACACGAAGCTCATTG- 3′;
for collagen type 1 alpha 1 (Co1A1):

(SEQ ID NO: 574)

5′ CAAAGAAGGCGGCAAAGGTC- 3′
and

(SEQ ID NO: 575)

5′ -CCCTCACGTCCAGATTCACC- 3′;
for connective tissue growth factor (CTGF):

(SEQ ID NO: 576)

5′ TCCACCCGGGTTACCAATG- 3′
and

(SEQ ID NO: 577)

5′ -CCGGGACAGTTGTAATGGCA- 3′;
for fibronectin (FN):

(SEQ ID NO: 578)

5′ CACCTCTGTGCAGACCACAT- 3′
and

(SEQ ID NO: 579)

5′ -ACGTCCTGCCATTGTAGGTG- 3′;
for Gal-3:

(SEQ ID NO: 580)

5′ ACAATTCTGGGCACGGTGAA- 3′
and

(SEQ ID NO: 581)

5′ -CGTGGGTTAAAGTGGAAGGC- 3′.

The PCR reaction was catalyzed by PerfeCTa SYBR Green SuperMix (Quanta, Cat #95054-100) and SYBR Green fluorescent signal was measured during each of the 40 cycles, and the number of cycles required to surpass a threshold of detection (Ct) was enumerated. The Ct of each specific reaction was normalized to that obtained with ATPF1, and normalized Cts were further referenced to control-treated samples to establish relative expression levels of each gene.

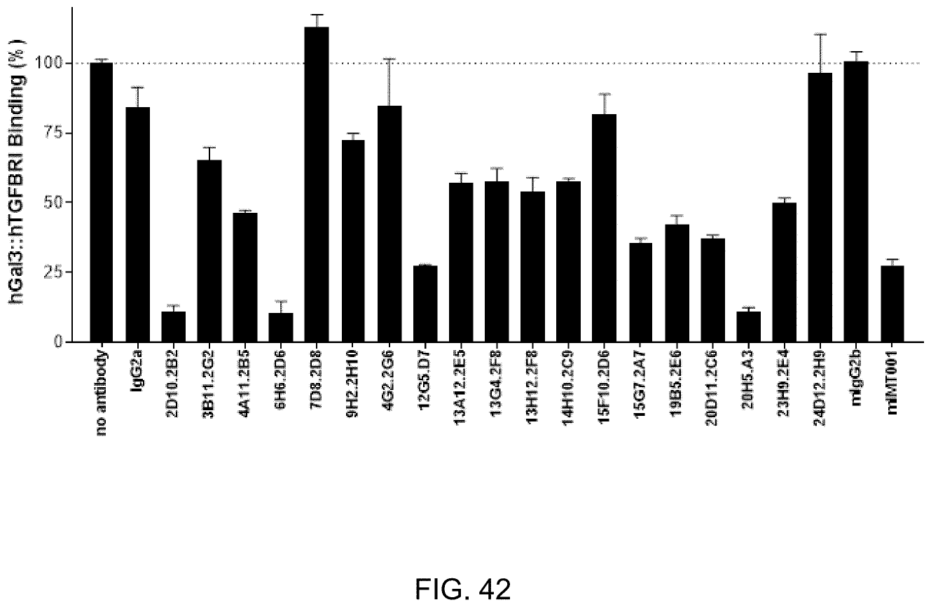

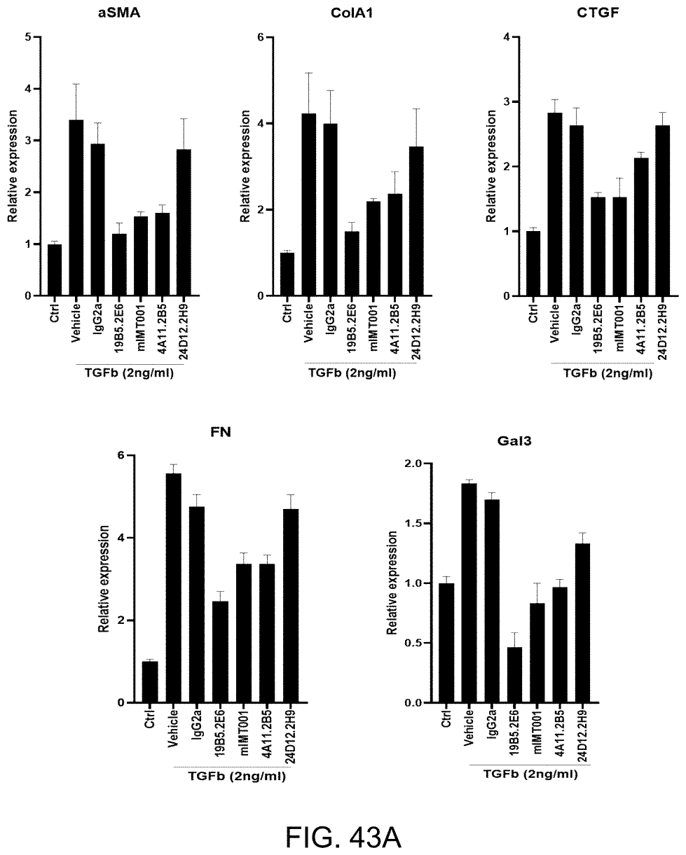

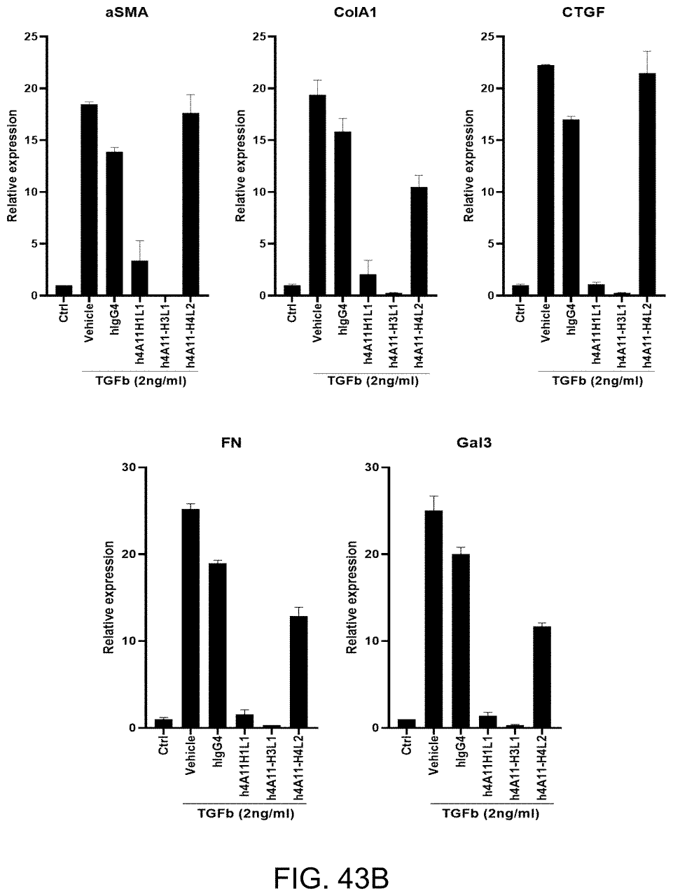

As depicted in FIG. 43A, LX2 cells stimulated with TGFβ exhibited induction of TGFP target genes alpha smooth muscle actin (a-SMA), collagen type 1 alpha 1 (ColA1), connective tissue growth factor (CTGF), fibronectin (FN), and GAL3 when treated with vehicle or isotype control antibodies. Conversely, TGFβ induction of a-SMA was blunted by 92%, 78%, and 75% by GAL3-TGFβ R blocking antibodies 19B5.2E6, mIMT001, and 4A11.2B5. Significantly, the GAL3-targeted antibody lacking GAL3-TGFβ R blocking activity, 24D12.2H9, failed to show any significant inhibitory effect on TGFβ induction of a-SMA. Similar trends were observed with TGFβ induction of ColA1, with 85%, 63%, and 58% reductions in induction by 19B5.2E6, mIMT001, and 4A11.2B5, respectively, and no significant impact of 24D12.2H9. This effect was also observed with CTGF, FN, and GAL3, all of which exhibited significant inhibition of TGFβ induction in the presence of GAL3-TGFβ R1 blocking antibodies, but no reduction in activity with isotype control or non-blocking GAL3 antibodies. These data indicate that GAL3-targeted antibodies have the capacity to inhibit TGFβ induction of TGFβ target genes, and that this activity is limited to those antibodies with the capacity to block GAL3 and TGFβ R binding.

To further evaluate the activity of GAL3-targeted antibodies with potential as human therapeutics, a series of humanized antibody derived from the 4A11.2B5 murine antibody were evaluated for the ability to inhibit TGFβ induction of TGFβ target genes in LX2, depicted in FIG. 43B. Remarkably, two of the humanized variants, 4A11.H1L1 (IMT006b, IMT006-1), and 4A11.H3L1 (IMT006a, IMT006-5) showed the ability to completely inhibit TGFP induction of TGFP target genes a-SMA, ColA1, CTGF, FN, and Gal3, whereas the humanized variant 4A11.H4L2 (IMT006c, IMT006-8) exhibited greatly reduced or no inhibitory activity. These observations indicate that humanized antibodies targeting GAL3 can effectively inhibit TGFP induction of TGFP target genes.

### Example 25. Assessment of Gal3 Binding to Tumor Cell Surface Receptors

To evaluate the possibility that galectin-3 could physically interact with tumor cell surface receptors, ELISA assessments with purified GAL3 and various tumor cell surface receptor proteins were conducted. Briefly, human galectin-3 protein (R&D Systems, 8259-GA or Acro Biosystems, GA3-H5129) was diluted in PBS (Corning, 21-030-CM) to a concentration of 2 or 3 μg/ml and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]). The plate was then blocked for an hour with 2% BSA (EMD Millipore, 126609) in PBST at room temperature with gentle rocking. Thereafter, the 2% BSA in PBST was discarded and 2 or 3 μg/ml of various recombinant human tumor cell surface proteins in 2% BSA in PBST were added to the wells. The human tumor cell surface proteins used include recombinant human EGFR (R&D Systems, 344-ER), VEGFR1 (R&D Systems, 321-FL/CF), VEGFR2 (R&D Systems, 357-KD/CF), VEGFR3 (R&D Systems, 349-F4), PDGFRa (R&D Systems, 6765-PR), PDGFRb (R&D Systems, 385-PR/CF), Her2 (R&D Systems, 1129-ER), FGFR1 alpha IIIb (R&D Systems, 655-FR), FGFR2 alpha IIIb (R&D Systems, 663-FR), hFGFR1 alpha IIIc (R&D Systems, 658-FR), hFGFR2 alpha IIIc (R&D Systems, 712-FR), hFGFR3 IIIc (R&D Systems, 766-FR), hFGFR4 (R&D Systems, 685-FR), HGFR (cMet) (R&D Systems, 8614-MT), TNF sRI (R&D Systems, 372-RI/CF), hCTLA4 (R&D Systems, 7268-CT), hCD47 (R&D Systems, 4670-CD), hPDL1 (R&D Systems 156-B7).

The plate was incubated for an hour at room temperature with gentle rocking. Thereafter, the plate was washed three times with PBST. Peroxidase AffiniPure F(ab′)₂ Fragment Goat Anti-Human IgG (H+L) polyclonal antibody (Jackson ImmunoResearch, 109-036-003) was then added to the wells at 1:4000 dilution in 2% BSA in PBST. The plate was incubated at room temperature for an hour with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

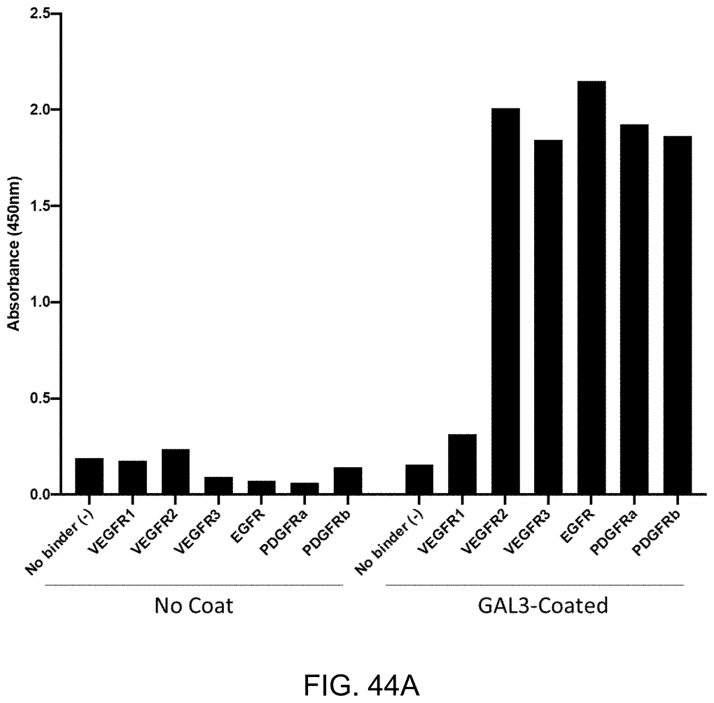

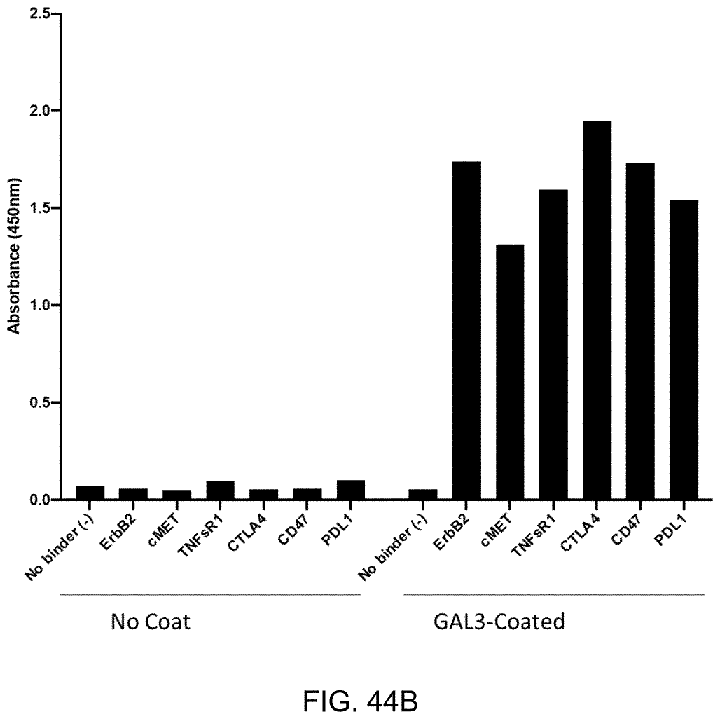

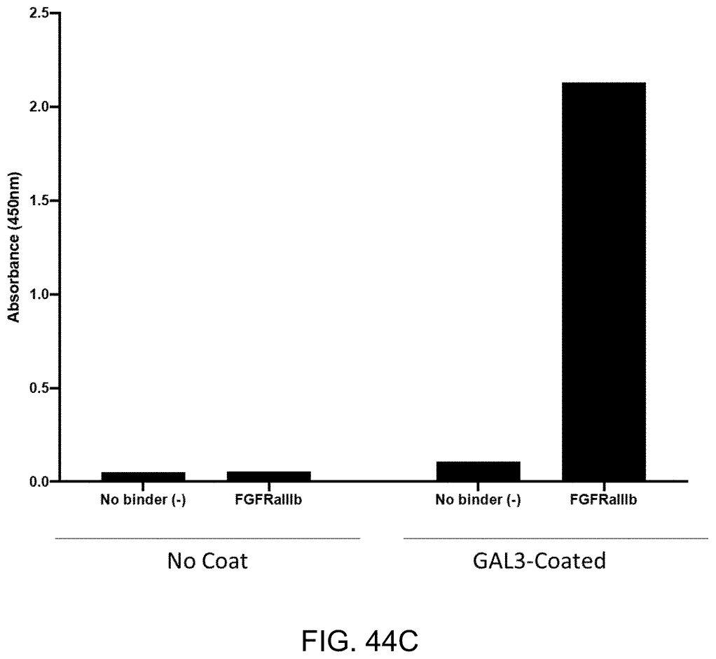

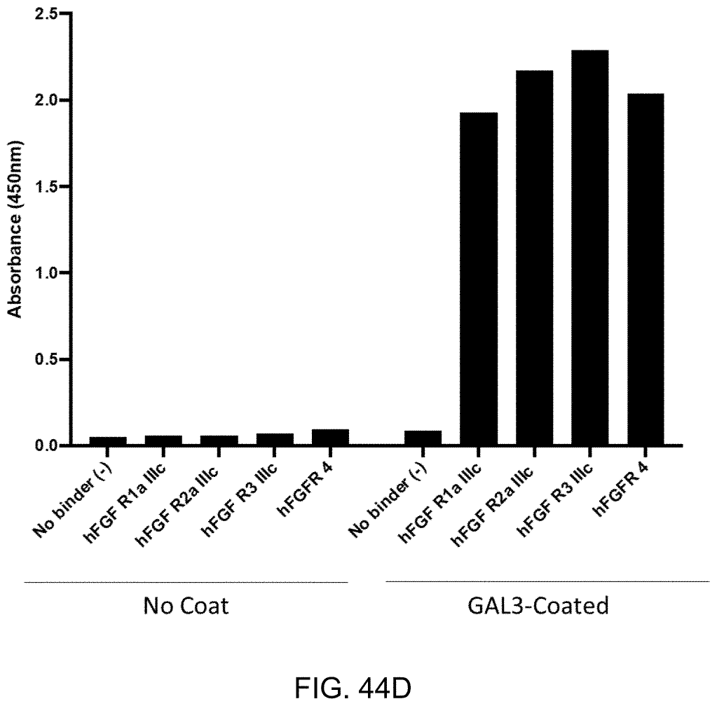

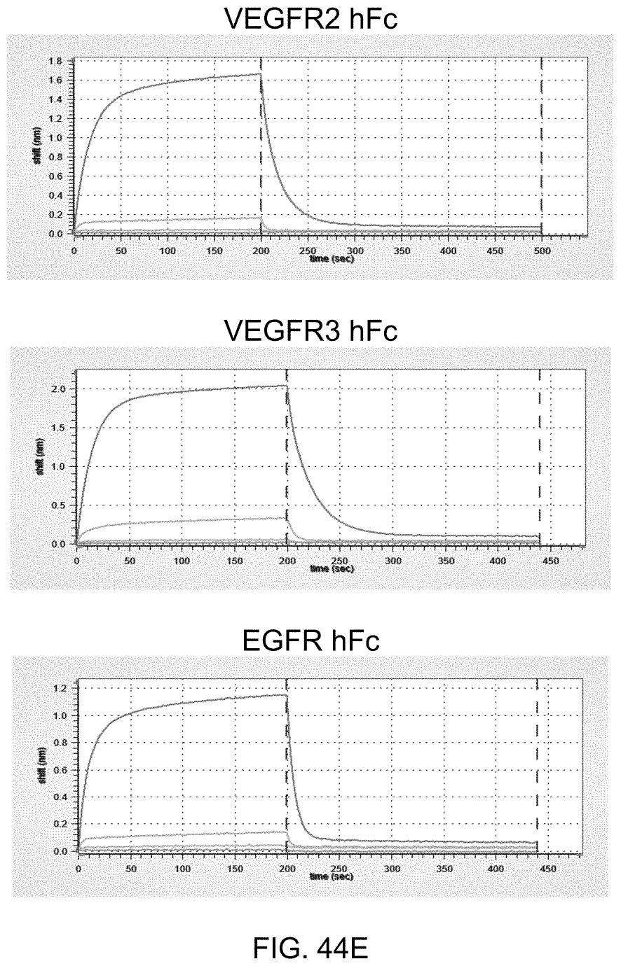

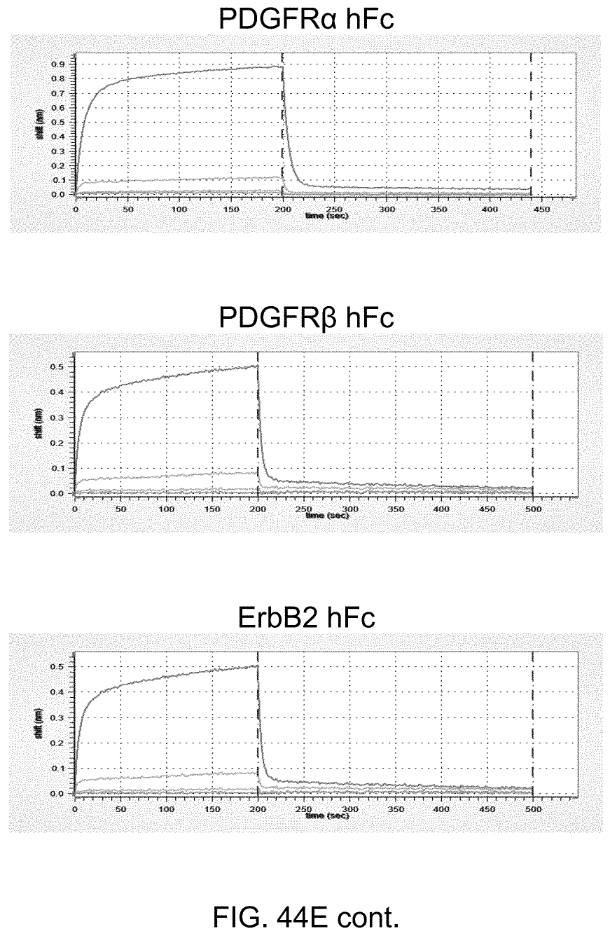

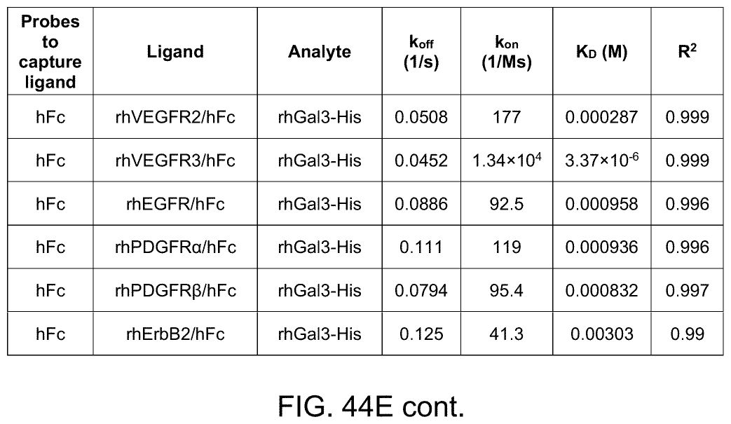

As depicted in FIG. 44A-D, no significant binding of the growth factor receptors was observed to plates without GAL3 coating. Similarly, the tumor cell surface receptors failed to bind uncoated ELISA wells. In contrast, VEGFR2, VEGFR3, PDGF R alpha (PDGFRa), PDGF R beta (PDGFRb), Her2 (ErbB2), FGFR1 alpha IIIb, FGFR1 alpha IIIc, FGFR3 IIIc, FGFR4, HGFR, TNF sRI, CTLA4, PDL1 and CD47 strongly bound GAL3-coated wells, whereas VEGFR1 and FGFR2 alpha IIIb did not show binding activity.

To further explore this observation, the kinetics of GAL3 assembly and disassembly with VEGFR2, VEGFR3, EGFR, PDGFRa, PDGFRb and ErbB2 were evaluated by surface plasmon resonance (FIG. 44E). Briefly, kinetics experiments were performed on Probe-Life Gator at 25° C. Anti-human Fc probes of tumor surface receptor proteins were used to load cell surface receptor/huFc fusion proteins (2.0 mg/mL) for 300 sec, then the loaded probes were dipped into a dilution series of human Galectin-3-His (Acro GA-H5129, starting from 1000 nM, 1:4 dilution for 4 points) for 200 sec, followed by dissociation for 240 sec. The binding affinities were characterized by fitting the kinetic sensorgrams to a monovalent binding model (1:1 binding).

### Example 26. Antibodies Targeted to the N-Terminal Domain or TRD of Gal3 Block Binding of Gal3 to Tumor Cell Surface Receptors

To identify GAL3-binding antibodies with the capacity to block the assembly of GAL3 and tumor cell surface receptors, purified GAL3 and tumor cell surface receptor proteins (EGFR, VEGFR2, VEGFR3, PDGFR alpha, and PDGFR beta) were incubated in the presence of a panel of GAL3-targeted monoclonal antibodies, with non-specific control antibodies, or without antibody, and protein interaction was evaluated by ELISA. Briefly, human galectin-3 protein (R&D Systems, 8259-GA) was diluted in PBS to a concentration of 3 μg/ml and added to the wells of a 96-well ELISA plate. After incubating the plate at 4° C. overnight, the plate was washed three times with PBST. The plate was then blocked for an hour with 2% BSA in PBST at room temperature with gentle rocking. Thereafter, the 2% BSA in PBST was discarded and 30 μl of control or anti-GAL3 antibodies at 20 μg/ml were added to each well, followed by the addition of 30 μl of 6 μg/ml of several recombinant human growth factor receptors, including recombinant human EGFR, VEGFR2, VEGFR3, PDGFR alpha, and PDGFR beta. The respective plates were incubated for an hour at room temperature with gentle rocking. Thereafter, the plate was washed three times with PBST. Peroxidase AffiniPure F(ab′)₂ Fragment Goat Anti-Human IgG (H+L) polyclonal antibody was then added to the wells at 1:4000 dilution in 2% BSA in PBST. The plate was incubated at room temperature for an hour with gentle rocking and then washed three times with PBST. TMB substrate was then added to each well. The reaction was stopped with 1M HCl and read using a plate reader (Molecular Devices, SpectraMax M4) at absorbance of 450 nm.

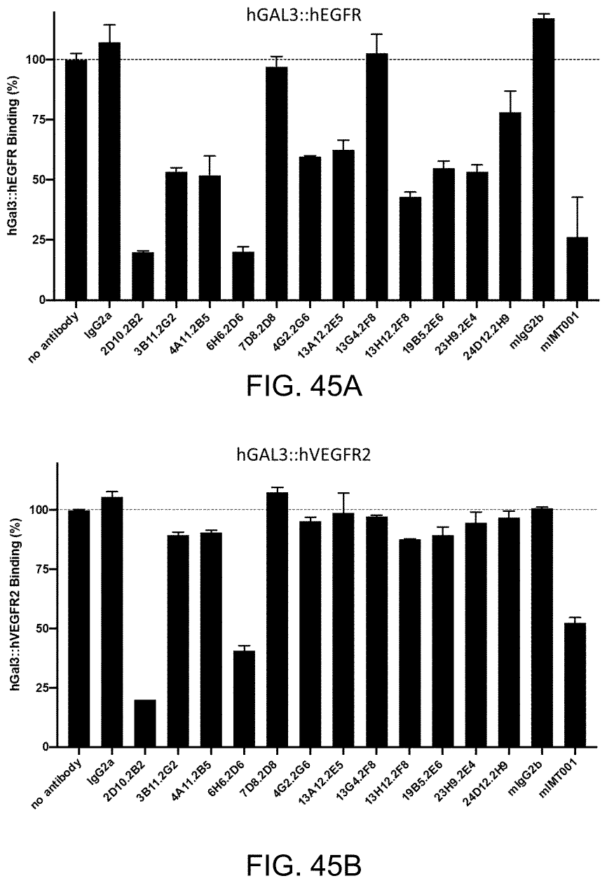

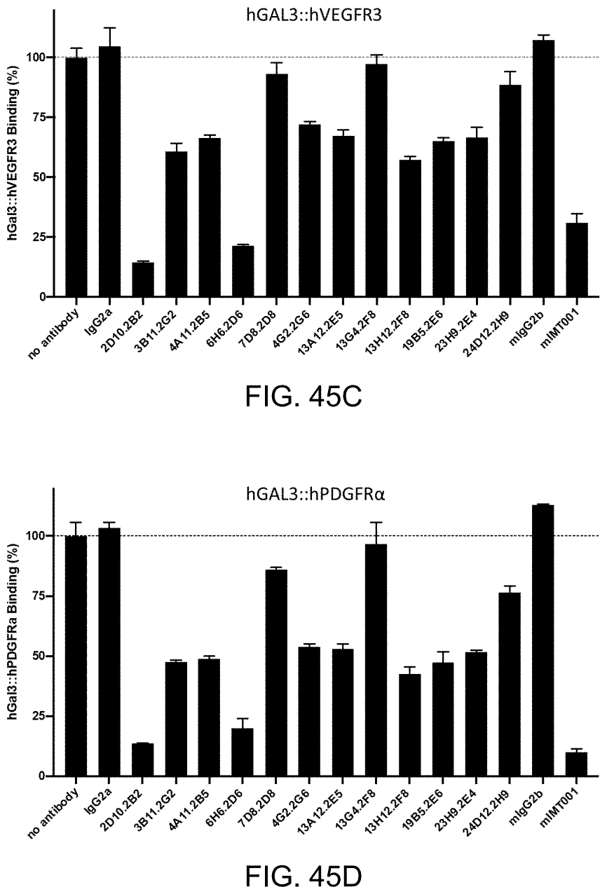

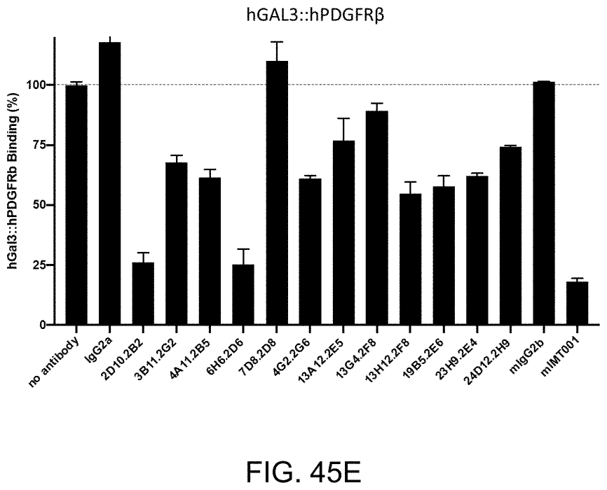

As depicted in FIG. 45A-E, GAL3-targeted antibodies exhibited a range of inhibitory activity against the binding of GAL3 to the growth factor receptors tested. One particular antibody, 2D10.2B2 displayed over 70% blocking of GAL3 assembly with all cell surface receptors tested. Three antibodies exhibited <30% inhibitory activity of the assembly of GAL3 with all tumor cell surface receptors tested, including 7D8.2D8, 24D12.2H9, 13G4.2F8. For the assembly of GAL3 with EGFR, three antibodies exhibited >70% blocking, including 6H6.2D6, 2D10.2B2, murine IMT001 (mIMT001). Several others displayed blocking between 30 and 70%, including 13A12.2E5, 3B11.2G2, 13H12.2F8, 23H9.2E4, 4A11.2B5, 19B5.2E6, 4G2.2G6. Three others displayed blocking <30%, including 7D8.2D8, 24D12.2H9, 13G4.2F8. For the assembly of GAL3 with VEGFR2, only 2D10.2B2 displayed blocking greater than 70%. Two antibodies displayed blocking between 30 and 70%, including mIMT001 and 6H6.2D6. Several displayed blocking less than 30%, including 7D8.2D8, 24D12.2H9, 13G4.2F8, 13A12.2E5, 3B11.2G2, 13H12.2F8, 23H9.2E4, 4A11.2B5, and 19B5.2E6. For the assembly of GAL3 with VEGFR3, 2D10.2B2 and 6H6.2D6 displayed blocking >70%. Several other antibodies displayed blocking between 30 and 70%, including 13A12.2E5, 3B11.2G2, 13H12.2F8, 23H9.2E4, 4A11.2B5, 19B5.2E6, and mIMT001. Several others displayed blocking <30%, including 7D8.2D8, 24D12.2H9, 13G4.2F8, and 4G2.2G6. For the assembly of GAL3 with PDGFR alpha, 6H6.2D6, 2D10.2B2, mIMT001 displayed blocking >70%. Several other antibodies displayed blocking between 30 and 70%, including 13A12.2E5, 3B11.2G2, 13H12.2F8, 23H9.2E4, 4A11.2B5, 19B5.2E6, and 4G2.2G6. Several others displayed blocking less than 30%, including 7D8.2D8, 24D12.2H9, and 13G4.2F8. For the assembly of GAL3 and PDGFR beta, 6H6.2D6, 2D10.2B2, and mIMT001 displayed blocking >70%. Several other antibodies displayed blocking between 30 and 70%, including 3B11.2G2, 13H12.2F8, 23H9.2E4, 4A11.2B5, 19B5.2E6, and 4G2.2G6. Several others displayed blocking <30%, including 7D8.2D8, 24D12.2H9, 13G4.2F8, and 13A12.2E5. Collectively, these observations reveal that distinct classes of GAL3-targeted antibodies exist with varying ability to interfere with the association of GAL3 with various growth factor receptor proteins.

### Example 27. Antibodies that Block Interaction of Gal3 with Tumor Cell Surface Receptors do not Bind to the C-Terminal Domain of Gal3

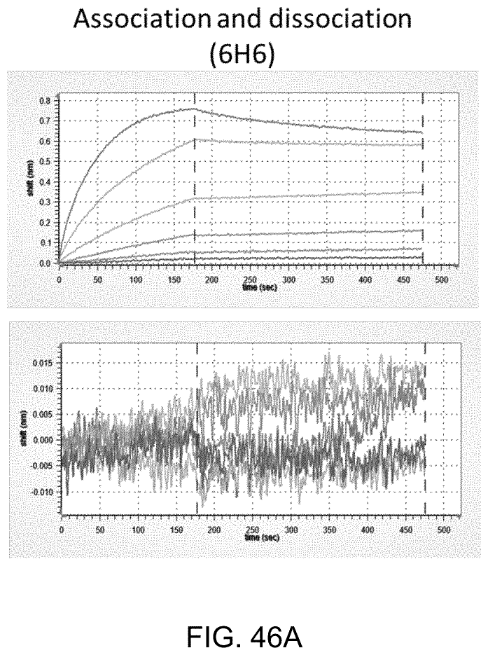

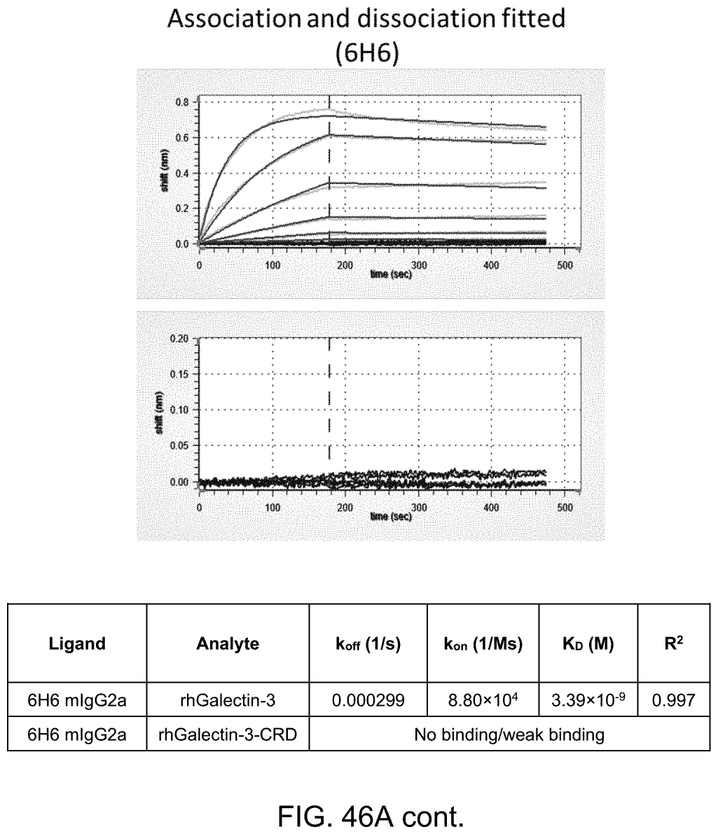

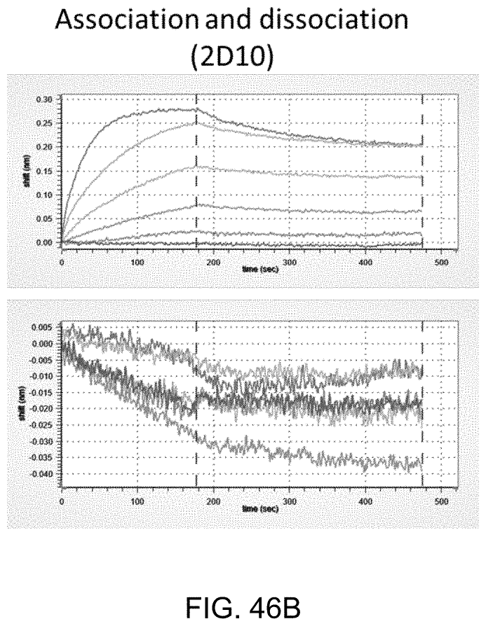

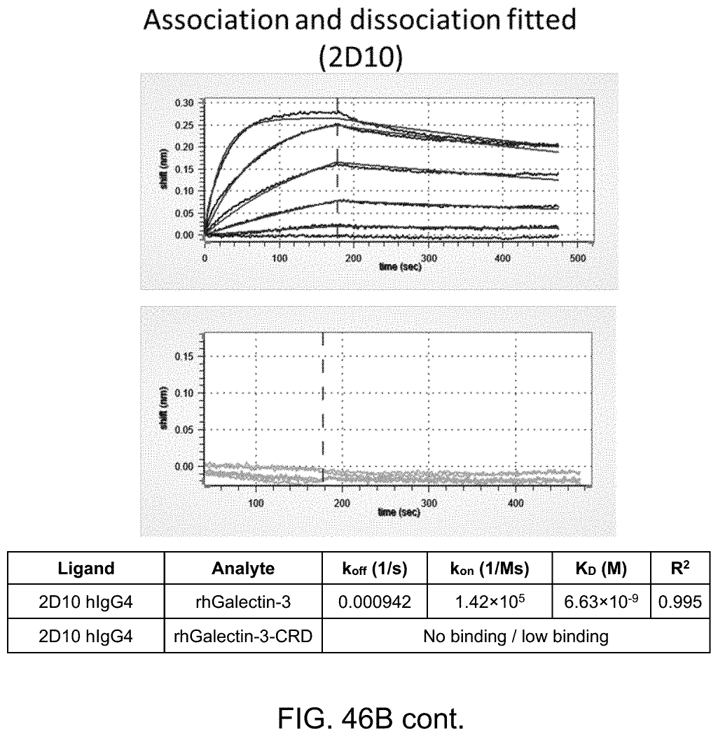

To determine whether GAL3-binding antibodies that block EGFR:GAL3 interaction do not bind to the C-terminal portion of GAL3, kinetics binding experiment was performed employing recombinant in house full-length human rhGalectin-3 (GAL3-His-tagged) or C-terminal domain of rhGal-3C (SinoBio #ME13SE2105 IM1-2, containing the C-terminal amino acids 112-250). hFc probes were used to load purified 6H6 mIgG2a (5 μg/mL) or 2D10 hIgG4 (5 μg/mL) onto a Gator biosensor (Probe Life, Palo Alto, Calif.) for 300 seconds (6H6) or 180 seconds (2D10) respectively, then the loaded probes were dipped into a dilution series of recombinant full-length human GAL3-His or rhGal-3C starting from 300 nM (for 6H6 clone) or 200 nM (for 2D10 clone) followed by dissociation for 300 sec. The binding affinities were characterized by fitting the kinetic sensorgrams to a monovalent binding model (1:1 binding). As depicted in FIG. 46A-B, both 6H6 and 2D10 anti-GAL3 clones exhibited binding to full length recombinant human galectin-3 with K_{D}=3.39×10⁻⁹ and K_{D}=6.63×10⁻⁹, respectively, but failed to bind the C-terminal region of galectin-3 (rhGAL3-C).

### Example 28. Antibodies Targeted to the N-Terminal Domain and TRD of Gal3 Inhibit Tumor Cell Growth In Vitro

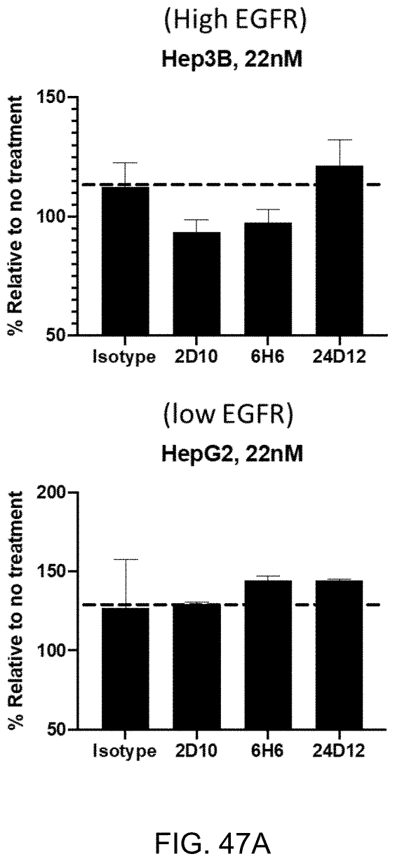

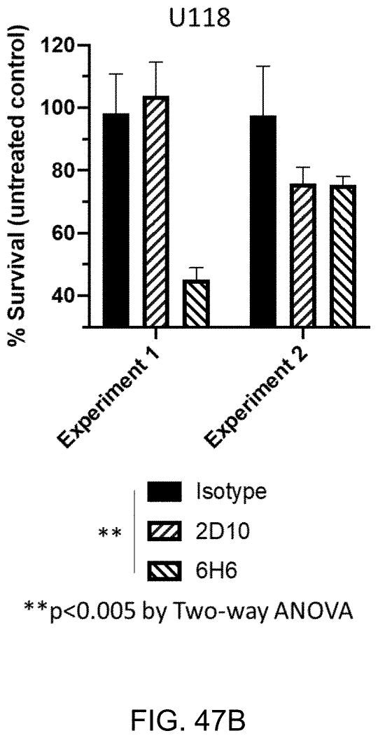

To assess activity of antibody in human hepatocellular carcinoma (HCC) cells, HepG2 (low expressors of EGFR) and Hep3B (high expressors of EGFR) cell lines were purchased from ATCC (Manassas, Va.) and maintained in cell culture media EMEM supplemented with 10% heat-inactivated FBS, penicillin and streptomycin. EGFR expression levels were validated in-house by flow cytorneLry (MACSQuant Analyzer 10) employing anti-EGFR antibody (AlexaFluor488 anti-human EGFR, Clone AY13: Biolegend, 352908, lot #B268316) and analyzed by FlowJo v10.6.2. HepG2 or Hep3B cells (5×10⁴ cells/well) were incubated with antibodies (two exemplary clones were used—2D10 and 6H6) in concentration ranging from OnM to 67 nM, and cultured a 96-well plate at 37° C. for at least 24 hours. Cell viability was assessed using an MTS (3-(4,5-dimethylthiazol-2-yl)-5-(3-carboxymethoxyphenyl)-2-(4-sulfophenyl)-2H-tetrazolium, inner salt) tetrazolium/formazan assay (Promega, Madison, Wis.). As depicted in FIG. 47A, EGFR-high Hep3B human HCC cell exhibited greater sensitivity compared to EGFR-low HepG2 cells to the treatment with anti-GAL3 antibodies that block galectin-3 interaction with tumor cell surface receptors (2D10 and 6H6 clone) apparent by reduced cell survival. In contrast, an anti-galectin-3 antibody that retains galectin-3 binding but does not block interaction of this lectin with tumor cell surface receptors (clone 24D12) failed to kill HCC tumor cells. Untreated wells were used as a reference control. Hep3B was analyzed in quadruplicates and HepG2 in duplicates.

To further assess activity of antibody in another model of human cancer—glioblastoma (GBM), the U118 cell line was purchased from ATCC and maintained in cell culture media DMEM supplemented with 10% heat-inactivated FBS, penicillin and streptomycin. U118 cells (1×10⁴ cells/well) were plated, and treated the following day with anti-Gal3 antibodies or isotype control at a concentration of 10 μg/ml. After 72 hours of incubation, the cell viability was assessed by CellTiterBlue viability assay (Promega, G8081) at absorbance of 560 nm by SpectraMax (Molecular Devices) following three-hour incubation with the reagent. Untreated wells were used as a reference control. As depicted in FIG. 47B, the human GBM cell line U118 exhibited sensitivity to anti-galectin-3 treatment (here, clone 6H6) resulting in reduced cell number when compared to control isotype treated group. Two independent experiments were conducted, each experiment performed in quadruplicates.

### Example 29. Antibodies Targeted to the N-Terminal Domain or TRD of Gal3 Inhibit Brain Tumor Growth in Combination with Standard of Care Treatments In Vitro

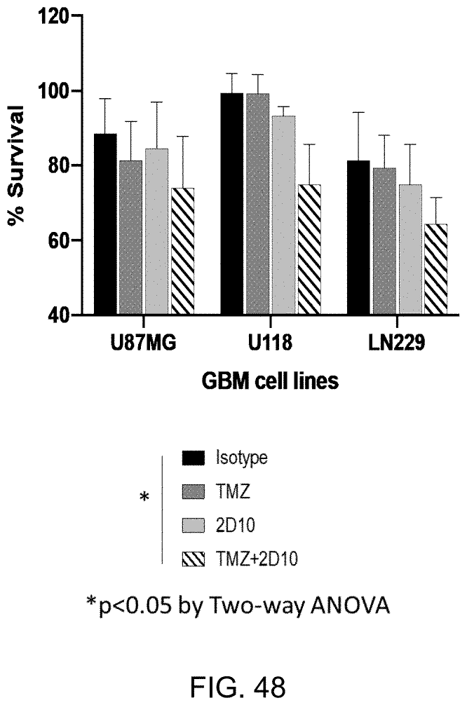

To assess activity of antibody in human GBM cells in combination with the standard of care in vitro, U87MG, LN229 and U118 cell line were purchased from ATCC and maintained in cell culture media DMEM supplemented with 10% heat-inactivated FBS, penicillin and streptomycin. Human GBM cells (1×10⁴ cells/well) were plated, and treated the following day with antibodies in concentration 10 μg/ml, alone or in combination with temozolomide (TMZ) (Sigma-Aldrich, 76899) in the concentration of 100 μM. TMZ was reconstituted in DMSO. After 72 hours of incubation, the cell viability was assessed by CellTiterBlue viability assay (Promega, G8081) at absorbance of 560 nm by SpectraMax (Molecular Devices) following three-hour incubation with the reagent according to manufacturer's protocol. Untreated wells were used as a reference control. As depicted in FIG. 48, combinatorial treatment of tumor cells by anti-GAL3 antibody (here, clone 2D10) with temozolomide resulted in reduction of glioma cell survival when compared to isotype treated control. Human GBM cell lines exhibited increased sensitivity to anti-galectin-3 treatment in combination with TMZ when compared to the wells treated with control IgG (*p<0.05 by two way ANOVA, or *p=0.0247 by paired t-test). Results were expressed as mean±SEM. The experiment was conducted in quadruplicates.

### Example 30. Antibodies Targeted to the N-Terminal Domain or TRD of Gal3 Retard Brain Tumor Growth in Combination with Standard of Care Treatments In Vivo

To validate anti-tumor efficacy of anti-GAL3 antibodies in combination with the standard of care for GBM patients, temozolomide, Albino C57B16 mice (The Jackson laboratory) were orthotopically transplanted with murine glioma cells GL261 that stably express firefly luciferase gene (GL261-LUC).

To generate GL261-LUC cells, GL261 murine glioblastoma cell line (DSMZ, no. ACC 802) was transduced with pLL-CMV-Luciferase-T2A-Puro Lenti-Labeler™ Pre-packaged Virus (SBI, catalog No. LL150VA-1) employing transduction reagent (TransDux MAX Transduction reagent, SBI, catalog No. LV860A-1) according to the manufacturer's instructions. The transduced cells were maintained in cell culture medium (90% DMEM, 10% FBS, 4 mM glutamine) containing 5 μg/mL of Puromycin for the duration of two weeks.

GL261-LUC (3×10⁵) were transplanted into the forebrain of the recipient animals (1 mm posterior of bregma, 2 mm lateral of midline and 3.5 mm deep, employing a stereotaxic frame (Stoelting, 51725D). Three days after the transplantation, the animals were subjected to the following treatment: Group1: Isotype control (mIgG2a), Group2: Temozolomide, Group3: anti-Gal3 antibody clone 2D10, Group4: anti-Gal3 antibody clone 2D10 and Temozolomide. The animals were dosed three times with 2D10 (mIgG2a isotype) or control mIgG2a antibodies at concentration 10 mg/kg at Q3D regime of treatment. For the temozolomide treatment group, the animals were subjected to 5 mg/kg treatment of TMZ or vehicle control (10% DMSO in PBS) by oral gavage for five consecutive days.

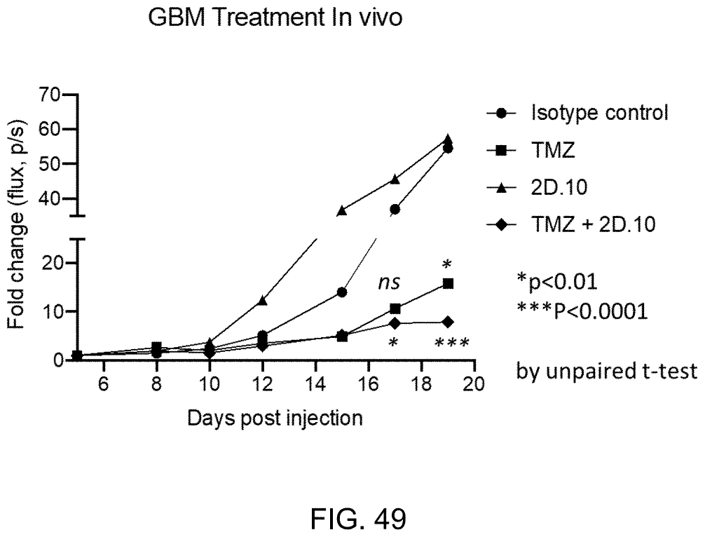

The tumor progression was monitored by imaging of tumor luminescence. Luminescent images were obtained 10 min after injection with XenoLight D-Luciferin-K+Salt Bioluminescent Substrate (Perkin Elmer, cat #122799, prepared according to manufactured protocol) using an IVIS-Spectrum CT Imaging System (PerkinElmer). Bioluminescence was measured as photons/second and reported as the fold difference over time, where the first measurement at treatment start was set to 1. Results were expressed as mean±SEM. The statistical analysis was performed in comparison with an IgG control group. As depicted in FIG. 49, animals subjected to the anti-Gal3 antibody (2D.10, p<0.01) or the combinatorial treatment (TMZ+2D.10, p<0.0001) demonstrated significant reduction in tumor burden as monitored by bioluminescence compared to control mice at day 17 post tumor-cell transplantation when compared to isotype control treated group.

### Example 31: Discovery of Antibodies with GAL3 Binding Activity

GAL3-binding antibodies were identified with the following protocol. Balb/C, FVB, and CD-1F mice were inoculated at 7 day intervals with 50 ug of GAL3 protein fused to a linker-spaced 6-histidine tag, GAL3-ECD-His, (Acro GA3-H5129; Lot #819-43PS1-5E.) in combination with a TLR agonist adjuvant mix (50 μg MPL, 20 μg CpG, 10 μg Poly(I:C) and 10 μg R848) for 3 repetitions, followed by an inoculation with 50 ug of GAL3-His alone administered subcutaneously to the inguinal, back of the neck and base of the tail sites as well as hock and intraperitoneal sites. Animals were sacrificed in accordance with IACUC protocol and spleen, femurs, and lymph nodes (axillary, accessory axillary, mediastinal, superficial inguinal, iliac, sacral and popliteal) were harvested. A single cell suspension of immunized lymph node (LN), spleen and bone marrow cells were obtained using 2 sterile frosted glass slides in a tissue culture petri dish with 15 mL DMEM. Bone marrow was extracted from femurs via end-cap flushing with a 5 mL syringe fitted with an 18-gauge needle. Cells from 3 animals were pelleted with 5 minutes of centrifugation at 1200 RPM, resuspended in 10 mL of DMEM (GIBCO 10564-011) and nucleated cells were enumerated by hemocytometer count. Cells were pelleted at 1200 RPM and were resuspended in SC-Buffer (PBS, 2% FBS and 1 mM EDTA), and plasma cells were isolated with an EasySep™ Mouse CD138 Positive Selection Kit (StemCell Technologies) with the manufacturer recommended protocol. Enriched CD138-positive cells were pelleted with 5 minutes of centrifugation at 1200 RPM, resuspended in 50 mL electrofusion buffer (Eppendorf 940-00-220-6) and were enumerated. Separately, SP2/0-mIL6 myeloma cells (ATCC CRL2016) were pelleted with 5 minutes of centrifugation at 1200 RPM, resuspended in 50 mL electrofusion buffer and were enumerated. Myeloma cells and CD138-positive plasma cells were combined at a 1:1 ratio, volume was expanded to 50 mL with electrofusion buffer, cells were pelleted with 5 minutes of centrifugation at 1200 RPM and supernatant was discarded. After a repeated step of washing and pelleting in electrofusion buffer, cells were resuspended in electrofusion buffer to a concentration of 10×10\hat{ }6 cells/ml, up to 9 mL of cell suspension was added to a BTX electrofusion chamber, and cells were fused with an 800V electrofusion protocol. Fused cells were rested for 5 minutes, transferred to a tissue culture dish containing 40 mL medium MM (DMEM, 15% FBS, 1% glutamax and 1% Pen/Strep), incubated for 1 hour at 37C, 8% CO2, resuspended with a pipette, pelleted with 5 minutes of centrifugation at 1200 RPM, resuspended in ClonaCell HY Liquid HAT Selection Medium (StemCell Technologies), and plated in 96-well tissue culture flat bottomed plates. After 10 days, supernatants were sampled and evaluated for binding to isolated GAL3 by ELISA. 50 ul of 0.1 μg/mL GAL3-ECD-His, (Acro GA3-H5129; Lot #819-43PS1-5E) resuspended in diluent (PBS with 0.5% BSA) was added to each well for 45 minutes, supernatant was discarded and plates were washed with phosphate buffered saline (PBS) with 0.05% Tween20. 50 ul of 1:5 dilution of hybridoma supernatant in diluent was added to each well for 1 hour, followed by 5 successive 300 ul washes with PBS/0.05% Tween20, after which a 1:3000 dilution of goat anti-mouse Fc-specific antibody conjugated to horseradish peroxidase (Novex A16090) in 50 ul of diluent was added to each well for 1 hour followed by 5 successive 300 ul washes with PBS/0.05% Tween20. Following washing, 50 ul of ABTS (Novex #00-202-4) was added to each well for 20-30 minutes, prior to readout on a spectrophotometer (Molecular Devices) at absorbance of 405 nm.

### Example 32. Gal3-Targeted Antibodies Bind to Distinct Epitopes of Gal3

To identify the epitopes to which Gal3 antibodies bound, a library of 20 amino acid peptides representing portions of Gal3, summarized in FIG. 17, was produced and the ability to bind Gal3 antibodies was evaluated by ELISA.

At least 2 μg/ml of hGal3 peptide in 50 μl of PBS or 0.1 μg/ml of full-length human Gal3 protein (GenScript) and human Galectin-3 protein (Acro Biosystems, GA3-H5129) were diluted in PBS (Corning, 21-030-CM) to concentrations of at least 2 μg/ml or 0.1 μg/ml, respectively, and added to the wells of a 96-well ELISA plate (Thermo Fisher, 44-2404-21). After incubating the plate at 4° C. overnight, the plate was washed three times with PBST (PBS with 0.05% Tween 20 [VWR, 0777]). The plate was then blocked for an hour with 2% BSA (EMD Millipore, 126609) in PBST at room temperature with gentle rocking. Thereafter, the 2% BSA in PBST was discarded and human Galectin-3 hybridoma supernatants or antibodies were diluted in 2% BSA in PBST to concentrations of at least 0.1 μg/ml and added to the wells. The plate was incubated for an hour at room temperature with gentle rocking and then washed three times with PBST. Afterwards, Goat Anti-Mouse IgG-HRP (Jackson ImmunoResearch, 115-036-1461) or Goat Anti-Rat IgG HRP (abcam, ab205720) diluted in 2% BSA in PBST (1:4000) were added to the wells. The plate was incubated for 30 minutes to 1 hour at room temperature with gentle rocking and then washed three times with PBST. TMB substrate (Thermo Scientific, 34029) was then added to each well. The reaction was stopped with 1M HCl (JT Baker, 5620-02) and read using a plate reader (Molecular Devices) at absorbance of 450 nm.

Binding of Gal3-binding antibodies to the peptide array was observed at multiple locations, with the majority of binding observed in peptides 1-8, summarized in FIG. 17. Six separate Gal3-binding antibodies (6H6.2D6, 20H5.A3, 20D11.2C6, 19B5.2E6, 15G7.2A7, 23H9.2E4) all bound peptide 1 of Gal3, corresponding to amino acids 1-20 of Gal3, ADNFSLHDALSGSGNPNPQG (SEQ ID NO: 3). Similarly, three separate Gal3-binding antibodies (4G2.2G6, 3B11.2G2, and 13A12.2E5) bound peptide 4 of Gal3, corresponding to amino acids 31-50 of Gal3, GAGGYPGASYPGAYPGQAPP (SEQ ID NO: 6). Further, thirteen Gal3-binding antibodies (IMT001, 846T.1H2, 13H12.2F8, 19D9.2E5, 14H10.2C9, 2D10.2B2, 4A11.2B5, 846.2H3, 846.1F5, 3B11.2D2, and 13A12.2E5) all bound peptide 6 of Gal3, corresponding to amino acids 51-70 of Gal3, GAYPGQAPPGAYPGAPGAYP (SEQ ID NO: 8). Additionally, eleven Gal3-binding antibodies (6H6.2D6, 20H5.A3, 20D11.2C6, 13H12.2F8, 19B5.2E6, 23H9.2E4, 15G7.2A7, 19D9.2E5, 14H10.2C9, 7D8.2D8, 15F10.2D6 and 846.14A2) all bound peptide 7 of Gal3, corresponding to amino acids 61-80 of Gal3, AYPGAPGAYPGAPAPGVYPG (SEQ ID NO: 9).

As illustrated in FIG. 17, peptides 4, 5, 6, and 7 share repeated amino acid sequences comprised of proline-glycine (PG) and tyrosine-proline-glycine (YPG), indicating a common feature that may explain the ability of Gal3-targeted antibodies to bind to multiple Gal3 peptides. Further, the amino acid sequence glycine-x-tyrosine-proline-glycine (GxYPG), where x may be the amino acids alanine (A), glycine (G), or valine (V), is shared in peptides 4, 6, and 7, each of which possess two such sequences separated by 3 amino acids. Accordingly, the presence of two GxYPG sequences in close apposition is likely predictive of the ability to bind Gal3-targeted antibodies. Additionally, the Grantham distance of alanine, glycine, and valine is Ala-Val: 64, Ala-Gly: 60, Val-Gly: 109, thereby predicting that amino acids with similarly low Grantham distances may similarly be able to substitute at the variable region, including proline and threonine.

### Example 33. Anti-Gal3 Antibodies for Use as a Supplement for Standard of Care Treatment of Cancer

A patient is selected who has a cancer. The cancer may be a brain cancer, breast cancer, colorectal cancer, kidney cancer, liver cancer, lung cancer, pancreatic cancer, bladder cancer, stomach cancer, or a hematological malignancy, or another cancer not listed here. In some embodiments, the anti-Gal3 antibody or binding fragment thereof is selected from the group consisting of: 6H6.2D6, 20H5.A3, 20D11.2C6, 4G2.2G6, 13H12.2F8, 19B5.2E6, 15G7.2A7, 23H9.2E4, 19D9.2E5, 2D10.2B2, 4A11.2B5, 14H10.2C9, 3B11.2G2, 13A12.2E5, 7D8.2D8, 15F10.2D6, 23B10.2B12, 12G5.D7, 24D12.2H9, 6B3.2D3, 13G4.2F8, 9H2.2H10, 846.1F5, 846.2H3, 846T.1H2, TB001 (IMT001), TB006 (4A11.H3L1), 4A11.H1L1, and 4A11.H4L2, or binding fragment thereof. The anti-Gal3 antibody or binding fragment thereof is administered enterally, parenterally, intravenously, intramuscularly, intra-arterially, intradermally, subcutaneously, intraperitoneally, intraventricularly, intrathecally, or intracranially. In some embodiments, the anti-Gal3 antibody or binding fragment thereof is administered as at least one dose at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. The doses are administered every 1, 2, 3, 4, 5, 6, 7, 8, 9, 10, 11, 12, 24, 36, or 48 days or weeks or any time within a range defined by any two of the aforementioned times.

The patient experiences a reduction in cancer burden (e.g. tumor size and number) or an amelioration of symptoms such as symptoms of pain, discomfort, or inflammation following administration of the anti-Gal3 antibody or binding fragment thereof.

In some embodiments, the anti-Gal3 antibody or binding fragment thereof is administered as a supplement to a standard of care therapy for a cancer. In some embodiments, the standard of care treatment may include but is not limited to surgery, radiation, chemotherapy, targeted therapy, or immunotherapy. For example, for glioblastoma or astrocytoma, temozolomide is a standard of care chemotherapy. In some embodiments, the combination of the standard of care therapy and the anti-Gal3 antibody or binding fragment thereof has a greater positive effect in reducing cancer burden or ameliorating symptoms compared to the standard of care therapy alone. In some embodiments, the anti-Gal3 antibody or binding fragment affects symptoms (e.g. inflammation or other immune-related symptoms) different from what the standard of care therapy affects.

### Example 34. Sequences of Anti-Gal3 Antibodies

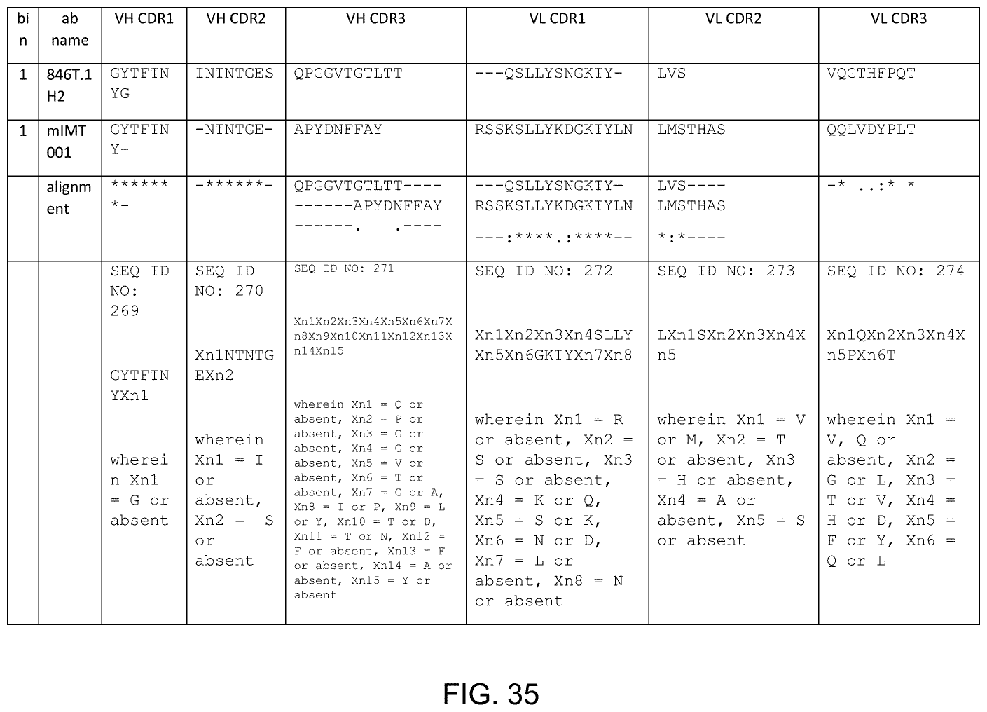

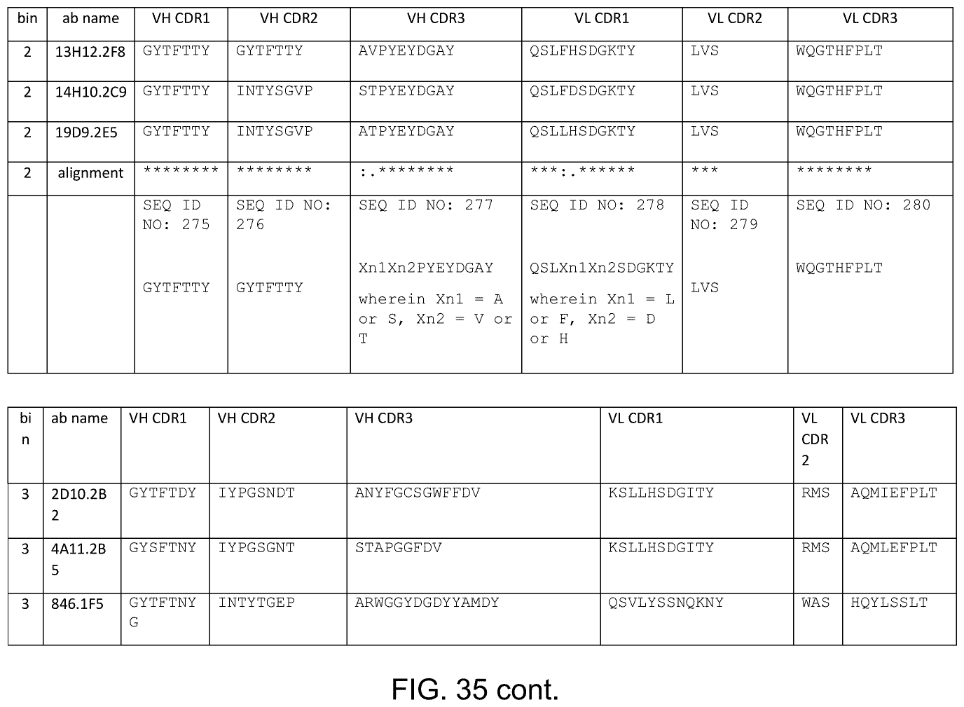

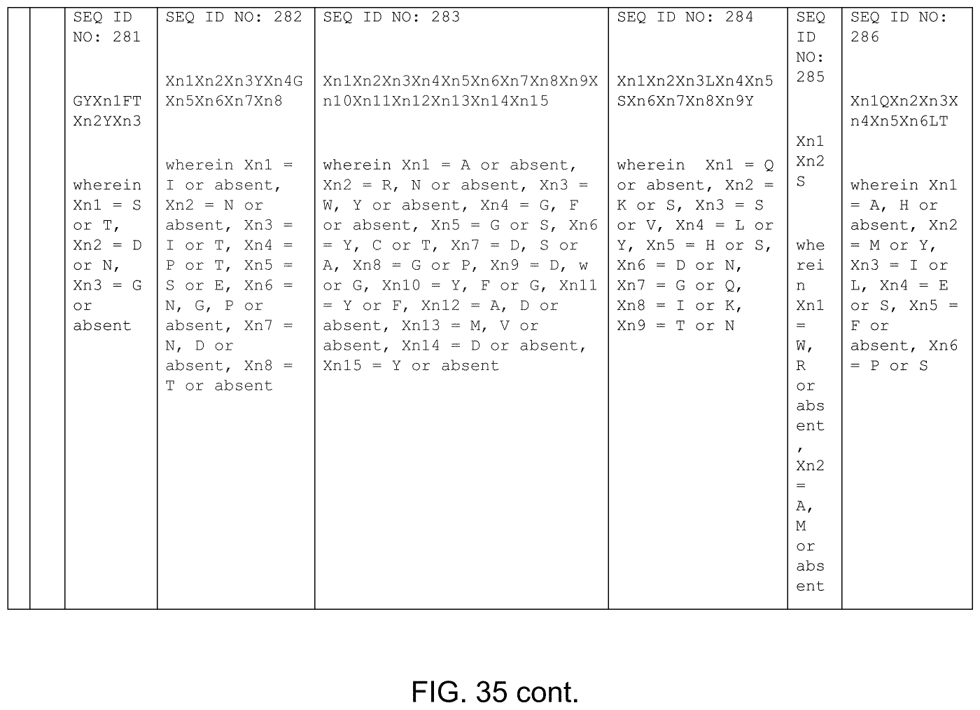

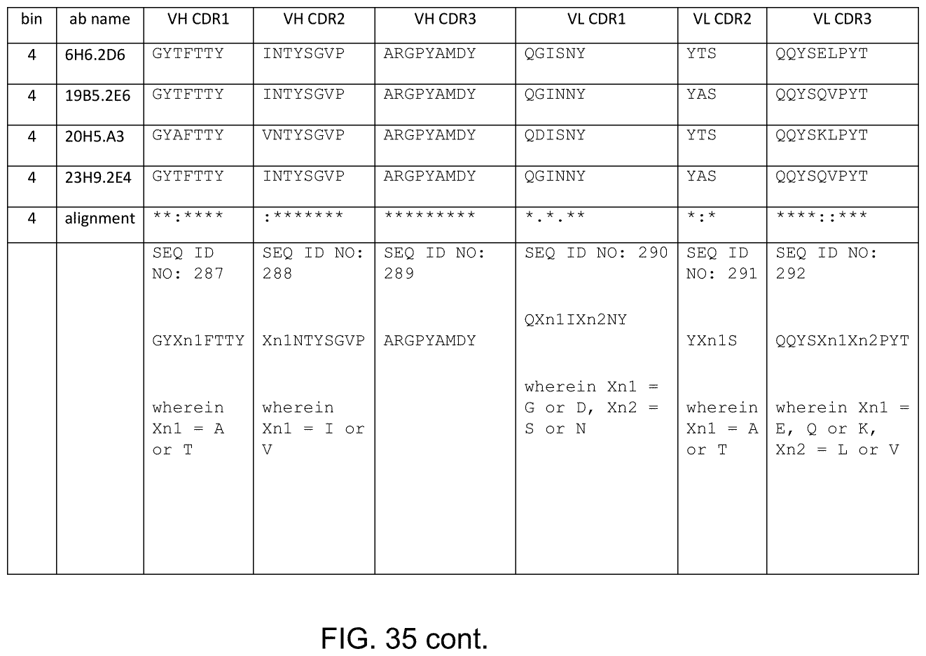

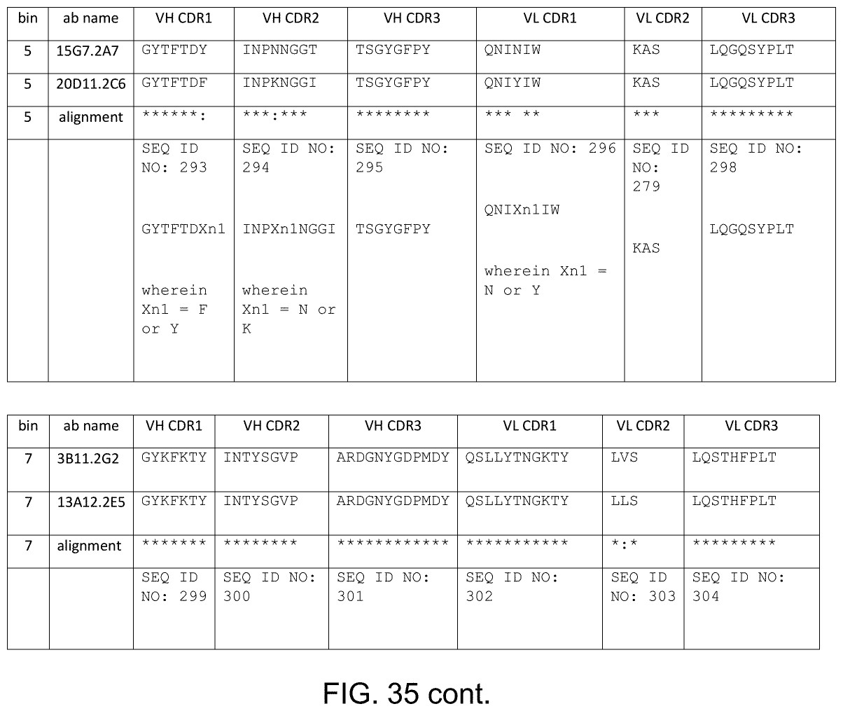

Complementarity determining regions of GAL3 binding antibodies from various bins were aligned using Clustal Omega (FIG. 35). Bin 1 antibodies shared significant homology in VH CDR1 and CDR2, as well as in regions of VL CDR1 and CDR3. Bin 2 antibodies shared significant homology in all CDRs examined, with relatively conservative A/S, V/T, H/D, and L/F substitutions observed. Bin 3 antibodies were somewhat more diverse, with significant sequence homology in CDR1, but relatively divergent in other CDR regions. Bin 4 antibodies shared significant homology in all CDRs examined, with relatively conservative A/T, I/V, D/G, S/N, QK, and V/L substitutions observed. Bin 5 antibodies also shared significant homology in all CDRs, with relatively conservative Y/F, N/K, substitutions observed in addition to less conservative T/I, N/Y substitutions. Finally, bin 7 antibody CDRs were observed to be nearly identical, with a single V/L substitution in VL CDR2 distinguishing 3B11.2G2 from 13A12.2E5. Alignments with any of the other sequences provided in FIG. 18-27 can be done with techniques known in the art.

### Example 35. Humanized Anti-Gal3 Antibodies have High Affinity for Gal3 of Different Species

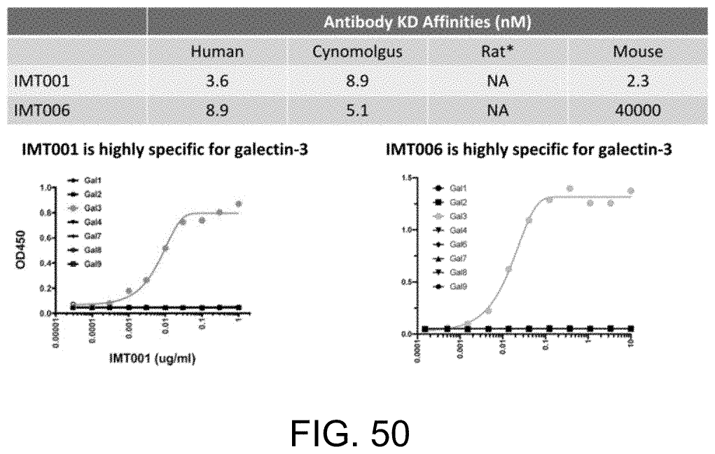

Humanized TB001 (IMT001) and TB006 (4A11.H3L1, IMT006-5), which were derived from mouse mAbs, both have high affinity for human (IMT001: 3.6 nM, 4A11.H3L1: 8.9 nM) and cynomolgus (IMT001: 8.9 nM, 4A11.H3L1: 5.1 nM) Gal3 (FIG. 50). IMT001 also has high affinity for mouse Gal3 (IMT001: 2.3 nM, 4A11.H3L1: 40000 nM).

### Example 36. TGF-β-Induced Pro-Collagen Production is Inhibited by Gal3 Blockage

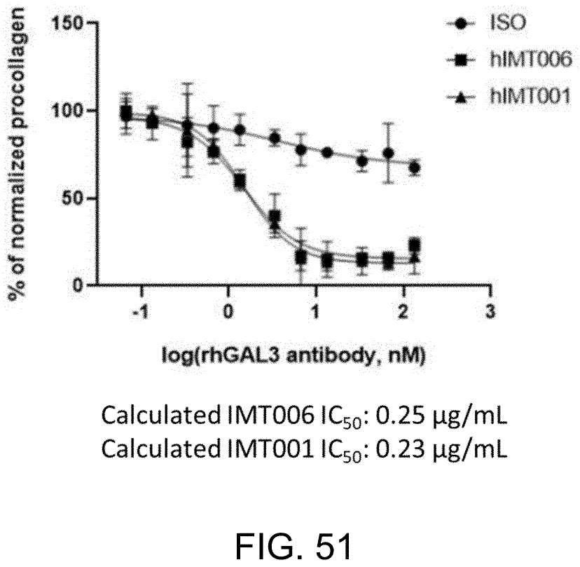

LX-2 hepatic stellate cells (Millipore, Cat #SCC064) were stimulated with 10 ng/mL TGF-β for 24 hours and treated with increasing concentrations of hIgG4 isotype, IMT001, or 4A11.H3L1 (IMT006-5). Pro-collagen expression was measured by Human procollagen kit (Abeam, Cat #ab229389). A significant reduction in pro-collagen production was observed in cells treated with either IMT001 or 4A11.H3L1 compared to isotype control, with an approximate maximum inhibition at 1 nM of antibody (FIG. 51). IC₅₀ of IMT001 was calculated to be 0.25 μg/mL and 4A11.H3L1 was calculated to be 0.23 μg/mL.

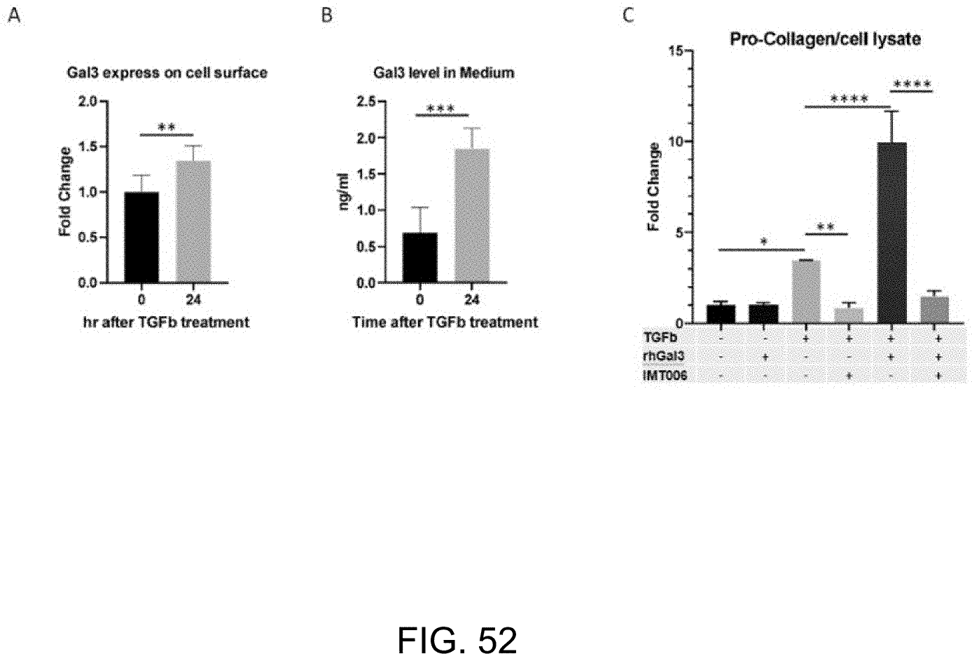

LX-2 cells stimulated with TGF-β exhibited an increase in Gal3 expressed on the cell surface and in the culture medium as a soluble form (FIG. 52). The anti-Gal3 antibody 4A11.H3L1 reduced pro-collagen protein formation when added to the cells after TGF-β stimulation alone or TGF-β stimulation in combination with exogenous Gal3.

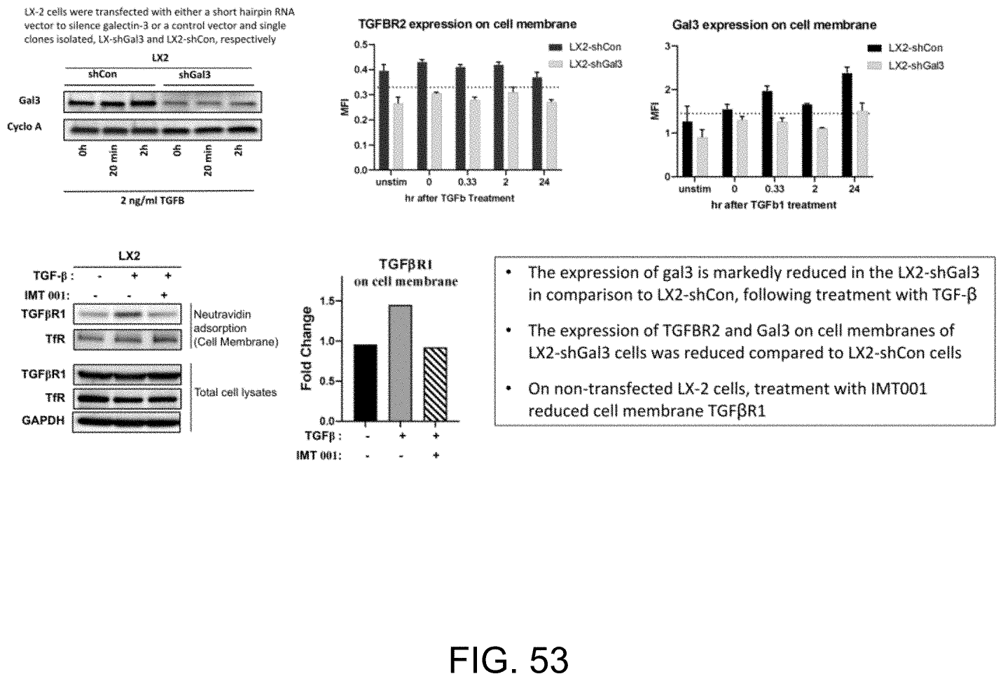

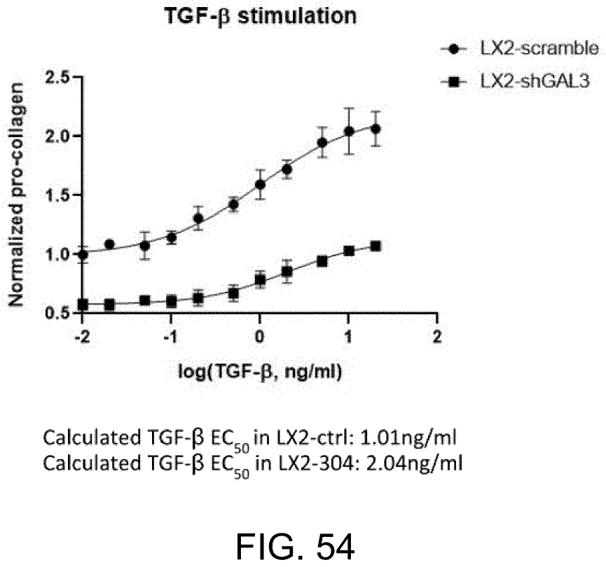

LX-2 cells were transfected with a Gal3 (LX2-shGal3) or control (LX2-shCon) short hairpin RNA (shRNA) construct. Expression of Gal3 was markedly reduced in LX2-shGal3 cells compared to LX2-shCon independently from TGF-β treatment. The expression of both TGFBR2 and Gal3 on the cell surface of LX2-shGal3 cells was reduced compared to LX2-shCon cells. The levels of TGFBR1 in non-transfected cells upon TGF-β treatment were reduced by IMT001 (FIG. 53). Knockdown of Gal3 with the shRNA (LX2-304) construct also reduced TGF-β induced pro-collagen secretion (FIG. 54). The calculated TGF-b EC₅₀ for pro-collagen production in LX2-304 (Gal3 knockdown cells) was 2.04 ng/mL whereas the TGF-b EC₅₀ for pro-collagen production in LX2-scramble (control cells) was 1.01 ng/mL.

### Example 37. Selected Anti-Gal3 Antibodies Exhibit Favorable Pharmacokinetics

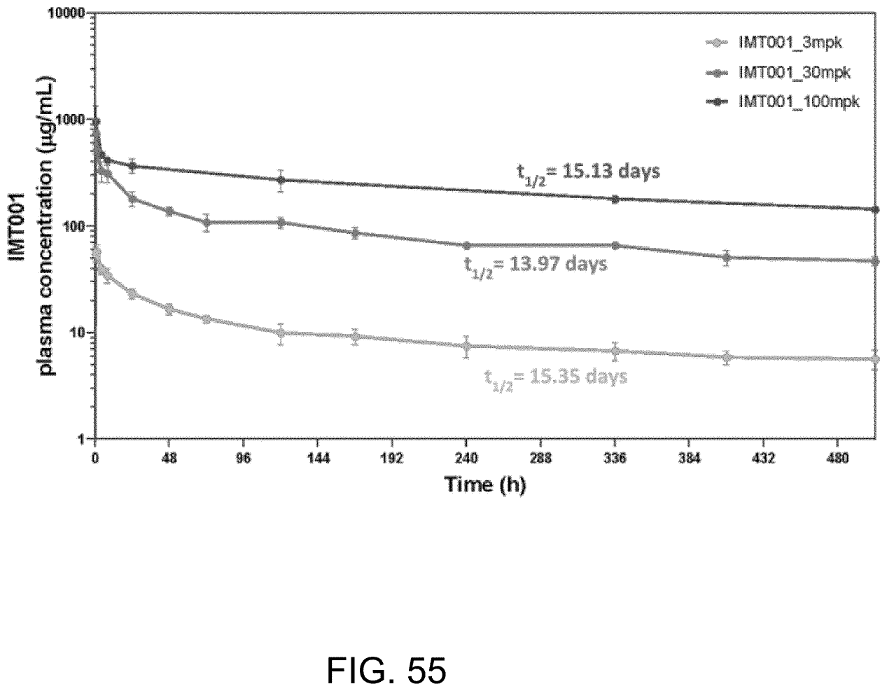

Rats were administered the anti-Gal3 antibody IMT001. The pharmacokinetics of IMT001 is dose proportional in rats with a half-life of approximately 2 weeks (FIG. 55).

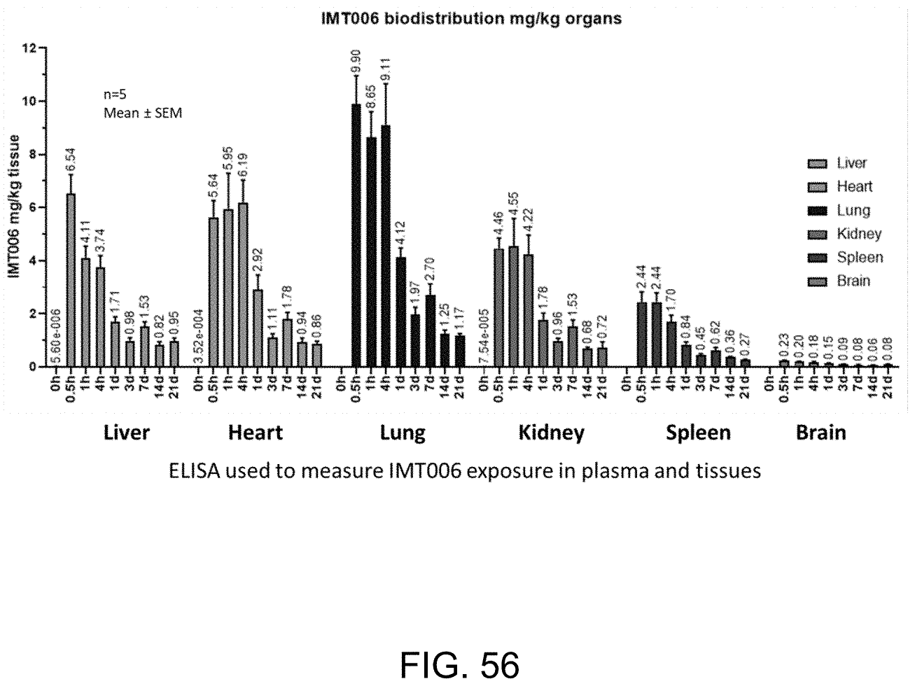

Mice were administered the anti-Gal3 antibody 4A11.H3L1 in a single injected dose. After time points at 0, 0.5, 1, 4, hours and 1, 3, 7, 14, 21 days, mice were euthanized, and plasma and organ tissue samples were taken. Biodistribution of 4A11.H3L1 was measured by ELISA, and organs that have a high incidence of fibrosis, such as the liver, kidney, and lung, have relatively high distribution of the antibody (FIG. 56). These results suggest that use of this antibody will be effective in targeting fibrotic diseases such as liver fibrosis, kidney fibrosis, and lung fibrosis.

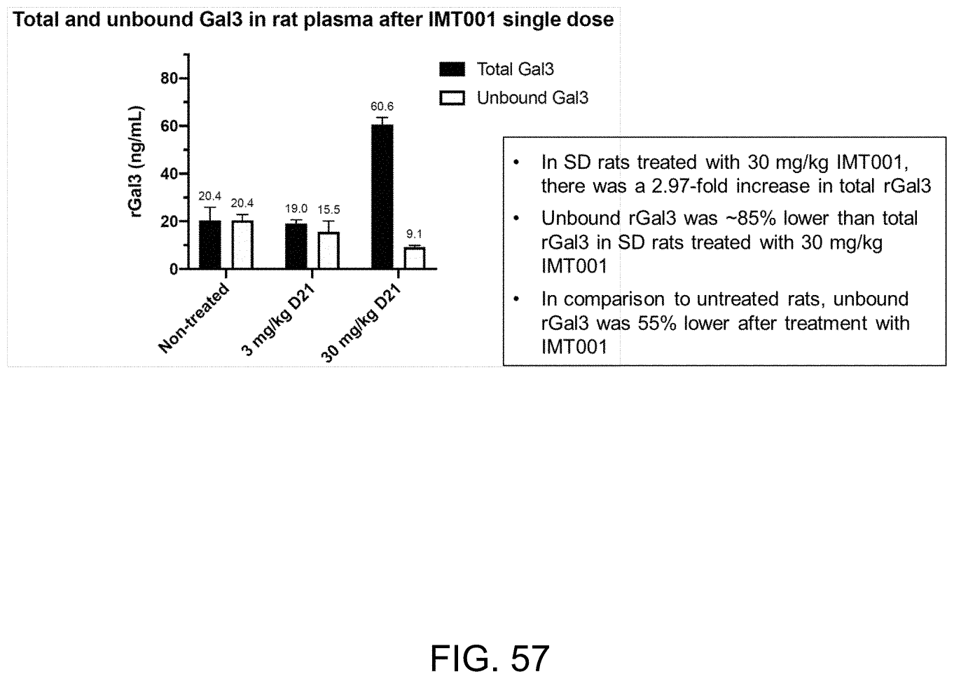

Rats were administered the anti-Gal3 antibody IMT001 at 10 mg/kg each for 10 total doses given intravenously. The first two doses were weekly, and then 3-10 were biweekly. Plasma samples were taken 3 days after the last dose. ELISA was used to measure total and unbound (soluble) Gal3 in the samples (FIG. 57). Total and unbound Gal3 plasma levels were nearly identical. In spontaneously hypertensive (SHR) rats treated with IMT001, a 2.97-fold increase in total Gal3 was observed and unbound Gal3 was ˜85% lower than total Gal3. In comparison to untreated rats, rats treated with 30 mg/kg IMT001 exhibited approximately 56% lower unbound Gal3. This shows that there is a dose-dependent reduction of unbound Gal3 following anti-Gal3 antibody administration.

### Example 38. Transcriptomics in MCD Mice Treated with Anti-Gal3 Antibody

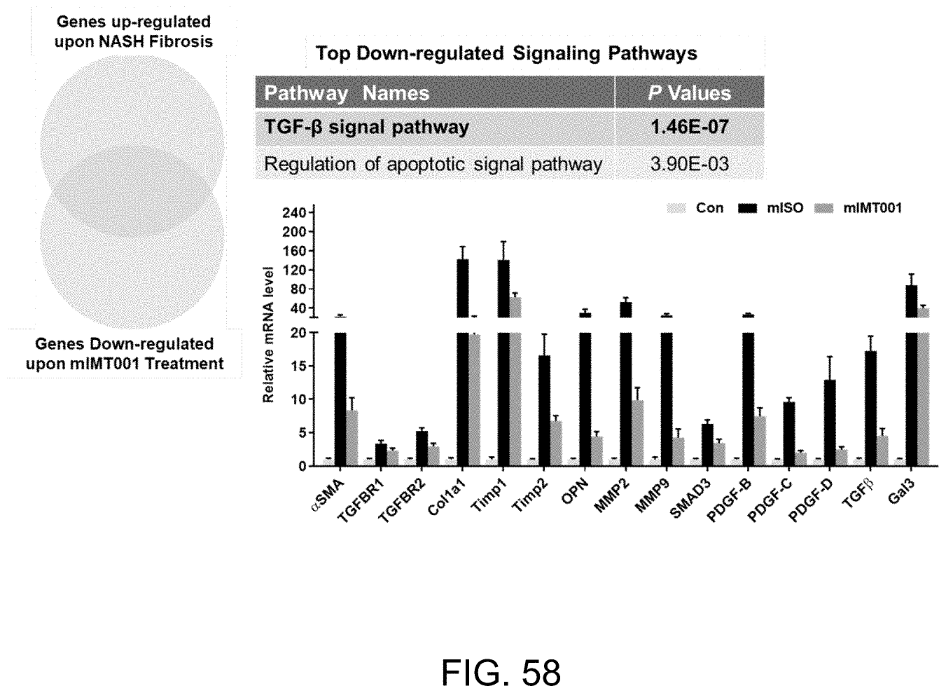

Methionine-choline deficient (MCD) mice were treated with murine IMT001 or control isotype. Liver samples were taken from the mice, and RNA-seq was performed to determine genes that are differentially expressed following murine IMT001 treatment (FIG. 58). These resultant genes were compared to genes up-regulated during NASH fibrosis. Expression of 225 genes was reduced by murine IMT001 treatment. Of these, genes involved in TGF-0 signaling were largely identified. This suggests that Gal3 modulates TGF-p-mediated pathways to reduce the effects of fibrosis.

### Example 39. Gal3 and TGFBR1 Expression is Co-Localized and Reduced Following Anti-Gal3 Antibody Administration

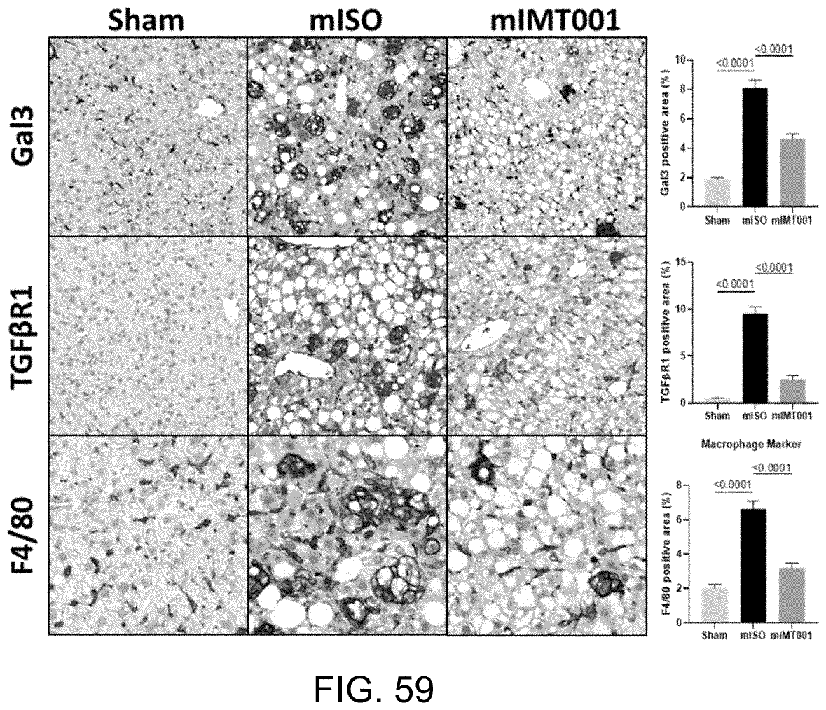

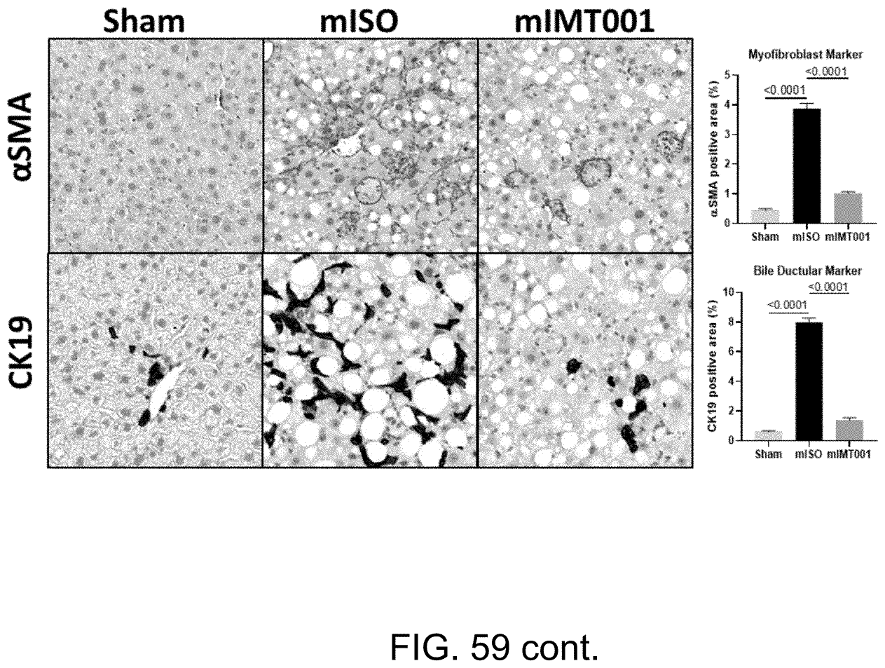

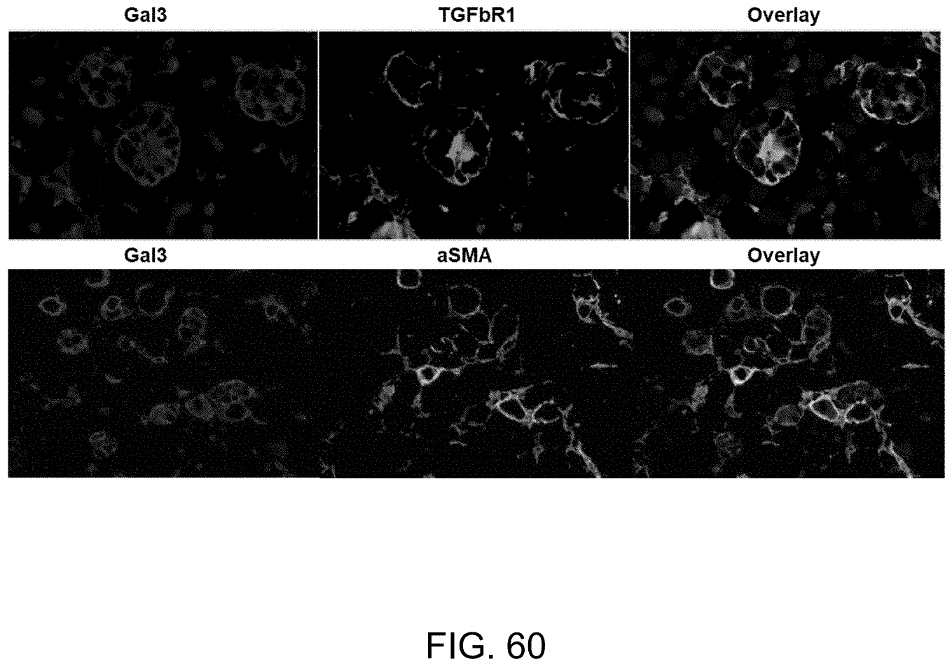

The expression of Gal3, TGFBR1, and α-SMA in MCD mice analyzed by immunofluorescence show colocalization of Gal3 with TGFBR1 and α-SMA suggesting Gal3 interaction by these two proteins (FIG. 60). MCD mice were treated with murine IMT001 or control isotype, and liver samples were analyzed by histochemistry (FIG. 59) to detect Gal3 and TGFBR1 levels. Mice treated with murine IMT001 exhibited a reduction in expression of Gal3, TGFBR1, F4/80 (macrophages), α-SMA (myofibroblast), and CK19 (epithelial duct cells) compared to control. This suggests murine IMT001 treatment inhibits pathological signs of liver fibrosis in animal models of the disease.

### Example 40. Gal3 Interacts with TGFBR1 and TGFBR2

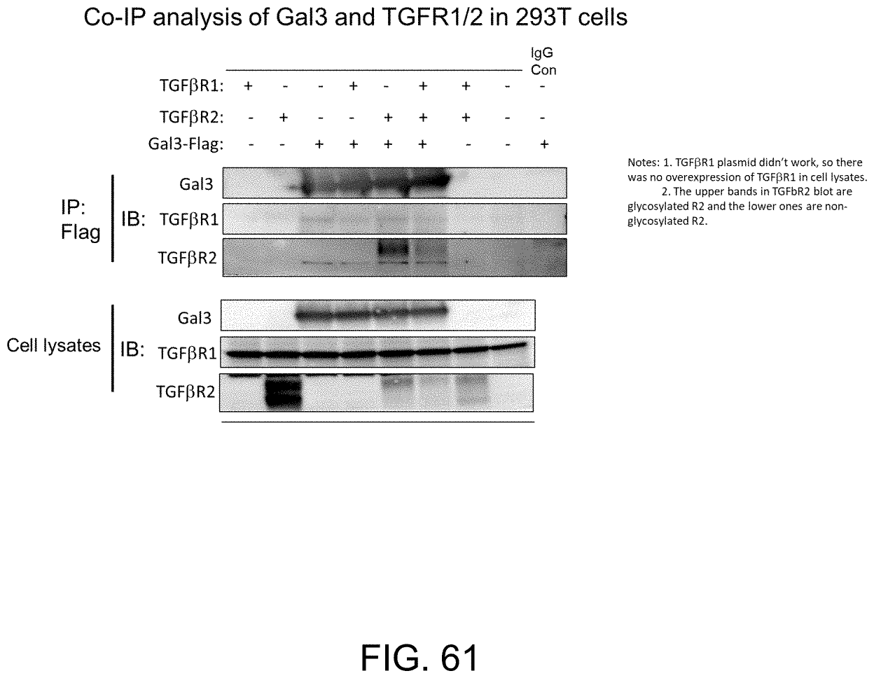

To demonstrate direct interaction between Gal3 with TGFBR1 and TGFBR, HEK 293T cells were transfected with plasmids to express TGFBR1, TGFBR2, and Gal3-Flag alone or in combination as depicted (FIG. 61). The cells were collected 24 hours post-transfection and lysed to be analyzed by anti-Flag immunoprecipitation. Both TGFBR1 and TGFBR2 were detected in the immunoprecipitated samples with high specificity, indicating that they interact with Gal3. Both glycosylated and non-glycosylated TGFBR2 were detected.

### Example 41. Smad3 Phosphorylation is Inhibited by Anti-Gal3 Antibodies

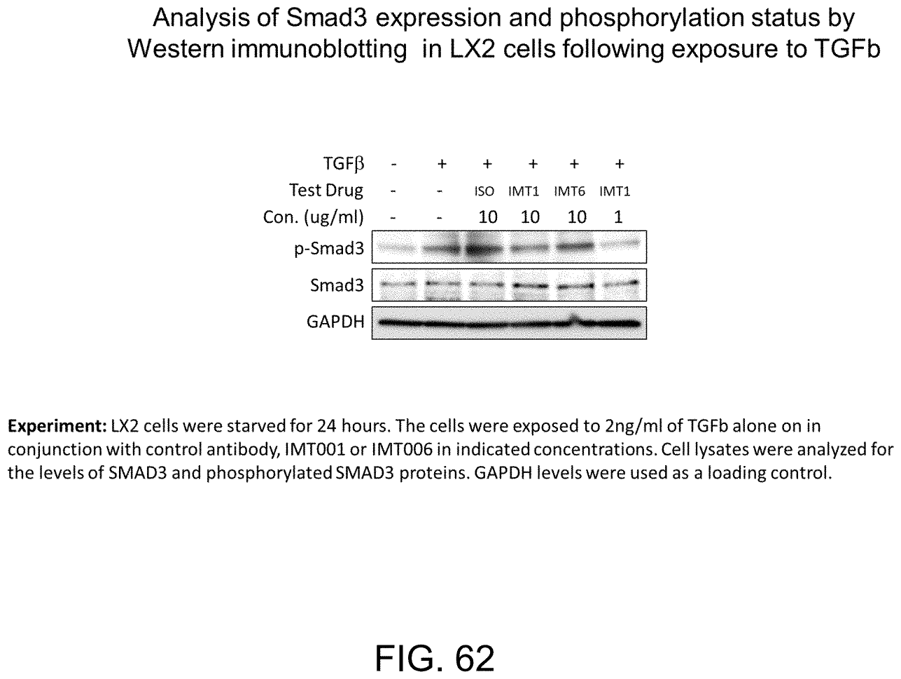

To explore the effect of IMT001 and IMT006 antibodies on the expression of TGF-β downstream signaling, the phosphorylation status of Smad2 and Smad3 was analyzed. For the Smad3 analysis, the LX-2 cells were starved for 24 hours. The cells were then exposed to 2 ng/mL of TGF-β either alone or in conjunction with control isotype antibody, IMT001, or IMT006 at indicated concentrations (FIG. 62). Cell lysates were analyzed for Smad3 expression levels and phosphorylation status. GAPDH was used as a loading control. A reduction in ratio of phosphorylated Smad3 over total Smad3 is observed for the 1 μg/mL and 10 μg/mL IMT001 conditions.

For the Smad2 analysis, NRK-49F (rat kidney fibroblasts) are starved for 24 hours, and then exposed to TGF-β either alone or in conjunction with control isotype antibody (HuIgG4), TB001 (IMT001), or TB006 (IMT006) at the indicated concentrations. Cells are analyzed by immunofluorescence to detect levels and cellular distribution of phosphorylated Smad2 (pSmad2). Both, IMT001 and IMT006 treatment inhibits TGFβ-induced translocation of phosphorylated Smad2 into cell nucleus when compared to isotype control.

### Example 42. Gal3 Antibodies with APP695-Gal3 Blocking Activity Inhibit the Formation of Aβ-42 Fibrils/Oligomers

To assess the activity of Gal3 to induce formation of Aβ-42 oligomers, one mg of the lyophilized Aβ42 peptide (r-peptide) was resuspended in 90 μL of 100 mM NaOH and incubated for 10 minutes. This solution was then diluted to a final concentration of 0.1 mg/mL by adding 10 mM sodium phosphate buffer, pH 7.4. To examine a direct interaction between Gal3 and Aβ, synthetic Aβ42 peptide was mixed with different amounts of human recombinant Gal3 protein and incubated for 5 hours. Different amounts of rhGal3 was added (1, 3, 10, 30 and 100 μg/mL). The solution was continuously stirred during the aggregation time course using a stir bar. The solution was stirred for 5 hours at 37° C. 2 μL of sample was pipetted onto a Whatman nitrocellulose membrane at the appropriate time points (time 0-5 hours) for dot blot and 15 μL of each appropriate time point was frozen down for SDS-PAGE to confirm the low and high molecular weight Aβ42 oligomer formation.

To assess the confirmation of Aβ-42 oligomers following incubation with rGal3, 15 μL of each sample (Aβ42 incubated with different concentration of rhGal3) from time 0, 1, and 5 hour time points were mixed with 4× loading buffer (125 mM Tris, pH 6.8, 4% SDS, 16% glycerol, 10% 2-mercaptoethanol, bromophenol blue), and the resulting 20 μL mixtures were loaded onto a 4-12% Criterion™ XT Bis-Tris Protein Gel. The gel was then run at 100 V in SDS-PAGE apparatus (Biorad), and the resolved proteins were transferred onto a nitrocellulose membrane at 300 mA for 60 min. Nonspecific binding was blocked by incubating the membrane in 10% nonfat dried milk in TBS-T for 1 hour at room temperature. The blot was then incubated in the appropriate secondary antibody (Goat Anti-Mouse IgG H&L (HRP) for 1 hour at room temperature. Following three 5-min washes in TBS-T, the membrane was incubated with chemiluminescent detection reagents for 1-5 seconds.

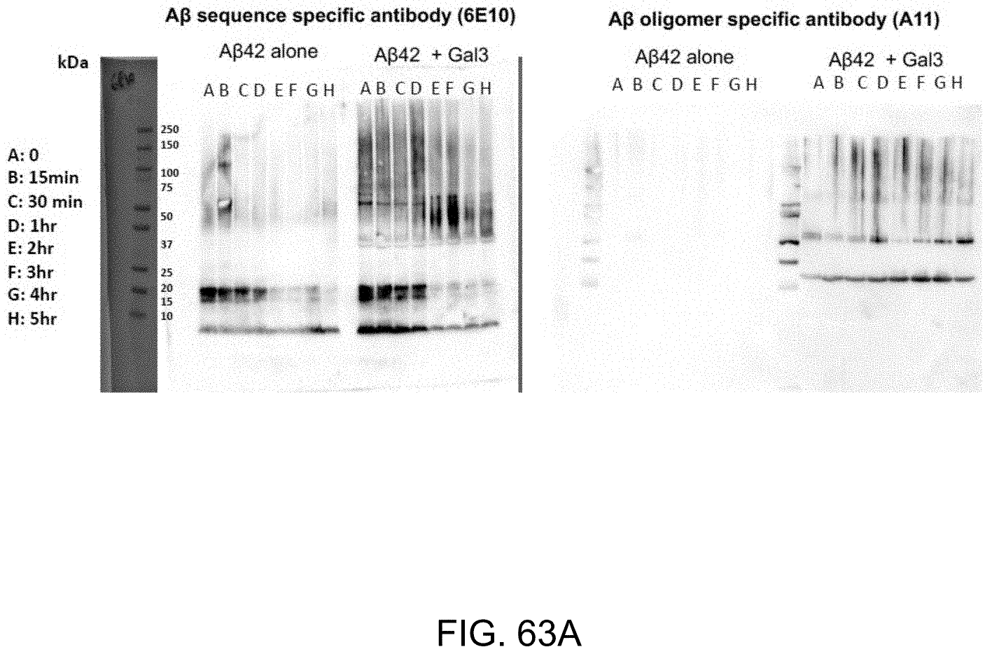

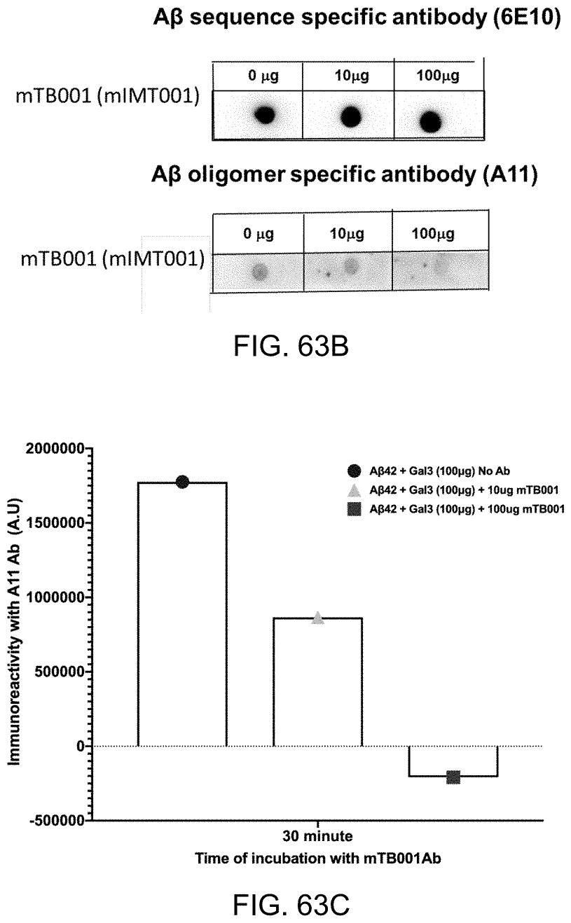

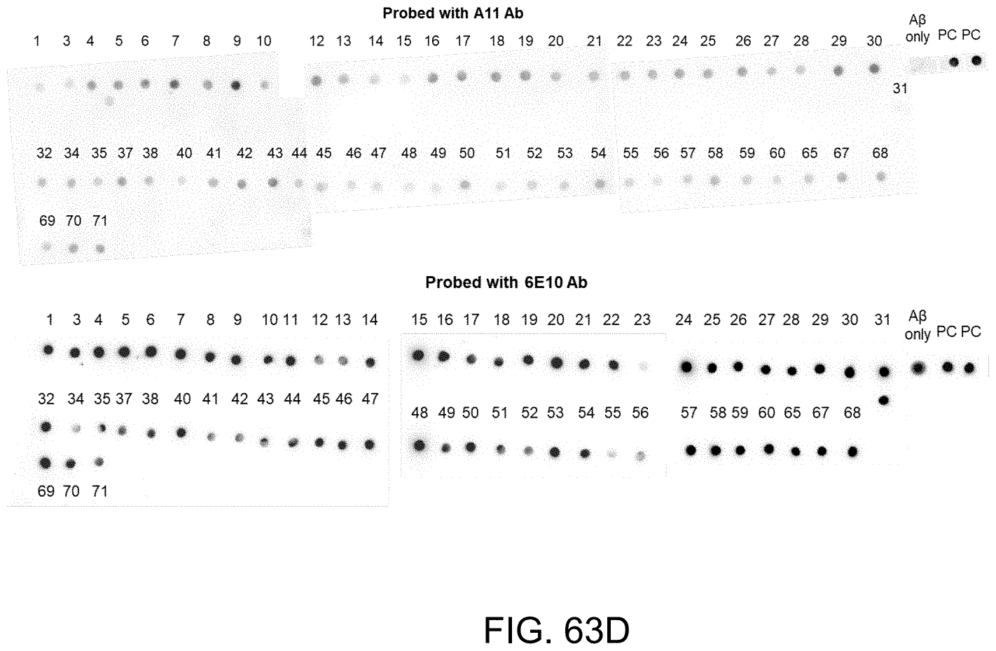

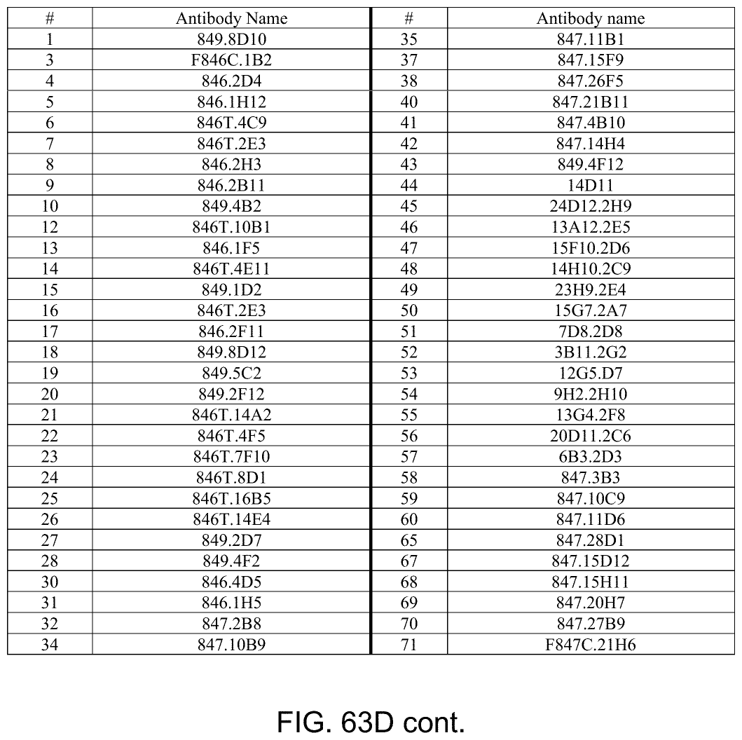

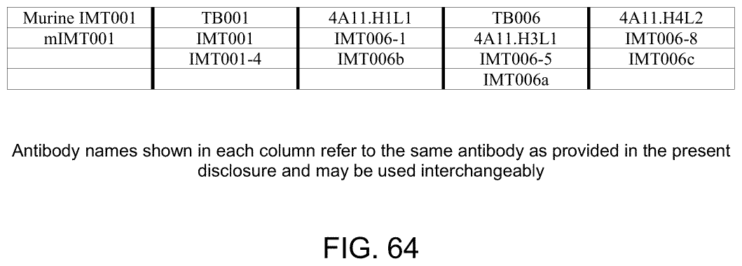

Amyloid-D (Aβ) oligomers largely cascade underlying the pathology of Alzheimer's disease (Aβ). Gal3 promotes Aβ42 aggregation into oligomers (probed with Aβ oligomer specific antibody A11). In order to detect the presence of Aβ42, the membrane was re-probed with Aβ sequence dependent monoclonal antibody 6E10 (Biolegend) (FIG. 63A).

To assess the role of anti-Gal3 antibodies in disrupting Aβ42 oligomers, different concentrations of mTB001 was incubated with Gal3-induced Aβ42 oligomers. It was observed within 30 minutes that Aβ42 oligomers were degraded into monomers as shown in FIG. 63B. Quantification of the dot plot with the Aβ oligomer specific antibody All indicates the concentration-dependent ability of mTB001 in inhibiting Gal3-induced Aβ oligomerization (FIG. 63C).

A large scale screen of anti-Gal3 antibodies disclosed herein was performed to test their ability to disrupt Aβ-42 oligomers. The anti-Gal3 antibodies were incubated with hGal3-65-250 isoform-induced Aβ-42 oligomers. Aβ-42 oligomers were detected with Aβ oligomer specific antibody A11 and total Aβ-42 was detected with 6E10. It was observed within 1 hour that Aβ-42 oligomers were degraded into monomers (FIG. 63D). It was found that the following antibodies exhibited significant inhibition of Aβ-42 oligomer formation: 849.8D10 (1), 846.1B2 (3), 849.4B2 (10), 846T.4E11 (14), 849.1D2 (15), 849.2F12 (20), 846T.14A2 (21), 846T.14E4 (26), 849.2D7 (27), 849.4F2 (28), 847.21B11 (40), 14D11 (44), 24D12.2H9 (45), 13A12.2E5 (46), 15F10.2D6 (47), 14H10.2C9 (48), 23H9.2E4 (49), 7D8.2D8 (51), 12G5.D7 (53), 20D11.2C6 (56), 847.11D6 (60), 847.20H7 (69). Antibody 14D11 has been described in PCT publication WO 2019/152895, hereby expressly incorporated by reference in its entirety.

In summary, Gal3 facilitates the formation of Aβ42 oligomers in a concentration-dependent manner, and anti-Gal3 antibodies (e.g. mTB001) reduces toxic Aβ42 oligomers into monomers.

### Example 43. Identification of Gal3 Region Involved in Interaction with Aβ-42 Peptide/Oligomers

To identify the Gal3 protein region involved in the interaction with Aβ-42 oligomers, one mg of the lyophilized peptide (r-peptide) was resuspended in 90 μL of 100 mM NaOH and incubated for 10 minutes. This solution was then diluted to a final concentration of 0.1 mg/mL by adding 10 mM sodium phosphate buffer, pH 7.4. To examine a direct interaction between different forms of recombinant human Gal3 (rhGal3) and truncated rhGal3 peptides on aggregation of Aβ-42 peptide, synthetic Aβ-42 peptide was mixed with different forms of rhGal3 protein or truncated rhGal3 peptides and incubated for 5 hours. Different forms of rhGal3 and truncated rhGal3 peptides were added at 100 μg/mL. The solution was continuously stirred during the aggregation time course using a stir bar. The solution was stirred for 5 hours at 37° C. 2 μL of each sample was pipetted onto a Whatman nitrocellulose membrane at the appropriate time points (time 0-5 hours) for dot blot to confirm the formation of Aβ-42 oligomers probed with oligomer specific antibody A11. In order to confirm the levels of Aβ-42, the membrane was also probed with Aβ-42 sequence specific antibody 6E10.

Briefly, two (2) μL of each sample was pipetted onto a Whatman nitrocellulose membrane at the appropriate time points (time 0-5 hours). After 5 hours, the dot blot membrane was incubated in 10% nonfat dried milk in TBS-T for 1 hour at room temperature to block the nonspecific binding. The blot was then incubated in the appropriate primary antibody (6E10 and A11) overnight at 4° C. After three 5-minute washes in TBS-T, the membranes were incubated with the appropriate secondary antibody (goat anti-rabbit IgG H&L (HRP) and goat anti-mouse IgG H&L (HRP)) for 1 hour at room temperature. Following three 5-minute washes in TBS-T, the membrane was incubated in chemiluminescent detection reagents for 1-5 seconds. The dot blot images of the results were obtained using imager (Azure Biosystem) and quantified using Licor software and Graph pad prism-8.

The following Gal3 isoforms were tested in the assay: full length Gal3 (labeled as E. coli derived), hGal3-R186S (point mutations for sugar independence), hGal3-P64H (point mutation in the MMP9 cleavage site), rhGal3-65-250 (amino acids 65-250 of Gal3), hGal3-CRD-His (C-terminal domain of Gal3 with histidine tag). All reagents were generated at TrueBinding, Inc.

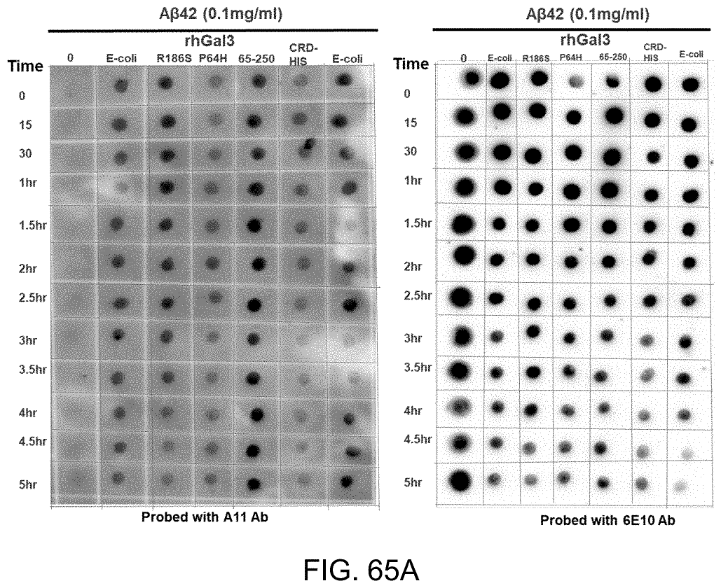

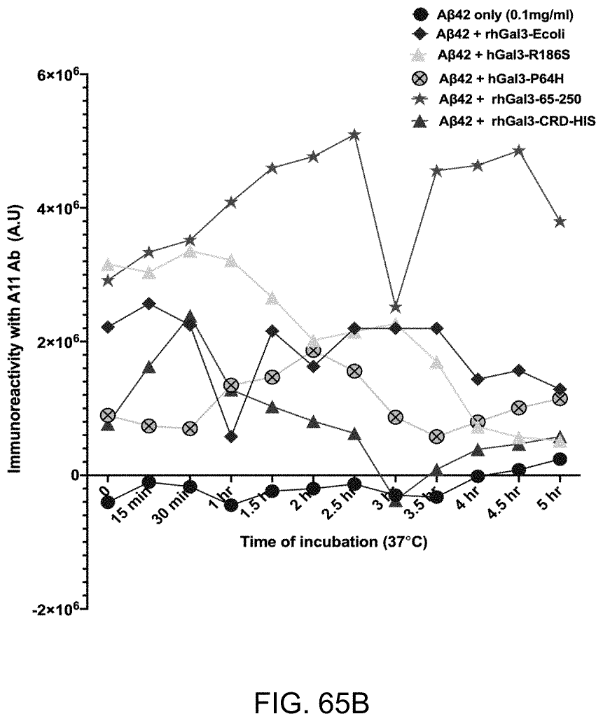

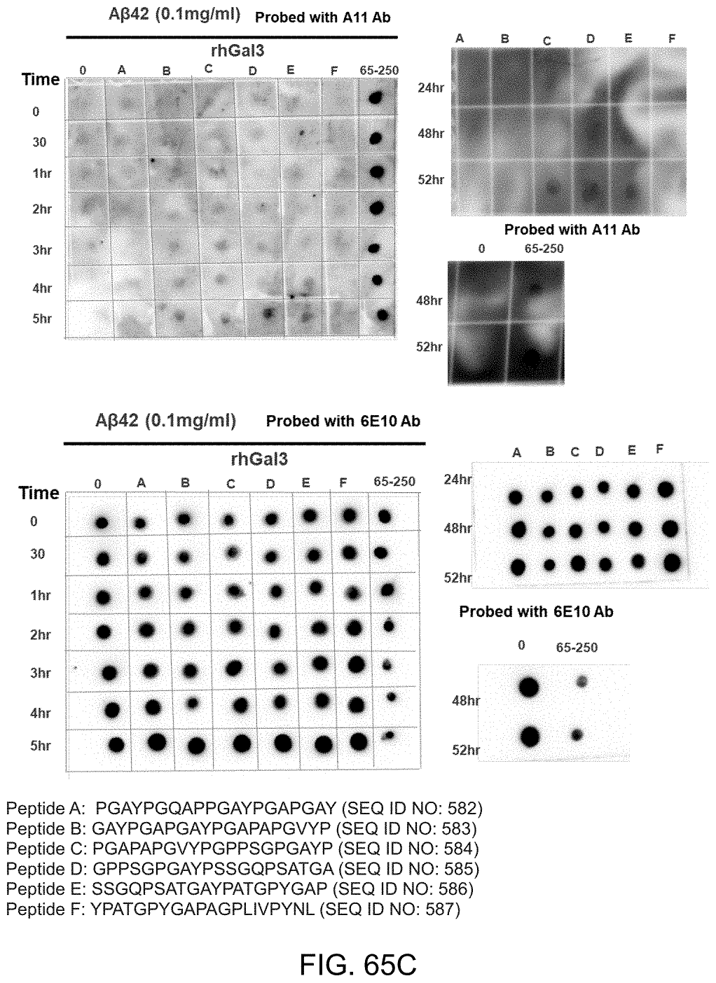

As presented in FIG. 65A, various forms of rhGal3 could promote Aβ-42 aggregation into oligomers. The lane labeled with [0] corresponds to Aβ-42 peptide incubated without any Gal3 isoforms. Quantification of immunoreactivity of Aβ peptide incubated with different forms of rhGal3 with Aβ oligomer specific antibody All by Licor software confirmed that different forms of rhGal3 promotes oligomerization of Aβ-42 peptide. No oligomer formation was observed with the Aβ-42 peptide without rhGal3. The full length Gal3 (labeled E. coli) and hGal3-65-250 were the most efficient Gal3 forms promoting Aβ-42 oligomerization as shown in FIG. 65B.

To further narrow down the region in the Gal3 sequence responsible for interaction with the Aβ-42 peptide, various peptides derived from the rhGal3 sequence were incubated with Aβ-42 oligomers during the time course of incubation. rhGal3 peptides B, C, D, and E (SEQ ID NOs: 583-586) showed time-dependent increase in A11 immunoreactivity as shown in FIG. 65C. rhGal3-65-250 was used as a positive control. The amino acid sequences of overlapping rhGal3 peptides tested is shown in FIG. 65C. Gal3 peptides corresponding to amino acid 71-100 of Gal3 (PGAPAPGVYPGPPSGPGAYPSSGQPSATGA) was sufficient to facilitate the formation of Aβ-42 oligomers in a time-dependent manner.

In some embodiments, any antibody that blocks and/or binds to the above sequence of GAL3 can be used to inhibit or slow oligomerization and aspects related thereto. Thus, in some embodiments, antibodies that bind to, or at least block this sequence can be useful for blocking oligomerization.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-283. (canceled)

284. An antibody conjugate comprising:
an anti-Gal3 antibody or binding fragment thereof; and
a payload conjugated to the anti-Gal3 antibody or binding fragment thereof, wherein the antibody conjugate is able to cross a blood-brain barrier.

285. A method of treating a neurological disorder in a subject in need thereof, comprising:
administering to the subject an effective amount of an anti-Gal3 antibody or binding fragment thereof, thereby treating the neurological order.

286. A method of delivering a payload to the central nervous system of a subject in need thereof, comprising administering to the subject an antibody conjugate comprising an anti-Gal3 antibody or binding fragment thereof and a payload conjugated to the anti-Gal3 antibody or binding fragment thereof, wherein the antibody conjugate is able to cross a blood-brain barrier.

287. A method of increasing the permeability of a payload across the blood-brain barrier of a subject in need thereof, comprising conjugating an anti-Gal3 antibody or binding fragment thereof to the payload to form an antibody conjugate.

288. The method of claim 285, wherein the anti-Gal3 antibody or binding fragment thereof is selected from the group consisting of TB001, TB006, F846C.1B2, F846C.1F5, F846C.1H12, F846C.1H5, F846C.2H3, F846TC.14A2, F846TC.14E4, F846TC.16B5, F846TC.7F10, F847C.10B9, F847C.11B1, F847C.12F12, F847C.26F5, F847C.4B10, F849C.8D10, F849C.8H3, 846.2B11, 846.4D5, 846.2D4, 846.2F11, 846T.10B1, 846T.2E3, 846T.4C9, 846T.4E11, 846T.4F5, 846T.8D1, 847.10C9, 847.11D6, 847.15D12, 847.15F9, 847.15H11, 847.20H7, 847.21B11, 847.27B9, 847.28D1, 847.2B8, 847.3B3, 849.1D2, 849.2D7, 849.2F12, 849.4B2, 849.4F12, 849.4F2, 849.5C2, 849.8D12, F847C.21H6, F846C.1B2, F846C.1F5, F846C.1H12, F846C.2H3, F846TC.14E4, F846TC.16B5, F846TC.7F10, F849C.8D10, 846.4D5, or a binding fragment thereof.

289. The method of claim 285, wherein the proteopathy comprises Alzheimer's disease, cerebral β-amyloid angiopathy, retinal ganglion cell degeneration in glaucoma, Parkinson's disease, Lewy dementia, multiple system atrophy, synucleinopathy, Pick's disease, corticobasal degeneration, taupathy, frontotemporal lobar degeneration, Huntington's disease, dentatorubropallidoluysian atrophy, spinal and bulbal muscular atrophy, spinocerebellar ataxia, fragile X syndrome, Baratela-Scott syndrome, Freidrich's ataxia, myotonic dystrophy, Alexander disease, familial British dementia, familial Danish dementia, Palizaeus-Merzbacher disease, seipinopathy, AA (secondary) amyloidosis, type II diabetes, fibrinogen amyloidosis, dialysis amyloidosis, inclusion body myositis/myopathy, familial amyloidotic neuropathy, senile systemic amyloidosis, serpinopathy, cardiac atrial amyloidosis, pituitary prolactinoma, insulin amyloidosis, corneal lactoferrin amyloidosis, pulmonary alveolar proteinosis, seminal vesicle amyloid, cutaneous lichen amyloidosis, Mallory bodies, or odontogenic (Pindborg) tumor amyloid, or any disease caused by the misfolding or aggregation of proteins, or any combination thereof.

290. The method of claim 285, wherein the anti-Gal3 antibody or binding fragment thereof promotes phagocytic function of microglia in the subject.

291. The method of claim 285, wherein the anti-Gal3 antibody or binding fragment thereof decreases phospho-Tau levels or Gal3 levels, or both, in the brain of the subject.

292. The method of claim 285, wherein the anti-Gal3 antibody or binding fragment thereof inhibits AP-mediated activation of microglia in the subject by at least 50%.

293. The method of claim 285, wherein the anti-Gal3 antibody or binding fragment thereof inhibits Aβ fibril or oligomer formation in the subject by at least 50%.

294. The method of claim 285, wherein the anti-Gal3 antibody or binding fragment thereof promotes neuronal regeneration in the subject.

295. The method of claim 285, wherein the anti-Gal3 antibody or binding fragment thereof disrupts binding between Gal3 and Toll-like receptor 4 (TLR4) or triggering receptor expressed on myeloid cells 2 (TREM2), or both.

296. The method of claim 12, wherein the binding between Gal3 and TLR4 or TREM2, or both, is disrupted by at least 50%.

297. The method of claim 285, wherein the anti-Gal3 antibody or binding fragment thereof is administered enterally, orally, intranasally, parenterally, intracranially, subcutaneously, intramuscularly, intradermally, or intravenously, or any combination thereof.

298. The method of claim 285, wherein conjugation of the payload to the anti-Gal3 antibody or binding fragment thereof increases the permeability of the payload across the blood-brain barrier by at least 5%, compared to the unconjugated payload.

299. The method of claim 285, wherein the permeability of the payload across the blood-brain barrier is less than 95% of the permeability of the antibody conjugate across the blood-brain barrier.

300. The method of claim 285, wherein the payload or the anti-Gal3 antibody or binding fragment thereof, or both, is used to treat a neurological disorder that is treated in the brain.

301. The method of claim 285, wherein the payload is a cytotoxic payload, microtubule disrupting agent, DNA modifying agent, Akt inhibitor, polymerase inhibitor, detectable moiety, immunomodulatory agent, immune modulator, immunotoxin, nucleic acid polymer, aptamer, peptide, protein, enzyme, second antibody or any combination thereof.

302. The method of claim 285, wherein the payload does not normally cross the blood-brain barrier.

303. The method of claim 285, wherein the antibody or binding fragment is administered as a supplement to PD1/PDL1 blockade therapies and/or a CTLA4 blockade therapy.

304. The method of claim 303, wherein the PD1/PDL1 blockade therapies comprise pembrolizumab, nivolumab, cemiplimab, spartalizumab, camrelizumab, sintilimab, tislelizumab, toripalimab, AMP-224, AMP-514, atezolizumab, avelumab, durvalumab, KN035, CK-301, AUNP12, CA-170, BMS-986189, ipilimumab, and/or tremilimumab.

305. The method of claim 285, wherein the antibody or binding fragment thereof binds to Gal3 with a dissociation constant (KD) of less than 1 nM, less than 1.2 nM, less than 2 nM, less than 5 nM.

306. The method of claim 285, wherein the antibody or binding fragment thereof comprises a monovalent Fab′, a divalent Fab2, a single-chain variable fragment (scFv), a diabody, a minibody, a nanobody, a single-domain antibody (sdAb), a camelid antibody, a bispecific antibody, a full length antibody, a humanized antibody, or binding fragment thereof.

307. The method of claim 285, wherein the antibody or binding fragment thereof comprises an IgG1, IgG2, or IgG4 framework.

308. The method of claim 285, wherein the antibody or binding fragment thereof retards brain tumor growth.

309. The method of claim 285, wherein the blood-brain barrier is a mammalian blood-brain barrier.

310. The method of claim 285, wherein the blood-brain barrier is a human blood-brain barrier.

311. The method of claim 285, wherein the antibody or binding fragment thereof disrupts an interaction between galectin-3 (Gal3) and a transforming growth factor beta (TGF-b) receptor.

312. The method of claim 285, wherein the TGF-b receptor is expressed by a cell.

313. The method of claim 285, wherein the antibody comprises (1) a heavy chain variable region comprising a V_{H}-CDR1, a V_{H}-CDR2, and a V_{H}-CDR3; and (2) a light chain variable region comprising a V_{L}-CDR1, a V_{L}-CDR2, and a V_{L}-CDR3, wherein
the V_{H}-CDR1 comprises an amino acid sequence having at least 90% identity to SEQ ID NO: 31;
the V_{H}-CDR2 comprises an amino acid sequence having at least 90% identity to SEQ ID NO: 72;
the V_{H}-CDR3 comprises an amino acid sequence having at least 90% identity to SEQ ID NO: 113;
the V_{H}-CDR1 comprises an amino acid sequence having at least 90% identity to SEQ ID NO: 170;
the V_{H}-CDR1 comprises an amino acid sequence having at least 90% identity to SEQ ID NO: 221; and
the V_{H}-CDR1 comprises an amino acid sequence having at least 90%, to SEQ ID NO: 248.


SEQUENCE LISTING (867 sequences)

SEQ ID NO: 1 - protein, 250 aa, Homo sapiens [sequence omitted]

SEQ ID NO: 2 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - protein, 20 aa, synthetic construct
  REGION 1..20: note = Gal3 Peptide 1
ADNFSLHDALSGSGNPNPQG

SEQ ID NO: 4 - protein, 20 aa, synthetic construct
  REGION 1..20: note = Gal3 Peptide 2
SGSGNPNPQGWPGAWGNQPA

SEQ ID NO: 5 - protein, 20 aa, synthetic construct
  REGION 1..20: note = Gal3 Peptide 3
WPGAWGNQPAGAGGYPGASY

SEQ ID NO: 6 - protein, 20 aa, synthetic construct
  REGION 1..20: note = Gal3 Peptide 4
GAGGYPGASYPGAYPGQAPP

SEQ ID NO: 7 - protein, 20 aa, synthetic construct
  REGION 1..20: note = Gal3 Peptide 5
PGAYPGQAPPGAYPGQAPPG

SEQ ID NO: 8 - protein, 20 aa, synthetic construct
  REGION 1..20: note = Gal3 Peptide 6
GAYPGQAPPGAYPGAPGAYP

SEQ ID NO: 9 - protein, 20 aa, synthetic construct
  REGION 1..20: note = Gal3 Peptide 7
AYPGAPGAYPGAPAPGVYPG

SEQ ID NO: 10 - protein, 20 aa, synthetic construct
  REGION 1..20: note = Gal3 Peptide 8
GAPAPGVYPGPPSGPGAYPS

SEQ ID NO: 11 - protein, 20 aa, synthetic construct
  REGION 1..20: note = Gal3 Peptide 9
PPSGPGAYPSSGQPSATGAY

SEQ ID NO: 12 - protein, 20 aa, synthetic construct
  REGION 1..20: note = Gal3 Peptide 10
SGQPSATGAYPATGPYGAPA

SEQ ID NO: 13 - protein, 20 aa, synthetic construct
  REGION 1..20: note = Gal3 Peptide 11
PATGPYGAPAGPLIVPYNLP

SEQ ID NO: 14 - protein, 20 aa, synthetic construct
  REGION 1..20: note = Gal3 Peptide 12
GPLIVPYNLPLPGGVVPRML

SEQ ID NO: 15 - protein, 20 aa, synthetic construct
  REGION 1..20: note = Gal3 Peptide 13
LPGGVVPRMLITILGTVKPN

SEQ ID NO: 16 - protein, 20 aa, synthetic construct
  REGION 1..20: note = Gal3 Peptide 14
ITILGTVKPNANRIALDFQR

SEQ ID NO: 17 - protein, 20 aa, synthetic construct
  REGION 1..20: note = Gal3 Peptide 15
ANRIALDFQRGNDVAFHFNP

SEQ ID NO: 18 - protein, 20 aa, synthetic construct
  REGION 1..20: note = Gal3 Peptide 16
GNDVAFHFNPRFNENNRRVI

SEQ ID NO: 19 - protein, 20 aa, synthetic construct
  REGION 1..20: note = Gal3 Peptide 17
RFNENNRRVIVCNTKLDNNW

SEQ ID NO: 20 - protein, 20 aa, synthetic construct
  REGION 1..20: note = Gal3 Peptide 18
VCNTKLDNNWGREERQSVFP

SEQ ID NO: 21 - protein, 20 aa, synthetic construct
  REGION 1..20: note = Gal3 Peptide 19
GREERQSVFPFESGKPFKIQ

SEQ ID NO: 22 - protein, 20 aa, synthetic construct
  REGION 1..20: note = Gal3 Peptide 20
FESGKPFKIQVLVEPDHFKV

SEQ ID NO: 23 - protein, 20 aa, synthetic construct
  REGION 1..20: note = Gal3 Peptide 21
VLVEPDHFKVAVNDAHLLQY

SEQ ID NO: 24 - protein, 20 aa, synthetic construct
  REGION 1..20: note = Gal3 Peptide 22
AVNDAHLLQYNHRVKKLNEI

SEQ ID NO: 25 - protein, 20 aa, synthetic construct
  REGION 1..20: note = Gal3 Peptide 23
NHRVKKLNEISKLGISGDID

SEQ ID NO: 26 - protein, 19 aa, synthetic construct
  REGION 1..19: note = Gal3 Peptide 24
SKLGISGDIDLTSASYTMI

SEQ ID NO: 27 - protein, 7 aa, synthetic construct
  REGION 1..7: note = VH_CDR1_1
GYTFTNY

SEQ ID NO: 28 - protein, 7 aa, synthetic construct
  REGION 1..7: note = VH_CDR1_2
GYSFTNY

SEQ ID NO: 29 - protein, 7 aa, synthetic construct
  REGION 1..7: note = VH_CDR1_3
GYTFTDY

SEQ ID NO: 30 - protein, 7 aa, synthetic construct
  REGION 1..7: note = VH_CDR1_4
GYKFKTY

SEQ ID NO: 31 - protein, 7 aa, synthetic construct
  REGION 1..7: note = VH_CDR1_5
GYTFTTY

SEQ ID NO: 32 - protein, 7 aa, synthetic construct
  REGION 1..7: note = VH_CDR1_6
GFAFSSY

SEQ ID NO: 33 - protein, 7 aa, synthetic construct
  REGION 1..7: note = VH_CDR1_7
GYTFTDF

SEQ ID NO: 34 - protein, 7 aa, synthetic construct
  REGION 1..7: note = VH_CDR1_8
GYAFTTY

SEQ ID NO: 35 - protein, 7 aa, synthetic construct
  REGION 1..7: note = VH_CDR1_9
GFTFSSY

SEQ ID NO: 36 - protein, 8 aa, synthetic construct
  REGION 1..8: note = VH_CDR1_10
GYTFTNYG

SEQ ID NO: 37 - protein, 8 aa, synthetic construct
  REGION 1..8: note = VH_CDR1_11
GYTFTNFG

SEQ ID NO: 38 - protein, 8 aa, synthetic construct
  REGION 1..8: note = VH_CDR1_12
GFTFSSYA

SEQ ID NO: 39 - protein, 10 aa, synthetic construct
  REGION 1..10: note = VH_CDR1_13
GFSLSTSGMG

SEQ ID NO: 40 - protein, 8 aa, synthetic construct
  REGION 1..8: note = VH_CDR1_14
GFTFSNYG

SEQ ID NO: 41 - protein, 8 aa, synthetic construct
  REGION 1..8: note = VH_CDR1_15
GYIFTNYG

SEQ ID NO: 42 - protein, 8 aa, synthetic construct
  REGION 1..8: note = VH_CDR1_16
GNTFTDHN

SEQ ID NO: 43 - protein, 8 aa, synthetic construct
  REGION 1..8: note = VH_CDR1_17
GYTFTSYW

SEQ ID NO: 44 - protein, 10 aa, synthetic construct
  REGION 1..10: note = VH_CDR1_18
GFSLSTFGMG

SEQ ID NO: 45 - protein, 6 aa, synthetic construct
  REGION 1..6: note = VH_CDR2_1
NTNTGE

SEQ ID NO: 46 - protein, 8 aa, synthetic construct
  REGION 1..8: note = VH_CDR2_2
IYPGSGNT

SEQ ID NO: 47 - protein, 8 aa, synthetic construct
  REGION 1..8: note = VH_CDR2_3
IYPGTGNT

SEQ ID NO: 48 - protein, 8 aa, synthetic construct
  REGION 1..8: note = VH_CDR2_4
INTYSGVP

SEQ ID NO: 49 - protein, 8 aa, synthetic construct
  REGION 1..8: note = VH_CDR2_5
ISDGGVYT

SEQ ID NO: 50 - protein, 8 aa, synthetic construct
  REGION 1..8: note = VH_CDR2_6
INPKNGGI

SEQ ID NO: 51 - protein, 8 aa, synthetic construct
  REGION 1..8: note = VH_CDR2_7
VNTYSGVP

SEQ ID NO: 52 - protein, 8 aa, synthetic construct
  REGION 1..8: note = VH_CDR2_8
IYPGSNDT

SEQ ID NO: 53 - protein, 8 aa, synthetic construct
  REGION 1..8: note = VH_CDR2_9
ISDGGIYT

SEQ ID NO: 54 - protein, 8 aa, synthetic construct
  REGION 1..8: note = VH_CDR2_10
INTYTGEP

SEQ ID NO: 55 - protein, 7 aa, synthetic construct
  REGION 1..7: note = VH_CDR2_11
ISSGGDT

SEQ ID NO: 56 - protein, 7 aa, synthetic construct
  REGION 1..7: note = VH_CDR2_12
IWWDDDK

SEQ ID NO: 57 - protein, 8 aa, synthetic construct
  REGION 1..8: note = VH_CDR2_13
ISSGGSYT

SEQ ID NO: 58 - protein, 8 aa, synthetic construct
  REGION 1..8: note = VH_CDR2_14
IYPYNGGT

SEQ ID NO: 59 - protein, 7 aa, synthetic construct
  REGION 1..7: note = VH_CDR2_15
ISSGGST

SEQ ID NO: 60 - protein, 8 aa, synthetic construct
  REGION 1..8: note = VH_CDR2_16
IYPGSSSI

SEQ ID NO: 61 - protein, 9 aa, synthetic construct
  REGION 1..9: note = VH_CDR3_1
APYDNFFAY

SEQ ID NO: 62 - protein, 9 aa, synthetic construct
  REGION 1..9: note = VH_CDR3_2
STAPGGFDV

SEQ ID NO: 63 - protein, 15 aa, synthetic construct
  REGION 1..15: note = VH_CDR3_3
ARFAYYYGSGGYFDY

SEQ ID NO: 64 - protein, 12 aa, synthetic construct
  REGION 1..12: note = VH_CDR3_4
ARDGNYGDPMDY

SEQ ID NO: 65 - protein, 10 aa, synthetic construct
  REGION 1..10: note = VH_CDR3_5
STPYEYDGAY

SEQ ID NO: 66 - protein, 6 aa, synthetic construct
  REGION 1..6: note = VH_CDR3_6
VRDGGY

SEQ ID NO: 67 - protein, 9 aa, synthetic construct
  REGION 1..9: note = VH_CDR3_7
ARGPYAMDY

SEQ ID NO: 68 - protein, 8 aa, synthetic construct
  REGION 1..8: note = VH_CDR3_8
TSGYGFPY

SEQ ID NO: 69 - protein, 13 aa, synthetic construct
  REGION 1..13: note = VH_CDR3_9
ANYFGCSGWFFDV

SEQ ID NO: 70 - protein, 15 aa, synthetic construct
  REGION 1..15: note = VH_CDR3_10
ARWGGYAGDYYAMDF

SEQ ID NO: 71 - protein, 15 aa, synthetic construct
  REGION 1..15: note = VH_CDR3_11
ARWGGYDGDYYAMDY

SEQ ID NO: 72 - protein, 15 aa, synthetic construct
  REGION 1..15: note = VH_CDR3_12
ARWGGYAGDYYAMDY

SEQ ID NO: 73 - protein, 10 aa, synthetic construct
  REGION 1..10: note = VH_CDR3_13
ASYGNSLFDY

SEQ ID NO: 74 - protein, 7 aa, synthetic construct
  REGION 1..7: note = VH_CDR3_14
VRYTMDY

SEQ ID NO: 75 - protein, 16 aa, synthetic construct
  REGION 1..16: note = VH_CDR3_15
ARNLYDGSYGYYAMDY

SEQ ID NO: 76 - protein, 16 aa, synthetic construct
  REGION 1..16: note = VH_CDR3_16
ARHAHYYGVSPYYFDY

SEQ ID NO: 77 - protein, 12 aa, synthetic construct
  REGION 1..12: note = VH_CDR3_17
AKFGNYVGAMDY

SEQ ID NO: 78 - protein, 13 aa, synthetic construct
  REGION 1..13: note = VH_CDR3_18
ARGEYDYLAWFAY

SEQ ID NO: 79 - protein, 11 aa, synthetic construct
  REGION 1..11: note = VH_CDR3_19
TRFTTVVEMDY

SEQ ID NO: 80 - protein, 5 aa, synthetic construct
  REGION 1..5: note = VH_CDR3_20
ARWGY

SEQ ID NO: 81 - protein, 13 aa, synthetic construct
  REGION 1..13: note = VH_CDR3_21
ARIDYGDYVGFAY

SEQ ID NO: 82 - protein, 16 aa, synthetic construct
  REGION 1..16: note = VL_CDR1_1
RSSKSLLYKDGKTYLN

SEQ ID NO: 83 - protein, 11 aa, synthetic construct
  REGION 1..11: note = VL_CDR1_2
KSLLHSDGITY

SEQ ID NO: 84 - protein, 6 aa, synthetic construct
  REGION 1..6: note = VL_CDR1_3
QGINSN

SEQ ID NO: 85 - protein, 11 aa, synthetic construct
  REGION 1..11: note = VL_CDR1_4
QSLLYTNGKTY

SEQ ID NO: 86 - protein, 11 aa, synthetic construct
  REGION 1..11: note = VL_CDR1_5
QSLFDSDGKTY

SEQ ID NO: 87 - protein, 11 aa, synthetic construct
  REGION 1..11: note = VL_CDR1_6
QSIVHNNGNTY

SEQ ID NO: 88 - protein, 6 aa, synthetic construct
  REGION 1..6: note = VL_CDR1_7
QGINNY

SEQ ID NO: 89 - protein, 6 aa, synthetic construct
  REGION 1..6: note = VL_CDR1_8
QNIYIW

SEQ ID NO: 90 - protein, 6 aa, synthetic construct
  REGION 1..6: note = VL_CDR1_9
QDISNY

SEQ ID NO: 91 - protein, 11 aa, synthetic construct
  REGION 1..11: note = VL_CDR1_10
QSIVHSNGNTY

SEQ ID NO: 92 - protein, 12 aa, synthetic construct
  REGION 1..12: note = VL_CDR1_11
QSVLYSSNQKNY

SEQ ID NO: 93 - protein, 12 aa, synthetic construct
  REGION 1..12: note = VL_CDR1_12
QSVLYSSSQKNY

SEQ ID NO: 94 - protein, 11 aa, synthetic construct
  REGION 1..11: note = VL_CDR1_13
QSLVHNSGNTY

SEQ ID NO: 95 - protein, 9 aa, synthetic construct
  REGION 1..9: note = VL_CDR1_14
TGAVTTSNY

SEQ ID NO: 96 - protein, 11 aa, synthetic construct
  REGION 1..11: note = VL_CDR1_15
KSLLHTNGITF

SEQ ID NO: 97 - protein, 12 aa, synthetic construct
  REGION 1..12: note = VL_CDR1_16
QSLLNSSNQKNY

SEQ ID NO: 98 - protein, 6 aa, synthetic construct
  REGION 1..6: note = VL_CDR1_17
QNINVW

SEQ ID NO: 99 - protein, 6 aa, synthetic construct
  REGION 1..6: note = VL_CDR1_18
IDIDDD

SEQ ID NO: 100 - protein, 11 aa, synthetic construct
  REGION 1..11: note = VL_CDR1_19
QDIVHSSGNTY

SEQ ID NO: 101 - protein, 11 aa, synthetic construct
  REGION 1..11: note = VL_CDR1_20
KSLLYSNGITY

SEQ ID NO: 102 - protein, 7 aa, synthetic construct
  REGION 1..7: note = VL_CDR2_1
LMSTHAS

SEQ ID NO: 103-116 - skipped (14 entries)

SEQ ID NO: 117 - protein, 9 aa, synthetic construct
  REGION 1..9: note = VL_CDR3_1
QQLVDYPLT

SEQ ID NO: 118 - protein, 9 aa, synthetic construct
  REGION 1..9: note = VL_CDR3_2
AQMLEFPLT

SEQ ID NO: 119 - protein, 9 aa, synthetic construct
  REGION 1..9: note = VL_CDR3_3
VQYAQFPPT

SEQ ID NO: 120 - protein, 9 aa, synthetic construct
  REGION 1..9: note = VL_CDR3_4
LQSTHFPLT

SEQ ID NO: 121 - protein, 9 aa, synthetic construct
  REGION 1..9: note = VL_CDR3_5
WQGTHFPLT

SEQ ID NO: 122 - protein, 9 aa, synthetic construct
  REGION 1..9: note = VL_CDR3_6
FQGSHVPLT

SEQ ID NO: 123 - protein, 9 aa, synthetic construct
  REGION 1..9: note = VL_CDR3_7
QQYSQVPYT

SEQ ID NO: 124 - protein, 9 aa, synthetic construct
  REGION 1..9: note = VL_CDR3_8
LQGQSYPLT

SEQ ID NO: 125 - protein, 9 aa, synthetic construct
  REGION 1..9: note = VL_CDR3_9
QQYSKLPYT

SEQ ID NO: 126 - protein, 9 aa, synthetic construct
  REGION 1..9: note = VL_CDR3_10
AQMIEFPLT

SEQ ID NO: 127 - protein, 8 aa, synthetic construct
  REGION 1..8: note = VL_CDR3_11
HQYLSSLT

SEQ ID NO: 128 - protein, 9 aa, synthetic construct
  REGION 1..9: note = VL_CDR3_12
SQSTHVPPT

SEQ ID NO: 129 - protein, 9 aa, synthetic construct
  REGION 1..9: note = VL_CDR3_13
ALWYSNHWV

SEQ ID NO: 130 - protein, 9 aa, synthetic construct
  REGION 1..9: note = VL_CDR3_14
AQNLELPWT

SEQ ID NO: 131 - protein, 9 aa, synthetic construct
  REGION 1..9: note = VL_CDR3_15
QQHYSTPYT

SEQ ID NO: 132 - protein, 9 aa, synthetic construct
  REGION 1..9: note = VL_CDR3_16
QQGQSYPYT

SEQ ID NO: 133 - protein, 9 aa, synthetic construct
  REGION 1..9: note = VL_CDR3_17
LQSDNKPLT

SEQ ID NO: 134 - protein, 9 aa, synthetic construct
  REGION 1..9: note = VL_CDR3_18
ALWYSNHLV

SEQ ID NO: 135 - protein, 9 aa, synthetic construct
  REGION 1..9: note = VL_CDR3_19
FQGSHVPPT

SEQ ID NO: 136 - protein, 116 aa, synthetic construct
  REGION 1..116: note = VH-1
QVQLVQSGSELKKPGASVKVSCKASGYTFTNYGMNWVRQAPGQGLKWMGWINTNTGEPTYVEEFTGRFVFSLETSVSTAYLQISSLKAEDTAVYFCAPYDNFFAYWGQGTTVTVSS

SEQ ID NO: 137 - protein, 116 aa, synthetic construct
  REGION 1..116: note = VH-2
QVQLVQSGAEVKKPGASVKVSCKASGYSFTNYYMHWVRQAPGQRLEWMGWIYPGSGNTNYNEKFQGRVTITADTSASTAYMELSSLRSEDTAVYYCSTAPGGFDVWGQGTTVTVSS

SEQ ID NO: 138 - protein, 122 aa, synthetic construct
  REGION 1..122: note = VH-3
QVQLKQSGAELVRPGASVKLSCKASGYTFTDYYINWVKQRPGQGLEWIARIYPGTGNTDYNEKFKGRATLTAEKSSSTAYMQLSSLTSEDSAVYFCARFAYYYGSGGYFDYWGHGTTLTVSS

SEQ ID NO: 139 - protein, 119 aa, synthetic construct
  REGION 1..119: note = VH-4
QIQLVQSGPELKKPGETVKISCKTSGYKFKTYVMSWVKQAPGKALKWMGWINTYSGVPTYADDFKGRFAFSLETSASTAYLEIINLKNEDTATYFCARDGNYGDPMDYWGQGTSVTVSS

SEQ ID NO: 140 - protein, 117 aa, synthetic construct
  REGION 1..117: note = VH-5
QIQLVQSGPELKKPGETVKISCKASGYTFTTYGMGWVKQAPGKDLKWMGWINTYSGVPTYADDFKGRFAFSLETSASTAYLQISNLKNEDTATYFCSTPYEYDGAYWGQGTLVTVSA

SEQ ID NO: 141 - protein, 113 aa, synthetic construct
  REGION 1..113: note = VH-6
EVQLVESGGGLVKPGGSLKLSCAASGFAFSSYAMSWVRQTPEKRLEWVATISDGGVYTYYTDHVKGRFTISRDNAEDNLYLQMSHLKSEDTAMYYCVRDGGYWGQGTTLTVSS

SEQ ID NO: 142 - protein, 116 aa, synthetic construct
  REGION 1..116: note = VH-7
QIQLVQSGPELKKPGETVKISCKASGYTFTTYAMSWVKQAPGKGLKWMGWINTYSGVPTYADDLKGRFAFSLETSASTAYLQINNLKNEDTATYFCARGPYAMDYWGQGTSVTVSS

SEQ ID NO: 143 - protein, 115 aa, synthetic construct
  REGION 1..115: note = VH-8
EVQLQQSGPELVKPGASVKISCKASGYTFTDFYINWVKQSHGKSLEWIGDINPKNGGINYNPKFKIKATLTVDKSSSTSYMDLRGLTSEDSAVYYCTSGYGFPYWGQGTLVTVSA

SEQ ID NO: 144 - protein, 116 aa, synthetic construct
  REGION 1..116: note = VH-9
QIQLVQSGPELKKPGESVKISCKASGYAFTTYGMSWVQQAPGKGLKWMGWVNTYSGVPTCADDFKGRFAFSLETSASTAYLQINNLRNEDTATYFCARGPYAMDYWGQGTSVTVSS

SEQ ID NO: 145 - protein, 120 aa, synthetic construct
  REGION 1..120: note = VH-10
QVQLQQSGPELVKPGASVKISCKASGYTFTDYYINWVKQRPGQGLEWIGWIYPGSNDTKYNEKFKGKATLTVDTSSSTAYMQLGSLTSEDSAVYFCANYFGCSGWFFDVWGTGTTVTVSS

SEQ ID NO: 146 - protein, 113 aa, synthetic construct
  REGION 1..113: note = VH-11
EVQLVESGGGLVKPGGSLKLSCAASGFTFSSYAMSWVRQTPEKRLEWVATISDGGIYTYYPDNVKGRFTISRDNAKNNLFLQMSHLKSEDTAMYYCVRDGGYWGQGTTLTVSS

SEQ ID NO: 147 - protein, 122 aa, synthetic construct
  REGION 1..122: note = VH-12
QIQLVQSGPELKKPGETVKISCKASGYTFTNYGMNWVKQAPGKGLKWMGWINTYTGEPSYADDFKGRFAFSLETSASTAYLQINNLKNEDMATYFCARWGGYAGDYYAMDFWGQGTSVTVSS

SEQ ID NO: 148 - protein, 122 aa, synthetic construct
  REGION 1..122: note = VH-13
QIQLVQSGPELKKPGETVKISCKASGYTFTNYGMNWVKQAPGKGLKWMGWINTYTGEPSYADDFKGRFAFSLETSASTAYLQINNLKNEDMATYFCARWGGYDGDYYAMDYWGQGTSVTVSS

SEQ ID NO: 149 - protein, 122 aa, synthetic construct
  REGION 1..122: note = VH-14
QIQLVQSGPELKKPGETVKISCKASGYTFTNFGMNWVKQAPGKGLKWMGWINTYTGEPSYADDFKGRFAFSLETSASTAYLQINNLKNEDIATYFCARWGGYAGDYYAMDYWGQGTSVTVSS

SEQ ID NO: 150 - protein, 116 aa, synthetic construct
  REGION 1..116: note = VH-15
EVQLLESGGGLVKPGGSLKLSCAASGFTFSSYAMSWVRQTPEKRLEWVASISSGGDTYYPDSVKGRFTISRDNARNILYLQMSSLRSEDTAMYYCASYGNSLFDYWGQGTTLTVSS

SEQ ID NO: 151 - protein, 114 aa, synthetic construct
  REGION 1..114: note = VH-16
QIQLVQSGPELKKPGETVKISCKASGYTFTNYGMNWVKQAPGKGLKWMGWINTYTGEPTYAGDLKGRFAFSLETSASTAYLQINNLKNEDTATYFCVRYTMDYWGQGTSVTVSS

SEQ ID NO: 152 - protein, 124 aa, synthetic construct
  REGION 1..124: note = VH-17
QVTLKESGPGILQPSQTLSLTCSFSGFSLSTSGMGVGWIRQPSGKGLEWLAHIWWDDDKYYNTALKSGLTISKDASKNQVFLKIASMDTADTATYYCARNLYDGSYGYYAMDYWGQGTSVTVSS

SEQ ID NO: 153 - protein, 122 aa, synthetic construct
  REGION 1..122: note = VH-18
QIQLVQSGPELKKPGETVKISCKASGYTFTNYGMNWVKQAPGKGLKWMGWINTYTGEPAYADDFKGRFAFSLETSASTAYLQINNLKNEDMATYFCARWGGYDGDYYAMDYWGQGTSVTVSS

SEQ ID NO: 154 - protein, 123 aa, synthetic construct
  REGION 1..123: note = VH-19
EVQLVESGGDLVKPGGSLKLSCAASGFTFSNYGLSWIRQTPDKRLEWVATISSGGSYTYYPDSVKGRFTISRDSAKNTLYLQMSSLKSEDTAMYYCARHAHYYGVSPYYFDYWGQGTCLTVSS

SEQ ID NO: 155 - protein, 119 aa, synthetic construct
  REGION 1..119: note = VH-20
QIQLVQSGPEVKKPGETVKISCKASGYIFTNYGMNWVKQAPGKGLKWMGWINTYTGEPTYTDDFKGRFAFSLETSASTAYLQINNLKNEDTATYFCAKFGNYVGAMDYWGQGTSVTVSS

SEQ ID NO: 156 - protein, 120 aa, synthetic construct
  REGION 1..120: note = VH-21
EVQLQQSGPELVKPGASVKISCKASGNTFTDHNMHWVKQSHGKSLEWIGYIYPYNGGTGYNQKFKSKATLTVDNSSSTVYMELRSLTSEDSAVYYCARGEYDYLAWFAYWGQGTLVTVSA

SEQ ID NO: 157 - protein, 119 aa, synthetic construct
  REGION 1..119: note = VH-22
QIQLVQSGPELKKPGETVKISCKASGYTFTNYGMNWVKQAPGKGLKWMGWINTYTGEPTYVDDFKGRFAFSLETSASTAYLRINNLKNEDTATYFCAKFGNYVGAMDYWGQGTSVTVSS

SEQ ID NO: 158 - protein, 117 aa, synthetic construct
  REGION 1..117: note = VH-23
EVQLVETGGGLVKPGGSLKLSCAASGFTFSSYAMSWVRQTPEKRLEWVASISSGGSTYYPDSVKGRFTISRDNARNILYLQMSSLRSEDTAMYYCTRFTTVVEMDYWGQGTSVTVSS

SEQ ID NO: 159 - protein, 112 aa, synthetic construct
  REGION 1..112: note = VH-24
QVQLQQTGAELVKPGASVKLSCKASGYTFTSYWINWVKQRPGQGLEWIGNIYPGSSSIYYSEKFKSKATLTVDTSSSTAYMQLSSLTSDDSAVYYCARWGYWGQGTTLTVSS

SEQ ID NO: 160 - protein, 121 aa, synthetic construct
  REGION 1..121: note = VH-25
QVTLKESGPGILQPSQTLSLTCSFSGFSLSTFGMGVGWIRQPSGKGLEWLAHIWWDDDKHYNPALKSRLTISKDTSKNQVFLRIANVDTADNATYYCARIDYGDYVGFAYWGQGTLVTVSA

SEQ ID NO: 161 - protein, 112 aa, synthetic construct
  REGION 1..112: note = VL-1
DIVLTQSPLSLPVTPGEPASISCRSSKSLLYKDGKTYLNWFLQKPGQSPQLLIYLMSTHASGVPDRFSGSGSGTDFTLKISRVEAEDVGVYYCQQLVDYPLTFGGGTKLEIK

SEQ ID NO: 162 - protein, 112 aa, synthetic construct
  REGION 1..112: note = VL-2
DIVMTQTPLSLSVTPGQPASISCKSSKSLLHSDGITYLYWYLQKPGQSPQLLIYRMSNLASGVPDRFSGSGSGTDFTLKISRVEAEDVGVYYCAQMLEFPLTFGQGTKLEIK

SEQ ID NO: 163 - protein, 107 aa, synthetic construct
  REGION 1..107: note = VL-3
DILMTQSPSSMSVSLGDTVSITCHASQGINSNMGWLQQKPGKSFKGLIYHATNLEDGVPSRFSGSGSGADYSLTISSLESEDFADYYCVQYAQFPPTFGSGTKLEIK

SEQ ID NO: 164 - protein, 112 aa, synthetic construct
  REGION 1..112: note = VL-4
DVVMTQTPLTLSVAIGQPASISCKSSQSLLYTNGKTYLNWLLQRPGQSPKRLIYLLSKLDSGVPDRFSASGSGTDFTLKISRVEAEDLGVYYCLQSTHFPLTFGAGTKLEMK

SEQ ID NO: 165 - protein, 112 aa, synthetic construct
  REGION 1..112: note = VL-5
DVVMTQTPLTLSVTIGQPASISCKSSQSLFDSDGKTYLNWLLQRPGQSPKRLIYLVSKLDSGVPDRFTGSGSGTDFTLKISRVEAEDLGVYYCWQGTHFPLTFGAGTKLEMK

SEQ ID NO: 166 - protein, 112 aa, synthetic construct
  REGION 1..112: note = VL-6
DVLMTQTPLSLPVSLGDQASISCRSSQSIVHNNGNTYLEWYLQKPGQSPKLLIYKVSNRFSGVPDRFSGSGSGTDFTLKISRVEAEDLGVYYCFQGSHVPLTFGGGTKLELK

SEQ ID NO: 167 - protein, 107 aa, synthetic construct
  REGION 1..107: note = VL-7
DIQMTQTTSSLSASLGDRVTISCSASQGINNYLNWYQQKPDGTVKLLIYYASSLHSGVPSRFSGSGSGTDYSLTISNLEPEDIATYYCQQYSQVPYTFGSGTKLEIK

SEQ ID NO: 168 - protein, 107 aa, synthetic construct
  REGION 1..107: note = VL-8
DIQMNQSPSSLSASLGDTITITCRASQNIYIWLSWYQQKPGNIPKLLIYKASNLHTGVPSRFSGSGSGTDFTLTISTLQPEDIATYFCLQGQSYPLTFGAGTKLEMK

SEQ ID NO: 169 - protein, 107 aa, synthetic construct
  REGION 1..107: note = VL-9
DIQMTQTTSSLSASLGDRVTINCSASQDISNYLNWYQQKPDGTVKLLIYYTSSLLSGVPSRFSGSGSGTDYSLTISNLEPEDIATYFCQQYSKLPYTFGSGTHLEIK

SEQ ID NO: 170 - protein, 112 aa, synthetic construct
  REGION 1..112: note = VL-10
DIVMTQAAFSNPVTLGTSASISCRSSKSLLHSDGITYLYWYLQRPGQSPQLLIYRMSNLASGVPDRFSGSGSGTDFTLRISRVEAEDVGVYYCAQMIEFPLTFGAGTILELK

SEQ ID NO: 171 - protein, 112 aa, synthetic construct
  REGION 1..112: note = VL-11
DVVMTQTPLTLSVAIGQPASISCKSSQSLLYTNGKTYLNWLLQRPGQSPKRLIYLVSKLDSGVPDRFSGSGSGTDFTLKISRVEAEDLGVYYCLQSTHFPLTFGAGTKLELK

SEQ ID NO: 172 - protein, 112 aa, synthetic construct
  REGION 1..112: note = VL-12
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 173 - protein, 112 aa, synthetic construct
  REGION 1..112: note = VL-13
NIWLTQSPSSLAVSAGEMVTMTCKSSQSVLYSSNQKNYLAWYQQKPGQSPKLLIYWASTRESGVPDRFTGSGSGTDFSLTISSVQAEDLAVYYCHQYLSSLTFGAGTKLELK

SEQ ID NO: 174 - protein, 112 aa, synthetic construct
  REGION 1..112: note = VL-14
NIMMTQSPSSLAVSAGEKVTMSCKSSQSVLYSSNQKNYLAWYQQKPGQSPKLLIYWASTRESGVPDRFTGSGSGTDFTLTISSVQPEDLAVYYCHQYLSSLTFGAGTKLELK

SEQ ID NO: 175 - protein, 112 aa, synthetic construct
  REGION 1..112: note = VL-15
NIMMTQSPSSLAVSAGQKVTMSCKSSQSVLYSSSQKNYLAWYQQKPGQSPKLLIYWASTRESGVPDRFSGSGSGTDFTLTISSVQPEDLAVYYCHQYLSSLTFGAGTKLELK

SEQ ID NO: 176 - protein, 112 aa, synthetic construct
  REGION 1..112: note = VL-16
DVVMTQTPLSLPVSLGDQASISCRSSQSLVHNSGNTYLHWYLQRPGQSPNLLIYKVSNRFSGVPDRFSGSGSGTDFTLKISRVAAEDLGVYFCSQSTHVPPTFGGGTKLEIK

SEQ ID NO: 177 - protein, 109 aa, synthetic construct
  REGION 1..109: note = VL-17
QAVVTQESALTTSPGETVTLTCRSSTGAVTTSNYANWVQEKPDHLFTGLIGGINNRVPGVPARFSGSLIGDKAALTITGAQTEDEAIYFCALWYSNHWVFGGGTKLTVL

SEQ ID NO: 178 - protein, 112 aa, synthetic construct
  REGION 1..112: note = VL-18
DIVMTQAAFSNPVTLGTSASISCRSSKSLLHTNGITFLYWYLQKPGQSPQPLIYQMSNLASGVPDRFSSSGSGTDFTLRISRVEAEDVGVYYCAQNLELPWTFGGGTKLEIN

SEQ ID NO: 179 - protein, 112 aa, synthetic construct
  REGION 1..112: note = VL-19
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELK

SEQ ID NO: 180 - protein, 113 aa, synthetic construct
  REGION 1..113: note = VL-20
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPYTFGGGAKLEIK

SEQ ID NO: 181 - protein, 107 aa, synthetic construct
  REGION 1..107: note = VL-21
DIRMTQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLELK

SEQ ID NO: 182 - protein, 107 aa, synthetic construct
  REGION 1..107: note = VL-22
EIVLTQSPASLSVTTGEKVTIRCITSIDIDDDMNWYQQKPGEPPKLLISEGNTLRPGVPSRFSSSGYGTDFVFTIENTLSEDVADYYCLQSDNKPLTFGAGTKLELK

SEQ ID NO: 183 - protein, 107 aa, synthetic construct
  REGION 1..107: note = VL-23
DIQVIQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLELK

SEQ ID NO: 184 - protein, 107 aa, synthetic construct
  REGION 1..107: note = VL-24
EIKMNQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLELK

SEQ ID NO: 185 - protein, 109 aa, synthetic construct
  REGION 1..109: note = VL-25
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVL

SEQ ID NO: 186 - protein, 112 aa, synthetic construct
  REGION 1..112: note = VL-26
DVLMTQTPLSLPVSLGDQASISCRSSQDIVHSSGNTYLEWYLQKPGQSLKLLIYKVSNRFSGVPDRFSASGSGTDFTLKISRVEAEDLGVYYCFQGSHVPPTFGSGTKLEIK

SEQ ID NO: 187 - protein, 112 aa, synthetic construct
  REGION 1..112: note = VL-27
DIGMRQAAFSNPVTLGTSASISCRSSKSLLYSNGITYLYWFLQKPGQSPQLLIYQMSNLASGVPDRFSSSGSGTDFTLRISRVEADDVGVYYCAQNLELPWTFGGGTKLEIK

SEQ ID NO: 188 - protein, 461 aa, synthetic construct
  REGION 1..461: note = HC-1
MGSTAILGLLLAVLQGVCAQVQLVQSGSELKKPGASVKVSCKASGYTFTNYGMNWVRQAPGQGLKWMGWINTNTGEPTYVEEFTGRFVFSLETSVSTAYLQISSLKAEDTAVYFCAPYDNFFAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 189 - protein, 466 aa, synthetic construct
  REGION 1..466: note = HC-2
MDPKGSLSWRILLFLSLAFELSYGQVQLVQSGAEVKKPGASVKVSCKASGYSFTNYYMHWVRQAPGQRLEWMGWIYPGSGNTNYNEKFQGRVTITADTSASTAYMELSSLRSEDTAVYYCSTAPGGFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 190 - protein, 476 aa, synthetic construct
  REGION 1..476: note = HC-3
MDPKGSLSWRILLFLSLAFELSYGQVQLKQSGAELVRPGASVKLSCKASGYTFTDYYINWVKQRPGQGLEWIARIYPGTGNTDYNEKFKGRATLTAEKSSSTAYMQLSSLTSEDSAVYFCARFAYYYGSGGYFDYWGHGTTLTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 191 - protein, 473 aa, synthetic construct
  REGION 1..473: note = HC-4
MDPKGSLSWRILLFLSLAFELSYGQIQLVQSGPELKKPGETVKISCKTSGYKFKTYVMSWVKQAPGKALKWMGWINTYSGVPTYADDFKGRFAFSLETSASTAYLEIINLKNEDTATYFCARDGNYGDPMDYWGQGTSVTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 192 - protein, 471 aa, synthetic construct
  REGION 1..471: note = HC-5
MDPKGSLSWRILLFLSLAFELSYGQIQLVQSGPELKKPGETVKISCKASGYTFTTYGMGWVKQAPGKDLKWMGWINTYSGVPTYADDFKGRFAFSLETSASTAYLQISNLKNEDTATYFCSTPYEYDGAYWGQGTLVTVSAAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 193 - protein, 467 aa, synthetic construct
  REGION 1..467: note = HC-6
MDPKGSLSWRILLFLSLAFELSYGEVQLVESGGGLVKPGGSLKLSCAASGFAFSSYAMSWVRQTPEKRLEWVATISDGGVYTYYTDHVKGRFTISRDNAEDNLYLQMSHLKSEDTAMYYCVRDGGYWGQGTTLTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 194 - protein, 470 aa, synthetic construct
  REGION 1..470: note = HC-7
MDPKGSLSWRILLFLSLAFELSYGQIQLVQSGPELKKPGETVKISCKASGYTFTTYAMSWVKQAPGKGLKWMGWINTYSGVPTYADDLKGRFAFSLETSASTAYLQINNLKNEDTATYFCARGPYAMDYWGQGTSVTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 195 - protein, 469 aa, synthetic construct
  REGION 1..469: note = HC-8
MDPKGSLSWRILLFLSLAFELSYGEVQLQQSGPELVKPGASVKISCKASGYTFTDFYINWVKQSHGKSLEWIGDINPKNGGINYNPKFKIKATLTVDKSSSTSYMDLRGLTSEDSAVYYCTSGYGFPYWGQGTLVTVSAAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 196 - protein, 470 aa, synthetic construct
  REGION 1..470: note = HC-9
MDPKGSLSWRILLFLSLAFELSYGQIQLVQSGPELKKPGESVKISCKASGYAFTTYGMSWVQQAPGKGLKWMGWVNTYSGVPTCADDFKGRFAFSLETSASTAYLQINNLRNEDTATYFCARGPYAMDYWGQGTSVTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 197 - protein, 470 aa, synthetic construct; identical to SEQ ID NO: 194
  REGION 1..470: note = HC-10

SEQ ID NO: 198 - protein, 474 aa, synthetic construct
  REGION 1..474: note = HC-11
MDPKGSLSWRILLFLSLAFELSYGQVQLQQSGPELVKPGASVKISCKASGYTFTDYYINWVKQRPGQGLEWIGWIYPGSNDTKYNEKFKGKATLTVDTSSSTAYMQLGSLTSEDSAVYFCANYFGCSGWFFDVWGTGTTVTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 199 - protein, 473 aa, synthetic construct; identical to SEQ ID NO: 191
  REGION 1..473: note = HC-12

SEQ ID NO: 200 - protein, 467 aa, synthetic construct
  REGION 1..467: note = HC-13
MDPKGSLSWRILLFLSLAFELSYGEVQLVESGGGLVKPGGSLKLSCAASGFTFSSYAMSWVRQTPEKRLEWVATISDGGIYTYYPDNVKGRFTISRDNAKNNLFLQMSHLKSEDTAMYYCVRDGGYWGQGTTLTVSSAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 201 - protein, 448 aa, synthetic construct
  REGION 1..448: note = HC-14
QIQLVQSGPELKKPGETVKISCKASGYTFTNYGMNWVKQAPGKGLKWMGWINTYTGEPSYADDFKGRFAFSLETSASTAYLQINNLKNEDMATYFCARWGGYAGDYYAMDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 202 - protein, 448 aa, synthetic construct
  REGION 1..448: note = HC-15
QIQLVQSGPELKKPGETVKISCKASGYTFTNYGMNWVKQAPGKGLKWMGWINTYTGEPSYADDFKGRFAFSLETSASTAYLQINNLKNEDMATYFCARWGGYDGDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 203 - protein, 448 aa, synthetic construct
  REGION 1..448: note = HC-16
QIQLVQSGPELKKPGETVKISCKASGYTFTNFGMNWVKQAPGKGLKWMGWINTYTGEPSYADDFKGRFAFSLETSASTAYLQINNLKNEDIATYFCARWGGYAGDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 204 - protein, 442 aa, synthetic construct
  REGION 1..442: note = HC-17
EVQLLESGGGLVKPGGSLKLSCAASGFTFSSYAMSWVRQTPEKRLEWVASISSGGDTYYPDSVKGRFTISRDNARNILYLQMSSLRSEDTAMYYCASYGNSLFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 205 - protein, 448 aa, synthetic construct; identical to SEQ ID NO: 202
  REGION 1..448: note = HC-18

SEQ ID NO: 206 - protein, 440 aa, synthetic construct
  REGION 1..440: note = HC-19
QIQLVQSGPELKKPGETVKISCKASGYTFTNYGMNWVKQAPGKGLKWMGWINTYTGEPTYAGDLKGRFAFSLETSASTAYLQINNLKNEDTATYFCVRYTMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 207 - protein, 450 aa, synthetic construct
  REGION 1..450: note = HC-20
QVTLKESGPGILQPSQTLSLTCSFSGFSLSTSGMGVGWIRQPSGKGLEWLAHIWWDDDKYYNTALKSGLTISKDASKNQVFLKIASMDTADTATYYCARNLYDGSYGYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 208 - protein, 448 aa, synthetic construct
  REGION 1..448: note = HC-21
QIQLVQSGPELKKPGETVKISCKASGYTFTNYGMNWVKQAPGKGLKWMGWINTYTGEPAYADDFKGRFAFSLETSASTAYLQINNLKNEDMATYFCARWGGYDGDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 209 - protein, 449 aa, synthetic construct
  REGION 1..449: note = HC-22
EVQLVESGGDLVKPGGSLKLSCAASGFTFSNYGLSWIRQTPDKRLEWVATISSGGSYTYYPDSVKGRFTISRDSAKNTLYLQMSSLKSEDTAMYYCARHAHYYGVSPYYFDYWGQGTCLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 210 - protein, 445 aa, synthetic construct
  REGION 1..445: note = HC-23
QIQLVQSGPEVKKPGETVKISCKASGYIFTNYGMNWVKQAPGKGLKWMGWINTYTGEPTYTDDFKGRFAFSLETSASTAYLQINNLKNEDTATYFCAKFGNYVGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 211 - protein, 446 aa, synthetic construct
  REGION 1..446: note = HC-24
EVQLQQSGPELVKPGASVKISCKASGNTFTDHNMHWVKQSHGKSLEWIGYIYPYNGGTGYNQKFKSKATLTVDNSSSTVYMELRSLTSEDSAVYYCARGEYDYLAWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 212 - protein, 445 aa, synthetic construct
  REGION 1..445: note = HC-25
QIQLVQSGPELKKPGETVKISCKASGYTFTNYGMNWVKQAPGKGLKWMGWINTYTGEPTYVDDFKGRFAFSLETSASTAYLRINNLKNEDTATYFCAKFGNYVGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 213 - protein, 445 aa, synthetic construct; identical to SEQ ID NO: 210
  REGION 1..445: note = HC-26

SEQ ID NO: 214 - protein, 443 aa, synthetic construct
  REGION 1..443: note = HC-27
EVQLVETGGGLVKPGGSLKLSCAASGFTFSSYAMSWVRQTPEKRLEWVASISSGGSTYYPDSVKGRFTISRDNARNILYLQMSSLRSEDTAMYYCTRFTTVVEMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 215 - protein, 438 aa, synthetic construct
  REGION 1..438: note = HC-28
QVQLQQTGAELVKPGASVKLSCKASGYTFTSYWINWVKQRPGQGLEWIGNIYPGSSSIYYSEKFKSKATLTVDTSSSTAYMQLSSLTSDDSAVYYCARWGYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 216 - protein, 447 aa, synthetic construct
  REGION 1..447: note = HC-29
QVTLKESGPGILQPSQTLSLTCSFSGFSLSTFGMGVGWIRQPSGKGLEWLAHIWWDDDKHYNPALKSRLTISKDTSKNQVFLRIANVDTADNATYYCARIDYGDYVGFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 217 - protein, 241 aa, synthetic construct
  REGION 1..241: note = LC-1
MDMRVPAQLLGLLLLWLRGARCDIVLTQSPLSLPVTPGEPASISCRSSKSLLYKDGKTYLNWFLQKPGQSPQLLIYLMSTHASGVPDRFSGSGSGTDFTLKISRVEAEDVGVYYCQQLVDYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 239 aa, synthetic construct
  REGION 1..239: note = LC-2
METDTLLLWVLLLWVPGSTGDIVMTQTPLSLSVTPGQPASISCKSSKSLLHSDGITYLYWYLQKPGQSPQLLIYRMSNLASGVPDRFSGSGSGTDFTLKISRVEAEDVGVYYCAQMLEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 234 aa, synthetic construct
  REGION 1..234: note = LC-3
METDTLLLWVLLLWVPGSTGDILMTQSPSSMSVSLGDTVSITCHASQGINSNMGWLQQKPGKSFKGLIYHATNLEDGVPSRFSGSGSGADYSLTISSLESEDFADYYCVQYAQFPPTFGSGTKLEIKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 220 - protein, 239 aa, synthetic construct
  REGION 1..239: note = LC-4
METDTLLLWVLLLWVPGSTGDVVMTQTPLTLSVAIGQPASISCKSSQSLLYTNGKTYLNWLLQRPGQSPKRLIYLLSKLDSGVPDRFSASGSGTDFTLKISRVEAEDLGVYYCLQSTHFPLTFGAGTKLEMKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 221 - protein, 239 aa, synthetic construct
  REGION 1..239: note = LC-5
METDTLLLWVLLLWVPGSTGDVVMTQTPLTLSVTIGQPASISCKSSQSLFDSDGKTYLNWLLQRPGQSPKRLIYLVSKLDSGVPDRFTGSGSGTDFTLKISRVEAEDLGVYYCWQGTHFPLTFGAGTKLEMKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 222 - protein, 239 aa, synthetic construct
  REGION 1..239: note = LC-6
METDTLLLWVLLLWVPGSTGDVLMTQTPLSLPVSLGDQASISCRSSQSIVHNNGNTYLEWYLQKPGQSPKLLIYKVSNRFSGVPDRFSGSGSGTDFTLKISRVEAEDLGVYYCFQGSHVPLTFGGGTKLELKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 223 - protein, 234 aa, synthetic construct
  REGION 1..234: note = LC-7
METDTLLLWVLLLWVPGSTGDIQMTQTTSSLSASLGDRVTISCSASQGINNYLNWYQQKPDGTVKLLIYYASSLHSGVPSRFSGSGSGTDYSLTISNLEPEDIATYYCQQYSQVPYTFGSGTKLEIKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 224 - protein, 234 aa, synthetic construct
  REGION 1..234: note = LC-8
METDTLLLWVLLLWVPGSTGDIQMNQSPSSLSASLGDTITITCRASQNIYIWLSWYQQKPGNIPKLLIYKASNLHTGVPSRFSGSGSGTDFTLTISTLQPEDIATYFCLQGQSYPLTFGAGTKLEMKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 225 - protein, 234 aa, synthetic construct
  REGION 1..234: note = LC-9
METDTLLLWVLLLWVPGSTGDIQMTQTTSSLSASLGDRVTINCSASQDISNYLNWYQQKPDGTVKLLIYYTSSLLSGVPSRFSGSGSGTDYSLTISNLEPEDIATYFCQQYSKLPYTFGSGTHLEIKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 226 - protein, 239 aa, synthetic construct
  REGION 1..239: note = LC-10
METDTLLLWVLLLWVPGSTGDIVMTQAAFSNPVTLGTSASISCRSSKSLLHSDGITYLYWYLQRPGQSPQLLIYRMSNLASGVPDRFSGSGSGTDFTLRISRVEAEDVGVYYCAQMIEFPLTFGAGTILELKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 227 - protein, 239 aa, synthetic construct
  REGION 1..239: note = LC-11
METDTLLLWVLLLWVPGSTGDVVMTQTPLTLSVAIGQPASISCKSSQSLLYTNGKTYLNWLLQRPGQSPKRLIYLVSKLDSGVPDRFSGSGSGTDFTLKISRVEAEDLGVYYCLQSTHFPLTFGAGTKLELKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 228 - protein, 239 aa, synthetic construct
  REGION 1..239: note = LC-12
METDTLLLWVLLLWVPGSTGDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 229 - protein, 219 aa, synthetic construct
  REGION 1..219: note = LC-13
NIWLTQSPSSLAVSAGEMVTMTCKSSQSVLYSSNQKNYLAWYQQKPGQSPKLLIYWASTRESGVPDRFTGSGSGTDFSLTISSVQAEDLAVYYCHQYLSSLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 219 aa, synthetic construct
  REGION 1..219: note = LC-14
NIMMTQSPSSLAVSAGEKVTMSCKSSQSVLYSSNQKNYLAWYQQKPGQSPKLLIYWASTRESGVPDRFTGSGSGTDFTLTISSVQPEDLAVYYCHQYLSSLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 219 aa, synthetic construct
  REGION 1..219: note = LC-15
NIMMTQSPSSLAVSAGQKVTMSCKSSQSVLYSSSQKNYLAWYQQKPGQSPKLLIYWASTRESGVPDRFSGSGSGTDFTLTISSVQPEDLAVYYCHQYLSSLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 219 aa, synthetic construct
  REGION 1..219: note = LC-16
DVVMTQTPLSLPVSLGDQASISCRSSQSLVHNSGNTYLHWYLQRPGQSPNLLIYKVSNRFSGVPDRFSGSGSGTDFTLKISRVAAEDLGVYFCSQSTHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 233 - protein, 216 aa, synthetic construct
  REGION 1..216: note = LC-17
QAVVTQESALTTSPGETVTLTCRSSTGAVTTSNYANWVQEKPDHLFTGLIGGINNRVPGVPARFSGSLIGDKAALTITGAQTEDEAIYFCALWYSNHWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - protein, 219 aa, synthetic construct
  REGION 1..219: note = LC-18
DIVMTQAAFSNPVTLGTSASISCRSSKSLLHTNGITFLYWYLQKPGQSPQPLIYQMSNLASGVPDRFSSSGSGTDFTLRISRVEAEDVGVYYCAQNLELPWTFGGGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 219 aa, synthetic construct
  REGION 1..219: note = LC-19
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 236 - protein, 220 aa, synthetic construct
  REGION 1..220: note = LC-20
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPYTFGGGAKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 214 aa, synthetic construct
  REGION 1..214: note = LC-21
DIRMTQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 214 aa, synthetic construct
  REGION 1..214: note = LC-22
EIVLTQSPASLSVTTGEKVTIRCITSIDIDDDMNWYQQKPGEPPKLLISEGNTLRPGVPSRFSSSGYGTDFVFTIENTLSEDVADYYCLQSDNKPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 214 aa, synthetic construct
  REGION 1..214: note = LC-23
DIQVIQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 214 aa, synthetic construct
  REGION 1..214: note = LC-24
EIKMNQSPSSLSASLGDTITITCHASQNINVWLSWYQQKPGNIPKLLIYKASNLHTGVPSRFSGSGSGTGFTLTISSLQPEDIATYYCQQGQSYPYTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 216 aa, synthetic construct
  REGION 1..216: note = LC-25
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 219 aa, synthetic construct
  REGION 1..219: note = LC-26
DVLMTQTPLSLPVSLGDQASISCRSSQDIVHSSGNTYLEWYLQKPGQSLKLLIYKVSNRFSGVPDRFSASGSGTDFTLKISRVEAEDLGVYYCFQGSHVPPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 219 aa, synthetic construct
  REGION 1..219: note = LC-27
DIGMRQAAFSNPVTLGTSASISCRSSKSLLYSNGITYLYWFLQKPGQSPQLLIYQMSNLASGVPDRFSSSGSGTDFTLRISRVEADDVGVYYCAQNLELPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 42 aa, Homo sapiens [sequence omitted]

SEQ ID NO: 245 - protein, 8 aa, synthetic construct
  REGION 1..8: note = VH_CDR1_19
GYTFTSYD

SEQ ID NO: 246 - protein, 8 aa, synthetic construct
  REGION 1..8: note = VH_CDR1_20
GFTFSNYW

SEQ ID NO: 247 - protein, 8 aa, synthetic construct
  REGION 1..8: note = VH_CDR2_17
IYPGDGST

SEQ ID NO: 248 - protein, 10 aa, synthetic construct
  REGION 1..10: note = VH_CDR2_18
IRLKSNNYAT

SEQ ID NO: 249 - protein, 11 aa, synthetic construct
  REGION 1..11: note = VH_CDR3_22
AREAASNAMDY

SEQ ID NO: 250 - protein, 13 aa, synthetic construct
  REGION 1..13: note = VH_CDR3_23
TRHYGFPYWYFDV

SEQ ID NO: 251 - protein, 10 aa, synthetic construct
  REGION 1..10: note = VL_CDR1_21
KSVSTSGYSY

SEQ ID NO: 252 - protein, 10 aa, synthetic construct
  REGION 1..10: note = VL_CDR1_22
ESVDSYGNSF

SEQ ID NO: 253 - skipped

SEQ ID NO: 254 - protein, 9 aa, synthetic construct
  REGION 1..9: note = VL_CDR3_20
QHSRELPLT

SEQ ID NO: 255 - protein, 9 aa, synthetic construct
  REGION 1..9: note = VL_CDR3_21
QQNNEDPWT

SEQ ID NO: 256 - protein, 118 aa, synthetic construct
  REGION 1..118: note = VH-26
QVQLQQSGPELVRPGALVKISCKASGYTFTSYDINWVKQRPGQGLEWIGWIYPGDGSTKYNEKFKGKATLTADKSSSTAYMQLSSLTSENSAVYFCAREAASNAMDYWGQGTSVTVSS

SEQ ID NO: 257 - protein, 122 aa, synthetic construct
  REGION 1..122: note = VH-27
EVKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTRHYGFPYWYFDVWGAGTTVTVSS

SEQ ID NO: 258 - protein, 111 aa, synthetic construct
  REGION 1..111: note = VL-28
DIVLTQSPASLGVSLGQRATISCRASKSVSTSGYSYMHWYQQKPGQPPKLLIYLASILESGVPARFSGSGSGTDFTLNIHPVEEEDAAIYYCQHSRELPLTFGAGTKLELK

SEQ ID NO: 259 - protein, 111 aa, synthetic construct
  REGION 1..111: note = VL-29
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIK

SEQ ID NO: 260 - DNA, 348 bp, synthetic construct
  misc_feature 1..348: note = TB001 (IMT001-4) VH nucleic acid
  source 1..348: mol_type = other DNA
caggtgcagctggtgcagtccggctccgagctgaaaaaacccggcgcctccgtgaaagtgtcctgcaaagcctccggctacacattcacaaactacggcatgaactgggtgaggcaggcccccggccagggcctgaaatggatgggctggatcaacacaaacacaggcgagcccacatacgtggaggagttcacaggcaggttcgtgttctccctggagacatccgtgtccacagcctacctgcagatctcctccctgaaagccgaggacacagccgtgtacttctgcgccccctacgacaacttcttcgcctactggggccagggcacaacagtgacagtgtcctcc

SEQ ID NO: 261 - DNA, 357 bp, synthetic construct
  misc_feature 1..357: note = TB006(4A11.H3L1) VH nucleic acid
  source 1..357: mol_type = other DNA
cagattcaattggttcagagcggctcagaactgaaaaagcctggagccagcgttaaagtgagctgtaaagccagtggatacacattcaccacttacgtcatgtcctgggttagacaggcccctggacaggggctggaatggatgggctggataaacactcactctggagtgcccacatacgccgacgactttacaggcaggttcgtgttttccctagatactagtgtgaatactgcatacttgcaaatctcttctctgaaagcagaggataccgccgtgtacttttgcacccgcgatgggaatgatggggatgccatggacaactggggccaggggacgactgtgacagtgtcttca

SEQ ID NO: 262 - DNA, 366 bp, synthetic construct
  misc_feature 1..366: note = 12G5.D7 VH nucleic acid
  source 1..366: mol_type = other DNA
caggtgcagctgaagcagtctggggctgagctggtgaggcctggggcttcagtgaagctgtcctgcaaggcttctggctacactttcactgactactatataaactgggtgaagcagaggcctggacagggacttgagtggattgcaaggatttatcctggaactggtaatactgactacaatgagaagttcaagggcagggccacactgactgcagaaaactcctccagcactgcctacatgcagctcagcagtctgacatctgaggactctgctgtctatttctgtgcaagatttgcgtattactacggtagtggagggtactttgactactggggccacggcaccactctcacagtctcctca

SEQ ID NO: 263 - DNA, 357 bp, synthetic construct
  misc_feature 1..357: note = 13A12.2E5 VH nucleic acid
  source 1..357: mol_type = other DNA
cagatccagttggtacagtctggacctgagctgaagaagcctggagagacagtcaagatctcctgcaagacttctgggtataaattcaaaacctatgtgatgagctgggtgaaacaggctccaggaaaggctttaaagtggatgggctggataaacacctactctggagtgccaacatatgctgatgacttcaagggacgatttgccttctctttggaaacctctgccagcactgcctatttggagatcatcaacctcaaaaatgaggacacggctacatatttctgtgcaagagatggtaactacggggatcctatggactactggggtcaaggaacctcagtcaccgtctcctca

SEQ ID NO: 264 - DNA, 351 bp, synthetic construct
  misc_feature 1..351: note = 14H10.2C9 (14H10) VH nucleic acid
  source 1..351: mol_type = other DNA
cagatccagttggtacagtctggacctgagctgaagaagcctggagagacagtcaagatctcctgcaaggcttctgggtataccttcacaacctatggaatgggctgggtgaaacaggctccaggaaaggatttaaagtggatgggctggataaacacctactctggagtgccaacatatgctgatgacttcaagggacggtttgccttctctttggaaacctctgccagcactgcctatttgcagatcagcaacctcaaaaatgaagacacggctacatatttctgttcaaccccttatgaatacgacggggcttactggggccaggggactctggtcactgtctctgca

SEQ ID NO: 265 - DNA, 339 bp, synthetic construct
  misc_feature 1..339: note = 15F10.2D6 (15F10) VH nucleic acid
  source 1..339: mol_type = other DNA
gaagtacagctggtggagtctgggggaggcttagtgaagcctggagggtccctgaaactctcctgtgcagcctctggattcgctttcagttcctatgccatgtcttgggttcgccagactccggaaaagaggctggagtgggtcgcaaccattagtgatggtggtgtttacacctactatacagaccatgtaaagggccgattcaccatctccagagacaatgccgaggacaacctgtacctgcaaatgagccatctgaagtctgaggacacagccatgtattattgtgtgagagatgggggctactggggccaaggcaccactctcacagtctcctca

SEQ ID NO: 266 - DNA, 348 bp, synthetic construct
  misc_feature 1..348: note = 19B5.2E6 (19B5) VH nucleic acid
  source 1..348: mol_type = other DNA
cagatccaacttgttcagagcgggcctgaattgaagaaaccaggcgagactgtaaagatctcctgtaaggccagtggctatacattcaccacctacgcaatgagctgggtcaagcaggcacccggaaagggtctgaagtggatgggttggataaatacatactcaggcgtcccaacctacgctgatgatttgaaggggcgctttgccttttctcttgaaacctcagcatcaaccgcctacttgcagataaataacctcaaaaatgaagacaccgctacctatttctgcgcaagggggccttatgctatggactactggggccagggcacctctgtgactgtaagttct

SEQ ID NO: 267 - DNA, 345 bp, synthetic construct
  misc_feature 1..345: note = 20D11.2C6 (20D11) VH nucleic acid
  source 1..345: mol_type = other DNA
gaggtccagctgcaacaatctggacctgaactggtgaagcctggggcttcagtgaagatatcctgtaaggcttctggatacacgttcactgacttctacataaactgggtgaagcagagccatggaaagagccttgagtggattggagatattaatcctaagaatggtggtattaactacaacccgaagttcaagatcaaggccacattgactgtagacaagtcctccagcacatcctacatggacctccgcggcctgacatctgaggactctgcagtctattactgcacctcaggctacggatttccttactggggccaagggactctggtcactgtctctgca

SEQ ID NO: 268 - DNA, 348 bp, synthetic construct
  misc_feature 1..348: note = 20H5.A3 (20H5) VH nucleic acid
  source 1..348: mol_type = other DNA
cagatccagttggtacagtctggacctgagctgaagaagcctggagagtcagtcaagatctcctgcaaggcttctggttatgccttcacaacctatggaatgagctgggtgcaacaggctccaggaaagggtttaaagtggatgggctgggtaaacacctactctggagtgccaacatgtgctgatgacttcaagggacggtttgccttctctttggaaacctctgccagcactgcctatttgcagatcaacaacctcagaaatgaggacacggctacatatttctgtgcaagagggccctatgctatggactactggggtcaaggaacctcagtcaccgtctcctca

SEQ ID NO: 269 - DNA, 348 bp, synthetic construct
  misc_feature 1..348: note = 23H9.2E4 (23H9) VH nucleic acid
  source 1..348: mol_type = other DNA
cagatccagttggtacagtctggacctgagctgaagaagcctggagagacagtcaagatctcctgcaaggcttctgggtataccttcacaacctatgcaatgagctgggtgaaacaggctccaggaaagggtttaaagtggatgggctggataaacacctactctggagtgccaacatatgctgatgacctcaagggacggtttgccttctctttggaaacctctgccagcactgcctatttgcagatcaacaacctcaaaaatgaggacacggctacatatttctgtgcaagagggccctatgctatggactactggggtcaaggaacctcagtcaccgtctcctca

SEQ ID NO: 270 - DNA, 360 bp, synthetic construct
  misc_feature 1..360: note = 2D10.2B2 (2D10) VH nucleic acid
  source 1..360: mol_type = other DNA
caagtacagctccagcaatcaggacctgaacttgttaagccaggtgcaagcgtcaaaataagttgtaaagcatcaggttacacctttacagactactatatcaactgggtcaagcaacggcctggtcagggacttgaatggataggttggatctaccctgggtcacaagacaccaagtacaatgaaaaatttaaggggaaagccactctgacagtagatactagctcaagtaccgcctacatgcagctcggcagcctcacttccgaggactctgccgtatatttctgtgccaactacttcgggtcttctgggtggttctttgatgtgtggggaaccggcaccaccgtaaccgtttcctca

SEQ ID NO: 271 - DNA, 357 bp, synthetic construct
  misc_feature 1..357: note = 3B11.2G2 (3B11) VH nucleic acid
  source 1..357: mol_type = other DNA
cagatccagttggtacagtctggacctgagctgaagaagcctggagagacagtcaagatctcctgcaagacttctgggtataaattcaaaacctatgtaatgagctgggtgaaacaggctccaggaaaggctttaaagtggatgggctggataaacacctactctggagtgccaacatatgctgatgacttcaagggacggtttgccttctctttggaaacctctgccagcactgcctatttggagatcatcaacctcaaaaatgaggacacggctacatatttctgtgcaagagatggtaattacggggatcctatggactactggggtcaaggaacctcagtcaccgtctcctca

SEQ ID NO: 272 - DNA, 339 bp, synthetic construct
  misc_feature 1..339: note = 7D8.2D8 (7D8) VH nucleic acid
  source 1..339: mol_type = other DNA
gaagtgcagctggtggagtctgggggaggcttagtgaagcctggagggtccctgaaactctcctgtgcagcctctggattcactttcagtagctatgccatgtcttgggttcgccagactccggaaaagaggctggagtgggtcgcaaccatcagtgatggtggtatttacacctactatccagacaatgtaaagggccgattcaccatttccagagacaatgccaagaacaacctgttcctgcaaatgagccatctgaagtctgaggatacagccatgtattactgtgtaagagatgggggctactggggccaaggcaccactctcacagtctcctca

SEQ ID NO: 273 - DNA, 366 bp, synthetic construct
  misc_feature 1..366: note = F846C.1B2 VH nucleic acid
  source 1..366: mol_type = other DNA
cagatccagttggtgcagtctggacctgagctgaagaagcctggagagacagtcaagatctcctgcaaggcttctgggtataccttcacaaactatggaatgaattgggtgaagcaggctccaggaaagggtttaaagtggatgggctggataaacacctacactggagagccatcatatgctgatgacttcaagggacggtttgccttctctttggaaacctctgccagcactgcctatttgcagatcaacaacctcaaaaatgaggacatggctacatatttctgtgcaagatgggggggttacgctggggattactatgctatggacttctggggtcaaggaacctcagtcaccgtctcctca

SEQ ID NO: 274 - DNA, 366 bp, synthetic construct
  misc_feature 1..366: note = F846C.1F5 (846.1F5) VH nucleic acid
  source 1..366: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccatcatatgctgatgacttcaagggacggtttgccttctctttggaaacctctgccagcactgcctatttgcagatcaacaacctcaaaaatgaggacatggctacatatttctgtgcaagatgggggggttacgatggggattactatgctatggactactggggtcaaggaacctcagtcaccgtctcctca

SEQ ID NO: 275 - DNA, 366 bp, synthetic construct
  misc_feature 1..366: note = F846C.1H12 (846.1H12) VH nucleic acid
  source 1..366: mol_type = other DNA
cagatccagttggtgcagtctggacctgagctgaagaagcctggagagacagtcaagatctcctgcaaggcttctgggtataccttcacaaactttggaatgaactgggtgaagcaggctccaggaaagggtttaaagtggatgggctggataaacacctacactggagagccatcatatgctgatgacttcaagggacggtttgccttctctttggaaacctctgccagcactgcctatttgcagatcaacaacctcaaaaatgaggacattgctacatatttctgtgcaagatgggggggttacgctggggattactatgctatggactactggggtcaaggaacctcagtcaccgtctcctca

SEQ ID NO: 276 - DNA, 348 bp, synthetic construct
  misc_feature 1..348: note = F846C.1H5 (846.1H5) VH nucleic acid
  source 1..348: mol_type = other DNA
gaggtccagctgttggagtctgggggaggcttagtgaagcctggagggtccctgaaactctcctgtgcagcctctggattcactttcagcagctatgccatgtcttgggttcgccagactccagagaagaggctggagtgggtcgcatccattagtagtggtggtgacacctactatccagacagtgtgaagggccgattcaccatctctagagataatgccaggaacatcctgtacctgcaaatgagcagtctgaggtctgaggacacggccatgtattactgtgcaagttatggtaactccctctttgactactggggccaaggcaccactctcacagtctcctca

SEQ ID NO: 277 - DNA, 366 bp, synthetic construct; identical to SEQ ID NO: 274
  misc_feature 1..366: note = F846C.2H3 (846.2H3) VH nucleic acid
  source 1..366: mol_type = other DNA

SEQ ID NO: 278 - DNA, 342 bp, synthetic construct
  misc_feature 1..342: note = F846TC.14A2 (846T.14A2) VH nucleic acid
  source 1..342: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctggtgacctcaagggacggtttgccttctctttggaaacctctgccagcactgcctatttgcagatcaacaacctcaaaaatgaggacacggctacatatttctgtgtaagatatactatggactactggggtcaaggaacctcagtcaccgtctcctca

SEQ ID NO: 279 - DNA, 372 bp, synthetic construct
  misc_feature 1..372: note = F846TC.14E4 (846T.14E4) VH nucleic acid
  source 1..372: mol_type = other DNA
caggttactctgaaagagtctggccctgggatattgcagccctcccagaccctcagtctgacttgttctttctctgggttttcactgagcacttctggtatgggtgtaggctggattcgtcagccttcagggaagggtctggagtggctggcacacatttggtgggatgatgataagtactataacacagccctgaagagcgggctcacaatctccaaggatgcctccaaaaaccaggtcttcctcaagatcgccagtatggacactgcagatactgccacatactactgtgctcgaaacctctatgatggttcctacgggtactatgctatggactactggggtcaaggaacctcagtcaccgtctcctca

SEQ ID NO: 280 - DNA, 366 bp, synthetic construct
  misc_feature 1..366: note = F846TC.16B5 (846T.16B5) VH nucleic acid
  source 1..366: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccagcatatgctgatgacttcaagggacggtttgccttctctttggaaacctctgccagcactgcctatttgcagatcaacaacctcaaaaatgaggacatggctacatatttctgtgcaagatgggggggttacgatggggattactatgctatggactactggggtcaaggaacctcagtcaccgtctcctca

SEQ ID NO: 281 - DNA, 369 bp, synthetic construct
  misc_feature 1..369: note = F846TC.7F10 (846T.7F10) VH nucleic acid
  source 1..369: mol_type = other DNA
gaagtgcagctggtggagtctgggggagacttagtgaagcctggagggtccctgaaactctcctgtgcagcctctggattcactttcagtaactatggcttgtcttggattcgccagactccagacaagaggctggagtgggtcgcaaccattagtagtggtggtagttacacctactatcctgacagtgtgaaggggcgattcaccatctccagagacagtgccaagaacaccctgtacctgcaaatgagcagtctgaagtctgaggacacagccatgtattactgtgcaagacatgcgcattactacggtgttagcccgtactactttgactactggggccaaggcacctgtctcacagtctcctca

SEQ ID NO: 282 - DNA, 357 bp, synthetic construct
  misc_feature 1..357: note = F847C.10B9 (847.10B9) VH nucleic acid
  source 1..357: mol_type = other DNA
cagatccagttggtgcagtctggacctgaggtgaagaagcctggagagacagtcaagatctcctgcaaggcttctgggtatatcttcacaaactatggaatgaactgggtgaagcaggctccaggaaagggtttaaagtggatgggctggataaacacctacactggagagccaacatatactgatgacttcaagggacggtttgccttctctttggaaacctctgccagcactgcctatttgcagatcaacaacctcaaaaatgaggacacggctacatatttctgtgcaaagtttggtaactacgtgggagctatggactactggggtcaaggaacctcagtcaccgtctcctca

SEQ ID NO: 283 - DNA, 360 bp, synthetic construct
  misc_feature 1..360: note = F847C.11B1 (847.11B1) VH nucleic acid
  source 1..360: mol_type = other DNA
gaggtccagcttcagcagtctggacctgagctggtgaaacctggggcctcagtgaagatatcctgcaaggcttctggaaacacattcactgaccacaacatgcactgggtgaagcagagccatggaaagagccttgagtggattggatatatttatccttacaatggtggtactggctacaaccagaagttcaagagcaaggccacattgactgtagacaattcctccagcacagtctacatggagctccgcagcctgacatctgaggactctgcagtctattactgtgcaagaggggagtatgattacctggcctggtttgcttactggggccaagggactctggtcactgtctctgca

SEQ ID NO: 284 - DNA, 357 bp, synthetic construct
  misc_feature 1..357: note = F847C.12F12 (847.12F12) VH nucleic acid
  source 1..357: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttgcggatcaacaacctcaaaaatgaggacacggctacatatttctgtgcaaagtttggtaactacgtgggagctatggactactggggtcaaggaacctcagtcaccgtctcctca

SEQ ID NO: 285 - DNA, 357 bp, synthetic construct; identical to SEQ ID NO: 282
  misc_feature 1..357: note = F847C.26F5 (847.26F5) VH nucleic acid
  source 1..357: mol_type = other DNA

SEQ ID NO: 286 - DNA, 351 bp, synthetic construct
  misc_feature 1..351: note = F847C.4B10 (847.4B10) VH nucleic acid
  source 1..351: mol_type = other DNA
gaggtgcagcttgttgagactgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtacaaggtttactacggtagtagagatggactactggggtcaaggaacctcagtcaccgtctcctca

SEQ ID NO: 287 - DNA, 336 bp, synthetic construct
  misc_feature 1..336: note = F849C.8D10 (849.8D10) VH nucleic acid
  source 1..336: mol_type = other DNA
caggtccaactgcagcagactggtgctgagcttgtgaagcctggggcctcagtgaagctgtcctgcaaggcttctggctatactttcaccagctactggataaactgggtgaagcagaggcctggacaaggccttgagtggattggaaacatttatcctggtagtagtagtatttactacagtgagaagttcaagagtaaggccacactgactgtagacacatcctccagcacagcctacatgcagctcagcagcctgacatctgacgactctgcggtctattattgtgcaagatggggctactggggccaaggcaccactctcacagtctcctca

SEQ ID NO: 288 - DNA, 363 bp, synthetic construct
  misc_feature 1..363: note = F849C.8H3 VH nucleic acid
  source 1..363: mol_type = other DNA
caggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataaacactataacccagccctgaagagccggctcacaatctccaaggatacctccaaaaaccaggtcttcctcaggatcgccaatgtggacactgcagataatgccacatattactgtgctcgaatcgactatggtgactacgtcgggtttgcttattggggccaagggactctggtcactgtctctgca

SEQ ID NO: 289 - DNA, 336 bp, synthetic construct
  misc_feature 1..336: note = TB001 (IMT001-4) VL nucleic acid
  source 1..336: mol_type = other DNA
gacatcgtgctgacccagtctccactgagcctgcctgttacacctggcgagccagcctccatctcctgcagatcctctaagtccctgctgtacaaggacggcaagacctacctgaactggttcctgcagaagcccggccagtctcctcagctcctgatctacctgatgagcacccatgcctctggcgtgcccgatagattttccggctctggctctggcaccgacttcaccctgaagatctccagagtggaagccgaggacgtgggcgtgtactactgtcagcagctggtggactaccctctgacctttggcggcggaacaaagctggaaatcaag

SEQ ID NO: 290 - DNA, 336 bp, synthetic construct
  misc_feature 1..336: note = TB006(4A11.H3L1) VL nucleic acid
  source 1..336: mol_type = other DNA
gatatagtgatgacccaaacacctttgtccctgtcagtcaccccaggccaacccgcatctatctcatgcaaatcctcaaaaagtcttttgcactctgacgggataacatatctctactggtatcttcaaaaacctggtcagtccccccagctcttgatctatagaatgagtaaccttgcctctggtgtccccgaccggtttagtggaagcgggtctgggaccgacttcaccctcaagattagtcgcgtcgaagccgaagatgtgggagtgtattactgtgcccagatgctcgaatttcccctgacatttggtcaaggaactaaattggagattaag

SEQ ID NO: 291 - DNA, 321 bp, synthetic construct
  misc_feature 1..321: note = 12G5.D7 VL nucleic acid
  source 1..321: mol_type = other DNA
gacatcctgatgacccaatctccatcctccatgtctgtatctctgggagacacagtcagcatcacttgccatgcaagtcagggcattaactctaatatggggtggttgcagcagaaaccagggaaatcatttaagggcctgatctatcatgcaaccaacttggaagatggagttccatcaaggttcagtggcagtggatctggagcagattattctctcaccatcagcagcctggaatctgaagattttgcagactattactgtgtacagtatgctcagtttcctcctacgttcggatcggggaccaagctggaaataaaa

SEQ ID NO: 292 - DNA, 336 bp, synthetic construct
  misc_feature 1..336: note = 13A12.2E5 VL nucleic acid
  source 1..336: mol_type = other DNA
gatgttgtgatgacccagactccactcactttgtcggttgccattggacaaccagcttccatctcttgcaagtcgagtcagagcctcttatatactaatggaaaaacctatttgaattggttattacagaggccaggccagtctccaaaacgcctaatctatctgctgtctaaattggactctggagtccctgacaggttcagtgccagtggatcagggacagatttcacactgaaaatcagcagagtggaggctgaggatttgggagtttattactgcttgcagagtacacattttccgctcacgttcggtgctgggaccaagctggagatgaaa

SEQ ID NO: 293 - DNA, 336 bp, synthetic construct
  misc_feature 1..336: note = 14H10.2C9 (14H10) VL nucleic acid
  source 1..336: mol_type = other DNA
gatgttgtgatgacccagactccactcactttgtcggttaccattggacaaccagcctccatctcttgcaagtcaagtcagagcctcttcgatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctctcacgttcggtgctgggaccaagctggaaatgaaa

SEQ ID NO: 294 - DNA, 336 bp, synthetic construct
  misc_feature 1..336: note = 15F10.2D6 (15F10) VL nucleic acid
  source 1..336: mol_type = other DNA
gatgttttgatgacccaaactccactctccctgcctgtcagtcttggagatcaagcctccatctcttgcagatctagtcagagcattgtacataataatggaaacacctatttagaatggtacctgcagaaaccaggccagtctccaaaactcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtggtgggaccaagttggagctgaaa

SEQ ID NO: 295 - DNA, 321 bp, synthetic construct
  misc_feature 1..321: note = 19B5.2E6 (19B5) VL nucleic acid
  source 1..321: mol_type = other DNA
gatatacaaatgactcaaactacctcttctctttccgcatctttgggtgatcgggtcactatatcctgctccgcctctcaaggcataaacaattacctgaattggtatcagcaaaaaccagacggaactgtaaaattgcttatctactacgcttcaagtctgcatagtggggttcccagcagattttccggttccgggtccggcactgattattctctcacaattagtaacctggaacctgaggacattgcaacttattactgtcaacaatacagtcaggtaccttacactttcggatcaggaacaaagttggaaatcaag

SEQ ID NO: 296 - DNA, 321 bp, synthetic construct
  misc_feature 1..321: note = 20D11.2C6 (20D11) VL nucleic acid
  source 1..321: mol_type = other DNA
gacatccagatgaaccagtctccatccagtctgtctgcatccctcggagacacaattaccatcacttgccgtgccagtcagaacatttatatttggttaagttggtaccagcagaaaccaggaaatattcctaaactcttgatctataaggcttccaacttgcacacaggcgtcccatcaaggtttagtggcagtggatctggaacagatttcacattaaccatcagcactctgcagcctgaagacattgccacttacttctgtctccagggtcaaagttatcctctcacgttcggtgctgggaccaagctggagatgaaa

SEQ ID NO: 297 - DNA, 321 bp, synthetic construct
  misc_feature 1..321: note = 20H5.A3 (20H5) VL nucleic acid
  source 1..321: mol_type = other DNA
gatatccagatgacacagactacatcctccctgtctgcctctctgggagacagagtcaccatcaattgcagtgcaagtcaggacattagcaattatttaaactggtatcagcagaaaccagatggaactgttaaactcctgatctattacacatcaagtttactctcaggagtcccatcaaggttcagtggcagtgggtctgggacagattattctctcaccatcagcaacctggaacctgaagatattgccacttacttttgtcagcaatatagtaagcttccctatacgttcggatcggggacccacctggaaataaaa

SEQ ID NO: 298 - DNA, 321 bp, synthetic construct
  misc_feature 1..321: note = 23H9.2E4 (23H9) VL nucleic acid
  source 1..321: mol_type = other DNA
gatatccagatgacacagactacatcctccctgtctgcctctctgggagacagagtcaccatcagttgcagtgcaagtcagggcattaacaattatttaaactggtatcagcagaaaccagatggaactgttaaactcctgatctattacgcatcaagtttacactcaggagtcccatcaaggttcagtggcagtgggtctgggacagattattctctcaccatcagcaacctggaacctgaagatattgccacttactattgtcagcagtatagtcaggttccgtatacgttcggatcggggaccaagttggaaataaaa

SEQ ID NO: 299 - DNA, 336 bp, synthetic construct
  misc_feature 1..336: note = 2D10.2B2 (2D10) VL nucleic acid
  source 1..336: mol_type = other DNA
gacatagtaatgacacaggccgcatttagcaatccagtaaccttgggcacatccgcctccatatcttgcaggtcatccaaaagcttgctgcactctgacggtattacttacctgtactggtatttgcagcgacctgggcaatcccctcaactcctgatttaccgcatgagcaacctggcctcaggggtgcctgaccgcttttcaggatcagggagcggcactgacttcaccttgcgaatcagcagagtagaagcagaggatgttggagtttactactgtgcccagatgatcgaatttccacttacttttggggccgggaccattctggagcttaaa

SEQ ID NO: 300 - DNA, 336 bp, synthetic construct
  misc_feature 1..336: note = 3B11.2G2 (3B11) VL nucleic acid
  source 1..336: mol_type = other DNA
gatgttgtgatgacccagactccactcactttgtcggttgccattggacaaccagcttccatctcttgcaagtcgagtcagagcctcttatatactaatggaaaaacctatttgaattggttattacagaggccaggccagtctccaaaacgcctaatctatctggtgtctaaattggactctggagtccctgacaggttcagtggcagtggatcagggacagatttcacactgaaaatcagcagagtggaggctgaggatttgggagtttattactgcttgcagagtacacattttccgctcacgttcggtgctgggaccaagctggagctgaaa

SEQ ID NO: 301 - DNA, 336 bp, synthetic construct
  misc_feature 1..336: note = 7D8.2D8 (7D8) VL nucleic acid
  source 1..336: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 302 - DNA, 336 bp, synthetic construct
  misc_feature 1..336: note = F846C.1B2 VL nucleic acid
  source 1..336: mol_type = other DNA
aacatttggttgacacagtcgccatcatctctggctgtgtctgcaggagaaatggtcactatgacctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagacttttctcttaccatcagcagtgtacaagctgaagacctggcagtttattactgtcatcaatatctctcctcgctcacgttcggtgctgggaccaagctggagctgaaa

SEQ ID NO: 303 - DNA, 336 bp, synthetic construct
  misc_feature 1..336: note = F846C.1F5 (846.1F5) VL nucleic acid
  source 1..336: mol_type = other DNA
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagattttactcttaccatcagcagtgtacaacctgaagacctggcagtttattactgtcatcaatacctctcctcgctcacgttcggtgctgggaccaagctggagctgaaa

SEQ ID NO: 304 - DNA, 336 bp, synthetic construct
  misc_feature 1..336: note = F846C.1H12 (846.1H12) VL nucleic acid
  source 1..336: mol_type = other DNA
aacattatgatgacacagtcgccatcatctctggctgtgtctgcaggacaaaaggtcactatgagctgtaagtccagtcaaagtgttttatacagttcaagtcagaagaactacttggcctggtaccagcagaaaccagggcagtctcctaaacttctgatctactgggcatccactagggaatctggtgtccctgatcgcttctcaggcagtggatctgggacagattttactcttaccatcagcagtgtacaacctgaagacctggcagtttattactgtcatcaatacctctcctcgctcacgttcggtgctgggaccaagctggagctgaaa

SEQ ID NO: 305 - DNA, 336 bp, synthetic construct
  misc_feature 1..336: note = F846C.1H5 (846.1H5) VL nucleic acid
  source 1..336: mol_type = other DNA
gatgttgtgatgacccaaactccactctccctgcctgtcagtcttggagatcaagcctccatctcttgcagatctagtcagagccttgtacacaatagtggaaacacctatttacattggtacctgcagaggccaggccagtctccaaacctcctgatctacaaagtttccaaccgattttctggggtcccagacaggttcagtggcagtggatcagggacagatttcacactcaagatcagcagagtggcggctgaggatctgggagtttatttctgctctcaaagtacacatgttcctccgacgttcggtggaggcaccaagctggaaatcaaa

SEQ ID NO: 306 - DNA, 336 bp, synthetic construct; identical to SEQ ID NO: 303
  misc_feature 1..336: note = F846C.2H3 (846.2H3) VL nucleic acid
  source 1..336: mol_type = other DNA

SEQ ID NO: 307 - DNA, 327 bp, synthetic construct
  misc_feature 1..327: note = F846TC.14A2 (846T.14A2) VL nucleic acid
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtatcaataaccgagttccaggtgttcctgccagattctcaggctccctgattggagacaaggcagccctcaccatcacaggggcacagactgaggatgaggcaatatatttctgtgctctatggtatagcaaccattgggtgttcggtggaggaaccaaactgactgtccta

SEQ ID NO: 308 - DNA, 336 bp, synthetic construct
  misc_feature 1..336: note = F846TC.14E4 (846T.14E4) VL nucleic acid
  source 1..336: mol_type = other DNA
gatattgtgatgacgcaggctgcattctccaatccagtcactcttggaacatcagcttccatctcctgcaggtctagtaagagtctcctacatactaatggcatcacttttttgtattggtatctgcagaagccaggccagtctcctcagcccctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccttggacgttcggtggaggcaccaagctggaaatcaat

SEQ ID NO: 309 - DNA, 336 bp, synthetic construct
  misc_feature 1..336: note = F846TC.16B5 (846T.16B5) VL nucleic acid
  source 1..336: mol_type = other DNA
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactactgatctactgggcatccactagggaatctggtgtccctgatcgcttcacaggcagtggatctgggacagattttactcttaccatcagcagtgtacaagctgaagacctggcagtttattactgtcatcaatacctctcctcgctcacgttcggtgctgggaccaagctggagctgaaa

SEQ ID NO: 310 - DNA, 339 bp, synthetic construct
  misc_feature 1..339: note = F846TC.7F10 (846T.7F10) VL nucleic acid
  source 1..339: mol_type = other DNA
gacattgtgatgacacagtctccatcctccctggctatgtcagtaggacagaaggtcactatgagctgcaagtccagtcagagccttttaaatagtagcaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattatagcactccgtacacgttcggagggggggccaagctggaaataaaa

SEQ ID NO: 311 - DNA, 321 bp, synthetic construct
  misc_feature 1..321: note = F847C.10B9 (847.10B9) VL nucleic acid
  source 1..321: mol_type = other DNA
gacatccggatgactcagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtacacgttcggaggggggaccaagctggagctgaaa

SEQ ID NO: 312 - DNA, 321 bp, synthetic construct
  misc_feature 1..321: note = F847C.11B1 (847.11B1) VL nucleic acid
  source 1..321: mol_type = other DNA
gaaattgtgttgacccagtctccagcatccctgtccgtgactacaggagaaaaagtcactatcagatgcataaccagcattgatattgatgatgatatgaactggtaccagcagaagccaggggaacctcctaagctccttatttcagaaggcaatactcttcgtcctggagtcccatcccgattctccagcagtggctatggcacagattttgtttttacaattgaaaacacgctctcagaagatgttgcagattactactgtttgcaaagtgataacaagcctctcacgttcggtgctgggaccaagctggagctgaaa

SEQ ID NO: 313 - DNA, 321 bp, synthetic construct
  misc_feature 1..321: note = F847C.12F12 (847.12F12) VL nucleic acid
  source 1..321: mol_type = other DNA
gacatccaggtgattcagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtacacgttcggaggggggaccaagctggagctgaaa

SEQ ID NO: 314 - DNA, 321 bp, synthetic construct
  misc_feature 1..321: note = F847C.26F5 (847.26F5) VL nucleic acid
  source 1..321: mol_type = other DNA
gaaattaagatgaaccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctacagcctgaagacattgccacttactactgtcaacagggtcaaagttatccgtacacgttcggaggggggaccaagctggagctgaaa

SEQ ID NO: 315 - DNA, 327 bp, synthetic construct
  misc_feature 1..327: note = F847C.4B10 (847.4B10) VL nucleic acid
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttagtgttcggtggaggaaccaaactgactgtccta

SEQ ID NO: 316 - DNA, 336 bp, synthetic construct
  misc_feature 1..336: note = F849C.8D10 (849.8D10) VL nucleic acid
  source 1..336: mol_type = other DNA
gatgttttgatgacccaaactccactctccctgcctgtcagtcttggagatcaagcctccatctcttgcagatctagtcaggacattgtgcatagtagtggaaacacctatttagaatggtacctgcagaaaccaggccagtctctaaagctcctgatttacaaagtttccaaccgattttctggggtcccagacaggttcagtgccagtggatcagggacagatttcacactcaagatcagcagagtggaggctgaggatctgggagtttattactgctttcaaggttcacatgttcctcccacgttcggctcggggacaaagttggaaataaaa

SEQ ID NO: 317 - DNA, 336 bp, synthetic construct
  misc_feature 1..336: note = F849C.8H3 VL nucleic acid
  source 1..336: mol_type = other DNA
gatattgggatgaggcaggctgcattctccaatccagtcactcttggaacatcagcttccatctcctgcaggtctagtaagagtctcctatatagtaatggcatcacttatttgtattggtttctccagaagccaggccagtctcctcagctcctgatttatcagatgtccaaccttgcctcaggagtcccagacaggttcagtagcagtgggtcaggaactgatttcacactgagaatcagcagagtggaggctgacgatgtgggtgtttattactgtgctcaaaatctagaacttccgtggacgttcggtggaggcaccaagctggaaatcaaa

SEQ ID NO: 318 - DNA, 1332 bp, synthetic construct
  misc_feature 1..1332: note = TB001 (IMT001-4) HC nucleic acid
  source 1..1332: mol_type = other DNA
caggtgcagctggtgcagtccggctccgagctgaaaaaacccggcgcctccgtgaaagtgtcctgcaaagcctccggctacacattcacaaactacggcatgaactgggtgaggcaggcccccggccagggcctgaaatggatgggctggatcaacacaaacacaggcgagcccacatacgtggaggagttcacaggcaggttcgtgttctccctggagacatccgtgtccacagcctacctgcagatctcctccctgaaagccgaggacacagccgtgtacttctgcgccccctacgacaacttcttcgcctactggggccagggcacaacagtgacagtgtcctccgcttccaccaagggacccagcgtgttccctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttcctctagcctgggcaccaagacctacacctgtaatgtggaccacaagccttccaacaccaaggtggacaagcgcgtggaatctaagtacggccctccttgtcctccatgtcctgctccagagtttctcggcggaccctccgtgttcctgtttcctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggactccgacggcagcttcttcctgtattcccgcctgaccgtggacaagtccagatggcaagagggcaacgtgttcagctgctccgtgatgcacgaggccctgcacaatcactacacccagaagtccctgtctctgtccctgggctgatga

SEQ ID NO: 319 - DNA, 1341 bp, synthetic construct
  misc_feature 1..1341: note = TB006(4A11.H3L1) HC nucleic acid
  source 1..1341: mol_type = other DNA
cagattcaattggttcagagcggctcagaactgaaaaagcctggagccagcgttaaagtgagctgtaaagccagtggatacacattcaccacttacgtcatgtcctgggttagacaggcccctggacaggggctggaatggatgggctggataaacactcactctggagtgcccacatacgccgacgactttacaggcaggttcgtgttttccctagatactagtgtgaatactgcatacttgcaaatctcttctctgaaagcagaggataccgccgtgtacttttgcacccgcgatgggaatgatggggatgccatggacaactggggccaggggacgactgtgacagtgtcttcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 320 - DNA, 1350 bp, synthetic construct
  misc_feature 1..1350: note = 12G5.D7 HC nucleic acid
  source 1..1350: mol_type = other DNA
caggtgcagctgaagcagtctggggctgagctggtgaggcctggggcttcagtgaagctgtcctgcaaggcttctggctacactttcactgactactatataaactgggtgaagcagaggcctggacagggacttgagtggattgcaaggatttatcctggaactggtaatactgactacaatgagaagttcaagggcagggccacactgactgcagaaaactcctccagcactgcctacatgcagctcagcagtctgacatctgaggactctgctgtctatttctgtgcaagatttgcgtattactacggtagtggagggtactttgactactggggccacggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 321 - DNA, 1341 bp, synthetic construct
  misc_feature 1..1341: note = 13A12.2E5 HC nucleic acid
  source 1..1341: mol_type = other DNA
cagatccagttggtacagtctggacctgagctgaagaagcctggagagacagtcaagatctcctgcaagacttctgggtataaattcaaaacctatgtgatgagctgggtgaaacaggctccaggaaaggctttaaagtggatgggctggataaacacctactctggagtgccaacatatgctgatgacttcaagggacgatttgccttctctttggaaacctctgccagcactgcctatttggagatcatcaacctcaaaaatgaggacacggctacatatttctgtgcaagagatggtaactacggggatcctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 322 - DNA, 1335 bp, synthetic construct
  misc_feature 1..1335: note = 14H10.2C9 (14H10) HC nucleic acid
  source 1..1335: mol_type = other DNA
cagatccagttggtacagtctggacctgagctgaagaagcctggagagacagtcaagatctcctgcaaggcttctgggtataccttcacaacctatggaatgggctgggtgaaacaggctccaggaaaggatttaaagtggatgggctggataaacacctactctggagtgccaacatatgctgatgacttcaagggacggtttgccttctctttggaaacctctgccagcactgcctatttgcagatcagcaacctcaaaaatgaagacacggctacatatttctgttcaaccccttatgaatacgacggggcttactggggccaggggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 323 - DNA, 1323 bp, synthetic construct
  misc_feature 1..1323: note = 15F10.2D6 (15F10) HC nucleic acid
  source 1..1323: mol_type = other DNA
gaagtacagctggtggagtctgggggaggcttagtgaagcctggagggtccctgaaactctcctgtgcagcctctggattcgctttcagttcctatgccatgtcttgggttcgccagactccggaaaagaggctggagtgggtcgcaaccattagtgatggtggtgtttacacctactatacagaccatgtaaagggccgattcaccatctccagagacaatgccgaggacaacctgtacctgcaaatgagccatctgaagtctgaggacacagccatgtattattgtgtgagagatgggggctactggggccaaggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 324 - DNA, 1332 bp, synthetic construct
  misc_feature 1..1332: note = 19B5.2E6 (19B5) HC nucleic acid
  source 1..1332: mol_type = other DNA
cagatccaacttgttcagagcgggcctgaattgaagaaaccaggcgagactgtaaagatctcctgtaaggccagtggctatacattcaccacctacgcaatgagctgggtcaagcaggcacccggaaagggtctgaagtggatgggttggataaatacatactcaggcgtcccaacctacgctgatgatttgaaggggcgctttgccttttctcttgaaacctcagcatcaaccgcctacttgcagataaataacctcaaaaatgaagacaccgctacctatttctgcgcaagggggccttatgctatggactactggggccagggcacctctgtgactgtaagttctgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 325 - DNA, 1329 bp, synthetic construct
  misc_feature 1..1329: note = 20D11.2C6 (20D11) HC nucleic acid
  source 1..1329: mol_type = other DNA
gaggtccagctgcaacaatctggacctgaactggtgaagcctggggcttcagtgaagatatcctgtaaggcttctggatacacgttcactgacttctacataaactgggtgaagcagagccatggaaagagccttgagtggattggagatattaatcctaagaatggtggtattaactacaacccgaagttcaagatcaaggccacattgactgtagacaagtcctccagcacatcctacatggacctccgcggcctgacatctgaggactctgcagtctattactgcacctcaggctacggatttccttactggggccaagggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 326 - DNA, 1332 bp, synthetic construct
  misc_feature 1..1332: note = 20H5.A3 (20H5) HC nucleic acid
  source 1..1332: mol_type = other DNA
cagatccagttggtacagtctggacctgagctgaagaagcctggagagtcagtcaagatctcctgcaaggcttctggttatgccttcacaacctatggaatgagctgggtgcaacaggctccaggaaagggtttaaagtggatgggctgggtaaacacctactctggagtgccaacatgtgctgatgacttcaagggacggtttgccttctctttggaaacctctgccagcactgcctatttgcagatcaacaacctcagaaatgaggacacggctacatatttctgtgcaagagggccctatgctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 327 - DNA, 1332 bp, synthetic construct
  misc_feature 1..1332: note = 23H9.2E4 (23H9) HC nucleic acid
  source 1..1332: mol_type = other DNA
cagatccagttggtacagtctggacctgagctgaagaagcctggagagacagtcaagatctcctgcaaggcttctgggtataccttcacaacctatgcaatgagctgggtgaaacaggctccaggaaagggtttaaagtggatgggctggataaacacctactctggagtgccaacatatgctgatgacctcaagggacggtttgccttctctttggaaacctctgccagcactgcctatttgcagatcaacaacctcaaaaatgaggacacggctacatatttctgtgcaagagggccctatgctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 328 - DNA, 1344 bp, synthetic construct
  misc_feature 1..1344: note = 2D10.2B2 (2D10) HC nucleic acid
  source 1..1344: mol_type = other DNA
caagtacagctccagcaatcaggacctgaacttgttaagccaggtgcaagcgtcaaaataagttgtaaagcatcaggttacacctttacagactactatatcaactgggtcaagcaacggcctggtcagggacttgaatggataggttggatctaccctgggtcacaagacaccaagtacaatgaaaaatttaaggggaaagccactctgacagtagatactagctcaagtaccgcctacatgcagctcggcagcctcacttccgaggactctgccgtatatttctgtgccaactacttcgggtcttctgggtggttctttgatgtgtggggaaccggcaccaccgtaaccgtttcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 329 - DNA, 1341 bp, synthetic construct
  misc_feature 1..1341: note = 3B11.2G2 (3B11) HC nucleic acid
  source 1..1341: mol_type = other DNA
cagatccagttggtacagtctggacctgagctgaagaagcctggagagacagtcaagatctcctgcaagacttctgggtataaattcaaaacctatgtaatgagctgggtgaaacaggctccaggaaaggctttaaagtggatgggctggataaacacctactctggagtgccaacatatgctgatgacttcaagggacggtttgccttctctttggaaacctctgccagcactgcctatttggagatcatcaacctcaaaaatgaggacacggctacatatttctgtgcaagagatggtaattacggggatcctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 330 - DNA, 1323 bp, synthetic construct
  misc_feature 1..1323: note = 7D8.2D8 (7D8) HC nucleic acid
  source 1..1323: mol_type = other DNA
gaagtgcagctggtggagtctgggggaggcttagtgaagcctggagggtccctgaaactctcctgtgcagcctctggattcactttcagtagctatgccatgtcttgggttcgccagactccggaaaagaggctggagtgggtcgcaaccatcagtgatggtggtatttacacctactatccagacaatgtaaagggccgattcaccatttccagagacaatgccaagaacaacctgttcctgcaaatgagccatctgaagtctgaggatacagccatgtattactgtgtaagagatgggggctactggggccaaggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 331 - DNA, 1350 bp, synthetic construct
  misc_feature 1..1350: note = F846C.1B2 HC nucleic acid
  source 1..1350: mol_type = other DNA
cagatccagttggtgcagtctggacctgagctgaagaagcctggagagacagtcaagatctcctgcaaggcttctgggtataccttcacaaactatggaatgaattgggtgaagcaggctccaggaaagggtttaaagtggatgggctggataaacacctacactggagagccatcatatgctgatgacttcaagggacggtttgccttctctttggaaacctctgccagcactgcctatttgcagatcaacaacctcaaaaatgaggacatggctacatatttctgtgcaagatgggggggttacgctggggattactatgctatggacttctggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 332 - DNA, 1350 bp, synthetic construct
  misc_feature 1..1350: note = F846C.1F5 (846.1F5) HC nucleic acid
  source 1..1350: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccatcatatgctgatgacttcaagggacggtttgccttctctttggaaacctctgccagcactgcctatttgcagatcaacaacctcaaaaatgaggacatggctacatatttctgtgcaagatgggggggttacgatggggattactatgctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 333 - DNA, 1350 bp, synthetic construct
  misc_feature 1..1350: note = F846C.1H12 (846.1H12) HC nucleic acid
  source 1..1350: mol_type = other DNA
cagatccagttggtgcagtctggacctgagctgaagaagcctggagagacagtcaagatctcctgcaaggcttctgggtataccttcacaaactttggaatgaactgggtgaagcaggctccaggaaagggtttaaagtggatgggctggataaacacctacactggagagccatcatatgctgatgacttcaagggacggtttgccttctctttggaaacctctgccagcactgcctatttgcagatcaacaacctcaaaaatgaggacattgctacatatttctgtgcaagatgggggggttacgctggggattactatgctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 334 - DNA, 1332 bp, synthetic construct
  misc_feature 1..1332: note = F846C.1H5 (846.1H5) HC nucleic acid
  source 1..1332: mol_type = other DNA
gaggtccagctgttggagtctgggggaggcttagtgaagcctggagggtccctgaaactctcctgtgcagcctctggattcactttcagcagctatgccatgtcttgggttcgccagactccagagaagaggctggagtgggtcgcatccattagtagtggtggtgacacctactatccagacagtgtgaagggccgattcaccatctctagagataatgccaggaacatcctgtacctgcaaatgagcagtctgaggtctgaggacacggccatgtattactgtgcaagttatggtaactccctctttgactactggggccaaggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 335 - DNA, 1350 bp, synthetic construct; identical to SEQ ID NO: 332
  misc_feature 1..1350: note = F846C.2H3 (846.2H3) HC nucleic acid
  source 1..1350: mol_type = other DNA

SEQ ID NO: 336 - DNA, 1326 bp, synthetic construct
  misc_feature 1..1326: note = F846TC.14A2 (846T.14A2) HC nucleic acid
  source 1..1326: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctggtgacctcaagggacggtttgccttctctttggaaacctctgccagcactgcctatttgcagatcaacaacctcaaaaatgaggacacggctacatatttctgtgtaagatatactatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 337 - DNA, 1356 bp, synthetic construct
  misc_feature 1..1356: note = F846TC.14E4 (846T.14E4) HC nucleic acid
  source 1..1356: mol_type = other DNA
caggttactctgaaagagtctggccctgggatattgcagccctcccagaccctcagtctgacttgttctttctctgggttttcactgagcacttctggtatgggtgtaggctggattcgtcagccttcagggaagggtctggagtggctggcacacatttggtgggatgatgataagtactataacacagccctgaagagcgggctcacaatctccaaggatgcctccaaaaaccaggtcttcctcaagatcgccagtatggacactgcagatactgccacatactactgtgctcgaaacctctatgatggttcctacgggtactatgctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 338 - DNA, 1350 bp, synthetic construct
  misc_feature 1..1350: note = F846TC.16B5 (846T.16B5) HC nucleic acid
  source 1..1350: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccagcatatgctgatgacttcaagggacggtttgccttctctttggaaacctctgccagcactgcctatttgcagatcaacaacctcaaaaatgaggacatggctacatatttctgtgcaagatgggggggttacgatggggattactatgctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 339 - DNA, 1353 bp, synthetic construct
  misc_feature 1..1353: note = F846TC.7F10 (846T.7F10) HC nucleic acid
  source 1..1353: mol_type = other DNA
gaagtgcagctggtggagtctgggggagacttagtgaagcctggagggtccctgaaactctcctgtgcagcctctggattcactttcagtaactatggcttgtcttggattcgccagactccagacaagaggctggagtgggtcgcaaccattagtagtggtggtagttacacctactatcctgacagtgtgaaggggcgattcaccatctccagagacagtgccaagaacaccctgtacctgcaaatgagcagtctgaagtctgaggacacagccatgtattactgtgcaagacatgcgcattactacggtgttagcccgtactactttgactactggggccaaggcacctgtctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 340 - DNA, 1341 bp, synthetic construct
  misc_feature 1..1341: note = F847C.10B9 (847.10B9) HC nucleic acid
  source 1..1341: mol_type = other DNA
cagatccagttggtgcagtctggacctgaggtgaagaagcctggagagacagtcaagatctcctgcaaggcttctgggtatatcttcacaaactatggaatgaactgggtgaagcaggctccaggaaagggtttaaagtggatgggctggataaacacctacactggagagccaacatatactgatgacttcaagggacggtttgccttctctttggaaacctctgccagcactgcctatttgcagatcaacaacctcaaaaatgaggacacggctacatatttctgtgcaaagtttggtaactacgtgggagctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 341 - DNA, 1344 bp, synthetic construct
  misc_feature 1..1344: note = F847C.11B1 (847.11B1) HC nucleic acid
  source 1..1344: mol_type = other DNA
gaggtccagcttcagcagtctggacctgagctggtgaaacctggggcctcagtgaagatatcctgcaaggcttctggaaacacattcactgaccacaacatgcactgggtgaagcagagccatggaaagagccttgagtggattggatatatttatccttacaatggtggtactggctacaaccagaagttcaagagcaaggccacattgactgtagacaattcctccagcacagtctacatggagctccgcagcctgacatctgaggactctgcagtctattactgtgcaagaggggagtatgattacctggcctggtttgcttactggggccaagggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 342 - DNA, 1341 bp, synthetic construct
  misc_feature 1..1341: note = F847C.12F12 (847.12F12) HC nucleic acid
  source 1..1341: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgttgatgacttcaagggacggtttgccttctctttggaaacctctgccagcactgcctatttgcggatcaacaacctcaaaaatgaggacacggctacatatttctgtgcaaagtttggtaactacgtgggagctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 343 - DNA, 1341 bp, synthetic construct; identical to SEQ ID NO: 340
  misc_feature 1..1341: note = F847C.26F5 (847.26F5) HC nucleic acid
  source 1..1341: mol_type = other DNA

SEQ ID NO: 344 - DNA, 1335 bp, synthetic construct
  misc_feature 1..1335: note = F847C.4B10 (847.4B10) HC nucleic acid
  source 1..1335: mol_type = other DNA
gaggtgcagcttgttgagactgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtacaaggtttactacggtagtagagatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 345 - DNA, 1320 bp, synthetic construct
  misc_feature 1..1320: note = F849C.8D10 (849.8D10) HC nucleic acid
  source 1..1320: mol_type = other DNA
caggtccaactgcagcagactggtgctgagcttgtgaagcctggggcctcagtgaagctgtcctgcaaggcttctggctatactttcaccagctactggataaactgggtgaagcagaggcctggacaaggccttgagtggattggaaacatttatcctggtagtagtagtatttactacagtgagaagttcaagagtaaggccacactgactgtagacacatcctccagcacagcctacatgcagctcagcagcctgacatctgacgactctgcggtctattattgtgcaagatggggctactggggccaaggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 346 - DNA, 1347 bp, synthetic construct
  misc_feature 1..1347: note = F849C.8H3 HC nucleic acid
  source 1..1347: mol_type = other DNA
caggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataaacactataacccagccctgaagagccggctcacaatctccaaggatacctccaaaaaccaggtcttcctcaggatcgccaatgtggacactgcagataatgccacatattactgtgctcgaatcgactatggtgactacgtcgggtttgcttattggggccaagggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 347 - DNA, 663 bp, synthetic construct
  misc_feature 1..663: note = TB001 (IMT001-4) LC nucleic acid
  source 1..663: mol_type = other DNA
gacatcgtgctgacccagtctccactgagcctgcctgttacacctggcgagccagcctccatctcctgcagatcctctaagtccctgctgtacaaggacggcaagacctacctgaactggttcctgcagaagcccggccagtctcctcagctcctgatctacctgatgagcacccatgcctctggcgtgcccgatagattttccggctctggctctggcaccgacttcaccctgaagatctccagagtggaagccgaggacgtgggcgtgtactactgtcagcagctggtggactaccctctgacctttggcggcggaacaaagctggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatga

SEQ ID NO: 348 - DNA, 660 bp, synthetic construct
  misc_feature 1..660: note = TB006(4A11.H3L1) LC nucleic acid
  source 1..660: mol_type = other DNA
gatatagtgatgacccaaacacctttgtccctgtcagtcaccccaggccaacccgcatctatctcatgcaaatcctcaaaaagtcttttgcactctgacgggataacatatctctactggtatcttcaaaaacctggtcagtccccccagctcttgatctatagaatgagtaaccttgcctctggtgtccccgaccggtttagtggaagcgggtctgggaccgacttcaccctcaagattagtcgcgtcgaagccgaagatgtgggagtgtattactgtgcccagatgctcgaatttcccctgacatttggtcaaggaactaaattggagattaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 349 - DNA, 645 bp, synthetic construct
  misc_feature 1..645: note = 12G5.D7 LC nucleic acid
  source 1..645: mol_type = other DNA
gacatcctgatgacccaatctccatcctccatgtctgtatctctgggagacacagtcagcatcacttgccatgcaagtcagggcattaactctaatatggggtggttgcagcagaaaccagggaaatcatttaagggcctgatctatcatgcaaccaacttggaagatggagttccatcaaggttcagtggcagtggatctggagcagattattctctcaccatcagcagcctggaatctgaagattttgcagactattactgtgtacagtatgctcagtttcctcctacgttcggatcggggaccaagctggaaataaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 350 - DNA, 660 bp, synthetic construct
  misc_feature 1..660: note = 13A12.2E5 LC nucleic acid
  source 1..660: mol_type = other DNA
gatgttgtgatgacccagactccactcactttgtcggttgccattggacaaccagcttccatctcttgcaagtcgagtcagagcctcttatatactaatggaaaaacctatttgaattggttattacagaggccaggccagtctccaaaacgcctaatctatctgctgtctaaattggactctggagtccctgacaggttcagtgccagtggatcagggacagatttcacactgaaaatcagcagagtggaggctgaggatttgggagtttattactgcttgcagagtacacattttccgctcacgttcggtgctgggaccaagctggagatgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 351 - DNA, 660 bp, synthetic construct
  misc_feature 1..660: note = 14H10.2C9 (14H10) LC nucleic acid
  source 1..660: mol_type = other DNA
gatgttgtgatgacccagactccactcactttgtcggttaccattggacaaccagcctccatctcttgcaagtcaagtcagagcctcttcgatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttcctctcacgttcggtgctgggaccaagctggaaatgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 352 - DNA, 660 bp, synthetic construct
  misc_feature 1..660: note = 15F10.2D6 (15F10) LC nucleic acid
  source 1..660: mol_type = other DNA
gatgttttgatgacccaaactccactctccctgcctgtcagtcttggagatcaagcctccatctcttgcagatctagtcagagcattgtacataataatggaaacacctatttagaatggtacctgcagaaaccaggccagtctccaaaactcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtggtgggaccaagttggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 353 - DNA, 645 bp, synthetic construct
  misc_feature 1..645: note = 19B5.2E6 (19B5) LC nucleic acid
  source 1..645: mol_type = other DNA
gatatacaaatgactcaaactacctcttctctttccgcatctttgggtgatcgggtcactatatcctgctccgcctctcaaggcataaacaattacctgaattggtatcagcaaaaaccagacggaactgtaaaattgcttatctactacgcttcaagtctgcatagtggggttcccagcagattttccggttccgggtccggcactgattattctctcacaattagtaacctggaacctgaggacattgcaacttattactgtcaacaatacagtcaggtaccttacactttcggatcaggaacaaagttggaaatcaagaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 354 - DNA, 645 bp, synthetic construct
  misc_feature 1..645: note = 20D11.2C6 (20D11) LC nucleic acid
  source 1..645: mol_type = other DNA
gacatccagatgaaccagtctccatccagtctgtctgcatccctcggagacacaattaccatcacttgccgtgccagtcagaacatttatatttggttaagttggtaccagcagaaaccaggaaatattcctaaactcttgatctataaggcttccaacttgcacacaggcgtcccatcaaggtttagtggcagtggatctggaacagatttcacattaaccatcagcactctgcagcctgaagacattgccacttacttctgtctccagggtcaaagttatcctctcacgttcggtgctgggaccaagctggagatgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 355 - DNA, 645 bp, synthetic construct
  misc_feature 1..645: note = 20H5.A3 (20H5) LC nucleic acid
  source 1..645: mol_type = other DNA
gatatccagatgacacagactacatcctccctgtctgcctctctgggagacagagtcaccatcaattgcagtgcaagtcaggacattagcaattatttaaactggtatcagcagaaaccagatggaactgttaaactcctgatctattacacatcaagtttactctcaggagtcccatcaaggttcagtggcagtgggtctgggacagattattctctcaccatcagcaacctggaacctgaagatattgccacttacttttgtcagcaatatagtaagcttccctatacgttcggatcggggacccacctggaaataaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 356 - DNA, 645 bp, synthetic construct
  misc_feature 1..645: note = 23H9.2E4 (23H9) LC nucleic acid
  source 1..645: mol_type = other DNA
gatatccagatgacacagactacatcctccctgtctgcctctctgggagacagagtcaccatcagttgcagtgcaagtcagggcattaacaattatttaaactggtatcagcagaaaccagatggaactgttaaactcctgatctattacgcatcaagtttacactcaggagtcccatcaaggttcagtggcagtgggtctgggacagattattctctcaccatcagcaacctggaacctgaagatattgccacttactattgtcagcagtatagtcaggttccgtatacgttcggatcggggaccaagttggaaataaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 357 - DNA, 660 bp, synthetic construct
  misc_feature 1..660: note = 2D10.2B2 (2D10) LC nucleic acid
  source 1..660: mol_type = other DNA
gacatagtaatgacacaggccgcatttagcaatccagtaaccttgggcacatccgcctccatatcttgcaggtcatccaaaagcttgctgcactctgacggtattacttacctgtactggtatttgcagcgacctgggcaatcccctcaactcctgatttaccgcatgagcaacctggcctcaggggtgcctgaccgcttttcaggatcagggagcggcactgacttcaccttgcgaatcagcagagtagaagcagaggatgttggagtttactactgtgcccagatgatcgaatttccacttacttttggggccgggaccattctggagcttaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 358 - DNA, 660 bp, synthetic construct
  misc_feature 1..660: note = 3B11.2G2 (3B11) LC nucleic acid
  source 1..660: mol_type = other DNA
gatgttgtgatgacccagactccactcactttgtcggttgccattggacaaccagcttccatctcttgcaagtcgagtcagagcctcttatatactaatggaaaaacctatttgaattggttattacagaggccaggccagtctccaaaacgcctaatctatctggtgtctaaattggactctggagtccctgacaggttcagtggcagtggatcagggacagatttcacactgaaaatcagcagagtggaggctgaggatttgggagtttattactgcttgcagagtacacattttccgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 359 - DNA, 660 bp, synthetic construct
  misc_feature 1..660: note = 7D8.2D8 (7D8) LC nucleic acid
  source 1..660: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 360 - DNA, 660 bp, synthetic construct
  misc_feature 1..660: note = F846C.1B2 LC nucleic acid
  source 1..660: mol_type = other DNA
aacatttggttgacacagtcgccatcatctctggctgtgtctgcaggagaaatggtcactatgacctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagacttttctcttaccatcagcagtgtacaagctgaagacctggcagtttattactgtcatcaatatctctcctcgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 361 - DNA, 660 bp, synthetic construct
  misc_feature 1..660: note = F846C.1F5 (846.1F5) LC nucleic acid
  source 1..660: mol_type = other DNA
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagattttactcttaccatcagcagtgtacaacctgaagacctggcagtttattactgtcatcaatacctctcctcgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 362 - DNA, 660 bp, synthetic construct
  misc_feature 1..660: note = F846C.1H12 (846.1H12) LC nucleic acid
  source 1..660: mol_type = other DNA
aacattatgatgacacagtcgccatcatctctggctgtgtctgcaggacaaaaggtcactatgagctgtaagtccagtcaaagtgttttatacagttcaagtcagaagaactacttggcctggtaccagcagaaaccagggcagtctcctaaacttctgatctactgggcatccactagggaatctggtgtccctgatcgcttctcaggcagtggatctgggacagattttactcttaccatcagcagtgtacaacctgaagacctggcagtttattactgtcatcaatacctctcctcgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 363 - DNA, 663 bp, synthetic construct
  misc_feature 1..663: note = F846C.1H5 (846.1H5) LC nucleic acid
  source 1..663: mol_type = other DNA
gatgttgtgatgacccaaactccactctccctgcctgtcagtcttggagatcaagcctccatctcttgcagatctagtcagagccttgtacacaatagtggaaacacctatttacattggtacctgcagaggccaggccagtctccaaacctcctgatctacaaagtttccaaccgattttctggggtcccagacaggttcagtggcagtggatcagggacagatttcacactcaagatcagcagagtggcggctgaggatctgggagtttatttctgctctcaaagtacacatgttcctccgacgttcggtggaggcaccaagctggaaatcaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaagga

SEQ ID NO: 364 - DNA, 660 bp, synthetic construct; identical to SEQ ID NO: 361
  misc_feature 1..660: note = F846C.2H3 (846.2H3) LC nucleic acid
  source 1..660: mol_type = other DNA

SEQ ID NO: 365 - DNA, 651 bp, synthetic construct
  misc_feature 1..651: note = F846TC.14A2 (846T.14A2) LC nucleic acid
  source 1..651: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtatcaataaccgagttccaggtgttcctgccagattctcaggctccctgattggagacaaggcagccctcaccatcacaggggcacagactgaggatgaggcaatatatttctgtgctctatggtatagcaaccattgggtgttcggtggaggaaccaaactgactgtcctacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 366 - DNA, 660 bp, synthetic construct
  misc_feature 1..660: note = F846TC.14E4 (846T.14E4) LC nucleic acid
  source 1..660: mol_type = other DNA
gatattgtgatgacgcaggctgcattctccaatccagtcactcttggaacatcagcttccatctcctgcaggtctagtaagagtctcctacatactaatggcatcacttttttgtattggtatctgcagaagccaggccagtctcctcagcccctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacttccttggacgttcggtggaggcaccaagctggaaatcaatcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 367 - DNA, 660 bp, synthetic construct
  misc_feature 1..660: note = F846TC.16B5 (846T.16B5) LC nucleic acid
  source 1..660: mol_type = other DNA
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactactgatctactgggcatccactagggaatctggtgtccctgatcgcttcacaggcagtggatctgggacagattttactcttaccatcagcagtgtacaagctgaagacctggcagtttattactgtcatcaatacctctcctcgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 368 - DNA, 663 bp, synthetic construct
  misc_feature 1..663: note = F846TC.7F10 (846T.7F10) LC nucleic acid
  source 1..663: mol_type = other DNA
gacattgtgatgacacagtctccatcctccctggctatgtcagtaggacagaaggtcactatgagctgcaagtccagtcagagccttttaaatagtagcaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattatagcactccgtacacgttcggagggggggccaagctggaaataaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 369 - DNA, 645 bp, synthetic construct
  misc_feature 1..645: note = F847C.10B9 (847.10B9) LC nucleic acid
  source 1..645: mol_type = other DNA
gacatccggatgactcagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtacacgttcggaggggggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 370 - DNA, 645 bp, synthetic construct
  misc_feature 1..645: note = F847C.11B1 (847.11B1) LC nucleic acid
  source 1..645: mol_type = other DNA
gaaattgtgttgacccagtctccagcatccctgtccgtgactacaggagaaaaagtcactatcagatgcataaccagcattgatattgatgatgatatgaactggtaccagcagaagccaggggaacctcctaagctccttatttcagaaggcaatactcttcgtcctggagtcccatcccgattctccagcagtggctatggcacagattttgtttttacaattgaaaacacgctctcagaagatgttgcagattactactgtttgcaaagtgataacaagcctctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 371 - DNA, 645 bp, synthetic construct
  misc_feature 1..645: note = F847C.12F12 (847.12F12) LC nucleic acid
  source 1..645: mol_type = other DNA
gacatccaggtgattcagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccgtacacgttcggaggggggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 372 - DNA, 646 bp, synthetic construct
  misc_feature 1..646: note = F847C.26F5 (847.26F5) LC nucleic acid
  source 1..646: mol_type = other DNA
gaaattaagatgaaccagtctccatccagtctgtctgcatcccttggagacacaattaccatcacttgccatgccagtcagaacattaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctacagcctgaagacattgccacttactactgtcaacagggtcaaagttatccgtacacgttcggaggggggaccaagctggagctgaaaaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaag

SEQ ID NO: 373 - DNA, 651 bp, synthetic construct
  misc_feature 1..651: note = F847C.4B10 (847.4B10) LC nucleic acid
  source 1..651: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttagtgttcggtggaggaaccaaactgactgtcctaaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 374 - DNA, 660 bp, synthetic construct
  misc_feature 1..660: note = F849C.8D10 (849.8D10) LC nucleic acid
  source 1..660: mol_type = other DNA
gatgttttgatgacccaaactccactctccctgcctgtcagtcttggagatcaagcctccatctcttgcagatctagtcaggacattgtgcatagtagtggaaacacctatttagaatggtacctgcagaaaccaggccagtctctaaagctcctgatttacaaagtttccaaccgattttctggggtcccagacaggttcagtgccagtggatcagggacagatttcacactcaagatcagcagagtggaggctgaggatctgggagtttattactgctttcaaggttcacatgttcctcccacgttcggctcggggacaaagttggaaataaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 375 - DNA, 660 bp, synthetic construct
  misc_feature 1..660: note = F849C.8H3 LC nucleic acid
  source 1..660: mol_type = other DNA
gatattgggatgaggcaggctgcattctccaatccagtcactcttggaacatcagcttccatctcctgcaggtctagtaagagtctcctatatagtaatggcatcacttatttgtattggtttctccagaagccaggccagtctcctcagctcctgatttatcagatgtccaaccttgcctcaggagtcccagacaggttcagtagcagtgggtcaggaactgatttcacactgagaatcagcagagtggaggctgacgatgtgggtgtttattactgtgctcaaaatctagaacttccgtggacgttcggtggaggcaccaagctggaaatcaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 376 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 377 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 378 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 379 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 380 - protein, 47 aa, Homo sapiens
HSGINSSDAEVLTLFNVTEAQSGEYVCKVSNYIGEANQSAWLTVTRP

SEQ ID NO: 381 - protein, 769 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 382 - protein, 821 aa, Homo sapiens
MVSWGRFICLVVVTMATLSLARPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 383 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 384 - protein, 802 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 385 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 386 - protein, 1356 aa, Homo sapiens [sequence omitted]

SEQ ID NO: 387 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 388 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 389 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 390 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 391 - protein, 1255 aa, Homo sapiens [sequence omitted]

SEQ ID NO: 392 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 393 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 394 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 395 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 396 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 397 - protein, 7 aa, synthetic construct
  REGION 1..7: note = VH_CDR1_21
GFSLTSY

SEQ ID NO: 398 - protein, 7 aa, synthetic construct
  REGION 1..7: note = VH_CDR1_22
GYTFTSY

SEQ ID NO: 399 - protein, 7 aa, synthetic construct
  REGION 1..7: note = VH_CDR1_23
GYTFSDY

SEQ ID NO: 400 - protein, 8 aa, synthetic construct
  REGION 1..8: note = VH_CDR2_19
INTNTGES

SEQ ID NO: 401 - protein, 8 aa, synthetic construct
  REGION 1..8: note = VH_CDR2_20
INTHSGVP

SEQ ID NO: 402 - protein, 7 aa, synthetic construct
  REGION 1..7: note = VH_CDR2_21
IWSGGST

SEQ ID NO: 403 - protein, 8 aa, synthetic construct
  REGION 1..8: note = VH_CDR2_22
IDPSDSET

SEQ ID NO: 404 - protein, 8 aa, synthetic construct
  REGION 1..8: note = VH_CDR2_23
IHPNSGST

SEQ ID NO: 405 - protein, 8 aa, synthetic construct
  REGION 1..8: note = VH_CDR2_24
INPNNGGT

SEQ ID NO: 406 - protein, 8 aa, synthetic construct
  REGION 1..8: note = VH_CDR2_25
IYPVSVNT

SEQ ID NO: 407 - protein, 7 aa, synthetic construct
  REGION 1..7: note = VH_CDR3_24
ARYPMDY

SEQ ID NO: 408 - protein, 11 aa, synthetic construct
  REGION 1..11: note = VH_CDR3_25
QPGGVTGTLTT

SEQ ID NO: 409 - protein, 12 aa, synthetic construct
  REGION 1..12: note = VH_CDR3_26
TRDGNDGDAMDN

SEQ ID NO: 410 - protein, 14 aa, synthetic construct
  REGION 1..14: note = VH_CDR3_27
AKGPYDYDLGWFAY

SEQ ID NO: 411 - protein, 8 aa, synthetic construct
  REGION 1..8: note = VH_CDR3_28
ARHGYYDY

SEQ ID NO: 412 - protein, 12 aa, synthetic construct
  REGION 1..12: note = VH_CDR3_29
TRWGIYYYARDY

SEQ ID NO: 413 - protein, 10 aa, synthetic construct
  REGION 1..10: note = VH_CDR3_30
AVPYEYDGAY

SEQ ID NO: 414 - protein, 10 aa, synthetic construct
  REGION 1..10: note = VH_CDR3_31
ATPYEYDGAY

SEQ ID NO: 415 - protein, 5 aa, synthetic construct
  REGION 1..5: note = VH_CDR3_32
AYLDY

SEQ ID NO: 416 - protein, 14 aa, synthetic construct
  REGION 1..14: note = VH_CDR3_33
AKSPDGYDVAWFGY

SEQ ID NO: 417 - protein, 11 aa, synthetic construct
  REGION 1..11: note = VL_CDR1_23
QSLLYSNGKTY

SEQ ID NO: 418 - protein, 11 aa, synthetic construct
  REGION 1..11: note = VL_CDR1_24
QSLLYTDGKTY

SEQ ID NO: 419 - protein, 6 aa, synthetic construct
  REGION 1..6: note = VL_CDR1_25
QSVSND

SEQ ID NO: 420 - protein, 6 aa, synthetic construct
  REGION 1..6: note = VL_CDR1_26
QGISNY

SEQ ID NO: 421 - protein, 6 aa, synthetic construct
  REGION 1..6: note = VL_CDR1_27
QDVSTA

SEQ ID NO: 422 - protein, 6 aa, synthetic construct
  REGION 1..6: note = VL_CDR1_28
QNVGTN

SEQ ID NO: 423 - protein, 11 aa, synthetic construct
  REGION 1..11: note = VL_CDR1_29
QSLFHSDGKTY

SEQ ID NO: 424 - protein, 6 aa, synthetic construct
  REGION 1..6: note = VL_CDR1_30
QNINIW

SEQ ID NO: 425 - protein, 11 aa, synthetic construct
  REGION 1..11: note = VL_CDR1_31
QSLLHSDGKTY

SEQ ID NO: 426 - protein, 6 aa, synthetic construct
  REGION 1..6: note = VL_CDR1_32
QDVRTA

SEQ ID NO: 427-428 - skipped (2 entries)

SEQ ID NO: 429 - protein, 9 aa, synthetic construct
  REGION 1..9: note = VL_CDR3_22
VQGTHFPQT

SEQ ID NO: 430 - protein, 9 aa, synthetic construct
  REGION 1..9: note = VL_CDR3_23
QQDYTSPYT

SEQ ID NO: 431 - protein, 9 aa, synthetic construct
  REGION 1..9: note = VL_CDR3_24
QQYSELPYT

SEQ ID NO: 432 - protein, 9 aa, synthetic construct
  REGION 1..9: note = VL_CDR3_25
QQHYTTPLT

SEQ ID NO: 433 - protein, 9 aa, synthetic construct
  REGION 1..9: note = VL_CDR3_26
EQYSNFPLT

SEQ ID NO: 434 - protein, 9 aa, synthetic construct
  REGION 1..9: note = VL_CDR3_27
QQYSSYPWT

SEQ ID NO: 435 - protein, 114 aa, synthetic construct
  REGION 1..114: note = VH-28
QIQLVQSGPELTKPGETVKISCKASGYTFTDYGMNWVRQAPGETLKWMGWINTYTGEPTYADDFKGRFAFSLESSASTAYLQINNLKNEDVATYFCARYPMDYWGQGTSVTVSS

SEQ ID NO: 436 - protein, 118 aa, synthetic construct
  REGION 1..118: note = VH-29
QIQLVQSGPELKKPGETVKISCKASGYTFTNYGMNWVKQAPGKGLKWMGWINTNTGEPTYAEEFKGRFAFSLETSASTAYLQINNLKNEDTATYFCATGGGNWDFDYWGQGTTLTVSS

SEQ ID NO: 437 - protein, 116 aa, synthetic construct
  REGION 1..116: note = VH-30
QIQLVQSGPELKKPGETVKISCKASGYTFTNYGMNWVKQAPGKGLKWMGWINTNTGEPTYVEEFKGRFAFSLETSASTAYLQINNLKNEDTATYFCAPYDNFFAYWGQGTLVTVSA

SEQ ID NO: 438 - protein, 116 aa, synthetic construct
  REGION 1..116: note = VH-31
QVQLQQSGPELVKPGASVKISCKASGYSFTNYYIHWVKQRPGQGLEWIGWIYPGSGNTNYNEKFKGKATLTADTSSSTTNMQLSSLTSEDSAVYYCSTAPGGFDVWGSGTTVTVSS

SEQ ID NO: 439 - protein, 116 aa, synthetic construct
  REGION 1..116: note = VH-32
QVQLVQSGAEVKKPGASVKVSCKASGYSFTNYYMHWVRQAPGQGLEWMGWIYPGSGNTNYNEKFQGRVTMTADTSISTAYMELSRLRSDDTAVYYCSTAPGGFDVWGQGTTVTVSS

SEQ ID NO: 440 - protein, 116 aa, synthetic construct
  REGION 1..116: note = VH-33
QVQLVQSGAEVKKPGASVKVSCKASGYSFTNYYIHWVRQAPGQRLEWMGWIYPGSGNTNYNEKFQGRVTLTADTSASTTYMELSSLRSEDTAVYYCSTAPGGFDVWGQGTTVTVSS

SEQ ID NO: 441 - protein, 119 aa, synthetic construct
  REGION 1..119: note = VH-34
QIQLVQSGPDLKKPGETVKISCKASGYTFTTYVMSWVKQAPGKDLKWMGWINTHSGVPTYADDFKGRFDFSLETSANTAFLQINNLKNEDTATYFCTRDGNDGDAMDNWGQGTSVTVSS

SEQ ID NO: 442 - protein, 120 aa, synthetic construct
  REGION 1..120: note = VH-35
QVQLKQSGPGLVQPSQSLSITCTVSGFSLTSYGVHWVRQPPGKGLEWLGVIWSGGSTDYNAAFISRLSISKDNSKSQVFFKMNSLQADDTAIYYCAKGPYDYDLGWFAYWGQGTLVTVSA

SEQ ID NO: 443 - protein, 116 aa, synthetic construct
  REGION 1..116: note = VH-36
QIQLVQSGPELKKPGETVKISCKASGYTFTTYGMSWVKQAPGKGLKWMAWINTYSGVPTYADDFKGRFAFSLETSASTAYLQINNLTNEDTATYFCARGPYAMDYWGQGTSVTVSS

SEQ ID NO: 444 - protein, 115 aa, synthetic construct
  REGION 1..115: note = VH-37
QVQLQQPGAELVGPGSSVKLSCKASGYTFTNYWIHWVKQRPLQGLEWIGNIDPSDSETHYNQKFKDKATLTVDKSSSTAYMQLSSLTSEDSAVYYCARHGYYDYWGQGTTLTVSS

SEQ ID NO: 445 - protein, 119 aa, synthetic construct
  REGION 1..119: note = VH-38
QVQLQQPGAELVKPGASVKLSCKASGYTFTSYWMHWVKQRPGQGLEWIGMIHPNSGSTDYNEKFKNKATLNVDKSSSTAYIQLSSLTSEDSAVYYCTRWGIYYYARDYWGQGTTLTVSS

SEQ ID NO: 446 - protein, 117 aa, synthetic construct
  REGION 1..117: note = VH-39
QIQLVQSGPELKKPGETVKISCKASGYTFTTYGMSWVKQAPGKGLKWMGWINTYSGVPTYADDFKGRFAFSLETSASTAYLQINNLKNEDTATYFCAVPYEYDGAYWGQGTLVTVSA

SEQ ID NO: 447 - protein, 115 aa, synthetic construct
  REGION 1..115: note = VH-40
EVQLQQSGPELVKPGASVKISCKASGYTFTDYYMNWVKQSHGKSLEWIGDINPNNGGTNYNQKFKGKATLTVDKSSSTAYMELRSLTSEDSAVYYCTSGYGFPYWGQGTLVTVSA

SEQ ID NO: 448 - protein, 117 aa, synthetic construct
  REGION 1..117: note = VH-41
QIQLVQSGPELKKPGETVKISCKASGYTFTTYGMGWVKQAPGKGLKWMGWINTYSGVPTYADDFKGRFAFSLETSTSTAYLQINNLKNEDMATYFCATPYEYDGAYWGQGTLVTVSA

SEQ ID NO: 449 - protein, 112 aa, synthetic construct
  REGION 1..112: note = VH-42
QVQLQQSGPELVKPGDSVKISCKAAGYTFSDYYINWVKQRPGQGLEWIGWIYPVSVNTKYNEKFKGKATLTVDTSSSTAYMQLSSLTSEDSGVYFCAYLDYWGQGTLVTVSA

SEQ ID NO: 450 - protein, 120 aa, synthetic construct
  REGION 1..120: note = VH-43
QVQLKQSGPGLVQPSQSLSITCTVSGFSLTSYGVHWVRQAPGKGLEWLGVIWSGGSTDYNAAFMSRLSISKDNSKSQVFFKMNSLQADDTAIYYCAKSPDGYDVAWFGYWGQGTLVTVSA

SEQ ID NO: 451 - protein, 112 aa, synthetic construct
  REGION 1..112: note = VL-30
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPQTFGGGTKLEIK

SEQ ID NO: 452 - protein, 112 aa, synthetic construct
  REGION 1..112: note = VL-31
DIVITQDELSNPVTSGESVSISCRSSKSLLYKDGKTYLNWFLQRPGQSPQLLIYLMSTHASGVSDRFSGSGSGTDFTLEISRVKAEDVGVYYCQQLVDYPLTFGAGTKLELK

SEQ ID NO: 453 - protein, 112 aa, synthetic construct
  REGION 1..112: note = VL-32
DIVMTQAAFSNPVTLGTSASISCRSSKSLLHSDGITYLYWYLQRPGQSPQLLIYRMSNLASGVPDRFSGSGSGTDFTLRISRVEAEDVGVYYCAQMLEFPLTFGAGTKLELK

SEQ ID NO: 454 - protein, 112 aa, synthetic construct
  REGION 1..112: note = VL-33
DIVMTQSPLSLPVTPGEPASISCRSSKSLLHSDGITYLYWYLQKPGQSPQLLIYRMSNLASGVPDRFSGSGSGTDFTLKISRVEAEDVGVYYCAQMLEFPLTFGQGTKLEIK

SEQ ID NO: 455 - protein, 113 aa, synthetic construct
  REGION 1..113: note = VL-34
DVVMTQTPLTLSVTIGQPASISCKSSQSLLYTDGKTYLSWFLQRPGQSPKRLIYLVSKLDSGVPDRFSGSGSGTDFTLKISRVEAEDLGVYYCLQSTHFPLTFGAGTKLEVKR

SEQ ID NO: 456 - protein, 108 aa, synthetic construct
  REGION 1..108: note = VL-35
SIVMTQTPKFLLVSAGDRVTITCKASQSVSNDVAWYQQKPGQSPKLLIYYASNRYTGVPDRFTGSGYGTDFTFTISTVQAEDLAVYFCQQDYTSPYTFGGGTKLEIKR

SEQ ID NO: 457 - protein, 108 aa, synthetic construct
  REGION 1..108: note = VL-36
DIQMTQTTSSLSASLGDRVTISCSASQGISNYLNWYQQKPDGTVKLLIYYTSSLHSGVPSRFSGSGSGTDYSLTISNLEPEDIATYYCQQYSELPYTFGSGTKLEIKR

SEQ ID NO: 458 - protein, 108 aa, synthetic construct
  REGION 1..108: note = VL-37
DIVMTQSHKFMSTSVGDRVSITCKASQDVSTAVAWYQQKPGQSPKLLIYWASTRHTGVPDRFTGSGSGTDYTLTISSVQAEDLALYYCQQHYTTPLTFGAGTKLELKR

SEQ ID NO: 459 - protein, 108 aa, synthetic construct
  REGION 1..108: note = VL-38
DIVMTQSQKFMSTSVGERVSITCKASQNVGTNVAWYQQKAGQSLELLIYGASNRHTGVPDRFTGSGSGTDFTLTITNVQSEDMTNYFCEQYSNFPLTFGAGTKLELKR

SEQ ID NO: 460 - protein, 113 aa, synthetic construct
  REGION 1..113: note = VL-39
DVVMTQTPLTLSVTIGQPASISCKSSQSLFHSDGKTYLNWLLQRPGQSPKRLIYLVSKLDSGVPDRFTGSGSGTDFTPKISRVEDEDLGVYYCWQGTHFPLTFGAGTKLEMKR

SEQ ID NO: 461 - protein, 108 aa, synthetic construct
  REGION 1..108: note = VL-40
DIQMNQSPSSLSASLGDTISITCRASQNINIWLSWYQQKPGNIPQLLIYKASNLHTGVPSRFSGSGSGTDFTLTISSLQPEDIATYYCLQGQSYPLTFGAGTKLVMKR

SEQ ID NO: 462 - protein, 111 aa, synthetic construct
  REGION 1..111: note = VL-41
DVVMTQTPLTLSVTIGQPASISCKSSQSLLHSDGKTYLNWLLQRPGQPPKRLMYLVSTLDSGVPDRFTGSGSGTDFTLKISRVEAEDLGVYYCWQGTHFPLTFGAGTKPER

SEQ ID NO: 463 - protein, 111 aa, synthetic construct
  REGION 1..111: note = VL-42
DIVMTQAAFSNPVTLGTSASISCRSSKSLLHSDGITYFYWYLQRPGQSPQLLIYRMSNLASGVPDRFSGSGSGTDFTLRISRVEAEDVGVYYCAQMLEFPLTFGAGTKLER

SEQ ID NO: 464 - protein, 108 aa, synthetic construct
  REGION 1..108: note = VL-43
DIVMTQSHKFMSTSVGDRVSITCKASQDVRTAVAWYQQKPGQSPKLLIYWASTRHTGVPDRFTGSGSGTDFTLTLSNVQSEDLADYFCQQYSSYPWTFGGGTKLEIKR

SEQ ID NO: 465 - protein, 444 aa, synthetic construct
  REGION 1..444: note = HC-30
QVQLQQSGPELVRPGALVKISCKASGYTFTSYDINWVKQRPGQGLEWIGWIYPGDGSTKYNEKFKGKATLTADKSSSTAYMQLSSLTSENSAVYFCAREAASNAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 466 - protein, 448 aa, synthetic construct
  REGION 1..448: note = HC-31
EVKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTRHYGFPYWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 467 - protein, 440 aa, synthetic construct
  REGION 1..440: note = HC-32
QIQLVQSGPELTKPGETVKISCKASGYTFTDYGMNWVRQAPGETLKWMGWINTYTGEPTYADDFKGRFAFSLESSASTAYLQINNLKNEDVATYFCARYPMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 468 - protein, 444 aa, synthetic construct
  REGION 1..444: note = HC-33
QIQLVQSGPELKKPGETVKISCKASGYTFTNYGMNWVKQAPGKGLKWMGWINTNTGEPTYAEEFKGRFAFSLETSASTAYLQINNLKNEDTATYFCATGGGNWDFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 469 - protein, 446 aa, synthetic construct
  REGION 1..446: note = HC-34
QIQLVQSGPELKKPGETVKISCKASGYTFTNYGMNWVKQAPGKGLKWMGWINTNTGEPTYVEEFKGRFAFSLETSASTAYLQINNLKNEDTATYFCAPYDNFFAYWGQGTLVTVSAAKTTPPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 470 - protein, 442 aa, synthetic construct
  REGION 1..442: note = HC-35
QVQLQQSGPELVKPGASVKISCKASGYSFTNYYIHWVKQRPGQGLEWIGWIYPGSGNTNYNEKFKGKATLTADTSSSTTNMQLSSLTSEDSAVYYCSTAPGGFDVWGSGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 471 - protein, 442 aa, synthetic construct
  REGION 1..442: note = HC-36
QVQLVQSGAEVKKPGASVKVSCKASGYSFTNYYMHWVRQAPGQGLEWMGWIYPGSGNTNYNEKFQGRVTMTADTSISTAYMELSRLRSDDTAVYYCSTAPGGFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 472 - protein, 442 aa, synthetic construct
  REGION 1..442: note = HC-37
QVQLVQSGAEVKKPGASVKVSCKASGYSFTNYYIHWVRQAPGQRLEWMGWIYPGSGNTNYNEKFQGRVTLTADTSASTTYMELSSLRSEDTAVYYCSTAPGGFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 473 - protein, 445 aa, synthetic construct
  REGION 1..445: note = HC-38
QIQLVQSGPDLKKPGETVKISCKASGYTFTTYVMSWVKQAPGKDLKWMGWINTHSGVPTYADDFKGRFDFSLETSANTAFLQINNLKNEDTATYFCTRDGNDGDAMDNWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 474 - protein, 446 aa, synthetic construct
  REGION 1..446: note = HC-39
QVQLKQSGPGLVQPSQSLSITCTVSGFSLTSYGVHWVRQPPGKGLEWLGVIWSGGSTDYNAAFISRLSISKDNSKSQVFFKMNSLQADDTAIYYCAKGPYDYDLGWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 475 - protein, 442 aa, synthetic construct
  REGION 1..442: note = HC-40
QIQLVQSGPELKKPGETVKISCKASGYTFTTYGMSWVKQAPGKGLKWMAWINTYSGVPTYADDFKGRFAFSLETSASTAYLQINNLTNEDTATYFCARGPYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 476 - protein, 441 aa, synthetic construct
  REGION 1..441: note = HC-41
QVQLQQPGAELVGPGSSVKLSCKASGYTFTNYWIHWVKQRPLQGLEWIGNIDPSDSETHYNQKFKDKATLTVDKSSSTAYMQLSSLTSEDSAVYYCARHGYYDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 477 - protein, 445 aa, synthetic construct
  REGION 1..445: note = HC-42
QVQLQQPGAELVKPGASVKLSCKASGYTFTSYWMHWVKQRPGQGLEWIGMIHPNSGSTDYNEKFKNKATLNVDKSSSTAYIQLSSLTSEDSAVYYCTRWGIYYYARDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 478 - protein, 443 aa, synthetic construct
  REGION 1..443: note = HC-43
QIQLVQSGPELKKPGETVKISCKASGYTFTTYGMSWVKQAPGKGLKWMGWINTYSGVPTYADDFKGRFAFSLETSASTAYLQINNLKNEDTATYFCAVPYEYDGAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 479 - protein, 441 aa, synthetic construct
  REGION 1..441: note = HC-44
EVQLQQSGPELVKPGASVKISCKASGYTFTDYYMNWVKQSHGKSLEWIGDINPNNGGTNYNQKFKGKATLTVDKSSSTAYMELRSLTSEDSAVYYCTSGYGFPYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 480 - protein, 443 aa, synthetic construct
  REGION 1..443: note = HC-45
QIQLVQSGPELKKPGETVKISCKASGYTFTTYGMGWVKQAPGKGLKWMGWINTYSGVPTYADDFKGRFAFSLETSTSTAYLQINNLKNEDMATYFCATPYEYDGAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 481 - protein, 438 aa, synthetic construct
  REGION 1..438: note = HC-46
QVQLQQSGPELVKPGDSVKISCKAAGYTFSDYYINWVKQRPGQGLEWIGWIYPVSVNTKYNEKFKGKATLTVDTSSSTAYMQLSSLTSEDSGVYFCAYLDYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 482 - protein, 446 aa, synthetic construct
  REGION 1..446: note = HC-47
QVQLKQSGPGLVQPSQSLSITCTVSGFSLTSYGVHWVRQAPGKGLEWLGVIWSGGSTDYNAAFMSRLSISKDNSKSQVFFKMNSLQADDTAIYYCAKSPDGYDVAWFGYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 483 - protein, 218 aa, synthetic construct
  REGION 1..218: note = LC-28
DIVLTQSPASLGVSLGQRATISCRASKSVSTSGYSYMHWYQQKPGQPPKLLIYLASILESGVPARFSGSGSGTDFTLNIHPVEEEDAAIYYCQHSRELPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 484 - protein, 218 aa, synthetic construct
  REGION 1..218: note = LC-29
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 485 - protein, 219 aa, synthetic construct
  REGION 1..219: note = LC-30
DVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPQTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 486 - protein, 219 aa, synthetic construct
  REGION 1..219: note = LC-31
DIVITQDELSNPVTSGESVSISCRSSKSLLYKDGKTYLNWFLQRPGQSPQLLIYLMSTHASGVSDRFSGSGSGTDFTLEISRVKAEDVGVYYCQQLVDYPLTFGAGTKLELKRADVAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 487 - protein, 219 aa, synthetic construct
  REGION 1..219: note = LC-32
DIVMTQAAFSNPVTLGTSASISCRSSKSLLHSDGITYLYWYLQRPGQSPQLLIYRMSNLASGVPDRFSGSGSGTDFTLRISRVEAEDVGVYYCAQMLEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 488 - protein, 219 aa, synthetic construct
  REGION 1..219: note = LC-33
DIVMTQTPLSLSVTPGQPASISCKSSKSLLHSDGITYLYWYLQKPGQSPQLLIYRMSNLASGVPDRFSGSGSGTDFTLKISRVEAEDVGVYYCAQMLEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 489 - protein, 219 aa, synthetic construct
  REGION 1..219: note = LC-34
DIVMTQSPLSLPVTPGEPASISCRSSKSLLHSDGITYLYWYLQKPGQSPQLLIYRMSNLASGVPDRFSGSGSGTDFTLKISRVEAEDVGVYYCAQMLEFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 490 - protein, 219 aa, synthetic construct
  REGION 1..219: note = LC-35
DVVMTQTPLTLSVTIGQPASISCKSSQSLLYTDGKTYLSWFLQRPGQSPKRLIYLVSKLDSGVPDRFSGSGSGTDFTLKISRVEAEDLGVYYCLQSTHFPLTFGAGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 491 - protein, 214 aa, synthetic construct
  REGION 1..214: note = LC-36
SIVMTQTPKFLLVSAGDRVTITCKASQSVSNDVAWYQQKPGQSPKLLIYYASNRYTGVPDRFTGSGYGTDFTFTISTVQAEDLAVYFCQQDYTSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 214 aa, synthetic construct
  REGION 1..214: note = LC-37
DIQMTQTTSSLSASLGDRVTISCSASQGISNYLNWYQQKPDGTVKLLIYYTSSLHSGVPSRFSGSGSGTDYSLTISNLEPEDIATYYCQQYSELPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 493 - protein, 214 aa, synthetic construct
  REGION 1..214: note = LC-38
DIVMTQSHKFMSTSVGDRVSITCKASQDVSTAVAWYQQKPGQSPKLLIYWASTRHTGVPDRFTGSGSGTDYTLTISSVQAEDLALYYCQQHYTTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 214 aa, synthetic construct
  REGION 1..214: note = LC-39
DIVMTQSQKFMSTSVGERVSITCKASQNVGTNVAWYQQKAGQSLELLIYGASNRHTGVPDRFTGSGSGTDFTLTITNVQSEDMTNYFCEQYSNFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 495 - protein, 219 aa, synthetic construct
  REGION 1..219: note = LC-40
DVVMTQTPLTLSVTIGQPASISCKSSQSLFHSDGKTYLNWLLQRPGQSPKRLIYLVSKLDSGVPDRFTGSGSGTDFTPKISRVEDEDLGVYYCWQGTHFPLTFGAGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 496 - protein, 214 aa, synthetic construct
  REGION 1..214: note = LC-41
DIQMNQSPSSLSASLGDTISITCRASQNINIWLSWYQQKPGNIPQLLIYKASNLHTGVPSRFSGSGSGTDFTLTISSLQPEDIATYYCLQGQSYPLTFGAGTKLVMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 497 - protein, 217 aa, synthetic construct
  REGION 1..217: note = LC-42
DVVMTQTPLTLSVTIGQPASISCKSSQSLLHSDGKTYLNWLLQRPGQPPKRLMYLVSTLDSGVPDRFTGSGSGTDFTLKISRVEAEDLGVYYCWQGTHFPLTFGAGTKPERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 498 - protein, 217 aa, synthetic construct
  REGION 1..217: note = LC-43
DIVMTQAAFSNPVTLGTSASISCRSSKSLLHSDGITYFYWYLQRPGQSPQLLIYRMSNLASGVPDRFSGSGSGTDFTLRISRVEAEDVGVYYCAQMLEFPLTFGAGTKLERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 499 - protein, 214 aa, synthetic construct
  REGION 1..214: note = LC-44
DIVMTQSHKFMSTSVGDRVSITCKASQDVRTAVAWYQQKPGQSPKLLIYWASTRHTGVPDRFTGSGSGTDFTLTLSNVQSEDLADYFCQQYSSYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 500 - DNA, 354 bp, synthetic construct
  misc_feature 1..354: note = 846.2B11 VH nucleic acid
  source 1..354: mol_type = other DNA
caggtccagctacagcagtctggacctgagctggtgaggcctggggctttagtgaagatttcctgcaaggcttctggttacaccttcacaagctacgatataaactgggtgaagcagaggcctggacagggacttgagtggattggatggatttatcctggagatggtagtactaaatataatgagaaattcaagggcaaggccacactgactgcagacaaatcctccagcacagcctacatgcagctcagcagcctgacttctgagaactctgcagtctatttctgtgcaagagaggcggcttctaatgctatggactactggggtcaaggaacctcagtcaccgtctcctca

SEQ ID NO: 501 - DNA, 366 bp, synthetic construct
  misc_feature 1..366: note = 846.4D5 VH nucleic acid
  source 1..366: mol_type = other DNA
gaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccaggcactacggctttccctactggtacttcgatgtctggggcgcagggaccacggtcaccgtctcctca

SEQ ID NO: 502 - DNA, 342 bp, synthetic construct
  misc_feature 1..342: note = 847.14H4 VH nucleic acid
  source 1..342: mol_type = other DNA
cagatccagttggtgcagtctggacctgaactgacgaagcctggagagacagtcaagatctcctgtaaggcttctggctataccttcacagactatggaatgaactgggtgaggcaggctccaggagagactttaaagtggatgggctggataaacacctacactggagagccaacatatgctgatgacttcaaggggcggtttgccttctctttggaatcctctgccagcactgcctatttgcagatcaacaacctcaaaaatgaggacgtggctacatatttctgtgcaagataccctatggactactggggtcaaggaacctcagtcaccgtctcctca

SEQ ID NO: 503 - DNA, 354 bp, synthetic construct
  misc_feature 1..354: note = 846T.1H2 VH nucleic acid
  source 1..354: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacatatgctgaagagttcaagggacggtttgccttctctttggaaacctctgccagcactgcctatttgcagatcaacaacctcaaaaatgaggacacggctacatatttctgtgcaaccggggggggtaactgggactttgactactggggccaaggcaccactctcacagtctcctca

SEQ ID NO: 504 - DNA, 348 bp, synthetic construct
  misc_feature 1..348: note = mIMT001 VH nucleic acid
  source 1..348: mol_type = other DNA
cagatccaacttgtgcagtcaggtccagaattgaagaagcctggtgagaccgtcaagatttcctgtaaggcctctggttatacttttaccaactatggcatgaattgggtcaagcaggcacctgggaagggtttgaaatggatggggtggataaataccaacacaggggaaccaacttacgttgaagagtttaagggtcgttttgcattcagtcttgagacttctgcaagcacagcttacctccaaattaacaatctcaaaaatgaagatactgctacatatttctgtgctccttatgataattttttcgcctactggggacagggaactctggtcaccgtctcagca

SEQ ID NO: 505 - DNA, 348 bp, synthetic construct
  misc_feature 1..348: note = 4A11.2B5 VH nucleic acid
  source 1..348: mol_type = other DNA
caggtacaacttcagcagagcggtccagaacttgtgaagcccggtgctagtgtcaaaatatcttgcaaagcaagtggttattccttcaccaactattacatacactgggtaaaacaacgtccagggcaaggactcgaatggattggttggatctaccccggcagcggcaacactaactacaacgagaagtttaaaggcaaagctactctcacagcagacacttctagcagtacaacaaatatgcagttgtcctctctgacttccgaagacagcgccgtctattactgttctactgcacccggaggttttgacgtctggggttccggcaccacagttaccgttagttcc

SEQ ID NO: 506 - DNA, 348 bp, synthetic construct
  misc_feature 1..348: note = 4A11.H1L1 VH nucleic acid
  source 1..348: mol_type = other DNA
caggtgcagctggtgcaatcaggagccgaagttaagaagcctggcgccagcgtcaaggtctcatgtaaagcctccggttacagtttcaccaactactatatgcactgggttaggcaagcaccaggccaggggcttgagtggatgggatggatctatccagggagcggtaacaccaattacaatgagaagtttcaagggcgcgtaaccatgacagcagataccagtataagcaccgcctatatggaactctctcggttgagatccgatgataccgctgtttattactgctctaccgcacctggtggtttcgacgtttggggccaaggtactacagtgaccgtatcatca

SEQ ID NO: 507 - DNA, 348 bp, synthetic construct
  misc_feature 1..348: note = 4A11.H4L2 VH nucleic acid
  source 1..348: mol_type = other DNA
caggtgcaactggtccagtctggggccgaggtcaaaaagcctggagcctccgtcaaagtgtcatgtaaagcctcagggtatagtttcaccaactactatatacattgggtcagacaagccccaggccagcgtcttgagtggatgggatggatttatcccggatcagggaatacaaactataacgagaaatttcaaggcagagttactctcactgccgatacctctgcaagtactacctatatggagctctccagtctcagaagcgaagacacagcagtatattactgtagcaccgccccaggcgggtttgacgtgtggggccaaggcactactgtaaccgttagctca

SEQ ID NO: 508 - DNA, 357 bp, synthetic construct
  misc_feature 1..357: note = 4G2.2G6 VH nucleic acid
  source 1..357: mol_type = other DNA
caaatacagctcgtgcagtctggtcccgatttgaagaagcctggagaaaccgtaaagataagctgtaaggctagtggttacactttcacaacatacgttatgtcatgggttaaacaggctcctggaaaagaccttaaatggatgggatggattaacacccatagcggtgtcccaacttacgctgacgattttaagggacgatttgacttttctctcgaaacttctgccaacacagcttttttgcagatcaacaatctcaagaatgaagacaccgcaacctatttctgcactcgagatgggaacgacggggatgcaatggataactggggacaaggcacatcagttaccgtgagctct

SEQ ID NO: 509 - DNA, 360 bp, synthetic construct
  misc_feature 1..360: note = 6B3.2D3 VH nucleic acid
  source 1..360: mol_type = other DNA
caggtgcagctgaagcagtcaggacctggcctagtgcagccctcacagagcctgtccatcacctgcacagtctctggtttctcattaactagttatggtgtacactgggttcgccagcctccaggaaagggtctggagtggctgggagtgatatggagtggtggaagcacagactataatgctgctttcatatccagactgagcatcagtaaggacaactccaagagccaagttttctttaaaatgaacagtctgcaagctgatgacactgccatatactactgtgccaaagggccttatgattacgacttaggctggtttgcttactggggccaagggactctggtcactgtctctgca

SEQ ID NO: 510 - DNA, 348 bp, synthetic construct
  misc_feature 1..348: note = 6H6.2D6 VH nucleic acid
  source 1..348: mol_type = other DNA
cagatccagctggtgcagtccggccccgagctgaagaagcccggcgagactgtgaagatctcctgcaaggcctccggctacaccttcaccacctacggcatgtcctgggtgaagcaggcccccggcaagggcctgaagtggatggcctggatcaacacctactccggcgtgcccacctacgccgacgacttcaagggcaggttcgccttctccctggagacttccgcctccaccgcctacctgcagatcaacaacctgaccaacgaggacaccgccacctacttctgcgccaggggcccctacgccatggactactggggccagggcacctccgtgaccgtgtcctcc

SEQ ID NO: 511 - DNA, 345 bp, synthetic construct
  misc_feature 1..345: note = 9H2.2H10 VH nucleic acid
  source 1..345: mol_type = other DNA
caggtccagctgcagcagcctggggctgaactggtggggcctgggtcttcagtgaagctgtcctgcaaggcttctggctacaccttcaccaactactggatacattgggtgaagcagaggcctctacaaggccttgaatggattggtaacattgacccttctgatagtgaaactcactacaatcaaaagttcaaggacaaggccacattgactgtagacaaatcctccagcacagcctacatgcaactcagcagcctgacatctgaggactctgcggtctattactgtgcaagacatggttactacgactactggggccaaggcaccactctcacagtctcctca

SEQ ID NO: 512 - DNA, 357 bp, synthetic construct
  misc_feature 1..357: note = 13G4.2F8 VH nucleic acid
  source 1..357: mol_type = other DNA
caggtccagctgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaacagtggtagtactgactacaatgagaagttcaagaacaaggccacactgaatgtagacaaatcctccagtacagcctacatacaactcagcagcctgacatctgaggactctgcggtctattactgtacaagatgggggatttattactacgcgagggactactggggccaaggcaccactctcacagtctcctca

SEQ ID NO: 513 - DNA, 351 bp, synthetic construct
  misc_feature 1..351: note = 13H12.2F8 VH nucleic acid
  source 1..351: mol_type = other DNA
cagatccagctggtccaatcaggacccgaactgaaaaaaccaggcgagacagtcaaaatcagctgtaaggcctcaggctacactttcacaacctacgggatgtcatgggtaaagcaagctcctggcaaggggctgaaatggatgggttggatcaacacatactctggagtgcccacctacgctgacgactttaagggtagattcgcatttagcctggaaacaagtgccagtacagcctacctccagataaacaacttgaaaaacgaggataccgcaacctatttttgcgccgtcccctacgagtacgacggtgcctattggggtcagggtacactcgtaacagtttccgcc

SEQ ID NO: 514 - DNA, 345 bp, synthetic construct
  misc_feature 1..345: note = 15G7.2A7 VH nucleic acid
  source 1..345: mol_type = other DNA
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactaactacaaccagaagttcaagggcaaggccacattgactgtagacaagtcctccagcacagcctacatggagctccgcagcctgacatctgaggactctgcagtctattactgcacctcaggctacgggtttccttactggggccaggggactctggtcactgtctctgca

SEQ ID NO: 515 - DNA, 351 bp, synthetic construct
  misc_feature 1..351: note = 19D9.2E5 VH nucleic acid
  source 1..351: mol_type = other DNA
cagatccagttggtacagtctggacctgagctgaagaagcctggagagacagtcaagatctcctgcaaggcttctggatataccttcacaacctatggaatgggctgggtgaaacaggctccaggaaagggtttgaagtggatgggctggataaacacctactctggcgtgccaacatatgctgatgacttcaagggacggtttgccttctctttggaaacctctaccagcactgcctatttgcagatcaacaacctcaaaaatgaggacatggctacatatttctgtgcaaccccttatgaatacgacggggcttactggggccaagggactctggtcactgtctctgca

SEQ ID NO: 516 - DNA, 336 bp, synthetic construct
  misc_feature 1..336: note = 23B10.2B12 VH nucleic acid
  source 1..336: mol_type = other DNA
caggtccagctgcagcagtctggacctgaactggtgaagcctggggattctgtgaagatatcctgcaaggctgctggctacaccttcagtgactattatataaactgggtgaagcagaggcctggacagggacttgagtggattggatggatttatcctgtaagcgttaatactaagtacaatgagaagttcaagggcaaggccacattgactgtagacacatcctccagcacagcctacatgcagctcagcagcctgacctctgaggactctggggtctatttctgtgcctaccttgattattggggccaagggactctggtcactgtctctgca

SEQ ID NO: 517 - DNA, 360 bp, synthetic construct
  misc_feature 1..360: note = 24D12.2H9 VH nucleic acid
  source 1..360: mol_type = other DNA
caggtgcagctgaagcagtcaggacctggcctagtgcagccctcacagagcctgtccatcacctgcacagtctctggtttctcattaactagctatggtgtacactgggttcgccaggctccaggaaagggtctggaatggctgggagtgatatggagtggtggaagcacagactataatgctgctttcatgtccagactgagcatcagcaaggacaactccaagagccaagttttctttaaaatgaacagtctgcaagctgatgacactgccatatactactgtgccaaaagccctgatggttacgacgtcgcctggtttggttactggggccaagggactctggtcactgtctctgca

SEQ ID NO: 518 - DNA, 333 bp, synthetic construct
  misc_feature 1..333: note = 846.2B11 VL nucleic acid
  source 1..333: mol_type = other DNA
gacattgtgctgacacagtctcctgcttccttaggtgtttctctggggcagagggccaccatctcatgcagggccagcaaaagtgtcagtacatctggctatagttatatgcactggtaccaacagaaaccaggacagccacccaaactcctcatctatcttgcatccatcctagaatctggggtccctgccaggttcagtggcagtgggtctgggacagacttcaccctcaacatccatcctgtggaggaggaggatgctgcaatctattactgtcagcacagtagggagcttcctctcacgttcggtgctgggaccaagctggagctgaaa

SEQ ID NO: 519 - DNA, 333 bp, synthetic construct
  misc_feature 1..333: note = 846.4D5 VL nucleic acid
  source 1..333: mol_type = other DNA
aacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaa

SEQ ID NO: 520 - DNA, 327 bp, synthetic construct; identical to SEQ ID NO: 315
  misc_feature 1..327: note = 847.14H4 VL nucleic acid
  source 1..327: mol_type = other DNA

SEQ ID NO: 521 - DNA, 336 bp, synthetic construct
  misc_feature 1..336: note = 846T.1H2 VL nucleic acid
  source 1..336: mol_type = other DNA
gatgttgtgatgacccagactccactcactttgtcggttaccattggacaaccagcctctatctcttgcaagtcaagtcagagtctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacattttcctcagacgttcggtggaggcaccaagctggaaataaaa

SEQ ID NO: 522 - DNA, 336 bp, synthetic construct
  misc_feature 1..336: note = mIMT001 VL nucleic acid
  source 1..336: mol_type = other DNA
gatattgttattactcaggatgagcttagtaatcctgtcactagcggggaaagtgttagtatcagttgtcgttcatccaagtcactcctctacaaagacggcaagacttatctcaactggtttcttcaacgacccggtcaatctccacagctcctgatttacctgatgtcaactcacgcttctggtgttagcgaccgtttctccggaagtggctccgggacagacttcactcttgaaatctcacgtgtaaaagcagaggacgtaggtgtctattattgtcaacagcttgtggactaccccttgacctttggcgctggcacaaagctggaactgaag

SEQ ID NO: 523 - DNA, 336 bp, synthetic construct
  misc_feature 1..336: note = 4A11.2B5 VL nucleic acid
  source 1..336: mol_type = other DNA
gacattgtgatgactcaggcagccttttccaaccctgtaacacttggaactagtgcctccatctcatgccgttccagcaagagtcttttgcacagcgatgggataacctatctctattggtatctgcaacgacccggtcagtcaccccaacttcttatctatcgaatgtctaacttggcctccggcgtgcccgaccgcttttccgggagtgggagcggtacagatttcaccttgcgaatttcccgggttgaggcagaggacgtaggtgtctactattgtgctcagatgcttgagtttcctctcactttcggagccggcaccaaactggaactgaaa

SEQ ID NO: 524 - DNA, 336 bp, synthetic construct; identical to SEQ ID NO: 290
  misc_feature 1..336: note = 4A11.H1L1 VL nucleic acid
  source 1..336: mol_type = other DNA

SEQ ID NO: 525 - DNA, 336 bp, synthetic construct
  misc_feature 1..336: note = 4A11.H4L2 VL nucleic acid
  source 1..336: mol_type = other DNA
gatattgtaatgacccaaagccccctgtccttgcctgttaccccaggtgagcctgccagcatatcttgccgatccagtaaatctctgcttcatagcgatggcatcacctatttgtattggtatttgcagaagcccggccagagcccccagttgctgatctacagaatgtccaaccttgctagtggagtgcccgaccggtttagcggttcaggctcagggactgatttcactcttaaaatcagtagggtagaggccgaagacgttggtgtatattattgtgctcagatgttggagtttccactcacatttggacaaggtacaaagctggaaattaag

SEQ ID NO: 526 - DNA, 336 bp, synthetic construct
  misc_feature 1..336: note = 4G2.2G6 VL nucleic acid
  source 1..336: mol_type = other DNA
gatgtcgtaatgactcaaacccccctgacccttagtgttactataggccagcccgcaagcatctcctgcaaatcctcccagtctcttctttatactgatggcaaaacctacttgtcctggtttttgcagcgcccaggtcaaagtcctaaacgtcttatttacttggtctctaaactcgattccggcgtgcctgaccggttctccggtagcggtagcggtaccgatttcactttgaagattagccgagttgaggcagaagacctcggggtctactactgccttcagagtacacacttcccacttacatttggcgcaggaaccaagttggaggttaaa

SEQ ID NO: 527 - DNA, 321 bp, synthetic construct
  misc_feature 1..321: note = 6B3.2D3 VL nucleic acid
  source 1..321: mol_type = other DNA
agtattgtgatgacccagactcccaaattcctgcttgtatcagcaggagacagggttaccataacctgcaaggccagtcagagtgtgagtaatgatgtagcttggtaccaacagaagccagggcagtctcctaaattgttgatatattatgcatccaatcgctacactggagtccctgatcgcttcactggcagtggatatgggacggatttcactttcaccatcagcactgtgcaggctgaagacctggcagtttatttctgtcagcaggattatacctctccgtacacgttcggaggggggaccaagctggaaataaaa

SEQ ID NO: 528 - DNA, 321 bp, synthetic construct
  misc_feature 1..321: note = 6H6.2D6 VL nucleic acid
  source 1..321: mol_type = other DNA
gacatccagatgacccagaccacctcctccctgtccgcctccctgggcgacagggtgaccatctcctgctccgcctcccagggcatctccaactacctgaactggtaccagcagaagcccgacggcaccgtgaagctgctgatctactacacctcctccctgcactccggcgtgccctccaggttctccggctccggctccggcaccgactactccctgaccatctccaacctggagcccgaggacatcgccacctactactgccagcagtactccgagctgccctacaccttcggctccggcaccaagctggagatcaag

SEQ ID NO: 529 - DNA, 321 bp, synthetic construct
  misc_feature 1..321: note = 9H2.2H10 VL nucleic acid
  source 1..321: mol_type = other DNA
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattataccactccgctcacgttcggtgctgggaccaagctggagctgaaa

SEQ ID NO: 530 - DNA, 321 bp, synthetic construct
  misc_feature 1..321: note = 13G4.2F8 VL nucleic acid
  source 1..321: mol_type = other DNA
gacattgtgatgacccagtctcaaaaattcatgtccacatcagtaggagagagggtcagcatcacctgcaaggccagtcagaatgtaggtactaatgtagcctggtatcagcagaaagcagggcagtctcttgaactgctgatctatggggcatccaaccggcacactggagtccctgatcgcttcacaggcagtggatctgggacagatttcactctcaccatcaccaatgtgcagtctgaagacatgacaaattatttctgtgagcaatatagcaactttcctctcacgttcggtgctgggaccaagctggagctgaaa

SEQ ID NO: 531 - DNA, 336 bp, synthetic construct
  misc_feature 1..336: note = 13H12.2F8 VL nucleic acid
  source 1..336: mol_type = other DNA
gacgttgttatgacacaaacaccacttacactttctgtaactatcggtcagcctgcctcaatttcatgtaagtcttctcagtccctgtttcatagcgacgggaagacttacctgaattggcttctccaacgaccaggccaatcacccaagcgccttatttatttggtcagtaaattggacagtggagttcccgatagatttaccgggagcggttctgggaccgatttcaccccaaagatctccagggtagaagacgaagatctcggtgtgtactattgctggcaggggacacatttccctctgacattcggtgctggaaccaaacttgagatgaaa

SEQ ID NO: 532 - DNA, 321 bp, synthetic construct
  misc_feature 1..321: note = 15G7.2A7 VL nucleic acid
  source 1..321: mol_type = other DNA
gacatccagatgaaccagtctccatccagtctgtctgcatccctcggagacacaatttccatcacttgccgtgccagtcagaacattaatatttggttaagctggtaccagcagaaaccaggaaatattcctcaactattgatctataaggcttccaacttgcacacaggcgtcccctcaaggtttagtggcagtggatctggaacagatttcacattaaccatcagcagtctgcagcctgaagacattgccacttactactgtctacagggtcaaagttatcctctcacgttcggtgctgggaccaagctggtgatgaaa

SEQ ID NO: 533 - DNA, 330 bp, synthetic construct
  misc_feature 1..330: note = 19D9.2E5 VL nucleic acid
  source 1..330: mol_type = other DNA
gatgttgtgatgacccagactccactcactttgtcggttaccattggacaaccagcctccatctcttgtaagtcaagtcagagcctcttacatagtgatggaaagacatatttgaattggttgttacagaggccaggccagcctccaaagcgcctaatgtatctggtgtctacactggactctggagtccctgacaggttcactggcagtggatcagggacagatttcacactgaaaatcagcagagtggaggctgaggatttgggagtttattattgctggcaaggtacacattttcctctcacgttcggtgctgggaccaagccggag

SEQ ID NO: 534 - DNA, 330 bp, synthetic construct
  misc_feature 1..330: note = 23B10.2B12 VL nucleic acid
  source 1..330: mol_type = other DNA
gatattgtgatgacgcaggctgcattctccaatccagtcactcttggaacgtcagcttccatctcctgcaggtctagtaagagtctcctacatagtgatggcatcacttatttctattggtatctgcagaggccaggccagtctcctcagctcctgatatatcggatgtccaaccttgcctcaggagtcccagacaggttcagtggcagtgggtcaggaactgatttcacactgagaatcagcagagtggaggctgaggatgtgggtgtttattactgtgctcaaatgctagaattcccgctcacgttcggtgctgggaccaagctggag

SEQ ID NO: 535 - DNA, 321 bp, synthetic construct
  misc_feature 1..321: note = 24D12.2H9 VL nucleic acid
  source 1..321: mol_type = other DNA
gacattgtgatgacccagtctcacaaattcatgtccacatcagtaggagacagggtcagcatcacctgcaaggccagtcaggatgtgcgtactgctgtagcctggtatcaacagaaaccagggcaatctcctaaactactgatttactgggcatccacccggcacactggagtccctgatcgcttcacaggcagtggatctgggacagatttcactctcacccttagcaatgtgcagtctgaagacttggcagattatttctgtcagcaatatagtagttatccgtggacgttcggtggaggcaccaagctggaaatcaaa

SEQ ID NO: 536 - DNA, 1338 bp, synthetic construct
  misc_feature 1..1338: note = 846.2B11 HC nucleic acid
  source 1..1338: mol_type = other DNA
caggtccagctacagcagtctggacctgagctggtgaggcctggggctttagtgaagatttcctgcaaggcttctggttacaccttcacaagctacgatataaactgggtgaagcagaggcctggacagggacttgagtggattggatggatttatcctggagatggtagtactaaatataatgagaaattcaagggcaaggccacactgactgcagacaaatcctccagcacagcctacatgcagctcagcagcctgacttctgagaactctgcagtctatttctgtgcaagagaggcggcttctaatgctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 537 - DNA, 1350 bp, synthetic construct
  misc_feature 1..1350: note = 846.4D5 HC nucleic acid
  source 1..1350: mol_type = other DNA
gaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccaggcactacggctttccctactggtacttcgatgtctggggcgcagggaccacggtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 538 - DNA, 1326 bp, synthetic construct
  misc_feature 1..1326: note = 847.14H4 HC nucleic acid
  source 1..1326: mol_type = other DNA
cagatccagttggtgcagtctggacctgaactgacgaagcctggagagacagtcaagatctcctgtaaggcttctggctataccttcacagactatggaatgaactgggtgaggcaggctccaggagagactttaaagtggatgggctggataaacacctacactggagagccaacatatgctgatgacttcaaggggcggtttgccttctctttggaatcctctgccagcactgcctatttgcagatcaacaacctcaaaaatgaggacgtggctacatatttctgtgcaagataccctatggactactggggtcaaggaacctcagtcaccgtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 539 - DNA, 1338 bp, synthetic construct
  misc_feature 1..1338: note = 846T.1H2 HC nucleic acid
  source 1..1338: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacatatgctgaagagttcaagggacggtttgccttctctttggaaacctctgccagcactgcctatttgcagatcaacaacctcaaaaatgaggacacggctacatatttctgtgcaaccggggggggtaactgggactttgactactggggccaaggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 540 - DNA, 1344 bp, synthetic construct
  misc_feature 1..1344: note = mIMT001 HC nucleic acid
  source 1..1344: mol_type = other DNA
cagatccaacttgtgcagtcaggtccagaattgaagaagcctggtgagaccgtcaagatttcctgtaaggcctctggttatacttttaccaactatggcatgaattgggtcaagcaggcacctgggaagggtttgaaatggatggggtggataaataccaacacaggggaaccaacttacgttgaagagtttaagggtcgttttgcattcagtcttgagacttctgcaagcacagcttacctccaaattaacaatctcaaaaatgaagatactgctacatatttctgtgctccttatgataattttttcgcctactggggacagggaactctggtcaccgtctcagcagctaagactactcctcccagtgtctaccccctggcccccgtgtgcggcgacacaacaggctcctccgtgacactgggctgcctggtgaaaggctacttccccgagcccgtgacactgacatggaactccggctccctgtcctccggcgtgcacacattccccgccgtgctgcagtccgacctgtacacactgtcctcctccgtgacagtgacatcctccacatggccctcccagtccatcacatgcaacgtggcccaccccgcctcctccacaaaagtggacaaaaaaatcgagcccaggggccccacaatcaaaccctgccccccctgcaaatgccccgcccccaacgccgccggcggcccctccgtgttcatcttcccccccaaaatcaaagacgtgctgatgatctccctgtcccccatcgtgacatgcgtggtggtggacgtgtccgaggacgaccccgacgtgcagatctcctggttcgtgaacaacgtggaggtgcacacagcccagacacagacacacagggaggactacaactccacactgagggtggtgtccgccctgcccatccagcaccaggactggatgtccggcaaagagttcaaatgcaaagtgaacaacaaagacctgggcgcccccatcgagaggacaatctccaaacccaaaggctccgtgagggccccccaggtgtacgtgctgcccccccccgaggaggagatgacaaaaaaacaggtgacactgacatgcatggtgacagacttcatgcccgaggacatctacgtggagtggacaaacaacggcaaaacagagctgaactacaaaaacacagagcccgtgctggactccgacggctcctacttcatgtactccaaactgagggtggagaaaaaaaactgggtggagaggaactcctactcctgctccgtggtgcacgagggcctgcacaaccaccacacaacaaaatccttctccaggacacccggcaaatagtaa

SEQ ID NO: 541 - DNA, 1332 bp, synthetic construct
  misc_feature 1..1332: note = 4A11.2B5 HC nucleic acid
  source 1..1332: mol_type = other DNA
caggtacaacttcagcagagcggtccagaacttgtgaagcccggtgctagtgtcaaaatatcttgcaaagcaagtggttattccttcaccaactattacatacactgggtaaaacaacgtccagggcaaggactcgaatggattggttggatctaccccggcagcggcaacactaactacaacgagaagtttaaaggcaaagctactctcacagcagacacttctagcagtacaacaaatatgcagttgtcctctctgacttccgaagacagcgccgtctattactgttctactgcacccggaggttttgacgtctggggttccggcaccacagttaccgttagttccgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 542 - DNA, 1332 bp, synthetic construct
  misc_feature 1..1332: note = 4A11.H1L1 HC nucleic acid
  source 1..1332: mol_type = other DNA
caggtgcagctggtgcaatcaggagccgaagttaagaagcctggcgccagcgtcaaggtctcatgtaaagcctccggttacagtttcaccaactactatatgcactgggttaggcaagcaccaggccaggggcttgagtggatgggatggatctatccagggagcggtaacaccaattacaatgagaagtttcaagggcgcgtaaccatgacagcagataccagtataagcaccgcctatatggaactctctcggttgagatccgatgataccgctgtttattactgctctaccgcacctggtggtttcgacgtttggggccaaggtactacagtgaccgtatcatcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 543 - DNA, 1332 bp, synthetic construct
  misc_feature 1..1332: note = 4A11.H4L2 HC nucleic acid
  source 1..1332: mol_type = other DNA
caggtgcaactggtccagtctggggccgaggtcaaaaagcctggagcctccgtcaaagtgtcatgtaaagcctcagggtatagtttcaccaactactatatacattgggtcagacaagccccaggccagcgtcttgagtggatgggatggatttatcccggatcagggaatacaaactataacgagaaatttcaaggcagagttactctcactgccgatacctctgcaagtactacctatatggagctctccagtctcagaagcgaagacacagcagtatattactgtagcaccgccccaggcgggtttgacgtgtggggccaaggcactactgtaaccgttagctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 544 - DNA, 1341 bp, synthetic construct
  misc_feature 1..1341: note = 4G2.2G6 HC nucleic acid
  source 1..1341: mol_type = other DNA
caaatacagctcgtgcagtctggtcccgatttgaagaagcctggagaaaccgtaaagataagctgtaaggctagtggttacactttcacaacatacgttatgtcatgggttaaacaggctcctggaaaagaccttaaatggatgggatggattaacacccatagcggtgtcccaacttacgctgacgattttaagggacgatttgacttttctctcgaaacttctgccaacacagcttttttgcagatcaacaatctcaagaatgaagacaccgcaacctatttctgcactcgagatgggaacgacggggatgcaatggataactggggacaaggcacatcagttaccgtgagctctgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 545 - DNA, 1344 bp, synthetic construct
  misc_feature 1..1344: note = 6B3.2D3 HC nucleic acid
  source 1..1344: mol_type = other DNA
caggtgcagctgaagcagtcaggacctggcctagtgcagccctcacagagcctgtccatcacctgcacagtctctggtttctcattaactagttatggtgtacactgggttcgccagcctccaggaaagggtctggagtggctgggagtgatatggagtggtggaagcacagactataatgctgctttcatatccagactgagcatcagtaaggacaactccaagagccaagttttctttaaaatgaacagtctgcaagctgatgacactgccatatactactgtgccaaagggccttatgattacgacttaggctggtttgcttactggggccaagggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 546 - DNA, 1332 bp, synthetic construct
  misc_feature 1..1332: note = 6H6.2D6 HC nucleic acid
  source 1..1332: mol_type = other DNA
cagatccagctggtgcagtccggccccgagctgaagaagcccggcgagactgtgaagatctcctgcaaggcctccggctacaccttcaccacctacggcatgtcctgggtgaagcaggcccccggcaagggcctgaagtggatggcctggatcaacacctactccggcgtgcccacctacgccgacgacttcaagggcaggttcgccttctccctggagacttccgcctccaccgcctacctgcagatcaacaacctgaccaacgaggacaccgccacctacttctgcgccaggggcccctacgccatggactactggggccagggcacctccgtgaccgtgtcctccgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 547 - DNA, 1329 bp, synthetic construct
  misc_feature 1..1329: note = 9H2.2H10 HC nucleic acid
  source 1..1329: mol_type = other DNA
caggtccagctgcagcagcctggggctgaactggtggggcctgggtcttcagtgaagctgtcctgcaaggcttctggctacaccttcaccaactactggatacattgggtgaagcagaggcctctacaaggccttgaatggattggtaacattgacccttctgatagtgaaactcactacaatcaaaagttcaaggacaaggccacattgactgtagacaaatcctccagcacagcctacatgcaactcagcagcctgacatctgaggactctgcggtctattactgtgcaagacatggttactacgactactggggccaaggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 548 - DNA, 1341 bp, synthetic construct
  misc_feature 1..1341: note = 13G4.2F8 HC nucleic acid
  source 1..1341: mol_type = other DNA
caggtccagctgcagcagcctggggctgagctggtaaagcctggggcttcagtgaagttgtcctgcaaggcttctggctacactttcaccagctactggatgcactgggtgaagcagaggcctggacaaggccttgagtggattggaatgattcatcctaacagtggtagtactgactacaatgagaagttcaagaacaaggccacactgaatgtagacaaatcctccagtacagcctacatacaactcagcagcctgacatctgaggactctgcggtctattactgtacaagatgggggatttattactacgcgagggactactggggccaaggcaccactctcacagtctcctcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 549 - DNA, 1335 bp, synthetic construct
  misc_feature 1..1335: note = 13H12.2F8 HC nucleic acid
  source 1..1335: mol_type = other DNA
cagatccagctggtccaatcaggacccgaactgaaaaaaccaggcgagacagtcaaaatcagctgtaaggcctcaggctacactttcacaacctacgggatgtcatgggtaaagcaagctcctggcaaggggctgaaatggatgggttggatcaacacatactctggagtgcccacctacgctgacgactttaagggtagattcgcatttagcctggaaacaagtgccagtacagcctacctccagataaacaacttgaaaaacgaggataccgcaacctatttttgcgccgtcccctacgagtacgacggtgcctattggggtcagggtacactcgtaacagtttccgccgctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 550 - DNA, 1329 bp, synthetic construct
  misc_feature 1..1329: note = 15G7.2A7 HC nucleic acid
  source 1..1329: mol_type = other DNA
gaggtccagctgcaacaatctggacctgagctggtgaagcctggggcttcagtgaagatatcctgtaaggcttctggatacacgttcactgactactacatgaactgggtgaagcagagccatggaaagagccttgagtggattggagatattaatcctaacaatggtggtactaactacaaccagaagttcaagggcaaggccacattgactgtagacaagtcctccagcacagcctacatggagctccgcagcctgacatctgaggactctgcagtctattactgcacctcaggctacgggtttccttactggggccaggggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 551 - DNA, 1335 bp, synthetic construct
  misc_feature 1..1335: note = 19D9.2E5 HC nucleic acid
  source 1..1335: mol_type = other DNA
cagatccagttggtacagtctggacctgagctgaagaagcctggagagacagtcaagatctcctgcaaggcttctggatataccttcacaacctatggaatgggctgggtgaaacaggctccaggaaagggtttgaagtggatgggctggataaacacctactctggcgtgccaacatatgctgatgacttcaagggacggtttgccttctctttggaaacctctaccagcactgcctatttgcagatcaacaacctcaaaaatgaggacatggctacatatttctgtgcaaccccttatgaatacgacggggcttactggggccaagggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 552 - DNA, 1320 bp, synthetic construct
  misc_feature 1..1320: note = 23B10.2B12 HC nucleic acid
  source 1..1320: mol_type = other DNA
caggtccagctgcagcagtctggacctgaactggtgaagcctggggattctgtgaagatatcctgcaaggctgctggctacaccttcagtgactattatataaactgggtgaagcagaggcctggacagggacttgagtggattggatggatttatcctgtaagcgttaatactaagtacaatgagaagttcaagggcaaggccacattgactgtagacacatcctccagcacagcctacatgcagctcagcagcctgacctctgaggactctggggtctatttctgtgcctaccttgattattggggccaagggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 553 - DNA, 1344 bp, synthetic construct
  misc_feature 1..1344: note = 24D12.2H9 HC nucleic acid
  source 1..1344: mol_type = other DNA
caggtgcagctgaagcagtcaggacctggcctagtgcagccctcacagagcctgtccatcacctgcacagtctctggtttctcattaactagctatggtgtacactgggttcgccaggctccaggaaagggtctggaatggctgggagtgatatggagtggtggaagcacagactataatgctgctttcatgtccagactgagcatcagcaaggacaactccaagagccaagttttctttaaaatgaacagtctgcaagctgatgacactgccatatactactgtgccaaaagccctgatggttacgacgtcgcctggtttggttactggggccaagggactctggtcactgtctctgcagctagcaccaagggccccagcgtgtttcctctcgctccctgcagccggagcacatccgagagcaccgctgctctgggctgtctcgtgaaggactacttccctgaacccgtcaccgtcagctggaatagcggcgccctgacatccggcgtccacacattccccgctgtcctgcagagcagcggcctgtacagcctgagctccgtggtcaccgtgcctagcagcagcctgggaacaaagacctacacctgcaacgtggaccataagccctccaacaccaaggtggacaagcgggtggaatccaagtatggacccccctgtcctccttgccctgctcctgaatttctcggaggcccctccgtcttcctgtttccccccaagcccaaggacaccctgatgatctcccggacacccgaagtcacctgcgtcgtggtggatgtcagccaggaagatcccgaggtgcagttcaactggtacgtggacggagtggaggtgcataacgccaaaaccaagcccagggaagagcagttcaacagcacctatcgggtcgtgtccgtgctcaccgtcctgcatcaggattggctcaacggcaaggagtacaagtgcaaggtgtccaacaagggcctgccctcctccatcgagaagaccatctccaaggctaagggccaacctcgggagccccaagtgtataccctccctcccagccaggaggagatgaccaagaatcaagtgagcctgacctgcctcgtgaagggattttacccctccgacatcgctgtggaatgggaaagcaatggccaacctgagaacaactacaagaccacaccccccgtgctggactccgatggctccttcttcctgtacagcaggctgaccgtggacaaatcccggtggcaagagggaaacgtgttcagctgctccgtgatgcacgaggctctccacaaccactacacccagaagagcctctccctgagcctcggctagtaa

SEQ ID NO: 554 - DNA, 657 bp, synthetic construct
  misc_feature 1..657: note = 846.2B11 LC nucleic acid
  source 1..657: mol_type = other DNA
gacattgtgctgacacagtctcctgcttccttaggtgtttctctggggcagagggccaccatctcatgcagggccagcaaaagtgtcagtacatctggctatagttatatgcactggtaccaacagaaaccaggacagccacccaaactcctcatctatcttgcatccatcctagaatctggggtccctgccaggttcagtggcagtgggtctgggacagacttcaccctcaacatccatcctgtggaggaggaggatgctgcaatctattactgtcagcacagtagggagcttcctctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 555 - DNA, 657 bp, synthetic construct
  misc_feature 1..657: note = 846.4D5 LC nucleic acid
  source 1..657: mol_type = other DNA
aacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 556 - DNA, 651 bp, synthetic construct; identical to SEQ ID NO: 373
  misc_feature 1..651: note = 847.14H4 LC nucleic acid
  source 1..651: mol_type = other DNA

SEQ ID NO: 557 - DNA, 660 bp, synthetic construct
  misc_feature 1..660: note = 846T.1H2 LC nucleic acid
  source 1..660: mol_type = other DNA
gatgttgtgatgacccagactccactcactttgtcggttaccattggacaaccagcctctatctcttgcaagtcaagtcagagtctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacattttcctcagacgttcggtggaggcaccaagctggaaataaaaaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 558 - DNA, 663 bp, synthetic construct
  misc_feature 1..663: note = mIMT001 LC nucleic acid
  source 1..663: mol_type = other DNA
gatattgttattactcaggatgagcttagtaatcctgtcactagcggggaaagtgttagtatcagttgtcgttcatccaagtcactcctctacaaagacggcaagacttatctcaactggtttcttcaacgacccggtcaatctccacagctcctgatttacctgatgtcaactcacgcttctggtgttagcgaccgtttctccggaagtggctccgggacagacttcactcttgaaatctcacgtgtaaaagcagaggacgtaggtgtctattattgtcaacagcttgtggactaccccttgacctttggcgctggcacaaagctggaactgaagcgtgctgatgttgcccctactgtgtccatcttccccccctcctccgagcagctgacatccggcggcgcctccgtggtgtgcttcctgaacaacttctaccccaaagacatcaacgtgaaatggaaaatcgacggctccgagaggcagaacggcgtgctgaactcctggacagaccaggactccaaagactccacatactccatgtcctccacactgacactgacaaaagacgagtacgagaggcacaactcctacacatgcgaggccacacacaaaacatccacatcccccatcgtgaaatccttcaacaggaacgagtgctaataa

SEQ ID NO: 559 - DNA, 660 bp, synthetic construct
  misc_feature 1..660: note = 4A11.2B5 LC nucleic acid
  source 1..660: mol_type = other DNA
gacattgtgatgactcaggcagccttttccaaccctgtaacacttggaactagtgcctccatctcatgccgttccagcaagagtcttttgcacagcgatgggataacctatctctattggtatctgcaacgacccggtcagtcaccccaacttcttatctatcgaatgtctaacttggcctccggcgtgcccgaccgcttttccgggagtgggagcggtacagatttcaccttgcgaatttcccgggttgaggcagaggacgtaggtgtctactattgtgctcagatgcttgagtttcctctcactttcggagccggcaccaaactggaactgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 560 - DNA, 660 bp, synthetic construct; identical to SEQ ID NO: 348
  misc_feature 1..660: note = 4A11.H1L1 LC nucleic acid
  source 1..660: mol_type = other DNA

SEQ ID NO: 561 - DNA, 660 bp, synthetic construct
  misc_feature 1..660: note = 4A11.H4L2 LC nucleic acid
  source 1..660: mol_type = other DNA
gatattgtaatgacccaaagccccctgtccttgcctgttaccccaggtgagcctgccagcatatcttgccgatccagtaaatctctgcttcatagcgatggcatcacctatttgtattggtatttgcagaagcccggccagagcccccagttgctgatctacagaatgtccaaccttgctagtggagtgcccgaccggtttagcggttcaggctcagggactgatttcactcttaaaatcagtagggtagaggccgaagacgttggtgtatattattgtgctcagatgttggagtttccactcacatttggacaaggtacaaagctggaaattaagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 562 - DNA, 660 bp, synthetic construct
  misc_feature 1..660: note = 4G2.2G6 LC nucleic acid
  source 1..660: mol_type = other DNA
gatgtcgtaatgactcaaacccccctgacccttagtgttactataggccagcccgcaagcatctcctgcaaatcctcccagtctcttctttatactgatggcaaaacctacttgtcctggtttttgcagcgcccaggtcaaagtcctaaacgtcttatttacttggtctctaaactcgattccggcgtgcctgaccggttctccggtagcggtagcggtaccgatttcactttgaagattagccgagttgaggcagaagacctcggggtctactactgccttcagagtacacacttcccacttacatttggcgcaggaaccaagttggaggttaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 563 - DNA, 645 bp, synthetic construct
  misc_feature 1..645: note = 6B3.2D3 LC nucleic acid
  source 1..645: mol_type = other DNA
agtattgtgatgacccagactcccaaattcctgcttgtatcagcaggagacagggttaccataacctgcaaggccagtcagagtgtgagtaatgatgtagcttggtaccaacagaagccagggcagtctcctaaattgttgatatattatgcatccaatcgctacactggagtccctgatcgcttcactggcagtggatatgggacggatttcactttcaccatcagcactgtgcaggctgaagacctggcagtttatttctgtcagcaggattatacctctccgtacacgttcggaggggggaccaagctggaaataaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 564 - DNA, 645 bp, synthetic construct
  misc_feature 1..645: note = 6H6.2D6 LC nucleic acid
  source 1..645: mol_type = other DNA
gacatccagatgacccagaccacctcctccctgtccgcctccctgggcgacagggtgaccatctcctgctccgcctcccagggcatctccaactacctgaactggtaccagcagaagcccgacggcaccgtgaagctgctgatctactacacctcctccctgcactccggcgtgccctccaggttctccggctccggctccggcaccgactactccctgaccatctccaacctggagcccgaggacatcgccacctactactgccagcagtactccgagctgccctacaccttcggctccggcaccaagctggagatcaagaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 565 - DNA, 645 bp, synthetic construct
  misc_feature 1..645: note = 9H2.2H10 LC nucleic acid
  source 1..645: mol_type = other DNA
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattataccactccgctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 566 - DNA, 645 bp, synthetic construct
  misc_feature 1..645: note = 13G4.2F8 LC nucleic acid
  source 1..645: mol_type = other DNA
gacattgtgatgacccagtctcaaaaattcatgtccacatcagtaggagagagggtcagcatcacctgcaaggccagtcagaatgtaggtactaatgtagcctggtatcagcagaaagcagggcagtctcttgaactgctgatctatggggcatccaaccggcacactggagtccctgatcgcttcacaggcagtggatctgggacagatttcactctcaccatcaccaatgtgcagtctgaagacatgacaaattatttctgtgagcaatatagcaactttcctctcacgttcggtgctgggaccaagctggagctgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 567 - DNA, 660 bp, synthetic construct
  misc_feature 1..660: note = 13H12.2F8 LC nucleic acid
  source 1..660: mol_type = other DNA
gacgttgttatgacacaaacaccacttacactttctgtaactatcggtcagcctgcctcaatttcatgtaagtcttctcagtccctgtttcatagcgacgggaagacttacctgaattggcttctccaacgaccaggccaatcacccaagcgccttatttatttggtcagtaaattggacagtggagttcccgatagatttaccgggagcggttctgggaccgatttcaccccaaagatctccagggtagaagacgaagatctcggtgtgtactattgctggcaggggacacatttccctctgacattcggtgctggaaccaaacttgagatgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 568 - DNA, 645 bp, synthetic construct
  misc_feature 1..645: note = 15G7.2A7 LC nucleic acid
  source 1..645: mol_type = other DNA
gacatccagatgaaccagtctccatccagtctgtctgcatccctcggagacacaatttccatcacttgccgtgccagtcagaacattaatatttggttaagctggtaccagcagaaaccaggaaatattcctcaactattgatctataaggcttccaacttgcacacaggcgtcccctcaaggtttagtggcagtggatctggaacagatttcacattaaccatcagcagtctgcagcctgaagacattgccacttactactgtctacagggtcaaagttatcctctcacgttcggtgctgggaccaagctggtgatgaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 569 - DNA, 654 bp, synthetic construct
  misc_feature 1..654: note = 19D9.2E5 LC nucleic acid
  source 1..654: mol_type = other DNA
gatgttgtgatgacccagactccactcactttgtcggttaccattggacaaccagcctccatctcttgtaagtcaagtcagagcctcttacatagtgatggaaagacatatttgaattggttgttacagaggccaggccagcctccaaagcgcctaatgtatctggtgtctacactggactctggagtccctgacaggttcactggcagtggatcagggacagatttcacactgaaaatcagcagagtggaggctgaggatttgggagtttattattgctggcaaggtacacattttcctctcacgttcggtgctgggaccaagccggagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 570 - DNA, 654 bp, synthetic construct
  misc_feature 1..654: note = 23B10.2B12 LC nucleic acid
  source 1..654: mol_type = other DNA
gatattgtgatgacgcaggctgcattctccaatccagtcactcttggaacgtcagcttccatctcctgcaggtctagtaagagtctcctacatagtgatggcatcacttatttctattggtatctgcagaggccaggccagtctcctcagctcctgatatatcggatgtccaaccttgcctcaggagtcccagacaggttcagtggcagtgggtcaggaactgatttcacactgagaatcagcagagtggaggctgaggatgtgggtgtttattactgtgctcaaatgctagaattcccgctcacgttcggtgctgggaccaagctggagcggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 571 - DNA, 645 bp, synthetic construct
  misc_feature 1..645: note = 24D12.2H9 LC nucleic acid
  source 1..645: mol_type = other DNA
gacattgtgatgacccagtctcacaaattcatgtccacatcagtaggagacagggtcagcatcacctgcaaggccagtcaggatgtgcgtactgctgtagcctggtatcaacagaaaccagggcaatctcctaaactactgatttactgggcatccacccggcacactggagtccctgatcgcttcacaggcagtggatctgggacagatttcactctcacccttagcaatgtgcagtctgaagacttggcagattatttctgtcagcaatatagtagttatccgtggacgttcggtggaggcaccaagctggaaatcaaacggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaa

SEQ ID NO: 572 - DNA, 21 bp, synthetic construct
  misc_feature 1..21: note = aSMA forward primer
  source 1..21: mol_type = other DNA
agccaagcactgtcaggaatc

SEQ ID NO: 573 - DNA, 20 bp, synthetic construct
  misc_feature 1..20: note = aSMA reverse primer
  source 1..20: mol_type = other DNA
gggcaacacgaagctcattg

SEQ ID NO: 574 - DNA, 20 bp, synthetic construct
  misc_feature 1..20: note = ColA1 forward primer
  source 1..20: mol_type = other DNA
caaagaaggcggcaaaggtc

SEQ ID NO: 575 - DNA, 20 bp, synthetic construct
  misc_feature 1..20: note = ColA1 reverse primer
  source 1..20: mol_type = other DNA
ccctcacgtccagattcacc

SEQ ID NO: 576 - DNA, 19 bp, synthetic construct
  misc_feature 1..19: note = CTGF forward primer
  source 1..19: mol_type = other DNA
tccacccgggttaccaatg

SEQ ID NO: 577 - DNA, 20 bp, synthetic construct
  misc_feature 1..20: note = CTGF reverse primer
  source 1..20: mol_type = other DNA
ccgggacagttgtaatggca

SEQ ID NO: 578 - DNA, 20 bp, synthetic construct
  misc_feature 1..20: note = FN forward primer
  source 1..20: mol_type = other DNA
cacctctgtgcagaccacat

SEQ ID NO: 579 - DNA, 20 bp, synthetic construct
  misc_feature 1..20: note = FN reverse primer
  source 1..20: mol_type = other DNA
acgtcctgccattgtaggtg

SEQ ID NO: 580 - DNA, 20 bp, synthetic construct
  misc_feature 1..20: note = Gal3 forward primer
  source 1..20: mol_type = other DNA
acaattctgggcacggtgaa

SEQ ID NO: 581 - DNA, 20 bp, synthetic construct
  misc_feature 1..20: note = Gal3 reverse primer
  source 1..20: mol_type = other DNA
cgtgggttaaagtggaaggc

SEQ ID NO: 582 - protein, 20 aa, synthetic construct
  REGION 1..20: note = Gal3 Peptide A
PGAYPGQAPPGAYPGAPGAY

SEQ ID NO: 583 - protein, 20 aa, synthetic construct
  REGION 1..20: note = Gal3 Peptide B
GAYPGAPGAYPGAPAPGVYP

SEQ ID NO: 584 - protein, 20 aa, synthetic construct
  REGION 1..20: note = Gal3 Peptide C
PGAPAPGVYPGPPSGPGAYP

SEQ ID NO: 585 - protein, 20 aa, synthetic construct
  REGION 1..20: note = Gal3 Peptide D
GPPSGPGAYPSSGQPSATGA

SEQ ID NO: 586 - protein, 20 aa, synthetic construct
  REGION 1..20: note = Gal3 Peptide E
SSGQPSATGAYPATGPYGAP

SEQ ID NO: 587 - protein, 20 aa, synthetic construct
  REGION 1..20: note = Gal3 Peptide F
YPATGPYGAPAGPLIVPYNL

SEQ ID NO: 588 - protein, 8 aa, synthetic construct
  REGION 1..8: note = VH_CDR1_24
GFTFSNYW

SEQ ID NO: 589 - protein, 8 aa, synthetic construct
  REGION 1..8: note = VH_CDR1_25
GYTFTSYV

SEQ ID NO: 590 - protein, 8 aa, synthetic construct
  REGION 1..8: note = VH_CDR1_26
GYTFTSYD

SEQ ID NO: 591 - protein, 8 aa, synthetic construct
  REGION 1..8: note = VH_CDR1_27
GYTFTSYD

SEQ ID NO: 592 - protein, 8 aa, synthetic construct
  REGION 1..8: note = VH_CDR1_28
GYSFTNYI

SEQ ID NO: 593 - protein, 8 aa, synthetic construct
  REGION 1..8: note = VH_CDR1_29
GYTFRSYD

SEQ ID NO: 594 - protein, 8 aa, synthetic construct
  REGION 1..8: note = VH_CDR1_30
GFTFSNFW

SEQ ID NO: 595 - protein, 8 aa, synthetic construct
  REGION 1..8: note = VH_CDR1_31
GYTFTSYD

SEQ ID NO: 596 - protein, 8 aa, synthetic construct
  REGION 1..8: note = VH_CDR1_32
EYAFNNYM

SEQ ID NO: 597 - protein, 8 aa, synthetic construct
  REGION 1..8: note = VH_CDR1_33
GYSFTGYT

SEQ ID NO: 598 - protein, 8 aa, synthetic construct
  REGION 1..8: note = VH_CDR1_34
GYTFTNYW

SEQ ID NO: 599 - protein, 5 aa, synthetic construct
  REGION 1..5: note = VH_CDR1_35
SSVTY

SEQ ID NO: 600 - protein, 8 aa, synthetic construct
  REGION 1..8: note = VH_CDR1_36
GYTFNNYW

SEQ ID NO: 601 - protein, 8 aa, synthetic construct
  REGION 1..8: note = VH_CDR1_37
GYTFTNYW

SEQ ID NO: 602 - protein, 8 aa, synthetic construct
  REGION 1..8: note = VH_CDR1_38
GFSLTSYG

SEQ ID NO: 603 - protein, 8 aa, synthetic construct
  REGION 1..8: note = VH_CDR1_39
GYTFTNYG

SEQ ID NO: 604 - protein, 8 aa, synthetic construct
  REGION 1..8: note = VH_CDR1_40
GFSLTSYG

SEQ ID NO: 605 - protein, 8 aa, synthetic construct
  REGION 1..8: note = VH_CDR1_41
GFSLINYG

SEQ ID NO: 606 - protein, 8 aa, synthetic construct
  REGION 1..8: note = VH_CDR1_42
GFSLTSYG

SEQ ID NO: 607 - protein, 8 aa, synthetic construct
  REGION 1..8: note = VH_CDR1_43
GFSLSSYG

SEQ ID NO: 608 - protein, 8 aa, synthetic construct
  REGION 1..8: note = VH_CDR1_44
GYSFTNYW

SEQ ID NO: 609 - protein, 8 aa, synthetic construct
  REGION 1..8: note = VH_CDR1_45
GYSFTSYW

SEQ ID NO: 610 - protein, 9 aa, synthetic construct
  REGION 1..9: note = VH_CDR1_46
GYSITSGYY

SEQ ID NO: 611 - protein, 10 aa, synthetic construct
  REGION 1..10: note = VH_CDR1_47
GFSLSTFGMG

SEQ ID NO: 612 - protein, 9 aa, synthetic construct
  REGION 1..9: note = VH_CDR1_48
GYSITSGYY

SEQ ID NO: 613 - protein, 8 aa, synthetic construct
  REGION 1..8: note = VH_CDR1_49
GFNIKDYY

SEQ ID NO: 614 - protein, 8 aa, synthetic construct
  REGION 1..8: note = VH_CDR1_50
RYTFTDYN

SEQ ID NO: 615 - protein, 8 aa, synthetic construct
  REGION 1..8: note = VH_CDR1_51
GYTFTIFG

SEQ ID NO: 616 - protein, 10 aa, synthetic construct
  REGION 1..10: note = VH_CDR2_26
LRLKSNNYAT

SEQ ID NO: 617 - protein, 8 aa, synthetic construct
  REGION 1..8: note = VH_CDR2_27
INPYNDGT

SEQ ID NO: 618 - protein, 8 aa, synthetic construct
  REGION 1..8: note = VH_CDR2_28
IYPGDGST

SEQ ID NO: 619 - protein, 8 aa, synthetic construct
  REGION 1..8: note = VH_CDR2_29
IYPGDGST

SEQ ID NO: 620 - protein, 8 aa, synthetic construct
  REGION 1..8: note = VH_CDR2_30
INPYNDGT

SEQ ID NO: 621 - protein, 8 aa, synthetic construct
  REGION 1..8: note = VH_CDR2_31
IFPGEGIS

SEQ ID NO: 622 - protein, 10 aa, synthetic construct
  REGION 1..10: note = VH_CDR2_32
IRLKSNNYAT

SEQ ID NO: 623 - protein, 8 aa, synthetic construct
  REGION 1..8: note = VH_CDR2_33
IFPGDGTT

SEQ ID NO: 624 - protein, 8 aa, synthetic construct
  REGION 1..8: note = VH_CDR2_34
INPGSGAT

SEQ ID NO: 625 - protein, 8 aa, synthetic construct
  REGION 1..8: note = VH_CDR2_35
INPYNGGT

SEQ ID NO: 626 - protein, 8 aa, synthetic construct
  REGION 1..8: note = VH_CDR2_36
FLPGDGSS

SEQ ID NO: 627 - skipped

SEQ ID NO: 628 - protein, 8 aa, synthetic construct
  REGION 1..8: note = VH_CDR2_38
IDPSDSET

SEQ ID NO: 629 - protein, 8 aa, synthetic construct
  REGION 1..8: note = VH_CDR2_39
IDPSDSET

SEQ ID NO: 630 - protein, 7 aa, synthetic construct
  REGION 1..7: note = VH_CDR2_40
IWAGGST

SEQ ID NO: 631 - protein, 8 aa, synthetic construct
  REGION 1..8: note = VH_CDR2_41
INTNTGEP

SEQ ID NO: 632 - protein, 7 aa, synthetic construct
  REGION 1..7: note = VH_CDR2_42
IWAGGST

SEQ ID NO: 633 - protein, 7 aa, synthetic construct
  REGION 1..7: note = VH_CDR2_43
IWSGGST

SEQ ID NO: 634 - protein, 7 aa, synthetic construct
  REGION 1..7: note = VH_CDR2_44
IWAGGST

SEQ ID NO: 635 - protein, 7 aa, synthetic construct
  REGION 1..7: note = VH_CDR2_45
IWAGGST

SEQ ID NO: 636 - protein, 8 aa, synthetic construct
  REGION 1..8: note = VH_CDR2_46
IDPSDSET

SEQ ID NO: 637 - protein, 8 aa, synthetic construct
  REGION 1..8: note = VH_CDR2_47
IDPSDSET

SEQ ID NO: 638 - protein, 7 aa, synthetic construct
  REGION 1..7: note = VH_CDR2_48
ISYDGRN

SEQ ID NO: 639 - protein, 7 aa, synthetic construct
  REGION 1..7: note = VH_CDR2_49
IWWDDDK

SEQ ID NO: 640 - protein, 7 aa, synthetic construct
  REGION 1..7: note = VH_CDR2_50
ISYDGRN

SEQ ID NO: 641 - protein, 8 aa, synthetic construct
  REGION 1..8: note = VH_CDR2_51
IYPEDDET

SEQ ID NO: 642 - protein, 8 aa, synthetic construct
  REGION 1..8: note = VH_CDR2_52
IYPNNGGT

SEQ ID NO: 643 - protein, 8 aa, synthetic construct
  REGION 1..8: note = VH_CDR2_53
VNTYTGEP

SEQ ID NO: 644 - protein, 13 aa, synthetic construct
  REGION 1..13: note = VH_CDR3_34
TRHYGFPYWYFDV

SEQ ID NO: 645 - protein, 11 aa, synthetic construct
  REGION 1..11: note = VH_CDR3_35
ARLLFYYAMDY

SEQ ID NO: 646 - protein, 11 aa, synthetic construct
  REGION 1..11: note = VH_CDR3_36
AREAASNAMDY

SEQ ID NO: 647 - protein, 11 aa, synthetic construct
  REGION 1..11: note = VH_CDR3_37
AREAASNAMDY

SEQ ID NO: 648 - protein, 11 aa, synthetic construct
  REGION 1..11: note = VH_CDR3_38
ARLLFYYAMDY

SEQ ID NO: 649 - protein, 11 aa, synthetic construct
  REGION 1..11: note = VH_CDR3_39
AREATAGAMDY

SEQ ID NO: 650 - protein, 13 aa, synthetic construct
  REGION 1..13: note = VH_CDR3_40
TRHYGFPYWYFDV

SEQ ID NO: 651 - protein, 11 aa, synthetic construct
  REGION 1..11: note = VH_CDR3_41
AREGAGSWFAY

SEQ ID NO: 652 - protein, 7 aa, synthetic construct
  REGION 1..7: note = VH_CDR3_42
TYNDLGY

SEQ ID NO: 653 - protein, 16 aa, synthetic construct
  REGION 1..16: note = VH_CDR3_43
ARKDDYYGSSPYALDY

SEQ ID NO: 654 - protein, 9 aa, synthetic construct
  REGION 1..9: note = VH_CDR3_44
VRDDYDSDY

SEQ ID NO: 655 - protein, 9 aa, synthetic construct
  REGION 1..9: note = VH_CDR3_45
QQWSSNPLT

SEQ ID NO: 656 - protein, 15 aa, synthetic construct
  REGION 1..15: note = VH_CDR3_46
ARGDYYYGTRGPMDY

SEQ ID NO: 657 - protein, 8 aa, synthetic construct
  REGION 1..8: note = VH_CDR3_47
ARWGAMDY

SEQ ID NO: 658 - protein, 15 aa, synthetic construct
  REGION 1..15: note = VH_CDR3_48
ARDRASLLRPGAMDY

SEQ ID NO: 659 - protein, 11 aa, synthetic construct
  REGION 1..11: note = VH_CDR3_49
ARLDYDYDEDY

SEQ ID NO: 660 - protein, 14 aa, synthetic construct
  REGION 1..14: note = VH_CDR3_50
ASPPSGYDYDVFAS

SEQ ID NO: 661 - protein, 14 aa, synthetic construct
  REGION 1..14: note = VH_CDR3_51
ARIYYYGRGYAMDY

SEQ ID NO: 662 - protein, 14 aa, synthetic construct
  REGION 1..14: note = VH_CDR3_52
ASPPSGYDYDVFAY

SEQ ID NO: 663 - protein, 14 aa, synthetic construct
  REGION 1..14: note = VH_CDR3_53
AKPPYDYDGAWFAY

SEQ ID NO: 664 - protein, 7 aa, synthetic construct
  REGION 1..7: note = VH_CDR3_54
ATGRPDY

SEQ ID NO: 665 - protein, 7 aa, synthetic construct
  REGION 1..7: note = VH_CDR3_55
AKGRPDY

SEQ ID NO: 666 - protein, 11 aa, synthetic construct
  REGION 1..11: note = VH_CDR3_56
AGYDFLWYFDV

SEQ ID NO: 667 - protein, 13 aa, synthetic construct
  REGION 1..13: note = VH_CDR3_57
ARIDYGDYVGFAY

SEQ ID NO: 668 - protein, 11 aa, synthetic construct
  REGION 1..11: note = VH_CDR3_58
AGYDFLWYFDV

SEQ ID NO: 669 - protein, 12 aa, synthetic construct
  REGION 1..12: note = VH_CDR3_59
ARSGGYYVWFAY

SEQ ID NO: 670 - protein, 14 aa, synthetic construct
  REGION 1..14: note = VH_CDR3_60
ARSYYDGSYDAMDY

SEQ ID NO: 671 - protein, 7 aa, synthetic construct
  REGION 1..7: note = VH_CDR3_61
ARGWFAY

SEQ ID NO: 672 - protein, 10 aa, synthetic construct
  REGION 1..10: note = VL_CDR1_33
ESVDSYGNSF

SEQ ID NO: 673 - protein, 6 aa, synthetic construct
  REGION 1..6: note = VL_CDR1_34
QGINSN

SEQ ID NO: 674 - protein, 10 aa, synthetic construct
  REGION 1..10: note = VL_CDR1_35
KSVSTSGYSY

SEQ ID NO: 675 - protein, 10 aa, synthetic construct
  REGION 1..10: note = VL_CDR1_36
KSVSTSGYSY

SEQ ID NO: 676 - protein, 6 aa, synthetic construct
  REGION 1..6: note = VL_CDR1_37
QGISSN

SEQ ID NO: 677 - protein, 10 aa, synthetic construct
  REGION 1..10: note = VL_CDR1_38
KSVSTSGYSC

SEQ ID NO: 678 - protein, 10 aa, synthetic construct
  REGION 1..10: note = VL_CDR1_39
ESVDSYGNSF

SEQ ID NO: 679 - protein, 10 aa, synthetic construct
  REGION 1..10: note = VL_CDR1_40
KSVSTSGYSY

SEQ ID NO: 680 - protein, 11 aa, synthetic construct
  REGION 1..11: note = VL_CDR1_41
QSLLYSDGKTY

SEQ ID NO: 681 - protein, 10 aa, synthetic construct
  REGION 1..10: note = VL_CDR1_42
ESVENHVNSF

SEQ ID NO: 682 - protein, 9 aa, synthetic construct
  REGION 1..9: note = VL_CDR1_43
TGAVTTSNY

SEQ ID NO: 683 - protein, 8 aa, synthetic construct
  REGION 1..8: note = VL_CDR1_44
TYMYWYQQ

SEQ ID NO: 684 - protein, 10 aa, synthetic construct
  REGION 1..10: note = VL_CDR1_45
ESLDNSGISF

SEQ ID NO: 685 - protein, 6 aa, synthetic construct
  REGION 1..6: note = VL_CDR1_46
QDVSTT

SEQ ID NO: 686 - protein, 6 aa, synthetic construct
  REGION 1..6: note = VL_CDR1_47
QNVGIN

SEQ ID NO: 687 - protein, 11 aa, synthetic construct
  REGION 1..11: note = VL_CDR1_48
QSLLDSDGKTY

SEQ ID NO: 688 - protein, 6 aa, synthetic construct
  REGION 1..6: note = VL_CDR1_49
GNIHNY

SEQ ID NO: 689 - protein, 5 aa, synthetic construct
  REGION 1..5: note = VL_CDR1_50
SSVTY

SEQ ID NO: 690 - protein, 6 aa, synthetic construct
  REGION 1..6: note = VL_CDR1_51
GNIHNY

SEQ ID NO: 691 - protein, 6 aa, synthetic construct
  REGION 1..6: note = VL_CDR1_52
QSVSSD

SEQ ID NO: 692 - protein, 6 aa, synthetic construct
  REGION 1..6: note = VL_CDR1_53
QDVSTA

SEQ ID NO: 693 - protein, 6 aa, synthetic construct
  REGION 1..6: note = VL_CDR1_54
QDVSTA

SEQ ID NO: 694 - protein, 11 aa, synthetic construct
  REGION 1..11: note = VL_CDR1_55
QSLLYINGKTY

SEQ ID NO: 695 - protein, 6 aa, synthetic construct
  REGION 1..6: note = VL_CDR1_56
QGINSN

SEQ ID NO: 696 - protein, 11 aa, synthetic construct
  REGION 1..11: note = VL_CDR1_57
QSLLYINGKTY

SEQ ID NO: 697 - protein, 11 aa, synthetic construct
  REGION 1..11: note = VL_CDR1_58
RSLLHSNGYTY

SEQ ID NO: 698 - protein, 6 aa, synthetic construct
  REGION 1..6: note = VL_CDR1_59
QGISNY

SEQ ID NO: 699 - protein, 11 aa, synthetic construct
  REGION 1..11: note = VL_CDR1_60
RSLLDSDGKTY

SEQ ID NO: 700-710 - skipped (11 entries)

SEQ ID NO: 711 - protein, 7 aa, synthetic construct
  REGION 1..7: note = VL_CDR2_30
SGVPARF

SEQ ID NO: 712-727 - skipped (16 entries)

SEQ ID NO: 728 - protein, 9 aa, synthetic construct
  REGION 1..9: note = VL_CDR3_28
QQNNEDPWT

SEQ ID NO: 729 - protein, 9 aa, synthetic construct
  REGION 1..9: note = VL_CDR3_29
VQYDQFPWT

SEQ ID NO: 730 - protein, 9 aa, synthetic construct
  REGION 1..9: note = VL_CDR3_30
QHSRELPLT

SEQ ID NO: 731 - protein, 9 aa, synthetic construct
  REGION 1..9: note = VL_CDR3_31
QHSRELPLT

SEQ ID NO: 732 - protein, 9 aa, synthetic construct
  REGION 1..9: note = VL_CDR3_32
VQYDQFPWT

SEQ ID NO: 733 - protein, 9 aa, synthetic construct
  REGION 1..9: note = VL_CDR3_33
HHSTELPYT

SEQ ID NO: 734 - protein, 9 aa, synthetic construct
  REGION 1..9: note = VL_CDR3_34
QQNNEDPWT

SEQ ID NO: 735 - protein, 9 aa, synthetic construct
  REGION 1..9: note = VL_CDR3_35
QHSRELPLT

SEQ ID NO: 736 - protein, 8 aa, synthetic construct
  REGION 1..8: note = VL_CDR3_36
VQGTHLPT

SEQ ID NO: 737 - protein, 9 aa, synthetic construct
  REGION 1..9: note = VL_CDR3_37
QQNNEVPYT

SEQ ID NO: 738 - protein, 9 aa, synthetic construct
  REGION 1..9: note = VL_CDR3_38
ALWYSNHLV

SEQ ID NO: 739 - protein, 14 aa, synthetic construct
  REGION 1..14: note = VL_CDR3_39
ARIYYYGRGYAMDY

SEQ ID NO: 740 - protein, 9 aa, synthetic construct
  REGION 1..9: note = VL_CDR3_40
QHSKEVPWT

SEQ ID NO: 741 - protein, 9 aa, synthetic construct
  REGION 1..9: note = VL_CDR3_41
QQHYSPPFT

SEQ ID NO: 742 - protein, 9 aa, synthetic construct
  REGION 1..9: note = VL_CDR3_42
QQYNSYPYT

SEQ ID NO: 743 - protein, 9 aa, synthetic construct
  REGION 1..9: note = VL_CDR3_43
WQGTHFPFT

SEQ ID NO: 744 - protein, 9 aa, synthetic construct
  REGION 1..9: note = VL_CDR3_44
QHFWSIFPT

SEQ ID NO: 745 - protein, 9 aa, synthetic construct
  REGION 1..9: note = VL_CDR3_45
QQWSSNPLT

SEQ ID NO: 746 - protein, 9 aa, synthetic construct
  REGION 1..9: note = VL_CDR3_46
QHFWSLFPT

SEQ ID NO: 747 - protein, 9 aa, synthetic construct
  REGION 1..9: note = VL_CDR3_47
QHNYISPFT

SEQ ID NO: 748 - protein, 9 aa, synthetic construct
  REGION 1..9: note = VL_CDR3_48
QQHYSTPYT

SEQ ID NO: 749 - protein, 9 aa, synthetic construct
  REGION 1..9: note = VL_CDR3_49
QQHYNTPYT

SEQ ID NO: 750 - protein, 9 aa, synthetic construct
  REGION 1..9: note = VL_CDR3_50
LQSTHFPQT

SEQ ID NO: 751 - protein, 9 aa, synthetic construct
  REGION 1..9: note = VL_CDR3_51
VQYDQFPWT

SEQ ID NO: 752 - protein, 9 aa, synthetic construct
  REGION 1..9: note = VL_CDR3_52
LQSTHFPQT

SEQ ID NO: 753 - protein, 9 aa, synthetic construct
  REGION 1..9: note = VL_CDR3_53
MQHLEYPYT

SEQ ID NO: 754 - protein, 9 aa, synthetic construct
  REGION 1..9: note = VL_CDR3_54
QQYSKLPRT

SEQ ID NO: 755 - protein, 9 aa, synthetic construct
  REGION 1..9: note = VL_CDR3_55
WQGTHFPQT

SEQ ID NO: 756 - protein, 122 aa, synthetic construct
  REGION 1..122: note = VH-44
EVKLEESGGGLVQPGGSMKLSCVASGFTFSNYWMNWVRQSPEKGLEWVAELRLKSNNYATHYAESVKGRFTISRDDSKSSVYLQMNNLRAEDTGIYYCTRHYGFPYWYFDVWGAGTTVTVSS

SEQ ID NO: 757 - protein, 118 aa, synthetic construct
  REGION 1..118: note = VH-45
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARLLFYYAMDYWGQGTSVTVSS

SEQ ID NO: 758 - protein, 118 aa, synthetic construct
  REGION 1..118: note = VH-46
QVQLQQSGPELVRPGALVKISCKASGYTFTSYDINWVKQRPGQGLEWIGWIYPGDGSTKYNEKFKGKATLTADKSSSTVYMQLSSLTSENSAVYFCAREAASNAMDYWGQGTSVTVSS

SEQ ID NO: 759 - protein, 118 aa, synthetic construct
  REGION 1..118: note = VH-47
QVQLQQSGPELVKPGALVKISCKASGYTFTSYDINWVKQRPGQGLEWIGWIYPGDGSTKYNEKFKGKAALTADKSSSTAYMQLSSLTSENSAVYFCAREAASNAMDYWGQGTSVTVSS

SEQ ID NO: 760 - protein, 118 aa, synthetic construct
  REGION 1..118: note = VH-48
EVQLQQSGPELVKPGASVKMSCKASGYSFTNYIMHWVKQKPGQGLEWIGYINPYNDGTKYNEKFKGKATLTSDKSSSTAYMELSSLTSEDSAVYYCARLLFYYAMDYWGQGTSVTVSS

SEQ ID NO: 761 - protein, 118 aa, synthetic construct
  REGION 1..118: note = VH-49
QVQLQQSGAELVKPGASVKLSCKASGYTFRSYDINWVRQRPEQGLEWIGWIFPGEGISKYNEKFKGKATLTTDKLSSTAYMQLSSLTSEDSAVYFCAREATAGAMDYWGQGTSVTVSS

SEQ ID NO: 762 - protein, 122 aa, synthetic construct
  REGION 1..122: note = VH-50
EVKLLESGGGLVQPGGSMKLSCVASGFTFSNFWMNWVRQSPEKGLEWVAEIRLKSNNYATHYAESVKGRFTISRDDSKSSVYLQMNNLRAEDTGIYYCTRHYGFPYWYFDVWGAGTTVTVSS

SEQ ID NO: 763 - protein, 118 aa, synthetic construct
  REGION 1..118: note = VH-51
QIQLQQSGAELVKPGASVKLSCKASGYTFTSYDICWVRQRPEQGLEWIGWIFPGDGTTKYTENFKGKATLTTDKSSSTAYMHLSRLTSEDSAVYFCAREGAGSWFAYWGQGTLVTVSA

SEQ ID NO: 764 - protein, 114 aa, synthetic construct
  REGION 1..114: note = VH-52
EVQLQQSGAELVRPGTSVKVSCKASEYAFNNYMMEWVKQRPGQGLEWIGVINPGSGATNYNENFKDKATLTADKSSTTAYMQLSSLTSDDSAVYFCTYNDLGYWGQGTLVTVSA

SEQ ID NO: 765 - protein, 123 aa, synthetic construct
  REGION 1..123: note = VH-53
EVKLQQSGPELMKPGTSMKISCKASGYSFTGYTMNWVKQSHGKNLEWIGLINPYNGGTNYNQKFRGKATLSVDKSSNTAYMELLSLTSEDSAVYYCARKDDYYGSSPYALDYWGQGTSVTVSS

SEQ ID NO: 766 - protein, 116 aa, synthetic construct
  REGION 1..116: note = VH-54
QVQLQQSGAELMKPGASVKISCKATGYTFTNYWIEWVKQRPGHGLEWIGEFLPGDGSSHYNEKFKGKATFTSDTSSNTAYIHLTSLTSEDSAVYYCVRDDYDSDYWAQGTTLTVSS

SEQ ID NO: 767 - protein, 106 aa, synthetic construct
  REGION 1..106: note = VH-55
QIVLSQSPAILSASPGEKVTMTCRASSSVTYMYWYQQKPGSSPQPWIYATSNLASGVPARFSGSGSGTSYSLTISRVEAEDAATYYCQQWSSNPLTFGAGTKLELK

SEQ ID NO: 768 - protein, 122 aa, synthetic construct
  REGION 1..122: note = VH-56
EVQLQQSGAEVVKPGAPVKLSCKASGYTFNNYWLNWVKQRPGRGLEWIGRIDPSDSETHYNQKFKDKATLTVDMSSSTAYIQLSSLTSEDSAVYYCARGDYYYGTRGPMDYWGQGTSVTVSS

SEQ ID NO: 769 - protein, 115 aa, synthetic construct
  REGION 1..115: note = VH-57
QVQLQQSGAELVRPGASVNLSCKASGYTFTNYWMNWVKQRPGQGLEWVGMIDPSDSETHYNQMFKDKATLTIDKSSSIAYMQLSSLTSEDSAVYYCARWGAMDYWGQGTSVTVSS

SEQ ID NO: 770 - protein, 121 aa, synthetic construct
  REGION 1..121: note = VH-58
QVQLKQSGPGLVAPSQSLSITCTVSGFSLTSYGVHWVRQPPGKGLEWLGIIWAGGSTNYNPALMSRLSISKDKSKSQVFLKMNSLKTDDTAMYYCARDRASLLRPGAMDYWGQGTSVTVSS

SEQ ID NO: 771 - protein, 118 aa, synthetic construct
  REGION 1..118: note = VH-59
QIQLVQSGPELKKPGETVKISCKASGYTFTNYGMNWVKQAPGKGLKWMGWINTNTGEPTYAEEFKGRFAFSLETSASTAYLQINYLKNEDTATFFCARLDYDYDEDYWGQGTTLTVSS

SEQ ID NO: 772 - protein, 120 aa, synthetic construct
  REGION 1..120: note = VH-60
QVQLVETGPGLVAPSQSLSITCTVSGFSLTSYGVHWIRQSPGKGLEWLGTIWAGGSTDYNSPLMSRLSISKDNSKNQVFLKMNSLQIDDTAIYYCASPPSGYDYDVFASWGQGTLVTVSA

SEQ ID NO: 773 - protein, 120 aa, synthetic construct
  REGION 1..120: note = VH-61
QVQLKQSGPGLVQPSQSLSITCTVSGFSLINYGVHWVRQSPGKGLEWLGVIWSGGSTDYNAAFISRLSISKDNSKSQVFFKMNSLQADDTAIYYCARIYYYGRGYAMDYWGQGTSVTVSS

SEQ ID NO: 774 - protein, 120 aa, synthetic construct
  REGION 1..120: note = VH-62
QVQLKESGPGLVAPSQSLSITCTVSGFSLTSYGVHWIRQPPGKGLEWLGGIWAGGSTDYNSPLMSRLSITKDNSKSQVFLKMNSLQSDDTAMYYCASPPSGYDYDVFAYWGQGTLVTVSA

SEQ ID NO: 775 - protein, 120 aa, synthetic construct
  REGION 1..120: note = VH-63
QVQLKESGPVLVAPSQSLSITCTVSGFSLSSYGVHWVRQPPGKGLEWLGVIWAGGSTDYDSALMSRLSISKDNSKSQVFLKMDSLQTDDTAMYYCAKPPYDYDGAWFAYWGQGTLVTVSA

SEQ ID NO: 776 - protein, 114 aa, synthetic construct
  REGION 1..114: note = VH-64
QVQLQQSGPQLVRPGASVKISCKASGYSFTNYWMHWVKQRPGQGLEWIGMIDPSDSETRLNQKFKDKATLTVDKSSSTAYMQLSSPTSEDSAVYYCATGRPDYWGQGTTLTVSS

SEQ ID NO: 777 - protein, 114 aa, synthetic construct
  REGION 1..114: note = VH-65
QVQLQQSGPQLVRPGASVKISCKASGYSFTSYWMHWVKQRPGQGLEWIGMIDPSDSETRLNQKFKDKATLTVDKSSSTAYMQLSSPTSEDSIVYYCAKGRPDYWGQGTTLTVSS

SEQ ID NO: 778 - protein, 118 aa, synthetic construct
  REGION 1..118: note = VH-66
DGKLQESGPGLVKPSQSLSLTCSVTGYSITSGYYWNWIRQFPGNKLEWMGHISYDGRNNYNPSLKNRISITRDTSKNQFFLKLNSVTTEDTATYYCAGYDFLWYFDVWGTGTTVTVSS

SEQ ID NO: 779 - protein, 121 aa, synthetic construct
  REGION 1..121: note = VH-67
QVQLKESGPGILQPSQTLSLTCSFSGFSLSTFGMGVGWIRQPSGKGLEWLAHIWWDDDKHYNPALKSRLTISKDTSKNQVFLRIANVDTADNATYYCARIDYGDYVGFAYWGQGTLVTVSA

SEQ ID NO: 780 - protein, 118 aa, synthetic construct
  REGION 1..118: note = VH-68
DGQLQESGPGLVKPSQSLSLTCSVTGYSITSGYYWNWIRQFPGNKLEWMGHISYDGRNNYNPSLKNRISITRDTSKNQFFLKLNSVTTEDTATYYCAGYDFLWYFDVWGTGTTVTVSS

SEQ ID NO: 781 - protein, 119 aa, synthetic construct
  REGION 1..119: note = VH-69
EVQLQQSGAELVKSGASVKLSCTASGFNIKDYYMHWVKQRTEQGLEWIGRIYPEDDETKYAPKFQGKATITADTSSNTAYLQLSSLTSEDTAVYYCARSGGYYVWFAYWGQGTLVTVSA

SEQ ID NO: 782 - protein, 121 aa, synthetic construct
  REGION 1..121: note = VH-70
EVQLQQSGPELVKPGSSVKISCKASRYTFTDYNMDWVKQSHGKRLEWIGYIYPNNGGTGYNQKFKGKATLTVDKSSSTAYMELRSLTSEDSAVYYCARSYYDGSYDAMDYWGQGTSVTVSS

SEQ ID NO: 783 - protein, 114 aa, synthetic construct
  REGION 1..114: note = VH-71
QIQLVQSGPELKKPGETVKISCKASGYTFTIFGMNWVKQAPGKGLKWMGWVNTYTGEPTYADDFKGRFAFSLETSASTAYLQINNLKNEDTATYFCARGWFAYWGQGTLVTVSA

SEQ ID NO: 784 - protein, 111 aa, synthetic construct; identical to SEQ ID NO: 259
  REGION 1..111: note = VL-44

SEQ ID NO: 785 - protein, 107 aa, synthetic construct
  REGION 1..107: note = VL-45
DILMTQSPSSMSVSLGDTVSITCHASQGINSNIGWLQQKPGKSFKGLIYHGTNLEDGVPSRFSGSGSGADYSLTISSLESEDFADYYCVQYDQFPWTFGGGTKLEIK

SEQ ID NO: 786 - protein, 111 aa, synthetic construct
  REGION 1..111: note = VL-46
DIVMSQSPASLGVSLGQRATISCRASKSVSTSGYSYMHWYQQKPGQPPKLLIYLASILESGVPARFSGSGSGTDFTLNIHPVEEEDAAIYYCQHSRELPLTFGAGTKLELK

SEQ ID NO: 787 - protein, 111 aa, synthetic construct
  REGION 1..111: note = VL-47
DIVLTQSPASLAVSLGQRATISCRASKSVSTSGYSYMHWYQQKPGQPPKLLIYLASILESGVPARFSGSGSGTDFTLNIHPVEEEDAATYYCQHSRELPLTFGAGTKLELK

SEQ ID NO: 788 - protein, 107 aa, synthetic construct
  REGION 1..107: note = VL-48
DILMTQTPSSMSVSLGDTVSITCHASQGISSNIGWLQQKPRKSFKGLIYYGTNLEDGVPSRFSGSGSGADYSLTISSLESEDFADYYCVQYDQFPWTFGGGTKLEIK

SEQ ID NO: 789 - protein, 111 aa, synthetic construct
  REGION 1..111: note = VL-49
DIVLTQSPASLAVSLGQRATISCRASKSVSTSGYSCMHWYQQKPGQPPKLLIYLASNLESGVPARFSGGGSGTDFTLSIHPVEEEDAATYYCHHSTELPYTFGGGTKLEVE

SEQ ID NO: 790 - protein, 111 aa, synthetic construct
  REGION 1..111: note = VL-50
DIVLTQSPASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIK

SEQ ID NO: 791 - protein, 111 aa, synthetic construct; identical to SEQ ID NO: 787
  REGION 1..111: note = VL-51

SEQ ID NO: 792 - protein, 111 aa, synthetic construct
  REGION 1..111: note = VL-52
DVVMTQTPLTLSVTIGQPASISCKSSQSLLYSDGKTYLNWLLQRPGQSPKRLIYLVSQLDSGVPDRFTGSGSGTDFTLKISRVEADDLGVYCCVQGTHLPTFGGGTKLEIK

SEQ ID NO: 793 - protein, 111 aa, synthetic construct
  REGION 1..111: note = VL-53
DIVMTQSPASLAVSLGQRATISCRASESVENHVNSFMHWFQQKPGQPPKLLIYRASNLQSGIPARFSGSGSRTDFTLTINPVEADDVATYYCQQNNEVPYTFGGGTKLEIK

SEQ ID NO: 794 - protein, 109 aa, synthetic construct; identical to SEQ ID NO: 185
  REGION 1..109: note = VL-54

SEQ ID NO: 795 - protein, 120 aa, synthetic construct
  REGION 1..120: note = VL-55
SQSPAILSASPGEKVTMTCRASSSVTYMYWYQQKPGSSPQPWIYATSNLASGVPARFSGSGSGTSYSLTISRVEAEDAATYYCQQWSSNPLTFGAARIYYYGRGYAMDYVFIFPPSDEQL

SEQ ID NO: 796 - protein, 111 aa, synthetic construct
  REGION 1..111: note = VL-56
DIVLTQSPASLAVSLGQRATISCRASESLDNSGISFMNWFQQRPGQPPKLLIYAASNLESGVPARFSGSGSGTDFSLNIHPMEEDDAAMYFCQHSKEVPWTFGGGTKLEIK

SEQ ID NO: 797 - protein, 107 aa, synthetic construct
  REGION 1..107: note = VL-57
DIVMTQSHKIMSTSVGDRVSITCKASQDVSTTVAWFQQKPGQSPKLLLYWASTRHTGVPDRFTGSGSGTDYTLTISSVQAEDLALYYCQQHYSPPFTFGSGTKLEIK

SEQ ID NO: 798 - protein, 107 aa, synthetic construct
  REGION 1..107: note = VL-58
DIVMTQSQKFMSTSVGDRVSVTCKASQNVGINVAWYQQKPGQSPKALIYSASYRYSGVPDRFTGSGSGTDFTLTISNVQSEDLAEYFCQQYNSYPYTFGGGTKLEMK

SEQ ID NO: 799 - protein, 112 aa, synthetic construct
  REGION 1..112: note = VL-59
DVVMTQTPLTLSVTLGQPASISCRSSQSLLDSDGKTYLNWLLQRPGQSPKRLIYLVSRLDSGVPDRFTGSGSGTDFTLKISRVEAEDLGVYYCWQGTHFPFTFGGGTKLEIK

SEQ ID NO: 800 - protein, 107 aa, synthetic construct
  REGION 1..107: note = VL-60
DIRMTQSPASLSASVGETVTITCRASGNIHNYLAWYHQKQGKSPQLLVYNVKTLADGVPSRFSGSGSGTQFSLKINSLQPEDFGTYYCQHFWSIFPTFGGGTKLEIK

SEQ ID NO: 801 - protein, 106 aa, synthetic construct
  REGION 1..106: note = VL-61
QILLSQSPAILSASPGEKVTMTCRASSSVTYMYWYQQKPGSSPQPWIYATSNLASGVPARFSGSGSGTSYSLTISRVEAEDAATYYCQQWSSNPLTFGAGTKLELK

SEQ ID NO: 802 - protein, 107 aa, synthetic construct
  REGION 1..107: note = VL-62
DILMTQSPASLSASVGETVTITCRASGNIHNYLAWYQQKQGKSPQLLVYSAKTLADGVPSRFSGSGSGTQFSLKIDSLQPEDFGTYYCQHFWSLFPTFGGGTKLEIK

SEQ ID NO: 803 - protein, 107 aa, synthetic construct
  REGION 1..107: note = VL-63
DVVMTQTPKFLLVSAGDRVTITCKASQSVSSDVAWYQQKPGQSPKLLIYFASNRYTGVPDRFTGSGYGTDFTFTISTVQAEDLAVYFCQHNYISPFTFGGGTKLEIK

SEQ ID NO: 804 - protein, 107 aa, synthetic construct
  REGION 1..107: note = VL-64
DIVMTQSHKFMSTSVGDRVSITCKASQDVSTAVAWYQQKPGQSPKLLIYWASTRHTGVPDRFTGSGSGTDYTLTISSVQAEDLALYYCQQHYSTPYTFGGGTKLEIK

SEQ ID NO: 805 - protein, 107 aa, synthetic construct
  REGION 1..107: note = VL-65
DIAMTQSHKFMSTSVGDRVSITCKASQDVSTAVAWYQQKPGQSPKLLIYWASTRHTGVPDRFTGSGSGTDYTLTISSVQAEDLALYYCQQHYNTPYTFGGGTKLEIK

SEQ ID NO: 806 - protein, 112 aa, synthetic construct
  REGION 1..112: note = VL-66
DVGMTQTPLILSVTIGQPASISCKSSQSLLYINGKTYLNWLLQRPGQSPKRLIYLVSKLDSGVPNRFSGSGSGTDFTLKISRVEAEDLGVYYCLQSTHFPQTFGGGTKLEIK

SEQ ID NO: 807 - protein, 107 aa, synthetic construct
  REGION 1..107: note = VL-67
DILLTQSPSSMSVSLGDTVSITCHASQGINSNIGWLQQKPGKSFKGLIYHGTNLEDGVPSRFSGSGSGADYSLTISSLESEDFADYYCVQYDQFPWTFGGGTKLEIK

SEQ ID NO: 808 - protein, 112 aa, synthetic construct; identical to SEQ ID NO: 806
  REGION 1..112: note = VL-68

SEQ ID NO: 809 - protein, 112 aa, synthetic construct
  REGION 1..112: note = VL-69
DIVMTQAAPSVPVTPGESVSISCRSSRSLLHSNGYTYLYWFLQRPGQSPRLLIYRMSNLASGVPDRFSGSGSGTAFTLRISRVEAEDVGVYYCMQHLEYPYTFGGGTKLEIK

SEQ ID NO: 810 - protein, 107 aa, synthetic construct
  REGION 1..107: note = VL-70
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPRTFGGGTKLEIN

SEQ ID NO: 811 - protein, 112 aa, synthetic construct
  REGION 1..112: note = VL-71
DVVMTQTPLTLSVTIGQPASISCKSSRSLLDSDGKTYLNWLLQRPGQSPKRLIYLVSKLDSGVPDRFTGSGSGTDFTLKISRVEAEDLGVYYCWQGTHFPQTFGGGTKLEIN

SEQ ID NO: 812 - DNA, 366 bp, synthetic construct
  misc_feature 1..366: note = 846.2D4 VH nucleic acid
  source 1..366: mol_type = other DNA
gaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaacttagattgaaatctaataattatgcaacacattatgcggagtctgtgaaagggaggttcaccatctcaagagatgattccaaaagtagtgtctacctgcaaatgaacaacttaagagctgaagacactggcatttattactgtaccaggcactacggctttccctactggtacttcgatgtctggggcgcagggaccacggtcaccgtctcctca

SEQ ID NO: 813 - DNA, 354 bp, synthetic construct
  misc_feature 1..354: note = 846.2F11 VH nucleic acid
  source 1..354: mol_type = other DNA
gaggtccagctgcagcagtctggacctgagctggtgaagcctggggcttcagtgaagatgtcctgcaaggcttctggatacacattcactagctatgttatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagattacttttttactatgctatggactactggggtcaaggaacctcagtcaccgtctcctca

SEQ ID NO: 814 - DNA, 354 bp, synthetic construct
  misc_feature 1..354: note = 846T.10B1 VH nucleic acid
  source 1..354: mol_type = other DNA
caggttcagctgcagcagtctggacctgagctggtgaggcctggggctttagtgaagatttcctgcaaggcttctggttacaccttcacaagctacgatataaactgggtgaagcagaggcctggacagggacttgagtggattggatggatttatcctggagatggtagtactaaatacaatgagaaattcaagggcaaggccacactgactgcagacaaatcctccagcacagtttacatgcagctcagcagcctgacttctgagaactctgcagtctatttctgtgctagagaggcggcttctaatgccatggactactggggtcaaggaacctcagtcaccgtctcctca

SEQ ID NO: 815 - DNA, 354 bp, synthetic construct
  misc_feature 1..354: note = 846T.2E3 VH nucleic acid
  source 1..354: mol_type = other DNA
caggtccagctgcagcagtctggacctgagctggtgaagcctggggctttagtgaagatatcctgcaaggcttctggttacaccttcacaagctacgatataaactgggtgaagcagaggcctggacagggacttgagtggattggatggatttatcctggagatggtagtactaagtacaatgagaaattcaagggcaaggccgcactgactgcagacaagtcctccagcacagcctacatgcagctcagcagcctgacttctgagaactctgcagtctatttctgtgcaagagaggcggcttctaatgctatggactactggggtcaaggaacctcagtcaccgtctcctca

SEQ ID NO: 816 - DNA, 354 bp, synthetic construct
  misc_feature 1..354: note = 846T.4C9 VH nucleic acid
  source 1..354: mol_type = other DNA
gaggtccagctgcagcagtctggacctgagctggtaaagcctggggcttcagtgaagatgtcctgcaaggcttctggatactcattcactaactatattatgcactgggtgaagcagaagcctgggcagggccttgagtggattggatatattaatccttacaatgatggtactaagtacaatgagaagttcaaaggcaaggccacactgacttcagacaaatcctccagcacagcctacatggagctcagcagcctgacctctgaggactctgcggtctattactgtgcaagattacttttttactatgctatggactactggggtcaaggaacctcagtcaccgtctcctca

SEQ ID NO: 817 - DNA, 354 bp, synthetic construct
  misc_feature 1..354: note = 846T.4E11 VH nucleic acid
  source 1..354: mol_type = other DNA
caggttcagctgcagcagtctggagctgaactggtaaagcctggggcttcagtgaagttgtcctgcaaggcttctggctacaccttcagaagctatgatataaactgggtgaggcagaggcctgaacagggacttgagtggattggatggatttttcctggagaaggtatttctaagtacaatgagaagttcaagggcaaggccacactgactacagacaaattgtccagcacagcctacatgcagctcagcagcctgacatctgaggactctgctgtctatttctgtgcaagagaggctacggctggggctatggactactggggtcaaggaacctcagtcaccgtctcctca

SEQ ID NO: 818 - DNA, 366 bp, synthetic construct
  misc_feature 1..366: note = 846T.4F5 VH nucleic acid
  source 1..366: mol_type = other DNA
gaggtgaagcttctcgagtctggaggaggcttggtgcaacctggaggatccatgaaactctcctgtgttgcctctggattcactttcagtaacttctggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccaggcactacggctttccctactggtacttcgatgtctggggcgcagggaccacggtcaccgtctcctca

SEQ ID NO: 819 - DNA, 354 bp, synthetic construct
  misc_feature 1..354: note = 846T.8D1 VH nucleic acid
  source 1..354: mol_type = other DNA
cagattcagttgcagcagtctggagctgaactggtaaagcctggggcttcagtgaagttgtcctgcaaggcttctggctacaccttcacaagctatgatatatgctgggtgaggcagaggcctgagcagggacttgagtggattggatggatttttcctggagatggtactactaagtacactgagaacttcaagggcaaggccacactgactacagacaaatcctccagcacagcctacatgcacctcagcaggctgacatctgaggactctgctgtctatttctgtgcaagggagggggcgggctcctggtttgcttactggggccaagggactctggtcactgtctctgca

SEQ ID NO: 820 - DNA, 342 bp, synthetic construct
  misc_feature 1..342: note = 847.10C9 VH nucleic acid
  source 1..342: mol_type = other DNA
gaggttcagctgcagcagtctggagctgagctggtgcggcctgggacttcagtgaaggtgtcctgcaaggcttcggaatacgccttcaataattacatgatggaatgggtaaaacagaggcctggacagggccttgagtggattggagtgattaatcccggaagtggtgctactaattacaatgagaatttcaaggacaaggcaacactgactgcagacaaatcctccaccactgcctacatgcagctcagcagcctgacatctgatgactctgcggtctatttctgtacctacaatgatcttggttactggggccaagggactctggtcactgtctctgca

SEQ ID NO: 821 - DNA, 369 bp, synthetic construct
  misc_feature 1..369: note = 847.11D6 VH nucleic acid
  source 1..369: mol_type = other DNA
gaggtcaagctgcagcagtctggacctgagctgatgaagcctggaacttcaatgaagatatcctgtaaggcttctggttactcattcaccggctacaccatgaactgggtgaagcagagccatggaaagaaccttgagtggattggacttattaatccttacaatggtggtactaactacaaccagaagttcaggggcaaggccacattaagtgtagacaagtcatccaacacagcctacatggagctcctcagtctgacatctgaagactctgcagtctattactgtgcaagaaaggacgattattacggaagtagtccctatgctctggactactggggtcaaggaacctcagtcaccgtctcctca

SEQ ID NO: 822 - DNA, 348 bp, synthetic construct
  misc_feature 1..348: note = 847.15D12 VH nucleic acid
  source 1..348: mol_type = other DNA
caggtccagctgcagcagtctggagctgagctgatgaagcctggggcctcagtgaagatatcctgcaaggctactggctacacattcactaactactggatagagtgggtaaagcagaggcctggacatggccttgaatggattggagaatttttacctggagatggcagtagtcactacaatgagaagttcaagggcaaggccacattcacttcagatacatcctccaacacagcctacatacacctcaccagcctgacatctgaggactctgccgtctattactgtgtaagagatgactacgacagtgactactgggcccaaggcaccactctcacagtctcctca

SEQ ID NO: 823 - DNA, 318 bp, synthetic construct
  misc_feature 1..318: note = 847.15F9 VH nucleic acid
  source 1..318: mol_type = other DNA
caaattgttctctcccagtctccagcaatcctgtctgcatctccaggggagaaggtcacaatgacttgcagggccagctcaagtgtaacttacatgtactggtaccagcagaagccaggatcctccccccaaccctggatttatgccacatccaacctggcttctggagtccctgctcgcttcagtggcagtgggtctgggacctcttactctctcacaatcagcagagtggaggctgaagatgctgccacttattactgccagcagtggagtagtaacccactcacgttcggtgctgggaccaagctggagctgaaa

SEQ ID NO: 824 - DNA, 366 bp, synthetic construct
  misc_feature 1..366: note = 847.15H11 VH nucleic acid
  source 1..366: mol_type = other DNA
gaggtccagcttcagcagtctggggctgaagttgtgaagcctggggctccagtgaagctgtcctgcaaggcttctggctacaccttcaacaactactggctgaactgggtgaaacagcggcctggacgaggcctcgagtggattggaaggattgatccttccgatagtgaaactcactacaatcaaaagttcaaggacaaggccacactgactgtagacatgtcctccagcacagcctacatccaactcagcagcctgacatctgaggactctgcggtctattactgtgcaagaggggattattattacggtactaggggtcctatggactactggggtcaaggcacctcagtcaccgtctcctca

SEQ ID NO: 825 - DNA, 345 bp, synthetic construct
  misc_feature 1..345: note = 847.20H7 VH nucleic acid
  source 1..345: mol_type = other DNA
caggtccagctgcagcagtctggggctgagctggtgaggcctggggcttcagtgaacctgtcctgcaaggcttctggctacaccttcaccaactactggatgaactgggtgaagcagaggcctggacaaggccttgaatgggttggtatgattgatccttcagacagtgaaactcactacaatcaaatgttcaaggacaaggccacattgactatagacaaatcttccagcatagcctacatgcagctcagcagcctgacatctgaggactctgcggtctattactgtgcaagatggggtgctatggactactggggtcaaggaacctcagtcaccgtctcctca

SEQ ID NO: 826 - DNA, 363 bp, synthetic construct
  misc_feature 1..363: note = 847.21B11 VH nucleic acid
  source 1..363: mol_type = other DNA
caggtgcaactgaagcagtcaggacctggcctggtggcgccctcacagagcctgtccatcacttgcactgtctctggattttcattaaccagctatggtgtacactgggttcgccagcctccaggaaagggtctggagtggctgggaataatatgggctggtggaagcacaaattataatccggctctcatgtccagactgagcatcagcaaagacaaatccaagagccaagttttcttaaaaatgaacagtctgaaaactgatgacacagccatgtactactgtgccagagatcgggcttcattactacggccgggggctatggactactggggtcaaggaacctcagtcaccgtctcctca

SEQ ID NO: 827 - DNA, 354 bp, synthetic construct
  misc_feature 1..354: note = 847.27B9 VH nucleic acid
  source 1..354: mol_type = other DNA
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacatatgctgaagagttcaagggacggtttgccttctctttggagacctctgccagcactgcctatttgcagatcaactacctcaaaaatgaggacacggctacatttttctgtgcaagattggactatgattacgacgaggactactggggccaaggcaccactctcacagtctcctca

SEQ ID NO: 828 - DNA, 360 bp, synthetic construct
  misc_feature 1..360: note = 847.28D1 VH nucleic acid
  source 1..360: mol_type = other DNA
caggtgcagcttgtagagaccggacctggcctggtggcgccctcacagagcctgtccatcacttgcactgtctctgggttttcattaaccagctatggtgtacactggattcgtcagtctccaggaaagggtctggagtggctgggaactatatgggctggtggaagcacagattataattcgccactcatgtccagactgagcatcagcaaagacaactccaagaaccaagtttttttaaaaatgaatagtctgcagattgatgacacagccatatattattgtgccagtcctccttctgggtatgattacgacgtgtttgcttcctggggccaagggactctggtcactgtctctgca

SEQ ID NO: 829 - DNA, 360 bp, synthetic construct
  misc_feature 1..360: note = 847.2B8 VH nucleic acid
  source 1..360: mol_type = other DNA
caggtacaactgaagcagtcaggacctggcctagtgcagccctcacagagcctgtccatcacctgcacagtctctggtttctcattaatcaactatggtgtacactgggttcgccaatctccaggaaagggtctggagtggctgggagtgatatggagtggtggaagcacagactataatgcagctttcatatccagactgagcatcagcaaggacaattccaagagccaagttttctttaaaatgaacagtctgcaagctgatgacacagccatatattactgtgccagaatttattactacggtaggggctatgctatggactactggggtcaaggaacctcagtcaccgtctcctca

SEQ ID NO: 830 - DNA, 360 bp, synthetic construct
  misc_feature 1..360: note = 847.3B3 VH nucleic acid
  source 1..360: mol_type = other DNA
caggtgcagctgaaggagtcaggacctggcctggtggcgccctcacagagcctgtccatcacttgcactgtctctgggttttcattaaccagctatggtgtacactggattcgccagcctccaggaaagggtctggagtggctgggaggaatatgggctggtggaagtacagattacaattcgcctctcatgtcccgactgagtatcactaaagacaactccaagagccaagttttcttaaaaatgaacagtctgcaaagtgatgacacagccatgtactactgtgccagtcctccctctgggtatgattacgacgtgtttgcttactggggccaagggactctggtcactgtctctgca

SEQ ID NO: 831 - DNA, 360 bp, synthetic construct
  misc_feature 1..360: note = 849.1D2 VH nucleic acid
  source 1..360: mol_type = other DNA
caggtgcagctgaaggagtcaggacctgtcctggtggcgccctcacagagcctgtccatcacttgcactgtctctgggttttcattaagcagctatggtgtacactgggttcgccagcctccaggaaagggtctggagtggctgggagtaatatgggctggtggaagcacagattatgattcagctctcatgtccagactgagcatcagcaaagacaactccaagagccaagttttcttaaaaatggacagtctgcaaactgatgacacagccatgtactactgtgccaaacccccatatgattacgacggggcctggtttgcttactggggccaagggactctggtcactgtctctgca

SEQ ID NO: 832 - DNA, 342 bp, synthetic construct
  misc_feature 1..342: note = 849.2D7 VH nucleic acid
  source 1..342: mol_type = other DNA
caggtgcaactgcagcagtctgggcctcagctggttaggcctggggcttcagtgaagatatcctgcaaggcttctggttactcattcaccaactactggatgcactgggtgaagcagaggcctggacaaggtcttgagtggattggcatgattgatccttccgatagtgaaactaggttaaatcagaagttcaaggacaaggccacattgactgtagacaaatcctccagcacagcctacatgcaactcagcagcccgacatctgaggactctgcggtctattactgtgcaacggggagacccgactactggggccaaggcaccactctcacagtctcctca

SEQ ID NO: 833 - DNA, 342 bp, synthetic construct
  misc_feature 1..342: note = 849.2F12 VH nucleic acid
  source 1..342: mol_type = other DNA
caggtgcaactgcagcagtctgggcctcagctggttaggcctggggcttcagtgaagatatcctgcaaggcttctggttactcattcaccagctactggatgcactgggtgaagcagaggcctggacaaggtcttgagtggattggcatgattgatccttccgatagtgaaactaggttaaatcagaagttcaaggacaaggccacattgactgtagacaaatcctccagcacagcctacatgcaactcagcagcccgacatctgaggactctatagtctattactgtgcaaaggggagacccgactactggggccaaggcaccactctcacagtctcctca

SEQ ID NO: 834 - DNA, 354 bp, synthetic construct
  misc_feature 1..354: note = 849.4B2 VH nucleic acid
  source 1..354: mol_type = other DNA
gatggaaaacttcaggagtcaggacctggcctcgtgaaaccttctcagtctctgtctctcacctgctctgtcactggctactccatcaccagtggttattactggaactggatccggcagtttccaggaaacaaactggaatggatgggccacataagctacgatggtagaaataactacaacccatctctcaaaaatcgaatctccatcactcgtgacacatctaagaaccagtttttcctgaagttgaattctgtgactactgaggacacagccacatattactgtgcaggctatgatttcctctggtacttcgatgtctggggcacagggaccacggtcaccgtctcctca

SEQ ID NO: 835 - DNA, 363 bp, synthetic construct
  misc_feature 1..363: note = 849.4F12 VH nucleic acid
  source 1..363: mol_type = other DNA
caggtgcagctgaaggagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataaacactataacccagccctgaagagccggctcacaatctccaaggatacctccaaaaaccaggtcttcctcaggatcgccaatgtggacactgcagataatgccacatattactgtgctcgaatcgactatggtgactacgtcgggtttgcttattggggccaagggactctggtcactgtctctgca

SEQ ID NO: 836 - DNA, 354 bp, synthetic construct
  misc_feature 1..354: note = 849.4F2 VH nucleic acid
  source 1..354: mol_type = other DNA
gatggacagcttcaggagtcaggacctggcctcgtgaaaccttctcagtctctgtctctcacctgctctgtcactggctactccatcaccagtggttattactggaactggatccggcagtttccaggaaacaaactggaatggatgggccacataagctacgatggtagaaataactacaacccatctctcaaaaatcgaatctccatcactcgtgacacatctaagaaccagtttttcctgaagttgaattctgtgactactgaggacacagccacatattactgtgcaggctatgatttcctctggtacttcgatgtctggggcacagggaccacggtcaccgtctcctca

SEQ ID NO: 837 - DNA, 357 bp, synthetic construct
  misc_feature 1..357: note = 849.5C2 VH nucleic acid
  source 1..357: mol_type = other DNA
gaggttcagctgcagcagtctggggcagagcttgtgaagtcaggggcctcagtcaagttgtcctgcacagcttctggcttcaacattaaagactactatatgcactgggtgaagcagaggactgaacagggcctggagtggattggaaggatttatcctgaggatgatgaaactaaatatgccccgaaattccagggcaaggccactataacagcagacacatcctccaacacagcctacctgcagctcagcagcctgacatctgaggacactgccgtctattactgtgctagatcagggggttactacgtttggtttgcttactggggccaagggactctggtcactgtctctgca

SEQ ID NO: 838 - DNA, 363 bp, synthetic construct
  misc_feature 1..363: note = 849.8D12 VH nucleic acid
  source 1..363: mol_type = other DNA
gaggtccagctacaacagtctggacctgagctggtgaagcctgggtcttcagtgaagatatcctgcaaggcttctagatacacattcactgactacaacatggactgggtgaagcagagccatggaaagagacttgagtggattggatatatttatcctaacaatggtggtactggctacaaccagaagttcaagggcaaggccacattgactgtagacaagtcctccagcacagcctacatggagctccgcagcctgacatctgaggactctgcagtctattactgtgcaaggtcctactacgatggtagctacgatgctatggactactggggtcaaggaacctcagtcaccgtctcctca

SEQ ID NO: 839 - DNA, 342 bp, synthetic construct
  misc_feature 1..342: note = F847C.21H6 VH nucleic acid
  source 1..342: mol_type = other DNA
cagatccagttggtgcagtctggacctgagctgaagaagcctggagagacagtcaagatctcctgcaaggcttctgggtataccttcacaatctttggaatgaactgggtgaagcaggctccaggaaagggtttaaagtggatgggctgggtaaacacctacactggagagccaacatatgctgatgacttcaagggacggtttgccttctctttggaaacctctgccagcactgcctatttgcagatcaacaacctcaaaaatgaggacacggctacatatttctgtgcaagaggctggtttgcttactggggccaagggactctggtcactgtctctgca

SEQ ID NO: 840 - DNA, 333 bp, synthetic construct
  misc_feature 1..333: note = 846.2D4 VL nucleic acid
  source 1..333: mol_type = other DNA
aacattgtgctcacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaataaaa

SEQ ID NO: 841 - DNA, 321 bp, synthetic construct
  misc_feature 1..321: note = 846.2F11 VL nucleic acid
  source 1..321: mol_type = other DNA
gacatcctgatgacccaatctccatcctccatgtctgtatctctgggagacacagtcagcatcacttgccatgcaagtcagggcattaacagtaatatagggtggttgcagcagaaaccagggaagtcatttaagggcctgatctatcatggaaccaacttggaagatggagttccatcaaggttcagtggcagtggatctggagcagattattctctcaccatcagcagcctggaatctgaagattttgcggactattactgtgtacagtatgatcagtttccgtggacgttcggtggaggcaccaagctggaaatcaaa

SEQ ID NO: 842 - DNA, 333 bp, synthetic construct
  misc_feature 1..333: note = 846T.10B1 VL nucleic acid
  source 1..333: mol_type = other DNA
gacattgtgatgtcacagtctcctgcttccttaggtgtttctctggggcagagggccaccatctcatgcagggccagcaaaagtgtcagtacatctggctatagttatatgcactggtaccaacagaaaccaggacagccacccaaactcctcatctatcttgcatccatcctagaatctggggtccctgccaggttcagtggcagtgggtctgggacagacttcaccctcaacatccatcctgtggaggaggaggatgctgcaatctattactgtcagcacagtagggagcttcctctcacgttcggtgctgggaccaagctggagctgaaa

SEQ ID NO: 843 - DNA, 333 bp, synthetic construct
  misc_feature 1..333: note = 846T.2E3 VL nucleic acid
  source 1..333: mol_type = other DNA
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccatcctagaatctggggtccctgccaggttcagtggcagtgggtctgggacagacttcaccctcaacatccatcctgtggaggaggaggatgctgcaacctattactgtcagcacagtagggagcttcctctcacgttcggtgctgggaccaagctggagctgaaa

SEQ ID NO: 844 - DNA, 321 bp, synthetic construct
  misc_feature 1..321: note = 846T.4C9 VL nucleic acid
  source 1..321: mol_type = other DNA
gacattttgatgacccaaactccatcctccatgtctgtatctctgggagacacagtcagcatcacttgccatgcaagtcagggcattagcagtaatatagggtggttgcagcagaaaccacggaaatcatttaagggcctgatctattatggaaccaacttggaagatggagttccatcaaggttcagtggcagtggatctggagcagattattctctcaccatcagcagcctggaatctgaagattttgcagactattactgtgtacagtatgatcagtttccgtggacgttcggtggaggcaccaagctggaaataaaa

SEQ ID NO: 845 - DNA, 333 bp, synthetic construct
  misc_feature 1..333: note = 846T.4E11 VL nucleic acid
  source 1..333: mol_type = other DNA
gacattgtgctgacacagtctcctgcttccttagctgtatctctggggcagagggccaccatctcatgcagggccagcaaaagtgtcagtacatctggctatagttgtatgcactggtaccaacagaaaccaggacagccacccaaactcctcatctatcttgcatccaacctagaatctggggtccctgccaggttcagtggcggtgggtctgggacagacttcaccctcagcatccatcctgtggaggaggaggatgctgcaacctattactgtcaccacagtacggagcttccgtacacgttcggaggggggaccaagctggaagtagaa

SEQ ID NO: 846 - DNA, 333 bp, synthetic construct
  misc_feature 1..333: note = 846T.4F5 VL nucleic acid
  source 1..333: mol_type = other DNA
gacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaa

SEQ ID NO: 847 - DNA, 324 bp, synthetic construct
  misc_feature 1..324: note = 846T.8D1 VL nucleic acid
  source 1..324: mol_type = other DNA
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccatcctagaatctggggtccctgccaggttcagtggcagtgggtctgggacagacttcaccctcaacatccatcctgtggaggaggaggatgctgcaacctattactgtcagcacagtagggagcttcctctcacgttcggtgctgggaccaagctg

SEQ ID NO: 848 - DNA, 333 bp, synthetic construct
  misc_feature 1..333: note = 847.10C9 VL nucleic acid
  source 1..333: mol_type = other DNA
gatgttgtgatgacccagactccactcactttgtcggttaccattggacaaccagcctctatctcttgcaagtcaagtcagagcctcttatatagtgatggaaaaacctatttgaattggttattacagaggccaggccagtctccaaagcgcctaatctatctggtgtctcaactggactctggagtccctgacaggttcactggcagtggatcaggaacagattttacactgaaaatcagcagagtggaggctgatgatttgggagtttattgctgcgtgcaaggtacacatcttcccacgttcggaggggggaccaagctggaaataaaa

SEQ ID NO: 849 - DNA, 333 bp, synthetic construct
  misc_feature 1..333: note = 847.11D6 VL nucleic acid
  source 1..333: mol_type = other DNA
gacattgtgatgacacagtctcccgcttctttggctgtgtctctagggcagagggccaccatatcctgcagagccagtgaaagtgttgaaaatcatgtcaatagttttatgcactggttccaacagaaaccaggacagccacccaaactcctcatctatcgtgcatccaacctacaatctgggatccctgccaggttcagtggcagtgggtctaggacagacttcaccctcaccattaatcctgtggaggctgatgatgttgcaacctattactgtcagcaaaataatgaggtcccgtacacgttcggaggggggaccaagctggaaataaaa

SEQ ID NO: 850 - DNA, 327 bp, synthetic construct
  misc_feature 1..327: note = 847.15D12 VL nucleic acid
  source 1..327: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccaaactgactgtccta

SEQ ID NO: 851 - DNA, 360 bp, synthetic construct
  misc_feature 1..360: note = 847.15F9 VL nucleic acid
  source 1..360: mol_type = other DNA
tcccagtctccagcaatcctgtctgcatctccaggggagaaggtcacaatgacttgcagggccagctcaagtgtaacttacatgtactggtaccagcagaagccaggatcctccccccaaccctggatttatgccacatccaacctggcttctggagtccctgctcgcttcagtggcagtgggtctgggacctcttactctctcacaatcagcagagtggaggctgaagatgctgccacttattactgccagcagtggagtagtaacccactcacgttcggtgctgccagaatttattactacggtaggggctatgctatggactacgtgttcatcttccctcccagcgacgagcagctg

SEQ ID NO: 852 - DNA, 333 bp, synthetic construct
  misc_feature 1..333: note = 847.15H11 VL nucleic acid
  source 1..333: mol_type = other DNA
gacattgtgctaacacagtctccagcttctttggctgtgtctctagggcagagggccaccatctcctgcagagccagcgaaagtcttgataattctggcattagttttatgaactggttccaacagagaccaggacagccacccaaactcctcatctatgctgcatccaacctagaatccggggtccctgccaggtttagtggcagtgggtctgggacagacttcagcctcaacatccatcctatggaggaggatgatgctgcaatgtatttctgtcagcacagtaaggaggttccgtggacgttcggtggaggcaccaagctggaaatcaaa

SEQ ID NO: 853 - DNA, 321 bp, synthetic construct
  misc_feature 1..321: note = 847.20H7 VL nucleic acid
  source 1..321: mol_type = other DNA
gacattgtgatgacccagtctcacaaaatcatgtccacatcagtaggagacagggtcagcatcacctgcaaggccagtcaggatgtgagtaccactgtggcctggtttcagcaaaaaccaggacaatctcctaaactactgctttactgggcatccacccggcacactggagtccctgatcgcttcacaggcagtggatctgggacagattatactctcaccatcagcagtgtccaggctgaagacctggcactttattactgtcagcaacattatagccctccattcacgttcggctcggggaccaagctggaaataaaa

SEQ ID NO: 854 - DNA, 321 bp, synthetic construct
  misc_feature 1..321: note = 847.21B11 VL nucleic acid
  source 1..321: mol_type = other DNA
gacattgtgatgactcagtctcaaaaattcatgtccacatcagtaggagacagggtcagcgtcacctgcaaggccagtcagaatgtgggtattaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataatagttatccgtacacgttcggaggggggaccaagctggaaatgaaa

SEQ ID NO: 855 - DNA, 336 bp, synthetic construct
  misc_feature 1..336: note = 847.27B9 VL nucleic acid
  source 1..336: mol_type = other DNA
gacgttgtgatgacccagactccactcactttgtcggttacccttggacaacccgcctccatctcttgcaggtcaagtcagagcctcttagatagtgatggaaagacatatttgaattggttgttacagaggccaggccagtctccaaagcgcctaatctatttggtgtctagactggactctggagtccctgacaggttcactggcagtggatcagggacagatttcacactgaaaatcagcagagtggaggctgaggatctgggagtttattattgctggcaaggtacacattttccgttcacgttcggaggggggaccaagctggaaataaaa

SEQ ID NO: 856 - DNA, 321 bp, synthetic construct
  misc_feature 1..321: note = 847.28D1 VL nucleic acid
  source 1..321: mol_type = other DNA
gacatccggatgactcagtctccagcctccctatctgcatctgtgggagaaactgtcaccatcacatgtcgagcaagtgggaatattcacaattatttagcatggtatcatcagaaacagggaaaatctcctcagctcctggtctataatgtaaaaaccttagcagatggtgtgccatcaaggttcagtggcagtggatcaggaacacaattttctctcaagatcaacagtttgcagcctgaagattttgggacttattattgtcaacatttttggagtatttttccgacgttcggtggaggcaccaagctggaaataaaa

SEQ ID NO: 857 - DNA, 318 bp, synthetic construct
  misc_feature 1..318: note = 847.2B8 VL nucleic acid
  source 1..318: mol_type = other DNA
caaattcttctctcccagtctccagcaatcctgtctgcatctccaggggagaaggtcacaatgacttgcagggccagctcaagtgtaacttacatgtactggtaccagcagaagccaggatcctccccccaaccctggatttatgccacatccaacctggcttctggagtccctgctcgcttcagtggcagtgggtctgggacctcttactctctcacaatcagcagagtggaggctgaagatgctgccacttattactgccagcagtggagtagtaacccactcacgttcggtgctgggaccaagctggagctgaaa

SEQ ID NO: 858 - DNA, 315 bp, synthetic construct
  misc_feature 1..315: note = 847.3B3 VL nucleic acid
  source 1..315: mol_type = other DNA
gacattttgatgactcagtctccagcctccctatctgcatctgtgggagaaactgtcaccatcacatgtcgagcaagtgggaatattcacaattatttagcatggtatcagcagaaacagggaaaatctcctcaactcctggtctatagtgcaaaaaccttagcagatggtgtgccatcaaggttcagtggcagtggatcaggaacacaattttctctcaagatcgacagcctgcagcctgaagattttgggacttattactgtcaacatttttggagtctttttccgacgttcggtggaggcaccaagctggaa

SEQ ID NO: 859 - DNA, 321 bp, synthetic construct
  misc_feature 1..321: note = 849.1D2 VL nucleic acid
  source 1..321: mol_type = other DNA
gacgttgtgatgacccagactcccaaattcctgcttgtatcagcaggagacagggttaccataacctgcaaggccagtcaaagtgtgagtagtgatgtagcttggtaccaacagaagccagggcagtctcctaaactgctgatatactttgcatccaatcgctacactggagtccctgatcgcttcactggcagtggatatgggacggatttcactttcaccatcagcactgtccaggctgaagacctggcagtttatttctgtcagcataattatatctctccgttcacgttcggaggggggaccaagctggaaataaaa

SEQ ID NO: 860 - DNA, 321 bp, synthetic construct
  misc_feature 1..321: note = 849.2D7 VL nucleic acid
  source 1..321: mol_type = other DNA
gacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattatagcactccgtacacgttcggaggggggaccaagctggaaataaaa

SEQ ID NO: 861 - DNA, 321 bp, synthetic construct
  misc_feature 1..321: note = 849.2F12 VL nucleic acid
  source 1..321: mol_type = other DNA
gacattgcgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattataacactccgtacacgttcggaggggggaccaagctggaaataaaa

SEQ ID NO: 862 - DNA, 336 bp, synthetic construct
  misc_feature 1..336: note = 849.4B2 VL nucleic acid
  source 1..336: mol_type = other DNA
gatgtggggatgacccagactccactcattttgtcggttaccattggacaaccagcttccatctcttgcaagtcaagtcagagcctcttatatattaatggaaaaacctatttgaattggttattacagaggccaggccagtctccaaaacgcctaatctatctggtgtctaaattggactctggagtccctaacagattcagtggcagtggatcagggacagatttcacactgaaaatcagcagagtggaggctgaggatttgggagtttattactgcttgcagagtacacattttcctcagacgttcggtggaggcaccaagctggaaatcaaa

SEQ ID NO: 863 - DNA, 321 bp, synthetic construct
  misc_feature 1..321: note = 849.4F12 VL nucleic acid
  source 1..321: mol_type = other DNA
gacatcctgctgacccagtctccatcctccatgtctgtatctctgggagacacagtcagcatcacttgccatgcaagtcagggcattaacagtaatatagggtggttgcagcagaaaccagggaagtcatttaagggcctgatctatcatggaaccaacttggaagatggagttccatcaaggttcagtggcagtggatctggagcagattattctctcaccatcagcagcctggaatctgaagattttgcggactattactgtgtacagtatgatcagtttccgtggacgttcggtggaggcaccaagctggaaatcaaa

SEQ ID NO: 864 - DNA, 336 bp, synthetic construct
  misc_feature 1..336: note = 849.4F2 VL nucleic acid
  source 1..336: mol_type = other DNA
gatgttgggatgacccagactccactcattttgtcggttaccattggacaaccagcttccatctcttgcaagtcaagtcagagcctcttatatattaatggaaaaacctatttgaattggttattacagaggccaggccagtctccaaaacgcctaatctatctggtgtctaaattggactctggagtccctaacagattcagtggcagtggatcagggacagatttcacactgaaaatcagcagagtggaggctgaggatttgggagtttattactgcttgcagagtacacattttcctcagacgttcggtggaggcaccaagctggaaatcaaa

SEQ ID NO: 865 - DNA, 336 bp, synthetic construct
  misc_feature 1..336: note = 849.5C2 VL nucleic acid
  source 1..336: mol_type = other DNA
gatattgtgatgactcaggctgcaccctctgtacctgtcactcctggagagtcagtatccatctcctgcaggtctagtaggagtctcctgcatagtaatggctacacttacttgtattggttcctgcagaggccaggccagtctcctcggctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaagctggaaataaaa

SEQ ID NO: 866 - DNA, 318 bp, synthetic construct
  misc_feature 1..318: note = 849.8D12 VL nucleic acid
  source 1..318: mol_type = other DNA
gacatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttcctcggacgttcggtggaggcaccaagctggaaatc

SEQ ID NO: 867 - DNA, 336 bp, synthetic construct
  misc_feature 1..336: note = F847C.21H6 VL nucleic acid
  source 1..336: mol_type = other DNA
gatgttgtgatgacccagactccactcactttgtcggttaccattggacaaccagcctccatctcttgcaagtcaagtcggagcctcttagatagtgatggaaagacatatttgaattggttgttacagaggccaggccagtctccaaagcgcctaatctatctggtgtccaaactggactctggagtccctgacaggttcactggcagtggatcagggacagatttcacactgaaaatcagcagagtggaggctgaggatttgggagtttattattgctggcaaggtacacattttcctcagacgttcggtggaggcaccaagctggaaatcaat